(19) United States
(12) Patent Application Publication
(10) Pub. No.: US 2026/0217822 A1
(43) Pub. Date: Jul. 30, 2026

(54) CD19-SPECIFIC ANTIBODY CONSTRUCTS AND COMPOSITIONS THEREOF

(71) Applicant: Sana Biotechnology, Inc., Seattle, WA (US)

(72) Inventors: Jeremy KINDER, Seattle, WA (US); Neal VAN HOEVEN, Seattle, WA (US); Hallee A. WRIGHT, Seattle, WA (US); Jesse GREEN, Seattle, WA (US); Adam J. JOHNSON, Seattle, WA (US)

(21) Appl. No.: 19/146,350

(22) PCT Filed: Jan. 10, 2024
(86) PCT No.: PCT/US2024/011053

§ 371 Date: Jul. 8, 2025

Related U.S. Application Data
(60) Provisional application No. 63/479,328, filed on Jan. 10, 2023.

(51) Int. Cl. C07K 16/28; A61K 35/17; A61K 40/31; A61K 40/42; A61K 40/50; A61P 35/00; C07K 14/705; C07K 14/725; C12N 5/0783; C12N 7/00; C12N 9/22; C12N 15/11; C12N 15/86

(52) U.S. Cl. CPC C07K 16/2803; A61K 35/17; A61K 40/31; A61K 40/4211; A61K 40/50; A61P 35/00; C07K 14/7051; C07K 14/70517; C07K 14/70521; C07K 14/70596; C12N 5/0636; C12N 7/00; C12N 9/226; C12N 15/111; C12N 15/86; A61K 2239/13; A61K 2239/17; A61K 2239/21; A61K 2239/22; A61K 2239/25; A61K 2239/29; C07K 2317/24; C07K 2317/31; C07K 2317/565; C07K 2317/622; C07K 2317/92; C12N 2510/00; C12N 2760/18243; C12N 2830/20

(57) ABSTRACT

Disclosed herein are antibodies or antigen binding fragments thereof that specifically bind human CD19. Also disclosed are chimeric antigen receptors and chimeric antigen receptor transgenes comprising an antigen binding domain that specifically binds human CD19. Also described herein are immune cells, viral vectors, and other compositions containing the antibodies, antibody binding fragments, chimeric antigen receptors, and/or chimeric antigen receptor transgenes.

### RELATED APPLICATIONS

This application claims priority under 35 U.S.C. § 119(e) to U.S. Provisional Application No. 63/479,328, filed Jan. 10, 2023. The contents of this application are incorporated herein by reference in its entirety.

### SEQUENCE LISTING

The instant application contains a Sequence Listing which has been submitted electronically in ASCII format and is hereby incorporated by reference in its entirety. Said ASCII copy, created on Jan. 10, 2024, is named 15147_6006-00000_SL.xml and is 248 kilobytes in size.

### FIELD

The present disclosure relates to antibodies or antigen binding fragments thereof that specifically bind human CD19. Also disclosed are chimeric antigen receptors and chimeric antigen receptor transgenes comprising an antigen binding domain that specifically binds human CD19. Also disclosed are immune cells, viral vectors, and other compositions containing the antibodies, antibody binding fragments, chimeric antigen receptors and/or chimeric antigen receptor transgenes. Also disclosed are fusion proteins comprising a Henipavirus glycoprotein G and a human CD19 antibody, or an antigen binding fragment thereof. Viral vectors and other compositions containing the antibodies or antigen binding fragments thereof, chimeric antigen receptors and chimeric antigen receptor transgenes, and fusion proteins are disclosed. The present disclosure additionally relates to cells expressing chimeric antigen receptors, as well as methods of delivering the various antibodies and chimeric antigen receptors and methods of using cells expressing the chimeric antigen receptors.

### INTRODUCTION

Cluster of Differentiation 19 (CD19), also known as B-lymphocyte antigen CD19, is a transmembrane protein in the immunoglobin (1g) superfamily expressed on cells of the B cell lineage. CD19 is expressed during all phases of B cell development until terminal differentiation into plasma cells. Notably, expression of CD19 is regulated, with mature B cells expressing more CD19 than immature B cells. The expression of CD19 has been used as a marker in the diagnosis of a number of cancers, such as B cell lymphomas, acute lymphoblastic leukemia (ALL), and chronic lymphocytic leukemia (CLL). T lymphocytes are among the prime targets in gene therapy, even more so since chimeric antigen receptor (CAR) T cells have reached the clinic. Genetically modifying T cells with CAR constructs is the most common approach to creating tumor-specific T cells. The use of modified T cells is an emerging cell therapy approach within the area of adoptive cell transfer (ACT). This approach involves collecting T cells from a patient (autologous) or healthy donors (allogeneic), genetically modifying or engineering these T cells, and transferring the modified or engineered T cells into the patient to treat a range of diseases. The use of allogeneic T cells has several advantages over the use of autologous T cells, as the latter suffers from challenges such as a patient having insufficient healthy T cells for harvesting and the patient experiencing disease progression, co-morbidities, or even death in the time it takes to manufacture the engineered T cells. Additionally, CAR-T cells engineered with only human components can limit host immunogenicity that is induced by xenogenic CARs. Methods that efficiently produce effective CAR-T cels, including allogeneic CAR-T cells, targeting specific tumor antigens are needed. The present disclosure addresses this need.

There is a significant unmet need for novel CAR-T cells designed to treat B cell malignancies, including multiple myeloma. Of those receiving CAR-T therapies, many do not respond to treatment or relapse. Further, many receiving CAR-T therapies develop humoral immunity against available CAR-T therapeutics. For many patients, current manufacturing methods and capabilities present significant challenges for availability and access of CAR-T therapeutics, including those targeting B-cell malignancies. Thus, novel CAR-T cells for the treatment of patients with B-cell malignancies, like multiple myeloma, through the targeting of human CD19 and other potential secondary antigens are needed.

### BRIEF SUMMARY

The present disclosure provides an isolated polypeptide that specifically binds human cluster of differentiation 19 (CD19). In some embodiments, the isolated polypeptide comprises certain heavy chain variable regions (VH) and/or certain light chain variable regions (VL). In some embodiments, the isolated polypeptide comprises certain heavy chain complementarity determining regions (HCDR1, HCDR2, and HCDR3) and/or certain light chain complementarity determining regions (LCDR1, LCDR2, and LCDR3).

The present disclosure provides an antibody or antigen binding fragment thereof that specifically binds human Cluster of Differentiation 19 (CD19). In some embodiments, the antibody or antigen binding fragment thereof comprises certain heavy chain variable regions (VH) and/or certain light chain variable regions (VL). In some embodiments, the antibody or antigen binding fragment thereof comprises certain heavy chain complementarity determining regions (HCDR1, HCDR2, and HCDR3) and/or certain light chain complementarity determining regions (LCDR1, LCDR2, and LCDR3). The disclosure likewise provides for isolated polynucleotides, vectors, and host cells comprising the anti-CD19 antibody or antigen binding fragment thereof.

The present disclosure also provides a chimeric antigen receptor (CAR) that specifically binds human Cluster of Differentiation 19 (CD19). In some embodiments, the CAR comprises at least one of a signal peptide, an extracellular binding domain, a hinge domain, a transmembrane domain, an intracellular costimulatory domain, and/or an intracellular signaling domain. In some embodiments, the CAR extracellular binding domain comprises an antigen binding domain that comprises the antibody or antigen binding fragment thereof disclosed herein. The disclosure likewise provides for isolated polynucleotides, vectors, and host cells comprising the human anti-CD19 CAR.

The present disclosure also provides a viral vector targeting an immune cell, wherein the vector comprises an antibody or antigen binding fragment thereof that binds to a cell surface molecule on the immune cell and at least one polynucleotide encoding a chimeric antigen receptor (CAR) as disclosed herein. In some embodiments, the antibody or antigen binding fragment thereof binds to CD4 or CD8. In some embodiments, the vector comprises a henipavirus F protein molecule or a biologically active portion thereof. In some embodiments, the vector comprises a henipavirus envelope glycoprotein G (G protein) or a biologically active portion thereof. In some embodiments, the antibody or antigen binding fragment thereof that binds to a cell surface molecule is attached to a membrane-bound protein in the viral vector envelope. In some embodiments, the antibody or antigen binding fragment thereof that binds to a cell surface molecule is attached to a fusogen on the outer surface of the viral vector.

The present disclosure also provides a fusion protein comprising a henipavirus envelope glycoprotein G (G protein) or a biologically active portion thereof and an anti-CD19 antibody or antigen binding fragment thereof as herein disclosed.

The present disclosure provides a method for selectively modulating the activity of an immune cell, comprising delivery to an immune cell an effective amount of a viral vector comprising a polynucleotide encoding a CAR as disclosed herein. The present disclosure also provides a method for producing a chimeric antigen receptor (CAR) immune cell, comprising delivery to an immune cell an effective amount of a viral vector comprising a polynucleotide encoding a CAR as disclosed herein. In some embodiments, the immune cell is a T cell. In some embodiments the T cell is a primary T cell. In some embodiments, the polynucleotide encoding a CAR as disclosed herein is inserted into a site-specific locus. In some embodiments, the polynucleotide encoding a CAR as disclosed herein is inserted by homology-directed repair. In some embodiments, the immune cell expresses one or more CARs as disclosed herein.

The present disclosure additionally provides an engineered cell, comprising a CAR as herein disclosed and one or more modifications that (i) reduce expression of one or more MHC class I molecules and/or one or more MHC class II molecules, and/or (ii) increase expression of one of more tolerogenic factors, wherein the reduced expression of (i) and the increase expression of (ii) is relative to a cell of the same cell type that does not comprise the modifications.

The present disclosure additionally provides a method of administering to a subject in need thereof an effective amount of the CAR cells disclosed herein. The present disclosure also provides a method for treating a disease in a subject. The present disclosure provides a population of immune cells expressing the CARs disclosed herein. The present disclosure provides a composition of immune cells expressing the CARs disclosed herein. The present disclosure also provides a pharmaceutical composition of immune cells expressing the CARs disclosed herein. The present disclosure provides the use of the cells or the method disclosed herein for the treatment of a disease. In some embodiments, the disease is cancer. In some embodiments, the cancer is a hematologic malignancy. In some embodiments, the cancer is a solid malignancy.

### BRIEF DESCRIPTION OF DRAWINGS

FIGS. 1A-1B depict the in vitro characterization of Cluster of Differentiation 19 (CD19) chimeric antigen receptor constructs in human T cells.

FIGS. 2A-2E depict the effect of Cluster of Differentiation 19 (CD19) chimeric antigen receptor constructs on NALM-6 tumor cells at varying effector:target cell ratios.

FIG. 3A shows the binding of CD19 Binder 1 to recombinant CD19 in an enzyme-linked immunosorbent assay (ELISA). FIG. 3B shows the binding of CD19 Binder 2 to recombinant CD19 in an ELISA. FIG. 3C shows the binding of CD19 Binder 3 to recombinant CD19 in an ELISA.

FIGS. 4A-4C show binding of CD19 Binder 1 to two different cell types. FIG. 4A shows CD19 Binder 1 binding to CD19′ Raji cells. FIG. 4B shows CD19 Binder 1 binding to CD19⁻ 293 cells. FIG. 4C depicts the EC₅₀ for CD19 Binder 1.

FIGS. 5A-5C show binding of CD19 Binder 2 to two different cell types. FIG. 5A shows CD19 Binder 2 binding to CD19* Raji cells. FIG. 5B shows CD19 Binder 2 binding to C19⁻293 cells. FIG. 6C depicts the EC₅₀ for CD19 Binder 2.

FIGS. 6A-6C show binding of CD19 Binder 3 to two different cell types. FIG. 6A shows CD19 Binder 3 binding to CD19′ Raji cells. FIG. 6B shows CD19 Binder 3 binding to CD19⁻293 cells. FIG. 6C depicts the EC₅₀ for CD19 Binder 3.

FIG. 7A depicts in vivo tumor growth as measured by flux. FIG. 7B shows total area under curve for survival of mice receiving CD19 CAR-T cells after tumor introduction. FIG. 7C shows survival of mice receiving CD19 CAR-T cells after tumor introduction.

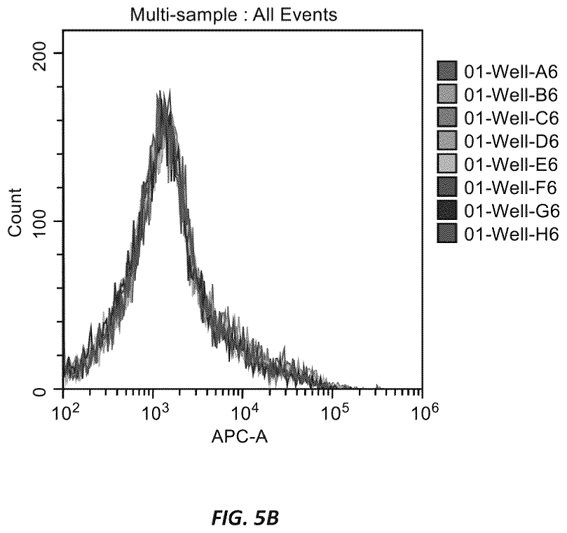

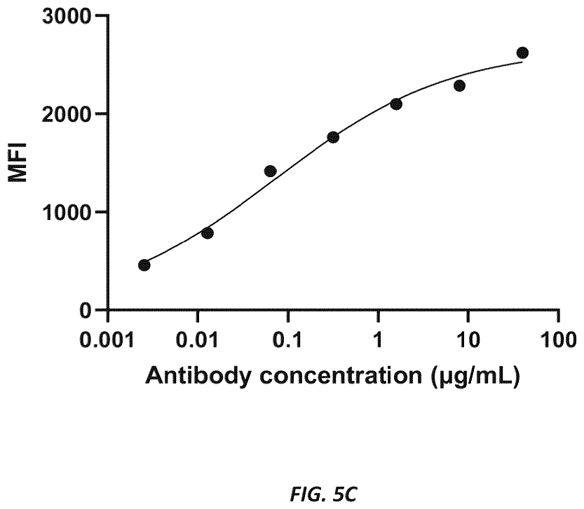

FIG. 5A illustrates the effect of administration of a CD8 targeted fusosome comprising the fully humanized CAR 400 (VL-VH) on tumor growth in vivo. FIG. 8B shows tumor radiance in mice receiving mock administration or the fully humanized CAR 400 (VL-VH).

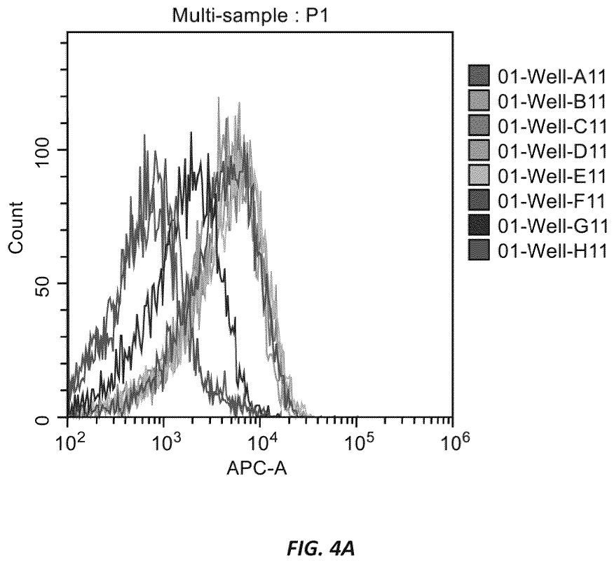

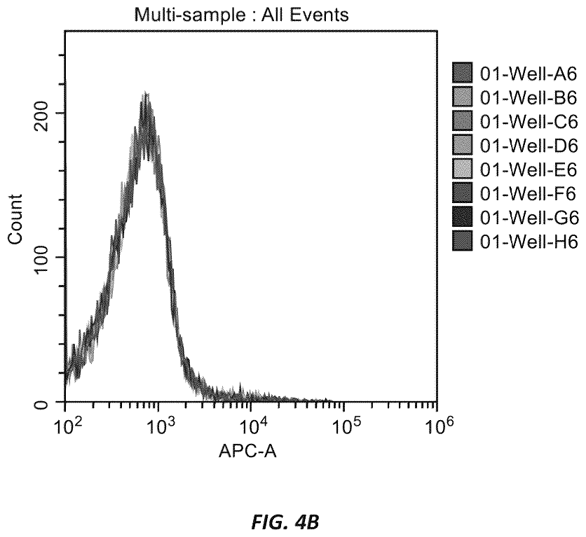

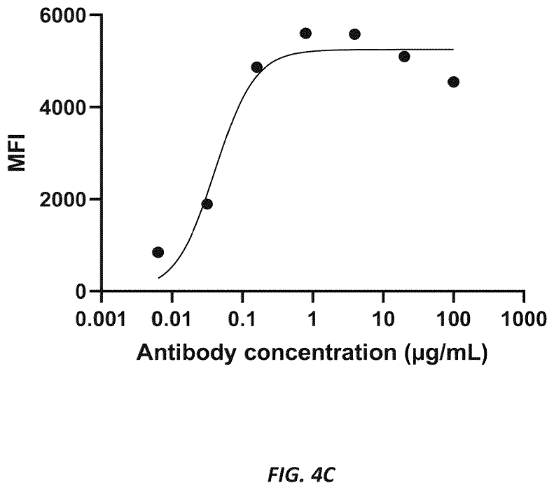

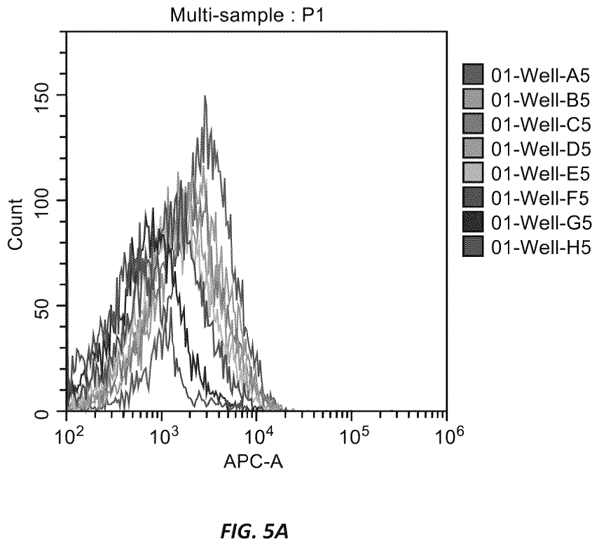

FIGS. 9A-4F show the transduction rate of activated PBMCs transduced with CD8-retargeted fusogens comprising a FMC63, CAR400 VLVH, or CAR400 VHVL CAR across different PBMC donors.

FIGS. 10A-10C show the vector copy number (VCN) per target cell genome in activated PBMCs transduced with CD8-retargeted fusogens comprising a FMC63, CAR400 VLVH, or CAR400 VHVL CAR across different PBMC donors.

FIG. 11 shows representative flow cytometry plots depicting CAR expression (FMC63, CAR400 VLVH, and CAR400 VHVL) in transduced activated PBMCs.

FIGS. 12A-12F show CAR-mediated killing of NAML6 cells for three different CD8-retargeted fusogens having different CD19 CARS (FMC63, CAR400 VLVH, and CAR400 VHVL). FIGS. 12D-12F depict the results of FIGS. 12A-12C normalized to CAR+ cells.

FIGS. 13A-13F show the transduction rate of resting PBMCs (e.g., an extracorporeal dosing setting) that were transduced with CD8-retargeted fusogens comprising a FMC63, CAR400 VLVH, or CAR400 VHVL CAR across different PBMC donors (FIGS. 13A-13C). FIGS. 13D-13F show the vector copy number (VCN) per target cell genome for resting PBMCs (e.g., an extracorporeal dosing setting) that were transduced with CD8-retargeted fusogens comprising a FMC63, CAR400 VLVH, or CAR400 VHVL CAR across different PBMC donors.

FIG. 14 shows an additional assessment of the transduction rate of resting PBMCs (e.g., an extracorporeal dosing setting) that were transduced with CD8-retargeted fusogens comprising a FMC63 control #1, CAR400 VLVH, CAR400 VHVL CAR, or FMC63 control #2.

FIGS. 15A-15L show total flux results in the B-cell tumor animal model, where animals received PBMCs from donor 1603C, and also received a range of doses of CAR400 VLVH, CAR400 VHVL, and FMC63 after administration of Nalm6:Wasabi-ffLuc cells.

FIGS. 16A-16L show total flux results in the B-cell tumor animal model, where animals received PBMCs from donor 3001C, and also received a range of doses of CAR400 VLVH, CAR400 VHVL, and FMC63 after administration of Nalm6:Wasabi-ffLuc cells.

FIGS. 17A and 176 show the area under the curve (AUC) for the tumor burden in animals receiving the different CARs (CAR400 VLVH, CAR400 VHVL, and FMC63) through 34 study days.

FIGS. 16A-18E shows total number and percentage CAR positive cells within peripheral blood CD4+ or CD8+ cell populations. FIGS. 16A and 188 show relative counts of CD4+ and CD8+ cells, respectively, in animals at study day 14, and FIGS. 18C and 18D show the percentage of CAR positive cells in total CD4+ and CD8+ cells, respectively. FIG. 18E shows the percentage of tumor cells detected in the peripheral blood of animals.

FIGS. 19A-19C show plasmid maps corresponding to CD47-FMC63, CD47-CAR400 VHVL, and CD47-CAR400 VLVH CARs, respectively.

FIGS. 20A-20C show confirmation of CAR transduction in target cells. FIG. 20A shows a representative gating strategy for flow cytometry of the CD47-CD19 hypoimmune CAR T cells. FIG. 20B shows flow cytometry based (QIFI) quantification of surface CD47 protein expression on CAR positive T cells, and FIG. 20C shows integration of the CAR construct into the target cell calculated by digital-droplet PCR.

FIGS. 21A-21C show physical and functional titers of the produced CD47-CD19 VSV-g lentiviral vectors, as demonstrated by genome quantification (GQA) (FIG. 21A), functional titer (SupT1 IU/mL) (FIG. 218), and calculated particle-to-infectivity ratios (GQA/IU) (FIG. 21C).

FIGS. 22A and 22B show dose titration of the lentiviral vectors used for transducing primary T cells.

FIGS. 23A and 23B show digital-droplet PCR confirmation of the dose titration by measuring vector copy numbers (VCN) integrated within bulk primary t cell pool. FIG. 23B shows deIU3 VCN normalized to CAR positive T cells only.

FIGS. 24A-24J show the cytotoxic effects of hypoimmune CD47-CD19 CAR T cells in NALM-6 and NALM-6 CD19 knockout tumor cells. FIGS. 24A and 24F show NALM-6 and NALM-6 CD19 knockout tumor cell survival, respectively, where the tumor cells were cultured for 24 hours with CD47-CD19 hypoimmune CAR T cells at different effector-target cell ratios. FIGS. 24B-24E show cytokine levels measured by Meso-Scale Discovery (MSD) after culturing of CD47-CD19 hypoimmune CAR T cells with NALM-6 tumor cells. FIGS. 24G-24J show cytokine levels measured after culturing of CD47-CD19 hypoimmune CAR T cells with NALM-6 CD19 knockout tumor cells.

FIGS. 26A-26J show the cytotoxic effects of hypoimmune CD47-CD19 CAR T cells in Raji and Raji CD19 knockout tumor cells. FIGS. 26A and 25F show Raji and Raji CD19 knockout tumor cell survival, respectively, when the tumor cells were cultured for 24 hours with CD47-CD19 hypoimmune CAR T cells at different effector-target cell ratios. FIGS. 268-26E show cytokine levels measured by MSD after culturing of CD47-CD19 hypoimmune CAR T cells with Raji tumor cells. FIGS. 25G-25J show cytokine levels measured after culturing of CD47-CD19 hypoimmune CAR T cells with Raji CD19 knockout tumor cells.

FIGS. 26A-26J show the cytotoxic effects of hypoimmune CD47-CD19 CAR T cells in K562-CD19+ and parental K562 tumor cells. FIGS. 26A and 26F show K562 and K562 CD19 knockout tumor cell survival, respectively, when the tumor cells were cultured for 24 hours with CD47-CD19 hypoimmune CAR T cells at different effector:target cell ratios. FIGS. 26B-26E show cytokine levels measured by MSD after culturing of CD47-CD19 hypoimmune CAR T cells with K562 tumor cells. FIGS. 26G-26J show cytokine levels measured after culturing of CD47-CD19 hypoimmune CAR T cells with K562 CD19 knockout tumor cells.

FIGS. 27A-27F show the cytotoxicity of hypoimmune CD47-CD19 CAR T cells. FIGS. 27A-27C Incucyte analysis of NALM-6 iRFP713+ tumor cell growth when cultured with CD19CAR+ or Mock, unstransduced T cells generated from three different donors. FIGS. 27D-27F show Incucyte analysis of T cell expansion over the course of the study, with hypoimmune CD47-CD19 CAR T cells generated from three different donors when cultured with NALM-6 iRFP713+ tumor cells.

FIGS. 28A-28F show the cytotoxicity of hypoimmune CD47-CD19 CAR T cells. FIGS. 28A-28C incucyte analysis of NALM-6 CD19 knockout iRFP713+ tumor cell growth when cultured with CD19CAR+ or Mock, untransduced T cells generated from three different donors. FIGS. 28D-28F show Incucyte analysis of T cell expansion over the course of the study, with hypoimmune CD47-CD19 CAR T cells generated from three different donors when cultured with NALM-6 CD19KO iRFP713+ tumor cells.

FIGS. 29A-29D show levels of GM-CSF, IFNγ, IL-2, and TNFα, respectively, measured by MSD after from culture supernatant 24 hours after incubation of the hypoimmune CAR T cells with NALM-6 and NALM-6 CD19 knockout cells.

FIGS. 30A-30C show total flux results in the B-cell tumor animal model, where animals received hypoimmune CD47-CD19 CAR T cells generated from three different donors after administration of Nalm6:Wasabi-ffLuc cells.

FIGS. 31A-31C show levels of CAR+ cells in circulating blood on days 13 and 31 in the B-cell tumor model.

FIGS. 32A-32C show median fluorescence intensity (MFI) for CD47 as measured by flow cytometry from cells in circulating blood on days 13 and 31 in the B-cell tumor model.

### DETAILED DESCRIPTION

Unless defined otherwise, all terms of art, notations, and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless defined otherwise, all technical and scientific terms, acronyms, and abbreviations used herein have the same meaning as commonly understood by one of ordinary skill in the art to which the disclosure pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. In some embodiments, the term “about” when referring to a measurable value such as an amount, a temporal duration, and the like, is meant to encompass art-accepted variations based on standard errors in making such measurements. In some embodiments, the term “about” when referring to such values, is meant to encompass variations of ±20% or ±10%, more preferably ±5%, even more preferably ±1%, and still more preferably ±0.1% from the specified value, as such variations are appropriate to perform the disclosed methods.

As used herein, “CD19” or “Cluster of Differentiation 19” refers to a transmembrane glycoprotein which is expressed on cells of 8 cell lineage. CD19 is a marker for B cell development.

As used herein, “CD4” or “cluster of differentiation 4” refers to a transmembrane glycoprotein which is a specific marker for a subclass of T cells (which includes helper T cells). The CD4 protein acts as a co-receptor together with the T cell receptor (TCR) to recognize antigen presentation by MHC class II cells. CD4 plays a role in the development of T cells and activation of mature T cells.

As used herein, “CD8” or “cluster of differentiation 8” refers to a transmembrane glycoprotein which is a specific marker for a subclass of T cells (which includes cytotoxic T cells). CD8 assembles as either a heterodimer of the CD8 alpha (“CD8a” or “CD8A”) and CD8 beta (“CD8β” or “CD8B”) subunits (“CD8ap” or “CD8AB”), or a CD8 alpha homodimer (“CD8aa” or “CD8AA”). The assembled dimeric CD8 complex acts as a co-receptor together with the T cell receptor (TCR) to recognize antigen presentation by MHC class I cells. CD8 plays a role in the development of T cells and activation of mature T cells.

As used herein, “affinity” refers to the strength of the sum total of noncovalent interactions between a single binding site of a molecule (e.g., an antibody) and its binding partner (e.g., an antigen). The affinity of a molecule for its partner can generally be represented by the equilibrium dissociation constant (K_{D}) (or its inverse equilibrium association constant, KA). Affinity can be measured by common methods known in the art, including those described herein. See, for example, Pope M.E., Soste M. V., Eyford B.A., Anderson N. L., Pearson T. W., (2009) J. Immunol. Methods. 341(1-2):86-96 and methods described therein.

As used herein, “antibody” is meant in a broad sense and includes immunoglobulin molecules including monoclonal antibodies including murine, human, humanized and chimeric antibodies, antibody fragments, bispecific or multispecific antibodies formed from at least two intact antibodies or antibody fragments, dimeric, tetrameric or multimeric antibodies, single chain antibodies, and any other modified configuration of the immunoglobulin molecule that comprises an antigen recognition site of the required specificity.

Immunoglobulins can be assigned to five major classes, namely IgA, IgD, IgE, IgG, and IgM, depending on the heavy chain constant domain amino acid sequence. IgA and IgG are further sub-classified to IgA1, IgA2, IgG1, IgG2, IgG3, and IgG4. Antibody light chains of any vertebrate species can be assigned to one of two clearly distinct types, namely kappa (K) and lambda (A), based on the amino acid sequences of their constant domains.

The term “antigen” refers to an immunogenic molecule that provokes an immune response. This immune response involves antibody production, activation of specific immunologically competent cells, or both. An antigen is, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can also be produced by cells that have been modified or genetically engineered to express an antigen.

As used herein, “antigen binding fragment” or “antibody fragment” refers to a portion of an immunoglobulin molecule that retains the heavy chain and/or the light chain antigen binding site, such as a heavy chain complementarity determining regions (HCDR) 1 (HCDR1), 2 (HCDR2), and 3 (HCDR3), a light chain complementarity determining regions (LCDR) 1 (LCDR1), 2 (LCDR2), and 3 (LCDR3), a heavy chain variable region (VH), or a light chain variable region (VL). Antibody fragments include a Fab fragment (a monovalent fragment consisting of the VL or the VH); a F(ab) 2 fragment (a bivalent fragment comprising two Fab fragments linked by a disulfide bridge at the hinge region); a Fd fragment consisting of the VH and CH1 domains; a Fv fragment consisting of the VL and VH domains of a single arm of an antibody; a dAb fragment, which consists of a VH domain; and a variable domain (VHH) from, e.g., human or camelid origin. VH and VL domains are engineered and linked together via a synthetic linker to form various types of single chain antibody designs in which the VH/VL domains pair intramolecularly, or intermolecularly in those embodiments in which the VH and VL domains are expressed by separate single chain antibody constructs, to form a monovalent antigen binding site, such as a single-chain Fv (scFv) or diabody. These antibody fragments are obtained using well known techniques and the fragments are characterized in the same manner as are intact antibodies.

An antibody variable region consists of a “framework” region interrupted by three “antigen binding sites.” The antigen binding sites are defined using various terms, including, for example (i) “Complementarity Determining Regions” (CDRs), three in the VH (HCDR1, HCDR2, HCDR3) and three in the VL (LCDR1, LCDR2, LCDR3) (Wu and Kabat, J Exp Med 132:211-50. 1970; Kabat et al., Sequences of Proteins of Immunological Interest, 5th Ed. Public Health Service, National Institutes of Health, Bethesda, Md., 1991), and (ii) “Hypervariable regions,” “HVR,” or “HV,” three in the VH (H1, H2. H3) and three in the VL (L1, 12, 13) (Chothia and Lesk Mol Biol 196:901-17, 1987). Other terms include “IMGT-CDRs” (Lefranc et al., Dev Comparat Immunol27:55-77, 2003) and “Specificity Determining Residue Usage” (SDRU) (Almagro Mol Recognit, 17:132-43, 2004). The International ImMunoGeneTics (IMGT) database (http://www_imgt org) provides a standardized numbering and definition of antigen-binding sites. The correspondence between CDRs, HVs, and IMGT delineations is described in Lefranc et al., Dev Comparat Immunol 27:55-77, 2003.

The term “framework,” or “FR” or “framework sequence” refers to the remaining sequences of a variable region other than those sequences defined to be antigen binding sites. Because the antigen binding site can be defined by various terms as described above, the exact amino acid sequence of a framework depends on how the antigen-binding site was defined.

A “binding domain,” also referred to as a “binding region,” refers to an antibody or portion thereof that possesses the ability to specifically and non-covalently associate, unite, or combine with a target. A binding domain includes any naturally occurring, synthetic, semi-synthetic, or recombinantly produced binding partner for a biological molecule, a molecular complex, or other target of interest. Exemplary binding domains include receptor ectodomains, ligands, scFvs, disulfide linked Fvs, sdAbs, VHH antibodies, Fab fragments, Fab′ fragments, F(ab′)2 fragments, diabodies, or other synthetic polypeptides selected for their specific ability to bind to a biological molecule, a molecular complex, or other target of interest.

The term “CDR” denotes a complementarity determining region as defined by at least one manner of identification to one of skill in the art. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service. National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,* J Mol Biol, 2001 Jun. 8; 309(3):657-70, (“Aho” numbering scheme); and Martin et at., “Modeling antibody hypervariable loops: a combined algorithm,” PNAS, 1989, 86(23):9268-9272, (AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

In some embodiments, CDRs can be defined in accordance with any of the Chothia numbering schemes, the Kabat numbering scheme, the IMGT numbering scheme, a combination of Kabat, IMGT, and Chothia, the AbM definition, and/or the contact definition. A sdAb variable domain comprises three CDRs, designated CDR1, CDR2, and CDR3. Table 1 lists exemplary position boundaries of CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-H1 located before CDR-H1, FR-H2 located between CDR-H1 and CDR-H2, FR-H3 located between CDR-H2 and CDR-H3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given sdAb amino acid sequence, it is understood that such a CDR has a sequence of the corresponding CDR (e.g., CDR-H3) within the sdAb, as defined by any of the aforementioned schemes. It is understood that any antibody, such as a sdAb, includes CDRs and such are identified according to any of the other aforementioned numbering schemes or other numbering schemes known to a skilled artisan.

As used herein, “Fv” refers to the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) may have the ability to recognize and bind an antigen, although at a lower affinity than the entire binding site.

As used herein, “single-chain Fv” or “scFv” antibody fragments comprise the VH and VL domains of an antibody, wherein these domains are present in a single polypeptide chain. Preferably, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

As used herein, “VHH” or “VHH antibodies” refer to single domain antibodies that consist of the variable region of a heavy chain of an IgG antibody. For example, the terms “VHH” and “VHH antibody” can refer to the antigen binding domain of a heavy chain IgG (hcIgG) molecule produced by a Camelidae family mammal (e.g., llamas, camels, and alpacas).

As used herein, the term “specifically binds” to a target molecule, such as an antigen, means that a binding molecule, such as a single domain antibody (sdAb), reacts or associates more frequently, more rapidly, with greater duration, and/or with greater affinity with a particular target molecule than it does with alternative molecules. A binding molecule, such as a sdAb or scFv, specifically binds toa target molecule if it binds with greater affinity, avidity, more readily, and/or with greater duration than it binds to other molecules. It is understood that a binding molecule, such as a sdAb or scFv, that specifically binds to a first target may or may not specifically bind to a second target. As such, “specific binding” does not necessarily require (although it can include) exclusive binding.

As used herein, the term “cell surface molecule” means a molecule that is present on the outer surface of a cell. In some embodiments, the cell surface molecule is an antigen, as herein defined and disclosed. In some embodiments, the cell surface molecule is, for example, a peptide, glycopeptide, polypeptide, glycopolypeptide, polynucleotide, polysaccharide, lipid, or the like that is not immunogenic.

As used herein, “percent (%) amino acid sequence identity” and “homology” with respect to a peptide, polypeptide or antibody sequence are used interchangeably and are defined as the percentage of amino acid residues in a candidate sequence that are identical with the amino acid residues in another peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNASTAR) software.

Those skilled in the art can determine appropriate parameters for measuring alignment, including any algorithms needed to achieve maximal alignment over the full length of the sequences being compared.

An amino acid substitution may include but is not limited to the replacement of one amino acid in a polypeptide with another amino acid. Exemplary substitutions are shown in Table 2. Amino acid substitutions are introduced into an antibody of interest and the products screened for a desired activity, for example, retained/improved binding.

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. The term, “corresponding to” with reference to nucleotide or amino acid positions of a sequence, such as set forth in the Sequence Listing, refers to nucleotides or amino acid positions identified upon alignment with a target sequence based on structural sequence alignment or using a standard alignment algorithm, such as the GAP algorithm. For example, corresponding residues of a similar sequence (e.g., fragment or species variant) can be determined by alignment to a reference sequence by structural alignment methods. By aligning the sequences, one skilled in the art can identify corresponding residues, for example, using conserved and identical amino acid residues as guides.

The term “construct” refers to any polynucleotide that contains a recombinant nucleic acid molecule. A construct is present in a vector (e.g., a bacterial vector, a viral vector) or is integrated into a genome. A “vector” is a nucleic acid molecule that is capable of introducing a specific nucleic acid sequence into a cell or into another nucleic acid sequence, or as a means of transporting another nucleic acid molecule. Vectors are, for example, plasmids, cosmids, viruses, an RNA vector, or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “polypeptide” refers to a polymer comprising amino acids that are linked together. In some embodiments, a polypeptide is a linear polymer of nucleic acids in a chain. In some embodiments, a polypeptide is a polymer of nucleic acids that is folded into a structure or shape.

The term “hypoimmunogenicity,” “hypoimmunogenic,” “hypoimmunogenic,” “hypoimmunity,” or “hypoimmune” is used interchangeably to describe a cell being less prone to immune rejection by a subject into which such cell is transplanted. For example, relative to an unaltered or unmodified wild-type cell, such a hypoimmunogenic cell is about 2.5%, 5%, 10%, 20%, 30%, 40%, 50%, 60%, 70%, 80%, 90%, 95%, 97.5%, 99% or more less prone to immune rejection by a subject into which such cell is transplanted. In some examples described herein, genome editing technologies are used to modulate the expression of MHC I and/or MHC 11 genes, and thus, to generate a hypoimmunogenic cell. In other examples described herein, a tolerogenic factor is introduced into a cell and when expressed can modulate or affect the ability of the cell to be recognized by host immune system and thus confer hypoimmunogenicity. Hypoimmunogenicity of a cell is determined by evaluating the cell's ability to elicit adaptive and innate immune responses. Such immune response can be measured using assays recognized by those skilled in the art, for example, by measuring the effect of a hypoimmunogenic cell on T cell proliferation, T cell activation, T cell killing, NK cell proliferation, NK cell activation, and macrophage activity. Hypoimmunogenic cells may undergo decreased killing by T cells and/or NK cells upon administration to a subject or show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

The term “isolated” as used herein refers to a molecule that has been separated from at least some of the components with which it is typically found in nature or produced. For example, a polypeptide is referred to as “isolated” when it is separated from at least some of the components of the cell in which it was produced. When a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced. Thus, a DNA polynucleotide that is contained in a vector inside a host cell is referred to as “isolated.”

As used herein, “lipid particle” refers to any biological or synthetic particle that contains a bilayer of amphipathic lipids enclosing a lumen or cavity. Typically, a lipid particle does not contain a nucleus. Examples of lipid particles include nanoparticles, viral-derived particles, or cell-derived particles. Such lipid particles include, but are not limited to, viral particles (e.g., lentiviral particles), virus-like particles, viral vectors (e.g., lentiviral vectors), exosomes, enucleated cells, vesicles (e.g., microvesicles, membrane vesicles, extracellular membrane vesicles, plasma membrane vesicles, and giant plasma membrane vesicles), apoptotic bodies, mitoparticles, pyrenocytes, or lysosomes. In some embodiments, a lipid particle is a fusosome. In some embodiments, the lipid particle is not a platelet.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. In some embodiments, the retained activity includes 10%-150% or more of the activity of a full-length or wild-type F protein or G protein. Examples of biologically active portions of F and G proteins include truncations of the cytoplasmic domain, e.g., truncations of up to 1, 2, 3, 4, 5, 6, 7, 8 9, 10, 11, 12, 13, 14, 15, 20, 22, 25, 30, 33, 34, 35, or more contiguous amino acids, see e.g. Khetawat and Broder 2010 Virology Journal 7:312: Witting et al. 2013 Gene Therapy 20:997-1005; published international; patent application No. WO/2013/148327.

As used herein, “G protein” refers to a henipavirus envelope attachment glycoprotein G or biologically active portion thereof. “F protein” refers to a henipavirus fusion protein F or biologically active portion thereof. In some embodiments, the F and G proteins are from a Hendra (HeV) or a Nipah (NiV) virus, and are a wild-type protein or are a variant thereof that exhibits reduced binding for the native binding partner. The F (fusion) and G (attachment) glycoproteins mediate cellular entry of Nipah virus. The G protein initiates infection by binding to the cellular surface receptor ephrin-82 (EphB2) or Eph83. The subsequent release of the viral genome into the cytoplasm is mediated by the action of the F protein, which induces the fusion of the viral envelope with cellular membranes. In some embodiments, the efficiency of transduction of targeted lipid particles is improved by engineering hyperfusogenic mutations in one or both of the F protein (such as NiV-F) and G protein (such as NiV-G).

As used herein, “fusosome” refers to a particle containing a bilayer of amphipathic lipids enclosing a lumen or cavity and a fusogen that interacts with the amphipathic lipid bilayer. In some embodiments, the fusosome comprises a nucleic acid. In some embodiments, the fusosome is a membrane enclosed preparation. In some embodiments, the fusosome is derived from a source cell. As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain.

As used herein, a “re-targeted fusogen” refers to a fusogen that comprises a targeting moiety having a sequence that is not part of the naturally-occurring form of the fusogen. In embodiments, the fusogen comprises a different targeting moiety relative to the targeting moiety in the naturally-occurring form of the fusogen. In embodiments, the naturally occurring form of the fusogen lacks a targeting domain, and the re-targeted fusogen comprises a targeting moiety that is absent from the naturally occurring form of the fusogen. In embodiments, the fusogen is modified to comprise a targeting moiety. In embodiments, the fusogen comprises one or more sequence alterations outside of the targeting moiety relative to the naturally occurring form of the fusogen, e.g., in a transmembrane domain, fusogenically active domain, or cytoplasmic domain.

As used herein, a “targeted envelope protein” refers to a polypeptide that contains a henipavirus G protein (G protein) attached to a single domain antibody (sdAb) variable domain, such as a VL or VH sdAb, a scFv, a nanobody, a camelid VHH domain, a shark IgNAR, or fragments thereof, that target a molecule on a desired cell type. In some such embodiments, the attachment is directly or indirectly via a linker, such as a peptide linker. The “targeted envelope protein” may also be referred to as a “fusion protein” comprising the G protein and antibodies or antigen binding fragments of the disclosure in which the antibody or antigen binding fragment is fused to the C-terminus of the G protein or a biologically active portion thereof.

As used herein, a “targeted lipid particle” refers to a lipid particle that contains a targeted envelope protein embedded in the lipid bilayer, e.g., a targeted envelope protein targeting CD4 or CD8. Such targeted lipid particles are any lipid particle as is herein disclosed, e.g., a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP), or a cell derived particle.

As used herein, a “retroviral nucleic acid” refers to a nucleic acid containing at least the minimal sequence requirements for packaging into a retrovirus or retroviral vector, alone or in combination with a helper cell, helper virus, or helper plasmid. In some embodiments, the retroviral nucleic acid further comprises or encodes an exogenous agent, a positive target cell-specific regulatory element, a non-target cell-specific regulatory element, or a negative TCSRE. In some embodiments, the retroviral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Ψ)), and RRE (e.g., to bind to Rev and promote nuclear export). The retroviral nucleic acid can comprise RNA (e.g., when part of a virion) or DNA (e.g., when being introduced into a source cell or after reverse transcription in a recipient cell). In some embodiments, the retroviral nucleic acid is packaged using a helper cell, helper virus, or helper plasmid which comprises one or more of (e.g., all of) gag, pol, and env.

As used herein, a “target cell” refers to a cell of a type to which it is desired that a targeted lipid particle delivers an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., a re-targeted fusogen, leads to preferential delivery of the exogenous agent to a target cell compared to a non-target cell.

As used herein a “non-target cell” refers to a cell of a type to which it is not desired that a targeted lipid particle delivers an exogenous agent. In some embodiments, a non-target cel is a cell of a specific tissue type or class. In some embodiments, a non-target cell is a non-diseased cell, e.g., a non-cancerous cell. In some embodiments, the fusogen, e.g., a re-targeted fusogen, leads to lower delivery of the exogenous agent to a non-target cell compared to a target cell.

The term “effective amount” as used herein means an amount of a pharmaceutical composition which is sufficient to significantly and positively modify the symptoms and/or conditions to be treated (e.g., provide a positive clinical response). The effective amount of the targeted lipid particles of the disclosure for use in a pharmaceutical composition will vary with the particular condition being treated, the severity of the condition, the duration of treatment, the nature of concurrent therapy, the particular lipid particle) being employed, the particular pharmaceutically-acceptable excipient(s) and/or carrier(s) utilized, and like factors with the knowledge and expertise of the attending physician.

An “exogenous agent” as used herein with reference to a targeted lipid particle, refers to an agent that is neither comprised by nor encoded in the corresponding wild-type virus or fusogen made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises DNA, RNA, or protein.

As used herein, the term “operably linked” refers to the association of two or more nucleic acid molecules on a single nucleic acid fragment so that the function of one is affected by the other.

As used herein, “nucleic acid” or “polynucleotide” refers to a polymeric compound including covalently linked nucleotides comprising natural subunits (e.g., purine or pyrimidine bases). In some embodiments, a polynucleotide comprises a transgene. Purine bases include adenine and guanine, and pyrimidine bases include uracil, thymine, and cytosine. Nucleic acid molecules include ribonucleic acid (RNA) and deoxyribonucleic acid (DNA), which includes cDNA, genomic DNA, and synthetic DNA, either of which are single- or double-stranded. A nucleic acid molecule encoding an amino acid sequence includes all nucleotide sequences that encode the same amino acid sequence.

As used herein, a “transgene” refers to genetic material that has been transferred to a cell (e.g., a host cell). A transgene comprises nucleic acids, and is, in some embodiments, incorporated into a cell through any of the methods disclosed herein.

As used herein, a “promoter” refers to a cis-regulatory DNA sequence that, when operably linked to a gene coding sequence, drives transcription of the gene. The promoter may comprise one or more transcription factor binding sites. In some embodiments, a promoter works in concert with one or more enhancers which are distal to the gene.

The term “safe harbor locus” refers to a gene locus that allows safe expression of a transgene or an exogenous gene. Safe harbors or genomic safe harbors are sites in the genome able to accommodate the integration of new genetic material in a manner that permits the newly inserted genetic elements to: (i) function predictably and (ii) do not cause alterations of the host genome posing a risk to the host cell or organism. Exemplary “safe harbor” loci include a CCR5 gene, a CXCR4 gene, a PPP1R12C (also known as AAVS1) gene, an albumin gene, and a Rosa gene.

The term “safety switch” refers to a system for controlling the expression of a gene or protein of interest that, when downregulated or upregulated, leads to clearance or death of the cell, e.g., through recognition by the host's immune system. A safety switch is designed to be or include an exogenous molecule administered to prevent or mitigate an adverse clinical event. A safety switch is engineered by regulating the expression on the DNA, RNA and protein levels. A safety switch may include a protein or molecule that allows for the control of cellular activity in response to an adverse event. In some embodiments, a safety switch refers to an agent (e.g., protein, molecule, etc.) that binds a specific cel and targets it for cell death or elimination. In some instances, the safety switch is a blockade agent that binds a target protein on the surface of a cell, which in turn, triggers an immune response. In one embodiment, the safety switch is a “kill switch” that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis.

The term “tolerogenic factor” as used herein includes hypoimmunity factors, complement inhibitors, and other factors that modulate or affect (e.g., reduce) the ability of a ce to be recognized by the immune system of a host or recipient subject upon administration, transplantation, or engraftment. Tolerogenic factors include but are not limited to CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, Serpinb9, CC121, Mfge8, A20/TNFAIP3, CCL21, CD16 Fc receptor, CD27, CR1, DUX4, H2-M3 (HLA-G), HLA-F, IL15-RF, MANF, IL-39, and B2M-HLA-E.

As used herein, a composition refers to any mixture of two or more products, substances, or compounds, including cells. It includes a solution, a suspension, liquid, powder, a paste, aqueous, non-aqueous, or any combination thereof.

As used herein, the term “pharmaceutically acceptable” refers to a material, such as a carrier or diluent, which does not abrogate the biological activity or properties of a therapeutic compound, and is relatively nontoxic, i.e., the material is administered to an individual without causing undesirable biological effects or interacting in a deleterious manner with any of the components of the composition in which it is contained.

As used herein, the term “pharmaceutical composition” refers to a mixture of at least one targeted lipid particle of the disclosure with other chemical components, such as carriers, stabilizers, diluents, dispersing agents, suspending agents, thickening to agents, and/or excipients. The pharmaceutical composition facilitates administration of the targeted lipid particle to an organism. Multiple techniques of administering targeted lipid particles of the disclosure exist in the art including, but not limited to, intravenous, oral, aerosol, parenteral, ophthalmic, pulmonary, and topical administration.

A “disease” or “disorder” as used herein refers to a condition in which treatment is needed and/or desired.

As used herein, the terms “treat.” “treating.” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder Includes obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example, a mammal. The terms include human and veterinary animals. In some embodiments, methods of treating animals, including, but not limited to, humans, rodents, simians, felines, canines, equines, bovines, porcines, ovines, caprines, mammalian laboratory animals, mammalian farm animals, mammalian sport animals, and mammalian pets, are provided. The animal is male or female and is any suitable age, including infant, juvenile, adolescent, adult, and geriatric. In some examples, an “individual” or “subject” refers to an animal in need of treatment for a disease or disorder. In some embodiments, the animal to receive the treatment is a “patient,” designating the fact that the animal has been identified as having a disorder of relevance to the treatment, or being at adequate risk of contracting the disorder. In some embodiments, the animal is a human, such as a human patient.

The terms “treat,” “treating,” and “treatment” as used herein with regard to cancer refers to alleviating the cancer partially or entirely; preventing the cancer; decreasing the likelihood of occurrence or recurrence of the cancer; slowing the progression or development of the cancer; eliminating, reducing, or slowing the development of one or more symptoms associated with the cancer; or increasing progression-free or overall survival of the cancer. For example, “treating” may refer to preventing or slowing the existing cancer from growing larger; preventing or slowing the formation or metastasis of cancer, and/or slowing the development of certain symptoms of the cancer. In some embodiments, the term “treat,” “treating,” or “treatment” means that the subject has a reduced number or size of cancer cells comparing to a subject without being administered with the treatment. In some embodiments, the term “treat,” “treating,” or “treatment” means that one or more symptoms of the cancer are alleviated in a subject receiving the treatment as disclosed and described herein comparing to a subject who does not receive such treatment.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the technology described herein is not entitled to antedate such publication by virtue of prior technology. Further, the dates of publication provided might be different from the actual publication dates, which may need to be independently confirmed.

Before the technology is further described, it is to be understood that this technology is not limited to particular embodiments described, as such may, of course, vary. It is also to be understood that the terminology used herein is for the purpose of describing particular embodiments only, and is not intended to be limiting, since the scope of the present disclosure will be limited only by the appended claims. It should also be understood that the headers used herein are not limiting and are merely intended to orient the reader, but the subject matter generally applies to the technology disclosed herein.

### CD19-Specific Polypeptides

Described herein are novel polypeptides that specifically target and bind human CD19. In some embodiments, the polypeptides may cross-react with cynomolgus (or “cyno”) or M. nemestrina CD19. In some embodiments, the polypeptides are antibodies or antigen binding fragments thereof. The present disclosure also provides polynucleotides encoding the polypeptides, vectors, and host cells, and methods of using the polypeptides thereof. In some embodiments, e.g., the polypeptides are fused to henipavirus glycoprotein G for targeted binding and transduction to cells. In some embodiments, the polypeptide comprises an antigen binding region that specifically binds CD19.

Sequences for exemplary polypeptides of the disclosure comprising antigen binding regions using the Kabat numbering scheme are shown in Tables 3-4 below. In some embodiments, the antigen binding regions comprises one or more heavy chain complementarity determining regions (HCDRs). In some embodiments, the antigen binding regions comprises one or more light chain complementarity determining regions (LCDRs). In some embodiments, the antigen binding regions comprise a heavy chain variable region (VH). In some embodiments, the antigen binding regions comprise a light chain variable region (VL). Sequences for exemplary HCDRs of the disclosure are shown in Table 3. Sequences for exemplary LCDRs of the disclosure are shown in Table 4.

The sequences for the disclosed VH and VL domains are provided in Tables 5-6. Tables 7-10 provided herein show the CDR sequences of the disclosed polypeptides thereof using both Chothia and IMGT numbering schemes.

In some embodiments, a polypeptide capable of binding CD19 is disclosed. In some embodiments, the polypeptide comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3). In some embodiments, the HCDR1, HCDR2, and HCDR3 comprise amino acid sequences of any one of the SEQ ID NOs recited in Tables 3, 7, and 9, and the LCDR1, LCDR2, and LCDR3 comprise amino acid sequences of any one of the SEQ ID NOs recited in Tables 4, 8, and 10. In some embodiments, the heavy chain variable region (VH) comprises an amino acid sequence of any one of SEQ ID NOs: 19-21 (Table 5) and the light chain variable region (VL) comprises an amino acid sequence of any one of SEQ ID NOs: 22-24 (Table 6).

In some embodiments, the polypeptide comprises an amino acid sequence with at least 80%, 85%, 90%, 95%, 96%, 97%, 98%, 99%, or 100% identity to a sequence selected from SEQ ID NOs: 19-21.

In some embodiments, the polypeptide comprises an amino acid sequence with at least 80%, 85%, 90%, 95%, 96%, 97%, 98%, 99%, or 100% identity to a sequence selected from SEQ ID NOs: 22-24.

In some embodiments, the polypeptide comprises an amino acid sequence with at least 80%, 85%, 90%, 95%, 96%, 97%, 98%, 99%, or 100% identity to a sequence selected from SEQ ID NOs: 19-21 and an amino acid sequence with at least 80%, 85%, 90%, 95%, 96%, 97%, 98%, 99%, or 100% identity to a sequence selected from SEQ ID NOs: 22-24.

In some embodiments, the polypeptide comprises the amino acid sequence of SEQ ID NOs: 1, 4, 7, 10, 13, and 16.

In some embodiments, the polypeptide comprises the amino acid sequence of SEQ ID NOs: 2, 5, 8, 11, 14, and 17.

In some embodiments, the polypeptide comprises the amino acid sequence of SEQ ID NOs: 3, 6, 9, 12, 15, and 18.

In some embodiments, the polypeptide is an antibody or antigen binding fragment thereof as disclosed herein.

Polypeptides whose amino acid sequences differ insubstantially from those shown in Tables 3-6 are encompassed within the scope of the disclosure. Typically, this involves one or more conservative amino acid substitutions with an amino acid having similar charge, hydrophobic, or stereo chemical characteristics in the antigen-binding site or in the framework without adversely altering the properties of the polypeptide. Conservative substitutions may also be made to improve polypeptide properties, for example stability or affinity, 1, 2, 3, 4, 5, 8, 7, 8, 9, 10, 11, 12, 13, 14, or 15 amino acid substitutions are made to the amino acid sequence. For example, a “conservative amino acid substitution” may involve a substitution of a native amino acid residue with a nonnative residue such that there is little or no effect on the polarity or charge of the amino acid residue at that position. Desired amino acid substitutions are determined by those skilled in the art at the time such substitutions are desired. For example, amino acid substitutions are used to identify important residues of the molecule sequence, or to increase or decrease the affinity of the molecules described herein. The following eight groups contain amino acids that are conservative amino acid substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (1), Leucine (L), Methionine (M), Valine (V); 6) Phenylalanine (F), Tyrosine (Y), Tryptophan (W); 7) Serine (S), Threonine (T); and 8) Cysteine (C), Methionine (M).

In some embodiments, the polypeptide binds to human CD19. In some embodiments, the polypeptide is an antibody or antigen binding fragment binding that specifically binds CD19 as disclosed herein.

In some embodiments, the polypeptide binds to human CD19 with an affinity constant (K_{D}) of between about 1 nM and about 900 nM. In some embodiments, the K_{D} to human CD19 is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the polypeptide binds to human CD19 with an affinity constant (K_{D}) of 500 nM, 400 nM, 300 nM, 200 nM, 100 nM, 90 nM, 80 nM, 70 nM, 60 nM, 50 nM, 40 nM, 30 nM, 20 nM, or 10 nM or lower. In some embodiments, the polypeptide binds to human CD19 and cynomolgus, M. mulatta (rhesus monkey), or M. nemestrina CD19 with comparable binding affinity (K_{D}).

In some embodiments, the polypeptide binds to cynomolgus, M. mulatta (rhesus monkey), or N. nemestrina CD19. In some embodiments, the polypeptide binds to mouse, dog, pig, etc., CD19. In some embodiments, the K_{D} to cynomolgus or M. nemestrina CD19 is between about 5 nM about 500 nM, about 6 nM to about 10 nM, about 11 nM to about20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the polypeptide binds to cynomolgus or M. nemestrina CD19 with an affinity constant (K_{D}) of 500 nM, 400 nM, 300 nM, 200 nM, 100 nM, 90 nM, 80 nM, 70 nM, 60 nM, 50 nM, 40 nM, 30 nM, 20 nM, or 10 nM or lower.

A polypeptide that specifically binds CD19 refers to a polypeptide that preferentially binds to CD19, respectively, over other antigen targets. As used herein, the term is interchangeable with an “anti-CD19” polypeptide or an “polypeptide that binds CD19.” In some embodiments, the polypeptide capable of binding to CD19 can do so with higher affinity for that antigen than others. In some embodiments, polypeptide capable of binding CD19 can bind to that antigen with a K_{D} of at least about 10⁻¹, 10⁻², 10⁻³, 10⁻⁴, 10⁻⁵, 10 ⁻⁶, 10⁻⁷, 10⁻⁸, 10⁻⁹, 10⁻¹⁰, 10⁻¹¹, 10⁻¹² or greater (or any value in between). e.g., as measured by surface plasmon resonance or other methods known to those skilled in the art.

In some embodiments, the polypeptide is bispecific. In some embodiments, the bispecific polypeptide comprises an antigen binding region that specifically binds CD19 as herein disclosed and an antigen binding region that specifically binds CD3, 4-18B, IL-6, NKG2D, Fc-gamma-RIIIA (CD16), APRIL, CD38, TACI, Fc-gamma-RIIIA (CD16) and NKG2D, CD3 and serum albumin, CD47 and TACI, or CD3 and GPRCSD. In some embodiments, the antigen binding region comprises an antibody or antibody binding fragment thereof.

In some embodiments, the polypeptide is conjugated. In some embodiments, the polypeptide is a polypeptide-drug conjugate, wherein the polypeptide that specifically binds CD19 as herein disclosed is conjugated to a therapeutic agent or diagnostic agent. In some embodiments, the polypeptide is conjugated to a tag for detection. In some embodiments, the polypeptide is conjugated to a conjugate that enhances polypeptide stability. In some embodiments, the polypeptide is conjugated to a cleavable linker, wherein the linker allows for another molecule to be conjugated to the polypeptide. In some embodiments, the polypeptide is conjugated to a nanoparticle.

Some embodiments of the disclosure are an isolated polynucleotide encoding any of the polypeptides of the disclosure. Certain exemplary polynucleotides are disclosed herein, however, other polynucleotides which, given the degeneracy of the genetic code or codon preferences in a given expression system, encode the polypeptides of the disclosure are also within the scope of the disclosure. The polynucleotide sequences encoding an antigen binding region thereof of the polypeptide of the disclosure are operably linked to one or more regulatory elements, such as a promoter and enhancer, that allow expression of the nucleotide sequence in the intended host cell. In some embodiments, the polynucleotide is a cDNA.

Some embodiments of the disclosure are a vector comprising the polynucleotide of the disclosure. In some embodiments, such vectors are plasmid vectors, viral vectors, vectors for baculovirus expression, transposon-based vectors, or any other vector suitable for introduction of the polynucleotide of the disclosure into a given organism or genetic background by any means. In some embodiments, the vector is polycistronic. For example, polynucleotides encoding light and heavy chain variable regions of the polypeptide of the disclosure, optionally linked to constant regions, are inserted into expression vectors. The light and heavy chains are cloned in the same or different expression vectors. The DNA segments encoding immunoglobulin chains are operably linked to control sequences in the expression vector(s) that ensure the expression of immunoglobulin polypeptides. Such control sequences include signal sequences, promoters (e.g., naturally associated or heterologous promoters), enhancer elements, and transcription termination sequences, and are chosen to be compatible with the host cell chosen to express the polypeptide. Once the vector has been incorporated into the appropriate host, the host is maintained under conditions suitable for high level expression of the polypeptides encoded by the incorporated polynucleotides.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance, or neomycin resistance to permit detection of those cells transformed with the desired DNA sequences. Suitable vectors, promoter, and enhancer elements are known in the art; many are commercially available for generating subject recombinant constructs.

Some embodiments of the disclosure are a host cell comprising the vector of the disclosure. The term “host cell” refers to a cell into which a vector has been introduced. It is understood that the term host cell is intended to refer not only to the particular subject call but to the progeny of such a cell. Because certain modifications may occur in succeeding generations due to either mutation or environmental influences, such progeny may not be identical to the parent cell, but are still included within the scope of the term “host cell” as used herein. Such host cells include eukaryotic cells, prokaryotic cells, plant cells, or archaeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells include cells of mammalian, insect, avian, or other animal origins.

### CD19-Specific Antibodies

Described herein are novel antibodies and antigen binding fragments thereof that specifically target and bind human CD19. In some embodiments, the antibodies or antigen binding fragments thereof may cross-react with cynomolgus (or “cyno”) or M. nemestrina CD19. In some embodiments, the antibodies or antigen binding fragments thereof are single-chain variable fragments (scFvs) composed of the antigen-binding domains derived from the heavy (VH) and the light (VL) chains of the IgG molecule and connected via a linker domain. In some embodiments, the antibodies or antigen binding fragments are single domain antibodies (sdAbs) composed of the antigen-binding domain derived from a heavy (VH) or light (VL) chain of the IgG molecule. In some embodiments, the antibodies or antigen binding fragments thereof are VHHs that correspond to the VH of the IgG molecule. The present disclosure also provides polynucleotides encoding the antibodies and fragments thereof, vectors, and host cells, and methods of using the antibodies or antigen binding fragments thereof. In some embodiments, e.g., the antibodies or antigen binding fragments thereof are fused to henipavirus glycoprotein G for targeted binding and transduction to cells.

Sequences for exemplary antibodies and antigen binding fragments of the disclosure using the Kabat numbering scheme are shown in Tables 3-4 below. Sequences for exemplary HCDRs of the disclosure are shown in Table 3. Sequences for exemplary LCDRs of the disclosure are shown in Table 4.

The sequences for the disclosed VH and VL domains are provided in Tables 5-6. Tables 7-10 provided herein show the CDR sequences of the disclosed antibodies and antigen binding fragments thereof using both Chothia and IMGT numbering schemes. The full CD19 binder sequences of the variant CD19 scFvs and VHHs of the disclosure are shown in Table 11.

In some embodiments, an antibody or antigen binding fragment thereof capable of binding CD19 is disclosed, comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3). In some embodiments, the HCDR1, HCDR2, and HCDR3 comprise amino acid sequences of any one of the SEQ ID NOs recited in Tables 3, 7, and 9, and the LCDR1, LCDR2, and LCDR3 comprise amino acid sequences of any one of the SEQ ID NOs recited in Tables 4, 8, and 10. In some embodiments, the heavy chain variable region (VH) comprises an amino acid sequence of any one of SEQ ID NOs: 19-21 (Table 5) and the light chain variable region (VL) comprises an amino acid sequence of any one of SEQ ID NOs: 22-24 (Table 6).

In some embodiments, the antibody or antigen binding fragment thereof comprises a VH having an amino acid sequence with at least 80%, 85%, 90%, 95%, 96%, 97%, 98%, 99%, or 100% identity to a sequence selected from SEQ ID NOs: 19-21.

In some embodiments, the antibody or antigen binding fragment thereof comprises a VL having an amino acid sequence with at least 80%, 85%, 90%, 95%, 96%, 97%, 98%, 99%, or 100% identity to a sequence selected from SEQ ID NOs: 22-24.

In some embodiments, the antibody or antigen binding fragment comprises a VH having an amino acid sequence with at least 80%, 85%, 90%, 95%, 96%, 97%, 98%, 99%, or 100% identity to a sequence selected from SEQ ID NOs: 19-21 and a VL having an amino acid sequence with at least 80%, 85%, 90%, 95%, 96%, 97%, 98%, 99%, or 100% identity to a sequence selected from SEQ ID NOs: 22-24.

In some embodiments, the antibody or antigen binding fragment thereof comprises the HCDR1, HCDR2, HCDR3, LCDR1, LCDR2, and LCDR3 of SEQ ID NOs: 1, 4, 7, 10, 13, and 16, respectively.

In some embodiments, the antibody or antigen binding fragment thereof comprises the HCDR1, HCDR2, HCDR3, LCDR1. LCDR2, and LCDR3 of SEQ ID NOs: 2, 5, 8, 11, 14, and 17, respectively.

In some embodiments, the antibody or antigen binding fragment thereof comprises the HCDR1, HCDR2, HCDR3, LCDR1, LCDR2, and LCDR3 of SEQ ID NOs: 3,6. 9, 12, 15, and 18, respectively.

In some embodiments, the single domain antibody is human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In various embodiments, any of the antibodies or antigen binding fragments described herein can comprise a heavy chain constant region and a light chain constant region. In some embodiments, the heavy chain constant region is an IgG, IgM, IgA, IgD, or IgE isotype, or a derivative or fragment thereof that retains at least one effector function of the intact heavy chain. In some embodiments, the heavy chain constant region is a human IgG isotype. In some embodiments, the heavy chain constant region is a human IgG1 or human IgG4 isotype. In some embodiments, the heavy chain constant region is a human IgG1 isotype. In some embodiments, the light chain constant region is a human kappa light chain or lambda light chain or a derivative or fragment thereof that retains at least one effector function of the intact light chain. In some embodiments, the light chain constant region is a human kappa light chain.

In various embodiments, any of the disclosed antibodies or antigen binding fragments are a rodent antibody or antigen binding fragment thereof, a chimeric antibody or an antigen binding fragment thereof, a CDR-grafted antibody or an antigen binding fragment thereof, or a humanized antibody or an antigen binding fragment thereof. In some embodiments, any of the disclosed antibodies or antigen binding fragments comprises human or human-derived heavy and light chain variable regions. including human frameworks or human frameworks with one or more backmutations. In various embodiments, any of the disclosed antibodies or antigen binding fragments are a Fab, Fab′, F(ab′)2, Fd, scFv, (scFv)2, scFv-Fc, VHH, or Fv fragment.

Antibodies whose heavy chain CDR, light chain CDR. VH, or VL amino acid sequences differ insubstantially from those shown in Tables 3-8 are encompassed within the scope of the disclosure. Typically, this involves one or more conservative amino acid substitutions with an amino acid having similar charge, hydrophobic, or stereo chemical characteristics in the antigen-binding site or in the framework without adversely altering the properties of the antibody. Conservative substitutions may also be made to improve antibody properties, for example stability or affinity. 1, 2, 3, 4, 5, 6, 7, 8, 9, 10, 11, 12, 13, 14, or 15 amino acid substitutions are made to the VH or VL sequence. For example, a “conservative amino acid substitution” may involve a substitution of a native amino acid residue with a nonnative residue such that there is little or no effect on the polarity or charge of the amino acid residue at that position. Desired amino acid substitutions are determined by those skilled in the art at the time such substitutions are desired. For example, amino acid substitutions are used to identify important residues of the molecule sequence, or to increase or decrease the affinity of the molecules described herein. The following eight groups contain amino acids that are conservative amino acid substitutions for one another: 1) Alanine (A). Glycine (G); 2) Aspartic acid (D), Glutamic acid (E); 3) Asparagine (N), Glutamine (Q); 4) Arginine (R), Lysine (K); 5) Isoleucine (1). Leucine (L), Methionine (M), Valine (V); 6) Phenylalanine (F), Tyrosine (Y), Tryptophan (W); 7) Serine (S), Threonine (T); and 8) Cysteine (C), Methionine (M).

In some embodiments, the antibody or antigen binding fragment thereof binds to human CD19. In some embodiments, the antibody or antigen binding fragment binding CD19 is a single-chain variable fragment (scFv). In embodiments involving a single polypeptide containing both a heavy chain variable region and a light chain variable region, both orientations of these variable regions are contemplated. In some embodiments, the heavy chain variable region is on the N-terminal side of the light chain variable region, which means the heavy chain variable region is closer to the N-terminus of the polypeptide. In other embodiments, the light chain variable region is on the N-terminal side of the heavy chain variable region, which means the light chain variable region is closer to the N-terminus of the polypeptide than the heavy chain variable region.

In some embodiments, the scFv binding proteins comprise a linker. In some embodiments, the linker is between the heavy chain variable region (VH) and the light chain variable region (VL) (or vice versa). In some embodiments, the linker comprises the amino acid sequence of GS, GGS, GGGS (SEQ ID NO:227), GGGGS (SEQ ID NO:147), GGGGGS (SEQ ID NO:145), any one of SEQ ID NOs:165-166 and 32-33, or combinations thereof. Substitutions to introduce new disulfide bonds are also within the scope of the disclosure, e.g., by making substitutions G44C in the VH FR 2 and G100C in the VL FR4.

In some embodiments, the anti-CD19 antibody or antigen binding fragment binds to human CD19 with an affinity constant (K_{D}) of between about 1 nM and about 900 nM. In some embodiments, the K_{D} to human CD19 is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the anti-CD19 antibody or antigen binding fragment binds to human CD19 with an affinity constant (K_{D}) of 500 nM, 400 nM, 300 nM, 200 nM, 100 nM, 90 nM, 80 nM, 70 nM, 60 nM, 50 nM, 40 nM, 30 nM, 20 nM, or 10 nM or lower. In some embodiments, the anti-CD19 antibody or antigen binding fragment binds to human CD19 and cynomolgus, M. mulatta (rhesus monkey), or M. nemestrina CD19 with comparable binding affinity (K_{D}).

In some embodiments, the anti-CD19 antibody or antigen binding fragment binds to cynomolgus, M. mulatta (rhesus monkey), or N. nemestrina CD19. In some embodiments, the anti-CD19 antibody or antigen binding binds to mouse, dog, pig. etc., CD19. In some embodiments, the K_{D} to cynomolgus or M. nemestrina CD19 is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM. about 360 nM to about 400 nM, about 410 nM to about 450 nM, and about 460 nM to about 500 nM. In same embodiments, the anti-CD19 antibody or antigen binding fragment binds to cynomolgus or M. nemestrina CD19 with an affinity constant (K_{D}) of 500 nM. 400 nM, 300 nM, 200 nM, 100 nM, 90 nM, 80 nM, 70 nM, 60 nM, 50 nM, 40 nM, 30 nM, 20 nM, or 10 nM or lower.

An antibody or antigen binding fragment thereof that specifically binds CD19 refers to an antibody or binding fragment that preferentially binds to CD19, respectively, over other antigen targets. As used herein, the term is interchangeable with an “anti-CD19” antibody or an “antibody that binds CD19.” In some embodiments, the antibody or binding fragment capable of binding to CD19 can do so with higher affinity for that antigen than others. In some embodiments, the antibody or binding fragment capable of binding CD19 can bind to that antigen with a K_{D} of at least about 10⁻¹, 10⁻², 10⁻³,10⁻⁴, 10⁻⁵, 10⁻⁶, 10⁻⁷, 10⁻⁸, 10⁻⁹, 10⁻¹⁰,10⁻¹¹, 10⁻¹² or greater (or any value in between), e.g., as measured by surface plasmon resonance or other methods known to those skilled in the art.

In some embodiments, the antibody or antigen binding fragment thereof is bispecific. In some embodiments, the bispecific antibody or antigen binding fragment comprises an antibody or antigen binding fragment thereof that specifically binds CD19 as herein disclosed and an antigen or antibody binding fragment thereof that specifically binds CD3, 4-1BB, IL-6, NKG2D, Fc-gamma-RIIIA (CD16), APRIL, CD38, TACI, Fc-gamma-RIIIA (CD16) and NKG2D, CD3 and serum albumin, CD47 and TACI, or CD3 and GPRC5D.

In some embodiments, the antibody or antigen binding fragment thereof is conjugated. In some embodiments, the antibody or antigen-binding fragment thereof is an antibody-drug conjugate, wherein the antibody or antigen binding fragment thereof that specifically binds CD19 as herein disclosed is conjugated to a therapeutic agent or diagnostic agent. In some embodiments, the antibody or antigen binding fragment thereof is conjugated to a tag for detection. In some embodiments, the antibody or antigen binding fragment thereof is conjugated to a conjugate that enhances antibody or antigen binding fragment thereof stability. In some embodiments, the antibody or antigen binding fragment thereof is conjugated to a cleavable linker, wherein the linker allows for another molecule to be conjugated to the antibody or antigen binding fragment thereof. In some embodiments, the antibody or antigen binding fragment thereof is conjugated to a nanoparticle.

Some embodiments of the disclosure are an isolated polynucleotide encoding any of the antibody heavy chain variable regions or the antibody light chain variable regions of the disclosure. Certain exemplary polynucleotides are disclosed herein, however, other polynucleotides which, given the degeneracy of the genetic code or codon preferences in a given expression system, encode the antibodies or antigen binding fragments thereof of the disclosure are also within the scope of the disclosure. The polynucleotide sequences encoding a VH or a VL or a fragment thereof of the to antibody or antigen binding fragments thereof of the disclosure are operably linked to one or more regulatory elements, such as a promoter and enhancer, that allow expression of the nucleotide sequence in the intended host cell. In some embodiments, the polynucleotide is a cDNA.

Some embodiments of the disclosure are a vector comprising the polynucleotide of the disclosure. In some embodiments, such vectors are plasmid vectors, viral vectors, vectors for baculovirus expression, transposon-based vectors, or any other vector suitable for introduction of the polynucleotide of the disclosure into a given organism or genetic background by any means. In some embodiments, the vector is polycistronic. For example, polynucleotides encoding light and heavy chain variable regions of the antibodies of the disclosure, optionally linked to constant regions, are inserted into expression vectors. The light and heavy chains are cloned in the same or different expression vectors. The DNA segments encoding immunoglobulin chains are operably linked to control sequences in the expression vector(s) that ensure the expression of immunoglobulin polypeptides. Such control sequences include signal sequences, promoters (e.g., naturally associated or heterologous promoters), enhancer elements, and transcription termination sequences, and are chosen to be compatible with the host cell chosen to express the antibody. In some embodiments, the polycistronic vector comprises one or more tolerogenic factor, safety switch, additional antibodies or antigen binding fragments thereof, or other regulatory elements as disclosed herein. Once the vector has been incorporated into the appropriate host, the host is maintained under conditions suitable for high level expression of the proteins encoded by the incorporated polynucleotides.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance, or neomycin resistance to permit detection of those cells transformed with the desired DNA sequences. Suitable vectors, promoter, and enhancer elements are known in the art; many are commercially available for generating subject recombinant constructs.

Some embodiments of the disclosure are a host cell comprising the vector of the disclosure. The term “host cell” refers to a cell into which a vector has been to introduced. It is understood that the term host cell is intended to refer not only to the particular subject cel but to the progeny of such a cell. Because certain modifications may occur in succeeding generations due to either mutation or environmental influences, such progeny may not be identical to the parent cell, but are still included within the scope of the term “host cell” as used herein. Such host cells include eukaryotic cells, prokaryotic cells, plant cells, or archaeal cells. Escherichia coli, bacilli, such as Bacillus subtilis, and other enterobacteriaceae, such as Salmonella. Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells include cells of mammalian, insect, avian, or other animal origins.

### CD19 Chimeric Antigen Receptor

In some embodiments the provided disclosure relates to chimeric receptors, such as a chimeric antigen receptor (CAR), that contain one or more domains that combine an antigen- or ligand-binding domain (e.g., antibody or antigen binding fragment thereof) that provides specificity for a desired antigen (e.g., tumor antigen) with intracellular signaling domains. In some embodiments, the intracellular signaling domain is a stimulating or an activating intracellular domain portion, such as a T cell stimulating or activating domain, providing a primary activation signal or a primary signal. In some embodiments, the intracellular signaling domain contains or additionally contains a costimulatory signaling domain to facilitate effector functions. In some embodiments, chimeric receptors when genetically engineered into immune cells can modulate T cell activity, and, in some embodiments, can modulate T cell differentiation or homeostasis, thereby resulting in genetically engineered cells with improved longevity, survival and/or persistence in vivo, such as for use in adoptive cell therapy methods.

In some embodiments, the chimeric antigen receptor includes an extracellular portion containing an antibody or antigen binding fragment thereof that comprises an antigen-binding domain. In some aspects, the chimeric antigen receptor includes an extracellular portion containing the antibody or antigen binding fragment thereof comprising an antigen-binding domain and an intracellular signaling domain. In some embodiments, the antibody or antigen binding fragment thereof includes an scFv.

In some embodiments, the antigen targeted by the antigen-binding domain is CD19. In some aspects, the antigen-binding domain of the recombinant receptor, e.g., CAR, binds, such as specifically binds or specifically recognizes, a CD19, such as a human CD19. In some embodiments, the antibody or antigen binding fragment thereof comprises a VH and a VL derived from an antibody or an antibody fragment specific to CD19 as disclosed herein. In some embodiments, the antibody or antigen binding fragment thereof is a human antibody, e.g., as described in U.S. Patent Publication No. US 2016/0152723.

In some embodiments, the CAR is a CD19 CAR (“CD19-CAR”). In some of these embodiments, a polycistronic vector comprises an expression cassette that contains a nucleotide sequence encoding a CD19 CAR or another CAR disclosed herein. CD19 is a immunoglobin (Ig) superfamily member expressed on cells of the B cell lineage. CD19 is a biomarker for B cell development. The expression of CD19 has been linked to a number of cancers, such as B cell lymphomas, acute lymphoblastic leukemia (ALL), and chronic lymphocytic leukemia (CLL). In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the CD19 specific CAR includes an antibody or antigen binding fragment thereof, a transmembrane domain, a co-stimulatory signaling domain, and a signaling domain. In some embodiments, the antibody or antigen binding fragment thereof is an anti-CD19 single-chain antibody fragment (scFv) or single-domain antibody fragment (sdAb). Table 11 provides several non-limiting exemplary sequences of full-length CD19 scFv and sdAb sequences. In some embodiments, the CD19 specific CAR includes an anti-CD19 single-chain antibody fragment (scFv) or single-domain antibody fragment (sdAb), a transmembrane domain such as one derived from human CD8α, a 4-1BB (CD137) co-stimulatory signaling domain, and a CD3ζ signaling domain. In some embodiments, the CAR is bispecific and specifically binds human CD19 and another tumor antigen selected from CD5, CD19, CD20, CD22, CD23. CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRa, IL-13Ra, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, G02, Goa. HMW-MAA, IL-11Ra, KDR, Lewis Y, L1-cell adhesion molecule, MADE-A1, Oncofetal antigen (h5T4), TAG-72, CD19/22, Syndecan 1, or BCMA. In some embodiments, the bispecific CAR includes an anti-CD19 scFv, a scFv that specifically binds one of CD5, CD19, CD20, CD22, CD23, CD30, CD33, CD38, C70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRa, IL-13Ra, Mesothelin, MUC1, MUC16. ROR1, C-Met, CD1133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-82, erb-B, FBP, Fetal acetylcholine e receptor, G02, Ga. HMW-MAA, IL-11Ra, KDR, Lewis Y, L1-cell adhesion molecule, MADE-A1, Oncofetal antigen (h5T4), TAG-72, CD19/22, Syndecan 1, or BCMA, a transmembrane domain, a co-stimulatory signaling domain, and a signaling domain. In some embodiments, the bispecific CAR includes an anti-CD19 scFv, a scFv that specifically binds one of CD5, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRa, IL-13Ro, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, GD2, Ga. HMW-MAA, IL-11Ra, KDR, Lewis Y, L1-cell adhesion molecule, MADE-A1, Oncofetal antigen (h5T4), TAG-72, CD19/22, Syndecan 1, or BCMA, a transmembrane domain such as one derived from human CD8α, a 4-1BB (CD137) co-stimulatory signaling domain, and a CD34 signaling domain.

In some embodiments, the signal peptide of the CD19 CAR comprises a CD8α signal peptide. In some embodiments, the CD8a signal peptide comprises or consists of an amino acid sequence set forth in SEQ ID NO: 28 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 28. In some embodiments, the signal peptide comprises an IgK signal peptide. In some embodiments, the IgK signal peptide comprises or consists of an amino acid sequence set forth in SEQ ID NO: 29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:29. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO: 30 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 30. In some embodiments, the signal peptide comprises a Immunoglobulin heavy chain signal peptide. In some embodiments, the Immunoglobulin heavy chain signal peptide comprises or consists of an amino acid sequence set forth in SEQ ID NO: 31 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 31. Table 12 provides several non-limiting examples of sequences of exemplary signal peptides.

In some embodiments, the extracellular binding domain of the CD19 CAR is specific to CD19, for example, human CD19. The extracellular binding domain of the CD19 CAR is codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the CD19 CAR is derived from an antibody specific to CD19, including, for example, any one of the antibodies or antigen binding fragments thereof herein disclosed, belantamab, erlanatamab, teclistamab, LCAR-B38M, and cittacabtagene. In any of these embodiments, the extracellular binding domain of the CD19 CAR can comprise or consist of the VH. the VL, and/or one or more CDRs of any of the antibodies or antigen binding fragments thereof disclosed herein.

In some embodiments, the extracellular binding domain of the CD19 CAR comprises an scFv. The scFv may comprise the heavy chain variable region (VH) and the light chain variable region (V_{L}) connected by a (G4S)3 linker or by a Whitlow linker, the amino acid sequences of which set forth in SEQ ID NO: 32 and 33, respectively, set forth in Table 13. In some embodiments, the CD19-specific extracellular binding domain comprises or consists of an amino acid sequence set forth in SEQ ID NO: 25, 26, or 27, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:25-27 or 228-230, set forth in Table 11. In some embodiments, the CD19-specific extracellular binding domain may comprise one or more heavy chain CDRs having amino acid sequences set forth in Table 3 and one or more light chain CDRs having amino acid sequences set forth in Table 4. In some embodiments, the CD19-specific extracellular binding domain may comprise a heavy chain having amino acid sequences set forth in Table 5. In some embodiments, the CD19-specific extracellular binding domain may comprise a light chain having amino acid sequences set forth in Table 6. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In any of these embodiments, the CD19-specific scFv may comprise one or more heavy chains (VH) comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In any of these embodiments, the CD19-specific scFv may comprise one or more light chains (VL) comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the extracellular binding domain of the CD19 CAR comprises single variable fragments of a heavy chain (VH) that can bind to an epitopes of CD19.

In some embodiments, the extracellular binding domain of the CD19 CAR comprises a single domain antibody (sdAb). In any of these embodiments, the CD19-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the hinge domain of the CD19 CAR comprises a CD8a hinge domain, for example, a human CD8a hinge domain. In some embodiments, the CD8a hinge domain comprises or consists of an amino acid sequence set forth in SEQ ID NO: 34 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 34. In some embodiments, the hinge domain comprises a CD28 hinge domain, for example, a human CD28 hinge domain. In some embodiments, the CD28 hinge domain comprises or consists of an amino acid sequence set forth in SEQ ID NO: 35 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 35. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO: 37 or SEQ ID NO: 38, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 37 or SEQ ID NO: 38. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO: 39 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 39. Non-limiting exemplary sequences of hinge domains are set forth in Table 14.

In some embodiments, the transmembrane domain comprises one selected from a group that includes a transmembrane region of TCRα, TCRβ, TCRζ, CD3ε, CD3γ, CD36, CD3ζ, CD4, CDS, CD8α, CD8β, CD9, CD18, CD28, CD45, CD22, CD33, CD34, CD37, CD40, CD40UCD154, CD45, CD64, CD8β, CD86, OX40/CD134, 4-1BB/CD137, CD154, FcεRI γ, VEGFR2, FAS, FGFR21, and functional variant thereof.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO: 40 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 40. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 41 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 41. In some embodiments, the CD28 transmembrane domain comprises or consists of an amino acid sequence set forth in SEQ ID NO: 42 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 42. Non-limiting exemplary sequences of transmembrane domains are set forth in Table 15.

In some embodiments, the signaling domain(s) of the CAR comprises a costimulatory domain(s). For instance, a signaling domain can contain a costimulatory domain. Or, a signaling domain can contain one or more costimulatory domains. In some embodiments, the signaling domain comprises a costimulatory domain. In other embodiments, the signaling domains comprise costimulatory domains. In some embodiments, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR-T cell proliferation, and/or CAR-T cell persistence during T cell activation. In some embodiments, the costimulatory domains enhance cytokine production, CAR-T cell proliferation, and/or CAR-T cell persistence during T cell activation.

In some embodiments, the intracellular costimulatory domain of the CD19 CAR comprises a 4-1BB costimulatory domain, for example, a human 4-1BB costimulatory domain. In some embodiments, the 4-1BB costimulatory domain comprises or consists of an amino acid sequence set forth in SEQ ID NO: 43 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 43. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 44 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 44. In some embodiments, the CD34 signaling domain of SEQ ID NO:46 may have a mutation, e.g., a glutamine (0) to lysine (K) mutation, at amino acid position 14 (see SEQ ID NO:61). Non-limiting exemplary sequences of intracellular costimulatory and/or signaling domains are set forth in Table 16.

In some embodiments, the intracellular signaling domain of the CD19 CAR comprises a CD3 zeta (ζ) signaling domain, for example, a human CD3ζ signaling domain. In some embodiments, the CD3ζ signaling domain comprises or consists of an amino acid sequence set forth in SEQ ID NO: 46 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 46.

In some embodiments, the CD19 CAR has a corresponding amino acid sequence set forth in SEQ ID NOs: 232, 234, 236, 238, 240, or 242 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 232, 234, 236, 238, 240, or 242. Non-limiting exemplary amino acid sequences of CD19 CARs are set forth in Table 35.

In some embodiments, the CD19 CAR is encoded by a nucleotide sequence set forth in SEQ ID NOs: 233, 235, 237. 239, 241, or 243 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 233, 235, 237, 239, 241, or 243. Non-limiting exemplary nucleotide sequences of CD19 CARs are set forth in Table 35.

In some embodiments, the polycistronic vector comprises an expression cassette that contains a nucleotide sequence encoding a CD19 CAR, including, for example, a CD19 CAR comprising any of the CD19-specific extracellular binding domains as described, the CD8α hinge domain of SEQ ID NO: 34, the CD8α transmembrane domain of SEQ ID NO: 40, the 4-1BB costimulatory domain of SEQ ID NO: 43, the CD3ζ signaling domain of SEQ ID NO: 46, and/or variants (i.e., having a sequence that is at least 80% identical. for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising any of the CD19-specific extracellular binding domains as described, the CD8α hinge domain of SEQ ID NO: 34, the CD8α transmembrane domain of SEQ ID NO: 40, the CD28 costimulatory domain of SEQ ID NO: 44, the CD3; signaling domain of SEQ ID NO: 46, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide as described.

In some embodiments, the antibody portion of the recombinant receptor, e.g., CAR, further includes spacer between the transmembrane domain and extracellular antigen binding domain. In some embodiments, the spacer includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgGI. In some aspects, the portion of the constant region serves as a spacer region between the antigen-recognition component, e.g., scFv, and transmembrane domain. The spacer is of a length that provides for increased responsiveness of the cell following antigen binding, as compared to in the absence of the spacer. Exemplary spacers include, but are not limited to, those described in Hudecek et al. (2013) Clin. Cancer Res., 19:3153, WO2014031687. U.S. Pat. No. 8,822,647 or published app. No. US 2014/0271635. In some embodiments, the constant region or portion is of a human IgG, such as IgG4 or IgGI.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g., extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g., scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD8β. CD86, CD 134, CD 137, CD 154. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain are linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act In a stimulatory manner may contain signaling motifs which are known as immunoreceptor tyrosine-based activation motifs or IT AMs. Examples of IT AM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the intracellular component is or includes a CD3-zeta intracellular signaling domain. In some embodiments, the intracellular component is or includes a signaling domain from Fc receptor gamma chain. In some embodiments, the receptor, e.g., CAR, includes the intracellular signaling domain and further includes a portion, such as a transmembrane domain and/or hinge portion, of one or more additional molecules such as CD8, CD4, CD25, or CD 16. For example, in some aspects, the CAR or other chimeric receptor is a chimeric molecule of CD3-zeta (CD3-z) or Fc receptor g and a portion of one of CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, 0X40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-188.

In some embodiments the Intracellular signaling domain includes intracellular components of a 4-1BB signaling domain and a CD3-zeta signaling domain. In some embodiments, the intracellular signaling domain includes intracellular components of a CD28 signaling domain and a CD3zeta signaling domain.

In some embodiments, the CAR comprises an extracellular antigen binding domain (e.g., antibody or antibody fragment, such as an scFv) that binds to an antigen (e.g., tumor antigen), a spacer (e.g., containing a hinge domain, such as any as described herein), a transmembrane domain (e.g., any as described herein), and an intracellular signaling domain (e.g., any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain). Non-limiting examples of exemplary components of a CAR are described In Table 17. In provided aspects, the sequences of each component in a CAR include any combination listed in Table 17.

In some embodiments, the antigen receptor further includes a marker and/or cells expressing the CAR or other antigen receptor further includes a surrogate marker, such as a cell surface marker, which is used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, is any as disclosed in published patent application No. WO2014031687. For example, the marker is a truncated EGFR (tEGFR) that is, optionally, linked to a linker sequence, such as a T2A cleavable linker sequence.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker is a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, CARs are referred to as first, second, third generation, and/or fourth generation CARs. In some embodiments, the CAR disclosed herein is selected from a group including: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

As described herein, a fourth generation CAR can contain an antigen binding domain, a transmembrane domain, three or four signaling domains, and a domain which upon successful signaling of the CAR induces expression of a cytokine gene. In some instances, the cytokine gene is an endogenous or exogenous cytokine gene of the hypoimmunogenic cells. In some embodiments, the cytokine gene encodes a pro-inflammatory cytokine. In some embodiments, the pro-inflammatory cytokine is selected from a group that includes IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR contains an antibody, e.g., an antibody fragment, as disclosed herein, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, as disclosed herein, a transmembrane domain that is or contains a transmembrane portion of CD28 or a functional variant thereof, and an intracellular signaling domain containing a signaling portion of a 4-IBB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g., an IgG4 hinge, such as a hinge-only spacer.

In some aspects, the spacer contains only a hinge region of an IgG, such as only a hinge of IgG4 or IgG. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, the CAR includes an antibody such as an antibody fragment, including scFvs and sdAbs as disclosed herein, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv or sdAb as disclosed herein, a spacer such as any of the Ig-hinge containing spacers, a CD28-derived transmembrane domain, a 4-IBB-derived intracellular signaling domain, and a CD3 zeta-derived signaling domain.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

Some embodiments of the disclosure are an isolated polynucleotide encoding any of the CARs or CAR components of the disclosure. Certain exemplary polynucleotides are disclosed herein, however, other polynucleotides which, given the degeneracy of the genetic code or codon preferences in a given expression system, encode the antibodies or antigen binding fragments thereof of the disclosure are also within the scope of the disclosure. The polynucleotide sequences encoding the CARs or CAR components thereof of the disclosure are operably linked to one or more regulatory elements, such as a promoter and enhancer, that allow expression of the nucleotide sequence in the intended host cell. The polynucleotide is a cDNA.

Some embodiments of the disclosure are a vector comprising the polynucleotide of the disclosure. In some embodiments, such vectors are plasmid vectors, viral vectors, vectors for baculovirus expression, transposon-based vectors, or any other vector suitable for introduction of the polynucleotide of the disclosure into a given organism or genetic background by any means. For example, polynucleotides encoding light and heavy chain variable regions of the antibodies of the disclosure, optionally linked to constant regions, are inserted into expression vectors. The light and heavy chains are cloned in the same or different expression vectors. In some embodiments, the DNA segments encoding immunoglobulin chains are operably linked to control sequences in the expression vector(s) that ensure the expression of immunoglobulin polypeptides. Such control sequences include signal sequences, promoters (e.g., naturally associated or heterologous promoters), enhancer elements, and transcription termination sequences, and are chosen to be compatible with the host cell chosen to express the antibody. Once the vector has been incorporated into the appropriate host, the host is maintained under conditions suitable for high level expression of the proteins encoded by the incorporated polynucleotides.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance, or neomycin resistance to permit detection of those cells transformed with the desired DNA sequences. Suitable vectors, promoter, and enhancer elements are known in the art; many are commercially available for generating subject recombinant constructs.

Some embodiments of the disclosure are a method of producing a CAR, comprising delivering a polynucleotide encoding a CAR as herein described, or a vector comprising a polynucleotide encoding a CAR as herein described to a host cell. In some embodiments, the method of delivery of the polynucleotide or the vector is any method for delivery of nucleic acids known to those skilled in the art, and include, but are not limited to, transfection, transduction, electroporation, and transformation.

Some embodiments of the disclosure are a host cell comprising the vector of the disclosure. The term “host cell” refers to a cell into which a vector has been introduced. It is understood that the term host cell is intended to refer not only to the particular subject cell but to the progeny of such a cell. Because certain modifications may occur in succeeding generations due to either mutation or environmental influences, such progeny may not be identical to the parent cell, but are still included within the scope of the term “host cell” as used herein. Such host cells include eukaryotic cells, prokaryotic cells, plant cells, or archaeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells are of mammalian. insect, avian, or other animal origins.

### Vector for Delivering a CAR

Also provided herein are targeted lipid particles (e.g., vectors) that comprise a targeting antibody or antigen binding fragment thereof for delivery of the targeted lipid particle to a target cell and an exogenous agent. In some embodiments, the targeted lipid particle comprises a henipavirus F protein molecule or a biologically active portion thereof. In some embodiments the targeted lipid particle comprises a henipavirus G protein molecule or a biologically active portion thereof. In some embodiments, the targeted lipid particle comprises a henipavirus F protein molecule or biologically active portion thereof and a henipavirus G protein molecule or biologically active portion thereof.

In some embodiments, the targeting antibody or antigen binding fragment thereof is attached on a membrane-bound protein of the targeted lipid particle. In other embodiments, the targeting antibody or antigen binding fragment thereof is attached to a fusogen on the outer surface of the targeted lipid particle. In some embodiments the targeting antibody or antigen binding fragment thereof is attached to the henipavirus G protein or a biologically active portion thereof. In some embodiments, the targeting antibody or antigen binding fragment thereof is attached to the henipavirus G protein or a biologically active portion thereof, for example, as described in U.S. Patent Publication 2022/0333134A1, which is hereby incorporated by reference in its entirety.

In some embodiments, the target cell is an immune cell. In some embodiments, the immune cell is a NK cell, a T cell, a macrophage, or a monocyte. In some embodiments, the immune cell is a T cell. In some embodiments, the T cell is a CD3+ T cell, a CD4+ T cell, a CDS+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T cel, an effector memory T cell, an effector memory T cell expressing CD45RA (TEMRA cell), a tissue-resident memory (Trm) cell, a virtual memory T cell, an innate memory T cell, a memory stem cell (Tse), or a γδ T cell. In some embodiments, the T cell is a cytotoxic T cell, a helper T cell, a memory T cell, a regulatory T cell, or a tumor infiltrating lymphocyte. In some embodiments, the T cell is a CD4+ T cell. In other embodiments, the T cell is a CD8+ T cell.

### A. Lipid Bilayer

In some embodiments, the targeted lipid particle includes a naturally derived bilayer of amphipathic lipids that encloses a lumen or cavity. In some embodiments, the targeted lipid particle comprises a lipid bilayer as the outermost surface. In some embodiments, the lipid bilayer encloses a lumen. In some embodiments, the lumen is aqueous. In some embodiments, the lumen is in contact with the hydrophilic head groups on the interior of the lipid bilayer. In some embodiments, the lumen is a cytosol. In some embodiments, the cytosol contains cellular components present in a source cell. In some embodiments, the cytosol does not contain cellular components present in a source cell. In some embodiments, the lumen is a cavity. In some embodiments, the cavity contains an aqueous environment. In some embodiments, the cavity does not contain an aqueous environment.

In some aspects, the lipid bilayer is derived from a source cell during a process to produce a lipid-containing particle. In some embodiments, the lipid bilayer includes membrane components of the cell from which the lipid bilayer is produced, e.g., phospholipids, membrane proteins, etc. In some embodiments, the lipid bilayer includes a cytosol that includes components found in the cell from which the lipid bilayer is produced, e.g., solutes, proteins, nucleic acids, etc., but not all of the components of a cell, e.g., it lacks a nucleus. In some embodiments, the lipid bilayer is considered to be exosome-like. The lipid particle may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a source cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In other aspects, the lipid bilayer includes synthetic lipid complex. In some embodiments, the synthetic lipid complex is a liposome. In some embodiments, the lipid particle is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, a targeted envelope protein and fusogen, such as any described above including any that are exogenous or overexpressed relative to the source cell, is disposed in the lipid bilayer.

In some embodiments, the targeted lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

In some embodiments, the bilayer is comprised of one or more lipids of the same or different type. In some embodiments, the source cell comprises a cell selected from CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

### B. Targeting Antibody

In some aspects, the targeted lipid particles (e.g., vectors) comprise a targeting antibody or antigen binding fragment thereof for delivery of the targeted lipid particle to a target cell.

In some embodiments, the targeting antibody or antigen binding fragment thereof is attached on a membrane-bound protein of the targeted lipid particle. In other embodiments, the targeting antibody or antigen binding fragment thereof is attached to a fusogen on the outer surface of the targeted lipid particle. In some embodiments the targeting antibody or antigen binding fragment thereof is attached to a henipavirus G protein or a biologically active portion thereof. In some embodiments, the C-terminus of the targeting antibody or antigen binding fragment thereof is attached to the C-terminus of a G protein or biologically active portion thereof. In some embodiments, the N-terminus end of the targeting antibody or antigen binding fragment thereof is exposed on the exterior surface of the lipid bilayer. In some embodiments, the N-terminus end of the targeting antibody or antigen binding fragment thereof binds to a cell surface molecule of a target cell. In some embodiments, the targeting antibody or antigen binding fragment thereof specifically binds to a cell surface molecule present on a target cell. In some embodiments, the cell surface molecule is a protein, glycan, lipid, or low molecular weight molecule.

In some embodiments, the cell surface molecule of a target cell is an antigen or portion thereof. In some embodiments, the targeting antibody or antigen binding fragment thereof is an antibody having a single monomeric domain antigen binding/recognition domain that is able to bind selectively to a specific antigen. In some embodiments, the single domain antibody binds an antigen present on a target cell. In some embodiments, the cell surface molecule is CD4 or CD8.

Exemplary cells include immune effector cells, peripheral blood mononuclear cells (PBMC) such as lymphocytes (T cells, B cells, natural killer cells) and monocytes, granulocytes (neutrophils, basophils, eosinophils), macrophages, dendritic cells, cytotoxic T lymphocytes, polymorphonuclear cells (also known as PMN, PML, or PMNL), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, allogenic cells, resident cardiac cells, induced pluripotent stem cells (iPS), adipose-derived or phenotypic modified stem or progenitor cells, C133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes.

In some embodiments, the target cell is a cell of a target tissue. In some embodiments, the target tissue is liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g., hepatocyte), or a cardiac cell (e.g., cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g., a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+B cell, a CD19+B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacytoid dendritic cell, a CD11c+ cell, a CD11b+ cell, a splenocyte, a B cell, a hepatocyte, an endothelial cell, or a non-cancerous cell.

### I. CD4 Antibody

In some embodiments, the targeting antibody or antigen binding fragment thereof that specifically target and bind CD4 for delivery of the targeted lipid particle to a cell expressing CD4. In some embodiments, the antibodies or antigen binding fragments thereof may cross-react with cynomolgus (or “cyno”) or M. nemestrina CD4. In some embodiments, the antibodies or antigen binding fragments thereof are single-chain variable fragments (scFvs) composed of the antigen-binding domains derived from the heavy (VH) and the light (VL) chains of the IgG molecule and connected via a linker domain. In some embodiments, the antibodies or antigen binding fragments thereof are VHHs that correspond to the VH of the IgG molecule. The present disclosure also provides polynucleotides encoding the antibodies and fragments thereof, vectors, and host cells, and methods of using the antibodies or antigen binding fragments thereof. In some embodiments, e.g., the antibodies or antigen binding fragments thereof are fused to henipavirus glycoprotein G for targeted binding and transduction to cells.

Sequences for exemplary antibodies and antigen binding fragments of the disclosure using the Kabat numbering scheme are shown in Tables 18-19 below. Sequences for exemplary HCDRs of the disclosure are shown in Table 18. Sequences for exemplary LCDRs of the disclosure are shown in Table 19. Additional suitable sequences of antibodies or antigen binding fragments thereof that specifically bind CD4 are disclosed, for example, in U.S. Provisional Application No. 63/326,269 and U.S. Provisional Application No. 63/341,681, which are hereby incorporated by reference in their entirety.

The sequences for the disclosed VH and VL domains are provided in Tables 20-21.

In some embodiments, an antibody or antigen binding fragment thereof capable of binding CD4 is disclosed, comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3). In some embodiments, the HCDR1. HCDR2, and HCDR3 comprise amino acid sequences of any one of the SEQ ID NOs recited in Table 18 and the LCDR1. LCDR2, and LCDR3 comprise amino acid sequences of any one of the SEQ ID NO recited in Table 19. In some embodiments, the heavy chain variable region (VH) comprises an amino acid sequence of any one of SEQ ID NOs: 71-74 (Table 20) and the light chain variable region (VL) comprises an amino acid sequence of any one of SEQ ID NOs: 75-77 (Table 21).

In some embodiments, the antibody or antigen binding fragment thereof comprises a VH having an amino acid sequence with at least 80%, 85%, 90%, 95%, 96%, 97%, 98%, 99%, or 100% identity to a sequence selected from SEQ ID NOs: 71-74.

In some embodiments, the antibody or antigen binding fragment thereof comprises a VL having an amino acid sequence with at least 80%, 85%, 90%, 95%, 96%, 97%, 98%, 99%, or 100% identity to a sequence selected from SEQ ID NOs: 75-77.

In some embodiments, the antibody or antigen binding fragment comprises a VH having an amino acid sequence with at least 80%, 85%, 90%, 95%, 96%, 97%, 98%, 99%, or 100% identity to a sequence selected from SEQ ID NOs: 71-74 and a VL having an amino acid sequence with at least 80%, 85%, 90%, 95%, 96%, 97%, 98%, 99%, or 100% identity to a sequence selected from SEQ ID NOs: 75-77.

In some embodiments, the antibody or antigen binding fragment thereof comprises the HCDR1, HCDR2, HCDR3, LCDR1, LCDR2, and LCDR3 of SEQ ID NOs: 50, 54, 58, 62, 65, and 68, respectively, In some embodiments, the antibody or antigen binding fragment thereof comprises the HCDR1, HCDR2, HCDR3, LCDR1, LCDR2, and LCDR3 of SEQ ID NOs: 51, 55, 59, 63, 66, and 69, respectively.

In some embodiments, the antibody or antigen binding fragment thereof comprises the HCDR1, HCDR2, HCDR3, LCDR1, LCDR2, and LCDR3 of SEQ ID NOs: 52, 56, 60, 64, 67, and 70, respectively.

In some embodiments, the antibody or antigen binding fragment thereof comprises the HCDR1, HCDR2, and HCDR3 of SEQ ID NOs: 53, 57, and 61, respectively.

In some embodiments, the single domain antibody is human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In various embodiments, any of the antibodies or antigen binding fragments described herein can comprise a heavy chain constant region and a light chain constant region. In some embodiments, the heavy chain constant region is an IgG, IgM, IgA, IgD, or IgE isotype, or a derivative or fragment thereof that retains at least one effector function of the intact heavy chain. In some embodiments, the heavy chain constant region is a human IgG isotype. In some embodiments, the heavy chain constant region is a human IgG1 or human IgG4 isotype. In some embodiments, the heavy chain constant region is a human IgG1 isotype. In some embodiments, the light chain constant region is a human kappa light chain or lambda light chain or a derivative or fragment thereof that retains at least one effector function of the intact light chain. In some embodiments, the light chain constant region is a human kappa light chain.

In various embodiments, any of the disclosed antibodies or antigen binding fragments are a rodent antibody or antigen binding fragment thereof, a chimeric antibody or an antigen binding fragment thereof, a CDR-grafted antibody or an antigen binding fragment thereof, or a humanized antibody or an antigen binding fragment thereof. In some embodiments, any of the disclosed antibodies or antigen binding fragments comprises human or human-derived heavy and light chain variable regions, including human frameworks or human frameworks with one or more backmutations. In various embodiments, any of the disclosed antibodies or antigen binding fragments are a Fab, Fab′, F(ab′)2, Fd, scFv, (scFv)2, scFv-Fc, VHH, or Fv fragment.

Antibodies whose heavy chain CDR, light chain CDR, VH, or VL amino acid sequences differ insubstantially from those shown in Tables 18-21 are encompassed within the scope of the disclosure. Typically, this involves one or more conservative amino acid substitutions with an amino acid having similar charge. hydrophobic, or stereo chemical characteristics in the antigen-binding site or in the framework without adversely altering the properties of the antibody. Conservative substitutions may also be made to improve antibody properties, for example stability or affinity. 1, 2, 3, 4, 5, 6, 7, 8, 9, 10, 11, 12, 13, 14, or 15 amino acid substitutions are made to the VH or VL sequence. For example, a “conservative amino acid substitution” may involve a substitution of a native amino acid residue with a nonnative residue such that there is little or no effect on the polarity or charge of the amino acid residue at that position. Desired amino acid substitutions are determined by those skilled in the art at the time such substitutions are desired. For example, amino acid substitutions are used to identify important residues of the molecule sequence, or to increase or decrease the affinity of the molecules described herein.

The following eight groups contain amino acids that are conservative amino acid substitutions for one another: 1) Alanine (A), Glycine (G); 2) Aspartic acid (D), Glutamic acid (E); 3) Asparagine (N), Glutamine (Q); 4) Arginine (R), Lysine (K): 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

In some embodiments, the antibody or antigen binding fragment thereof binds to human CD4. In some embodiments, the antibody or antigen binding fragment binding CD4 is a single-chain variable fragment. In embodiments involving a single polypeptide containing both a heavy chain variable region and a light chain variable region, both orientations of these variable regions are contemplated. In some embodiments, the heavy chain variable region is on the N-terminal side of the light chain variable region, which means the heavy chain variable region is closer to the N-terminus of the polypeptide. In other embodiments, the light chain variable region is on the N-terminal side of the heavy chain variable region, which means the light chain variable region is closer to the N-terminus of the polypeptide than the heavy chain variable region.

In some embodiments, the scFv binding proteins comprise a linker. In some embodiments, the linker is between the heavy chain variable region (VH) and the light chain variable region (VL) (or vice versa). In some embodiments, the linker comprises the amino acid sequence of GS, GGS, GGGS (SEQ ID NO:227), GGGGS (SEQ ID NO:147), GGGGGS (SEQ ID NO:145), any one of SEQ ID NOs:165-166 and 32-33, or combinations thereof. Substitutions to introduce new disulfide bonds are also within the scope of the disclosure, e.g., by making substitutions G44C in the VH FR 2 and G100C in the VL FR4.

In some embodiments, the anti-CD4 antibody or antigen binding fragment binds to human CD4 with an affinity constant (K_{D}) of between about 1 nM and about 900 nM. In some embodiments, the K_{D} to human CD4 is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the anti-CD4 antibody or antigen binding fragment binds to human CD4 with an affinity constant (K_{D}) of 500 nM, 400 nM, 300 nM, 200 nM, 100 nM, 50 nM, 20 nM, or 10 nM or lower. In some embodiments, the anti-CD4 antibody or antigen binding fragment binds to human CD4 and cynomolgus, M. mulatta (rhesus monkey), or M. nemestrina CD4 with comparable binding affinity (K_{D}).

In some embodiments, the anti-CD4 antibody or antigen binding fragment binds to cynomolgus, M. mulatta (rhesus monkey), or N. nemestrina CD4. In some embodiments, the anti-CD4 antibody or antigen binding binds to mouse, dog, pig, etc., CD4. In some embodiments, the K_{D} to cynomolgus or M. nemestrina CD4 is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the anti-CD4 antibody or antigen binding fragment binds to cynomolgus or M. nemestrina CD4 with an affinity constant (Kn) of 500 nM, 400 nM, 300 nM, 200 nM, 100 nM, 50 nM, 20 nM, or 10 nM or lower.

An antibody or antigen binding fragment thereof that specifically binds CD4 refers to an antibody or binding fragment that preferentially binds to CD4 over other antigen targets. As used herein, the term is interchangeable with an “anti-CD4” antibody or an “antibody that binds CD4.” In some embodiments, the antibody or binding fragment capable of binding to CD4 can do so with higher affinity for that antigen than others. In some embodiments, the antibody or binding fragment capable of binding CD4 can bind to that antigen with a K_{D} of at least about 10⁻¹, 10⁻², 10⁻³, 10⁻⁴, 10⁻⁵, 10⁻⁶, 10⁻⁷, 10⁻⁸, 10⁻⁹, 10⁻¹⁰,10⁻¹¹, 10⁻¹² or greater (or any value in between), e.g., as measured by surface plasmon resonance or other methods known to those skilled in the art.

ii. CD8 Antibody

In some embodiments, the targeting antibody or antigen binding fragment thereof that specifically target and bind CD8α or CD8β for delivery of the targeted lipid particle to a cell expressing CD8. In some embodiments, the antibodies or antigen binding fragments thereof may cross-react with cynomolgus (or “cyno”) or M. nemestrina CD8. In some embodiments, the antibodies or antigen binding fragments thereof are single-chain variable fragments (scFvs) composed of the antigen-binding domains derived from the heavy (VH) and the light (VL) chains of the 1gG molecule and connected via a linker domain. In some embodiments, the antibodies or antigen binding fragments thereof are VHHs that correspond to the VH of the IgG molecule. The present disclosure also provides polynucleotides encoding the antibodies and fragments thereof, vectors, and host cells, and methods of using the antibodies or antigen binding fragments thereof. In some embodiments, e.g., the antibodies or antigen binding fragments thereof are fused to henipavirus glycoprotein G for targeted binding and transduction to cells.

Sequences for exemplary antibodies and antigen binding fragments of the disclosure using the Kabat numbering scheme are shown in Tables 22-23 below. Sequences for exemplary HCDRs of the disclosure are shown in Table 22. Sequences for exemplary LCDRs of the disclosure are shown in Table 23. Additional suitable sequences of antibodies or antigen binding fragments thereof that specifically bind CD8 are disclosed, for example, in PCT Application Publication No. WO2022/216915, which is hereby incorporated by reference in its entirety.

The sequences for the disclosed VH and VL domains are provided in Tables 24-26.

In some embodiments, an antibody or antigen binding fragment thereof capable of binding CD8α or CD8p is disclosed, comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3). In some embodiments, the HCDR1, HCDR2, and HCDR3 comprise amino acid sequences of any one of the SEQ ID NOs recited in Table 22, and the LCDR1, LCDR2, and LCDR3 comprise amino acid sequences of any one of the SEQ ID NOs recited in Table 23. In some embodiments, the heavy chain variable region (VH) comprises an amino acid sequence of any one of SEQ ID NOs: 102-105 (Table 24) and the light chain variable region (VL) comprises an amino acid sequence of any one of SEQ ID NOs: 106-109 (Table 26).

In some embodiments, the antibody or antigen binding fragment thereof comprises a VH having an amino acid sequence with at least 80%, 85%, 90%, 95%, 96%, 97%, 98%, 99%, or 100% identity to a sequence selected from SEQ ID NOs: 102-105.

In some embodiments, the antibody or antigen binding fragment thereof comprises a VL having an amino acid sequence with at least 80%, 85%, 90%, 95%, 98%, 97%, 98%, 99%, or 100% identity to a sequence selected from SEQ ID NOs: 106-109.

In some embodiments, the antibody or antigen binding fragment comprises a VH having an amino acid sequence with at least 80%, 85%, 90%, 95%, 96%, 97%, 98%, 99%, or 100% identity to a sequence selected from SEQ ID NOs: 102-105 and a VL having an amino acid sequence with at least 80%, 85%, 90%, 95%, 96%, 97%, 98%, 99%, or 100% identity to a sequence selected from SEQ ID NOs: 106-109.

In some embodiments, the antibody or antigen binding fragment thereof comprises the HCDR1, HCDR2, HCDR3, LCDR1, LCDR2, and LCDR3 of SEQ ID NOs: 78, 82, 88, 90, 94, and 98, respectively.

In some embodiments, the antibody or antigen binding fragment thereof comprises the HCDR1, HCDR2, HCDR3, LCDR1, LCDR2, and LCDR3 of SEQ ID NOs: 79, 83, 87, 91, 95, and 99, respectively.

In some embodiments, the antibody or antigen binding fragment thereof comprises the HCDR1, HCDR2, HCDR3, LCDR1, LCDR2. and LCDR3 of SEQ ID NOs: 80, 84, 88, 92, 96, and 100, respectively.

In some embodiments, the antibody or antigen binding fragment thereof comprises the HCDR1, HCDR2, HCDR3, LCDR1, LCDR2, and LCDR3 of SEQ ID NOs: 81, 85, 89, 93, 97, and 101, respectively.

In some embodiments, the single domain antibody is human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In various embodiments, any of the antibodies or antigen binding fragments described herein can comprise a heavy chain constant region and a light chain constant region. In some embodiments, the heavy chain constant region is an IgG, IgM, IgA, IgD, or IgE isotype, or a derivative or fragment thereof that retains at least one effector function of the intact heavy chain. In some embodiments, the heavy chain constant region is a human IgG isotype. In some embodiments, the heavy chain constant region is a human IgG1 or human IgG4 isotype. In some embodiments, the heavy chain constant region is a human IgG1 isotype. In some embodiments, the light chain constant region is a human kappa light chain or lambda light chain or a derivative or fragment thereof that retains at least one effector function of the intact light chain. In some embodiments, the light chain constant region is a human kappa light chain.

In various embodiments, any of the disclosed antibodies or antigen binding fragments are a rodent antibody or antigen binding fragment thereof, a chimeric antibody or an antigen binding fragment thereof, a CDR-grafted antibody or an antigen binding fragment thereof, or a humanized antibody or an antigen binding fragment thereof. In some embodiments, any of the disclosed antibodies or antigen binding fragments comprises human or human-derived heavy and light chain variable regions, including human frameworks or human frameworks with one or more backmutations. In various embodiments, any of the disclosed antibodies or antigen binding fragments are a Fab, Fab′, F(ab′)2, Fd, scFv, (scFv)2, scFv-Fc, VHH, or Fv fragment.

Antibodies whose heavy chain CDR, light chain CDR. VH, or VL amino acid sequences differ insubstantially from those shown in Tables 22-25 are encompassed within the scope of the disclosure. Typically, this involves one or more conservative amino acid substitutions with an amino acid having similar charge, hydrophobic, or stereo chemical characteristics in the antigen-binding site or in the framework without adversely altering the properties of the antibody. Conservative substitutions may also be made to improve antibody properties, for example stability or affinity. 1, 2, 3, 4, 5, 6, 7, 8, 9, 10, 11, 12, 13, 14, or 15 amino acid substitutions are made to the VH or VL sequence. For example, a “conservative amino acid substitution” may involve a substitution of a native amino acid residue with a nonnative residue such that there is little or no effect on the polarity or charge of the amino acid residue at that position. Desired amino acid substitutions are determined by those skilled in the art at the time such substitutions are desired. For example, amino acid substitutions are used to identify important residues of the molecule sequence, or to increase or decrease the affinity of the molecules described herein. The following eight groups contain amino acids that are conservative amino acid substitutions for one another: 1) Alanine (A), Glycine (G); 2) Aspartic acid (D), Glutamic acid (E); 3) Asparagine (N), Glutamine (Q); 4) Arginine (R), Lysine (K): 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

In some embodiments, the antibody or antigen binding fragment thereof binds to human CD8α or CD8β. In some embodiments, the antibody or antigen binding fragment thereof binds to a human CD8α homodimer composed of two a chains. In some embodiments, the antibody or antigen binding fragment thereof binds to a human CD8 heterodimer composed of one a chain and one β chain.

In some embodiments, the antibody or antigen binding fragment binding CD8 is a single-chain variable fragment. In embodiments involving a single polypeptide containing both a heavy chain variable region and a light chain variable region, both orientations of these variable regions are contemplated. In some embodiments, the heavy chain variable region is on the N-terminal side of the light chain variable region, which means the heavy chain variable region is closer to the N-terminus of the polypeptide. In other embodiments, the light chain variable region is on the N-terminal side of the heavy chain variable region, which means the light chain variable region is closer to the N-terminus of the polypeptide than the heavy chain variable region.

In some embodiments, the scFv binding proteins comprise a linker. In some embodiments, the linker is between the heavy chain variable region (VH) and the light chain variable region (VL) (or vice versa). In some embodiments, the linker comprises the amino acid sequence of GS, GGS, GGGS (SEQ ID NO:227), GGGGS (SEQ ID NO:147), GGGGGS (SEQ ID NO:145), any one of SEQ ID NOs:165-166 and 32-33, or combinations thereof. Substitutions to introduce new disulfide bonds are also within the scope of the disclosure, e.g., by making substitutions G44C In the VH FR 2 and G100C in the VL FR4.

In some embodiments, the anti-CD8 antibody or antigen binding fragment binds to human CD8 with an affinity constant (K_{D}) of between about 1 nM and about 900 nM. In some embodiments, the K_{D} to human CD8 is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the anti-CD8 antibody or antigen binding fragment binds to human CD8 with an affinity constant (K_{D}) of 500 nM, 400 nM, 300 nM, 200 nM, 100 nM, 50 nM, 20 nM, or 10 nM or lower. In some embodiments, the anti-CD8 antibody or antigen binding fragment binds to human CD8 and cynomolgus, M. mulatta (rhesus monkey), or M. nemestrina CD8 with comparable binding affinity (K_{D}).

In some embodiments, the anti-CD8 antibody or antigen binding fragment binds to cynomolgus, M. mulatta (rhesus monkey), or N. nemestrina CD8. In some embodiments, the anti-CD8 antibody or antigen binding binds to mouse, dog, pig, etc., CD8. In some embodiments, the K_{D} to cynomolgus or M. nemestrina CD8 is between about 5 nM about 500 nM, about 6 nM to about 10 nM, about 11 nM to about 20 nM, about 25 nM to about 40 nM about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the anti-CD8 antibody or antigen binding fragment binds to cynomolgus or M. nemestrina CD8 with an affinity constant (K_{D}) of 500 nM, 400 nM, 300 nM, 200 nM, 100 nM, 50 nM, 20 nM, or 10 nM or lower.

An antibody or antigen binding fragment thereof that specifically binds CD8α or CD8β refers to an antibody or binding fragment that preferentially binds to CD8α or to CD8β, respectively, over other antigen targets. As used herein, the term is interchangeable with an “anti-CD8” antibody or an “antibody that binds CD8.” In some embodiments, the antibody or binding fragment capable of binding to CD8α or CD8β can do so with higher affinity for that antigen than others. In some embodiments, the antibody or binding fragment capable of binding CD8α or CD8β can bind to that antigen with a K_{D} of at least about 10⁻¹, 10⁻², 10⁻³, 10⁻⁴, 10⁻⁵, 10⁻⁶, 10⁻⁷, 10⁻⁸, 10⁻⁹, 10⁻¹⁰,10⁻¹¹, 10⁻¹² or greater (or any value in between), e.g., as measured by surface plasmon resonance or other methods known to those skilled in the art.

### C. Exogenous Agent

In some embodiments, the targeted vector further comprises an agent that is exogenous relative to the source cell (also referred to herein as a “cargo” or “payload”). In some embodiments, the exogenous agent is a small molecule, a protein, or a nucleic acid (e.g., a DNA, a chromosome (e.g., a human artificial chromosome), an RNA, e.g., an mRNA or miRNA). In some embodiments, the exogenous agent or cargo encodes a cytosolic protein. In some embodiments the exogenous agent or cargo comprises or encodes a membrane protein. In some embodiments, the exogenous agent or cargo comprises a therapeutic agent. In some embodiments, the therapeutic agent Is chosen from one or more of a protein, e.g., an enzyme, a transmembrane protein, a receptor, an antibody; a nucleic acid, e.g., DNA, a chromosome (e.g., a human artificial chromosome), RNA, mRNA, siRNA. miRNA; or a small molecule.

In some embodiments, the exogenous agent is present in at least, or no more than, 10, 20, 50. 100, 200, 500, 1,000, 2,000, 5,000, 10,000. 20,000, 50,000, 100,000, 200,000, 500,000, 1,000,000, 5,000,000, 10,000,000, 50,000.000, 100,000,000, 500,000.000, or 1,000,000,000 copies. In some embodiments, the targeted lipid particle has an altered. e.g., increased or decreased level of one or more endogenous molecules. e.g., protein or nucleic acid (e.g., in some embodiments, endogenous relative to the source cell, and in some embodiments, endogenous relative to the target cell), e.g., due to treatment of the source cell, e.g., mammalian source cell with a siRNA or gene editing enzyme. In some embodiments, the endogenous molecule is present in at least, or no more than, 10, 20, 50, 100, 200, 500, 1,000, 2,000, 5,000, 10,000, 20,000, 50,000, 100,000. 200,000, 500,000, 1,000,000, 5,000,000, 10,000,000, 50,000,000, 100,000,000, 500,000,000, or 1,000,000,000 copies. In some embodiments, the endogenous molecule (e.g., an RNA or protein) is present at a concentration of at least 1, 2, 3, 4, 5, 10. 20, 50, 100, 500, 10³, 5.0×10³, 10⁴, 5.0×10⁵, 10⁵, 5.0×10⁵, 10⁶, 5.0×10⁶, 1.0×10⁷, 5.0×10⁷, or 1.0×10⁶, greater than its concentration in the source cell. In some embodiments, the endogenous molecule (e.g., an RNA or protein) is present at a concentration of at least 1, 2, 3, 4, 5, 10, 20, 50, 100, 500, 10³, 5.0×10³, 10³, 5.0×10⁴, 10⁵, 5.0×10⁵, 10⁶, 5.0×10⁸, 1.0×10⁷, 5.0×10⁷, or 1.0×10⁸ less than its concentration in the source cell.

In some embodiments, the targeted lipid particle (e.g., targeted vector) delivers to a target cell at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the targeted lipid particle. In some embodiments, the targeted lipid particle that fuses with the target cell(s) delivers to the target cell an average of at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the targeted lipid particle that fuses with the target cell(s). In some embodiments, the targeted lipid particle composition delivers to a target tissue at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the targeted lipid particle composition.

In some embodiments, the exogenous agent or cargo is not expressed naturally in the cell from which the targeted lipid particle is derived. In some embodiments, the exogenous agent or cargo is expressed naturally in the cell from which the vector is derived. In some embodiments, the exogenous agent or cargo is loaded into the targeted lipid particle via expression in the cell from which the vector is derived (e.g., expression from DNA or mRNA introduced via transfection, transduction, or electroporation). In some embodiments, the exogenous agent or cargo is expressed from DNA integrated into the genome or maintained episosomally. In some embodiments, expression of the exogenous agent or cargo is constitutive. In some embodiments, expression of the exogenous agent or cargo is induced. In some embodiments, expression of the exogenous agent or cargo is induced immediately prior to generating the targeted lipid particle. In some embodiments, expression of the exogenous agent or cargo is induced at the same time as expression of the fusogen.

In some embodiments, the exogenous agent or cargo is loaded into the targeted lipid particle via electroporation into the targeted lipid particle itself or into the cell from which the targeted lipid particle is derived. In some embodiments, the exogenous agent or cargo is loaded into the targeted lipid particle via transfection (e.g., of a DNA or mRNA encoding the cargo) into the targeted lipid particle itself or into the cell from which the targeted lipid particle is derived.

In some embodiments, the exogenous agent or cargo may include one or more nucleic acid sequences, one or more polypeptides, a combination of nucleic acid sequences and/or polypeptides, one or more organelles, and any combination thereof. In some embodiments, the exogenous agent or cargo may include one or more cellular components. In some embodiments, the exogenous agent or cargo includes one or more cytosolic and/or nuclear components.

In some embodiments, the exogenous agent or cargo includes a nucleic acid, e.g., DNA, nDNA (nuclear DNA), mtDNA (mitochondrial DNA), protein coding DNA, gene, transgene, operon, chromosome, genome, transposon, retrotransposon, viral genome, vector, polycistronic vector, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), incRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprograming RNAs, aptamers, and any combination thereof. In some embodiments, the nucleic acid is a wild-type nucleic acid. In some embodiments, the nucleic acid is a mutant nucleic acid. In some embodiments the nucleic acid is a fusion or chimera of multiple nucleic acid sequences. 1o In some embodiments, the exogenous agent or cargo may include a nucleic acid.

For example, the exogenous agent or cargo may comprise RNA to enhance expression of an endogenous protein, or a siRNA or miRNA that inhibits protein expression of an endogenous protein. For example, the endogenous protein may modulate structure or function in the target cells. In some embodiments, the cargo may include a nucleic acid encoding an engineered protein that modulates structure or function in the target cells. In some embodiments, the exogenous agent or cargo is a nucleic acid that targets a transcriptional activator that modulate structure or function in the target cells.

In some embodiments, the exogenous agent or cargo includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides. metabolic polypeptides, kinases, transferases, hydrolases, lyases, isomer ases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g., Zinc-finger nucleases, transcription-activator-like nucleases (TALENs), cas9 and homologs thereof), recombinases, and any combination thereof. In some embodiments the protein targets a protein in the cell for degradation. In some embodiments the protein targets a protein in the cell for degradation by localizing the protein to the proteasome. In some embodiments, the protein is a wild-type protein. In some embodiments, the protein is a mutant protein. In some embodiments the protein is a fusion or chimeric protein.

In some embodiments, the exogenous agent or cargo includes a small molecule, e.g., ions (e.g., Ca²⁺, C1-, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degradation. In some embodiments the small molecule targets a protein in the cell for degradation by localizing the protein to the proteasome. In some embodiments that small molecule is a proteolysis targeting chimera molecule (PROTAC).

In some embodiments, the exogenous agent or cargo includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g., Cas9-gRNA complex); multiple transcription factors, multiple epigenetic factors, reprogramming factors (e.g., Oct4, Sox2, cMyc, and Klf4); multiple regulatory RNAs; and any combination thereof.

In some embodiments, the exogenous agent or cargo includes one or more organelles, e.g., chondrisomes, mitochondria, lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome. proteasome, vesicle, stress granule, networks of organelles, and any combination thereof.

In some embodiments, the exogenous agent encodes a therapeutic agent or a diagnostic agent. In some embodiments, the therapeutic agent is a chimeric antigen receptor (CAR). In some embodiments, the CAR specifically binds CD19 (e.g., the CAR comprises any of the antibodies or antigen binding fragments described herein). In some embodiments the CAR is bispecific and specifically binds CD19 and specifically binds one of CD5, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRa, IL-13Rα, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, G02, G_{D3}, HMW-MAA, IL-11Ra, KDR, Lewis Y, L1-cell adhesion molecule. MADE-A1, Oncofetal antigen (h5T4), TAG-72, CD19/22, Syndecan 1, or BCMA. In some embodiments, the CAR is engineered to comprise an intracellular signaling domain of the T cell antigen receptor complex zeta chain (e.g., CD3 zeta). In some embodiments, the intracellular domain is selected from a CD137 (4-1BB) signaling domain, a CD28 signaling domain, and a CD3zeta signaling domain.

### D. G Protein

Also provided herein are fusion proteins comprising an envelope glycoprotein G, H, and/or an F protein of the Paramyxoviridae family and a targeting antibody or antigen binding fragment thereof herein disclosed that are exposed on the surface on a lipid particle or viral vector. In some embodiments, the targeting antibody or antigen binding fragment thereof disclosed herein is fused to an envelope glycoprotein G, H. and/or an F protein of the Paramyxoviridae family. In some embodiments the fusogen contains a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein. In some embodiments, the lipid particle contains a henipavirus envelope attachment glycoprotein G (G protein) or a biologically active portion thereof and/or a henipavirus envelope fusion glycoprotein F (F protein) or a biologically active portion thereof.

In some embodiments, the fusogen is glycoprotein GP64 of baculovirus, or glycoprotein GP64 variant E45K/T259A.

In some embodiments, the fusogen is a hemagglutinin-neuraminidase (HN) and/or fusion (F) protein (F/HN) from a respiratory paramyxovirus. In some embodiments, the respiratory paramyxovirus is a Sendai virus. The HN and F glycoproteins of Sendai viruses function to attach to sialic acids via the HN protein, and to mediate cell fusion for entry into cells via the F protein. In some embodiments, the fusogen is a F and/or HN protein from the murine parainfluenza virus type 1 (see e.g., U.S. Pat. No. 10,704,061).

In some embodiments, the lipid particle (e.g., viral vector) is pseudotyped with viral glycoproteins as described herein such as a NiV—F and/or NiV-G protein.

In some embodiments, the viral vector further comprises a vector-surface targeting moiety which specifically binds to a target ligand. In some embodiments, the vector-surface targeting moiety is a polypeptide. In some embodiments, a nucleic acid encoding the Paramyxovirus envelope protein (e.g., G protein) is modified with a targeting moiety to specifically bind to a target molecule on a target cell. In some embodiments, the targeting moiety is any targeting protein, including but not necessarily limited to antibodies and antigen binding fragments thereof as herein disclosed.

It has been reported that the henipavirus F proteins from various species exhibit compatibility with G proteins from other species to trigger fusion (Brandel-Tretheway et al. Journal of Virology. 2019. 93(13):e00577-19). In some aspects of the provided lipid particles (e.g., lentiviral vectors), the F protein is heterologous to the G protein, i.e., the F and G proteins or biologically active portions thereof are from different henipavirus species. For example, in some embodiments the G protein is from Hendra virus and the F protein is a NiV-F as described. In other aspects, the F and/or G protein are chimeric F and/or G protein containing regions of F and/or G proteins from different species of Henipavirus. In some embodiments, replacing a portion of the F protein with amino acids from a heterologous sequence of Henipavirus results in fusion to the G protein with the heterologous sequence. (Brandel-Tretheway et al. 2019). In some embodiments, the chimeric F and/or G protein contains an extracellular domain from one henipavirus species and a transmembrane and/or cytoplasmic domain from a different henipavirus species. For example, in some embodiments the F protein contains an extracellular domain of Hendra virus and a transmembrane/cytoplasmic domain of Nipah virus.

In some embodiments, the fusion protein contains a henipavirus envelope attachment glycoprotein G (G protein) or a biologically active portion thereof and a single domain antibody (sdAb) variable domain or a single chain variable fragment (scFv). In some embodiments, the sdAb variable domain or scFv is linked directly or indirectly to the G protein. In some embodiments, the sdAb variable domain or scFv is linked to the C-terminus (C-terminal amino acid) of the G protein or the biologically active portion thereof. In some embodiments, the linkage is via a peptide linker, such as a flexible peptide linker. Table 26 provides a list of non-limiting examples of G proteins.

In some embodiments the G protein is a Henipavirus G protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein, or a biologically active portion thereof. Non-limiting examples of G proteins include those corresponding to SEQ ID NOs: 129, 138, 139, 140, and 141.

In some embodiments, the attachment G proteins are type II transmembrane glycoproteins containing an N-terminal cytoplasmic tail (e.g., corresponding to amino adds 1-49 of SEQ ID NO: 120), a transmembrane domain (e.g., corresponding to amino acids 50-70 of SEQ ID NO: 120), and an extracellular domain containing an extracellular stalk (e.g., corresponding to amino acids 71-187 of SEQ ID NO: 120), and a globular head (corresponding to amino acids 188-602 of SEQ ID NO: 120). In such embodiments, the N-terminal cytoplasmic domain is within the inner lumen of the lipid bilayer and the C-terminal portion is the extracellular domain that is exposed on the outside of the lipid bilayer. Regions of the stalk in the C-terminal region (e.g., corresponding to amino acids 159-167 of NiV-G) have been shown to be involved in interactions with F protein and triggering of F protein fusion (Liu et al. 2015 J of Virology 89:1838). In wild-type G protein, the globular head mediates receptor binding to henipavirus entry receptors ephrin B2 and ephrin B3, but is dispensable for membrane fusion (Brandel-Tretheway et al. Journal of Virology. 2019. 93(13)e00577-19). In some embodiments herein, tropism of the G protein is altered by linkage of the G protein or biologically active fragment thereof (e.g., cytoplasmic truncation) to a sdAb variable domain. Binding of the G protein to a binding partner can trigger fusion mediated by a compatible F protein or a biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved among henipavirus species. For example, the G proteins of NiV and HeV viruses share 79% amino acid identity. Studies have shown a high degree of compatibility among G proteins with F proteins of different species as demonstrated by heterotypic fusion activation (Brandel-Tretheway et al. Journal of Virology. 2019). As described further below, in some embodiments, a targeted lipid particle contains heterologous G and F proteins from different species.

In some embodiments, the G protein has a sequence set forth in any of SEQ ID NOs: 120, 129, 138, 139, 140, 141, 148, 156, or 158-160, or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOs: 120, 129, 138, 139, 140, 141, 148, 156, or 158-160. In some embodiments, the G protein or functionally active variant or biologically active portion is a protein that retains fusogenic activity in conjunction with a Henipavirus F protein, such as an F protein (e.g., NiV—F or HeV-F). Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g., a cell that contains a surface receptor or molecule that is recognized or bound by the targeted lipid particle. In some embodiments, the F protein and G protein are from the same Henipavirus species (e.g., NiV-G and NiV-F). In some embodiments, the F protein and G protein are from different Henipavirus species (e.g., NiV-G and HeV-F).

In some embodiments, the G protein has the sequence of amino acids set forth in SEQ ID NOs: 120, 129, 138, 139, 140, 141, 148, 156, or 158-160, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to any one of SEQ ID NOs: 120, 129, 138, 139, 140, 141, 148, 156, or 158-160and retains fusogenic activity in conjunction with a Henipavirus F protein (e.g., NiV—F or HeV-F). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to any one of SEQ ID NOs: 120, 129, 138, 139, 140, 141, 148, 156, or 158-160and retains fusogenic activity in conjunction with a Henipavirus F protein (e.g., NiV—F or HeV-F).

Reference to retaining fusogenic activity includes activity (in conjunction with a Henipavirus F protein) that is at or about 10% to at or about 150% or more of the level or degree of binding of the corresponding wild-type G protein, such as set forth in any one of SEQ ID NOs: 120, 129, 138, 139, 140, 141, 148, 156, or 158-160, such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 80% of the level or degree of fusogenic activity of the corresponding wild-type G protein, such as at least or at least about 85% of the level or degree of fusogenic activity of the corresponding wild-type G protein, such as at least or at least about 90% of the level or degree of fusogenic activity of the corresponding wild-type G protein, such as at least or at least about 95% of the level or degree of fusogenic activity of the corresponding wild-type G protein, such as at least or at least about 100% of the level or degree of fusogenic activity of the corresponding wild-type G protein, or such as at least or at least about 120% of the level or degree of fusogenic activity of the corresponding wild-type G protein.

In some embodiments, the G protein is a mutant G protein that is a functionally active variant or biologically active portion containing one or more amino acid mutations, such as one or more amino acid insertions, deletions, substitutions, or truncations. In some embodiments, the mutations described herein relate to amino acid insertions, deletions, substitutions, or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NIV) virus G-protein (NiV-G), a wild-type Cedar (CedPV) virus G-protein, a wild-type Mojiang virus G-protein, a wild-type bat Paramyxovirus G-protein, or biologically active portions thereof. In some embodiments, the wild-type G protein has the sequence set forth in any one of SEQ ID NOs: 120, 129, 138, 139, 140, 141. 148, 158, or 158-160.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, or a wild-type bat Paramyxovirus G-protein. In some embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NOs: 120, 129, 138, 139, 140, 141, 148, 156, or 158-160. In some embodiments, the mutant G protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25. 24. 23, 22, 21, 20, 19, 18, 17, 16, 15, 14, 13, 12, 11, 10, 9, 8, 7, 6, 5, 4, 3, 2 or 1 contiguous amino acid(s) at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:120, SEQ ID NO:138, or SEQ ID NO:148, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:120. SEQ ID NO:138, or SEQ ID NO:148.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein (SEQ ID NO:120, SEQ ID NO:138. or SEQ ID NO:148), up to 6 contiguous amino acid residues at or near the N-terminus of the wild-type NiV-G protein (SEQ ID NO:120. SEQ ID NO:138, or SEQ ID NO:148), up to 7 contiguous amino acid residues at or near the N-terminus of the wild-type NiV-G protein (SEQ ID NO:120, SEQ ID NO:138, or SEQ ID NO:148), up to 8 contiguous amino acid residues at or near the N-terminus of the wild-type NiV-G protein (SEQ ID NO:120, SEQ ID NO:138, or SEQ ID NO:148), up to 9 contiguous amino acid residues at or near the N-terminus of the wild-type NiV-G protein (SEQ ID NO:120, SEQ ID NO:138, or SEQ ID NO:148). up to 10 contiguous amino acid residues at or near the N-terminus of the wild-type NiV-G protein (SEQ ID NO:120, SEQ ID NO:138, or SEQ ID NO:148), up to 11 contiguous amino acid residues at or near the N-terminus of the wild-type NiV-G protein (SEQ ID NO:120, SEQ ID NO:138, or SEQ ID NO:148), up to 12 contiguous amino acid residues at or near the N-terminus of the wild-type NiV-G protein (SEQ ID NO:120, SEQ ID NO:138, or SEG ID NO:148), up to 13 contiguous amino acid residues at or near the N-terminus of the wild-type NiV-G protein (SEQ ID NO:120, SEQ ID NO:138, or SEQ ID NO:148), up to 14 contiguous amino acid residues at or near the N-terminus of the wild-type NiV-G protein (SEQ ID NO:120, SEQ ID NO:138, or SEQ ID NO:148), up to 15 contiguous amino acid residues at or near the N-terminus of the wild-type NiV-G protein (SEQ ID NO:120, SEQ ID NO:138, or SEQ ID NO:148), up to 16 contiguous amino acid residues at or near the N-terminus of the wild-type NiV-G protein (SEQ ID NO:120, SEQ ID NO:138, or SEQ ID NO:148), up to 17 contiguous amino acid residues at or near the N-terminus of the wild-type NiV-G protein (SEQ ID NO:120, SEQ ID NO:138, or SEQ ID NO:148), up to 18 contiguous amino acid residues at or near the N-terminus of the wild-type NiV-G protein (SEQ ID NO:120, SEQ ID NO:138. or SEQ ID NO:148), up to 19 contiguous amino acid residues at or near the N-terminus of the wild-type NiV-G protein (SEQ ID NO:120, SEQ ID NO:138, or SEQ ID NO:148), up to 20 contiguous amino acid residues at or near the N-terminus of the wild-type NiV-G protein (SEQ ID NO:120. SEQ ID NO:138, or SEQ ID NO:148), up to 21 contiguous amino acid residues at or near the N-terminus of the wild-type NiV-G protein (SEQ ID NO:120, SEQ ID NO:138, or SEQ ID NO:148), up to 22 contiguous amino acid residues at or near the N-terminus of the wild-type NiV-G protein (SEQ ID NO:120, SEQ ID NO:138, or SEQ ID NO:148), up to 23 contiguous amino acid residues at or near the N-terminus of the wild-type NiV-G protein (SEQ ID NO:120, SEQ ID NO:138, or SEQ ID NO:148), up to 24 contiguous amino acid residues at or near the N-terminus of the wild-type NiV-G protein (SEQ ID NO:120, SEQ ID NO:138. or SEQ ID NO:148), up to 25 contiguous amino acid residues at or near the N-terminus of the wild-type NiV-G protein (SEQ ID NO:120, SEQ ID NO:138, or SEQ ID NO:148), up to 26 contiguous amino acid residues at or near the N-terminus of the wild-type NiV-G protein (SEQ ID NO:120, SEQ ID NO:138, or SEQ ID NO:148), up to 27 contiguous amino acid residues at or near the N-terminus of the wild-type NiV-G protein (SEQ ID NO:120. SEQ ID NO:138, or SEQ ID NO:148), up to 28 contiguous amino acid residues at or near the N-terminus of the wild-type NiV-G protein (SEQ ID NO:120, SEQ ID NO:138, or SEQ ID NO:148), up to 29 contiguous amino acid residues at or near the N-terminus of the wild-type NiV-G protein (SEQ ID NO:120, SEQ ID NO:138, or SEQ ID NO:148), up to 30 contiguous amino acid residues at or near the N-terminus of the wild-type NiV-G protein (SEQ ID NO:120, SEQ ID NO:138, or SEQ ID NO:148). up to 31 contiguous amino acid residues at or near the N-terminus of the wild-type NiV-G protein (SEQ ID NO:120, SEQ ID NO:138, or SEQ ID NO:148), up to 32 contiguous amino acid residues at or near the N-terminus of the wild-type NiV-G protein (SEQ ID NO:120. SEQ ID NO:138, or SEQ ID NO:148), up to 33 contiguous amino acid residues at or near the N-terminus of the wild-type NiV-G protein (SEQ ID NO:120, SEQ ID NO:138, or SEQ ID NO:148), up to 34 contiguous amino acid residues at or near the N-terminus of the wild-type NIV-G protein (SEQ ID NO:120, SEQ ID NO:138, or SEQ ID NO:148), up to 35 contiguous amino acid residues at or near the N-terminus of the wild-type NiV-G protein (SEQ ID NO:120, SEQ ID NO:138, or SEQ ID NO:148), up to 36 contiguous amino acid residues at or near the N-terminus of the wild-type NIV-G protein (SEQ ID NO:120, SEQ ID NO:138, or SEQ ID NO-148), up to 37 contiguous amino acid residues at or near the N-terminus of the wild-type NiV-G protein (SEQ ID NO:120, SEQ ID NO:138, or SEQ ID NO:148), up to 38 contiguous amino acid residues at or near the N-terminus of the wild-type NiV-G protein (SEQ ID NO:120, SEQ ID NO:138. or SEQ ID NO:148), up to 39 contiguous amino acid residues at or near the N-terminus of the wild-type NiV-G protein (SEQ ID NO:120, SEQ ID NO:138, or SEQ ID NO:148), up to 40 contiguous amino acid residues at or near the N-terminus of the wild-type NiV-G protein (SEQ ID NO:120. SEQ ID NO:138, or SEQ ID NO:148), up to 41 contiguous amino acid residues at or near the N-terminus of the wild-type NiV-G protein (SEQ ID NO:120, SEQ ID NO:138, or SEQ ID NO:148), up to 42 contiguous amino acid residues at or near the N-terminus of the wild-type NiV-G protein (SEQ ID NO:120, SEQ ID NO:138, or SEQ ID NO:148), up to 43 contiguous amino acid residues at or near the N-terminus of the wild-type NiV-G protein (SEQ ID NO:120, SEQ ID NO:138, or SEQ ID NO:148), up to 44 contiguous amino acid residues at or near the N-terminus of the wild-type NiV-G protein (SEQ ID NO:120, SEQ ID NO: 138, or SEQ ID NO: 148). or up to 45 contiguous amino acid residues at or near the N-terminus of the wild-type NiV-G protein (SEQ ID NO:120, SEQ ID NO:138, or SEQ ID NO:148).

In some embodiments, the NiV-G protein is a biologically active portion that does not contain a cytoplasmic domain. In some embodiments, the NiV-G protein without the cytoplasmic domain is encoded by SEQ ID NO:142.

In some embodiments, the mutant NiV-G protein comprises a sequence set forth in any of SEQ ID NOs: 121-126, 149-154, 132, 142, or 157, or is a functional variant thereof that has an amino acid sequence having at least at or about 80%, at least at or about 81%, at least at or about 82%, at least at or about 83%, at least at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NOs: 121-128, 149-154, 132, 142, or 157.

In some embodiments, the mutant NiV-G protein has a 5 amino acid truncation at or near the N-terminus of the wild-type NiV-G protein (SEQ ID NO:120, SEQ ID NO:138, or SEQ ID NO:148), such as set forth in SEQ ID NO:121 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:121, or as set forth in SEQ ID NO:149 or afunctional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:121 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:149.

In some embodiments, the mutant NiV-G protein has a 10 amino acid truncation at or near the N-terminus of the wild-type NiV-G protein (SEQ ID NO:120, SEQ ID NO:138, or SEQ ID NO:148), such as set forth in SEQ ID NO:122 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:122, or such as set forth in SEQ ID NO:150 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:150.

In some embodiments, the mutant NiV-G protein has a 15 amino acid truncation at or near the N-terminus of the wild-type NiV-G protein (SEQ ID NO:120, SEQ ID NO:138, or SEQ ID NO:148), such as set forth in SEQ ID NO:123 or a functional variant thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:123, or such as set forth in SEQ ID NO:151 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:151.

In some embodiments, the mutant NiV-G protein has a 20 amino acid truncation at or near the N-terminus of the wild-type NiV-G protein (SEQ ID NO:120, SEQ ID NO:138, or SEQ ID NO:148) such as set forth in SEQ ID NO:124, or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least or about 96%, at least at or about 97%, at least at or about 98%, or at least at or about 99% sequence identity to SEQ ID NO:124, or such as set forth in SEQ ID NO:152 or afunctional variant thereof having at least at or about 80%, at least at or about 81%, at least at or about 82%, at least at or about 83%, at least or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:152.

In some embodiments, the mutant NiV-G protein has a 25 amino acid truncation at or near the N-terminus of the wild-type NiV-G protein (SEQ ID NO:120, SEQ ID wo NO:138, or SEQ ID NO:148), such as set forth in SEQ ID NO:125 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least or about 96%, at least at or about 97%, at least at or about 98%, or at least at or about 99% sequence identity to SEQ ID NO:125, or such as set forth in SEQ ID NO:153 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:153.

In some embodiments, the mutant NiV-G protein has a 30 amino acid truncation at or near the N-terminus of the wild-type NiV-G protein (SEQ ID NO:120, SEQ ID NO:138, or SEQ ID NO:148), such as set forth in SEQ ID NO:126 or a functional variant thereof having at least at or about 80%, at least at or about 81%, at least at or about 82%, at least at or about 83%, at least at or about 84%, at least at or about 85%, at least at or about 88%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:126, or such as set forth in SEQ ID NO:154 or afunctional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:154.

In some embodiments, the mutant NiV-G protein has a 33 amino acid truncation at or near the N-terminus of the wild-type NiV-G protein (SEQ ID NO:120, SEQ ID NO:138, or SEQ ID NO:148) or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:132, or such as set forth in SEQ ID NO:155 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:155.

n some embodiments, the mutant NiV-G protein has a 34 amino acid truncation at or near the N-terminus of the wild-type NiV-G protein (SEQ ID NO:120, SEQ ID NO:138, or SEQ ID NO:148), such as set forth in SEQ ID NO:132 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:132. or such as set forth in SEQ ID NO:155 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:155.

In some embodiments, the NiV-G protein has a 34 amino acid truncation at or near the N-terminus of the wild-type NiV-G protein (SEQ ID NO: 120, SEQ ID NO: 138, or SEQ ID NO: 148) and one or more amino acid substitutions corresponding to amino acid substitutions selected from E501A, W504A, Q530A, and E533A with reference to the numbering set forth in SEQ ID NO: 138.

In some embodiments, the mutant NiV-G protein lacks the N-terminal cytoplasmic domain of the wild-type NiV-G protein (SEQ ID NO:120, SEQ ID NO:138, or SEQ ID NO:148), such as set forth in SEQ ID NO:142 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:142.

In some embodiments, the mutant G protein is a mutant HeV-G protein that has the sequence set forth in SEQ ID NO:129 or 156, or is a functional variant or biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least or about 96%, at least at or about 97%, at least at or about 98%, or at least at or about 99% sequence identity to SEQ ID NO:129 or 156.

In some embodiments, the G protein is a mutant HeV-G protein that is a biologically active portion of a wild-type HeV-G. In some embodiments, the biologically active portion is an N-terminally truncated fragment. In some embodiments, the mutant HeV-G protein is truncated and lacks up to 5 contiguous amino acid residues at or near the N-terminus of the wild-type HeV-G protein (SEQ ID NO:129 or 156), up to 6 contiguous amino acid residues at or near the N-terminus of the wild-type HeV-G protein (SEQ ID NO:129 or 156), up to 7 contiguous amino acid residues at or near the N-terminus of the wild-type HeV-G protein (SEQ ID NO:129 or 156), up to 8 contiguous amino acid residues at or near the N-terminus of the wild-type HeV-G protein (SEQ ID NO:129 or 156). up to 9 contiguous amino acid residues at or near the N-terminus of the wild-type HeV-G protein (SEQ ID NO:129 or 156), up to 10 contiguous amino acid residues at or near the N-terminus of the wild-type HeV-G protein (SEQ ID NO:129 or 156), up to 11 contiguous amino acid residues at or near the N-terminus of the wild-type HeV-G protein (SEQ ID NO:129 or 156), up to 12 contiguous amino acid residues at or near the N-terminus of the wild-type HeV-G protein (SEQ ID NO:129 or 156), up to 13 contiguous amino acid residues at or near the N-terminus of the wild-type HeV-G protein (SEQ ID NO:129 or 156), up to 14 contiguous amino acid residues at or near the N-terminus of the wild-type HeV-G protein (SEQ ID NO:129 or 156), up to 15 contiguous amino acid residues at or near the N-terminus of the wild-type HeV-G protein (SEQ ID NO:129 or 156), up to 16 contiguous amino acid residues at or near the N-terminus of the wild-type HeV-G protein (SEQ ID NO:129 or 156). up to 17 contiguous amino acid residues at or near the N-terminus of the wild-type HeV-G protein (SEQ ID NO:129 or 156), up to 18 contiguous amino acid residues at or near the N-terminus of the wild-type HeV-G protein (SEQ ID NO:129 or 156), up to 19 contiguous amino acid residues at or near the N-terminus of the wild-type HeV-G protein (SEQ ID NO:129 or 156), up to 20 contiguous amino acid residues at or near the N-terminus of the wild-type HeV-G protein (SEQ ID NO:129 or 156), up to 21 contiguous amino acid residues at or near the N-terminus of the wild-type HeV-G protein (SEQ ID NO:129 or 156), up to 22 contiguous amino acid residues at or near the N-terminus of the wild-type HeV-G protein (SEQ ID NO:129 or 156), up to 23 contiguous amino acid residues at or near the N-terminus of the wild-type HeV-G protein (SEQ ID NO:129 or 156), up to 24 contiguous amino acid residues at or near the N-terminus of the wild-type HeV-G protein (SEQ ID NO:129 or 156), up to 25 contiguous amino acid residues at or near the N-terminus of the wild-type HeV-G protein (SEQ ID NO:129 or 156), up to 26 contiguous amino acid residues at or near the N-terminus of the wild-type HeV-G protein (SEQ ID NO:129 or 156), up to 27 contiguous amino acid residues at or near the N-terminus of the wild-type HeV-G protein (SEQ ID NO:129 or 156), up to 28 contiguous amino acid residues at or near the N-terminus of the wild-type HeV-G protein (SEQ ID NO:129 or 156), up to 29 contiguous amino acid residues at or near the N-terminus of the wild-type HeV-G protein (SEQ ID NO:129 or 156), up to 30 contiguous amino acid residues at or near the N-terminus of the wild-type HeV-G protein (SEQ ID NO:129 or 156), up to 31 contiguous amino acid residues at or near the N-terminus of the wild-type HeV-G protein (SEQ ID NO:129 or 156), up to 32 contiguous amino acid residues at or near the N-terminus of the wild-type HeV-G protein (SEQ ID NO:129 or 156), up to 33 contiguous amino acid residues at or near the N-terminus of the wild-type HeV-G protein (SEQ ID NO:129 or 156), up to 34 contiguous amino acid residues at or near the N-terminus of the wild-type HeV-G protein (SEQ ID NO:129 or 156), up to 35 contiguous amino acid residues at or near the N-terminus of the wild-type HeV-G protein (SEQ ID NO:129 or 156), up to 36 contiguous amino acid residues at or near the N-terminus of the wild-type HeV-G protein (SEQ ID NO:129 or 156), up to 37 contiguous amino acid residues at or near the N-terminus of the wild-type HeV-G protein (SEQ ID NO:129 or 156), up to 38 contiguous amino acid residues at or near the N-terminus of the wild-type HeV-G protein (SEQ ID NO:129 or 156), up to 39 contiguous amino acid residues at or near the N-terminus of the wild-type HeV-G protein (SEQ ID NO:129 or 156), up to 40 contiguous amino acid residues at or near the N-terminus of the wild-type HeV-G protein (SEQ ID NO:129 or 156), up to 41 contiguous amino acid residues at or near the N-terminus of the wild-type HeV-G protein (SEQ ID NO:129 or 156), up to 42 contiguous amino acid residues at or near the N-terminus of the wild-type HeV-G protein (SEQ ID NO:129 or 156), up to 43 contiguous amino acid residues at or near the N-terminus of the wild-type HeV-G protein (SEQ ID NO:129 or 156), up to 44 contiguous amino acid residues at or near the N-terminus of the wild-type HeV-G protein (SEQ ID NO:129 or 156), or up to 45 contiguous amino acid residues at or near the N-terminus of the wild-type HeV-G protein (SEQ ID NO:129 or 156).

In some embodiments, the HeV-G protein is a biologically active portion that does not contain a cytoplasmic domain. In some embodiments, the mutant HeV-G protein lacks the N-terminal cytoplasmic domain of the wild-type HeV-G protein (SEQ ID NO:129 or 156), such as set forth in SEQ ID NO:143 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:143.

In some embodiments, the G protein or the functionally active variant or biologically active portion thereof binds to Ephrin B2 or Ephrin B3. In some aspects, the G protein has the sequence of amino acids set forth in any one of SEQ ID NO:120, SEQ ID NO:129, SEQ ID NO:138, SEQ ID NO:139, SEQ ID NO:148, SEQ ID NO:140, or SEQ ID NO:141, or Is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, and retains binding to Ephrin B2 or B3.

Reference to retaining binding to Ephrin B2 or B3 includes binding that is at least or at least about 5% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO-141, or a functionally active variant or biologically active portion thereof, 10% of the level or degree of binding of the corresponding wild-type G protein, such as set forth in SEQ ID NO:120. SEQ ID NO:129, SEQ ID NO:138, SEQ ID NO:139. SEQ ID NO:148, SEQ ID NO:140, or SEQ ID NO:141, or a functionally active variant or biologically active portion thereof, 15% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof. 20% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 25% of the level or degree of binding of the corresponding wild-type G protein, such as set forth in SEQ ID NO:120, SEQ ID NO:129, SEQ ID NO:138, SEQ ID NO:139, SEQ ID NO:148. SEQ ID NO:140, or SEQ ID NO:141, or a functionally active variant or biologically active portion, 30% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof. 35% of the level or degree of binding of the corresponding wild-type G protein, such as set forth in SEQ ID NO:120, SEQ ID NO:129. SEQ ID NO:138, SEQ ID NO:139, SEQ ID NO:148, SEQ ID NO:140, or SEQ ID NO:141, or a functionally active variant or biologically active portion thereof, 40% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 45% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 50% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 55% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 60% of the level or degree of binding of the corresponding wild-type G protein, such as set forth in SEO ID NO:120, SEQ ID NO:129, SEQ ID NO:138. SEQ ID NO:139, SEQ ID NO:148, SEQ ID NO:140, or SEQ ID NO:141, or a functionally active variant or biologically active portion thereof, 65% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 70% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 75% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140. or SEQ ID NO:141, or a functionally active variant or biologically active portion thereof, such as at least or at least about 80% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 85% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 90% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, or such as at least or at least about 95% of the level or degree of binding of the corresponding wild-type protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof.

In some embodiments, the G protein is NiV-G or a functionally active variant or biologically active portion thereof and binds to Ephrin B2 or Ephrin B3. In some aspects, the NiV-G has the sequence of amino acids set forth in SEQ ID NO:120, SEQ ID NO:138, or SEQ ID NO:148, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:120, SEQ ID NO:138, or SEQ ID NO:148 and retains binding to Ephrin B2 or B3. Exemplary biologically active portions include N-terminally truncated variants lacking all or a portion of the cytoplasmic domain, e.g., 1 or more, such as 1 to 49 contiguous N-terminal amino acid residues, e.g., set forth in any one of SEQ ID NOs: 121-126, 142, and 149-154.

Reference to retaining binding to Ephrin B2 or B3 includes binding that is at least or at least about 5% of the level or degree of binding of the corresponding wild-type NiV-G, such as set forth in SEQ ID NO:120, SEQ ID NO:138, or SEQ ID NO:148, 10% of the level or degree of binding of the corresponding wild-type NiV-G, such as set forth in SEQ ID NO:120, SEQ ID NO:138, or SEQ ID NO:148, 15% of the level or degree of binding of the corresponding wild-type NiV-G, such as set forth in SEQ ID NO:120, SEQ ID NO:138, or SEQ ID NO:148, 20% of the level or degree of binding of the corresponding wild-type NiV-G. such as set forth in SEQ ID NO:120, SEQ ID NO:138, or SEQ ID NO:148, 25% of the level or degree of binding of the corresponding wild-type NiV-G, such as set forth in SEQ ID NO:120. SEQ ID NO:138, or SEQ ID NO:148, 30% of the level or degree of binding of the corresponding wild-type NiV-G, such as set forth in S SEQ ID NO:120. SEQ ID NO:138, or SEQ ID NO:148, 35% of the level or degree of binding of the corresponding wild-type NiV-G, such as set forth in SEQ ID NO:120, SEQ ID NO:138, or SEQ ID NO:148, 40% of the level or degree of binding of the corresponding wild-type NiV-G, such as set forth in SEQ ID NO:120, SEQ ID NO:138, or SEQ ID NO: 148, 45% of the level or degree of binding of the corresponding wild-type NiV-G, such as set forth in SEQ ID NO:120. SEQ ID NO:138, or SEQ ID NO:148, 50% of the level or degree of binding of the corresponding wild-type NiV-G, such as set forth in SEQ ID NO:120, SEQ ID NO:138, or SEQ ID NO:148, 55% of the level or degree of binding of the corresponding wild-type NiV-G, such as set forth in SEQ ID NO:120, SEQ ID NO:138, or SEQ ID NO:148, 60% of the level or degree of binding of the corresponding wild-type NiV-G, such as set forth in SEQ ID NO:120, SEQ ID NO:138, or SEQ ID NO:148, 65% of the level or degree of binding of the corresponding wild-type NiV-G, such as set forth in SEQ ID NO:120, SEQ ID NO:138. or SEQ ID NO:148, 70% of the level or degree of binding of the corresponding wild-type NiV-G, such as set forth in SEQ ID NO:120, SEQ ID NO:138, or SEQ ID NO:148, such as at least or at least about 75% of the level or degree of binding of the corresponding wild-type NiV-G, such as set forth in SEQ ID NO:120, SEQ ID NO:138, or SEQ ID NO:148, such as at least or at least about 80% of the level or degree of binding of the corresponding wild-type NIV-G, such as set forth in SEQ ID NO:120, SEQ ID NO:138, or SEQ ID NO:148, such as at least or at least about 85% of the level or degree of binding of the corresponding wild-type NiV-G, such as set forth in SEQ ID NO:120, SEQ ID NO:138, or SEQ ID NO:148, such as at least or at least about 90% of the level or degree of binding of the corresponding wild-type NiV-G, such as set forth in SEQ ID NO:120, SEQ ID NO:138, or SEQ ID NO:148, or such as at least or at least about 95% of the level or degree of binding of the corresponding wild-type NiV-G, such as set forth in SEQ ID NO:120, SEQ ID NO:138. or SEQ ID NO:148.

In some embodiments, the G protein is HeV-G or a functionally active variant or biologically active portion thereof and binds to Ephrin B2 or Ephrin B3. In some aspects, the HeV-G has the sequence of amino acids set forth in SEQ ID NO:129 or 156, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:129 or 156 and retains binding to Ephrin B2 or B3. Exemplary biologically active portions include N-terminally truncated variants lacking all or a portion of the cytoplasmic domain, e.g., 1 or more, such as 1 to 49 contiguous N-terminal amino acid residues, e.g., set forth in any one of SEQ ID NO:143.

Reference to retaining binding to Ephrin B2 or B3 includes binding that is at least or at least about 5% of the level or degree of binding of the corresponding wild-type to HeV-G, such as set forth in SEQ ID NO:129 or 156, 10% of the level or degree of binding of the corresponding wild-type HeV-G, such as set forth in SEQ ID NO:129 or 156, 15% of the level or degree of binding of the corresponding wild-type HeV-G, such as set forth in SEQ ID NO:129 or 156, 20% of the level or degree of binding of the corresponding wild-type HeV-G, such as set forth in SEQ ID NO:129 or 156, 25% is of the level or degree of binding of the corresponding wild-type HeV-G, such as set forth in SEQ ID NO:129 or 156, 30% of the level or degree of binding of the corresponding wild-type HeV-G, such as set forth in SEQ ID NO:129 or 156, 35% of the level or degree of binding of the corresponding wild-type HeV-G, such as set forth in SEQ ID NO:129 or 156, 40% of the level or degree of binding of the corresponding wild-type HeV-G, such as set forth in SEQ ID NO:129 or 156, 45% of the level or degree of binding of the corresponding wild-type HeV-G, such as set forth in SEQ ID NO:129 or 156, 50% of the level or degree of binding of the corresponding wild-type HeV-G, such as set forth in SEQ ID NO:129 or 156, 55% of the level or degree of binding of the corresponding wild-type HeV-G, such as set forth in SEQ ID NO:129 or 156, 60% of the level or degree of binding of the corresponding wild-type HeV-G, such as set forth in SEQ ID NO:129 or 156, 65% of the level or degree of binding of the corresponding wild-type HeV-G, such as set forth in SEQ ID NO:129 or 156, 70% of the level or degree of binding of the corresponding wild-type HeV-G, such as set forth in SEQ ID NO:129 or 156, such as at least or at least about 75% of the level or degree of binding of the corresponding wild-type HeV-G, such as set forth in SEQ ID NO:129 or 156, such as at least or at least about 80% of the level or degree of binding of the corresponding wild-type NIV-G, such as set forth in SEQ ID NO:129 or 156, such as at least or at least about 85% of the level or degree of binding of the corresponding wild-type HeV-G, such as set forth in SEQ ID NO:129 or 156, such as at least or at least about 90% of the level or degree of binding of the corresponding wild-type HeV-G, such as set forth in SEQ ID NO:129 or 156, or such as at least or at least about 95% of the level or degree of binding of the corresponding wild-type HeV-G, such as set forth in SEQ ID NO:129 or 156.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such as to reduce the binding to at least one of Ephrin B2 or Ephrin B3. In some embodiments, the mutations described herein interfere with natural receptor recognition.

In some embodiments, the mutant NIV-G protein or the biologically active portion thereof is truncated and lacks up to 5 contiguous amino acid residues at or near the N-terminus of the wild-type NiV-G protein (SEQ ID NO:138), 6 contiguous amino acid residues at or near the N-terminus of the wild-type NiV-G protein (SEQ ID NO:138), 7 contiguous amino acid residues at or near the N-terminus of the wild-type NiV-G protein (SEQ ID NO:138), 8 contiguous amino acid residues at or near the N-terminus of the wild-type NiV-G protein (SEQ ID NO:138), 9 contiguous amino acid residues at or near the N-terminus of the wild-type NiV-G protein (SEQ ID NO:138), contiguous amino acid residues at or near the N-terminus of the wild-type NiV-G protein (SEQ ID NO:138), 11 contiguous amino acid residues at or near the N-terminus of the wild-type NiV-G protein (SEQ ID NO:138), 12 contiguous amino acid residues at or near the N-terminus of the wild-type NiV-G protein (SEQ ID NO:138), 13 contiguous amino acid residues at or near the N-terminus of the wild-type NiV-G protein (SEQ ID NO:138), 14 contiguous amino acid residues at or near the N-terminus of the wild-type NiV-G protein (SEQ ID NO:138), 15 contiguous amino acid residues at or near the N-terminus of the wild-type NiV-G protein (SEQ ID NO:138), 16 contiguous amino acid residues at or near the N-terminus of the wild-type NiV-G protein (SEQ ID NO:138), 17 contiguous amino acid residues at or near the N-terminus of the wild-type NiV-G protein (SEQ ID NO:138), 18 contiguous amino acid residues at or near the N-terminus of the wild-type NiV-G protein (SEQ ID NO:138), 19 contiguous amino acid residues at or near the N-terminus of the wild-type NiV-G protein (SEQ ID NO:138), 20 contiguous amino acid residues at or near the N-terminus of the wild-type NiV-G protein (SEQ ID NO:138), 21 contiguous amino acid residues at or near the N-terminus of the wild-type NiV-G protein (SEQ ID NO:138), 22 contiguous amino acid residues at or near the N-terminus of the wild-type NiV-G protein (SEQ ID NO:138), 23 contiguous amino acid residues at or near the N-terminus of the wild-type NiV-G protein (SEQ ID NO:138), 24 contiguous amino acid residues at or near the N-terminus of the wild-type NiV-G protein (SEQ ID NO:138), contiguous amino acid residues at or near the N-terminus of the wild-type NiV-G protein (SEQ ID NO:138), 26 contiguous amino acid residues at or near the N-terminus of the wild-type NiV-G protein (SEQ ID NO:138), 27 contiguous amino acid residues at or near the N-terminus of the wild-type NiV-G protein (SEQ ID NO:138), 28 contiguous amino acid residues at or near the N-terminus of the wild-type NiV-G protein (SEQ ID NO:138), 29 contiguous amino acid residues at or near the N-terminus of the wild-type NiV-G protein (SEQ ID NO:138), 30 contiguous amino acid residues at or near the N-terminus of the wild-type NIV-G protein (SEQ ID NO:138), 31 contiguous amino acid residues at or near the N-terminus of the wild-type NiV-G protein (SEQ ID NO:138), 32 contiguous amino acid residues at or near the N-terminus of the wild-type NiV-G protein (SEQ ID NO:138), 33 contiguous amino acid residues at or near the N-terminus of the wild-type NiV-G protein (SEQ ID NO:138), 34 contiguous amino acid residues at or near the N-terminus of the wild-type NiV-G protein (SEQ ID NO:138), 35 contiguous amino acid residues at or near the N-terminus of the wild-type NiV-G protein (SEQ ID NO:138), 36 contiguous amino acid residues at or near the N-terminus of the wild-type NiV-G protein (SEQ ID NO:138), 37 contiguous amino acid residues at or near the N-terminus of the wild-type NiV-G protein (SEQ ID NO:138). 38 contiguous amino add residues at or near the N-terminus of the wild-type NiV-G protein (SEQ ID NO:138), 39 contiguous amino acid residues at or near the N-terminus of the wild-type NiV-G protein (SEQ ID NO:138), or 40 contiguous amino acid residues at or near the N-terminus of the wild-type NiV-G protein (SEQ ID NO:138).

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A, and E533A with reference to numbering set forth in SEQ ID NO:138.

In some embodiments, the G protein is a mutant G protein containing one or more amino acid substitutions selected from the group consisting of E501A, W504A, Q530A, and E533A with reference to numbering set forth in SEQ ID NO:138. In some embodiments, the G protein is a mutant G protein that contains one or more amino acid substitutions selected from the group consisting of E501A, W504A, Q530A, and E533A with reference to SEQ ID NO:138 or a biologically active portion thereof containing an N-terminal truncation. In some embodiments, the G protein is a mutant G protein that contains one or more amino acid substitutions selected from the group consisting of E501A. W504A, Q530A, and E533A in combination with any one of the N-terminal truncations disclosed above with reference to SEQ ID NO:138 or a biologically active portion thereof. In some embodiments, any of the mutant G proteins described above contains one, two, three, or all four amino acids selected from the group consisting of E501A, W504A, Q530A, and E533A with reference to numbering set forth in SEQ ID NO:138, in all pairwise and triple combinations thereof.

In some embodiments, the mutant NiV-G protein has the amino acid sequence set forth in SEQ ID NO: 127 or 155 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO: 127 or 155. In some embodiments, the G protein has the sequence of amino acids set forth in SEQ ID NO: 127 or 155.

In some embodiments, the targeted envelope protein contains a G protein or a functionally active variant or biologically active portion thereof and a targeting antibody or antigen binding fragment thereof, in which the targeted envelope protein exhibits increased binding for another molecule that is different from the native binding partner of a wild-type G protein. In some embodiments, the targeting antibody or antigen binding fragment thereof is a single domain antibody (sdAb) or a scFv. In some embodiments, the other molecule is a protein expressed on the surface of a desired target cell. In some embodiments, the increased binding to the other molecule is increased by greater than at or about 25%, at or about 30%, at or about 40%, at or about 50%, at or about 60%, at or about 70%, at or about 80%, at or about 90%, or at or about 100%. In some embodiments, the binding confers re-targeted binding compared to the binding of a wild-type G protein in which a new or different binding activity is conferred.

In some embodiments, the C-terminus of the targeting antibody or antigen binding fragment thereof is attached to the C-terminus of the G protein or biologically active portion thereof. In some embodiments, the N-terminus end of the targeting antibody or antigen binding fragment thereof is exposed on the exterior surface of the lipid bilayer. In some embodiments, the N-terminus end of the targeting antibody or antigen binding fragment thereof binds to a cell surface molecule of a target cell. In some embodiments, the targeting antibody or antigen binding fragment thereof specifically binds to a cell surface molecule present on a target cell. In some embodiments, the cell surface molecule is a protein, glycan, lipid, or low molecular weight molecule.

In some embodiments, the cell surface molecule of a target cell is an antigen or portion thereof. In some embodiments, the targeting antibody or antigen binding fragment thereof is an antibody having a single monomeric domain antigen binding/recognition domain that is able to bind selectively to a specific antigen. In some embodiments, the single domain antibody binds an antigen present on a target cell.

Exemplary cells include immune effector cells, peripheral blood mononuclear cells (PBMC) such as lymphocytes (T cells. B cells, natural killer cells) and monocytes, granulocytes (neutrophils, basophils, eosinophils), macrophages, dendritic cells, cytotoxic T lymphocytes, polymorphonuclear cells (also known as PMN, PML, or PMNL), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes.

In some embodiments, the target cell is a cell of a target tissue. In some embodiments, the target tissue is liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g., hepatocyte), or a cardiac cell (e.g., cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g., a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+B cell, a CD19+B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacytoid dendritic cell, a CD11c+ cell, a CD11b+ cell, a splenocyte, a B cell, a hepatocyte, an endothelial cell, or a non-cancerous cell. In some embodiments, the cell surface molecule is any one of CD8.

In some embodiments, the G protein or functionally active variant or biologically active portion thereof is linked directly to the sdAb variable domain (e.g., a VHH) or scFv. In some embodiments, the targeted envelope protein is a fusion protein that has the following structure: (N′-single domain antibody-C′)-(C′-G protein-N′). In some embodiments, the targeted envelope protein is a fusion protein that has the following structure: (N′-scFv-C′)-(C′-G protein-N′).

In some embodiments, the G protein or functionally active variant or biologically active portion thereof is linked indirectly via a linker to the sdAb variable domain or scFv. In some embodiments, the linker is a peptide linker. In some embodiments, the linker is a chemical linker.

In some embodiments, the linker is a peptide linker and the targeted envelope protein is a fusion protein containing the G protein or functionally active variant or biologically active portion thereof linked via a peptide linker to the sdAb variable domain or scFv. In some embodiments, the targeted envelope protein is a fusion protein that has the following structure: (N′-single domain antibody-C′)-Linker-(C′-G protein-N′). In some embodiments, the targeted envelope protein is a fusion protein that has the following structure: (N′-scFv-C′)-Linker-(C′-G protein-N′). In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids. 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids. 14 to 36 amino acids, 14 to 32 amino acids. 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids. 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 38 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In some embodiments, the linker is a flexible peptide linker. In some such embodiments, the linker is 1-20 amino acids, such as 1-20 amino acids comprising glycine. In some embodiments, the linker is 1-20 amino acids, such as 1-20 amino acids comprising glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO:147), GGGGGS (SEQ ID NO:145) or combinations thereof. In some embodiments, the polypeptide linker has the sequence (GGS)n, (SEQ ID NO:231) wherein n is 1 to 10. In some embodiments, the polypeptide linker has the sequence (GGGGS)n, (SEQ ID NO:146) wherein n is 1 to 10. In some embodiments, the polypeptide linker has the sequence (GGGGGS)n (SEQ ID NO:137), wherein n is 1 to 6.

Also provided herein are polynucleotides comprising a nucleic acid sequence encoding a targeted envelope protein. In some embodiments, the polynucleotides comprise a nucleic acid sequence encoding a G protein or biologically active portion thereof. In some embodiments, the polynucleotides further comprise a nucleic acid sequence encoding a single domain antibody (sdAb) variable domain or scFv or biologically active portion thereof. The polynucleotides may include a sequence of nucleotides encoding any of the targeted envelope proteins described above. In some embodiments, the polynucleotide is a synthetic nucleic acid. Also provided are expression vectors containing any of the provided polynucleotides.

In some embodiments, expression of natural or synthetic nucleic acids is achieved by operably linking a nucleic acid encoding the gene of interest to a promoter and incorporating the construct into an expression vector. In some embodiments, vectors are suitable for replication and integration in eukaryotes. In some embodiments, cloning vectors contain transcription and translation terminators, initiation sequences, and promoters useful for expression of the desired nucleic acid sequence. In some of any embodiments, a plasmid comprises a promoter suitable for expression in a cell.

In some embodiments, the polynucleotides contain at least one promoter that is operatively linked to control expression of the targeted envelope protein containing the G protein and the single domain antibody (sdAb) variable domain or scFv. For expression of the targeted envelope protein, at least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

In some embodiments, additional promoter elements, e.g., enhancers, regulate the frequency of transcriptional initiation. In some embodiments, additional promoter elements are located in the region 30-110 bp upstream of the start site, although a number of promoters have been shown to contain functional elements downstream of the start site as well. In some embodiments, spacing between promoter elements frequently is flexible, so that promoter function is preserved when elements are inverted or moved relative to one another. In some embodiments, such as with the thymidine kinase (tk) promoter, the spacing between promoter elements is increased to 50 bp apart before activity begins to decline. In some embodiments, depending on the promoter, individual elements can function either cooperatively or independently to activate transcription.

In some embodiments, a promoter is one naturally associated with a gene or polynucleotide sequence, as is obtained by isolating the 5′ non-coding sequences located upstream of the coding segment and/or exon. In some embodiments, such a promoter is referred to as “endogenous.” In some embodiments, an enhancer is one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell. and promoters or enhancers not “naturally occurring,” i.e., containing different elements of different transcriptional regulatory regions, and/or mutations that alter expression. In addition to producing nucleic acid sequences of promoters and enhancers synthetically, sequences are produced using recombinant cloning and/or nucleic acid amplification technology. including PCR, in connection with the compositions disclosed herein.

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor-Ia (EF-Ia). In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In some embodiments, the promoter is an inducible promoter. In some embodiments, the inducible promoter provides a molecular switch capable of turning on expression of the polynucleotide sequence to which it is operatively linked when such expression is desired, or turning off the expression when expression is not desired. In some embodiments, inducible promoters comprise a metallothionine promoter, a glucocorticoid promoter, a progesterone promoter, and a tetracycline promoter.

In some embodiments, exogenously controlled inducible promoters are used to regulate expression of the G protein and single domain antibody (sdAb) variable domain or scFv. For example, radiation-inducible promoters, heat-inducible promoters, and/or drug-inducible promoters are used to selectively drive transgene to expression in, for example, targeted regions. In such embodiments, the location, duration, and level of transgene expression are regulated by the administration of the exogenous source of induction.

In some embodiments, expression of the targeted envelope protein containing a G protein and single domain antibody (sdAb) variable domain or scFv is regulated using a drug-inducible promoter. For example, in some embodiments, the promoter, enhancer, or transactivator comprises a Lac operator sequence, a tetracycline operator sequence, a galactose operator sequence, a doxycycline operator sequence, a rapamycin operator sequence, a tamoxifen operator sequence, or a hormone-responsive operator sequence, or an analog thereof. In some instances, the inducible promoter comprises a tetracycline response element (TRE). In some embodiments, the inducible promoter comprises an estrogen response element (ERE), which can activate gene expression in the presence of tamoxifen. In some instances, a drug-inducible element, such as a TRE, is combined with a selected promoter to enhance transcription in the presence of drug, such as doxycycline. In some embodiments, the drug-inducible promoter is a small molecule-inducible promoter.

In some embodiments, any of the provided polynucleotides are modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In order to assess the expression of the targeted envelope protein, the expression vector to be introduced into a cell can also contain either a selectable marker gene or a reporter gene or both to facilitate identification and selection of expressing particles, e.g., viral particles. In other embodiments, the selectable marker is carried wo on a separate piece of DNA and used in a co-transfection procedure. In some embodiments, both selectable markers and reporter genes are flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000, FEBS Lett. 479:79-82). Suitable expression systems are well known and may be prepared using well known techniques or obtained commercially. In some embodiments, internal deletion constructs are generated using unique internal restriction sites or by partial digestion of non-unique restriction sites. Constructs may then be transfected into cells that display high levels of the desired polynucleotide and/or polypeptide expression. In general, the construct with the minimal 5′flanking region showing the highest level of expression of reporter gene is identified as the promoter. In some embodiments, such promoter regions are linked to a reporter gene and used to evaluate agents for the ability to modulate promoter-driven transcription.

i. Mutated Paramyxovirus G Protein

In some embodiments, the paramyxovirus G proteins are mutant Paramyxovirus G glycoproteins (e.g., variant Paramyxovirus G glycoproteins) comprising one or more amino acid mutations (i.e., substitutions) that result in decreased glycosylation of the protein. The one or more amino acid mutations, also called deglycosylation mutations, can be one or more amino acid substitutions (also referred to as mutations).

In some embodiments, the mutant Paramyxovirus G glycoprotein comprises an amino acid substitution at one or more amino acid positions that reduce glycosylation of the G glycoprotein. In some embodiments, the one or more amino acid substitutions disrupts an N-linked glycosylation site. In some embodiments, the one or more amino acid substitutions disrupts an O-linked glycosylation site.

In some embodiments, the mutant Paramyxovirus G glycoprotein is derived from Morbillivirus (e.g., measles virus (MeV), canine distemper virus, Cetacean morbillivirus, Peste-des-petits-ruminants virus, Phocine distemper virus, Rinderpest virus), Henipavirus (e.g., Hendra (HeV) virus, Nipah (NiV) virus, a Cedar (CedPV) virus, Mòjiãng virus, a Langya virus or bat Paramyxovirus). In some embodiments, the mutant Paramyxovirus G glycoprotein is a mutant of a Paramyxovirus G glycoprotein derived from Nipah virus or Measles virus. In some embodiments, the mutant Paramyxovirus G protein is a mutant of a Paramyxovirus G protein selected from the group consisting of SEQ ID NOs:127, 138, and 155, or a modified Paramyxovirus G glycoprotein derived from any one of SEQ ID NO:127, 138, and 155 containing an altered cytoplasmic tail. In some embodiments, the mutant Paramyxovirus G protein has a sequence of amino acids that has at least 85%, at least 88%, at least 87%, at least 88%, at least 89%, at least 90%, at least 91%, at least 92%, at least 93%, at least 94% or at least 95% to any one of SEQ ID NOs:127, 138, and 155 and contains the acid substitution at one or more amino acid positions that reduce glycosylation of the G glycoprotein as provided herein.

The location of precited glycosylation sites can be determined using the sequence of a protein. For example, N-glycosylation often occurs at sites with the sequence N—X-S/T in which X is any amino acid except P. Various algorithms and tools are available for prediction of both N- and O-linked glycosylation, including SprintGly (http://sparks-lab.org/server/sprint-gly/), NetNGlyc (https://services.healthtech.dtu.dk/service.php?NetNGlyc-1.0), NetOGlyc (https://services.healthtech.dtu.dk/service.php?NetOGlyc-4.0), and GlycoMinestruct (http//glycomine.erc.monash.edu/Lab/GlycoMine_Struct/), and methods described in Pitti et al., Sci. Reports. 9:15975 (2019) and Pakhrin et al., Molecules 26:7314 (2021). Any predicted glycosylation site may be substituted as described herein.

In some embodiments, the Paramyxovirus G glycoprotein to which the deglycosylation mutation is made is a NiV-G set forth in SEQ ID NO:138 or a modified Nipah G glycoprotein (NiV-G) that has an altered cytoplasmic tail compared to native NiV-G (e.g., SEQ ID NO:138). In some embodiments, the variant Paramyxovirus G protein has a sequence of amino acids that has at least 85%, at least 88%, at least 87%, at least 88%, at least 89%, at least 90%, at least 91%, at least 92%, at least 93%, at least 94% or at least 95% to SEQ ID NO:138 and contains the acid substitution at one or more amino acid positions that reduce glycosylation of the G glycoprotein as provided herein. In some embodiments, the Paramyxovirus G glycoprotein to which the deglycosylation mutation is made is a NiV-G set forth in SEQ ID NO:127 or a modified Nipah G glycoprotein (NiV-G) that has an altered cytoplasmic tail compared to native NiV-G (e.g., SEQ ID NO:127). In some embodiments, the variant Paramyxovirus G protein has a sequence of amino acids that has at least 85%, at least 86%, at least 87%, at least 88%, at least 89%, at least 90%, at least 91%, at least 92%, at least 93%, at least 94% or at least 95% to SEQ ID NO:127 and contains the acid substitution at one or more amino acid positions that reduce glycosylation of the G glycoprotein as provided herein.

Exemplary modified NiV-G proteins with altered cytoplasmic tails to which the one or more amino acid substitutions for reducing glycosylation can be incorporated are as described herein, see, for example, Table 26.

Amino acid positions for substitutions are described herein with positions “corresponding to” positions of a reference sequence. It is understood that the amino acid substitutions are not limited to being made in only the reference sequence but also can be made in similar sequences by identification of residues that align or correspond with the reference positions. For instance, positions “corresponding to” to positions of a protein in a reference sequence can be identified upon alignment of a similar sequence with the referenced sequence based on structural sequence alignment or using a standard alignment algorithm, such as the GAP algorithm. By aligning the sequences, one skilled in the art can identify corresponding residues, for example, using conserved and identical amino acid residues as guides. For instance, amino acid positions for mutations are described herein with reference to the exemplary truncated NiV-G sequence set forth in SEQ ID NO:127; however, similar amino acid positions for mutations as described can be made in other modified NiV-G sequences, such as any as described herein, see, for example, Table 26, by sequence alignment and identification of the corresponding residues.

In some embodiments, the one or more amino acid mutations are at positions corresponding to positions 39, 126, 128, 273, 345, 384, 448, and 496 of SEQ ID NO:138. In some embodiments, the one or more amino acid mutations are at positions corresponding to positions 39, 126, 128, 273, 345, 384, 448, and 496 of SEQ ID NO:138, and where SEQ ID NO:138 also includes one or more mutations selected from: E501 A, W504A, Q530A, and E533A. In some embodiments, the variant Paramyxovirus G glycoprotein comprises an amino acid mutation at any one of positions 39, 126, 128, 273, 345, 384, 448, and 496 of SEQ ID NO:138. In some embodiments, the variant Paramyxovirus G glycoprotein comprises an amino acid mutation at any one of positions 39, 126, 128, 273, 345, 384, 448, and 496 of SEQ ID NO:138, and where SEQ ID NO:138 also includes one or more mutations selected from: E501 A, W504A, Q530A, and E533A. In some embodiments, the variant Paramyxovirus G glycoprotein comprises two or more amino acid mutations at any of positions corresponding to positions 39, 126, 128, 273, 345, 384, 448, and 496 of SEQ ID NO:138, such as mutations at 2, 3, 4, 5, 7, or 8 of the positions. In some embodiments, the variant Paramyxovirus G glycoprotein comprises two or more amino acid mutations at any of positions corresponding to positions 39, 126, 128, 273, 345, 384, 448, and 496 of SEQ ID NO:138, such as mutations at 2, 3, 4, 5, 7, or 8 of the positions, and where SEQ ID NO:138 also includes one or more mutations selected from: E501 A, W504A, Q530A, and E533A.

In some embodiments, the one or more amino acid mutations is at a position corresponding to position 39 of SEQ ID NO:138. In some embodiments, the one or more amino acid mutations is at a position corresponding to position 126 of SEQ ID NO:138. In some embodiments, the one or more amino acid mutations is at a position corresponding to position 128 of SEQ ID NO:138. In some embodiments, the one or more amino acid mutations is at a position corresponding to position 273 of SEQ ID NO:138. In some embodiments, the one or more amino acid mutations is at a position corresponding to position 345 of SEQ ID NO:138. In some embodiments, the one or more amino acid mutations is at a position corresponding to position 384 of SEQ ID NO:138. In some embodiments, the one or more amino acid mutations is at a position corresponding to position 448 of SEQ ID NO:138. In some embodiments, the one or more amino acid mutations is at a position corresponding to position 496 of SEQ ID NO:138. In such embodiments, SEQ ID NO:138 can also include one or more mutations selected from: E501 A, W504A, QS30A, and E533A.

In some embodiments, the native amino acid at the position comprising the amino acid mutation is asparagine or serine. In some embodiments, the amino acid mutation is an amino acid substitution. In some embodiments, the mutation is an asparagine to glutamine substitution, In some embodiments, the mutation is a serine to alanine substitution.

In some embodiments, the mutation is an asparagine to glutamine substitution at a position corresponding to position 39 (N39Q) of SEQ ID NO:138. In some embodiments, the mutation is an asparagine to glutamine substitution at a position corresponding to position 126 (N126Q) of SEQ ID NO:138. In some embodiments, the mutation is an asparagine to glutamine substitution at a position corresponding to position 273 (N273Q) of SEQ ID NO:138. In some embodiments, the mutation is an asparagine to glutamine substitution at a position corresponding to position 345 (N345Q) of SEQ ID NO:138. In some embodiments, the mutation is an asparagine to glutamine substitution at a position corresponding to position 384 (N384Q) of SEQ ID NO:138. In some embodiments, the mutation is an asparagine to glutamine substitution at a position corresponding to position 448 (N448Q) of SEQ ID NO:138. In some embodiments, the mutation is an asparagine to glutamine substitution at a position corresponding to position 496 (N4960) of SEQ ID NO:138. In such embodiments, SEQ ID NO:138 can also include one or more mutations selected from: E501 A, W504A, Q530A, and E533A.

In some embodiments, the mutation is a serine to alanine substitution at a position corresponding to position 128 (S128A) of SEQ ID NO:138. In such embodiments, SEQ ID NO:138 can also include one or more mutations selected from: E501 A, W504A, Q530A, and E533A.

In some embodiments, the G glycoprotein is derived from Nipah virus G protein and the one or more amino acid substitutions are at positions corresponding to positions selected from the group consisting of 39, 126, 128, 273, 345, 384, 448, and 496 of SEQ ID NO:138, and where SEQ ID NO:138 can also include one or more mutations selected from: E501 A, W504A, Q530A, and E533A. In some embodiments, the one or more amino acid substitutions are selected from N39Q, N1260, S128A, N273Q, N345Q, N3840, N448Q, N4960 or any combination thereof. In some embodiments, the G glycoprotein is a mutant NiV-G containing one amino acid substitution from any one of N39Q, N1260, S128A, N273Q, N345Q, N384Q, N448Q, N496Q. In some embodiments, the G glycoprotein is a mutant NiV-G containing two amino acid substitutions from any two of N39Q, N1260, S128A, N273Q, N345Q, N384Q, N448Q, N496Q. In some embodiments, the G glycoprotein is a mutant NiV-G containing three amino acid substitutions from any three of N39Q, N126Q, S128A, N273Q, N345Q, N384Q, N4480, N4960. In some embodiments, the G glycoprotein is a mutant NiV-G containing four amino acid substitutions from any one of N39Q, N126Q, S128A, N273Q, N345Q. N384Q, N448Q, N4960. In some embodiments, the G glycoprotein is a mutant NiV-G containing five amino acid substitutions from any one of N39Q, N126Q, S128A, N273Q, N345Q, N384Q, N448Q, N496Q. In some embodiments, the G glycoprotein is a mutant NiV-G containing six amino acid substitutions from any one of N39Q, N126Q, S128A, N273Q, N345Q, N384Q, N4480, N496Q. In some embodiments, the G glycoprotein is a mutant NiV-G containing seven amino acid substitutions from any one of N39Q, N126Q. S128A. N273Q, N345Q, N384Q, N448Q, N496Q. In some embodiments, the G glycoprotein is a mutant NiV-G containing eight amino acid substitutions from any one of N390, N126Q, S128A, N273Q, N345Q, N384Q, N448Q, N496Q. In some embodiments, the one or more amino acid substitutions are in the SEQ ID NO:138 or a or a modified Nipah G glycoprotein (NiV-G) that has an altered cytoplasmic tail compared to native NiV-G (e.g., SEQ ID NO:138). In some embodiments, the amino acid substitutions are in a modified NiV-G protein described herein, see, for example, Table 26. In some embodiments, the amino acid substitutions are in the NiV-G set forth in SEQ ID NO:138. In such embodiments, SEQ ID NO:138 can also include one or more mutations selected from: E501 A, W504A, Q530A, and E533A.

In some embodiments, the variant Nipah-G protein comprises at least three amino acid substitutions. In some embodiments, the amino acid substitutions are at positions 273, 384, and 496 of SEQ ID NO:138. In some embodiments, the amino acid substitutions are at positions 273, 345, and 496 of SEQ ID NO:138. In some embodiments, the amino acid substitutions are at positions 39, 126, and 128 of SEQ ID NO:138. In some embodiments, the amino acid substitutions are at positions 39, 273, and 345 of SEQ ID NO:138. In some embodiments, the amino acid substitutions are at positions 39, 384, and 448 of SEQ ID NO:138. In some embodiments, the amino acid substitutions are at positions 39, 448, and 496 of SEQ ID NO:138. In some embodiments, the amino acid substitutions are at positions 39, 128, and 273 of SEQ ID NO:138. In some embodiments, the amino acid substitutions are at positions 39, 345, and 384 of SEQ ID NO:138. In some embodiments, the amino acid substitutions are at positions 39, 384, and 448 of SEQ ID NO:138. In such embodiments, SEQ ID NO: 138 can also include one or more mutations selected from: E501 A, W504A, Q530A, and E533A.

In some embodiments, the variant Nipah-G protein comprises at least two amino acid substitutions. In some embodiments, the amino acid substitutions are at positions 273, and 496 of SEQ ID NO:138. In some embodiments, the amino acid substitutions are at positions 345, and 496 of SEQ ID NO:138. In some embodiments, the amino acid substitutions are at positions 39 and 128 of SEQ ID NO:138. In some embodiments, the amino acid substitutions are at positions 39, and 345 of SEQ ID NO: 138. In some embodiments, the amino acid substitutions are at positions 39, and 448 of SEQ ID NO:138. In some embodiments, the amino acid substitutions are at positions 39 and 496 of SEQ ID NO:138. In some embodiments, the amino acid substitutions are at positions 39 and 273 of SEQ ID NO:138. In some embodiments, the amino acid substitutions are at positions 39 and 384 of SEQ ID NO:138. In some embodiments, the amino acid substitutions are at positions 384 and 448 of SEQ ID NO:138. In such embodiments, SEQ ID NO: 138 can also include one or more mutations selected from: E501 A, W504A, Q530A, and E533A.

In some embodiments, the amino acid substitution is at position 39 of SEQ ID NO:138. In some embodiments, the amino acid substitution is at position 126 of SEQ ID NO:138. In some embodiments, the amino acid substitution is at position 128 of to SEQ ID NO:138. In some embodiments, the amino acid substitution is at position 273 of SEQ ID NO:138. In some embodiments, the amino acid substitution is at position 345 of SEQ ID NO:138. In some embodiments, the amino acid substitution is at position 384 of SEQ ID NO:138. In some embodiments, the amino acid substitution is at position 448 of SEQ ID NO:138. In some embodiments, the amino acid substitution is at position 496 of SEQ ID NO:138. In such embodiments, SEQ ID NO:138 can also include one or more mutations selected from: E501 A, W504A, Q530A, and E533A.

In some embodiments, the mutant Nipah-G protein comprises an asparagine to glutamine substitution at position 39 of SEQ ID NO:138. In some embodiments, the mutant Nipah-G protein comprises an asparagine to glutamine substitution at position 126 of SEQ ID NO:138. In some embodiments, the mutant Nipah-G protein comprises an asparagine to glutamine substitution at position 273 of SEQ ID NO:138. In some embodiments, the mutant Nipah-G protein comprises an asparagine to glutamine substitution at position 345 of SEQ ID NO:138. In some embodiments, the mutant Nipah-G protein comprises an asparagine to glutamine substitution at position 384 of SEQ ID NO:138. In some embodiments, the mutant Nipah-G protein comprises an asparagine to glutamine substitution at position 448 of SEQ ID NO:138. In some embodiments, the mutant Nipah-G protein comprises an asparagine to glutamine substitution at position 496 of SEQ ID NO:138. In some embodiments, the mutant Nipah-G protein comprises a serine to alanine substitution at position 128 of SEQ ID NO:138. In such embodiments, SEQ ID NO:138 can also include one or more mutations selected from: E501 A, W504A, Q530A, and E533A. In some embodiments, the mutant Nipah-G protein comprises an asparagine to glutamine substitution at position 273 of SEQ ID NO: 138, and SEQ ID NO: 138 includes one or more mutations selected from: E501 A, W504A, Q530A, and E533A.

### E. F Protein

In some embodiments, the targeted lipid particle comprises one or more fusogens, e.g., henipavirus F proteins. In some embodiments, the targeted lipid particle contains an exogenous or overexpressed fusogen. In some embodiments, the fusogen is disposed in the lipid bilayer. In some embodiments, the fusogen facilitates the fusion of the targeted particle's lipid bilayer to a membrane. In some embodiments, the membrane is a plasma cell membrane.

In some embodiments, fusogens comprise protein based, lipid based, and chemical based fusogens. In some embodiments, the targeted lipid particle comprises a first fusogen comprising a protein fusogen and a second fusogen comprising a lipid fusogen or chemical fusogen. In some embodiments, the fusogen binds a fusogen binding partner on a target cell surface.

In some embodiments, the fusogen comprises a protein with a hydrophobic fusion peptide domain. In some embodiments, the fusogen comprises a henipavirus F protein molecule or biologically active portion thereof. In some embodiments, the Henipavirus F protein is a Hendra (Hev) virus F protein, a Nipah (NiV) virus F-protein, a Cedar (CedPV) virus F protein, a Mojiang virus F protein, a bat Paramyxovirus F protein, or a biologically active portion thereof. Table 26 provides a list of non-limiting examples of F proteins.

In some embodiments, the N-terminal hydrophobic fusion peptide domain of the F protein molecule or biologically active portion thereof is exposed on the outside of a lipid bilayer.

F proteins of henipaviruses are encoded as F0 precursors containing a signal peptide (e.g., corresponding to amino acid residues 1-26 of SEQ ID NO: 110). Following cleavage of the signal peptide, the mature F0 (e.g., gSEQ ID NO: 111) is transported to the cell surface, then endocytosed and cleaved by cathepsin L (e.g., between amino acids 109-110 of SEQ ID NO: 110) into the mature fusogenic subunits F1 (e.g., corresponding to amino acids 110-546 of SEQ ID NO:110; set forth in SEQ ID NO:113) and F2 (e.g., corresponding to amino acid residues 27-109 of SEQ ID NO:110; set forth in SEQ ID NO:112). The F1 and F2 subunits are associated by a disulfide bond and recycled back to the cell surface. The F1 subunit contains the fusion peptide domain located at the N terminus of the F1 subunit (e.g., corresponding to amino acids 110-129 of SEQ ID NO:110) where it is able to insert into a cell membrane to drive fusion. In some embodiments, fusion activity is blocked by association of the F protein with G protein, until G engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some embodiments, the henipavirus F proteins exhibit compatibility with G proteins from other species to trigger fusion (Brandel-Tretheway et al. Journal of Virology. 2019. 93(13):e00577-19). In some aspects of the provided targeted lipid particle, the F protein is heterologous to the G protein, i.e., the F and G protein or biologically active portions thereof are from different henipavirus species. For example, the F protein Is from Hendra virus and the G protein is from Nipah virus. In other aspects, the F protein is a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some embodiments, the chimeric F protein contains an extracellular domain from one henipavirus species and a transmembrane and/or cytoplasmic domain from a different henipavirus species. For example, the F protein may contain an extracellular domain of Hendra virus and a transmembrane/cytoplasmic domain of Nipah virus. F protein sequences disclosed herein are predominantly disclosed as expressed sequences including an N-terminal signal sequence. Such N-terminal signal sequences are commonly cleaved co- or post-translationally, thus the mature protein sequences for all F protein sequences disclosed herein are also contemplated as lacking the N-terminal signal sequence.

In some embodiments, the F protein is encoded by a nucleotide sequence that encodes the sequence set forth by any one of SEQ ID NOs: 110, 111, 128, 134-138, or 161-164, or is a functionally active variant or a biologically active portion thereof that has a sequence that is at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to anyone of SEQ ID NOs: 110, 111, 128, 134-136, or 161-164. In some embodiments, the F protein or the functionally active variant or biologically active portion thereof retains fusogenic activity in conjunction with a Henipavirus G protein, such as a G protein set forth herein. Fusogenic activity includes the activity of the F protein in conjunction with a Henipavirus G protein to promote or facilitate fusion of two membrane lumens, such as the lumen of the targeted lipid particle having embedded in its lipid bilayer a henipavirus F and G protein, and a cytoplasm of a target cell, e.g., a cell that contains a surface receptor or molecule that is recognized or bound by the targeted envelope protein. In some embodiments, the F protein and G protein are from the same Henipavirus species (e.g., NiV-G and NiV-F). In some embodiments, the F protein and G protein are from different Henipavirus species (e.g., NiV-G and HeV-F). In some embodiments, the F protein of the functionally active variant or biologically active portion retains the cleavage site cleaved by cathepsin L (e.g., corresponding to the cleavage site between amino acids 109-110 of SEQ ID NO:110).

In some embodiments, the F protein has the sequence of amino acids set forth in SEQ ID NO:110, SEQ ID NO:111, SEQ ID NO:128, SEQ ID NO:134, SEQ ID NO:135, SEQ ID NO:136, SEQ ID NO:161, SEQ ID NO:162, SEQ ID NO:163, or SEQ ID NO:164 or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:110, SEQ ID NO:111, SEQ ID NO:128, SEQ ID NO:134, SEQ ID NO:135, SEQ ID NO:136, SEQ ID NO:161, SEQ ID NO:162, SEQ ID NO:163, or SEQ ID NO:164 and retains fusogenic activity in conjunction with a Henipavirus G protein (e.g., NiV-G or HeV-G). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:110, SEQ ID NO:111, SEQ ID NO:128, SEQ ID NO:134, SEQ ID NO:135, SEQ ID NO:136, SEQ ID NO:161, SEQ ID NO:162, SEQ ID NO:163, or SEQ ID NO:164 and retains fusogenic activity in conjunction with a Henipavirus G protein (e.g., NiV-G or HeV-G).

Reference to retaining fusogenic activity includes activity (in conjunction with a Henipavirus G protein) that is at or about 10% to at or about 150% or more of the level or degree of binding of the corresponding wild-type F protein, such as set forth in SEQ ID NO:110, SEQ ID NO:111, SEQ ID NO:128, SEQ ID NO:134, SEQ ID NO:135, SEQ ID NO:136, SEQ ID NO:161, SEQ ID NO:162, SEQ ID NO:163, or SEQ ID NO:164, such as at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type f protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions, or truncations. In some embodiments, the mutations described herein relate to amino acid insertions, deletions, substitutions, or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein, or a bat Paramyxovirus F protein. In some embodiments, the wild-type F protein is encoded by a sequence of nucleotides that encodes any one of SEQ ID NO: 110, 111, 128, 134-136, or 161-164.

In some embodiments, the mutant F protein is a biologically active portion of a wild-type F protein that is an N-terminally and/or C-terminally truncated fragment. In some embodiments, the mutant F protein or the biologically active portion of a wild-type F protein thereof comprises one or more amino acid substitutions. In some embodiments, the mutations described herein can improve transduction efficiency. In some embodiments, the mutations described herein can increase fusogenic capacity. Exemplary mutations include any as described, see e.g., Khetawat and Broder 2010 Virology Journal 7:312; Witting et al. 2013 Gene Therapy 20:997-1005; published international; patent application No. WO/20131148327.

In some embodiments, the mutant F protein is a biologically active portion that is truncated and lacks up to 20 contiguous amino acid residues at or near the C-terminus of the wild-type F protein, such as a wild-type F protein encoded by a sequence of nucleotides encoding the F protein set forth in any one of SEQ ID NOs: 110, 111, 128. or 134-136. In some embodiments, the mutant F protein is truncated and lacks up to 19 contiguous amino acids, such as up to 18, 17, 16, 15, 14, 13, 12, 11, 10, 9, 8, 7, 6, 5, 4, 3, 2, or 1 contiguous amino acid(s) at the C-terminus of the wild-type F protein.

In some embodiments, the F protein or the functionally active variant or biologically active portion thereof comprises an F1 subunit or a fusogenic portion thereof. In some embodiments, the F1 subunit is a proteolytically cleaved portion of the F0 precursor. In some embodiments, the F0 precursor is inactive. In some embodiments, the cleavage of the F0 precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

In some embodiments, the F protein is a wild-type Nipah virus F (NiV-F) protein or is a functionally active variant or biologically active portion thereof. In some embodiments, the F0 precursor is encoded by a sequence of nucleotides encoding the sequence set forth in SEQ ID NO:110. The encoding nucleic acid can encode a signal peptide sequence that has the sequence MVVILDKRCY CNLLILILMI SECSVG (SEQ ID NO:144) or another signal peptide sequence. In some embodiments, the F protein has the sequence set forth in SEQ ID NO:111. In some examples, the F protein is cleaved into an F1 subunit comprising the sequence set forth in SEQ ID NO:113 and an F2 subunit comprising the sequence set forth in SEQ ID NO:112.

In some embodiments, the F protein is a NiV-F protein that is encoded by a sequence of nucleotides encoding the sequence set forth in SEQ ID NO:110, or is a functionally active variant or biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:110. In some embodiments, the NiV-F-protein has the sequence of set forth in SEQ ID NO:111, or is a functionally active variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:111. In some embodiments, the F protein or the functionally active variant or biologically active portion thereof retains the cleavage site cleaved by cathepsin L (e.g., corresponding to the cleavage site between amino acids 109-110 of SEQ ID NO:110).

In some embodiments, the F protein or the functionally active variant or the biologically active portion thereof includes an F1 subunit that has the sequence set forth in SEQ ID NO:113, or an amino acid sequence having, at least at or about 80%, at least at or about 81%, at least at or about 82%, at least at or about 83%, at least at or about 84%, at least at or about 85%, at least at or about 86%, at least at or about 87%, at least at or about 88%, at least at or about 89% at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEO ID NO:113.

In some embodiments, the F protein or the functionally active variant or biologically active portion thereof includes an F2 subunit that has the sequence set forth in SEQ ID NO:112, or an amino acid sequence having, at least at or about 80%, at least at or about 81%, at least at or about 82%, at least at or about 83%, at least at or about 84%, at least at or about 85%, at least at or about 88%, at least at or about 87%, at least at or about 88%, at least at or about 89% at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:112.

In some embodiments, the F protein is a mutant NiV-F protein that is a biologically active portion thereof that is truncated and lacks up to 20 contiguous amino acid residues at or near the C-terminus of the wild-type NiV-F protein (e.g., set forth SEQ ID NO:111). In some embodiments, the mutant NiV-F protein comprises an amino acid sequence set forth in SEQ ID NO:114. In some embodiments, the mutant NiV-F protein has a sequence that has at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:114. In some embodiments, the mutant F protein contains an F1 protein that has the sequence set forth in SEQ ID NO:115. In some embodiments, the mutant F protein has a sequence that has at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:115.

In some embodiments, the F protein is a mutant NiV-F protein that is a biologically active portion thereof that comprises a 20 amino acid truncation at or near the C-terminus of the wild-type NW-F protein (SEQ ID NO:111); and a point mutation on an N-linked glycosylation site. In some embodiments, the mutant NW-F protein comprises an amino acid sequence set forth in SEQ ID NO:116. In some embodiments, the mutant NW-F protein has a sequence that has at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:116.

In some embodiments, the F protein is a mutant NV-F protein that is a biologically active portion thereof that comprises a 22 amino acid truncation at or near the C-terminus of the wild-type NiV-F protein (SEQ ID NO:111). In some embodiments, the NiV-F protein comprises an amino acid sequence set forth in SEQ ID NO:117. In some embodiments, the NiV-F protein has a sequence with at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:117. In some embodiments, the NiV-F protein comprises an amino acid sequence set forth in SEQ ID NO:118. In some embodiments, the NiV-F protein has a sequence with at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:118. In some embodiments, the NiV-F protein comprises an amino acid sequence set forth in SEQ ID NO:119. In some embodiments, the NiV-F protein has a sequence with at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:119. In some embodiments, the variant F protein is a mutant Niv-F protein that has the sequence of amino acids set forth in SEQ ID NO:133. In some embodiments, the NV-F protein has a sequence with at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:133.

Methods of Generating Targeted Lipid Particles Derived from Virus

Provided herein are targeted lipid particles that are derived from virus, such as viral particles or virus-like particles, including those derived from retroviruses or lentiviruses. In some embodiments, the targeted lipid particle's bilayer of amphipathic lipids is or comprises the viral envelope. In some embodiments, the targeted lipid particle's bilayer of amphipathic lipids is or comprises lipids derived from a producer cell. In some embodiments, the viral envelope may comprise a fusogen, e.g., a fusogen that is endogenous to the virus or a pseudotyped fusogen. In some embodiments, the targeted lipid particles' lumen or cavity comprises a viral nucleic acid, e.g., a retroviral nucleic acid, e.g., a lentiviral nucleic acid. In some embodiments, the viral nucleic acid is a viral genome. In some embodiments, the targeted lipid particle further comprises one or more viral non-structural proteins, e.g., in its cavity or lumen. In some embodiments, the targeted lipid particles is or comprises a virus-like particle (VLP). In some embodiments, the VLP does not comprise an envelope. In some embodiments, the VLP comprises an envelope.

In some embodiments, the viral particle or virus-like particle, such as a retrovirus or retrovirus-like particle, comprises one or more of a Gag polyprotein, polymerase (e.g., Poi), integrase (IN, e.g., a functional or non-functional variant), protease (PR), and a fusogen. In some embodiments, the targeted lipid particle further comprises Rev. In some embodiments, one or more of the aforesaid proteins are encoded in the retroviral genome, and in some embodiments, one or more of the aforesaid proteins are provided in trans, e.g., by a helper cell, helper virus, or helper plasmid. In some embodiments, the targeted lipid particle nucleic acid (e.g., retroviral nucleic acid) comprises one or more of the following nucleic acid sequences: 5′ LTR (e.g., comprising U5 and lacking a functional U3 domain), Psi packaging element (Psi), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments the targeted lipid particle nucleic acid further comprises one or more insulator elements. In some embodiments, the recognition sites are situated between the poly A tail sequence and the WPRE.

In some embodiments, the targeted lipid particle comprises supramolecular complexes formed by viral proteins that self-assemble into capsids. In some embodiments, the targeted lipid particle is a viral particle or virus-like particle derived from viral capsids. In some embodiments, the targeted lipid particle is a viral particle or virus-like particle derived from viral nucleocapsids. In some embodiments, the targeted lipid particle comprises nucleocapsid-derived proteins that retain the property of packaging nucleic acids. In some embodiments, the viral particles or virus-like particles comprises only viral structural glycoproteins. In some embodiments, the targeted lipid particle does not contain a viral genome.

In some embodiments, the targeted lipid particle packages nucleic acids from host cells during the expression process. In some embodiments, the nucleic acids do not encode any genes involved in virus replication. In some embodiments, the targeted lipid particle is a virus-like particle, e.g., retrovirus-like particle such as a lentivirus-like particle, that is replication defective.

In some embodiments, the targeted lipid particle is a viral particle that is morphologically indistinguishable from the wild-type infectious virus. In some embodiments, the viral particle presents the entire viral proteome as an antigen. In some embodiments, the viral particle presents only a portion of the proteome as an antigen.

In some embodiments, the viral particle or virus-like particle is produced utilizing proteins (e.g., envelope proteins) from a virus within the Paramyxoviridae family. In some embodiments, the Paramyxoviridae family comprises members within the Henipavirus genus. In some embodiments, the Henipavirus is or comprises a Hendra (HeV) or a Nipah (NiV) virus. In some embodiments, the viral particles or virus-like particles incorporate a targeted envelope protein and fusogen.

In some embodiments, viral particles or virus-like particles are produced in multiple cell culture systems including bacteria, mammalian cell lines, insect cell lines, yeast, and plant cells.

Suitable cell lines which are used include, for example, CHO cells, BHK cells, MDCK cells, C3H 10T1/2 cells, FLY cells. Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells. 211 cells, 211A cells, and cyno and Macaca nemestrina cell lines. In embodiments, the packaging cells are 293 cells, 293T cells, or A549 cells.

In some embodiments, a source cell line includes a cell line which is capable of producing recombinant retroviral particles, comprising a producer cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference.

In some embodiments, the assembly of a viral particle or virus-like particle is initiated by binding of the core protein to a unique encapsidation sequence within the viral genome (e.g., UTR with stem-loop structure). In some embodiments, the interaction of the core with the encapsidation sequence facilitates oligomerization.

In some embodiments, the targeted lipid particle is a virus-like particle which comprises a sequence that is devoid of or lacking viral RNA. In some embodiments, such particles are the result of removing or eliminating the viral RNA from the sequence. In some embodiments, this is achieved by using an endogenous packaging signal binding site on Gag. In some embodiments, the endogenous packaging signal binding site is on Pol. In some embodiments, the RNA which is to be delivered will contain a cognate packaging signal. In some embodiments, a heterologous binding domain (which is heterologous to Gag) located on the RNA to be delivered, and a cognate binding site located on Gag or Pol, are used to ensure packaging of the RNA to be delivered. In some embodiments, the heterologous sequence is non-viral or it could be viral, in which case it is derived from the same virus or a different virus. In some embodiments, the vector particles could be used to deliver therapeutic RNA, in which case functional integrase and/or reverse transcriptase is not required. In some embodiments, the vector particles could also be used to deliver a therapeutic gene of interest, in which case Pot is typically included. In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a Psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. Illustrative retroviruses suitable for use in some embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV), Rous Sarcoma Virus (RSV), and other lentiviruses.

In some embodiments the retrovirus is a Gammaretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used.

In some embodiments, a vector herein is a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

In some embodiments, a viral vector comprises a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). In some embodiments, a viral vector comprises e.g., a virus or viral particle capable of transferring a nucleic acid into a cell, or the transferred nucleic acid (e.g., as naked DNA). In some embodiments, a viral vectors and transfer plasmids comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements are present in RNA form in lentiviral particles and are present in DNA form in DNA plasmids.

In some embodiments, in the vectors described herein at least part of one or more protein coding regions that contribute to or are essential for replication are absent compared to the corresponding wild-type virus. In some embodiments, the viral vector is replication-defective. In some embodiments, the vector is capable of transducing a target non-dividing host cell and/or integrating its genome into a host genome.

In some embodiments, different cells differ in their usage of particular codons. In some embodiments, this codon bias corresponds to a bias in the relative abundance of particular tRNAs in the cell type. In some embodiments, by altering the codons in the sequence so that they are tailored to match with the relative abundance of corresponding tRNAs, it is possible to increase expression. In some embodiments, it is possible to decrease expression by deliberately choosing codons for which the corresponding tRNAs are known to be rare in the particular cell type. In some embodiments, an additional degree of translational control is available. An additional description of codon optimization is found, e.g., in WO 99/41397, which is herein incorporated by reference in its entirety.

Conventional techniques for generating retrovirus vectors (and, in particular. lentivirus vectors) with or without the use of packaging/helper vectors are known to those skilled in the art and are used to generate targeted lipid particles according to the present disclosure. (See, e.g., Derse and Newbold 1993 Virology 194:530-6; Maury et al. 1994 Virology 200:632-42; Wanisch et al. 2009. Mol Ther. 1798:1316-1332; Martarano at al. 1994 J. Virol. 68:3102-11; Naldini et al., (1996a, 1996b, and 1998); Zufferey et al., 1999, J. Virol., 73:2886; Huang et al., Mol. Cell. Biol., 5:3864; Liu et al., 1995, Genes Dev., 9:1766; Cullen et al., 1991. J. Virol. 65:1053; and Cullen et al., 1991. Cell 58:423; Dull et al., 1998, U.S. Pat. Nos. 6,013,516; and 5,994,136; PCT patent applications WO 99/15683, WO 98/17815, WO 99/32646, and WO 01/79518). Conventional techniques relating to packaging vectors and producer cells known in the art may also be used according to the present disclosure. (See, e.g., Yao et al, 1998; Jones et al, 2005.)

Provided herein are targeted lipid particles that comprise a naturally derived membrane. In some embodiments, the naturally derived membrane comprises membrane vesicles prepared from cells or tissues. In some embodiments, the targeted lipid particle comprises a vesicle that is obtainable from a cell. In some embodiments, the targeted lipid particle comprises a microvesicle, an exosome, a membrane enclosed body, an apoptotic body (from apoptotic cells), a particle (which is derived from e.g., platelets), an ectosome (derivable from, e.g., neutrophiles and monocytes in serum), a prostatosome (obtainable from prostate cancer cells), or a cardiosome (derivable from cardiac cells).

In some embodiments, the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell), a precursor cell (e.g., a retinal precursor cell, a myeloblast. myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cel, a satellite cell, a radial glial cell, a bone marrow stromal cell, a pancreatic progenitor cell, an endothelial progenitor cell, a blast cell), or an immortalized ceil (e.g., HeLa, HEK293, HFF-I, MRC-5, WI-38, IMR 90, IMR 91, PER.C6, HT-1080, or BJ cell). In some embodiments, the source cell is other than a 293 cell, HEK cell, human endothelial cell, or a human epithelial cell, monocyte, macrophage, dendritic cell, or stem cell.

In some embodiments, the targeted lipid particle has a density of <1, 1-1.1, 1.05-1.15, 1.1-1.2, 1.15-1.25, 1.2-1.3, 1.25-1.35, or >1.35 g/ml. In some embodiments, the targeted lipid particle composition comprises less than 0.01%, 0.05%, 0.1%, 0.5%, 1%, 1.5%, 2%, 2.5%, 3%, 4%, 5%, or 10% source cells by protein mass, or less than 0.01%, 0.05%, 0.1%, 0.5%. 1%, 1.5%, 2%, 2.5%, 3%, 4%, 5%, or 10% of cells having a functional nucleus.

In embodiments, the targeted lipid particle has a size, or the population of targeted lipid particles have an average size, that is less than about 0.01%, 0.05%, 0.1%, 0.5%, 1%, 2%, 3%, 4%, 5%, 10%, 20%, 30%, 40%, 50%, 60%, 70%, 80%, 90%, of that of the source cell.

In some embodiments the targeted lipid particle comprises an extracellular vesicle, e.g., a cell-derived vesicle comprising a membrane that encloses an internal space and has a smaller diameter than the cell from which it is derived. In embodiments the extracellular vesicle has a diameter from 20 nm to 1000 nm. In embodiments the targeted lipid particle comprises an apoptotic body, a fragment of a cell, a vesicle derived from a cell by direct or indirect manipulation, a vesiculated organelle, and a vesicle produced by a living cell (e.g., by direct plasma membrane budding or fusion of the late endosome with the plasma membrane). In embodiments the extracellular vesicle is derived from a living or dead organism, explanted tissues or organs, or cultured cells.

In embodiments, the targeted lipid particle comprises a nanovesicle, e.g., a cell-derived small (e.g., between 20-250 nm in diameter, or 30-150 nm in diameter) vesicle comprising a membrane that encloses an internal space, and which is generated from said cell by direct or indirect manipulation. The production of nanovesicles can, in some instances, result in the destruction of the source cell. The nanovesicle may comprise a lipid or fatty acid and polypeptide.

In embodiments, the targeted lipid particle comprises an exosome. In embodiments, the exosome is a cell-derived small (e.g., between 20-300 nm in diameter, or 40-200 nm in diameter) vesicle comprising a membrane that encloses an internal space, and which is generated from said cell by direct plasma membrane budding or by fusion of the late endosome with the plasma membrane. In embodiments, production of exosomes does not result in the destruction of the source cell. In embodiments, the exosome comprises lipid or fatty acid and polypeptide.

In some embodiments, the targeted lipid particle is derived from a source cell with a genetic modification which results in increased expression of an immunomodulatory agent. In some embodiments, the immunosuppressive agent is on an exterior surface of the cell. In some embodiments, the immunosuppressive agent is incorporated into the exterior surface of the targeted lipid particle. In some embodiments, the targeted lipid particle comprises an immunomodulatory agent attached to the surface of the solid particle by a covalent or non-covalent bond.

### Generation of Cell-Derived Particles

In some embodiments, targeted lipid particles are generated by inducing budding of an exosome, microveside, membrane vesicle, extracellular membrane vesicle, plasma membrane vesicle, giant plasma membrane vesicle, apoptotic body, mitoparticle, pyrenocyte, lysosome, or other membrane enclosed vesicle.

In some embodiments, targeted lipid particles are generated by inducing cell enucleation. Enucleation is performed using assays such as genetic, chemical (e.g., using Actinomycin D, see Bayona-Bafaluyet al., “A chemical enucleation method for the transfer of mitochondrial DNA to ρ° cells” Nucleic Acids Res. 2003 Aug. 15; 31(16): e98), or mechanical methods (e.g., squeezing or aspiration, see Lee et al., “A comparative study on the efficiency of two enucleation methods in pig somatic cell nuclear transfer effects of the squeezing and the aspiration methods.” Anim Biotechnol. 2008; 19(2):71-9), or combinations thereof.

In some embodiments, the targeted lipid particles are generated by inducing cell fragmentation. In some embodiments, cell fragmentation is performed using the following methods, including, but not limited to: chemical methods, mechanical methods (e.g., centrifugation (e.g., ultracentrifugation, or density centrifugation), freeze-thaw, or sonication), or combinations thereof.

In some embodiments, the targeted lipid particle is a microvesicle. In some embodiments the microvesicle has a diameter of about 100 nm to about 2000 nm. In some embodiments, a targeted lipid particle comprises a cell ghost. In some embodiments, a vesicle is a plasma membrane vesicle, e.g., a giant plasma membrane vesicle.

In some embodiments, a characteristic of a targeted lipid particle is described by comparison to a reference cell. In embodiments, the reference cell is the source cell. In embodiments, the reference cell is a HeLa, HEK293, HFF-1, MRC-5, WI-38, IMR 90, IMR 91, PER.C6, HT-1080, or BJ cell. In some embodiments, for example when the source cell used to make the targeted lipid particle is not available for testing after the targeted lipid particle is made, a characteristic of a population of targeted lipid particle is described by comparison to a population of reference cells, e.g., a population of source cells, or a population of HeLa, HEK293, HFF-1, MRC-5, WI-38, IMR 90, IMR 91, PER.C6, HT-1080, or BJ cells.

### CD19 Fusion Protein

Also provided herein are fusion proteins targeting CD19. In some embodiments, the CD19 binders disclosed herein are fused to an envelope glycoprotein G, H, and/or an F protein of the Paramyxoviridae family. In some embodiments the fusogen contains a Nipah virus protein F, a measles virus F protein. a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein. In some embodiments, the lipid particle contains a henipavirus envelope attachment glycoprotein G (G protein) or a biologically active portion thereof and/or a henipavirus envelope fusion glycoprotein F (F protein) or a biologically active portion thereof.

In some embodiments, the fusogen is glycoprotein GP64 of baculovirus, or glycoprotein GP64 variant E45K/T259A.

In some embodiments, the fusogen is a hemagglutinin-neurarinidase (HN) and/or fusion (F) protein (F/HN) from a respiratory paramyxovirus. In some embodiments, the respiratory paramyxovirus is a Sendai virus. The HN and F glycoproteins of Sendai viruses function to attach to sialic acids via the HN protein, and to mediate cell fusion for entry into cells via the F protein. In some embodiments, the fusogen is a F and/or HN protein from the murine parainfluenza virus type 1 (see e.g., U.S. Pat. No. 10,704,061).

In some embodiments, the lipid particle (e.g., vector) is pseudotyped with viral glycoproteins as described herein such as a NiV—F and/or NiV-G protein.

In some embodiments, the vector further comprises a vector-surface targeting moiety which specifically binds to a target ligand. In some embodiments, the vector-surface targeting moiety is a polypeptide. In some embodiments, a nucleic acid encoding the Paramyxovirus envelope protein (e.g., G protein) is modified with a targeting moiety to specifically bind to a target molecule on a target cells. In some embodiments, the targeting moiety is any targeting protein, including but not necessarily limited to antibodies and antigen binding fragments thereof.

It has been reported that the henipavirus F proteins from various species exhibit compatibility with G proteins from other species to trigger fusion (Brandel-Tretheway et al. Journal of Virology. 2019. 93(13):e00577-19). In some aspects of the provided lipid particles (e.g., lentiviral vectors), the F protein is heterologous to the G protein, i.e., the F and G proteins or biologically active portions thereof are from different henipavirus species. For example, in some embodiments the G protein is from Hendra virus and the F protein is a NiV-F as described. In other aspects, the F and/or G protein are chimeric F and/or G protein containing regions of F and/or G proteins from different species of Henipavirus. In some embodiments, replacing a portion of the F protein with amino acids from a heterologous sequence of Henipavirus results in fusion to the G protein with the heterologous sequence.

(Brandel-Tretheway et al. 2019). In some embodiments, the chimeric F and/or G protein contains an extracellular domain from one henipavirus species and a transmembrane and/or cytoplasmic domain from a different henipavirus species. For example, in some embodiments the F protein contains an extracellular domain of Hendra virus and a transmembrane/cytoplasmic domain of Nipah virus.

In some embodiments, the fusion protein contains a henipavirus envelope attachment glycoprotein G (G protein) or a biologically active portion thereof and a single domain antibody (sdAb) variable domain or a single chain variable fragment (scFv) that binds CD19 as disclosed herein. In some embodiments, the sdAb variable domain or scFv is linked directly or indirectly to the G protein. In some embodiments, the sdAb variable domain or scFv is linked to the C-terminus (C-terminal amino acid) of the G protein or the biologically active portion thereof. In some embodiments, the linkage is via a peptide linker, such as a flexible peptide linker. Table 26 provides a list of non-limiting examples of G proteins.

In some embodiments the G protein is a Henipavirus G protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein, or a biologically active portion thereof. Non-limiting examples of G proteins include those corresponding to SEQ ID NOs: 129, 138, 139, 140, and 141.

In some embodiments, the attachment G proteins are type 11 transmembrane glycoproteins containing an N-terminal cytoplasmic tail (e.g., corresponding to amino acids 1-49 of SEQ ID NO: 120), a transmembrane domain (e.g., corresponding to amino acids 50-70 of SEQ ID NO: 120), and an extracellular domain containing an extracellular stalk (e.g., corresponding to amino acids 71-187 of SEQ ID NO: 120), and a globular head (corresponding to amino acids 188-602 of SEQ ID NO: 120). In such embodiments, the N-terminal cytoplasmic domain is within the inner lumen of the lipid bilayer and the C-terminal portion is the extracellular domain that is exposed on the outside of the lipid bilayer. Regions of the stalk in the C-terminal region (e.g., corresponding to amino acids 159-167 of NiV-G) have been shown to be involved in interactions with F protein and triggering of F protein fusion (Liu et al. 2015 J of Virology 89:1838). In wild-type G protein, the globular head mediates receptor binding to henipavirus entry receptors ephrin B2 and ephrin B3, but is dispensable for membrane fusion (Brandel-Tretheway et al. Journal of Virology. 2019. 93(13)e00577-19). In some embodiments herein, tropism of the G protein is altered by linkage of the G protein or biologically active fragment thereof (e.g., cytoplasmic truncation) to a sdAb variable domain. Binding of the G protein to a binding partner can trigger fusion mediated by a compatible F protein or a biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved among henipavirus species. For example, the G proteins of NiV and HeV viruses share 79% amino acid identity. Studies have shown a high degree of compatibility among G proteins with F proteins of different species as demonstrated by heterotypic fusion activation (Brandel-Tretheway et al. Journal of Virology. 2019). As described further below, in some embodiments, a targeted lipid particle contains heterologous G and F proteins from different species.

In some embodiments, the G protein has a sequence set forth in any of SEQ ID NOs: 120, 129, 138, 139, 140, 141, 148, 156, or 158-160, or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOs: 120, 129, 138, 139, 140, 141, 148, 156, or 158-160. In some embodiments, the G protein or functionally active variant or biologically active portion is a protein that retains fusogenic activity in conjunction with a Henipavirus F protein, such as an F protein (e.g., NiV—F or HeV-F). Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell. e.g., a cell that contains a surface receptor or molecule that is recognized or bound by the antibody or antigen binding fragment thereof on the targeted lipid particle. In some embodiments, the F protein and G protein are from the same Henipavirus species (e.g., NIV-G and NiV-F). In some embodiments, the F protein and G protein are from different Henipavirus species (e.g., NiV-G and HeV-F).

In some embodiments, the G protein has the sequence of amino acids set forth in SEQ ID NOs: 120, 129, 138, 139, 140, 141, 148, 156, or 158-160, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to any one of SEQ ID NOs: 120, 129, 138, 139, 140, 141, 148, 156. or 158-160 and retains fusogenic activity in conjunction with a Henipavirus F protein (e.g., NiV—F or HeV-F). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to any one of SEQ ID NOs: 120, 129, 138, 139, 140, 141, 148, 156, or 158-160and retains fusogenic activity in conjunction with a Henipavirus F protein (e.g., NiV—F or HeV-F).

Reference to retaining fusogenic activity includes activity (in conjunction with a Henipavirus F protein) that is at or about 10% to at or about 150% or more of the level or degree of binding of the corresponding wild-type G protein, such as set forth in any one of SEQ ID NOs: 120, 129, 138, 139, 140, 141,148, 156, or 158-160, such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 80% of the level or degree of fusogenic activity of the corresponding wild-type G protein, such as at least or at least about 85% of the level or degree of fusogenic activity of the corresponding wild-type G protein, such as at least or at least about 90% of the level or degree of fusogenic activity of the corresponding wild-type G protein, such as at least or at least about 95% of the level or degree of fusogenic activity of the corresponding wild-type G protein, such as at least or at least about 100% of the level or degree of fusogenic activity of the corresponding wild-type G protein, or such as at least or at least about 120% of the level or degree of fusogenic activity of the corresponding wild-type G protein.

In some embodiments, the G protein is a mutant G protein that is a functionally active variant or biologically active portion containing one or more amino acid mutations, such as one or more amino acid insertions, deletions, substitutions, or truncations. In some embodiments, the mutations described herein relate to amino acid insertions, deletions, substitutions, or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein, or biologically active portions thereof. In some embodiments, the wild-type G protein has the sequence set forth in any one of SEQ ID NOs: 120, 129, 138, 139, 140, 141, 148, 156, or 158-160.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NV-G), a wild-type Cedar (CedPV) virus G-protein, a wild-type Mojiang virus G-protein, or a wild-type bat Paramyxovirus G-protein. In some embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NOs: 120, 129, 138, 139, 140, 141, 148, 156, or 158-10. In some embodiments, the mutant G protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acid(s) at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:120. SEQ ID NO:138. or SEQ ID NO:148, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:120. SEQ ID NO:138, or SEQ ID NO:148.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein (SEQ ID NO:120, SEQ ID NO:138, or SEQ ID NO:148), up to 6 contiguous amino acid residues at or near the N-terminus of the wild-type NiV-G protein (SEQ ID NO:120, SEQ ID NO:138, or SEQ ID NO:148), up to 7 contiguous amino acid residues at or near the N-terminus of the wild-type NiV-G protein (SEQ ID NO:120, SEQ ID NO:138, or SEQ ID NO:148), up to 8 contiguous amino acid residues at or near the N-terminus of the wild-type NiV-G protein (SEQ ID NO:120, SEQ ID NO:138, or SEQ ID NO:148), up to 9 contiguous amino acid residues at or near the N-terminus of the wild-type NiV-G protein (SEQ ID NO:120, SEQ ID NO:138, or SEQ ID NO:148), up to 10 contiguous amino acid residues at or near the N-terminus of the wild-type NiV-G protein (SEQ ID NO:120, SEQ ID NO:138, or SEQ ID NO:148), up to 11 contiguous amino acid residues at or near the N-terminus of the wild-type NiV-G protein (SEQ ID NO:120, SEQ ID NO:138, or SEQ ID NO:148), up to 12 contiguous amino acid residues at or near the N-terminus of the wild-type NiV-G protein (SEQ ID NO:120, SEQ ID NO:138, or SEQ ID NO:148), up to 13 contiguous amino acid residues at or near the N-terminus of the wild-type NiV-G protein (SEQ ID NO:120, SEQ ID NO:138, or SEQ ID NO:148), up to 14 contiguous amino acid residues at or near the N-terminus of the wild-type NiV-G protein (SEQ ID NO:120, SEQ ID NO:138, or SEQ ID NO:148), up to 15 contiguous amino acid residues at or near the N-terminus of the wild-type NiV-G protein (SEQ ID NO:120, SEQ ID NO:138, or SEQ ID NO:148), up to 16 contiguous amino acid residues at or near the N-terminus of the wild-type NiV-G protein (SEQ ID NO:120, SEQ ID NO:138, or SEQ ID NO:148), up to 17 contiguous amino acid residues at or near the N-terminus of the wild-type NiV-G protein (SEQ ID NO:120, SEQ ID NO:138, or SEQ ID NO:148), up to 18 contiguous amino acid residues at or near the N-terminus of the wild-type NaV-G protein (SEQ ID NO:120. SEQ ID NO:138, or SEQ ID NO:148), up to 19 contiguous amino acid residues at or near the N-terminus of the wild-type NiV-G protein (SEQ ID NO:120, SEQ ID NO:138, or SEQ ID NO:148), up to 20 contiguous amino acid residues at or near the N-terminus of the wild-type NiV-G protein (SEQ ID NO:120, SEQ ID NO:138, or SEQ ID NO:148), up to 21 contiguous amino acid residues at or near the N-terminus of the wild-type NiV-G protein (SEQ ID NO:120, SEQ ID NO:138, or SEQ ID NO:148), up to 22 contiguous amino acid residues at or near the N-terminus of the wild-type NiV-G protein (SEQ ID NO:120, SEQ ID NO:138, or SEQ ID NO:148), up to 23 contiguous amino acid residues at or near the N-terminus of the wild-type NiV-G protein (SEQ ID NO:120, SEQ ID NO:138, or SEQ ID NO:148), up to 24 contiguous amino acid residues at or near the N-terminus of the wild-type NiV-G protein (SEQ ID NO:120, SEQ ID NO:138, or SEQ ID NO:148), up to 25 contiguous amino acid residues at or near the N-terminus of the wild-type NiV-G protein (SEQ ID NO:120, SEQ ID NO:138, or SEQ ID NO:148), up to 26 contiguous amino acid residues at or near the N-terminus of the wild-type NiV-G protein (SEQ ID NO:120, SEQ ID NO:138, or SEQ ID NO:148). up to 27 contiguous amino acid residues at or near the N-terminus of the wild-type NiV-G protein (SEQ ID NO:120, SEQ ID NO:138, or SEQ ID NO:148), up to 28 contiguous amino acid residues at or near the N-terminus of the wild-type NiV-G protein (SEQ ID NO:120, SEQ ID NO:138, or SEQ ID NO:148). up to 29 contiguous amino acid residues at or near the N-terminus of the wild-type NiV-G protein (SEQ ID NO:120, SEQ ID NO:138, or SEQ ID NO:148), up to 30 contiguous amino acid residues at or near the N-terminus of the wild-type NiV-G protein (SEQ ID NO:120, SEQ ID NO:138, or SEQ ID NO:148), up to 31 contiguous amino acid residues at or near the N-terminus of the wild-type NiV-G protein (SEQ ID NO:120, SEQ ID NO:138, or SEQ ID NO:148), up to 32 contiguous amino acid residues at or near the N-terminus of the wild-type NiV-G protein (SEQ ID NO:120, SEQ ID NO:138, or SEQ ID NO:148), up to 33 contiguous amino acid residues at or near the N-terminus of the wild-type NiV-G protein (SEQ ID NO:120, SEQ ID NO:138, or SEQ ID NO:148), up to 34 contiguous amino acid residues at or near the N-terminus of the wild-type N,V-G protein (SEQ ID NO:120, SEQ ID NO:138, or SEQ ID NO:148), up to 35 contiguous amino acid residues at or near the N-terminus of the wild-type NiV-G protein (SEQ ID NO:120, SEQ ID NO:138, or SEQ ID NO:148), up to 36 contiguous amino acid residues at or near the N-terminus of the wild-type NiV-G protein (SEQ ID NO:120, SEQ ID NO:138, or SEQ ID NO:148), up to 37 contiguous amino acid residues at or near the N-terminus of the wild-type NiV-G protein (SEQ ID NO:120, SEQ ID NO:138, or SEQ ID NO:148), up to 38 contiguous amino acid residues at or near the N-terminus of the wild-type NiV-G protein (SEQ ID NO:120, SEQ ID NO:138, or SEQ ID NO:148), up to 39 contiguous amino acid residues at or near the N-terminus of the wild-type NiV-G protein (SEQ ID NO:120. SEQ ID NO:138, or SEQ ID NO:148), up to 40 contiguous amino acid residues at or near the N-terminus of the wild-type NiV-G protein (SEQ ID NO:120. SEQ ID NO:138, or SEQ ID NO:148), up to 41 contiguous amino acid residues at or near the N-terminus of the wild-type NiV-G protein (SEQ ID NO:120. SEQ ID NO:138, or SEQ ID NO:148), up to 42 contiguous amino acid residues at or near the N-terminus of the wild-type NiV-G protein (SEQ ID NO:120, SEQ ID NO:138, or SEQ ID NO:148), up to 43 contiguous amino acid residues at or near the N-terminus of the wild-type NiV-G protein (SEQ ID NO:120, SEQ ID NO:138, or SEQ ID NO:148), up to 44 contiguous amino acid residues at or near the N-terminus of the wild-type NiV-G protein (SEQ ID NO:120, SEQ ID NO: 138, or SEQ ID NO: 148), or up to 45 contiguous amino acid residues at or near the N-terminus of the wild-type NiV-G protein (SEQ ID NO:120, SEQ ID NO:138, or SEQ ID NO:148).

In some embodiments, the NiV-G protein is a biologically active portion that does not contain a cytoplasmic domain. In some embodiments, the NIV-G protein without the cytoplasmic domain is encoded by SEQ ID NO:142.

In some embodiments, the mutant NiV-G protein comprises a sequence set forth in any of SEQ ID NOs: 121-126, 149-154, 132, 142, or 157, or is a functional variant thereof that has an amino acid sequence having at least at or about 80%, at least at or about 81%, at least at or about 82%, at least at or about 83%, at least at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NOs: 121-126, 149-154, 132, 142, or 157.

In some embodiments, the mutant NiV-G protein has a 5 amino acid truncation at or near the N-terminus of the wild-type NiV-G protein (SEQ ID NO:120, SEQ ID NO:138, or SEQ ID NO:148), such as set forth in SEQ ID NO:121 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:121, or as set forth in SEQ ID NO:149 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:149 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:149.

In some embodiments, the mutant NiV-G protein has a 10 amino acid truncation at or near the N-terminus of the wild-type NiV-G protein (SEQ ID NO:120, SEQ ID NO:138, or SEQ ID NO:148), such as set forth in SEQ ID NO:122 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:122, or such as set forth in SEQ ID NO:150 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:150.

In some embodiments, the mutant NiV-G protein has a 15 amino acid truncation at or near the N-terminus of the wild-type NiV-G protein (SEQ ID NO:120, SEQ ID NO:138, or SEQ ID NO:148), such as set forth in SEQ ID NO:123 or a functional variant thereof that has an amino acid sequence having at least at or about 80%, at least at or about 81%, at least at or about 82%, at least at or about 83%, at least at or about 84%, at least at or about 85%, at least at or about 88%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:123, or such as set forth in SEQ ID NO:151 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:151.

In some embodiments, the mutant NiV-G protein has a 20 amino acid truncation at or near the N-terminus of the wild-type NiV-G protein (SEQ ID NO:120, SEQ ID NO:138, or SEQ ID NO:148) such as set forth in SEQ ID NO:124, or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least or about 96%, at least at or about 97%, at least at or about 98%, or at least at or about 99% sequence identity to SEQ ID NO:124, or such as set forth in SEQ ID NO:152 or afunctional variant thereof having at least at or about 80%, at least at or about 81%, at least at or about 82%, at least at or about 83%, at least or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:152.

In some embodiments, the mutant NiV-G protein has a 25 amino acid truncation at or near the N-terminus of the wild-type NiV-G protein (SEQ ID NO:120, SEQ ID NO:138, or SEQ ID NO:148), such as set forth in SEQ ID NO:125 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least or about 96%, at least at or about 97%, at least at or about 98%, or at least at or about 99% sequence identity to SEQ ID NO:125, or such as set forth in SEQ ID NO:153 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:153.

In some embodiments, the mutant NiV-G protein has a 30 amino acid truncation at or near the N-terminus of the wild-type NiV-G protein (SEQ ID NO:120, SEQ ID NO:138, or SEQ ID NO:148), such as set forth in SEQ ID NO:126 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:126, or such as set forth in SEQ ID NO:154 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:154.

In some embodiments, the mutant NIV-G protein has a 33 amino acid truncation at or near the N-terminus of the wild-type NiV-G protein (SEQ ID NO:120, SEQ ID NO:138, or SEQ ID NO:148) or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:132, or such as set forth in SEQ ID NO:155 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:155.

In some embodiments, the mutant NiV-G protein has a 34 amino acid truncation at or near the N-terminus of the wild-type NiV-G protein (SEQ ID NO:120, SEQ ID NO:138, or SEQ ID NO:148), such as set forth in SEQ ID NO:132 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:132, or such as set forth in SEQ ID NO:155 or a functional variant thereof having at least at or about 80%, at least at or about 81%, at least at or about 82%: at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:155.

In some embodiments, the NiV-G protein has a 34 amino acid truncation at or near the N-terminus of the wild-type NiV-G protein (SEQ ID NO: 120, SEQ ID NO: 138, or SEQ ID NO: 148) and one or more amino acid substitutions corresponding to amino acid substitutions selected from E501A, W504A, Q530A, and E533A with reference to the numbering set forth in SEQ ID NO: 138.

In some embodiments, the mutant NiV-G protein lacks the N-terminal cytoplasmic domain of the wild-type NiV-G protein (SEQ ID NO:120, SEQ ID NO:138, or SEQ ID NO:148), such as set forth in SEQ ID NO:142 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:142.

In some embodiments, the mutant G protein is a mutant HeV-G protein that has the sequence set forth in SEQ ID NO:129 or 156, or is a functional variant or biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least or about 96%, at least at or about 97%, at least at or about 98%, or at least at or about 99% sequence identity to SEQ ID NO:129 or 156.

In some embodiments, the G protein is a mutant HeV-G protein that is a biologically active portion of a wild-type HeV-G. In some embodiments, the biologically active portion is an N-terminally truncated fragment. In some embodiments, the mutant HeV-G protein is truncated and lacks up to 5 contiguous amino acid residues at or near the N-terminus of the wild-type HeV-G protein (SEQ ID NO; 129 or 156), up to 6 contiguous amino acid residues at or near the N-terminus of the wild-type HeV-G protein (SEQ ID NO:129 or 156), up to 7 contiguous amino acid residues at or near the N-terminus of the wild-type HeV-G protein (SEQ ID NO:129 or 156), up to 8 contiguous amino acid residues at or near the N-terminus of the wild-type HeV-G protein (SEQ ID NO:129 or 156), up to 9 contiguous amino acid residues at or near the N-terminus of the wild-type HeV-G protein (SEQ ID NO:129 or 156), up to 10 contiguous amino acid residues at or near the N-terminus of the wild-type HeV-G protein (SEQ ID NO:129 or 156), up to 11 contiguous amino acid residues at or near the N-terminus of the wild-type HeV-G protein (SEQ ID NO:129 or 156), up to 12 contiguous amino acid residues at or near the N-terminus of the wild-type HeV-G protein (SEQ ID NO:129 or 156), up to 13 contiguous amino acid residues at or near the N-terminus of the wild-type HeV-G protein (SEQ ID NO:129 or 156). up to 14 contiguous amino acid residues at or near the N-terminus of the wild-type HeV-G protein (SEQ ID NO:129 or 156), up to 15 contiguous amino acid residues at or near the N-terminus of the wild-type HeV-G protein (SEQ ID NO:129 or 156), up to 16 contiguous amino acid residues at or near the N-terminus of the wild-type HeV-G protein (SEQ ID NO:129 or 156), up to 17 contiguous amino acid residues at or near the N-terminus of the wild-type HeV-G protein (SEQ ID NO:129 or 156), up to 18 contiguous amino acid residues at or near the N-terminus of the wild-type HeV-G protein (SEQ ID NO:129 or 156), up to 19 contiguous amino acid residues at or near the N-terminus of the wild-type HeV-G protein (SEQ ID NO:129 or 156), up to 20 contiguous amino acid residues at or near the N-terminus of the wild-type HeV-G protein (SEQ ID NO:129 or 156), up to 21 contiguous amino acid residues at or near the N-terminus of the wild-type HeV-G protein (SEQ ID NO:129 or 156), up to 22 contiguous amino acid residues at or near the N-terminus of the wild-type HeV-G protein (SEQ ID NO:129 or 156), up to 23 contiguous amino acid residues at or near the N-terminus of the wild-type HeV-G protein (SEQ ID NO:129 or 156), up to 24 contiguous amino acid residues at or near the N-terminus of the wild-type HeV-G protein (SEQ ID NO:129 or 156), up to 25 contiguous amino acid residues at or near the N-terminus of the wild-type HeV-G protein (SEQ ID NO:129 or 156), up to 26 contiguous amino acid residues at or near the N-terminus of the wild-type HeV-G protein (SEQ ID NO:129 or 156), up to 27 contiguous amino acid residues at or near the N-terminus of the wild-type HeV-G protein (SEQ ID NO:129 or 156), up to 28 contiguous amino acid residues at or near the N-terminus of the wild-type HeV-G protein (SEQ ID NO:129 or 156), up to 29 contiguous amino acid residues at or near the N-terminus of the wild-type HeV-G protein (SEQ ID NO:129 or 156), up to 30 contiguous amino acid residues at or near the N-terminus of the wild-type HeV-G protein (SEQ ID NO:129 or 156), up to 31 contiguous amino acid residues at or near the N-terminus of the wild-type HeV-G protein (SEQ ID NO:129 or 156), up to 32 contiguous amino acid residues at or near the N-terminus of the wild-type HeV-G protein (SEQ ID NO:129 or 156), up to 33 contiguous amino acid residues at or near the N-terminus of the wild-type HeV-G protein (SEQ ID NO:129 or 156), up to 34 contiguous amino acid residues at or near the N-terminus of the wild-type HeV-G protein (SEQ ID NO:129 or 156), up to 35 contiguous amino acid residues at or near the N-terminus of the wild-type HeV-G protein (SEQ ID NO:129 or 156), up to 36 contiguous amino acid residues at or near the N-terminus of the wild-type HeV-G protein (SEQ ID NO:129 or 156), up to 37 contiguous amino acid residues at or near the N-terminus of the wild-type HeV-G protein (SEQ ID NO:129 or 156), up to 38 contiguous amino acid residues at or near the N-terminus of the wild-type HeV-G protein (SEQ ID NO:129 or 156), up to 39 contiguous amino acid residues at or near the N-terminus of the wild-type HeV-G protein (SEQ ID NO:129 or 156), up to 40 contiguous amino acid residues at or near the N-terminus of the wild-type HeV-G protein (SEQ ID NO:129 or 156), up to 41 contiguous amino acid residues at or near the N-terminus of the wild-type HeV-G protein (SEQ ID NO:129 or 156), up to 42 contiguous amino acid residues at or near the N-terminus of the wild-type HeV-G protein (SEQ ID NO:129 or 156). up to 43 contiguous amino acid residues at or near the N-terminus of the wild-type HeV-G protein (SEQ ID NO:129 or 156), up to 44 contiguous amino acid residues at or near the N-terminus of the wild-type HeV-G protein (SEQ ID NO:129 or 156), or up to 45 contiguous amino acid residues at or near the N-terminus of the wild-type HeV-G protein (SEQ ID NO:129 or 156).

In some embodiments, the HeV-G protein is a biologically active portion that does not contain a cytoplasmic domain. In some embodiments, the mutant HeV-G protein lacks the N-terminal cytoplasmic domain of the wild-type HeV-G protein (SEQ ID NO:129 or 156), such as set forth in SEQ ID NO:143 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:143.

In some embodiments, the G protein or the functionally active variant or biologically active portion thereof binds to Ephrin B2 or Ephrin B3. In some aspects, the G protein has the sequence of amino acids set forth in any one of SEQ ID NO:120, SEQ ID NO:129, SEQ ID NO:138, SEQ ID NO:139, SEQ ID NO:148, SEQ ID NO:140, or SEQ ID NO:141, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, and retains binding to Ephrin B2 or B3.

Reference to retaining binding to Ephrin B2 or B3 includes binding that is at least or at least about 5% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, 10% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, 15% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, 20% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, 25% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion. 30% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof. 35% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, 40% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, 45% of the level or degree of binding of the corresponding wild-type G protein, such as set forth in SEQ ID NO: 120, SEQ ID NO:129, SEQ ID NO:138, SEQ ID NO:139. SEQ ID NO:148, SEQ ID NO:140, or SEQ ID NO:141, or a functionally active variant or biologically active portion thereof, 50% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, 55% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof. 60% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, 65% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, 70% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 75% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 80% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 85% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 90% of the level or degree of binding of the corresponding wild-type G protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof, or such as at least or at least about 95% of the level or degree of binding of the corresponding wild-type protein, such as set forth in SEQ ID NO: 120, SEQ ID NO:129, SEQ ID NO:138, SEQ ID NO:139, SEQ ID NO:148, SEQ ID NO:140, or SEQ ID NO:141, or a functionally active variant or biologically active portion thereof.

In some embodiments, the G protein is NiV-G or a functionally active variant or biologically active portion thereof and binds to Ephrin B2 or Ephrin B3. In some aspects, the NiV-G has the sequence of amino acids set forth in SEQ ID NO:120, SEQ ID NO:138, or SEQ ID NO:148, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:120, SEQ ID NO:138, or SEQ ID NO:148 and retains binding to Ephrin B2 or B3. Exemplary biologically active portions include N-terminally truncated variants lacking all or a portion of the cytoplasmic domain, e.g., 1 or more, such as 1 to 49 contiguous N-terminal amino acid residues, e.g., set forth in any one of SEQ ID NOs: 121-126, 142, and 149-154.

Reference to retaining binding to Ephrin B2 or B3 includes binding that is at least or at least about 5% of the level or degree of binding of the corresponding wild-type NiV-G, such as set forth in SEQ ID NO:120, SEQ ID NO:138, or SEQ ID NO:148, 10% of the level or degree of binding of the corresponding wild-type NiV-G, such as set forth in SEQ ID NO:120, SEQ ID NO:138, or SEQ ID NO:148, 15% of the level or degree of binding of the corresponding wild-type NiV-G, such as set forth in SEQ ID NO:120, SEQ ID NO:138. or SEQ ID NO:148, 20% of the level or degree of binding of the corresponding wild-type NiV-G, such as set forth in SEQ ID NO:120, SEQ ID NO:138, or SEQ ID NO:148, 25% of the level or degree of binding of the corresponding wild-type NiV-G, such as set forth in SEQ ID NO:120, SEQ ID NO:138, or SEQ ID NO:148, 30% of the level or degree of binding of the corresponding wild-type NiV-G, such as set forth in S SEQ ID NO:120, SEQ ID NO:138, or SEQ ID NO:148, 35% of the level or degree of binding of the corresponding wild-type NiV-G, such as set forth in SEQ ID NO:120, SEQ ID NO:138, or SEQ ID NO:148, 40% of the level or degree of binding of the corresponding wild-type NiV-G, such as set forth in SEQ ID NO:120, SEQ ID NO:138, or SEQ ID NO:148, 45% of the level or degree of binding of the corresponding wild-type NiV-G, such as set forth in SEQ ID NO:120, SEQ ID NO:138, or SEQ ID NO:148, 50% of the level or degree of binding of the corresponding wild-type NiV-G, such as set forth in SEQ ID NO:120, SEQ ID NO:138. or SEQ ID NO:148, 55% of the level or degree of binding of the corresponding wild-type NiV-G, such as set forth in SEQ ID NO:120, SEQ ID NO:138, or SEQ ID NO:148, 60% of the level or degree of binding of the corresponding wild-type NiV-G, such as set forth in SEQ ID NO:120, SEQ ID NO:138, or SEQ ID NO:148, 65% of the level or degree of binding of the corresponding wild-type NiV-G, such as set forth In SEQ ID NO:120, SEQ ID NO:138, or SEQ ID NO:148, 70% of the level or degree of binding of the corresponding wild-type NIV-G, such as set forth in SEQ ID NO:120, SEQ ID NO:138, or SEQ ID NO:148, such as at least or at least about 75% of the level or degree of binding of the corresponding wild-type NiV-G, such as set forth in SEQ ID NO:120, SEQ ID NO:138, or SEQ ID NO:148, such as at least or at least about 80% of the level or degree of binding of the corresponding wild-type NIV-G, such as set forth in SEQ ID NO:120, SEQ ID NO:138, or SEQ ID NO:148, such as at least or at least about 85% of the level or degree of binding of the corresponding wild-type NiV-G, such as set forth in SEQ ID NO:120, SEQ ID NO:138, or SEQ ID NO:148, such as at least or at least about 90% of the level or degree of binding of the corresponding wild-type NiV-G, such as set forth In SEQ ID NO:120, SEQ ID NO:138, or SEQ ID NO:148, or such as at least or at least about 95% of the level or degree of binding of the corresponding wild-type NiV-G, such as set forth in SEQ ID NO:120, SEQ ID NO:138, or SEQ ID NO:148.

In some embodiments, the G protein is HeV-G or a functionally active variant or biologically active portion thereof and binds to Ephrin B2 or Ephrin B3. In some aspects, the HeV-G has the sequence of amino acids set forth in SEQ ID NO:129 or 156, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:129 or 156 and retains binding to Ephrin B2 or B3. Exemplary biologically active portions include N-terminally truncated variants lacking all or a portion of the cytoplasmic domain, e.g., 1 or more, such as 1 to 49 contiguous N-terminal amino acid residues, e.g., set forth in any one of SEQ ID NO:143.

Reference to retaining binding to Ephin B2 or B3 includes binding that is at least or at least about 5% of the level or degree of binding of the corresponding wild-type HeV-G, such as set forth in SEQ ID NO:129 or 156, 10% of the level or degree of binding of the corresponding wild-type HeV-G, such as set forth in SEQ ID NO:129 or 156, 15% of the level or degree of binding of the corresponding wild-type HeV-G, such as set forth in SEQ ID NO:129 or 156, 20% of the level or degree of binding of the corresponding wild-type HeV-G, such as set forth in SEQ ID NO:129 or 156, 25% of the level or degree of binding of the corresponding wild-type HeV-G, such as set forth in SEQ ID NO:129 or 156, 30% of the level or degree of binding of the corresponding wild-type HeV-G, such as set forth in SEQ ID NO:129 or 156, 35% of the level or degree of binding of the corresponding wild-type HeV-G, such as set forth in SEQ ID NO:129 or 156, 40% of the level or degree of binding of the corresponding wild-type HeV-G, such as set forth in SEQ ID NO:129 or 156.45% of the level or degree of binding of the corresponding wild-type HeV-G, such as set forth in SEQ ID NO:129 or 156, 50% of the level or degree of binding of the corresponding wild-type HeV-G, such as set forth in SEQ ID NO:129 or 156, 55% of the level or degree of binding of the corresponding wild-type HeV-G, such as set forth in SEQ ID NO:129 or 156, 60% of the level or degree of binding of the corresponding wild-type HeV-G, such as set forth in SEQ ID NO:129 or 156, 65% of the level or degree of binding of the corresponding wild-type HeV-G, such as set forth in SEQ ID NO:129 or 156, 70% of the level or degree of binding of the corresponding wild-type HeV-G, such as set forth in SEQ ID NO:129 or 156, such as at least or at least about 75% of the level or degree of binding of the corresponding wild-type HeV-G, such as set forth in SEQ ID NO:129 or 156, such as at least or at least about 80% of the level or degree of binding of the corresponding wild-type NIV-G, such as set forth in SEQ ID NO:129 or 156, such as at least or at least about 85% of the level or degree of binding of the corresponding wild-type HeV-G, such as set forth in SEQ ID NO:129 or 156, such as at least or at least about 90% of the level or degree of binding of the corresponding wild-type HeV-G, such as set forth in SEQ ID NO:129 or 156, or such as at least or at least about 95% of the level or degree of binding of the corresponding wild-type HeV-G, such as set forth in SEQ ID NO:129 or 156.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such as to reduce the binding to at least one of Ephrin B2 or Ephrin B3. In some embodiments, the mutations described herein interfere with natural receptor recognition.

In some embodiments, the mutant NiV-G protein or the biologically active portion thereof is truncated and lacks up to 5 contiguous amino acid residues at or near the N-terminus of the wild-type NiV-G protein (SEQ ID NO:138). 6 contiguous amino acid residues at or near the N-terminus of the wild-type NiV-G protein (SEQ ID NO:138), 7 contiguous amino acid residues at or near the N-terminus of the wild-type NiV-G protein (SEQ ID NO:138), 8 contiguous amino acid residues at or near the N-terminus of the wild-type NiV-G protein (SEQ ID NO:138), 9 contiguous amino acid residues at or near the N-terminus of the wild-type NiV-G protein (SEQ ID NO:138), contiguous amino acid residues at or near the N-terminus of the wild-type NiV-G protein (SEQ ID NO:138), 11 contiguous amino acid residues at or near the N-terminus of the wild-type NIV-G protein (SEQ ID NO:138), 12 contiguous amino acid residues at or near the N-terminus of the wild-type NiV-G protein (SEQ ID NO:138), 13 contiguous amino acid residues at or near the N-terminus of the wild-type NiV-G protein (SEQ ID NO:138), 14 contiguous amino acid residues at or near the N-terminus of the wild-type NiV-G protein (SEQ ID NO:138), 15 contiguous amino acid residues at or near the N-terminus of the wild-type NiV-G protein (SEQ ID NO:138), 16 contiguous amino acid residues at or near the N-terminus of the wild-type NiV-G protein (SEQ ID NO:138), 17 contiguous amino acid residues at or near the N-terminus of the wild-type NiV-G protein (SEQ ID NO:138), 18 contiguous amino acid residues at or near the N-terminus of the wild-type NiV-G protein (SEQ ID NO:138), 19 contiguous amino acid residues at or near the N-terminus of the wild-type NiV-G protein (SEQ ID NO:138), 20 contiguous amino acid residues at or near the N-terminus of the wild-type NiV-G protein (SEQ ID NO:138), 21 contiguous amino acid residues at or near the N-terminus of the wild-type NiV-G protein (SEQ ID NO:138). 22 contiguous amino acid residues at or near the N-terminus of the wild-type NiV-G protein (SEQ ID NO:138), 23 contiguous amino acid residues at or near the N-terminus of the wild-type NiV-G protein (SEQ ID NO:138), 24 contiguous amino acid residues at or near the N-terminus of the wild-type NiV-G protein (SEQ ID NO:138), contiguous amino acid residues at or near the N-terminus of the wild-type NiV-G protein (SEQ ID NO:138), 26 contiguous amino acid residues at or near the N-terminus of the wild-type NiV-G protein (SEQ ID NO:138), 27 contiguous amino acid residues at or near the N-terminus of the wild-type NiV-G protein (SEQ ID NO:138), 28 contiguous amino acid residues at or near the N-terminus of the wild-type NiV-G protein (SEQ ID NO:138), 29 contiguous amino acid residues at or near the N-terminus of the wild-type NiV-G protein (SEQ ID NO:138), 30 contiguous amino acid residues at or near the N-terminus of the wild-type NiV-G protein (SEQ ID NO:138), 31 contiguous amino acid residues at or near the N-terminus of the wild-type NiV-G protein (SEQ ID NO:138). 32 contiguous amino acid residues at or near the N-terminus of the wild-type NiV-G protein (SEQ ID NO:138), 33 contiguous amino acid residues at or near the N-terminus of the wild-type NiV-G protein (SEQ ID NO:138), 34 contiguous amino acid residues at or near the N-terminus of the wild-type NiV-G protein (SEQ ID NO:138), 35 contiguous amino acid residues at or near the N-terminus of the wild-type NiV-G protein (SEQ ID NO:138), 36 contiguous amino acid residues at or near the N-terminus of the wild-type NiV-G protein (SEQ ID NO:138), 37 contiguous amino acid residues at or near the N-terminus of the wild-type NiV-G protein (SEQ ID NO:138), 38 contiguous amino acid residues at or near the N-terminus of the wild-type NiV-G protein (SEQ ID NO:138), 39 contiguous amino acid residues at or near the N-terminus of the wild-type NiV-G protein (SEQ ID NO:138), or 40 contiguous amino acid residues at or near the N-terminus of the wild-type NiV-G protein (SEQ ID NO:138).

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, QS30A, and E533A with reference to numbering set forth in SEQ ID NO:138.

In some embodiments, the G protein is a mutant G protein containing one or more amino acid substitutions selected from the group consisting of E501A, W504A, Q530A, and E533A with reference to numbering set forth in SEQ ID NO:138. In some embodiments, the G protein is a mutant G protein that contains one or more amino acid substitutions selected from the group consisting of E501A, W504A, Q530A, and E533A with reference to SEQ ID NO:138 or a biologically active portion thereof containing an N-terminal truncation. In some embodiments, the G protein is a mutant G protein that contains one or more amino acid substitutions selected from the group consisting of E501A, W504A, Q530A, and E533A in combination with any one of the N-terminal truncations disclosed above with reference to SEQ ID NO:138 or a biologically active portion thereof. In some embodiments, any of the mutant G proteins described above contains one, two, three, or all four amino acid selected from the group consisting of E501A, W504A, Q530A, and E533A with reference to numbering set forth in SEQ ID NO:138, in all pairwise and triple combinations thereof.

In some embodiments, the mutant NiV-G protein has the amino acid sequence set forth in SEQ ID NO: 127 or 155 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence to identity to SEQ ID NO: 127 or 155. In some embodiments, the G protein has the sequence of amino acids set forth in SEQ ID NO: 127 or 155.

In some embodiments, the targeted envelope protein contains a G protein or a functionally active variant or biologically active portion thereof and an antibody or antigen binding fragment thereof, in which the targeted envelope protein exhibits increased binding for another molecule that is different from the native binding partner of a wild-type G protein. In some embodiments, the antibody or antigen binding fragment thereof is a scFv or sdAb. In some embodiments, the other molecule is a protein expressed on the surface of desired target cell. In some embodiments the other molecule that is different from the native binding partner of a wild-type G protein is CD19. In some embodiments, the increased binding to the other molecule is increased by greater than at or about 25%, at or about 30%, at or about 40%, at or about 50%, at or about 60%, at or about 70%, at or about 80%, at or about 90%, or at or about 100%. In some embodiments, the binding confers re-targeted binding compared to the binding of a wild-type G protein in which a new or different binding activity is conferred.

In some embodiments, the C-terminus of the antibody or antigen binding fragment thereof is attached to the C-terminus of the G protein or biologically active portion thereof. In some embodiments, the N-terminus end of the antibody or antigen binding fragment thereof is exposed on the exterior surface of the lipid bilayer. In some embodiments, the N-terminus end of the antibody or antigen binding fragment thereof binds to a cell surface molecule of a target cell. In some embodiments, the antibody or antigen binding fragment thereof specifically binds to a cell surface molecule present on a target cell. In some embodiments, the cell surface molecule is a protein, glycan, lipid, or low molecular weight molecule. In some embodiments, the cell surface molecule is CD19.

In some embodiments, the cell surface molecule of a target cel is an antigen or portion thereof. In some embodiments, the antibody or antigen binding fragment thereof is an antibody having a single monomeric domain antigen binding/recognition domain that is able to bind selectively to a specific antigen. In some embodiments, the single domain antibody binds an antigen present on a target cell.

Exemplary cells include immune effector cells, peripheral blood mononuclear cells (PBMC) such as lymphocytes (T cells, B cells, natural killer cells) and monocytes, granulocytes (neutrophils, basophils, eosinophils), macrophages, dendritic cells, cytotoxic T lymphocytes, polymorphonuclear cells (also known as PMN, PML, or PMNL), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes.

In some embodiments, the target cell is a cell of a target tissue. In some embodiments, the target tissue is liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g., hepatocyte), or a cardiac cell (e.g., cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g., a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+B cell, a CD19+B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacytoid dendritic cell, a CD11c+ cell, a CD11b+ cell, a splenocyte, a B cell, a hepatocyte, an endothelial cell, or a non-cancerous cell. In some embodiments, the cell surface molecule is any one of CD8.

In some embodiments, the G protein or functionally active variant or biologically active portion thereof is linked directly to the sdAb variable domain (e.g., a VHH) or scFv. In some embodiments, the targeted envelope protein is a fusion protein that has the following structure: (N′-single domain antibody-C′)-(C′-G protein-N′). In some embodiments, the targeted envelope protein is a fusion protein that has the following structure: (N′-scFv-C′)-(C′-G protein-N′).

In some embodiments, the G protein or functionally active variant or biologically active portion thereof is linked indirectly via a linker to the sdAb variable domain or scFv. In some embodiments, the linker is a peptide linker. In some embodiments, the linker is a chemical linker.

In some embodiments, the linker is a peptide linker and the targeted envelope protein is a fusion protein containing the G protein or functionally active variant or biologically active portion thereof linked via a peptide linker to the sdAb variable domain or svFv. In some embodiments, the targeted envelope protein is a fusion protein that has the following structure: (N′-single domain antibody-C′)-Linker-(C′-G protein-N′). In some embodiments, the targeted envelope protein is a fusion protein that has the following structure: (N′-scFv-C′)-Linker-(C′-G protein-N′). In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids. 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids. 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids. 20 to 24 amino acids, 24 to 65 amino acids, 24 to 60 amino acids, 24 to 56 amino acids, 24 to 52 amino acids. 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids. 28 to 52 amino acids, 28 to 48 amino acids, 28 to 44 amino acids. 28 to 40 amino acids, 28 to 36 amino acids, 28 to 34 amino acids, 28 to 32 amino acids, 32 to 65 amino acids. 32 to 60 amino acids. 32 to 56 amino acids, 32 to 52 amino acids, 32 to 48 amino acids, 32 to 44 amino acids, 32 to 40 amino acids, 32 to 38 amino adds, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino adds, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino adds, 58 to 65 amino acids. 58 to 60 amino acids, or 60 to 65 amino acids. In some embodiments, the peptide linker is a polypeptide that is 3, 4, 5, 6, 7, 8, 9, 10, 11, 12, 13. 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In some embodiments, the linker is a flexible peptide linker. In some such embodiments, the linker is 1-20 amino adds, such as 1-20 amino adds comprising glycine. In some embodiments, the linker is 1-20 amino acids, such as 1-20 amino acids comprising glycine and serine. In some embodiments, the linker is a flexible peptide linker containing amino acids Glycine and Serine, referred to as GS-linkers.

In some embodiments, the peptide linker includes the sequences GS, GGS, GGGGS (SEQ ID NO:147), GGGGGS (SEQ ID NO:145) or combinations thereof. In some embodiments, the polypeptide linker has the sequence (GGS)n, (SEQ ID NO:231) wherein n is 1 to 10. In some embodiments, the polypeptide linker has the sequence (GGGGS)n, (SEQ ID NO:146) wherein n is 1 to 10. In some embodiments, the polypeptide linker has the sequence (GGGGGS)n (SEQ ID NO:137), wherein n is 1 to 6.

Also provided herein are polynucleotides comprising a nucleic acid sequence encoding a targeted envelope protein. In some embodiments, the polynucleotides comprise a nucleic acid sequence encoding a G protein or biologically active portion thereof. In some embodiments, the polynucleotides further comprise a nucleic acid sequence encoding a single domain antibody (sdAb) variable domain or scFv or biologically active portion thereof. The polynucleotides may include a sequence of nucleotides encoding any of the targeted envelope proteins described above. In some embodiments, the polynucleotide is a synthetic nucleic acid. Also provided are expression vectors containing any of the provided polynucleotides.

In some embodiments, expression of natural or synthetic nucleic acids is achieved by operably linking a nucleic acid encoding the gene of interest to a promoter and incorporating the construct into an expression vector. In some embodiments, vectors are suitable for replication and integration in eukaryotes. In some embodiments, cloning vectors contain transcription and translation terminators, initiation sequences, and promoters useful for expression of the desired nucleic acid sequence. In some of any embodiments, a plasmid comprises a promoter suitable for expression in a cell.

In some embodiments, the polynucleotides contain at least one promoter that is operatively linked to control expression of the targeted envelope protein containing the G protein and the single domain antibody (sdAb) variable domain or scFv. For expression of the targeted envelope protein, at least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

In some embodiments, additional promoter elements, e.g., enhancers, regulate the frequency of transcriptional initiation. In some embodiments, additional promoter elements are located in the region 30-110 bp upstream of the start site, although a number of promoters have been shown to contain functional elements downstream of the start site as well. In some embodiments, spacing between promoter elements frequently is flexible, so that promoter function is preserved when elements are inverted or moved relative to one another. In some embodiments, such as with the thymidine kinase (tk) promoter, the spacing between promoter elements is increased to 50 bp apart before activity begins to decline. In some embodiments, depending on the promoter, individual elements can function either cooperatively or independently to activate transcription.

In some embodiments, a promoter is one naturally associated with a gene or polynucleotide sequence, as is obtained by isolating the 5′ non-coding sequences located upstream of the coding segment and/or exon. In some embodiments, such a promoter is referred to as “endogenous.” In some embodiments, an enhancer is one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein.

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor-Ia (EF-1 a). In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In some embodiments, the promoter is an inducible promoter. In some embodiments, the inducible promoter provides a molecular switch capable of turning on expression of the polynucleotide sequence to which it is operatively linked when such expression is desired, or turning off the expression when expression is not desired. In some embodiments, inducible promoters comprise a metallothionine promoter, a glucocorticoid promoter, a progesterone promoter, and a tetracycline promoter.

In some embodiments, exogenously controlled inducible promoters are used to regulate expression of the G protein and single domain antibody (sdAb) variable domain or scFv. For example, radiation-inducible promoters, heat-inducible promoters, and/or drug-inducible promoters are used to selectively drive transgene expression in, for example, targeted regions. In such embodiments, the location, duration, and level of transgene expression are regulated by the administration of the exogenous source of induction.

In some embodiments, expression of the targeted envelope protein containing a G protein and single domain antibody (sdAb) variable domain or scFv is regulated using a drug-inducible promoter. For example, in some embodiments, the promoter, enhancer, or transactivator comprises a Lac operator sequence, a tetracycline operator sequence, a galactose operator sequence, a doxycycline operator sequence, a rapamycin operator sequence, a tamoxifen operator sequence, or a hormone-responsive operator sequence, or an analog thereof. In some instances. the inducible promoter comprises a tetracycline response element (TRE). In some embodiments, the inducible promoter comprises an estrogen response element (ERE), which can activate gene expression in the presence of tamoxifen. In some instances, a drug-inducible element, such as a TRE, is combined with a selected promoter to enhance transcription in the presence of drug, such as doxycycline. In some embodiments, the drug-inducible promoter Is a small molecule-inducible promoter.

In some embodiments, any of the provided polynucleotides are modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In order to assess the expression of the targeted envelope protein, the expression vector to be introduced into a cell can also contain either a selectable marker gene or a reporter gene or both to facilitate identification and selection of expressing particles, e.g., viral particles. In other embodiments, the selectable marker is carried on a separate piece of DNA and used in a co-transfection procedure. In some embodiments, both selectable markers and reporter genes are flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000, FEBS Lett. 479:79-82). Suitable expression systems are well known and may be prepared using well known techniques or obtained commercially. Internal deletion constructs are generated using unique internal restriction sites or by partial digestion of non-unique restriction sites. Constructs may then be transfected into cells that display high levels of the desired polynucleotide and/or polypeptide expression. In general, the construct with the minimal 5′ flanking region showing the highest level of expression of reporter gene is identified as the promoter. In some embodiments, such promoter regions are linked to a reporter gene and used to evaluate agents for the ability to modulate promoter-driven transcription.

### Delivery of CAR by Targeted Vector

Provided herein are methods of administering a targeted lipid particle (e.g., vector) is targeting a cell. Exemplary cells include immune effector cells, peripheral blood mononuclear cells (PBMC) such as lymphocytes (T cells, B cells, natural killer cells) and monocytes, granulocytes (neutrophils, basophils, eosinophils), macrophages, dendritic cells, cytotoxic T lymphocytes, polymorphonuclear cells (also known as PMN, PML, or PMNL). stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cels, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells. peripheral blood stem cells (PBSCs), neurons, neural progenitor cells, pancreatic beta cells, glial cells, or hepatocytes. In some embodiments, the target cell is contacted with a targeted lipid particle.

In some embodiments, the cell is an immune cell. In some embodiments, the immune cell is a NK cell, a T cell, a macrophage, or a monocyte. In some embodiments, the immune cell is a T cel. In some embodiments, the T cell is a CD3+ T cell, a CD4+ T cell, a CDS+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T cell, an effector memory T cell, an effector memory T cell expressing CD45RA (TEMRA cell), a tissue-resident memory (Trm) cell, a virtual memory T cell, an innate memory T cell, a memory stem cell (Tse), or a γδ T cell. In some embodiments, the T cell is a cytotoxic T cell, a helper T cell, a memory T cell, a regulatory T cell, or a tumor infiltrating lymphocyte.

In some embodiments, the T cell is a human T cell. In some embodiments, the T cell is an autologous T cell. In other embodiments, the T cell is an allogeneic T cell. In some embodiments, the allogeneic T cell is a primary T cell. In some embodiments, the allogeneic T cell has been differentiated from an embryonic stem cell (ESC) or an induced pluripotent stem cell (iPSC).

In some embodiments, the method of administering a targeted lipid particle (e.g., vector) targeting a T cell comprise contacting a T cell with a targeted lipid particle comprising a targeting antibody or antigen binding fragment thereof and an exogenous agent to a subject as disclosed herein. In some embodiments, the exogenous agent is a polynucleotide encoding a CAR (e.g., CAR transgene). In some embodiments the method comprises a) obtaining whole blood from the subject; b) collecting the fraction of blood containing leukocyte components including T cells; c) contacting the leukocyte components including T cells with a composition comprising the lentiviral vector to create a transfection mixture; and d) reinfusing the contacted leukocyte components including T cells and/or the transfection mixture to the subject, thereby administering the lipid particle and the exogenous agent to the subject. In some embodiments, the T cells (e.g., CD4+ or CD8+ T cells) are not activated during the method. In some embodiments, step (c) of the method is carried out for no more than 24 hours, e.g., no more than 20, 16, 12, 8. 6, 5, 4, 3, 2, or 1 hour.

In some embodiments, the method according to the present disclosure is capable of delivering a targeted lipid particle to an ex vivo system. The method includes the use of a combination of various apheresis machine hardware components, a software control module, and a sensor module to measure citrate or other solute levels in-line to ensure the maximum accuracy and safety of treatment prescriptions, and the use of replacement fluids designed to fully exploit the design of the system according to the present methods. In some embodiments, components described for one system according to the present invention are implemented within other systems according to the present invention as well.

In some embodiments, the method for administration of the targeted lipid particle (e.g., a lentiviral vector) to the subject comprises the use of a blood processing set for obtaining whole blood from the subject, a separation chamber for collecting the fraction of blood containing leukocyte components including T cells, a contacting container for contacting the T cells with the composition comprising the lentiviral vector, and a further fluid circuit for reinfusion of T cells to the patient. In some embodiments, the method further comprises any of i) a washing component for concentrating T cells, and ii) a sensor and/or module for monitoring cell density and/or concentration. In some embodiments, the methods allow processing of blood directly from the patient, transduction with the lentiviral vector, and reinfusion directly to the patient without any steps of selection for T cells. Further, in some embodiments the methods are carried out without cryopreserving or freezing any cells before or between any one or more of the steps, such that there is no step of formulating cells with a cryoprotectant, e.g., DMSO. In some embodiments, the provided methods do not include a lymphodepletion regimen. In some embodiments, the method including steps (a)-(d) are carried out for a time of no more than 24 hours, such as between 2 hours and 12 hours, for example 3 hours to 6 hours.

In some embodiments, the method is performed in-line (or in situ). In some embodiments, the method is performed in a closed fluid circuit, or a functionally closed fluid circuit. In some embodiments, each of steps (a)-(d) are performed in-line in a closed fluid circuit in which all parts of the system are operably connected, such as via at least one tubing line. In some embodiments, the system is sterile. In some embodiments, the closed fluid circuit is sterile.

Also provided herein are systems for administration of a targeted lipid particle (e.g., lentiviral vector) comprising a C targeting antibody and an exogenous agent as herein disclosed to a subject.

In some embodiments, the targeted lipid particles (e.g., targeted viral vectors) provided herein, or pharmaceutical compositions thereof as described herein are administered to a subject, e.g., a mammal, e.g., a human. In such embodiments, the subject is at risk of, has a symptom of, or is diagnosed with or identified as having, a particular disease or condition. In one embodiment, the subject has cancer. In one embodiment, the subject has an infectious disease. In some embodiments, the targeted viral vector contains nucleic acid sequences encoding an exogenous agent for treating the disease or condition in the subject. In some embodiments, the exogenous agent comprises a polynucleotide encoding a CAR. For example, the exogenous agent is a polynucleotide encoding a CAR that targets or is specific for a protein of a neoplastic cells and the targeted lipid particle is administered to a subject for treating a tumor or cancer in the subject. In some examples, the exogenous agent is an inflammatory mediator or immune molecule, such as a cytokine, and targeted lipid particle is administered to a subject for treating any condition in which it is desired to modulate (e.g., increase) the immune response, such as a cancer or infectious disease. In some embodiments, the targeted viral vector is administered in an effective amount or dose to effect treatment of the disease, condition or disorder. Provided herein are uses of any of the provided targeted viral vector in such methods and treatments, and in the preparation of a medicament in order to cany out such therapeutic methods. In some embodiments, the methods are carried out by administering the targeted viral vector or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent.

In some embodiments, the provided methods or uses involve administration of a pharmaceutical composition comprising oral, inhaled, transdermal or parenteral (including intravenous, intratumoral, intraperitoneal, intramuscular, intracavity, and subcutaneous) administration. In some embodiments, the targeted viral vector is administered alone or formulated as a pharmaceutical composition. In some embodiments, the targeted viral vector or compositions described herein are administered to a subject, e.g., a mammal, e.g., a human. In some of any embodiments, the subject is at risk of, has a symptom of, or is diagnosed with or identified as having, a particular disease or condition (e.g., a disease or condition described herein). In some embodiments, the disease is a disease or disorder. In some embodiments, the disease is a B cel malignancy.

In some embodiments, the targeted lipid particles is administered in the form of a unit-dose composition, such as a unit dose oral, parenteral, transdermal, or inhaled composition. In some embodiments, the compositions are prepared by admixture and are adapted for oral, inhaled, transdermal, or parenteral administration, and as such are in the form of tablets, capsules, oral liquid preparations, powders, granules, lozenges, reconstitutable powders, injectable, and infusable solutions or suspensions, or suppositories or aerosols.

In some embodiments, the regimen of administration may affect what constitutes an effective amount. In some embodiments, the therapeutic formulations are administered to the subject either prior to or after a diagnosis of disease. In some embodiments, several divided dosages, as well as staggered dosages are administered daily or sequentially, or the dose is continuously infused, or is a bolus injection. In some embodiments, the dosages of the therapeutic formulations are proportionally increased or decreased as indicated by the exigencies of the therapeutic or prophylactic situation.

In some embodiments, the administration of the compositions of the present disclosure to a subject, preferably a mammal, more preferably a human, is carried out using known procedures, at dosages and for periods of time effective to prevent or treat disease. In some embodiments, an effective amount of the targeted lipid particle of the disclosure necessary to achieve a therapeutic effect may vary according to factors such as the activity of the particular lipid particle employed; the time of administration; the rate of excretion; the duration of the treatment; other drugs, compounds or materials used in combination with the targeted lipid particle of the disclosure; the state of the disease or disorder, age, sex, weight, condition, general health and prior medical history of the subject being treated, and like factors well-known in the medical arts. In some embodiments, the dosage regimens are adjusted to provide the optimum therapeutic response. In some embodiments, several divided doses are administered daily, or the dose is proportionally reduced as indicated by the exigencies of the therapeutic situation. One of ordinary skill in the art would be able to study the relevant factors and make the determination regarding the effective amount of the therapeutic targeted lipid particle of the disclosure without undue experimentation.

In some embodiments, dosage levels of the targeted lipid particles in the pharmaceutical compositions of this disclosure are varied so as to obtain an amount that is effective to achieve the desired therapeutic response for a particular subject. composition, and mode of administration, without being toxic to the subject.

A medical doctor, e.g., physician or veterinarian, having ordinary skill in the art may readily determine and prescribe the effective amount of the pharmaceutical composition required. In some embodiments, the physician or veterinarian could start doses of the targeted lipid particles of the disclosure employed in the pharmaceutical composition at levels lower than that required in order to achieve the desired therapeutic effect and gradually increase the dosage until the desired effect is achieved.

In some embodiments, the term “container” includes any receptacle for holding the pharmaceutical composition. In some embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. It should be understood that the instructions for use of the pharmaceutical composition is contained on the packaging containing the pharmaceutical composition, and as such the instructions form an increased functional relationship to the packaged product. In some embodiments, instructions may contain information pertaining to the pharmaceutical composition's ability to perform its intended function, e.g., treating or preventing a disease in a subject, or delivering an imaging or diagnostic agent to a subject.

In some embodiments, routes of administration of any of the compositions disclosed herein include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, itrathecal, subcutaneous, intramuscular, intradermal, intra-arterial, intravenous, intrabronchial, inhalation, and topical administration.

In some of any embodiments, suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration, and the like.

In some embodiments, the targeted lipid particle composition comprising an exogenous agent or cargo, is used to deliver such exogenous agent or cargo to a cell tissue or subject. In some embodiments, delivery of a cargo by administration of a targeted lipid particle composition described herein may modify cellular protein expression levels. In some embodiments, the administered composition directs upregulation (via expression in the cell, delivery in the cell, or induction within the cell) of one or more cargo (e.g., a polypeptide or mRNA) that provide a functional activity which is substantially absent or reduced in the cell in which the polypeptide is delivered. In some embodiments, the missing functional activity is enzymatic, structural, or regulatory in nature, In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some embodiments, the upregulated functional activity is enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the targeted lipid particle composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the targeted viral vector composition comprises an exogenous protein), the effect lasts for less than 1, 2, 3, 4, 5, 6, or 7 days, 2, 3, or 4 weeks, or 1, 2, 3, 6, or 12 months.

In some of any embodiments, the targeted lipid particle composition described herein is delivered ex-vivo to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered to an ex vivo tissue that is in an injured state (e.g., from trauma, disease, hypoxia, ischemia or other damage).

In some embodiments, the composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the composition is delivered, administered, or contacted with a cell, e.g., a cell preparation. In some embodiments, the cell preparation is a cell therapy preparation (a cell preparation intended for administration to a human subject). In embodiments, the cell preparation comprises cells expressing a chimeric antigen receptor (CAR), e.g., expressing a recombinant CAR. The cells expressing the CAR is, e.g., T cells. Natural Killer (NK) cells, cytotoxic T lymphocytes (CTL), regulatory T cells. In embodiments, the cell preparation is a neural stem cell preparation. In embodiments, the cell preparation is a mesenchymal stem cell (MSC) preparation. In embodiments, the cell preparation is a hematopoietic stem cell (HSC) preparation. In embodiments, the cell preparation is an islet cell preparation.

In some embodiments, the viral vector comprising an anti-CD8 or anti-CD4 sdAb or scFv and an exogenous agent described herein is used to deliver a CAR. In some embodiments, the viral vector transduces a cell expressing CD4 or CD8 (e.g., a CD4+ T cell or a CD8+ T cell) and the transduced cell expresses and amplifies the CAR. The resulting CAR T cells then mediate targeted cell killing. Thus, the disclosure includes the use of viral vector comprising an anti-CD8 or anti-CD4 scFv or sdAb fusogen construct to elicit an immune response specific to the antigen t0 binding moiety of the CAR. In some embodiments, the CAR is used to target a CD19 tumor antigen as herein disclosed. In some embodiments, the CAR is used to target a CD19 tumor antigen and another cell surface molecule selected from CD5, CD19, CD20, CD22, CD23, CD30, CD33, CD38, CD70, CD123, CD138, GPRC5D, LeY, NKG2D, WT1, GD2, HER2, EGFR, EGFRvill, B7H3, PSMA, PSCA, CAIX, CD171, CEA, CSPG4, EPHA2, FAP, FRa, IL-13Rα, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor. Go, GD3. HMW-MAA, IL-11Ra, KDR, Lewis Y, L1-cell adhesion molecule, MADE-A1, Oncofetal antigen (h5T4), TAG-72, CD19122, Syndecan 1, or BCMA. In some embodiments, the CAR is engineered to comprise an intracellular signaling domain of the T cell antigen receptor complex zeta chain (e.g., CD3 zeta). In some embodiments, the intracellular domain is selected from a CD137 (4-1BB) signaling domain, a CD28 signaling domain, and a CD3zeta signaling domain.

Methods for introducing a CAR construct or producing a CAR-T cells are well known to those skilled in the art. Detailed descriptions are disclosed herein and are found, for example, in Vormittag et al., Curr Opin Biotechnol, 2018, 53, 162-181; and Eyquem et al., Nature, 2017, 543, 113-117.

### Cells Expressing CAR

In some aspects, the present technology provides cells expressing one or more chimeric antigen receptor (CAR) on the surface of the cell. These cells are referred to as “engineered cells.” In some embodiments, one or more CARs are delivered to a cell as herein disclosed, e.g., through a viral vector, and the cell expresses the CARs on its surface.

In some embodiments, the cell is an immune cell. In some embodiments, the immune cell is a NK cell, a T cell, a Macrophage, or a Monocyte. In some embodiments, the cell is a T cell. In some embodiments, the T cell is The method of claim 59 or 60, wherein the T cell is a CD3+ T cell, a CD4+ T cell, a CDS+ T cell, a naive T cell, a regulatory T (Treg) cell, a non-regulatory T cell, a Th cell, a Th2 cell, a Th9 cell, a Th17 cell, a T-follicular helper (Tfh) cell, a cytotoxic T lymphocyte (CTL), an effector T (Teff) cell, a central memory T cell, an effector memory T cell, an effector memory T cell expressing CD45RA (TEMRA cell), a tissue-resident memory (Trm) cell, a virtual memory T cell, an innate memory T cell, a memory stem cell (Tse), or a γδ T cell. In some embodiments, the T cell is a cytotoxic T cell. In some embodiments, the T cell is a helper T cell. In some embodiments, the T cell is a memory T cell. In some embodiments, the T cell is a regulatory T cell. In some embodiments, the T cell is a tumor infiltrating lymphocyte. In some embodiments, the T cell is a human T cell. In some embodiments, the T cell is an autologous T cell. In other embodiments, the T cell is an allogeneic T cell. In some embodiments, the allogeneic T cell is a primary T cell. In some embodiments, the allogeneic T cell has been differentiated from an embryonic stem cell (ESC) or an induced pluripotent stem cell (iPSC).

In some embodiments, two or more cells expressing CARs of the present disclosure are in a composition. In some embodiments, the composition comprises cells expressing the same CAR targeting CD19. In other embodiments, the composition comprises cells expressing bispecific CARs targeting CD19 and one of CD5, CD19, CD20, CD22, CD23, CD30, CD33, CD38, CD70, CD123, CD138, GPRC5D, LeY, NKG2D, WT1, GD2, HER2, EGFR, EGFRvII, B7H3, PSMA, PSCA, CAIX, CD171, CEA, CSPG4, EPHA2, FAP, FRa, IL-13Rα, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-82, erb-B, FBP, Fetal acetylcholine e receptor, G_{D2}, G_{D3}, HMW-MAA, IL-11Rα, KDR, Lewis Y, L1-cell adhesion molecule. MADE-A1, Oncofetal antigen (h5T4), TAG-72, CD19/22, Syndecan 1, or BCMA. In some embodiments, the composition comprises cells expressing a CAR targeting CD19 and cells expressing a bispecific CAR targeting CD19 and one of CD5, CD19, CD20, CD22, CD23, CD30, CD33, CD38, CD70, CD123, CD138, GPRC5D, LeY, NKG2D, WT1, G_{D}2, HER2, EGFR, EGFRvIII, B7H3, PSMA, PSCA, CAIX, CD171, CEA, CSPG4, EPHA2, FAP, FRa, IL-13Rα, Mesothelin, MUC1, MUC16, ROR1, C-Met, CD133, Ep-CAM, GPC3, HPV16, IL13Ra2, MAGEA3. MAGEA4, MART1, NY-ESO, VEGFR2, α-Falate, CD24, CD44v7/8, EGP-2, EGP-40, erb-B2, erb-B, FBP, Fetal acetylcholine e receptor, G_{D2}, G_{D3}, HMW-MAA, IL-11Ra, KDR, Lewis Y, L1-cell adhesion molecule, MADE-A1, Oncofetal antigen (h5T4), TAG-72, CD19122, Syndecan 1, or BCMA. In other embodiments, the cells of the composition express the same CARs, e.g., CARs targeting the same cell surface molecule. In other embodiments, the cells of the composition express different CARs, e.g., CARs targeting different cell surface molecules.

In some embodiments, the cells used in connection with the provided uses, articles of manufacture and compositions include cells employing single-targeting strategies, such as expression of one genetically engineered receptor herein disclosed, e.g., a CAR, on the cell. In some embodiments, the cells used in connection with the provided methods, uses, articles of manufacture and compositions include cells employing multi-targeting strategies, such as expression of two or more genetically engineered receptors herein disclosed, e.g., CARs, on the cell, each recognizing the same of a different antigen and typically each including a different intracellular signaling component. Such multi-targeting strategies are described, for example, in WO 2014055668 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells include a receptor expressing a first genetically engineered antigen receptor (e.g., CAR) which is capable of inducing an activating or stimulatory signal to the cell, generally upon specific binding to the antigen or cell surface molecule recognized by the first receptor, e.g., the first antigen. In some embodiments, the cell further includes a second genetically engineered antigen receptor (e.g., CAR), e.g., a chimeric costimulatory receptor, which is capable of inducing a costimulatory signal to the immune cell, generally upon specific binding to a second antigen or cell surface molecule recognized by the second receptor. In some embodiments, the first antigen and second antigen are the same. In some embodiments, the first antigen and second antigen are different. In some embodiments, the first antigen Is CD)19 and the second antigen is one of CD5, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAX, CD171, CEA, CSPG4, EPHA2, FAP, FRa, IL-13Rα, Mesothelin, MUC1, MUC16, ROR1, C-Met. CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, G_{D}2, Ga3. HMW-MAA, IL-11Ra. KDR, Lewis Y, L1-cell adhesion molecule, MADE-A1, Oncofetal antigen (h5T4), TAG-72, CD19/22, Syndecan 1, or BCMA

In some embodiments, the first and/or second genetically engineered antigen receptor (e.g., CAR) is capable of inducing an activating signal to the cell. In some embodiments, the receptor includes an intracellular signaling component containing ITAM or ITAM-like motifs. In some embodiments, the activation induced by the first receptor involves a signal transduction or change in protein expression in the cell resulting in initiation of an immune response, such as ITAM phosphorylation and/or initiation of IT AM-mediated signal transduction cascade, formation of an immunological synapse and/or clustering of molecules near the bound receptor (e.g., CD4 or CD8, etc.), activation of one or more transcription factors, such as NF—KB and/or AP-1, and/or induction of gene expression of factors such as cytokines, proliferation, and/or survival.

In some embodiments, the first and/or second receptor includes intracellular signaling domains or regions of costimulatory receptors such as CD28, CD137 (4-1BB), OX40, and/or ICOS. In some embodiments, the first and second receptor include an intracellular signaling domain of a costimulatory receptor that are different. In one embodiment, the first receptor contains a CD28 costimulatory signaling region and the second receptor contain a 4-IBB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or IT AM-like motifs and the second receptor contains an intracellular signaling domain of a costimulatory receptor. The costimulatory signal in combination with the activating signal induced in the same cell is one that results in an immune response, such as a robust and sustained immune response, such as increased gene expression, secretion of cytokines and other factors, and T cell mediated effector functions such as cell killing.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a cell that expresses the first and second antigens.

In some embodiments, the two receptors induce, respectively, an activating and an inhibitory signal to the cell, such that binding by one of the receptors to its antigen activates the cell or induces a response. but binding by the second inhibitory receptor to its antigen induces a signal that suppresses or dampens that response. Examples are combinations of activating CARs and inhibitory CARs or iCARs. Such a strategy is used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such embodiments, by requiring ligation of two separate and individually specific antigen receptors, specificity, selectivity, and/or efficacy is improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

### Hypoimmune CAR-T Cell

In some embodiments, the present disclosure is directed to pluripotent stem cells (e.g., pluripotent stem cells and induced pluripotent stem cells (iPSCs)), differentiated cells derived from such pluripotent stem cells (such as, but not limited to, T cells and NK cells), and primary cells (such as, but not limited to, primary T cells and primary NK cells) that express a CAR. In some embodiments, the pluripotent stem cells, differentiated cells derived therefrom, such as T cells and NK cells, and primary cells such as primary T cells and primary NK cells, are engineered for reduced expression or lack of expression of MHC class I and/or MHC class II human leukocyte antigens, and in some instances, for reduced expression or lack of expression of a T-cell receptor (TCR) complex. In some embodiments, the hypoimmune (HIP) T cells and primary T cells overexpress CD47 and a chimeric antigen receptor (CAR) in addition to reduced expression or lack of expression of MHC class I and/or MHC class II human leukocyte antigens, and have reduced expression or lack expression of a T-cell receptor (TCR) complex. In some embodiments, the CAR comprises an antigen binding domain that binds to CD19. In some embodiments, the CAR comprises an antigen binding domain that specifically binds to CD19 and a second antigen binding domain that specifically binds to CD5, CD19, CD20, CD22, CD23, C030, CD33, CD38, CD70, CD123. CD138, GPRC5D, LeY, NKG2D, WT1, G_{D}2, HER2, EGFR. EGFRvIII, B7H3, PSMA, PSCA, CAIX, CD171, CEA, CSPG4, EPHA2, FAP, FRoe, IL-13Rα, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, G_{D}2, Goa. HMW-MAA, IL-11Ro, KDR, Lewis Y, L1-cell adhesion molecule, MADE-A1, Oncofetal antigen (h5T4), TAG-72, CD19122. Syndecan 1, or BCMA. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, engineered and/or hypoimmune (HIP) T cells and primary T cells overexpress CD47 and one or more chimeric antigen receptor (CAR), and include a genomic modification of the B2M gene. In some embodiments, engineered and/or hypoimmune (HIP) T cells and primary T cells overexpress CD47 and include a genomic modification of the CIITA gene. In some embodiments, engineered and/or hypoimmune (HIP) T cells and primary T cells overexpress CD47 and one or more CAR, and include a genomic modification of the TRAC gene. In some embodiments, engineered and/or hypoimmune (HIP) T cells and primary T cells overexpress CD47 and one or more CAR, and include a genomic modification of the TRB gene. In some embodiments, engineered and/or hypoimmune (HIP) T cells and primary T cells overexpress CD47 and one or more CAR, and include one or more genomic modifications selected from the group consisting of the 82M, CIITA, TRAC, and TRB genes. In some embodiments, engineered and/or hypoimmune (HIP) T cells and primary T cells overexpress CD47 and one or more CAR, and include genomic modifications of the B2M, CIITA, TRAC, and TRB genes. In some embodiments, the cells are B2M^{-/-}, −CIIT^{-/-}, TRAC^{-/-}, CD47tg cells that also express CARs. In some embodiments, engineered and/or hypoimmune (HIP) T cells are produced by differentiating induced pluripotent stem cells such as engineered and/or hypoimmunogenic induced pluripotent stem cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the engineered and/or hypoimmune (HIP) T cells and primary T cells are B2M^{-/-}, CIITA^{-/-}, TRB^{-/-}, CD47tg cels that also express CARs. In some embodiments, the cells are B2M^{-/-}, CIITA^{-/-}, TRAC^{-/-}, TRB^{-/-}, CD47tg cells that also express CARs. In some embodiments, the cells are B2M^{indel/indel}, CIITA^{indel/indel}, TRAC^{indel/indel}, CD47tg cells that also express CARs. In some embodiments, the cells are B2M^{indel/indel}, CIITA^{indel/indel}, TRB^{indel/indel}, CD47tg cells that also express CARs. In some embodiments, the cells are B2M^{indel/indel}, CIITA^{indel/indel}, TRAC^{indel/indel}, TRB^{indel/indel}, CD47tg cells that also express CARs. In some embodiments, the engineered or modified cells described are pluripotent stem cells, induced pluripotent stem cells, NK cells differentiated from such pluripotent stem cells and induced pluripotent stem cells, T cells differentiated from such pluripotent stem cells and induced pluripotent stem cells, or primary T cells. Non-limiting examples of primary T cells include CD3+ T cells, CD4+ T cells, CDS+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tern) cells, effector memory T (Tern) cells, effector memory T cells express CD45RA (TEMRA cells), tissue-resident memory (Trm) cells, virtual memory T cels, innate memory T cells, memory stem cell (Tse), γδ T cells, and any other subtype of T cells. In some embodiments, the primary T cells are selected from a group that includes cytotoxic T-cells, helper T-cells, memory T-cells, regulatory T-cells, tumor infiltrating lymphocytes, and combinations thereof. Non-limiting examples of NK cells and primary NK cells include immature NK cells and mature NK cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the primary T cells are from a pool of primary T cells from one or more donor subjects that are different than the recipient subject (e.g., the patient administered the cells). In some embodiments, the primary T cells are obtained from 1, 2, 3, 4, 5, 6, 7, 8, 9, 10, 20, 50, 100 or more donor subjects and pooled together. In some embodiments, the primary T cells are obtained from 1 or more, 2 or more, 3 or more, 4 or more, 5 or more, 6 or more, 7 or more, 8 or more, 9 or more, 10, or more 20 or more. 50 or more, or 100 or more donor subjects and pooled together. In some embodiments, the primary T cells are harvested from one or a plurality of individuals, and in some instances, the primary T cells or the pool of primary T cells are cultured in vitro. In some embodiments, the primary T cells or the pool of primary T cells are engineered to exogenously express CD47 and cultured in vitro.

In some embodiments, the primary T cells or the pool of primary T cells are engineered to express a chimeric antigen receptor (CAR) as herein disclosed. In some embodiments, the CAR is any known to those skilled in the art.

In some embodiments, the primary T cells or the pool of primary T cells are engineered to exhibit reduced expression of an endogenous T cell receptor compared to unmodified primary T cells. In some embodiments, the primary T cells or the pool of primary T cells are engineered to exhibit reduced expression of CTLA-4, PD-1, or both CTLA-4 and PD-1, as compared to unmodified primary T cells. Methods of genetically modifying a cell including a T cell are described in detail, for example, in WO2020/018620 and WO2016/183041, the disclosures of which are herein incorporated by reference in their entireties, including the tables, appendices, sequence listing and figures.

In some embodiments, the cells derived from primary T cells comprise reduced expression of an endogenous T cell receptor, for example by disruption of an endogenous T cell receptor gene (e.g., T cell receptor alpha constant region (TRAC) or T cell receptor beta constant region (TRB)). In some embodiments, an exogenous nucleic acid encoding a polypeptide as disclosed herein (e.g., a chimeric antigen receptor, CD47, or another tolerogenic factor disclosed herein) is inserted at the disrupted T cell receptor gene. In some embodiments, an exogenous nucleic acid encoding a polypeptide is inserted at a TRAC or a TRB gene locus.

In some embodiments, the cells derived from primary T cells comprise reduced expression of cytotoxic T-lymphocyte-associated protein 4 (CTLA4) and/or programmed cell death (PD1). Methods of reducing or eliminating expression of CTLA4, PD1 and both CTLA4 and PD1 are any recognized by those skilled in the art, such as but not limited to, genetic modification technologies that utilize rare-cutting endonucleases and RNA silencing or RNA interference technologies. Non-limiting examples of a rare-cutting endonuclease include any Cas protein, T ALEN, zinc finger nuclease, meganuclease, and/or homing endonuclease. In some embodiments, an exogenous nucleic acid encoding a polypeptide as disclosed herein (e.g., a chimeric antigen receptor, CD47, or another tolerogenic factor disclosed herein) is inserted at a CTLA4 and/or PD1 gene locus. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell. In some embodiments, the exogenous polynucleotide is inserted into at least one allele of the cell using transfection or transduction, for example, with a vector as disclosed herein. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, a CD47 transgene is inserted into a pre-selected locus of the cell. In some embodiments, a CD47 transgene is inserted into a random locus of the cell. In some embodiments, a transgene encoding a CAR as disclosed herein is to inserted into a pre-selected locus of the cell. In some embodiments, a transgene encoding a CAR is inserted into a random locus of the cell. In some embodiments, a CD47 transgene and a transgene encoding a CAR are inserted into a pre-selected locus of the cell. In some embodiments, a transgene encoding a CAR is inserted into a random or pre-selected locus of the cell, including a safe harbor locus, via viral vector transduction/integration. In some embodiments, a CD47 transgene and a transgene encoding a CAR are inserted into a random or pre-selected locus of the cell, including a safe harbor locus, via viral vector transduction/integration. In some embodiments, the vector is a self-inactivating lentiviral vector pseudotyped with a vesicular stomatitis VSVG envelope. In some embodiments, the transgene encoding a CAR is inserted into at least one allele of the cell using viral transduction. In some embodiments, the exogenous polynucleotide is inserted into at least one allele of the cell using a lentivirus based viral vector. In some embodiments, the random and/or pre-selected locus is a safe harbor or target locus. Non-limiting examples of a safe harbor locus include, but are not limited to, a CCR5 gene locus, a PPP1R12C (also known as AAVS1) gene locus, and a CLYBL gene locus, a Rosa gene locus (e.g., ROSA26 gene locus). Non-limiting examples of a target locus include, but are not limited to, a CXCR4 gene locus, an albumin gene locus, a SHS231 gene locus, an F3 gene locus (also known as CD142), a MICA gene locus, a MICB gene locus, a LRP1 gene locus (also known as a CD91 gene locus), a HMGB1 gene locus, an ABO gene locus, ad RHD gene locus, a FUT1 locus, and a KDM5D gene locus. In some embodiments, the CD47 transgene is inserted in Introns 1 or 2 for PPP1R12C (i.e., AAVS1) or CCR5. In some embodiments, the CD47 transgene is inserted in Exons 1 or 2 or 3 for CCR5. In some embodiments, the CD47 transgene is inserted in intron 2 for CLYBL. In some embodiments, the CD47 transgene is inserted in a 500 bp window in Ch-4:58,976,613 (i.e., SHS231). In some embodiments, the CD47 trans gene is inserted in any suitable region of the aforementioned safe harbor or target loci that allows for expression of the exogenous polynucleotide, including, for example, an intron, an exon or a coding sequence region in a safe harbor or target locus. In some embodiments, the pre-selected locus is selected from the group consisting of the B2M locus, the CIITA locus, the TRAC locus, and the TRB locus. In some embodiments, the preselected locus is the B2Mlocus. In some embodiments, the pre-selected locus is the CIITA locus. In some embodiments, the pre-selected locus is the TRAC locus. In some embodiments, the pre-selected locus is the TRB locus. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, a CD47 transgene and a transgene encoding a CAR are inserted into the same locus. In some embodiments, a CD47 transgene and a transgene encoding a CAR are inserted into different loci. In many instances, a CD47 transgene is inserted into a safe harbor or target locus. In many instances, a transgene encoding a CAR is inserted into a safe harbor or target locus. In some instances, a CD47 transgene is inserted into a B2M locus. In some instances, a trans gene encoding a CAR is inserted into a B2M locus. In some instances, a CD47 transgene is inserted into a CIITA locus. In some instances, a transgene encoding a CAR is inserted into a CIITA locus. In some instances, a CD47 transgene is inserted into a TRAC locus. In some instances, a transgene encoding a CAR is inserted into a TRAC locus. In many other instances, a CD47 transgene is inserted into a TRB locus. In many other instances, a trans gene encoding a CAR is inserted into a TRB locus. In some embodiments, a CD47 transgene and a transgene encoding a CAR are inserted into a safe harbor or target locus (e.g., a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus.

In some embodiments, a CD47 transgene and a transgene encoding a CAR are inserted into a safe harbor or target locus. In some embodiments, a CD47 transgene and a trans gene encoding a CAR are controlled by a single promoter and are inserted into a safe harbor or target locus. In some embodiments, a CD47 to transgene and a transgene encoding a CAR are controlled by their own promoters and are inserted into a safe harbor or target locus. In some embodiments, a CD47 transgene and a transgene encoding a CAR are inserted into a TRAC locus. In some embodiments, a CD47 transgene and a transgene encoding a CAR are controlled by a single promoter and are inserted into a TRAC locus. In some embodiments, a CD4 7 transgene and a trans gene encoding a CAR are controlled by their own promoters and are inserted into a TRAC locus. In some embodiments, a CD47 transgene and a transgene encoding a CAR are inserted into a TRB locus. In some embodiments, a CD47 transgene and a transgene encoding a CAR are controlled by a single promoter and are inserted into a TRB locus. In some embodiments, a CD47 transgene and a transgene encoding a CAR are controlled by their own promoters and are inserted into a TRB locus. In other embodiments, a CD47 transgene and a transgene encoding a CAR are inserted into a B2Mlocus. In other embodiments, a CD47 transgene and a transgene encoding a CAR are controlled by a single promoter and are inserted into a B2M locus. In other embodiments, a CD47 transgene and a transgene encoding a CAR are controlled by their own promoters and are inserted into a B2M locus. In various embodiments, a CD47 transgene and a transgene encoding a CAR are inserted into a CIITA locus. In various embodiments, a CD47 transgene and a transgene encoding a CAR are controlled by a single promoter and are inserted into a CIITA locus. In various embodiments, a CD47 transgene and a transgene encoding a CAR are controlled by their own promoters and are inserted into a CIITA locus. In some instances, the promoter controlling expression of any transgene described is a constitutive promoter. In other instances, the promoter for any transgene described is an inducible promoter. In some embodiments, the promoter is an EF1 a promoter. In some embodiments, the promoter is CAG promoter. In some embodiments, a CD47 transgene and a transgene encoding a CAR are both controlled by a constitutive promoter. In some embodiments, a CD47 transgene and a transgene encoding a CAR are both controlled by an inducible promoter. In some embodiments, a CD47 transgene is controlled by a constitutive promoter and a transgene encoding a CAR is controlled by an inducible promoter. In some embodiments, a CD47 transgene is controlled by an inducible promoter and a transgene encoding a CAR is controlled by a constitutive promoter. In various embodiments, a CD47 transgene is controlled by an EF1α promoter and a transgene encoding a CAR is controlled by an EF1α promoter. In some embodiments, a CD47 transgene is controlled by a CAG promoter and a transgene encoding a CAR is controlled by a CAG promoter. In some embodiments, a CD47 transgene is controlled by a CAG promoter and a transgene encoding a CAR is controlled by an EF1a promoter. In some embodiments, a CD47 transgene is controlled by an EF1α promoter and a transgene encoding a CAR is controlled by a CAG promoter. In some embodiments, expression of both a CD47 transgene and a transgene encoding a CAR is controlled by a single EF1a promoter. In some embodiments, expression of both a CD47 transgene and a transgene encoding a CAR is controlled by a single CAG promoter.

In some embodiments, the present disclosure disclosed herein is directed to pluripotent stem cells, (e.g., pluripotent stem cells and induced pluripotent stem cells (iPSCs)), differentiated cells derived from such pluripotent stem cells (e.g., hypoimmune (HIP) T cells), and primary T cells that overexpress CD47 (such as exogenously express CD47 proteins), have reduced expression or lack expression of MHC class I and/or MHC class II human leukocyte antigens, and have reduced expression or lack expression of a T-cell receptor (TCR) complex. In some embodiments, the hypoimmune (HIP) T cells and primary T cells overexpress CD47 (such as exogenously express CD47 proteins), have reduced expression or lack expression of MHC class I and/or MHC class II human leukocyte antigens, and have reduced expression or lack expression of a T-cell receptor (TCR) complex.

In some embodiments, pluripotent stem cells, (e.g., pluripotent stem cells and induced pluripotent stem cells (iPSCs)), differentiated cells derived from such pluripotent stem cells (e.g., hypoimmune (HIP) T cells), and primary T cells overexpress CD47 and include a genomic modification of the B2M gene. In some embodiments, pluripotent stem cells, differentiated cell derived from such pluripotent stem cells and primary T cells overexpress C047 and include a genomic modification of the CIITA gene. In some embodiments, pluripotent stem cells, T cells differentiated from such pluripotent stem cells and primary T cells overexpress CD47 and include a genomic modification of the TRAC gene. In some embodiments, pluripotent stem cells, T cells differentiated from such pluripotent stem cells and primary T cells overexpress CD47 and include a genomic modification of the TRB gene. In some embodiments, pluripotent stem cells, T cells differentiated from such pluripotent stem cells and primary T cells overexpress CD47 and include one or more genomic modifications selected from the group consisting of the B2M. CIITA, TRAC and TRB genes. In some embodiments, pluripotent stem cells, T cells differentiated from such pluripotent stem cells and primary T cells overexpress CD47 and include genomic modifications of the B2M, CIITA and TRAC genes. In some embodiments, pluripotent stem cells, T cells differentiated from such pluripotent stem cells and primary T cells overexpress CD47 and include genomic modifications of the B2M, CIITA and TRB genes. In some embodiments. pluripotent stem cells, T cells differentiated from such pluripotent stem cells and primary T cells overexpress CD47 and include genomic modifications of the B2M, CIITA, TRAC and TRB genes. In some embodiments, the pluripotent stem cells, differentiated cell derived from such pluripotent stem cells and primary T cells are B2M^{-/-}, CIITA^{-/-}, TRAC^{-/-}, CD47tg cells. In some embodiments, the cells are B2M^{-/-}, CIITA^{-/-}, TRB^{-/-}, CD47tg cells. In some embodiments, the cells are B2M^{-/-}, CIITA^{-/-}, TRAC^{-/-}, TRB^{-/-}, CD47tg cells. In some embodiments, the cells are B2M^{-/-}, CIITA^{-/-}, TRAC^{-/-}, CD47tg cells. In some embodiments, the cells are B2M^{-/-}, CIITA^{-/-}, TRB^{-/-}, CD47tg cells. In some embodiments, the cells are B2M^{indel/indel}, CIITA^{indel/indel}, TRAC^{indel/indel}, TRB^{indel/indel}, CD47tg cells. In some embodiments, the engineered or modified cells described are pluripotent stem cells. T cells differentiated from such pluripotent stem cells or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), yS T cells, and any other subtype of T cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material Is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, a CD47 transgene is inserted into a pre-selected locus of the cell. In some embodiments, the pre-selected locus is a safe harbor or target locus. Non-limiting examples of a safe harbor or target locus includes a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some embodiments, the pre-selected locus is the TRAC locus. In some embodiments, a CD47 transgene is inserted into a safe harbor or target locus (e.g., a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some embodiments, a CD47 transgene is inserted into the B2M locus. In some embodiments, a CD47 transgene is inserted into the B2M locus. In some embodiments, a CD47 transgene is inserted into the TRAC locus. In some embodiments, a CD47 transgene is inserted into the TRB locus. In some embodiments, the CD47 transgene is inserted into a pre-selected locus of the cell, including a safe harbor locus, via viral vector transduction/integration. In some embodiments, the vector is a self-inactivating lentiviral vector pseudotyped with a vesicular stomatitis VSV-G envelope. In some embodiments, the CD47 transgene is inserted into at least one allele of the cell using viral transduction. In some embodiments, the exogenous polynucleotide is inserted into at least one allele of the cell using a lentivirus based viral vector.

In some instances, expression of a CD47 transgene is controlled by a constitutive promoter. In other instances, expression of a CD47 transgene is controlled by an inducible promoter. In some embodiments, the promoter is an EF1alpha (EF1α) promoter. In some embodiments, the promoter a CAG promoter.

In some embodiments, the present disclosure disclosed herein is directed to pluripotent stem cells, (e.g., pluripotent stem cells and induced pluripotent stem cells (iPSCs)), T cells derived from such pluripotent stem cells (e.g., hypoimmune (HIP) T cells), and primary T cells that have reduced expression or lack expression of MHC class I and/or MHC class II human leukocyte antigens and have reduced expression or lack expression of a T-cell receptor (TCR) complex. In some embodiments, the cells have reduced or lack expression of MHC class I antigens, MHC class II antigens, and TCR complexes.

In some embodiments, pluripotent stem cells (e.g., iPSCs), differentiated cells derived from such (e.g., T cells differentiated from such), and primary T cells include a genomic modification of the 82M gene. In some embodiments, pluripotent stem cells (e.g., iPSCs), differentiated cells derived from such (e.g., T cells differentiated from such), and primary T cells include a genomic modification of the CIITA gene. In some embodiments, pluripotent stem cells (e.g., iPSCs), T cells differentiated from such, and primary T cells include a genomic modification of the TRAC gene. In some embodiments, pluripotent stem cells (e.g., iPSCs), T cells differentiated from such, and primary T cells include a genomic modification of the TRB gene. In some embodiments, pluripotent stem cells (e.g., iPSCs), T cells differentiated from such, and primary T cells include one or more genomic modifications selected from the group consisting of the B2M, CIITA and TRAC genes. In some embodiments, pluripotent stem cells (e.g., iPSCs). T cells differentiated from such, and primary T cells include one or more genomic modifications selected from the group consisting of the B2M, CIITA and TRB genes. In some embodiments, pluripotent stem cells (e.g., iPSCs), T cells differentiated from such, and primary T cells include one or more genomic modifications selected from the group consisting of the B2M, CIITA, TRAC and TRB genes. In some embodiments, the cells including iPSCs. T cells differentiated from such, and primary T cells are B2M^{-/-}, CIITA^{-/-}, TRAC^{-/-}cels. In some embodiments, the ceils including iPSCs, T cells differentiated from such, and primary T cells are B2M^{-/-}, CIITA^{-/-}, TRB^{-/-} cells. In some embodiments, the cells including iPSCs, T cells differentiated from such, and primary T cells are B2M^{indel/indel}, CIITA^{indel/indel}, TRAC^{indel/indel}cells. In some embodiments, the cells including iPSCs, T cells differentiated from such, and primary T cells are B2M^{indel/indel}, CIITA^{indel/indel}, TRB^{indel/indel}cells. In some embodiments, the cells including iPSCs, T cells differentiated from such, and primary T cells are B2M^{indel/indel}, CIITA^{indel/indel}, TRAC^{indel/indel}, TRB^{indel/indel}cells. In some embodiments, the modified cells described are pluripotent stem cells, induced pluripotent stem cells, T cells differentiated from such pluripotent stem cells and induced pluripotent stem cells, or primary T cells. Non-limiting examples of primary T cells include CD3+ T cells, CD4+ T cells, CD8+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

Cells of the present disclosure exhibit reduced or lack expression of MHC class I antigens, MHC class II antigens, and/or TCR complexes. In some embodiments, reduction of MHC I and/or MHC II expression is accomplished, for example, by one or more of the following: (1) targeting the polymorphic HLA alleles (HLA-A, HLA-B, HLA-C) and MHC-II genes directly; (2) removal of B2M, which will prevent surface trafficking of all MHC-I molecules; (3) removal of CIITA, which will prevent surface trafficking of all MHC-II molecules; and/or (4) deletion of components of the MHC enhanceosomes, such as LRC5, RFX5, RFXANK, RFXAP, IRFI, NF-Y (including NFY-A, NFY—B, NFY-C), and CIITA that are critical for HLA expression.

In some embodiments, HLA expression is interfered with by targeting individual HIAs (e.g., knocking out, knocking down, or reducing expression of HLA-A, HLA-B, HLA-C. HLA-DP. HLA-DO, and/or HLA-DR), targeting transcriptional regulators of HLA expression (e.g., knocking out, knocking down, or reducing expression of NLRC5, CIITA, RFX5, RFXAP, RFXANK, NFY-A, NFY—B, NFY—C and/or IRF-1), blocking surface trafficking of MHC class I molecules (e.g., knocking out, knocking down, or reducing expression of B2M and/or TAP1), and/or targeting with HLA-Razor (see, e.g., WO2016183041).

In some embodiments, the cells disclosed herein including, but not limited to, pluripotent stem cells, induced pluripotent stem cells, differentiated cells derived from such stem cells, and primary T cells do not express one or more human leukocyte antigens (e.g., HLA-A, HLA-B, HLA-C, HLA-DP, HLA-DQ, and/or HLA-DR) corresponding to MHC-I and/or MHC-II and are thus characterized as being hypoimmunogenic. For example, in some embodiments, the pluripotent stem cells and induced pluripotent stem cells disclosed have been modified such that the stem cell or a differentiated stem cell prepared therefrom do not express or exhibit reduced expression of one or more of the following MHC-4 molecules: HLA-A. HLA-B and HLA-C. In some embodiments, one or more of HLA-A, HLA-B and HLA-C is “knocked-out” of a cell. A cell that has a knocked-out HLA-A gene, HLA-B gene, and/or HLA-C gene may exhibit reduced or eliminated expression of each knocked-out gene.

In some embodiments, guide RNAs, shRNAs, siRNAs, or miRNAs that allow simultaneous deletion of all MHC class I alleles by targeting a conserved region in the HLA genes are identified as HLA Razors. In some embodiments, the gRNAs are part of a CRISPR system. In alternative embodiments, the gRNAs are part of a TALEN system. In some embodiments, an HLA Razor targeting an identified conserved region in HLAs is described in WO2016183041. In some embodiments, multiple HLA Razors targeting identified conserved regions are utilized. It is generally understood that any guide, siRNA, shRNA, or miRNA molecule that targets a conserved region in HLAs can act as an HLA Razor.

Methods provided are useful for inactivation or ablation of MHC class I expression and/or MHC class II expression in cells such as but not limited to pluripotent stem cells, differentiated cells, and primary T cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of genes involved in an immune response (e.g., by deleting genomic DNA of genes involved in an immune response or by insertions of genomic DNA into such genes, such that gene expression is impacted) in cells. In some embodiments, genome editing technologies or other gene modulation technologies are used to insert tolerance-inducing factors in human cells, rendering them and the differentiated cells prepared therefrom hypoimmunogenic cells. As such, the hypoimmunogenic cells have reduced or eliminated expression of MHC I and MHC II expression. In some embodiments, the cells are nonimmunogenic (e.g., do not induce an innate and/or an adaptive immune response) in a recipient subject.

In some embodiments, the cell includes a modification to increase expression of CD47 and one or more factors selected from the group consisting of DUX4, CD24, CD27, CD35, CD46, CD55, CD59, CD200, HLA-C, HLA-E, HLA-E heavy chain, HLA-G, PD-L1, IDO1, CTLA4-Ig. C1-Inhibitor, IL-10, IL-35, IL-39, FasL, CCL21, CCL22, Mfge8, CD16, CD52, H2-M3, CD16 Fc receptor, IL15-RF, H2-M3(HLA-G), B2M-HLA-E, A20ITNFAIP3, CR1, HLA-F, MANF, and/or Serpinb9.

In some embodiments, the cell comprises a genomic modification of one or more target polynucleotide sequences that regulate the expression of either MHC class I molecules, MHC class II molecules, or MHC class I and MHC class II molecules. In some embodiments, a genetic editing system is used to modify one or more target polynucleotide sequences. In some embodiments, the targeted polynucleotide sequence is one or more selected from the group including 82M, CIITA, and NLRC5. In some embodiments, the cell comprises a genetic editing modification to the B2M gene. In some embodiments, the cell comprises a genetic editing modification to the CIITA gene. In some embodiments, the cell comprises a genetic editing modification to the NLRC5 gene. In some embodiments, the cell comprises genetic editing modifications to the B2M and CIITA genes. In some embodiments, the cell comprises genetic editing modifications to the B2M and NLRC5 genes. In some embodiments, the cell comprises genetic editing modifications to the CIITA and NLRC5 genes. In numerous embodiments, the cell comprises genetic editing modifications to the B2M. CIITA and NLRC5 genes. In some embodiments, the genome of the cell has been altered to reduce or delete critical components of HLA expression. In some embodiments, the cells are modified or engineered as compared to a wild-type or control cell, including an unaltered or unmodified wild-type cell or control cell. In some embodiments, the wiki-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the present disclosure provides a cell (e.g., stem cell, induced pluripotent stem cell, differentiated cell such as a primary NK cell, CAR-NK cell, primary T cell or CAR-T cell) or population thereof comprising a genome in which a gene has been edited to delete a contiguous stretch of genomic DNA, thereby reducing or eliminating surface expression of MHC class I molecules in the cell or population thereof. In some embodiments, the present disclosure provides a cell (e.g., stem cell, induced pluripotent stem cell, differentiated cell such as a primary NK cell, CAR-NK cell, primary T cell or CAR-T cell) or population thereof comprising a genome in which a gene has been edited to delete a contiguous stretch of genomic DNA, thereby reducing or eliminating surface expression of MHC class II molecules in the cell or population thereof. In numerous embodiments, the present disclosure provides a cell (e.g., stem cell, induced pluripotent stem cell, differentiated cell, hematopoietic stem cell, primary T cell or CAR-T cell) or population thereof comprising a genome in which one or more genes has been edited to delete a contiguous stretch of genomic DNA, thereby reducing or eliminating surface expression of MHC class I and II molecules in the cell or population thereof.

In some embodiments, the expression of MHC I molecules and/or MHC 11 molecules is modulated by targeting and deleting a contiguous stretch of genomic DNA, thereby reducing or eliminating expression of a target gene selected from the group consisting of B2M, CIITA, and NLRC5. In some embodiments, described herein are genetically edited cells (e.g., modified human cells) comprising exogenous CD47 proteins and inactivated or modified CIITA gene sequences, and in some instances, additional gene modifications that inactivate or modify 82M gene sequences. In some embodiments, described herein are genetically edited cells comprising exogenous CD47 proteins and inactivated or modified CIITA gene sequences, and in some instances, additional gene modifications that inactivate or modify NLRC5 gene sequences. In some embodiments, described herein are genetically edited cells comprising exogenous C047 proteins and inactivated or modified B2M gene sequences, and in some instances, additional gene modifications that inactivate or modify NLRC5 gene sequences. In some embodiments, described herein are genetically edited cells comprising exogenous CD47 proteins and inactivated or modified B2M gene sequences, and in some instances, additional gene modifications that inactivate or modify CIITA gene sequences and NLRC5 gene sequences.

Provided herein are cells exhibiting a modification of one or more targeted polynucleotide sequences that regulates the expression of any one of the following: (a) MHC I antigens, (b) MHC 11 antigens, (c) TCR complexes, (d) both MHC I and |1 antigens, and (e) MHC I and II antigens and TCR complexes. In some embodiments, the modification includes increasing expression of CD47. In some embodiments, the cells include an exogenous or recombinant CD47 polypeptide. In some embodiments, the modification includes expression of a chimeric antigen receptor. In some embodiments, the cells comprise an exogenous or recombinant chimeric antigen receptor polypeptide.

In some embodiments, the cell includes a genomic modification of one or more targeted polynucleotide sequences that regulates the expression of MHC I antigens, MHC II antigens and/or TCR complexes. In some embodiments, a genetic editing system is used to modify one or more targeted polynucleotide sequences. In some embodiments, the polynucleotide sequence targets one or more genes selected from the group consisting of B2M, CIITA, TRAC, and TRB. In some embodiments, the genome of a T cell (e.g., a T cell differentiated from hypoimmunogenic iPSCs and a primary T cell) has been altered to reduce or delete critical components of HLA and TCR expression, e.g., HLA-A antigen, HLA-B antigen, HLA-C antigen, HLA-DP antigen. HLA-DO antigen, HLA-DR antigens, TCR-alpha and TCR-beta.

In some embodiments, the present disclosure provides a cell or population thereof comprising a genome in which a gene has been edited to delete a contiguous stretch of genomic DNA, thereby reducing or eliminating surface expression of MHC class I molecules in the cell or population thereof. In some embodiments, the present disclosure provides a cell or population thereof comprising a genome in which a gene has been edited to delete a contiguous stretch of genomic DNA, thereby reducing or eliminating surface expression of MHC class II molecules in the cell or population thereof. In some embodiments, the present disclosure provides a cell or population thereof comprising a genome in which a gene has been edited to delete a contiguous stretch of genomic DNA, thereby reducing or eliminating surface expression of TCR molecules in the cell or population thereof. In numerous embodiments, the present disclosure provides a cell or population thereof comprising a genome in which one or more genes has been edited to delete a contiguous stretch of genomic DNA, thereby reducing or eliminating surface expression of MHC class I and II molecules and TCR complex molecules in the cell or population thereof.

In some embodiments, the cells and methods described herein include genomically editing human cells to cleave CIITA gene sequences as well as editing the genome of such cells to alter one or more additional target polynucleotide sequences such as, but not limited to, B2M TRAC, and TRB. In some embodiments, the cells and methods described herein include genomically editing human cells to cleave B2M gene sequences as well as editing the genome of such cells to alter one or more additional target polynucleotide sequences such as, but not limited to, CIITA, TRAC, and TRB. In some embodiments, the cells and methods described herein include genomically editing human cells to cleave TRAC gene sequences as well as editing the genome of such cells to alter one or more additional target polynucleotide sequences such as, but not limited to, B2M, CIITA, and TRB. In some embodiments, the cells and methods described herein include genomically editing human cells to cleave TRB gene sequences as well as editing the genome of such cells to alter one or more additional target polynucleotide sequences such as, but not limited to, B2M, CIITA, and TRAC.

Provided herein are hypoimmunogenic stem cells comprising reduced expression of HLA-A, HLA-B, HLA-C, HLA-DP, HLA-DQ, HLA-DR, B2M, CIITA, TCR-alpha, and TCR-beta relative to a wild-type stem cell, the hypoimmunogenic stem cell further comprising a set of exogenous polynucleotides comprising a first exogenous polynucleotide encoding CD47 and a second exogenous polynucleotide encoding a chimeric antigen receptor (CAR) as disclosed herein, wherein the first and/or second exogenous polynucleotides are inserted into a specific locus of at least one allele of the cell. Also provided herein are hypoimmunogenic primary T cells including any subtype of primary T cells comprising reduced expression of HLA-A, HLA-B, HLA-C, HLA-DP, HLA-DQ, HLA-DR, B2M, CIITA, TCR-alpha, and TCR-beta relative to a wild-type primary T cell, the hypoimmunogenic stem cell further comprising a set of exogenous polynucleotides comprising a first exogenous polynucleotide encoding CD47 and a second exogenous polynucleotide encoding a chimeric antigen receptor (CAR) as disclosed herein, wherein the first and/or second exogenous polynucleotides are inserted into a specific locus of at least one allele of the cell. Further provided herein are hypoimmunogenic T cells differentiated from hypoimmunogenic induced pluripotent stem cells comprising reduced expression of HLA-A, HLA-B, HLA-C, HLA-DP, HLA-DQ, HLA-DR, B2M, CIITA, TCR-alpha, and TCR-beta relative to a wild-type primary T cell, the hypoimmunogenic stem cell further comprising a set of exogenous polynucleotides comprising a first exogenous polynucleotide encoding CD47 and a second exogenous polynucleotide encoding a chimeric antigen receptor (CAR) as disclosed herein, wherein the first and/or second exogenous polynucleotides are inserted into a specific locus of at least one allele of the cell.

In some embodiments, the population of engineered cells described evades NK cell mediated cytotoxicity upon administration to a recipient patient. In some embodiments, the population of engineered cells evades NK cell mediated cytotoxicity by one or more subpopulations of NK cells. In some embodiments, the population of engineered is protected from cell lysis by NK cells, including immature and/or mature NK cells upon administration to a recipient patient. In some embodiments, the population of engineered cells evades macrophage engulfment upon administration to a recipient patient. In some embodiments, the population of engineered cells does not induce an innate and/or an adaptive immune response to the cell upon administration to a recipient patient.

In some embodiments, the cells described herein comprise a safety switch. The term “safety switch” used herein refers to a system for controlling the expression of a gene or protein of interest that, when downregulated or upregulated, leads to clearance or death of the cell, e.g., through recognition by the host's immune system. A safety switch is designed to be triggered by an exogenous molecule in case of an adverse clinical event. A safety switch is engineered by regulating the expression on the DNA, RNA and protein levels. A safety switch includes a protein or molecule that allows for the control of cellular activity in response to an adverse event. In one embodiment, the safety switch is a “kill switch” that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis. In some embodiments, the cells described herein, e.g., stem cells, induced pluripotent stem cells, hematopoietic stem cells, primary cells, or differentiated cell, including, but not limited to, T cells, CAR-T cells, NK cells, and/or CAR-NK cells, comprise a safety switch.

In some embodiments, the safety switch comprises a therapeutic agent that inhibits or blocks the interaction of CD47 and SIRPα. In some aspects, the CD47-SIRPα blockade agent is an agent that neutralizes, blocks, antagonizes, or interferes with the cell surface expression of CD47, SIRPα, or both. In some embodiments, the CD47-SIRPα blockade agent inhibits or blocks the interaction of CD47, SIRPα or both. In some embodiments, a CD47-SIRPα blockade agent (e.g., a CD47-SIRPα blocking, inhibiting, reducing, antagonizing, neutralizing, or interfering agent) comprises an agent selected from a group that includes an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

In some embodiments, the cells described herein comprise a “suicide gene” (or “suicide switch”). The suicide gene can cause the death of the hypoimmunogenic cells should they grow and divide in an undesired manner. The suicide gene ablation approach includes a suicide gene in a gene transfer vector encoding a protein that results in cell killing only when activated by a specific compound. A suicide gene can encode an enzyme that selectively converts a nontoxic compound into highly toxic metabolites. In some embodiments, the cells described herein, e.g., stem cells, induced pluripotent stem cells, hematopoietic stem cells, primary cells, or differentiated cell, including, but not limited to, T cells, CAR-T cells, NK cells, and/or CAR-NK cells, comprise a suicide gene.

In some embodiments, the population of engineered cells described elicits a reduced level of immune activation or no immune activation upon administration to a recipient subject. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicit a reduced level of IgM and IgG antibody production or no IgM and IgG antibody production against the cells in a recipient subject. In some embodiments, the cells elicit a reduced level of cytotoxic T cell killing of the cells upon administration to a recipient subject.

### A. CIITA

In some embodiments, the technologies disclosed herein modulate (e.g., reduces or eliminates) the expression of MHC 11 genes by targeting and modulating (e.g., reducing or eliminating) Class 11 transactivator (CIITA) expression. In some embodiments, the modulation occurs using a CRISPR/Cas system. CIITA is a member of the LR or nucleotide binding domain (NBD) leucine-rich repeat (LRR) family of proteins and regulates the transcription of MHC 11 by associating with the MHC enhanceosome.

In some embodiments, the target polynucleotide sequence of the present disclosure is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the cells described herein comprise gene modifications at the gene locus encoding the CIITA protein. In other words, the cells comprise a genetic modification at the CIITA locus. In some instances, the nucleotide sequence encoding the CIITA protein is set forth in RefSeq. No. NM_000246.4 and NCBI Genbank No. U18259. In some instances, the CIITA gene locus is described in NCBI Gene ID No. 4261. In some embodiments, the amino acid sequence of CIITA is depicted as NCBI GenBank No. AAA88861.1. Additional descriptions of the CIITA protein and gene locus can be found in Uniprot No. P33076, HGNC Ref. No. 7067, and OMIM Ref. No. 600005.

In some embodiments, the hypoimmunogenic cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOs:5184-36352 of Table 12 of WO2016183041, which is herein incorporated by reference. In some embodiments, the cell has a reduced ability to induce an innate and/or an adaptive immune response in a recipient subject. In some embodiments, an exogenous nucleic acid encoding a polypeptide as disclosed herein (e.g., a chimeric antigen receptor, CD47, or another tolerogenic factor disclosed herein) is inserted at the CIITA gene.

Assays to test whether the CIITA gene has been inactivated are known and described herein. In some embodiments, the resulting genetic modification of the CIITA gene by PCR and the reduction of HLA-II expression is assayed by FACS analysis. In some embodiments, CIITA protein expression is detected using a Western blot of cells lysates probed with antibodies to the CIITA protein. In some embodiments, reverse transcriptase polymerase chain reactions (RT-PCR) are used to confirm the presence of the inactivating genetic modification. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

### B. B2M

In some embodiments, the technologies disclosed herein modulate (e.g., reduce or eliminate) the expression of MHC-I genes by targeting and modulating (e.g., reducing or eliminating) expression of the accessory chain B2M. In some embodiments, the modulation occurs using a CRISPR/Cas system. By modulating (e.g., reducing or deleting) expression of B2M, surface trafficking of MHC-I molecules is blocked and the cell rendered hypoimmunogenic. In some embodiments, the cell has a reduced ability to induce an innate and/or an adaptive immune response in a recipient subject.

In some embodiments, the target polynucleotide sequence of the present disclosure is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC I molecules: HLA-A, HLA-B, and HLA-C.

In some embodiments, the cells described herein comprise gene modifications at the gene locus encoding the B2M protein. In other words, the cells comprise a genetic modification at the 82M locus. In some instances, the nucleotide sequence encoding the B2M protein is set forth in RefSeq. No. NM_004048.4 and Genbank No. A8021288.1. In some instances, the B2M gene locus is described in NCBI Gene ID No. 567. In some embodiments, the amino acid sequence of B2M is depicted as NCBI GenBank No. BAA35182.1. Additional descriptions of the 82M protein and gene locus can be found in Uniprot No. P61769, HGNC Ref. No. 914, and OMIM Ref. No. 109700.

In some embodiments, the hypoimmunogenic cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOs:81240-85644 of Table 15 of WO2016183041, which is herein incorporated by reference. In some embodiments, an exogenous nucleic acid encoding a polypeptide as disclosed herein (e.g., a chimeric antigen receptor, CD47, or another tolerogenic factor disclosed herein) is inserted at the B2M gene. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

Assays to test whether the 82M gene has been inactivated are known and described herein. In some embodiments, the resulting genetic modification of the 82M gene by PCR and the reduction of HLA-I expression is assayed by FACS analysis. In some embodiments, B2M protein expression is detected using a Western blot of cells lysates probed with antibodies to the 82M protein. In some embodiments, reverse transciptase polymerase chain reactions (RT-PCR) are used to confirm the presence of the inactivating genetic modification.

### C. NLRC5

In many embodiments, the technologies disclosed herein modulate (e.g., reduce or eliminate) the expression of MHC-I genes by targeting and modulating (e.g., reducing or eliminating) expression of the NLR family, CARD domain containing 5/NOD27/CLR16.1 (NLRC5). In some embodiments, the modulation occurs using a CRISPR/Cas system. NLRC5 is a critical regulator of MHC-1-mediated immune responses and, similar to CIITA, NLRC5 is highly inducible by IFN-γ and can translocate into the nucleus. NLRC5 activates the promoters of MHC-I genes and induces the transcription of MHC-I as well as related genes involved in MHC-4 antigen presentation.

In some embodiments, the target polynucleotide sequence is a variant of NLRC5. In some embodiments, the target polynucleotide sequence is a homolog of NLRC5. In some embodiments, the target polynucleotide sequence is an ortholog of NLRC5.

In some embodiments, decreased or eliminated expression of NLRC5 reduces or eliminates expression of one or more of the following MHC I molecules—HLA-A, HLA-B, and HLA-C.

In some embodiments, the cells outlined herein comprise a genetic modification targeting the NLRC5 gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene. In some embodiments, the at least one guide ribonucleic acid sequence for specifically targeting the NLRC5 gene is selected from the group consisting of SEQ ID NOs:36353-81239 of Appendix 3 or Table 14 of WO2016183041, the disclosure is incorporated by reference in its entirety.

Assays to test whether the NLRC5 gene has been inactivated are known and described herein. In some embodiments, the resulting genetic modification of the NLRC5 gene by PCR and the reduction of HLA-I expression is assayed by FACS analysis. In some embodiments, NLRC5 protein expression is detected using a Western blot of cells lysates probed with antibodies to the NLRC5 protein. In some embodiments, reverse transcriptase polymerase chain reactions (RT-PCR) are used to confirm the presence of the inactivating genetic modification.

### D. TRAC

In many embodiments, the technologies disclosed herein modulate (e.g., reduce or eliminate) the expression of TCR genes including the TRAC gene by targeting and modulating (e.g., reducing or eliminating) expression of the constant region of the T cell receptor alpha chain. In some embodiments, the modulation occurs using a CRISPR/Cas system. By modulating (e.g., reducing or deleting) expression of TRAC, surface trafficking of TCR molecules is blocked. In some embodiments, the is cell also has a reduced ability to induce an innate and/or an adaptive immune response in a recipient subject.

In some embodiments, the target polynucleotide sequence of the present disclosure is a variant of TRAC. In some embodiments, the target polynucleotide sequence is a homolog of TRAC. In some embodiments, the target polynucleotide sequence is an ortholog of TRAC.

In some embodiments, decreased or eliminated expression of TRAC reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRAC protein. In other words, the cells comprise a genetic modification at the TRAC locus. In some instances, the nucleotide sequence encoding the TRAC protein is set forth in Genbank No. X02592.1. In some Instances, the TRAC gene locus is described in RefSeq. No. NG_001332.3 and NCBI Gene ID No. 28755. In some embodiments, the amino acid sequence of TRAC is depicted as Uniprot No. P01848. Additional descriptions of the TRAC protein and gene locus can be found in Uniprot No. P01848, HGNC Ref. No. 12029, and OMIM Ref. No. 186880.

In some embodiments, the hypoimmunogenic cells outlined herein comprise a genetic modification targeting the TRAC gene. In some embodiments, the genetic modification targeting the TRAC gene by the rare-cutting endonuclease comprises a Cas protein or a polynucleotide encoding a Cas protein, and at least one guide ribonucleic acid sequence for specifically targeting the TRAC gene. In some embodiments, the at least one guide ribonucleic acid sequence for specifically targeting the TRAC gene is selected from the group consisting of SEQ ID NOs:532-609 and 9102-9797 of US20160348073, which is herein incorporated by reference.

Assays to test whether the TRAC gene has been inactivated are known and described herein. In some embodiments, the resulting genetic modification of the TRAC gene by PCR and the reduction of TCR expression is assayed by FACS analysis. In some embodiments, TRAC protein expression is detected using a Western blot of cells lysates probed with antibodies to the TRAC protein. In some embodiments, reverse transcriptase polymerase chain reactions (RT-PCR) are used to confirm the presence of the inactivating genetic modification.

### E. TRB

In many embodiments, the technologies disclosed herein modulate (e.g., reduce or eliminate) the expression of TCR genes including the gene encoding T cell antigen receptor, beta chain (e.g., the TRB, TRBC, or TCRB gene) by targeting and modulating (e.g., reducing or eliminating) expression of the constant region of the T cell receptor beta chain. In some embodiments, the modulation occurs using a CRISPR/Cas system. By modulating (e.g., reducing or deleting) expression of TRB, surface trafficking of TCR molecules is blocked. In some embodiments, the cell also has a reduced ability to induce an innate and/or an adaptive immune response in a recipient subject.

In some embodiments, the target polynucleotide sequence of the present disclosure is a variant of TRB. In some embodiments, the target polynucleotide sequence is a homolog of TRB. In some embodiments, the target polynucleotide sequence is an ortholog of TRB.

In some embodiments, decreased or eliminated expression of TRB reduces or eliminates TCR surface expression.

In some embodiments, the cells, such as, but not limited to, pluripotent stem cells, induced pluripotent stem cells. T cells differentiated from induced pluripotent stem cells, primary T cells, and cells derived from primary T cells comprise gene modifications at the gene locus encoding the TRB protein. In other words, the cells comprise a genetic modification at the TRB gene locus. In some instances, the nucleotide sequence encoding the TRB protein is set forth in UniProt No. PODSE2. In some instances, the TRB gene locus is described in RefSeq. No. NG,_001333.2 and NCBI Gene ID No. 6957. In some embodiments, the amino acid sequence of TRB is depicted as Uniprot No. P01848. Additional descriptions of the TRB protein and gene locus can be found in GenBank No. L36092.2, Uniprot No. PODSE2, and HGNC Ref. No. 12155.

In some embodiments, the hypoimmunogenic cells outlined herein comprise a genetic modification targeting the TRB gene. In some embodiments, the genetic modification targeting the TRB gene by the rare-cutting endonuclease comprises a Cas protein or a polynucleotide encoding a Cas protein, and at least one guide ribonucleic acid sequence for specifically targeting the TRB gene. In some embodiments, the at least one guide ribonucleic acid sequence for specifically targeting the TRB gene is selected from the group consisting of SEQ ID NOs:610-765 and 9798-10532 of US20160348073, which is herein incorporated by reference.

Assays to test whether the TRB gene has been inactivated are known and described herein. In some embodiments, the resulting genetic modification of the TRB gene by PCR and the reduction of TCR expression is assayed by FACS analysis. In some embodiments, TRB protein expression is detected using a Western blot of cells lysates probed with antibodies to the TRB protein. In some embodiments, reverse transcriptase polymerase chain reactions (RT-PCR) are used to confirm the presence of the inactivating genetic modification.

### F. CD142

In many embodiments, the technologies disclosed herein modulate (e.g., reduce or eliminate) the expression of CD142, which is also known as tissue factor, factor Ill, and F3. In some embodiments, the modulation occurs using a gene editing system (e.g., CRISPR/Cas).

In some embodiments, the target polynucleotide sequence is CD142 or a variant of CD142. In some embodiments, the target polynucleotide sequence is a homolog of CD142. In some embodiments, the target polynucleotide sequence is an ortholog of CD142.

In some embodiments, the cells outlined herein comprise a genetic modification targeting the CD142 gene. In some embodiments, the genetic modification targeting the CD142 gene by the rare-cutting endonuclease comprises a Cas protein or a polynucleotide encoding a Cas protein, and at least one guide ribonucleic acid (gRNA) sequence for specifically targeting the CD142 gene. Useful methods for identifying gRNA sequences to target CD142 are described below.

Assays to test whether the CD142 gene has been inactivated are known and described herein. In some embodiments, the resulting genetic modification of the CD142 gene by PCR and the reduction of CD142 expression is assayed by FACS analysis. In some embodiments, CD142 protein expression is detected using a Western blot of cells lysates probed with antibodies to the CD142 protein. In some embodiments, reverse transcriptase polymerase chain reactions (RT-PCR) are used to confirm the presence of the inactivating genetic modification.

Useful genomic, polynucleotide and polypeptide information about the human CD142 are provided in, for example, the GeneCard Identifier GC01M094530, HGNC No. 3541, NCBI Gene ID 2152, NCBI RefSeq Nos. NM_001178096.1, NM_001993.4, NP_001171567.1, and NP_001984.1, UniProt No. P13726, and the like.

### G. CTLA-4

In some embodiments, the target polynucleotide sequence is CTLA-4 or a variant of CTLA-4. In some embodiments, the target polynucleotide sequence is a homolog of CTLA-4. In some embodiments, the target polynucleotide sequence is an ortholog of CTLA-4.

In some embodiments, the cells outlined herein comprise a genetic modification targeting the CTLA-4 gene. In some embodiments, primary T cells comprise a genetic modification targeting the CTLA-4 gene. The genetic modification can reduce expression of CTLA-4 polynucleotides and CTLA-4 polypeptides in T cells includes primary T cells and CAR-T cells. In some embodiments, the genetic modification targeting the CTLA-4 gene by the rare-cutting endonuclease comprises a Cas protein or a polynucleotide encoding a Cas protein, and at least one guide ribonucleic acid (gRNA) sequence for specifically targeting the CTLA-4 gene. Useful methods for identifying gRNA sequences to target CTLA-4 are described below.

Assays to test whether the CTLA-4 gene has been inactivated are known and described herein. In some embodiments, the resulting genetic modification of the CTLA-4 gene by PCR and the reduction of CTLA-4 expression is assayed by FACS analysis. In some embodiments, CTLA-4 protein expression is detected using a Western blot of cells lysates probed with antibodies to the CTLA-4 protein. In some embodiments, reverse transcriptase polymerase chain reactions (RT-PCR) are used to confirm the presence of the inactivating genetic modification.

Useful genomic, polynucleotide and polypeptide information about the human CTLA-4 are provided in, for example, the GeneCard Identifier GC02P203867, HGNC No. 2505, NCBI Gene ID 1493, NCBI RefSeq Nos. NM_005214.4, NM_001037631.2, NP_001032720.1 and NP 005205.2, UniProt No. P16410, and the like.

### H. PD-1

In some embodiments, the target polynucleotide sequence is PD-1 ora variant of PD-1. In some embodiments, the target polynucleotide sequence is a homolog of PD-1. In some embodiments, the target polynucleotide sequence is an ortholog of PD-1.

In some embodiments, the cells outlined herein comprise a genetic modification targeting the gene encoding the programmed cell death protein 1 (PD-1) protein or the PDCD1 gene. In some embodiments, primary T cells comprise a genetic modification targeting the PDCD1 gene. The genetic modification can reduce expression of PD-1 polynucleotides and PD-1 polypeptides in T cells includes primary T cells and CAR-T cells. In some embodiments, the genetic modification targeting the PDCD1 gene by the rare-cutting endonuclease comprises a Cas protein or a polynucleotide encoding a Cas protein, and at least one guide ribonucleic acid (gRNA) sequence for specifically targeting the PDCD1 gene. Useful methods for identifying gRNA sequences to target PD-1 are described below.

Assays to test whether the PDCD1 gene has been inactivated are known and described herein. In some embodiments, the resulting genetic modification of the PDCD1 gene by PCR and the reduction of PD-1 expression is assayed by FACS analysis. In some embodiments, PD-1 protein expression is detected using a Western blot of cells lysates probed with antibodies to the PD-1 protein. In some embodiments, reverse transcriptase polymerase chain reactions (RT-PCR) are used to confirm the presence of the inactivating genetic modification.

Useful genomic, polynucleotide and polypeptide information about human PD-1 including the PDCD1 gene are provided in, for example, the GeneCard Identifier GC02M241849, HGNC No. 8760, NCBI Gene ID 5133, Uniprot No. Q15116, and NCBI RefSeq Nos. NM_005018.2 and NP_005009.2.

### I. CD47

In some embodiments, the present disclosure provides a cell or population thereof that has been modified to express the tolerogenic factor (e.g., immunomodulatory polypeptide) CD47. In some embodiments, the present disclosure provides a method for altering a cell genome to express CD47. In some embodiments, the stem cell expresses exogenous CD47. In some instances, the cell expresses an expression vector comprising a nucleotide sequence encoding a human CD47 polypeptide. In some embodiments, the cell is genetically modified to comprise an integrated exogenous polynucleotide encoding CD47 using homology-directed repair. In some instances, the cell expresses a nucleotide sequence encoding a human CD47 polypeptide such that the nucleotide sequence is inserted into at least one allele of a safe harbor or target locus. In some instances, the cell expresses a nucleotide sequence encoding a human CD47 polypeptide wherein the nucleotide sequence is inserted into at least one allele of an AAVS1 locus. In some instances, the cell expresses a nucleotide sequence encoding a human CD47 polypeptide wherein the nucleotide sequence is inserted into at least one allele of a CCR5 locus. In some instances, the cell expresses a nucleotide sequence encoding a human CD47 polypeptide wherein the nucleotide sequence is inserted into at least one allele of a safe harbor or target gene locus, such as, but not limited to, a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some instances, the cell expresses a nucleotide sequence encoding a human CD47 polypeptide wherein the nucleotide sequence is inserted into at least one allele of a TRAC locus.

CD47 is a leukocyte surface antigen and has a role in cell adhesion and modulation of integrins. It is expressed on the surface of a cell and signals to circulating macrophages not to eat the cell.

In some embodiments, the cell outlined herein comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a nucleotide sequence encoding a CD47 polypeptide having an amino acid sequence as set forth in NCBI Ref. Sequence Nos. NP 001768.1 and NP 942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises a nucleotide sequence for CD47 as set forth in NCBI Ref. Sequence Nos. NM_001777.3 and NM_198793.2. In some embodiments, the nucleotide sequence encoding a CD47 polynucleotide is a codon optimized sequence. In some embodiments, the nucleotide sequence encoding a CD47 polynucleotide is a human codon optimized sequence.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a CD47 polypeptide having an amino acid sequence as set forth in NCBI Ref. Sequence Nos. NP_001768.1 and NP_942088.1.

Exemplary amino acid sequences of human CD47 with a signal sequence and without a signal sequence are provided in Table 27.

In some embodiments, the cell comprises a CD47 polypeptide having at least 95% sequence identity (e.g., 95%, 96%, 97%, 98%, 99%, or more) to the amino acid sequence of SEQ ID NO:167. In some embodiments, the cell comprises a CD47 polypeptide having the amino acid sequence of SEQ ID NO:1167. In some embodiments, the cell comprises a CD47 polypeptide having at least 95% sequence identity (e.g., 95%, 96%, 97%, 98%, 99%, or more) to the amino acid sequence of SEQ ID NO:188. In some embodiments, the cell comprises a CD47 polypeptide having the amino acid sequence of SEQ ID NO:168.

In some embodiments, the cell comprises a nucleotide sequence encoding a CD47 polypeptide having at least 95% sequence identity (e.g., 95%, 96%, 97%, 98%, 99%. or more) to the amino acid sequence of SEQ ID NO:167. In some embodiments, the cell comprises a nucleotide sequence encoding a CD47 polypeptide having the amino acid sequence of SEQ ID NO:167. In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO:168. In some embodiments, the cell comprises a nucleotide sequence encoding a CD47 polypeptide having the amino acid sequence of SEQ ID NO:168. In some embodiments, the nucleotide sequence is codon optimized for expression in a particular cell.

In some embodiments, a suitable gene editing system (e.g., CRISPR/Cas system or any of the gene editing systems described herein) is used to facilitate the insertion of a polynucleotide encoding CD47, into a genomic locus of the hypoimmunogenic cell. In some embodiments, the polynucleotide encoding CD47 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD47 is inserted into a B2M gene locus, a CIITA gene locus, a TRAC gene locus, or a TRB gene locus. In some embodiments, the polynucleotide encoding CD47 is operably linked to a promoter.

In some embodiments, the polynucleotide encoding CD47 is inserted into at least one allele of the T cell using viral transduction. In some embodiments, the polynucleotide encoding CD47 is inserted into at least one allele of the T cell using a lentivirus based viral vector. In some embodiments, the lentivirus based viral vector is a pseudotyped, self-inactivating lentiviral vector that carries the polynucleotide encoding CD47. In some embodiments, the lentivirus based viral vector is a self-inactivating lentiviral vector pseudotyped with a vesicular stomatitis VSV-G envelope, and which carries the polynucleotide encoding CD47.

In some embodiments, CD47 protein expression is detected using a Western blot of cell lysates probed with antibodies against the CD47 protein. In some embodiments, reverse transcriptase polymerase chain reactions (RT-PCR) are used to confirm the presence of the exogenous CD47 mRNA.

### J. CD24

In some embodiments, the present disclosure provides a cell or population thereof that has been modified to express the tolerogenic factor (e.g., immunomodulatory polypeptide) CD24. In some embodiments, the present disclosure provides a method for altering a cell genome to express CD24. In some embodiments, the stem cell expresses exogenous CD24. In some instances, the cell expresses an expression vector comprising a nucleotide sequence encoding a human CD24 polypeptide. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

CD24 which is also referred to as a heat stable antigen or small-cell lung cancer cluster 4 antigen is a glycosylated glycosylphosphatidylinositol-anchored surface protein (Pirruccello et al., J Immunol, 1986, 136, 3779-3784; Chen et al., Glycobiology, 2017, 57, 800-806). It binds to Siglec-10 on innate immune cells. Recently it has been shown that CD24 via Siglec-10 acts as an innate immune checkpoint (Barkal et al., Nature. 2019, 572, 392-396).

In some embodiments, the cell outlined herein comprises a nucleotide sequence encoding a CD24 polypeptide has at least 95% sequence identity (e.g., 95%, 96%, 97%, 98%, 99%, or more) to an amino acid sequence set forth in NCBI Ref. Nos. NP_001278666.1, NP_001278667.1, NP_001278668.1, and NP_037362.1. In some embodiments, the cell outlined herein comprises a nucleotide sequence encoding a CD24 polypeptide having an amino acid sequence set forth in NCBI Ref. Nos. NP_001278666.1, NP_001278667.1, NP_001278668.1, and NP_037362.1.

In some embodiments, the cell comprises a nucleotide sequence having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_00129737.1, NM_00129738.1, NM_001291739.1, and NM_013230.3. In some embodiments, the cell comprises a nucleotide sequence as set forth in NCBI Ref. Nos. NM_00129737.1, NM_00129738,1, NM_001291739.1, and NM_013230.3.

In some embodiments, a suitable gene editing system (e.g., CRISPR/Cas system or any of the gene editing systems described herein) is used to facilitate the insertion of a polynucleotide encoding CD24, into a genomic locus of the hypoimmunogenic cell. In some embodiments, the polynucleotide encoding CD24 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is operably linked to a promoter.

In some embodiments, CD24 protein expression is detected using a Western blot of cells lysates probed with antibodies against the CD24 protein. In some embodiments, reverse transcriptase polymerase chain reactions (RT-PCR) are used to confirm the presence of the exogenous CD24 mRNA.

In some embodiments, a suitable gene editing system (e.g., CRISPR/Cas system or any of the gene editing systems described herein) is used to facilitate the insertion of a polynucleotide encoding C024, into a genomic locus of the hypoimmunogenic cell. In some embodiments, the polynucleotide encoding CD24 is inserted into a safe harbor or target locus, such as but not limited to, an AAVS1, CCR5, CLYBL, ROSA26. SHS231, F3 (also known as CD142), MICA, MICB, LRP1 (also known as CD91), HMGB1, ABO, RHD, FUT1, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is operably linked to a promoter.

### K. DUX4

In some embodiments, the present disclosure provides a cell (e.g., stem cell. induced pluripotent stem cell, differentiated cell, hematopoietic stem cell, primary T cell or CAR-T cell) or population thereof comprising a genome modified to increase expression of a tolerogenic or immunosuppressive factor such as DUX4. In some embodiments, the present disclosure provides a method for altering a cell's genome to provide increased expression of DUX4, including through an exogenous polynucleotide. In some embodiments, the disclosure provides a cell or population thereof comprising exogenously expressed DUX4 proteins. In some embodiments, increased expression of DUX4 suppresses, reduces or eliminates expression of one or more of the following MHC I molecules—HLA-A, HLA-B, and HLA-C. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

DUX4 is a transcription factor that is active in embryonic tissues and induced pluripotent stem cells, and is silent in normal, healthy somatic tissues (Feng et al., 2015, ELife4; De laco et al., 2017, Nat Genet, 49, 941-945; Hendrickson et al., 2017, Nat Genet, 49, 925-934; Snider et al., 2010, PLoS Genet, e1001181; Whiddon et al., 2017, Nat Genet). DUX4 expression acts to block IFN-gamma mediated induction of major histocompatibility complex (MHC) class I gene expression (e.g., expression of B2M, HLA-A, HLA-B, and HLA-C). DUX4 expression has been implicated in suppressed antigen presentation by MHC class I (Chew et al., Developmental Cell, 2019, 50, 1-14). DUX4 functions as a transcription factor in the cleavage-stage gene expression (transcriptional) program. Its target genes include, but are not limited to, coding genes, noncoding genes, and repetitive elements.

There are at least two isoforms of DUX4, with the longest isoform comprising the DUX4 C-terminal transcription activation domain. The isoforms are produced by alternative splicing. See, e.g., Gong et al., 2012, Dev Cell, 22, 38-51; Snider et al., 2010, PLoS Genet, e1001181. Active isoforms for DUX4 comprise its N-terminal DNA-binding domains and its C-terminal activation domain. See, e.g., Choi et al., 2016, Nucleic Acid Res, 44, 5161-5173.

It has been shown that reducing the number of CpG motifs of DUX4 decreases silencing of a DUX4 transgene (Jagannathan et al., Human Molecular Genetics, 2016, 25(20):4419-4431). The nucleic acid sequence provided in Jagannathan et al., supra represents a codon altered sequence of DUX4 comprising one or more base substitutions to reduce the total number of CpG sites while preserving the DUX4 protein sequence. The nucleic acid sequence is commercially available from Addgene, Catalog No. 99281.

In many embodiments, at least one or more polynucleotides is utilized to facilitate the exogenous expression of DUX4 by a cell, e.g., a stem cell, induced pluripotent stem cell, differentiated cell, hematopoietic stem cell, primary T cell or CAR-T cell,

In some embodiments, a suitable gene editing system (e.g., CRISPR/Cas system or any of the gene editing systems described herein) is used to facilitate the insertion of a polynucleotide encoding DUX4, into a genomic locus of the hypoimmunogenic cell. In some embodiments, the polynucleotide encoding DUX4 is inserted into a safe harbor or target locus, such as but not limited to, an AAVS1. CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding DUX4 is inserted into a B2M gene locus, a CIITA gene locus, a TRAC gene locus, or a TRB gene locus. In some embodiments, the polynucleotide encoding DUX4 is operably linked to a promoter.

In some embodiments, the polynucleotide encoding DUX4 is inserted into at least one allele of the T cell using viral transduction. In some embodiments, the polynucleotide encoding DUX4 is inserted into at least one allele of the T cell using a lentivirus based viral vector. In some embodiments, the lentivirus based viral vector is a pseudotyped, self-inactivating lentiviral vector that carries the polynucleotide encoding DUX4. In some embodiments, the lentivirus based viral vector is a self-inactivating lentiviral vector pseudotyped with a vesicular stomatitis VSV-G envelope, and which carries the polynucleotide encoding DUX4.

In some embodiments, the polynucleotide sequence encoding DUX4 comprises a polynucleotide sequence comprising a codon altered nucleotide sequence of DUX4 comprising one or more base substitutions to reduce the total number of CpG sites while preserving the DUX4 protein sequence. In some embodiments, the polynucleotide sequence encoding DUX4 comprising one or more base substitutions to reduce the total number of CpG sites has at least 85% (e.g., 85%, 86%, 87%, 88%, 89%, 90%, 91%, 92%, 93%, 94%, 95%, 96%, 97%, 98%, 99% or 100%) sequence identity to SEQ ID NO:1 of PCT/US2020/44635, filed Jul. 31, 2020. In some embodiments, the polynucleotide sequence encoding DUX4 is SEQ ID NO:1 of PCT/US2020/44635.

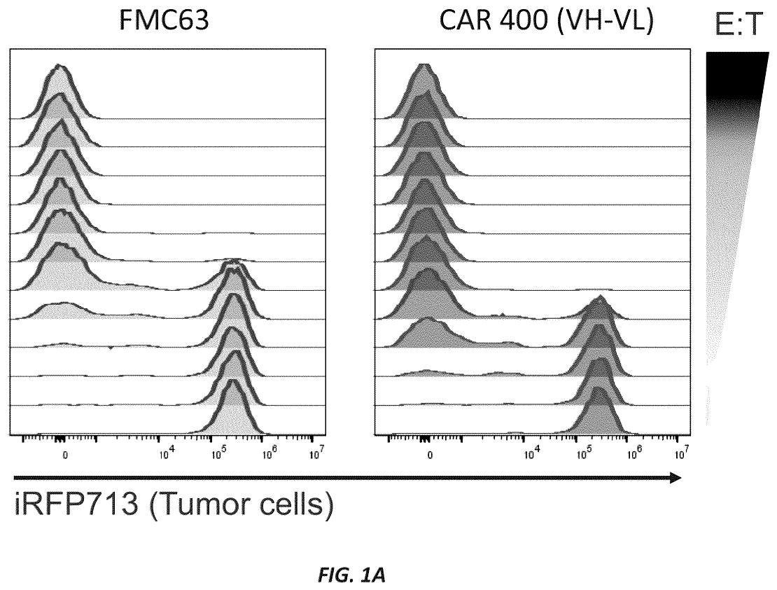

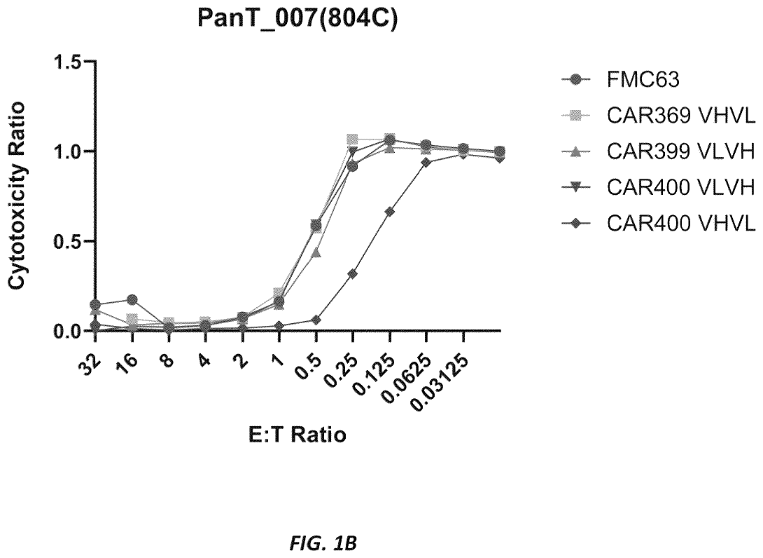

In some embodiments, the polynucleotide sequence encoding DUX4 is a nucleotide sequence encoding a polypeptide sequence having at least 95% (e.g., 95%, 96%, 97%, 98%, 99% or 100%) sequence identity to a sequence selected from a group including SEQ ID NO:2, SEQ ID NO:3, SEQ ID NO:4, SEQ ID NO:5. SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29, as provided in PCT/US2020/44635. In some embodiments, the polynucleotide sequence encoding DUX4 is a nucleotide sequence encoding a polypeptide sequence is selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22. SEQ ID NO:23, SEQ ID NO:24, SEQ ID NO:25, SEQ ID NO:26, SEQ ID NO:27. SEQ ID NO:28, and SEQ ID NO:29. Amino acid sequences set forth as SEQ ID NOs:2-29 are shown in FIG. 1A-1G of PCT/US2020/44635.

In some instances, the DUX4 polypeptide comprises an amino acid sequence having at least 95% sequence identity to the sequence set forth in GenBank Accession No. ACN62209.1 or an amino acid sequence set forth in GenBank Accession No. ACN62209.1. In some instances, the DUX4 polypeptide comprises an amino acid sequence having at least 95% sequence identity to the sequence set forth in NCBI RefSeq No. NP_001280727.1 or an amino acid sequence set forth in NCBI RefSeq No. NP_001280727.1. In some instances, the DUX4 polypeptide comprises an amino acid sequence having at least 95% sequence identity to the sequence set forth in GenBank Accession No. ACP30489.1 or an amino acid sequence set forth in GenBank Accession No. ACP30489.1. In some instances, the DUX4 polypeptide comprises an amino acid sequence having at least 95% sequence identity to the sequence set forth in UniProt No. P0CJ85.1 or an amino acid sequence set forth in UniProt No. P0CJ85.1. In some instances, the DUX4 polypeptide comprises an amino acid sequence having at least 95% sequence identity to the sequence set forth in GenBank Accession No. AUA60622.1 or an amino acid sequence set forth in GenBank Accession No. AUA60622.1. In some instances, the DUX4 polypeptide comprises an amino acid sequence having at least 95% sequence identity to the sequence set forth in GenBank Accession No. ADK24683.1 or an amino acid sequence set forth in GenBank Accession No. ADK24683.1. In some instances, the DUX4 polypeptide comprises an amino acid sequence having at least 95% sequence identity to the sequence set forth in GenBank Accession No. ACN62210.1 or an amino acid sequence set forth in GenBank Accession No. ACN62210.1. In some instances, the DUX4 polypeptide comprises an amino acid sequence having at least 95% sequence identity to the sequence set forth in GenBank Accession No. ADK24706.1 or an amino acid sequence set forth in GenBank Accession No. ADK24706.1. In some instances, the DUX4 polypeptide comprises an amino acid sequence having at least 95% sequence identity to the sequence set forth in GenBank Accession No. ADK24685.1 or an amino acid sequence set forth in GenBank Accession No. ADK24685.1. In some instances, the DUX4 polypeptide comprises an amino acid sequence having at least 95% sequence identity to the sequence set forth in GenBank Accession No. ACP30488.1 or an amino acid sequence set forth in GenBank Accession No. ACP30488.1. In some instances, the DUX4 polypeptide comprises an amino acid sequence having at least 95% sequence identity to the sequence set forth in GenBank Accession No. ADK24687.1 or an amino acid sequence set forth in GenBank Accession No. ADK24687.1. In some instances, the DUX4 polypeptide comprises an amino acid sequence having at least 95% sequence identity to the sequence set forth in GenBank Accession No. ACP30487.1 or an amino acid sequence set forth in GenBank Accession No. ACP30487.1. In some instances, the DUX4 polypeptide comprises an amino acid sequence having at least 95% sequence identity to the sequence set forth in GenBank Accession No. ADK24717.1 or an amino acid sequence set forth in GenBank Accession No. ADK24717.1. In some instances, the DUX4 polypeptide comprises an amino acid sequence having at least 95% sequence identity to the sequence set forth in GenBank Accession No. ADK24690.1 or an amino acid sequence set forth in GenBank Accession No. ADK24690.1. In some instances, the DUX4 polypeptide comprises an amino acid sequence having at least 95% sequence identity to the sequence set forth in GenBank Accession No. ADK24689.1 or an amino acid sequence set forth in GenBank Accession No. ADK24689.1. In some instances, the DUX4 polypeptide comprises an amino acid sequence having at least 95% sequence identity to the sequence set forth in GenBank Accession No. ADK24692.1 or an amino acid sequence set forth in GenBank Accession No. ADK24692.1. In some instances, the DUX4 polypeptide comprises an amino acid sequence having at least 95% sequence identity to the sequence set forth in GenBank Accession No. ADK24693.1 or an amino acid sequence of set forth in GenBank Accession No. ADK24693.1. In some instances, the DUX4 polypeptide comprises an amino acid sequence having at least 95% sequence identity to the sequence set forth in GenBank Accession No. ADK24712.1 or an amino acid sequence set forth in GenBank Accession No. ADK24712.1. In some instances, the DUX4 polypeptide comprises an amino acid sequence having at least 95% sequence identity to the sequence set forth in GenBank Accession No. ADK24691.1 or an amino acid sequence set forth in GenBank Accession No. ADK24691.1. In some instances, the DUX4 polypeptide comprises an amino acid sequence having at least 95% sequence identity to the sequence set forth in UniProt No. P0CJ87.1 or an amino acid sequence of set forth in UniProt No. P0CJ87.1. In some instances, the DUX4 polypeptide comprises an amino acid sequence having at least 95% sequence identity to the sequence set forth in GenBank Accession No. ADK24714.1 or an amino acid sequence set forth in GenBank Accession No. ADK24714.1. In some instances, the DUX4 polypeptide comprises an amino acid sequence having at least 95% sequence identity to the sequence set forth in GenBank Accession No. ADK24684.1 or an amino acid sequence of set forth in GenBank Accession No. ADK24684.1. In some instances, the DUX4 polypeptide comprises an amino acid sequence having at least 95% sequence identity to the sequence set forth in GenBank Accession No. ADK24695.1 or an amino acid sequence set forth in GenBank Accession No. ADK24695.1. In some instances, the DUX4 polypeptide comprises an amino acid sequence having at least 95% sequence identity to the sequence set forth in GenBank Accession No. ADK24699.1 or an amino acid sequence set forth in GenBank Accession No. ADK24699.1. In some instances, the DUX4 polypeptide comprises an amino acid sequence having at least 95% sequence identity to the sequence set forth in NCBI RefSeq No. NP_001768.1 or an amino acid sequence set forth in NCBI RefSeq No. NP_001768. In some instances, the DUX4 polypeptide comprises an amino acid sequence having at least 95% sequence identity to the sequence set forth in NCBI RefSeq No. NP_942088.1 or an amino acid sequence set forth in NCBI RefSeq No. NP_942088.1. In some instances, the DUX4 polypeptide comprises an amino acid sequence having at least 95% sequence identity to SEQ ID NO:28 provided in PCT/US2020/44635 or an amino acid sequence of SEQ ID NO:28 provided in PCT/US2020/44635. In some instances, the DUX4 polypeptide comprises an amino acid sequence having at least 95% sequence identity to SEQ ID NO:29 provided in PCT/US2020/44635 or an amino acid sequence of SEQ ID NO:29 provided in PCT/US2020/44635.

In other embodiments, expression of tolerogenic factors is facilitated using an expression vector. In some embodiments, the expression vector comprises a polynucleotide sequence encoding DUX4 is a codon altered sequence comprising one or more base substitutions to reduce the total number of CpG sites while preserving the DUX4 protein sequence. In some embodiments, the codon altered sequence of DUX4 comprises SEQ ID NO:1 of PCT/US2020/44635. In some embodiments, the codon altered sequence of DUX4 is SEQ ID NO:1 of PCT/US2020/44635. In other embodiments, the expression vector comprises a polynucleotide sequence encoding DUX4 comprising SEQ ID NO:1 of PCT/US2020/44635. In some embodiments, the expression vector comprises a polynucleotide sequence encoding a DUX4 polypeptide sequence having at least 95% sequence identity to a sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29 of PCT/US2020/44635. In some embodiments, the expression vector comprises a polynucleotide sequence encoding a DUX4 polypeptide sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14. SEQ ID NO:15, SEQ ID NO:16. SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29 of PCT/US2020/44635.

An increase of DUX4 expression is assayed using known techniques, such as Western blots. ELISA assays. FACS assays, immunoassays, and the like.

### L. Additional Tolerogenic Factors

In some embodiments, one or more tolerogenic factors is inserted or reinserted into genome-edited cells to create immune-privileged universal donor cells, such as universal donor stem cells, universal donor T cells, or universal donor cells. In some embodiments, the hypoimmunogenic cells disclosed herein have been further modified to express one or more tolerogenic factors. Exemplary tolerogenic factors include, without limitation, one or more of CD47, DUX4, CD24, CD27, CD35, CD46. CD55, CD59, CD200, HLA-C, HLA-E, HLA-E heavy chain, HLA-G, PD-L1, IDO1, CTLA4-Ig, C1-Inhibitor, IL-10, IL-35, IL-39 FasL, CCL21, CCL22, Mfge8, C016, CD52, H2-M3, CD16 Fc receptor, IL15-RF, H2-M3(HLA-G), B2M-HLA-E, A20/TNFAIP3, CR1, HLA-F, and MANF, and Serpinb9. In some embodiments, the tolerogenic factors are selected from the group consisting of CD200, HLA-G, HLA-E, HLA-C, HLA-E heavy chain, PD-1, IDO1, CTLA4-Ig, IL-10, IL-35, FasL, Serpinb9, CCL21, CCL22, and Mfge8. In some embodiments, the tolerogenic factors are selected from the group consisting of DUX4, HLA-C, HLA-E, HLA-F, HLA-G, PD-L1, CTLA-4-Ig, C1-inhibitor, and IL-35. In some embodiments, the tolerogenic factors are selected from the group consisting of HLA-C, HLA-E, HLA-F, HLA-G. PD-L1, CTLA-4-Ig, C1-inhibitor, and IL-35. In some embodiments, the tolerogenic factors are selected from a group including CD47, DUX4, CD24, CD27, C135, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39 Fasl, CCL21, CCL22, Mfge8, CD16, CD52, H2-M3, CD16 Fc receptor, IL15-RF, H2-M3(HLA-G), B2M-HLA-E, A20/TNFAIP3. CR1, HLA-F, and MANF, and Serpinb9.

In some embodiments, the polynucleotide encoding the one or more tolerogenic factors is inserted into at least one allele of the T cell using viral transduction. In some embodiments, the polynucleotide encoding the one or more tolerogenic factors is inserted into at least one allele of the T cell using a lentivirus based viral vector. In some embodiments, the lentivirus based viral vector is a pseudotyped, self-inactivating lentiviral vector that carries the polynucleotide encoding the one or more tolerogenic factors. In some embodiments, the lentivirus based viral vector is a self-inactivating lentiviral vector pseudotyped with a vesicular stomatitis VSV-G envelope, and which carries the polynucleotide encoding the one or more tolerogenic factors.

Useful genomic, polynucleotide and polypeptide information about human CD27 (which is also known as CD27L receptor, Tumor Necrosis Factor Receptor Superfamily Member 7, TNFSF7, T Cell Activation Antigen S152, Tp55, and T14) are provided in, for example, the GeneCard Identifier GC12P008144, HGNC No. 11922, NCBI Gene ID 939, Uniprot No. P26842, and NCBI RefSeq Nos. NM_001242.4 and NP_001233.1.

Useful genomic, polynucleotide and polypeptide information about human CD46 are provided in, for example, the GeneCard Identifier GC01P207752, HGNC No. 6953, NCBI Gene ID 4179, Uniprot No. P15529, and NCBI RefSeq Nos. NM_002389.4, NM_153826.3, NM_172350.2, NM_172351.2, NM_172352.2 NP_758860.1, NM_172353.2, NM_172359.2, NM_172361.2, NP_002380.3, NP_722548.1, NP 758860.1, NP 758861.1, NP_758862.1, NP_758863.1, NP_758869.1, and to NP_758871.1.

Useful genomic, polynucleotide and polypeptide information about human CD55 (also known as complement decay-accelerating factor) are provided in, for example, the GeneCard Identifier GC01P207321, HGNC No. 2665, NCBI Gene ID 1604, Uniprot No. P08174, and NCBI RefSeq Nos. NM_000574.4, NM_001114752.2, NM_001300903.1, NM_001300904.1, NP 000565.1, NP_001108224.1, NP_001287832.1, and NP_001287833.1.

Useful genomic, polynucleotide and polypeptide information about human CD59 are provided in, for example, the GeneCard Identifier GC11M033704, HGNC No. 1689, NCBI Gene ID 966, Uniprot No. P13987, and NCBI RefSeq Nos. NP_000602.1, NM_000611.5, NP_001120695.1, NM_001127223.1, NP_001120697.1, NM_001127225.1, NP_001120698.1, NM_001127226.1, NP_001120699.1, NM_001127227.1, NP 976074.1, NM_203329.2, NP 976075.1, NM 203330.2, NP_976076.1, and NM_203331.2.

Useful genomic, polynucleotide and polypeptide information about human CD200 are provided in. for example, the GeneCard Identifier GC03P112332, HGNC No. 7203, NCBI Gene ID 4345, Uniprot No. P41217, and NCBI RefSeq Nos. NP 001004196.2, NM_001004196.3, NP 001305757.1, NM_001318828.1, NP_005935.4, NM_005944.6, XP_005247539.1, and XM_005247482.2.

Useful genomic, polynucleotide and polypeptide information about human HLA-C are provided in, for example, the GeneCard Identifier GC06M031272, HGNC No. 4933, NCBI Gene ID 3107, Uniprot No. P10321, and NCBI RefSeq Nos. NP_002108.4 and NM_002117.5.

Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5.

Useful genomic, polynucleotide and polypeptide information about human HLA-G are provided in. for example, the GeneCard Identifier GC06P047256, HGNC No. 4964, NCBI Gene ID 3135, Uniprot No. P17693, and NCBI RefSeq Nos. NP_002118.1 and NM_002127.5.

Useful genomic, polynucleotide and polypeptide information about human PD-L1 or CD274 are provided in, for example, the GeneCard Identifier GC09P005450, HGNC No. 17635, NCBI Gene ID 29126, Uniprot No. 09NZQ7, and NCBI RefSeq Nos. NP_001254635.1, NM 001267706.1, NP_054862.1, and NM_014143.3.

Useful genomic, polynucleotide and polypeptide information about human IDO1 are provided in, for example, the GeneCard Identifier GC08P039891, HGNC No. 6059, NCBI Gene ID 3620, Uniprot No. P14902, and NCBI RefSeq Nos. NP_002155.1 and NM_002164.5.

Useful genomic, polynucleotide and polypeptide information about human IL-10 are provided in, for example, the GeneCard Identifier GC01M206767, HGNC No. 5962, NCBI Gene ID 3586, Uniprot No. P22301, and NCBI RefSeq Nos. NP_000563.1 and NM_000572.2.

Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM_000639.2, NP_001289675.1, and NM_001302746.1.

Useful genomic, polynucleotide and polypeptide information about human CCL21 are provided in, for example, the GeneCard Identifier GC09M034709, HGNC No. 10620, NCBI Gene ID 6366. Uniprot No. 000585, and NCBI RefSeq Nos. NP_002980.1 and NM_002989.3.

Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981,2, NM_002990.4, XP_016879020.1, and XM_017023531.1.

Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1. NM_001310320.1, NP_001297250.1, NM_001310321.1, NP_005919.2, and NM_005928.3.

Useful genomic, polynucleotide and polypeptide information about human SerpinB9 are provided in. for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4.

Methods for modulating expression of genes and factors (proteins) include genome editing technologies, RNA or protein expression technologies, and the lIke. For all of these technologies, well known recombinant techniques are used, to generate recombinant nucleic acids as outlined herein.

In some embodiments, the cells (e.g., stem cell, induced pluripotent stem cell, differentiated cell, hematopoietic stem cell, primary T cell or CAR-T cell) possess genetic modifications that inactivate the B2M and CIITA genes and express a plurality of exogenous polypeptides selected from the group including CD47 and DUX4, CD47 and C024, CD47 and CD27, CD47 and CD35, CD47 and CD46, CD47 and CD55. CD47 and CD59, CD47 and CD200, CD47 and HLA-C. CD47 and HLA-E, CD47 and HLA-E heavy chain, CD47 and HLA-G, CD47 and PD-L1, CD47 and IDO1, CD47 and CTLA4-1g, CD47 and C1-Inhibitor, CD47 and IL-10, CD47 and IL-35, CD47 and IL-39, CD47 and FasL, CD47 and CCL21, CD47 and CCL22, CD47 and Mfge8, CD47 and CD16, CD47 and CD52. CD47 and CD16 Fc receptor, CD47 and IL15-RF, CD47 and H2-M3(HLA-G), CD47 and B2M-HLA-E, CD47 and A20/TNFAIP3, CD47 and CR1, CD47 and HLA-F, CD47 and MANF, and CD47 and Serpinb9, and any combination thereof. In some instances, such cells also possess a genetic modification that inactivates the CD142 gene.

In some instances, a gene editing system such as the CRISPR/Cas system is used to facilitate the insertion of tolerogenic factors, such as the tolerogenic factors into a safe harbor or target locus, such as the AAVS1 locus, to actively inhibit immune rejection. In some instances, the tolerogenic factors are inserted into a safe harbor or target locus using an expression vector. In some embodiments, the safe harbor or target locus is an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus.

In some embodiments, expression of a target gene (e.g., DUX4, CD47, or another tolerogenic factor gene) is increased by expression of fusion protein or a protein complex containing (1) a site-specific binding domain specific for the endogenous target gene (e.g., DUX4, CD47, or another tolerogenic factor gene) and (2) a transcriptional activator.

In some embodiments, the regulatory factor is comprised of a site specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs). In some embodiments, the method is achieved by a genome-modifying protein described herein, including for example, a CRISPR-associated transposase, prime editing, or Programmable Addition via Site-specific Targeting Elements (PASTE). In some embodiments, the method is achieved by a genome-modifying protein described herein, including for example, TnpB polypeptides.

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site specific binding domain is derived from a nuclease. For example, the recognition sequences of homing endonucleases and meganucleases such as I-Scel, I-Ceul, PI-Pspl, PI-Sce, I-SceIV, I-Csml, I-Panl, I-Scell, I-Ppol, 1-Scelll, 1-Crel, I-Tevi, I-TevII and I-TevIII. See also U.S. Pat. Nos. 5,420,032; 6,833,252; Belfort et al., (1997) Nucleic Acids Res. 25:3379-3388; Dujon et al., (1989) Gene 82:115-118; Perter et al, (1994) Nucleic Acids Res. 22, 1125-1127; Jasin (1996) Trends Genet. 12:224-228; Gimble et al., (1996) J. Mol. Biol. 263:163-180; Argast et al, (1998) J. Mol. Biol. 280:345-353 and the New England Biolabs catalogue. In some embodiments, the DNA-binding specificity of homing endonucleases and meganucleases are engineered to bind non-natural target sites. See, for example, Chevalier et al. (2002) Molec. Cell 10:895-905; Epinat et al, (2003) Nucleic Acids Res. 31:2952-2962; Ashworth et al, (2006) Nature 441:656-659; Paques et al, (2007) Current Gene Therapy 7:49-66; U.S. Patent Publication No. 2007/0117128.

In some embodiments, Zinc finger, TALE, and CRISPR system binding domains are “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in to length and contains an alpha helix containing two invariant histidine residues coordinated through zinc with two cysteines of a single beta turn, and having two, three, four, five, or six fingers. Generally, sequence-specificity of a ZFP is altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637: Choo et al. (2000) Curr. Opin. Struct. Biol. 10:411-416; U.S. Pat. Nos. 6,453,242; 6,534,261: 6,599,692; 6,503,717; 8,689,558; 7,030,215; 6,794,136; 7,067,317; 7,262,054; 7,070,934; 7,361.635; 7,253,273; and U.S. Patent Publication Nos. 200510064474; 2007/0218528; 2005/0267061, all incorporated herein by reference in their entireties.

Many gene-specific engineered zinc fingers are available commercially. For example. Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-ike protein effector (TALE) protein, See, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. In some embodiments, one or more gRNA are used to target the promoter region of the gene. In some embodiments, one or more regions of the gene are targeted. In some aspects, the target sites are within 600 base pairs on either side of a transcription start site (TSS) of the gene.

It is within the level of a skilled artisan to design or identify a gRNA sequence that is or comprises a sequence targeting a gene, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-cisp.org/E-CRISP/; crispr.mit.edu/). In some embodiments, the gRNA sequence is or comprises a sequence with minimal off-target binding to a non-target gene.

In some embodiments, the regulatory factor further comprises a functional domain, e.g., a transcriptional activator.

In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some embodiments, the transcriptional activator, is or contains all or a portion of an heterologous transactivation domain. For example, in some embodiments, the transcriptional activator is selected from Herpes simplex-derived transactivation domain, Dnmt3a methyttransferase domain, p65, VP16, and VP64.

In some embodiments, the regulatory factor is a zinc finger transcription factor (ZF-TF). In some embodiments, the regulatory factor is VP64-p65-Rta (VPR).

In some embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcd, myb, mos family members etc.); DNA repair enzymes and their associated factors and modifiers; DNA rearrangement enzymes and their associated factors and modifiers; chromatin associated proteins and their modifiers (e.g., kinases, acetylases and deacetylases); and DNA modifying enzymes (e.g., methyltransferases such as members of the DNMT family (e.g., DNMT1, DNMT3A, DNMT3B. DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al, J. Virol. 71, 5952-5962 (1 97)) nuclear hormone receptors (see, e.g., Torchia et al., Curr. Opin. Cell. Biol. 10:373-383 (1998)); the p65 subunit t0 of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, SpI, AP-2, and CTF1 (Seipel et al, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; McKenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, Cl, API. ARF-5, -6, -1, and -8, CPRF1, CPRF4, MYC-RP/GP, and TRAB1, See, for example, Ogawa et al, (2000) Gene 245:21-29; Okanami et al, (1996) Genes Cells 1:87-99; Goff et al. (1991) Genes Dev. 5:298-309; Cho et al, (1999) Plant Mol Biol 40:419-429; Ulmason et al, (1999) Proc. NatI. Acad. Sci. USA 96:5844-5849; Sprenger-Haussels et al, (2000) Plant J. 22:1-8: Gong et al, (1999) Plant Mol. Biol. 41:33-44; and Hobo et al., (1999) Proc. Natl. Acad. Sci. USA 96:15,348-15,353.

Exemplary repression domains that are used to make genetic repressors include, but are not limited to, KRAB A/B. KOX, TGF-beta-inducible early gene (TIEG), v-erbA, SID, MBD2, MBD3, members of the DNMT family (e.g., DNMT1, DNMT3A. DNMT3B, DNMT3L, etc.), Rb, and MeCP2. See, for example, Bird et al, (1999) Cell 99:451-454; Tyler et al, (1999) Cell 99-443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g., type-A, nuclear localized such as MYST family members MOZ, Ybf2/Sas3, MOF, and Tip60, GNAT family members Gcn5 or pCAF, the p300 family members CBP, p300 to or Rttl09 (Bemdsen and Denu (2008) Curr Opin Struct Biol 18(6):682-689). In other instances the domain is a histone deacetylase (HD AC) such as the class I (HDAC-4, 2, 3, and 8), class II (HDAC IIA (HDAC-4, 5, 7 and 9), HD AC 118 (HDAC 6 and 10)), class IV (HDAC-I 1), class III (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2. ATR, ATM, DNA-PK, Bubl, VprBP, IKK-a, PKCpi, Dik/Zip, JAK2, PKCS, WSTF and CK2. In some embodiments, a methylation domain is used and is chosen from groups such as Ezh2, PRMT1/6, PRMT5/7, PRMT 2/6, CARM1, set7/9, MLL, ALL-1, Suv 39h, G9a, SETDB1, Ezh2, Set2, DotI, PRMT 1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

In some embodiments, the present disclosure provides a cell (e.g., a primary T cell and a hypoimmunogenic stem cell and derivative thereof) or population thereof comprising a genome in which the cell genome has been modified to express CD47. In some embodiments, the present disclosure provides a method for altering a cell genome to express CD47. In some embodiments, at least one ribonucleic acid or at least one pair of ribonucleic acids is utilized to facilitate the insertion of CD47 into a cell line. In some embodiments, the at least one ribonucleic acid or the at least one pair of ribonucleic acids is selected from the group consisting of SEQ ID NOs:200784-231885 of Table 29 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-C. In some embodiments, the present disclosure provides a method for altering a cell genome to express HLA-C. In some embodiments, at least one ribonucleic acid or at least one pair of ribonucleic acids is utilized to facilitate the insertion of HLA-C into a cell line. In some embodiments, the at least one ribonucleic acid or the at least one pair of ribonucleic acids is selected from the group consisting of SEQ ID NOs:3278-5183 of Table 10 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-E. In some embodiments, the present disclosure provides a method for altering a cell genome to express HLA-E. In some embodiments, at least one ribonucleic acid or at least one pair of ribonucleic acids is utilized to facilitate the insertion of HLA-E into a cell line. In some embodiments, the at least one ribonucleic acid or the at least one pair of ribonucleic acids is selected from the group consisting of SEQ ID NOs:189859-193183 of Table 19 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-F. In some embodiments, the present disclosure provides a method for altering a cell genome to express HLA-F. In some embodiments, at least one ribonucleic acid or at least one pair of ribonucleic acids is utilized to facilitate the insertion of HLA-F into a cell line. In some embodiments, the at least one ribonucleic acid or the at least one pair of ribonucleic acids is selected from the group consisting of SEQ ID NOs: 688808-399754 of Table 45 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-G. In some embodiments, the present disclosure provides a method for altering a cell genome to express HLA-G. In some embodiments, at least one ribonucleic acid or at least one pair of ribonucleic acids is utilized to facilitate the insertion of HLA-G into a stem cell line. In some embodiments, the at least one ribonucleic acid or the at least one pair of ribonucleic acids is selected from the group consisting of SEQ ID NOs:188372-189858 of Table 18 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express PD-1. In some embodiments, the present disclosure provides a method for altering a cell genome to express PD-L1. In some embodiments, at least one ribonucleic acid or at least one pair of ribonucleic acids is utilized to facilitate the insertion of PD-L1 into a stem cell line. In some embodiments, the at least one ribonucleic acid or the at least one pair of ribonucleic acids is selected from the group consisting of SEQ ID NOs:193184-200783 of Table 21 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express CTLA4-Ig. In some embodiments, the present disclosure provides a method for altering a cell genome to express CTLA4-Ig. In some embodiments, at least one ribonucleic acid or at least one pair of ribonucleic acids is utilized to facilitate the insertion of CTLA4-Ig into a stem cell line. In some embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express Cl-inhibitor. In some embodiments, the present disclosure provides a method for altering a cell genome to express Cl-inhibitor. In some embodiments, at least one ribonucleic acid or at least one pair of ribonucleic acids is utilized to facilitate the insertion of Cl-inhibitor into a stem cell line. In some embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express IL-35. In some embodiments, the present disclosure provides a method for altering a cell genome to express IL-35. In some embodiments, at least one ribonucleic acid or at least one pair of ribonucleic acids is utilized to facilitate the insertion of IL-35 into a stem cell line. In some embodiments, the at least one ribonucleic acid or the at least one pair of ribonucleic acids is selected from any one disclosed in WO2016183041, including the sequence listing.

In some embodiments, the tolerogenic factors are expressed in a cell using an expression vector. In some embodiments, the tolerogenic factors are introduced to the cell using a viral expression vector that mediates integration of the tolerogenic factor sequence into the genome of the cell. For example, the expression vector for expressing CD47 in a cell comprises a polynucleotide sequence encoding CD47. In some embodiments, the expression vector is an inducible expression vector. In some embodiments, the expression vector is a viral vector, such as but not limited to, a lentiviral vector. In some embodiments, the tolerogenic factors are introduced into the cells using fusogen-mediated delivery or a transposase system selected from the group consisting of conditional or inducible transposases, conditional or inducible PiggyBac transposons, conditional or inducible Sleeping Beauty (SB11) transposons, conditional or inducible Mos1 transposons, and conditional or inducible Tol2 transposons.

In some embodiments, the present disclosure provides a cell (e.g., a primary T cell and a hypoimmunogenic stem cell and derivative thereof) or population thereof comprising a genome in which the cell genome has been modified to express any one of the polypeptides selected from the group consisting of HLA-A, HLA-B, HLA-C. RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40. CD27, HVEM, SLAM, CD226, ICOS, LAG3, TIGIT, TIM3, CD180, BTLA, CD244, LFA-1, ST2, HLA-F, CD30, B7-H3, VISTA, TLT, PD-L2, CD58, CD2, HEUOS, and IDO1. In some embodiments, the present disclosure provides a method for altering a cell genome to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA. TLT, PD-L2, CD58, CD2, HELIOS. and IDO1. In some embodiments, at least one ribonucleic acid or at least one pair of ribonucleic acids is utilized to facilitate the insertion of the selected polypeptide into a stem cell line. In some embodiments, the at least one ribonucleic acid or the at least one pair of ribonucleic acids is selected from any one disclosed in Appendices 1-47 and the sequence listing of WO2016183041, the disclosure is incorporated herein by references.

In some embodiments, a suitable gene editing system (e.g.. CRISPR/Cas system or any of the gene editing systems described herein) is used to facilitate the insertion of a polynucleotide encoding a tolerogenic factor, into a genomic locus of the hypoimmunogenic cell. In some embodiments, the polynucleotide encoding the tolerogenic factor is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding the tolerogenic factor is inserted into a 82M gene locus, a CIITA gene locus, a TRAC gene locus, or a TRB gene locus. In some embodiments, the polynucleotide encoding the tolerogenic factor is operably linked to a promoter.

In some embodiments, the cells are engineered to expresses an increased amount of one or more of CD47, DUX4, CD24, CD27, CD35, CD46. CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22. Mfge8, CD16, CD52, H2-M3, CD16 Fc receptor, IL15-RF, H2-M3(HLA-G), B2M-HLA-E, A20/TNFAIP3, CR1, HLA-F, MANF, and/or Serpinb9 relative to a cell of the same cell type that does not comprise the modifications.

### M. Characteristics of Hypoimmune Cells

In some embodiments, the population of hypoimmunogenic stem cells retains pluripotency as compared to a control stem cell (e.g., a wild-type stem cell or immunogenic stem cell). In some embodiments, the population of hypoimmunogenic stem cells retains differentiation potential as compared to a control stem cell (e.g., a wild-type stem cell or immunogenic stem cell).

In some embodiments, the administered population of hypoimmunogenic cells such as hypoimmunogenic CAR-T cells elicits a decreased or lower level of immune activation in the subject or patient. In some instances, the level of immune activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation produced by the administration of immunogenic cells. In some embodiments, the administered population of hypoimmunogenic cells fails to elicit immune activation in the subject or patient.

In some embodiments, the administered population of hypoimmunogenic cells such as hypoimmunogenic CAR-T cells elicits a decreased or lower level of T cell response in the subject or patient. In some instances, the level of T cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of T cell response produced by the administration of immunogenic cells. In some embodiments, the administered population of hypoimmunogenic cells fails to elicit a T cell response to the cells in the subject or patient.

In some embodiments, the administered population of hypoimmunogenic cells such as hypoimmunogenic CAR-T cells elicits a decreased or lower level of NK cell response in the subject or patient. In some instances, the level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cel response produced by the administration of immunogenic cells. In some embodiments, the administered population of hypoimmunogenic cells fails to elicit an NK cell response to the cells in the subject or patient.

In some embodiments, the administered population of hypoimmunogenic cells such as hypoimmunogenic CAR-T cells elicits a decreased or lower level of macrophage engulfment in the subject or patient. In some instances, the level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of macrophage engulfment produced by the administration of immunogenic cells. In some embodiments, the administered population of hypoimmunogenic cells fails to elicit macrophage engulfment of the cells in the subject or patient.

In some embodiments, the administered population of hypoimmunogenic cells such as hypoimmunogenic CAR-T cells elicits a decreased or lower level of systemic TH1 activation in the subject or patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells. In some embodiments, the administered population of hypoimmunogenic cells fails to elicit systemic TH1 activation in the subject or patient.

In some embodiments, the administered population of hypoimmunogenic cells such as hypoimmunogenic CAR-T cells elicits a decreased or lower level of NK cell killing to in the subject or patient. In some instances, the level of NK cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell killing produced by the administration of immunogenic cells. In some embodiments, the administered population of hypoimmunogenic cells fails to elicit NK cell killing in the subject or patient.

In some embodiments, the administered population of hypoimmunogenic cells such as hypoimmunogenic CAR-T cells elicits a decreased or lower level of immune activation of peripheral blood mononuclear cells (PBMCs) in the subject or patient. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells. In some embodiments, the administered population of hypoimmunogenic cells fails to elicit immune activation of PBMCs in the subject or patient.

In some embodiments, the administered population of hypoimmunogenic cells such as hypoimmunogenic CAR-T cells elicits a decreased or lower level of donor-specific IgG antibodies in the subject or patient. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells.

In some embodiments, the administered population of hypoimmunogenic cells fails to elicit donor-specific IgG antibodies in the subject or patient.

In some embodiments, the administered population of hypoimmunogenic cells such as hypoimmunogenic CAR-T cells elicits a decreased or lower level of donor-specific IgM antibodies in the subject or patient. In some instances, the level of donor-specific IgM antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgM antibodies produced by the administration of immunogenic cells. In some embodiments, the administered population of hypoimmunogenic cells fails to elicit donor-specific IgM antibodies in the subject or patient.

In some embodiments, the administered population of hypoimmunogenic cells such as hypoimmunogenic CAR-T cells elicits a decreased or lower level of IgM and IgG antibody production in the subject or patient. In some instances, the level of IgM and is IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells. In some embodiments, the administered population of hypoimmunogenic cells fails to elicit IgM and IgG antibody production in the subject or patient.

In some embodiments, the administered population of hypoimmunogenic cells such as hypoimmunogenic CAR-T cells elicits a decreased or lower level of cytotoxic T cell killing in the subject or patient. In some instances, the level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells. In some embodiments, the administered population of hypoimmunogenic cells fails to elicit cytotoxic T cell killing in the subject or patient.

In some embodiments, the administered population of hypoimmunogenic cells such as hypoimmunogenic CAR-T cells elicits a decreased or lower level of complement-dependent cytotoxicity (CDC) in the subject or patient. In some instances, the level of CDC elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of CDC produced by the administration of immunogenic cells. In some embodiments, the administered population of hypoimmunogenic cells fails to elicit CDC in the subject or patient.

N. Therapeutic Cells from Primary T Cells

Provided herein are hypoimmunogenic cells including, but not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more is healthy humans). In some embodiments, the pool of primary T cells is from 1-100, 1-50, 1-20, 1-10, 1 or more, 2 or more, 3 or more, 4 or more, 5 or more, 10 or more, or more, 30 or more, 40 or more, 50 or more, or 100 or more subjects. In some embodiments, the donor subject is different from the patient (e.g., the recipient that is administered the therapeutic cells). In some embodiments, the pool of T cells do not include cells from the patient. In some embodiments, one or more of the donor subjects from which the pool of T cells is obtained are different from the patient.

In some embodiments, the hypoimmunogenic cells do not activate an innate and/or an adaptive immune response in the patient (e.g., recipient upon administration). Provided are methods of treating a disorder by administering a population of hypoimmunogenic cells to a subject (e.g., recipient) or patient in need thereof. In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the present disclosure is directed to hypoimmunogenic primary T cells that overexpress CD47 and CARs as disclosed herein, and have reduced expression or lack expression of MHC class I and/or MHC class II human leukocyte antigens and have reduced expression or lack expression of TCR complex molecules. The cells outlined herein overexpress CD47 and CARs and evade immune recognition. In some embodiments, the primary T cells display reduced levels or activity of MHC class I antigens, MHC class II antigens, and/or TCR complex molecules. In some embodiments, primary T cells overexpress CD47 and CARs and harbor a genomic modification in the B2M gene. In some embodiments, T cells overexpress CD47 and CARs and harbor a genomic modification in the CIITA gene. In some embodiments, primary T cells overexpress CD47 and CARs and harbor a genomic modification in the TRAC gene. In some embodiments, primary T cells overexpress CD47 and CARs and harbor a genomic modification in the TRB gene. In some embodiments. T cells overexpress CD47 and CARs and harbor genomic modifications in one or more of the following genes: the 82M, CIITA, TRAC and TRB genes.

Exemplary T cells of the present disclosure are selected from the group consisting of cytotoxic T cells, helper T cells, memory T cells, central memory T cells, effector memory T cells, effector memory RA T cells, regulatory T cells, tissue infiltrating lymphocytes, and combinations thereof. In some embodiments, the T cells express CCR7, CD27, CD28, and CD45RA. In some embodiments, the central T cells express CCR7, CD27, CD28, and CD45RO. In other embodiments, the effector memory T cells express PD-1, CD27, CD28, and CD45RO. In other embodiments, the effector memory RA T cells express PD-1, CD57, and CD45RA.

In some embodiments, the T cell is a modified (e.g., an engineered) T cell. In some embodiments, the modified T cell comprise a modification causing the cell to express at least one chimeric antigen receptor as disclosed herein. Useful modifications to primary T cells are described in detail in US2016/0348073 and WO2020/018620, the disclosures of which are incorporated herein in their entireties.

In some embodiments, the hypoimmunogenic cells described herein comprise T cells that are engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells include reduced expression of an endogenous T cell receptor. In some embodiments, the T cells described herein such as the engineered or modified T cells include reduced expression of cytotoxic T-lymphocyte-associated protein 4 (CTLA-4). In other embodiments, the T cells described herein such as the engineered or modified T cells include reduced expression of programmed cell death (PD-1). In some embodiments, the T cells described herein such as the engineered or modified T cells include reduced expression of CTLA-4 and PD-1. Methods of reducing or eliminating expression of CTLA-4, PD-1 and both CTLA-4 and PD-1 are any recognized by those skilled in the art, such as but not limited to, genetic modification technologies that utilize rare-cutting endonucleases and RNA silencing or RNA Interference technologies. Non-limiting examples of a rare-cutting endonuclease include any Cas protein, TALEN, zinc finger nuclease, meganuclease, and homing endonuclease. In some embodiments, an exogenous nucleic acid encoding a polypeptide as disclosed herein (e.g., a chimeric antigen receptor, CD47, or another tolerogenic factor disclosed herein) is inserted at a CTLA-4 and/or PD-1 gene locus. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, the T cells described herein such as the engineered or modified T cells include enhanced expression of PD-1.

In some embodiments, the hypoimmunogenic T cell includes a polynucleotide encoding a CAR as herein disclosed, wherein the polynucleotide is inserted in a genomic locus. In some embodiments, the polynucleotide encoding the CAR is randomly integrated into the genome of the cell. In some embodiments, the polynucleotide encoding the CAR is randomly integrated into the genome of the cell via viral vector transduction. In some embodiments, the polynucleotide encoding the CAR is randomly integrated into the genome of the cell via lentiviral vector transduction. In some embodiments, the polynucleotide is inserted into a safe harbor or target locus, such as but not limited to, an AAVS1, CCR5, CLYBL. ROSA26, SHS231, F3 (also known as CD142), MICA, MICB, LRP1 (also known as CD91), HMGB1, ABO, RHD, FUT1, or KDM5D gene locus. In some embodiments, the polynucleotide is inserted in a B2M, CIITA, TRAC, TRB, PD-1 or CTLA-4 gene.

In some embodiments, the hypoimmunogenic T cell includes a polynucleotide encoding a CAR that is expressed in a cell using an expression vector. In some embodiments, the CAR is introduced to the cell using a viral expression vector that mediates integration of the CAR sequence into the genome of the cell. For example, the expression vector for expressing the CAR in a cell comprises a polynucleotide sequence encoding the CAR. In some embodiments, the expression vector is an inducible expression vector. In some embodiments, the expression vector is a viral vector, such as but not limited to, a lentiviral vector.

Hypoimmunogenic T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia. multiple myeloma, gastric cancer, gastric adenocarcinoma, pancreatic adenocarcinoma, glioblastoma, neuroblastoma, lung squamous cell carcinoma. hepatocellular carcinoma, and bladder cancer.

O. Therapeutic Cells Differentiated from Hypoimmune Pluripotent Stem Cells

Provided herein are hypoimmunogenic cells including, cells derived from pluripotent stem cells, that evade immune recognition. In some embodiments, the cells do not activate an innate and/or an adaptive immune response in the patient or subject (e.g., recipient upon administration). Provided are methods of treating a disorder comprising repeat dosing of a population of hypoimmunogenic cells to a recipient subject in need thereof.

In some embodiments, the pluripotent stem cell and any cell differentiated from such a pluripotent stem cell is modified to exhibit reduced expression of MHC class I human leukocyte antigens. In other embodiments, the pluripotent stem cell and any cell differentiated from such a pluripotent stem cell is modified to exhibit reduced expression of MHC class II human leukocyte antigens. In some embodiments, the pluripotent stem cell and any cell differentiated from such a pluripotent stem cell is modified to exhibit reduced expression of TCR complexes. In some embodiments, the pluripotent stem cell and any cell differentiated from such a pluripotent stem cell is modified to exhibit reduced expression of MHC class I and II human leukocyte antigens. In some embodiments, the pluripotent stem cell and any cell differentiated from such a pluripotent stem cell is modified to exhibit reduced expression of MHC class I and II human leukocyte antigens and TCR complexes.

In some embodiments, the pluripotent stem cell and any cell differentiated from such a pluripotent stem cell is modified to exhibit reduced expression of MHC class I and/or II human leukocyte antigens and exhibit increased CD47 expression. In some instances, the cell overexpresses CD47 by harboring one or more CD47 transgenes. In some embodiments, the pluripotent stem cell and any cell differentiated from such a pluripotent stem cell is modified to exhibit reduced expression of MHC class I and II human leukocyte antigens and exhibit increased CD47 expression. In some embodiments, the pluripotent stem cell and any cell differentiated from such a pluripotent stem cell is modified to exhibit reduced expression of MHC class I and II human leukocyte antigens and TCR complexes and exhibit increased CD47 expression.

In some embodiments, the pluripotent stem cell and any cell differentiated from such a pluripotent stem cell is modified to exhibit reduced expression of MHC class I and/or II human leukocyte antigens, to exhibit increased CD47 expression, and to exogenously express a chimeric antigen receptor as disclosed herein. In some instances, the cell overexpresses CD47 polypeptides by harboring one or more CD47 transgenes. In some instances, the cell overexpresses CAR polypeptides by harboring one or more CAR transgenes. In some embodiments, the pluripotent stem cell and any cell differentiated from such a pluripotent stem cell is modified to exhibit reduced expression of MHC class I and II human leukocyte antigens, exhibit increased CD47 expression, and to exogenously express a chimeric antigen receptor. In some embodiments, the pluripotent stem cell and any cell differentiated from such a pluripotent stem cell is modified to exhibit reduced expression of MHC class I and II human leukocyte antigens and TCR complexes, to exhibit increased CD47 expression, and to exogenously express a chimeric antigen receptor.

Such pluripotent stem cells are hypoimmunogenic stem cells. Such differentiated cells are hypoimmunogenic cells.

In some embodiments, any of the pluripotent stem cells described herein are differentiated into any cells of an organism and tissue. In some embodiments, the cells exhibit reduced expression of MHC class I and/or II human leukocyte antigens and reduced expression of TCR complexes. In some instances, expression of MHC class I and/or II human leukocyte antigens is reduced compared to unmodified or wild-type cell of the same cell type. In some instances, expression of TCR complexes is reduced compared to unmodified or wild-type cell of the same cell type. In some embodiments, the cells exhibit increased CD47 expression. In some instances, expression of CD47 is increased in cells encompassed by the present disclosure as compared to unmodified or wild-type cells of the same cell type. In some embodiments, the cells exhibit exogenous CAR expression. Methods for reducing levels of MHC class I and/or II human leukocyte antigens and TCR complexes and increasing the expression of CD47 and CARs are described herein.

In some embodiments, the cells used in the methods described herein evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject's immune system. In other words, the cells administered in accordance with the methods described herein are not detectable by immune cells of the immune system. In some embodiments, the cells are cloaked and therefore avoid immune rejection.

Methods of determining whether a pluripotent stem cell and any cell differentiated from such a pluripotent stem cell evades immune recognition include, but are not limited to, IFN-γ Elispot assays, microglia killing assays, cell engraftment animal models, cytokine release assays, ELISAs, killing assays using bioluminescence imaging or chromium release assay or a real-time, quantitative microelectronic biosensor system for cell analysis (xCELLigence® RTCA system, Agilent), mixed-lymphocyte reactions, immunofluorescence analysis, etc.

Therapeutic cells outlined herein are useful to treat a disorder such as, but not limited to, a cancer, a genetic disorder, a chronic infectious disease, an autoimmune disorder, a neurological disorder, and the like.

i. T Lymphocytes Differentiated from Hypoimmunogenic Pluripotent Cells

Provided herein, T lymphocytes (T cells, including primary T cells) are derived from the HIP cells described herein (e.g., hypoimmunogenic iPSCs). Methods for generating T cells, including CAR-T cells, from pluripotent stem cells (e.g., iPSCs) are described, for example, in Iriguchi et al., Nature Communications 12,430 (2021); Themeli et al., Cell Stem Cell. 16(4):357-366 (2015); Themeli et al., Nature Biotechnology 31:928-933 (2013).

T lymphocyte derived hypoimmunogenic cells include, but are not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, or more, 30 or more, 40 or more, 50 or more, or 100 or more subjects. In some embodiments, the donor subject is different from the patient (e.g., the recipient that is administered the therapeutic cells). In some embodiments, the pool of T cells does not include cells from the patient. In some embodiments, one or more of the donor subjects from which the pool of T cells is obtained are different from the patient.

In some embodiments, the hypoimmunogenic cells do not activate an immune response in the patient (e.g., recipient upon administration). Provided are methods of treating a disorder by administering a population of hypoimmunogenic cells to a subject (e.g., recipient) or patient in need thereof. In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the HIP-derived T cell includes a chimeric antigen receptor (CAR) as described herein. In some embodiments, any suitable CAR described herein is included in the hyHIP-derived T cell. In some embodiments, the hypoimmunogenic induced pluripotent stem cell-derived T cell includes a polynucleotide encoding a CAR, wherein the polynucleotide is inserted in a genomic locus. In some embodiments, the polynucleotide is inserted into a safe harbor or target locus. In some embodiments, the polynucleotide is inserted in a B2M, CIITA, TRAC, TRB, PD-1 or CTLA-4 gene. In some embodiments, any suitable method is used to insert the CAR into the genomic locus of the hypoimmunogenic cell including the gene editing methods described herein (e.g., a CRISPR/Cas system).

HIP-derived T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

ii. NK Cells Derived from Hypoimmunogenic Pluripotent Cells

Provided herein, natural killer (NK) cells are derived from the HIP cells described herein (e.g., hypoimmunogenic iPSCs).

NK cells (also defined as ‘large granular lymphocytes’) represent a cell lineage differentiated from the common lymphoid progenitor (which also gives rise to B lymphocytes and T lymphocytes). Unlike T-cells, NK cells do not naturally comprise CD3 at the plasma membrane. Importantly, NK cells do not express a TCR and typically also lack other antigen-specific cell surface receptors (as well as TCRs and CD3, they also do not express immunoglobulin B-cell receptors, and instead typically express CD16 and CD56). NK cell cytotoxic activity does not require sensitization but is enhanced by activation with a variety of cytokines including IL-2. NK cells are generally thought to lack appropriate or complete signaling pathways necessary for antigen-receptor-mediated signaling, and thus are not thought to be capable of antigen receptor-dependent signaling, activation and expansion. NK cells are cytotoxic, and balance activating and inhibitory receptor signaling to modulate their cytotoxic activity. For instance, NK cells expressing CD16 may bind to the Fc domain of antibodies bound to an infected cell, resulting in NK cell activation. By contrast, activity is reduced against cells expressing high levels of MHC class I proteins. On contact with a target cell NK cells release proteins such as perforin, and enzymes such as proteases (granzymes). Perforin can form pores in the cell membrane of a target cell, inducing apoptosis or cell lysis.

There are a number of techniques that are used to generate NK cells, including CAR-NK-cells, from pluripotent stem cells (e.g., iPSC); see, for example, Zhu et al., Methods Mol Biol. 2019; 2048:107-119; Knorr et al., Stem Cells Transl Med. 2013 2(4):274-83. doi: 10.5966/sctm.2012-0084; Zeng et al., Stem Cell Reports. 2017 Dec. 12; 9(6):1796-1812; Ni et al., Methods Mol Biol. 2013; 1029:33-41; Bemareggi et al., Exp Hematol. 2019 71:13-23; Shankar et al., Stem Cell Res Ther. 2020; 11(1):234, all of which are incorporated herein by reference in their entirety and specifically for the methodologies and reagents for differentiation. Differentiation is assayed as is known in the art, generally by evaluating the presence of NK cell associated and/or specific markers, including, but not limited to, CD56, KIRs. CD16, NKp44, NKp46, NKG2D, TRAIL, CD122, CD27, CD244, NK1.1, NKG2A/C, NCR1, Ly49, CD49b, CD11b, KLRG1, CD43, CD62L, and/or CD226.

In some embodiments, the hypoimmunogenic pluripotent cells are differentiated into hepatocytes to address loss of the hepatocyte functioning or cirrhosis of the liver.

There are a number of techniques that are used to differentiate HIP cells into hepatocytes; see for example, Pettinato et al., doi: 10.1038/spre32888, Snykers et al., Methods Mol Biol., 2011 698:305-314, Si-Tayeb et al., Hepatology, 2010, 51:297-305 and Asgari et al., Stem Cell Rev., 2013, 9(4):493-504, all of which are incorporated herein by reference in their entirety and specifically for the methodologies and reagents for differentiation. Differentiation is assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release, and glycogen storage.

In some embodiments, the NK cells do not activate an innate and/or an adaptive immune response in the patient (e.g., recipient upon administration). Provided are methods of treating a disorder by administering a population of NK cells to a subject (e.g., recipient) or patient in need thereof. In some embodiments, the NK cells described herein comprise NK cells engineered (e.g., are modified) to express a chimeric antigen receptor including but not limited to a chimeric antigen receptor described herein. In some embodiments, any suitable CAR is included in the NK cells, including the CARs described herein. In some embodiments, the NK cell includes a polynucleotide encoding a CAR, wherein the polynucleotide is inserted in a genomic locus. In some embodiments, the polynucleotide is inserted into a safe harbor or a target locus. In some embodiments, the polynucleotide is inserted in a B2M, CIITA, PD1 or CTLA4 gene. In some embodiments, any suitable method is used to insert the CAR into the genomic locus of the NK cell including the gene editing methods described herein (e.g., a CRISPR/Cas system).

### Methods of Inserting CAR Transgenes to Produce Cells Expressing CARs

In some aspects, the present technology provides methods for generating a population of cells expressing a CAR, such as immune evasive allogeneic T cells, for cell therapy. In some embodiments, the method comprises (a) inserting a first transgene encoding a tolerogenic factor into an endogenous TCR gene locus (e.g., the TRAC and/or TRBC loci including TRBC1 and/or TRBC2) of the T cells, and (b) selecting for T cells that have the transgene inserted by CD3 depletion and/or positive selection for the tolerogenic factor (e.g., selection for expression of the tolerogenic factor). The endogenous TCR gene locus is a genomic locus within any gene encoding a TCR or a component thereof, including, for example, the TRAC and/or TRBC (including TRBC1 and TRBC2) loci. Inserting a tolerogenic factor at the endogenous TCR gene locus may achieve the dual purposes of reducing or eliminating TCR expression and increasing expression of the tolerogenic factor in the T cells (especially allogenic T cells) in one manufacturing step, so that the resulting T cells are made immune evasive and not subject to immune rejection when transplanted into a recipient, thereby increasing both the efficiency of the manufacturing process and the effectiveness of cell-based therapies. In some embodiments, the methods further comprise modifying the expression of MHC class I and/or MHC class II molecules in the T cells. In some embodiments, methods further comprise inserting a second transgene encoding a CAR to a genomic locus of the T cells. A. Insertion of a First Polynucleotide Encoding a Tolerogenic Factor

i. Tolerogenic Factors

In some embodiments, the tolerogenic factor is selected from the group consisting of CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, SERPINB9. CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, and any combinations, truncations, modifications, or fusions of the above.

In some embodiments, the tolerogenic factor is CD47. CD47 is a leukocyte surface antigen and has a role in cell adhesion and modulation of integrins. It is expressed on the surface of a cell (e.g., a T cell) and signals to circulating macrophages not to phagocytize the cell. Overexpression of CD47 thus can reduce the immunogenicity of the cell when grafted and improve immune protection in allogeneic recipients.

In some embodiments, the CD47 is human CD47, and in some of these embodiments, the human CD47 comprises or consists of an amino acid sequence set forth in SEQ ID NO: 167 or SEQ ID NO: 168 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 167 or SEQ ID NO: 168 as set forth in Table 27. In some embodiments, the transgene encoding CD47 comprises a nucleotide sequence corresponding to an mRNA sequence of human CD47. In some embodiments, the transgene encoding CD47 has a nucleotide sequence that is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 169 (coding sequence (CDS) of the nucleotide sequence set forth in NCBI Ref. No. NM 001777.4) or SEQ ID NO: 170 (CDS of the nucleotide sequence set forth in NCBI Ref. No. NM_198793.2).

In some embodiments, the polynucleotide (e.g., transgene) encoding CD47 is codon-optimized for expression in a mammalian cell, for example, a human cell. In some embodiments, the codon-optimized polynucleotide encoding CD47 has a nucleotide sequence that is at least 80% identical (e.g., at least 80%, at least 85%, at least 90%, at least 95%, at least 96%, at least 97%, at least 98%, at least 99%, or 100% identical) to the nucleotide sequence set forth in SEO ID NO: 171.

In some embodiments, a first transgene encoding a first tolerogenic factor at an insertion site at a TCR gene locus has a reverse sequence orientation (5′ to 3′) relative to the sequence of the TCR gene locus. In some embodiments, a first transgene encoding a first tolerogenic factor at an insertion site at a TCR gene locus comprises a promoter that has a reverse sequence orientation (5′ to 3′) relative to the sequence of the TCR gene locus. In some embodiments, the promoter that has a reverse sequence orientation (5′ to 3′) relative to the sequence of the TCR gene locus drives transcription of a first transgene encoding a first tolerogenic factor in a reverse sequence orientation relative to the TCR gene locus. In some embodiments, a first transgene encoding a first tolerogenic factor at an insertion site at a TCR gene locus comprises (in 5′ to 3′ order relative to the TCR gene locus) a poly-A tail sequence, a reverse orientation transgene sequence, and a reverse orientation promoter sequence. In some embodiments, a TCR gene locus comprises a first transgene encoding a first tolerogenic factor and a second transgene encoding a CAR as disclosed herein in a reverse sequence orientation (5′ to 3′) relative to the sequence of the TCR gene locus. In some embodiments, a TCR gene locus comprises a first transgene encoding a first tolerogenic factor in a reverse sequence orientation (5′ to 3′) relative to the sequence of the TCR gene locus and a second transgene encoding a CAR in the forward orientation (i.e., the same orientation) relative to the sequence of the TCR gene locus. In some embodiments, a TCR gene locus comprises a first transgene encoding a first tolerogenic factor and a second transgene encoding a second tolerogenic factor in a reverse sequence orientation (5′ to 3′) relative to the sequence of the TCR gene locus. In some embodiments, a TCR gene locus comprises a first transgene encoding a first tolerogenic factor in a reverse sequence orientation (5′ to 3′) relative to the sequence of the TCR gene locus and a second transgene encoding a second tolerogenic factor in the forward orientation (i.e., the same orientation) relative to the sequence of the TCR gene locus. In some embodiments, a TCR gene locus comprises a first transgene encoding a first tolerogenic factor, a second transgene encoding a second tolerogenic factor, and a third transgene encoding a CAR in a reverse sequence orientation (5′ to 3′) relative to the sequence of the TCR gene locus. In some embodiments, a TCR gene locus comprises a first transgene encoding a first tolerogenic factor and a second transgene encoding a second tolerogenic factor in a reverse sequence orientation (5′ to 3′) relative to the sequence of the TCR gene locus, and a third transgene encoding a CAR in the forward orientation (i.e., the same orientation) relative to the sequence of the TCR gene locus. In some embodiments, a TCR gene locus comprises a first transgene encoding a first tolerogenic factor in a reverse sequence orientation (5′ to 3′) relative to the sequence of the TCR gene locus, a second transgene encoding a second tolerogenic factor in the forward orientation (i.e., the same orientation) relative to the sequence of the TCR gene locus, and a third transgene encoding a CAR in the forward orientation (i.e., the same orientation) relative to the sequence of the TCR gene locus.

ii. Regulatory Elements

In some embodiments, a transgene comprises a gene and one or more regulatory elements. In some embodiments, expression of the tolerogenic factor is operably linked to an endogenous promoter at the TCR gene locus (e.g., TRAC, TRBC1, and/or TRBC2). In some of these embodiments, the first transgene encoding the tolerogenic factor to be inserted need not include an exogenous promoter however, in some embodiments, the transgene may include an exogenous insulator and/or an exogenous enhancer.

Alternatively, in other embodiments, the first transgene encoding a tolerogenic factor may additionally comprise an exogenous promoter to drive expression of the tolerogenic factor in the host cell. In some of these embodiments, the exogenous promoter is one that drives constitutive gene expression in mammalian cells. Those frequently used include, for example, elongation factor 1 alpha (EF1α) promoter, cytomegalovirus (CMV) immediate-early promoter (Greenaway et al., Gene 18: 355-360 (1982)), simian vacuolating virus 40 (SV40) early promoter (Fiers et al., Nature 273:113-120 (1978)), spleen focus-forming virus (SFFV) promoter, phosphoglycerate kinase (PGK) promoter (Adra et al., Gene 60(1):65-74 (1987)), human beta actin promoter, polyubiquitin C gene (UBC) promoter, CAG promoter (Nitoshi et al., Gene 108:193-199 (1991)), MND (MPSV LTR, NCR deleted, and d/587 PBS; Challita et al., J. Virol 69(2):748-755 (1995)) promoter, SSFV promoter, and ICOS promoter. An example of a promoter that is capable of expressing a transgene in a mammalian cell (e.g., a T cell) is the EF1α promoter. The native EF1α promoter drives expression of the alpha subunit of the elongation factor-1 complex, which is responsible for the enzymatic delivery of aminoacyl tRNAs to the ribosome. The EF1α promoter has been extensively used in mammalian expression plasmids and has been shown to be effective in driving CAR expression from transgenes cloned into a lentiviral vector. See, e.g., Milone et al., Mol. Ther. 17(8):1453-1464 (2009). For another example, an MND promoter is a synthetic promoter that contains the U3 region of a modified gammaretrovirus-derived MoMuLV LTR with myeloproliferative sarcoma virus enhancer, and this promoter has been shown to be highly and constitutively active in the hematopoietic system and to resist transcriptional silencing. See, e.g., Halene et al., Blood 94(10):3349-3357 (1999).

In some embodiments, the first transgene encoding a tolerogenic factor may comprise additional regulatory elements operatively linked to the tolerogenic factor sequence and/or promoter, including, for example, insulators, enhancers, polyadenylation (poly(A)) tails, and/or ubiquitous chromatin opening elements. As known to a skilled artisan, these regulatory elements may be needed to affect the expression and processing of coding sequences to which they are operatively linked.

Regulatory elements used for transgene expression modulation may include appropriate transcription initiation, termination, promoter, and enhancer sequences; efficient RNA processing signals, such as splicing and polyadenytation signals; sequences that stabilize cytoplasmic mRNA; sequences that enhance translation efficiency; sequences that enhance protein stability; and possibly sequences that enhance protein secretion.

In some embodiments, the first transgene encoding a tolerogenic factor may additionally comprise an insulator to modulate the expression of the tolerogenic factor in the host cell. Insulators are DNA elements (usually about 50 nucleotides in to length) that can shelter genes from inappropriate regulatory interactions. In some embodiments, insulators insulate genes located in one domain from promiscuous regulation by enhancers or silencers in neighboring domains. Insulators that disrupt communication between an enhancer and its promoter when positioned between the two are called enhancer-blockers, and insulators that are located between a silencer and a promoter and protect the promoter from silencing are caged barriers. In some embodiments, insulators that are barriers prevent the advance of nearby condensed chromatin and protect gene expression from positive and negative chromatin effects. Thus, in the design of a transgene, insulators are usually placed upstream of the promoter. Non-limiting examples of insulators include 5′HS5, DMD/ICR, BEAD-1, apoB (−57 kb), apoB (+43 kb), DM1 site 1, DM1 site 2 (from human); BEAD-1, HS2-6, DMR/ICR, SINE (from mouse); SF1, scs/scs′, gypsy, Fab-7, Fab-8, faswab, eve (from fruit fly); HMR tRNAThr, Chal UAS, UASrpg, STAR (from yeast); Lys 5′A, HS4, or 3′HS (from chicken); sns, URI (from sea urchin); and RO (from frog). Other examples of insulators include Mcp, Neighbor of Homie (Nhomie) insulator and Homing insulator at eve (Homie), and Su(Hw)-dependent insulators. In some embodiments, the first transgene encoding a tolerogenic factor may comprise an insulator having a sequence that is at least 80% identical (e.g., at least 80%, at least 85%, at least 90%, at least 95%, at least 96%, at least 97%, at least 98%, at least 99%, or 100% identical) to any of the described insulators.

In some embodiments, the first transgene encoding a tolerogenic factor comprises one copy of an insulator. In some embodiments, the transgene comprises a multimerized insulator. In some embodiments, a transgene comprises two copies of an insulator. In some embodiments, a transgene comprises three copies of an insulator. In some embodiments, a transgene comprises four copies of an insulator. In some embodiments, a transgene comprises five or more copies of an insulator. Insulator effectiveness is influenced by its structure and by the nature of the enhancer, promoter, and genomic context. In some embodiments, the first transgene encoding a tolerogenic factor may comprise two or more heterologous insulators. In some embodiments, the two or more heterologous insulators interact with each other. In some embodiments, the first transgene encoding a tolerogenic factor comprises an insulator and a regulatory protein that binds to the insulator.

In some embodiments, the first transgene encoding a tolerogenic factor may additionally comprise an enhancer to increase expression of the tolerogenic factor in the host cell. Enhancer sequences are regulatory DNA sequences that, when bound by specific proteins called transcription factors, enhance the transcription of an associated gene. Enhancers are regions of DNA, typically 100 to 1000 bp in size, that contain transcription factor-binding sites that stimulate the initiation and elongation of transcription from promoters. In most housekeeping genes, enhancers are located in close proximity to promoters. Some genes feature complex regulatory regions that can consist of dozens of enhancers located at variable distances from the regulated promoter. During transcriptional activation, enhancers are usually located in dose proximity to gene promoters. Some promoters described herein already have an enhancer incorporated; for example, the CAG promoter is constructed by combining the CMV early enhancer element, the chicken beta actin gene promoter, and the splice acceptor of the rabbit beta globin gene.

Enhancers may consist of combinations of short, degenerate sites, 6-12 bp in length, that are recognized by DNA-binding transcription factors, which determine enhancer activity. The combination of DNA-binding transcription factors on a given enhancer creates a platform that attracts co-activators and co-repressors that determine the enhancer activity in each specific group of cells. The ability of an enhancer to stimulate transcription depends on the combination of transcription factor sites that positively or negatively affect enhancer activity and the relative concentrations of enhancer-binding transcription factors within the nuclei of a given group of cells. Recently, super-enhancers have been identified, representing a special class of regulatory elements, characterized by large sizes, sometimes reaching tens of thousands of bp, with a high degree of transcription factor and co-activator enrichment. Super-enhancers are often located adjacent to genes known to be critical for cell differentiation. A more detailed study of super-enhancers has shown that they often consist of separate domains that can either function together to enhance the overall activity of each domain or play independent roles during the simultaneous activation of a large number of promoters.

During the activation of transcription, enhancers recruit several key complexes. The p300/CBP and MII3/MIW4/COMPASS complexes have acetyltransferase and methyltransferase activities, respectively. The proteins MII3 and MII4 both contain a C-terminal SET (suppressor of variegation, enhancer of zeste, trithorax) domain, which is responsible for the monomethylation of lysine 4 of histone H3 (H3K4me1). The complexes formed by MII3 and MII4 have partially overlapping and insufficiently studied functions in the regulation of enhancer activity. MII3 and MII4 are also known to be involved in the recruitment of the p300/CBP co-activator, which is responsible for the acetylation of histone H3 at lysine 27 (H3K27ac). H3K27ac and H3K4me1 histone marks are distinctive features of active enhancers and are used to identify enhancers in genomes.

In some embodiments, the first transgene encoding a tolerogenic factor may additionally comprise a poly(A) tail. A poly(A) tail is a long chain of adenine nucleotides that is added to an mRNA molecule during RNA processing to increase the stability of the molecule. Immediately after a gene in a eukaryotic cell is transcribed, the new RNA molecule undergoes several modifications known as RNA processing. These modifications alter both ends of the primary RNA transcript to produce a mature mRNA molecule. The processing of the 3′ end adds a poly-A tail to the RNA molecule. First, the 3′ end of the transcript is cleaved to free a 3′ hydroxyl. Then an enzyme called poly-A polymerase adds a chain of adenine nucleotides to the RNA. This process, called polyadenylation, adds a poly-A tail that is between 100 and 250 residues long. The poly-A tail makes the RNA molecule more stable and prevents its degradation. Additionally, the poly-A tail allows the mature messenger RNA molecule to be exported from the nucleus and translated into a protein by ribosomes in the cytoplasm.

In some embodiments, the first transgene encoding a tolerogenic factor may additionally comprise a ubiquitous chromatin opening element (UCOE). The integration of a transgene into a heterochromatic chromatin environment and the methylation of promoter DNA are major mechanisms that are antagonistic to gene expression, resulting in a variegated pattern of gene expression or silencing. Because stable and high level transgene expression are essential for the efficient and rapid production of clonal cell lines in biomanufacturing as well as for the lifelong expression of a transgene at a therapeutic level in gene therapy, genetic regulatory elements that can prevent gene silencing and maintain high levels of expression for long periods of time are crucial.

Genetic regulatory elements that confer a transcriptionally permissive state are broadly dichotomized into those that actively function through dominant chromatin remodeling mechanisms and those that function as border or boundary elements to restrict the spread of heterochromatin marks into regions of euchromatin. The latter include insulators, scaffold/matrix attachment regions (S/MARs), and stabilizing anti-repressor (STAR) elements, whilst the former comprise locus control regions (LCRs) and UCOEs. LCRs and UCOEs are defined by their ability to consistently confer site of integration-independent stable transgene expression that is proportional to transgene copy number, even when integrated into heterochromatin. LCRs are tissue-specific regulatory elements that consist of multiple subcomponents characterized by DNase I hypersensitivity and a high density of transcription factor binding sites. In contrast, UCOEs function ubiquitously and neither consist of multiple DNase I hypersensitive sites that are characteristic of LCRs, nor are they required to flank a transgene at both 5′ and 3′ ends in order to exert their function as in the case of insulators and S/MARs. Thus, structurally and functionally UCOEs represent a distinct class of genetic regulatory element. UCOEs have found widespread usage in protein therapeutic biomanufacturing applications as a means to manage costs and resources as well as to reliably expedite the generation of highly expressing recombinant cell clones. In some embodiments, UCOEs provide stable ubiquitous or tissue-specific expression in somatic tissues as well as in adult, embryonic, and induced pluripotent stem cells and their differentiated progeny.

iii. Site-directed Genomic Insertion

In some embodiments, the first transgene encoding a tolerogenic factor and/or regulatory elements are delivered into a host cell for targeted genomic insertion in the form of a vector or targeted lipid particle. In some embodiments, the delivery vector is any type of vector suitable for introduction of nucleotide sequences into a cell, including, for example, plasmids, adenoviral vectors, adeno-associated viral (AAV) vectors, retroviral vectors, lentiviral vectors, phages, and HDR-based donor vectors. The different components are introduced into a cell together or separately, and are delivered in a single vector or multiple vectors. The vector is introduced into a cell by any known method in the field, including, for example, viral transformation, calcium phosphate transfection, lipid-mediated transfection, DEAE-dextran, electroporation, microinjection, nucleoporation, liposomes, nanoparticles, or other methods. Insertion of the first transgene encoding a tolerogenic factor and/or regulatory elements into an endogenous TCR gene locus is carried out using any of the site-directed insertion methods and/or systems described herein, including, for example, zinc finger nucleases (ZFNs), transcription activator-like effector nucleases (TALENs), meganucleases, transposases, and clustered regularly interspaced short palindromic repeat (CRISPR)/Cas systems. Insertion of the first transgene encoding a tolerogenic factor and/or regulatory elements into an endogenous TCR gene locus is carried out using a genome-modifying protein described herein, including for example, a CRISPR-associated transposase, prime editing, or Programmable Addition via Site-specific Targeting Elements (PASTE). Insertion of the first transgene encoding a tolerogenic factor and/or regulatory elements into an endogenous TCR gene locus is carried out using a genome-modifying protein described herein, including for example, TnpB polypeptides. In embodiments where a homology directed repair (HDR)-based approach as described is used, the transgene is usually flanked by homology arms (i.e., left homology arm (LHA) and right homology arm (RHA)) that are specific to the target site of insertion. The homology arms are specifically designed for the target genomic locus for the fragment to serve as a template for HDR. The length of each homology arm is generally dependent on the size of the insert being introduced, with larger insertions requiring longer homology arms.

B. TCR Depletion, CD3 Depletion, and/or Positive Selection for the Tolerogenic Factor

In some embodiments, the methods described herein for generating a population of T cells, such as immune evasive allogeneic T cells, comprise selecting for cells containing the first transgene encoding a tolerogenic factor integrated into an endogenous TCR gene locus of the T cells, wherein integration of the first transgene into the TCR gene locus reduces or eliminates expression of a functional TCR complex at a surface of the T cells, which in turn prevents CD3 from locating to the cell surface. In some embodiments, the selecting comprises CD3 depletion. In to some embodiments, the selecting comprises positive selection for the tolerogenic factor (e.g., selection for expression of the tolerogenic factor). In some embodiments, CD3 depletion comprises selecting for T cells that have reduced or eliminated expression of endogenous TCR on a cell surface and therefore have reduced or eliminated CD3 associated with a functional TCR complex on the cell surface. In some embodiments, T cells with reduced or eliminated CD3 expression on the cell surface have reduced or eliminated binding to CD3-binding antibodies and/or other CD3-binding proteins. In some embodiments, T cells with reduced or eliminated CD3 expression on the cell surface do not bind to a column and/or a sorting surface with attached CD3-binding antibodies and/or other CD3-binding proteins. In some embodiments, the population of T cells which fails to bind to the CD3-binding antibodies flows through the column and is collected. This population of T cells may also be referred to as enriched for CD3-negative T cells or enriched for T cells having reduced surface expression of CD3. In some embodiments, the selecting comprises TCR depletion. In some embodiments. TCR depletion comprises selecting for T cells that have reduced or eliminated expression of endogenous TCR on a cell surface and therefore have reduced or eliminated TCR complex on the eel surface. In some embodiments, T cells with reduced or eliminated TCR expression on the cell surface have reduced or eliminated binding to TCR-binding antibodies and/or other TCR-binding proteins. In some embodiments, T cells with reduced or eliminated TCR expression on the cell surface do not bind to a column and/or a sorting surface with attached TCR-binding antibodies and/or other TCR-binding proteins. In some embodiments, the population of T cells which fails to bind to the TCR-binding antibodies flows through the column and is collected. This population of T cells may also be referred to as enriched for TCR-negative T cells or enriched for T cells having reduced surface expression of TCR. In some embodiments, positive selection for the tolerogenic factor (e.g., CD47) comprises selecting for T cells that express the tolerogenic factor on the cell surface, for example, at a higher level than endogenous expression levels of the tolerogenic factor. In some embodiments, positive selection for the tolerogenic factor comprises selecting for T cells that express the tolerogenic factor on the cell surface, for example, at a higher level than endogenous expression levels of the tolerogenic factor if the cell expresses any endogenous tolerogenic factor. In these embodiments, antibodies and/or proteins that bind the tolerogenic factor are selected based on a desired affinity and/or avidity for the tolerogenic factor. For example, antibodies and/or proteins having higher affinities and/or avidities for the tolerogenic factor are selected over lower affinities and/or avidities for use with cells which express endogenous levels of the tolerogenic factor. In some embodiments, T cells expressing the tolerogenic factor on the cell surface bind to antibodies and/or proteins that bind to the tolerogenic factor. In some embodiments, T cells expressing the tolerogenic factor on the cell surface bind to a column and/or a sorting surface with attached antibodies and/or other proteins binding the tolerogenic factor.

In some embodiments, the methods described herein for generating a population of T cells, such as immune evasive allogeneic T cells, comprises selecting for cells containing the first transgene encoding a tolerogenic factor integrated into an endogenous TCR gene locus of the T cells, wherein integration of the first transgene into the endogenous TCR gene locus reduces or eliminates expression of a functional TCR complex at a surface of the T cells. In some embodiments, the selecting comprises CD3 depletion, wherein the T cells with reduced or eliminated expression of CD3 on the cell surface are sorted by affinity binding, flow cytometry, and/or immunomagnetic selection using CD3-binding antibodies and/or other CD3-binding proteins. In some embodiments, the selecting comprises TCR depletion, wherein the T cells with reduced or eliminated expression of TCR on the cell surface are sorted by affinity binding, flow cytometry, and/or immunomagnetic selection using TCR-binding antibodies and/or other TCR-binding proteins. In some embodiments, the methods described herein for generating T cells, such as immune evasive allogeneic T cells, comprises selecting for cells containing the first transgene encoding a tolerogenic factor using positive selection for the tolerogenic factor. In some embodiments, the positive selection for the tolerogenic factor comprises selecting for T cells that express the tolerogenic factor on the cell surface by affinity binding, flow cytometry, and/or immunomagnetic selection using antibodies and/or other proteins that bind the tolerogenic factor. In some embodiments, the tolerogenic factor is CD47.

Several methods of sorting living cells based on whether and/or how much they express or do not express a specific protein on their cell surface are known to those of skill in the art. For example, fluorescence activated cell sorting (FACS) of live cells separates a population of cells into sub-populations based on fluorescent labeling using a flow cytometer. Cells stained using fluorophore-conjugated antibodies to an antigen or marker of interest, such as CD3. TCR, or CD47, are separated from one another depending on which fluorophore they have been stained with. For example, a cell expressing one cell marker is detected using an FITC-conjugated antibody that recognizes the marker, and another cell type expressing a different marker could be detected using a PE-conjugated antibody specific for that marker.

Another example of a cell sorting method is magnetic-activated cell sorting (MACS). MACS is a method for separation of various cell populations depending on their surface antigens, such as CD3, TCR, or CD47. The method uses superparamagnetic nanoparticles and columns. The superparamagnetic nanoparticles are of the order of 100 nm. They are used to tag the targeted cells in order to capture them inside the column. The column is placed between permanent magnets so that when the magnetic particle-cell complex passes through it, the tagged cells are captured. The column consists of steel wool which increases the magnetic field gradient to maximize separation efficiency when the column is placed between the permanent magnets. The MACS method allows cells to be separated by using magnetic nanoparticles coated with antibodies against a particular surface antigen, such as CD3, TCR, and/or CD47. This causes the cells expressing this antigen to attach to the magnetic nanoparticles. After incubating the beads and cells, the solution is transferred to a column in a strong magnetic field. In this step, the cells attached to the nanoparticles (expressing the antigen) stay on the column, while other cells (not expressing the antigen) flow through. With this method, the cells are separated positively or negatively with respect to the particular antigen(s). With positive selection, the cells expressing the antigen(s) of interest, which are attached to the magnetic column, are washed out to a separate vessel, after removing the column from the magnetic field. In some embodiments, positive selection methods are used to distinguish cells expressing endogenous tolerogenic factors from cells expressing tolerogenic factors encoded by transgenes. For example, endogenous expression levels of tolerogenic factors are generally lower than expression levels of tolerogenic factors encoded by transgenes. In these instances, a positive selection method could include contacting the cells with beads conjugated to a first antibody against the tolerogenic factor having a first avidity and/or a first affinity which may bind preferentially to cells expressing both exogenous transgene encoded tolerogenic factors as well as endogenous tolerogenic factor molecules. Any cells expressing mostly the endogenous tolerogenic factor would flow through the column. With negative selection, the antibody used is against surface antigen(s) which are known to be present on cells that are not of interest. After administration of the cells/magnetic nanoparticles solution onto the column the cells expressing these antigens bind to the column and the fraction that goes through is collected, as it contains almost no cells with these undesired antigens.

Another example of a cell sorting method is the Streptamer technology, which allows reversible isolation and staining of antigen-specific T cells. In principle, the T cells are separated by establishing a specific interaction between the T cell of interest and a molecule that is conjugated to a marker, which enables the isolation. The reversibility of this interaction and the fact that it is performed at low temperatures is the reason for the successful isolation and characterization of functional T cells. Because T cells remain phenotypically and functionally indistinguishable from untreated cells, this method offers new strategies in clinical and basic T cell research. The Streptamer staining principle combines the classic method of T cell isolation by MHC-multimers with the Strep-tag/Strep-Tactin technology. The Strep-tag is a short peptide sequence that displays moderate binding affinity for the biotin-binding site of a mutated streptavidin molecule, called Strep-Tactin. For the Streptamer technology, the Strep-Tactin molecules are multimerized, thus creating a platform for binding to strep-tagged proteins. Further, the Strep-Tactin backbone has a fluorescent label to allow flow cytometry analysis. Incubation of MHC-Strep-tag fusion proteins with the Strep-Tactin backbone results in the formation of an MHC-multimer, which is capable for antigen-specific staining of T cells.

Other examples of cell separation using methodological standards that ensure high purity are rapid and label-free separation procedures based on surface marker density. Exemplary procedures involve the use of an anti-surface marker antibody-immobilized cell-rolling column, that can separate cells depending on the surface marker density of the cell surfaces. In some embodiments, various conditions for the cell-rolling column are optimized including adjustment of the column tilt angle and medium flow rate.

In some embodiments, the T cells generated by methods according to various embodiments of the present technology have at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the T cells in the population having the first transgene encoding a tolerogenic factor (e.g.. CD47) inserted into an endogenous TCR gene locus. In some embodiments, have at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the generated T cells have reduced expression of CD3 and/or increased expression of a tolerogenic factor (e.g., CD47) encoded by a transgene. In some embodiments, have at least 30%, at least 35%, at least 40%, at least 45%, at least 50%, at least 55%, at least 80%, at least 65%, at least 70%, at least 75%, at least 80%, at least 85%, at least 90%, at least 95%, or at least 100% of the generated T cells have reduced expression of TCR and/or increased expression of a tolerogenic factor (e.g., CD47) encoded by a transgene. In any of these embodiments, the remainder T cells in the population do not possess the described selection characteristic(s).

### C. Insertion of a Second Transgene Encoding a CAR

In some embodiments, the methods described herein for generating a population of cells, such as immune evasive allogeneic T cells, may further comprise inserting a second transgene encoding one or more CARs to a genomic locus of the T cells, in order to generate CAR-T cells for use in cell-based therapies against various target antigens and/or cell surface molecules. This step of inserting a second transgene encoding one or more CARs may occur before, with, or after the step of inserting a first transgene encoding a tolerogenic factor. In some embodiments, the CAR is a CD19 CAR, and in these embodiments, the second transgene comprises a nucleotide sequence encoding a CD19 CAR as disclosed herein.

i. Multiple CARs

In some embodiments, the second transgene comprises two or more nucleotide sequences, each encoding a CAR targeting a specific target antigen as herein disclosed. In these embodiments, the second transgene encodes two or more different CARs specific to different target cell surface molecules or antigens (e.g., a CD19 CAR and a CD22 CAR). The two or more CARs may each comprise an extracellular binding domain specific to a specific target cell surface molecule, and may comprise the same, or one or more different, non-antigen binding domains. For is example, the two or more CARs may comprise different signal peptides, hinge domains, transmembrane domains, costimulatory domains, and/or intracellular signaling domains, in order to minimize the risk of recombination due to sequence similarities. Or, alternatively, the two or more CARs may comprise the same non-antigen binding domains. In the embodiments where the same non-antigen binding domain(s) and/or backbone are used, it is optional to introduce codon divergence at the nucleotide sequence level to minimize the risk of recombination. As one non-limiting example, the second transgene may comprise a nucleotide sequence encoding a CD19 CAR and a nucleotide sequence encoding a CD22 CAR. The CD19 CAR may comprise one transmembrane domain (e.g., CD28 transmembrane domain) while the CD22 CAR comprises a different transmembrane domain (e.g., CD8c transmembrane domain), or vice versa. As another non-limiting example, the CD19 CAR may comprise one costimulatory domain (e.g., 4-1BB costimulatory domain) while the CD22 CAR comprises a different costimulatory domain (e.g., CD28 costimulatory domain), or vice versa. Or, alternatively, the CD22 CAR and the CD19 CARs may comprise the same non-antigen binding domains but have codon divergence introduced at the nucleotide sequence level to minimize the risk of recombination. In any of these embodiments, the two or more nucleotide sequences of the second transgene are connected by one or more cleavage sites as described (e.g., a 2A site and/or a furin site), in the form of polycistronic constructs as described herein.

ii. Regulatory Elements

In some embodiments, the second transgene encoding a CAR may comprise additional regulatory elements operatively linked to the CAR encoding sequence as described, including, for example, promoters, insulators, enhancers, polyadenylation (poly(A)) tails, and/or ubiquitous chromatin opening elements.

### D. Genomic Insertion

In some embodiments, the second transgene encoding a CAR is delivered into a host cell in the form of a vector for insertion into the host genome. In some embodiments, the insertion is random (i.e., insertion into a random genomic locus of the host cell) or targeted (i.e., insertion into a specific genomic locus of the host cell), using any of the random or site-directed insertion methods described herein.

In some embodiments, the first transgene encoding a tolerogenic factor and the second transgene encoding a CAR are introduced into a host for genomic insertion separately. In some embodiments, the first transgene encoding a tolerogenic factor and the second transgene encoding a CAR are introduced into a host for genomic insertion at the same time, via a single vector or multiple vectors. In embodiments where the first and the second transgene are delivered into a host cell together in a single vector, the first and the second transgene are designed as a polycistronic construct as described below.

### E. Polycistronic Constructs

In some embodiments, the first transgene encoding a tolerogenic factor and the second transgene encoding a CAR, and/or the multiple CAR encoding sequences of the second transgene, are in the form of polycistronic constructs. Polycistronic constructs have two or more expression cassettes for co-expression of two or more proteins of interest in a host cell. In some embodiments, the polycistronic construct comprises two expression cassettes, i.e., is bicistronic. In some embodiments, the polycistronic construct comprises three expression cassettes, i.e., is tricistronic. In some embodiments, the polycistronic construct comprises four expression cassettes, i.e., is quadcistronic. In some embodiments, the polycistronic construct comprises more than four expression cassettes. In any of these embodiments, each of the expression cassettes comprises a nucleotide sequence encoding a protein of interest (e.g., a tolerogenic factor, a suicide switch, a regulatory factor, an antibody or antigen binding fragment thereof, or a CAR). In some embodiments, the two or more genes being expressed are under the control of a single promoter and are separated from one another by one or more cleavage sites to achieve co-expression of the proteins of interest from one transcript. In other embodiments, the two or more genes are under the control of separate promoters.

In some embodiments, the two or more expression cassettes of the polycistronic construct are separated by one or more cleavage sites. As the name suggests, a polycistronic construct allows simultaneous expression of two or more separate proteins from one mRNA transcript in a host cell. Cleavage sites are used in the design of a polycistronic construct to achieve such co-expression of multiple genes.

In some embodiments, the one or more cleavage sites comprise one or more self-cleaving sites. In some embodiments, the self-cleaving site comprises a 2A site. 2A peptides are a class of 18-22 amino acid-long peptides first discovered in picornaviruses and can induce ribosomal skipping during translation of a protein, thus producing equal amounts of multiple genes from the same mRNA transcript. 2A peptides function to “cleave” an mRNA transcript by making the ribosome skip the synthesis of a peptide bond at the C-terminus, between the glycine (G) and proline (P) residues, leading to separation between the end of the 2A sequence and the next peptide downstream. There are four 2A peptides commonly employed in molecular biology, T2A, P2A, E2A, and F2A, the sequences of which are summarized in Table 28. A glycine-serine-glycine (GSG) linker is optionally added to the N-terminal of a 2A peptide to increase cleavage efficiency. The use of “( )” around a sequence in the present disclosure means that the enclosed sequence is optional.

In some embodiments, the one or more cleavage sites additionally comprise one or more protease sites. The one or more protease sites can either precede or follow the self-cleavage sites (e.g., 2A sites) in the 5′ to 3′ order. The protease site is cleaved by a protease after translation of the full transcript or after translation of each expression cassette such that the first expression product is released prior to translation of the next expression cassette. In these embodiments, having a protease site in addition to the 2A site, especially preceding the 2A site in the 5′ to 3′ order, may reduce the number of extra amino acid residues attached to the expressed proteins of interest. In some embodiments, the protease site comprises a furin site, also known as a Paired basic Amino acid Cleaving Enzyme (PACE) site. There are at least three furin cleavage sequences, FC1, FC2, and FC3, the amino acid sequences of which are summarized in Table 29. In some embodiments, one or more optional glycine-serine-glycine (GSG) sequences are included for cleavage efficiency.

In some embodiments, the one or more cleavage sites comprise one or more self-cleaving sites, one or more protease sites, and/or any combination thereof. For example, the cleavage site includes a 2A site alone. For another example, the cleavage site includes a FC2 or FC3 site, followed by a 2A site. In these embodiments, the one or more self-cleaving sites are the same or different. In some embodiments, the one or more protease sites are the same or different.

In some embodiments, the polycistronic construct are in the form of a vector. In some embodiments, any type of vector suitable for introduction of nucleotide sequences into a host cell is used, including, for example, plasmids, adenoviral vectors, adenoviral-associated vectors, retroviral vectors, lentiviral vectors, phages, and homology-directed repair (HDR)-based donor vectors.

### Gene Editing Systems for Insertion of Polynucleotide Encoding CAR

In some aspects, the first polynucleotide encoding a tolerogenic factor and/or the second polynucleotide encoding a CAR, or the polycistronic construct as herein disclosed are integrated into the genome of a host cell (e.g., a T cell) using methods and compositions described herein.

### A. Random Insertion

In some embodiments, the first polynucleotide encoding a tolerogenic factor and/or the second polynucleotide encoding a CAR are inserted into a random genomic locus of a host cell. As known to a person skilled in the art, viral vectors, including, for example, retroviral vectors, lentiviral vectors, adenoviral vectors, and adeno-associated viral vectors, are commonly used to deliver genetic material into host cells and randomly insert the foreign or exogenous gene into the host cell genome to facilitate stable expression and replication of the gene.

### B. Site-Directed Insertion (Knock-In)

In some embodiments, the first polynucleotide encoding a tolerogenic factor and/or the second polynucleotide encoding a CAR are inserted into a specific genomic locus of the host cell. A number of gene editing methods are used to insert a polynucleotide (e.g., transgene) into a specific genomic locus of choice. Gene editing is a type of genetic engineering in which a nucleotide sequence is inserted, deleted, modified, or replaced in the genome of a living organism. In some embodiments, the gene editing technologies are systems involving nucleases, integrases, transposases, and/or recombinases. In some embodiments, the gene editing technology mediates single-strand breaks (SSB). In some embodiments, the is gene editing technology mediates double-strand breaks (DSB), including in connection with non-homologous end-joining (NHEJ) or homology-directed repair (HDR). In some embodiments, the gene editing technologies are DNA-based editing or prime-editing. In some embodiments, the gene editing technology is Programmable Addition via Site-specific Targeting Elements (PASTE). In some embodiments, the gene editing technology is TnpB polypeptides. Many gene editing techniques generally utilize the innate mechanism for cells to repair double-strand breaks (DSBs) in DNA.

Eukaryotic cells repair DSBs by two primary repair pathways: non-homologous end-joining (NHEJ) and homology-directed repair (HDR). HDR typically occurs during late S phase or G2 phase, when a sister chromatid is available to serve as a repair template. NHEJ is more common and can occur during any phase of the cell cycle, but it is more error prone. In gene editing, NHEJ is generally used to produce insertion/deletion mutations (indels), which can produce targeted loss of function in a target gene by shifting the open reading frame (ORF) and producing alterations in the coding region or an associated regulatory region. HDR, on the other hand, is a preferred pathway for producing targeted knock-ins, knockouts, or insertions of specific mutations in the presence of a repair template with homologous sequences.

Several methods are known to a skilled artisan to improve HDR efficiency, including, for example, chemical modulation (e.g., treating cells with inhibitors of key enzymes in the NHEJ pathway); timed delivery of the gene editing system at S and G2 phases of the cell cycle; cell cycle arrest at S and G2 phases; and introduction of repair templates with homology sequences. The methods provided herein may utilize HDR-mediated repair, NHEJ-mediated repair, or a combination thereof.

In some embodiments, the methods provided herein for HDR-mediated insertion utilize a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems.

i. ZFNs

ZFNs are fusion proteins comprising an array of site-specific DNA binding domains adapted from zinc linger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3-to 4-bp DNA sequence. Tandem domains can thus potentially bind to an extended nucleotide sequence that is unique within a cell's genome.

Various zinc fingers of known specificity are combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers are engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5 overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

ii. TALENs

TALENs are another example of an artificial nuclease which are used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39:e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease is produced specific to any desired DNA sequence. Similar to ZFNs, TALENs are introduced into a cell to generate DSBs at a desired target site in the genome, and so are used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

iii. Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mot Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA is introduced into the cell along with the meganuclease. Depending on the sequences of the foreign DNA and chromosomal sequence, this process is used to modify the target gene. See Silva et al., Current Gene Therapy (2011) 11:11-27.

iv. Transposases

Transposases are enzymes that bind to the end of a transposon and catalyze its movement to another part of the genome by a cut and paste mechanism or a replicative transposition mechanism. By linking transposases to other systems such as the CRISPR/Cas system, new gene editing tools are developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

v. CRISPR/Cas

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types 11, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10). Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. See, e.g., Jinek et al., Science (2012) 337 (6096):816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; Yan et al., Science (2019) 363:88-91. The most widely used Cas9 is a type II Cas protein and is described herein as illustrative. In some embodiments, these Cas proteins are originated from different source species. For example, in some embodiments, Cas9 is derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those described to this point. For instance, Cpf1 (CRISPR from Prevotella and Franciscella 1; also known as Cas12a) is an RNA-guided nuclease that only requires a crRNA and does not need a tracrRNA to function.

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complexes, including in some embodiments via a single gRNA. For example, in some embodiments, the gRNAs are single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” is any nucleotide. Other Cas nuclease variants with alternative PAMs have also been characterized and successfully used for genome editing, which are summarized in Table 30.

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example, the Cas nuclease may have one or more mutations that alter its PAM specificity.

In some embodiments, CRISPR systems of the present disclosure comprise TnpB polypeptides. In some embodiments, TnpB polypeptides may comprise a Ruv-C-like domain. In some embodiments, the RuvC domain is a split RuvC domain comprising RuvC-I, RuvC-II, and RuvC-III subdomains. In some embodiments, a TnpB may further comprise one or more of a HTH domain, a bridge helix domain and a zinc finger domain. TnpB polypeptides do not comprise an HNH domain. In one exemplary embodiment, a TnpB protein comprises, starting at the N-terminus: a HTH domain, a RuvC-I subdomain, a bridge helix domain, a RuvC-II sub-domain, a zinger finger domain, and a RuvC-III sub-domain. In some embodiments, a RuvC-III sub-domain forms the C-terminus of a TnpB polypeptide. In some embodiments, a TnpB polypeptide is from Epsilonproteobacteria bacterium, Actinoplanes lobatus strain DSM 43150, Actinomadura celluolosilytica strain DSM 45823, Actinomadura namibiensis strain DSM 44197, Alicyclobacillus macrosprangiidus strain DSM 17980. Lipingzhangella halophila strain DSM 102030, or Ktedonobacter recemifer. In some embodiments, a TnpB polypeptide is from Ktedonobecter racemifer, or comprises a conserved RNA region with similarity to the 5′ ITR of K, racemifer TnpB loci. In some embodiments, a TnpB may comprise a Fanzor protein, a TnpB homolog found in eukaryotic genomes. In some embodiments, a CRISPR system comprising a TnpB polypeptide binds a target adjacent motif (TAM) sequence 5′ of a target polynucleotide. In some embodiments, a TAM is a transposon-associated motif. In some embodiments, a TAM sequence comprises TCA. In some embodiments, a TAM sequence comprises TTCAN. In some embodiments, a TAM sequence comprises TTGAT. In some embodiments, a TAM sequence comprises ATAAA.

In some embodiments, the first and/or the second transgene may function as a DNA repair template to be integrated Into the target site through HDR in associated with a gene editing system (e.g., the CRISPR/Cas system) as described. Generally, the transgene to be inserted would comprise at least the expression cassette encoding the protein of interest (e.g., the tolerogenic factor or CAR) and would optionally also include one or more regulatory elements (e.g., promoters, insulators, enhancers). In some of these embodiments, the transgene to be inserted would be flanked by homologous sequence immediately upstream and downstream of the target, i.e., left homology arm (LHA) and right homology arm (RHA), specifically designed for the target genomic locus to serve as template for HDR. The length of each homology arm is generally dependent on the size of the insert being introduced, with larger insertions requiring longer homology arms.

In some embodiments, target-primed reverse transcription (TPRT) or prime editing is used to engineer exogenous genes, such as exogenous transgenes encoding a tolerogenic factor (e.g., CD47) into specific loci. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some embodiments, prime editing is thought of as a “search-and-replace” genome editing technology since the prime editors search and locate the desired target site to be edited, and encode a replacement strand containing a desired edit which is installed in place of the corresponding target site endogenous DNA strand at the same time. For example, in some embodiments, prime editing is adapted for conducting precision CRISPR/Cas-based genome editing in order to bypass double stranded breaks. In some embodiments, a homologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, a prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, a gene editing technology is associated with a prime editor that is a reverse transcriptase, or any DNA polymerase known in the art. Thus, in one aspect, a prime editor may comprise Cas9 (or an equivalent napDNAbp) which is programmed to target a DNA sequence by associating it with a specialized guide RNA (i.e., PEgRNA) containing a spacer sequence that anneals to a complementary to protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the base editing technology is used to introduce single-nucleotide variants (SNVs) into DNA or RNA in living cells. Base editing is a CRISPR-Cas9-based genome editing technology that allows the introduction of point mutations in RNAs or DNAs without generating DSBs. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some embodiments, base editors may also include proteins or domains that alter cellular DNA repair processes to increase the efficiency and/or stability of the resulting single-nucleotide change. Two major classes of base editors have been developed: cytidine base editors (CBEs) (e.g., BE4) that allow C:G to T:A conversions and adenine base editors (ABEs) (e.g., ABE7.10) that allow A:T to G:C conversions. Base editors are composed by a catalytically dead Cas9 (dCas9) or a nickase Cas9 (nCas9) fused to a deaminase and guided by a sgRNA to the locus of interest. The d/nCas9 recognizes a specific PAM sequence and the DNA unwinds thanks to the complementarity between the sgRNA and the DNA sequence usually located upstream of the PAM (also called protospacer). Then, the opposite DNA strand is accessible to the deaminase that converts the bases located in a specific DNA stretch of the protospacer. Compared to HDR-based strategies, base editing is a promising tool to precisely correct genetic mutations as it avoids gene disruption by NHEJ associated with failed HDR-mediated gene correction. Rat deaminase APOBEC1 (rAPOBEC1) fused to deactivated Cas9 (dCas9) has been used to successfully convert cytidines to thymidines upstream of the PAM of the sgRNA. In some embodiments, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, a base editor is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, a base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, a base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, a base editor is an adenine-to-thymine or “ATBE” (or thymine-to-adenine or “TABE”) transversion base editor. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, a gene editing technology is Programmable Addition via Site-specific Targeting Elements (PASTE). In some aspects, PASTE is platform in which genomic insertion is directed via a CRISPR-Cas9 nickase fused to both a reverse transcriptase and serine integrase. As described in loannidi et al. (doi.org/10.1101/2021.11.01.466786), PASTE does not generate double stranded breaks, but allows for integration of sequences as large as ~36 kb. In some embodiments, a serine integrase is any known in the art. In some embodiments, a serine integrase has sufficient orthogonality such that PASTE is used for multiplexed gene integration, simultaneously integrating at least two different genes at least two genomic loci. In some embodiments, PASTE has editing efficiencies comparable to or better than those of homology directed repair or non-homologous end joining based integration, with activity in non-dividing cells and fewer detectable off-target events.

### C. Genomic Loci for Insertion of the First Polynucleotide

In some embodiments, the genomic locus for site-directed insertion of the first polynucleotide (e.g., transgene) encoding a tolerogenic factor is an endogenous TCR gene locus. In some embodiments, the endogenous TCR gene locus is selected from the group consisting of a TRAC locus, a TRBC1 locus, and a TRBC2 locus. The specific site for insertion within a gene locus is located within any suitable region of the gene, including but not limited to a gene coding region (also known as a coding sequence or “CDS”), an exon, an intron, a sequence spanning a portion of an exon and a portion of an adjacent intron, or a regulatory region (e.g., promoter, enhancer). In some embodiments, the insertion occurs in one allele of the specific genomic locus. In some embodiments, the insertion occurs in both alleles of the specific genomic locus. In either of these embodiments, the orientation of the polynucleotide inserted into the target genomic locus is either the same or the reverse of the direction of the endogenous gene in that locus.

i. TRAC

TCRs recognize foreign antigens which have been processed as small peptides and bound to MHC molecules at the surface of antigen presenting cells (APC). Each TCR is a dimer consisting of one alpha and one beta chain (most common) or one delta and one gamma chain. The genes encoding the TCR alpha chain are clustered on chromosome 14. The TCR alpha chain is formed when one of at least 70 variable (V) genes, which encode the N-terminal antigen recognition domain, rearranges to 1 of 61 joining (J) gene segments to create a functional variable region that is transcribed and spliced to a constant region gene segment encoding the C-terminal portion of the molecule. The beta chain, on the other hand, is generated by recombination of the V, D (diversity), and J segment genes.

The TRAC gene encodes the TCR alpha chain constant region. The human TRAC gene resides on chromosome 14 at 22,547,506-22,552,156, forward strand. The TRAC genomic sequence is set forth in Ensembl ID ENSG00000277734.

ii. TRBC1 and TRBC2

The TRBC gene encodes the TCR beta chain constant region. TRBC1 and TRBC2 are analogs of the same gene, and T cells mutually exclusively express either TRBC1 and TRBC2. The human TRBC1 gene resides on chromosome 7 at 142,791,694-142.793,368, forward strand, and its genomic sequence is set forth in Ensembl ID ENSG00000211751. The human TRBC2 gene resides on chromosome 7 at 142,801,041-142,802,748, forward strand, and its genomic sequence is set forth In Ensembl ID ENSGO0000211772.

### D. Genomic Loci for Insertion of the Second Polynucleotide

In some embodiments, the genomic locus for insertion of the second polynucleotide encoding a CAR as disclosed herein is a random locus (by random insertion) or a specific locus (by site-directed insertion). If a specific locus is desired, it is the same as or a different locus from that of the first transgene. In some embodiments, the genomic locus for insertion of the second transgene encoding a CAR is a specific locus selected from the group consisting of a TRAC locus, a TRBC1 locus, a TRBC2 locus, a B2M locus, a CIITA locus, and a safe harbor locus. Non-limiting examples of safe harbor loci include, but are not limited to, an AAVS1 (also known as PPP1R12C), ABO, CCR5, CLYBL, CXCR4, F3 (also known as CD142), FUT1, HMGB1, KDMSD, LRP1 (also known as CD91), MICA, MICB, RHD, ROSA26, and SHS231 gene locus. In some embodiments, the genomic locus for insertion of the second transgene encoding a CAR is a specific locus comprising a TRAC locus, a TRBC1 locus, a TRBC2 locus, a B2M locus, a CIITA locus, an AAVS1 (also known as PPP1R12C) locus, an ABO locus, a CCR5 locus, a CLYBL locus, aCXCR4locus, an F3 (also known as CD142) locus, a FUT1 locus, an HMGB1 locus, a KDM5D locus, an LRP1 (also known as C091) locus, a MICA locus, an MICB locus, an RHD locus, a ROSA26 locus, or an SHS231 locus. The second polynucleotide is inserted within any suitable region of any of the described locus, including but not limited to a gene coding region (also known as a coding sequence or “CDS”), an exon, an intron, a sequence spanning a portion of an exon and a portion of an adjacent intron, or a regulatory region (e.g., promoter, enhancer). In some embodiments, the insertion occurs in one allele of the genomic locus. In some embodiments, the insertion occurs in both alleles of the genomic locus. In either of these embodiments, the orientation of the polynucleotide inserted into the genomic locus is either the same or the reverse of the direction of the original gene in that locus. In some embodiments, the second polynucleotide Is inserted with the first polynucleotide such as the first polynucleotide and the second polynucleotide are carried by a polycistronic vector.

E. Guide RNAs (gRNAs) for Site-Directed Insertion

In some embodiments, provided are gRNAs for use in site-directed insertion of a polynucleotide in according to various embodiments provided herein, especially in association with the CRISPR/Cas system. The gRNAs comprise a crRNA sequence, which in turn comprises a complementary region (also called a spacer) that recognizes and binds a complementary target DNA of interest. The length of the spacer or complementary region is generally between 15 and 30 nucleotides, usually about 20 nucleotides in length, although will vary based on the requirements of the specific CRISPR/Cas system. In some embodiments, the spacer or complementary region is fully complementary to the target DNA sequence. In other embodiments, the spacer is partially complementary to the target DNA sequence, for example at least 80%, 85%, 90%, 95%, 98%, or 99% complementary.

In some embodiments, the gRNAs provided herein further comprise a tracrRNA sequence, which comprises a scaffold region for binding to a nuclease. The length and/or sequence of the tracrRNA may vary depending on the specific nuclease being used for editing. In some embodiments, nuclease binding by the gRNA does not require a tracrRNA sequence. In those embodiments where the gRNA comprises a tracrRNA, the crRNA sequence may further comprise a repeat region for hybridization with complementary sequences of the tracrRNA.

In some embodiments, the gRNAs provided herein comprise two or more gRNA molecules, for example, a crRNA and a tracrRNA, as two separate molecules. In other embodiments, the gRNAs are single guide RNAs (sgRNAs), including sgRNAs comprising a crRNA and a tracrRNA on a single RNA molecule. In some of these embodiments, the crRNA and tracrRNA are linked by an intervening tetraloop.

In some embodiments, one gRNA is used in association with a site-directed nuclease for targeted editing of a gene locus of interest. In other embodiments, two or more gRNAs targeting the same gene locus of interest are used in association with a she-directed nuclease.

In some embodiments, exemplary gRNAs (e.g., sgRNAs) for use with various common Cas nucleases that require both a crRNA and tracrRNA, including Cas9 and Cas12b (C2c1), are provided in Table 31. See, e.g., Jinek et al., Science (2012) 337 (6096):816-821; Dang et al., Genome Biology (2015) 16:280; Ran et al., Nature (2015) 520:186-191; Strecker et al., Nature Comm. (2019) 10:212. For each exemplary gRNA, sequences for different portions of the gRNA, including the complementary region or spacer, crRNA repeat region, tetraloop, and tracrRNA, are shown. In some embodiments, the gRNA comprises all or a portion of the nucleotide sequences set forth In SEQ ID NOs: 179-182. In some embodiments, the gRNA comprises all or a portion of the nucleotide sequences set forth in SEQ ID NOs: 183-186. In some embodiments, the gRNA comprises all or a portion of the nucleotide sequences set forth in SEQ ID NOs187-190. In some embodiments, the gRNA comprises all or a portion of the nucleotide sequences set forth in SEQ ID NOs: 191-194.

In some embodiments, the gRNA comprises a crRNA repeat region comprising, consisting of, or consisting essentially of the nucleotide sequence set forth in SEQ ID NO: 180, SEQ ID NO: 184, SEQ ID NO: 188, or SEQ ID NO: 193. In some embodiments, the gRNA comprises a tetraloop comprising, consisting of, or consisting essentially of the nucleotide sequence set forth in SEQ ID NO: 181 or SEQ ID NO: 192. In some embodiments, the gRNA comprises a tracrRNA comprising, consisting of, or consisting essentially of the nucleotide sequence set forth in SEQ ID NO: 182, SEQ ID NO: 188, SEQ ID NO: 190, or SEQ ID NO: 191.

In some embodiments, the gRNA comprises a complementary region specific to a target gene locus of interest, for example, the TRAC locus, the TRBC1 locus, the TRBC2 locus, B2M locus, the CIITA locus, or a safe harbor locus selected from the group consisting of an AAVS1, ABO, CCR5, CLYBL, CXCR4, F3, FUT1, HMGB1, KDM5D, LRP1, MICA, MICB, RHD, ROSA26, and SHS231 gene locus. The complementary region may bind a sequence in any region of the target gene locus, including for example, a CDS, an exon, an intron, a sequence spanning a portion of to an exon and a portion of an adjacent intron, or a regulatory region (e.g., promoter, enhancer). Where the target sequence is a CDS, exon, intron, or sequence spanning portions of an exon and intron, the CDS, exon, intron, or exon/intron boundary are defined according to any splice variant of the target gene. In some embodiments, the genomic locus targeted by the gRNA is located within 4000 bp, within 3500 bp, within 3000 bp, within 2500 bp, within 2000 bp, within 1500 bp, within 1000 bp, or within 500 bp of any of the loci or regions thereof as described. Further provided herein are compositions comprising one or more gRNAs provided herein and a Cas protein or a nucleotide sequence encoding a Cas protein. In some of these embodiments, the one or more gRNAs and a nucleotide sequence encoding a Cas protein are comprised within a vector, for example, a viral vector.

In some embodiments, provided are methods of identifying new loci and/or gRNA sequences for use in the site-directed genomic insertion approaches as described. For example, for CRISPR/Cas systems, when an existing gRNA for a particular locus (e.g., within an endogenous TCR gene locus) is known, an “inch worming” approach is used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus are between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide is designed according to the sequence of that locus for use in site-directed insertion of transgenes. Although the CRISPR/Cas system is described as illustrative, in some embodiments, any gene editing approach as described is used in this method of identifying new loci, including those using ZFNs, TALENs, meganucleases, and transposases.

In some embodiments, the activity, stability, and/or other characteristics of gRNAs are altered through the incorporation of chemical and/or sequential modifications. As one example, transiently expressed or delivered nucleic acids are prone to degradation by, e.g., cellular nucleases. Accordingly, the gRNAs described herein can contain one or more modified nucleosides or nucleotides which introduce stability toward nucleases. While not being bound by a particular theory, it is believed that some modified gRNAs described herein can exhibit a reduced innate immune response when introduced into a population of cells, particularly the cells of the present technology. As used herein, the term “innate immune response” includes a cellular response to exogenous nucleic acids, including single stranded nucleic acids, generally of viral or bacterial origin, which involves the induction of cytokine expression and release, particularly the interferons, and cell death. Other common chemical modifications of gRNAs to improve stabilities, increase nuclease resistance, and/or reduce immune response include 2′-O-methyl modification, 2′-fluoro modification, 2′-O-methyl phosphorothioate linkage modification, and 2′-O-methyl 3′ thioPACE modification.

One common 3′end modification is the addition of a poly(A) tract comprising one or more (and typically 5-200) adenine (A) residues. In some embodiments, the poly(A) tract is contained in the nucleic acid sequence encoding the gRNA or is added to the gRNA during chemical synthesis, or following in vitro transcription using a polyadenosine polymerase (e.g., E. coli poly(A) polymerase). In vivo, poly(A) tracts is added to sequences transcribed from DNA vectors through the use of polyadenylation signals. Examples of such signals are provided in Maeder. Other suitable gRNA modifications include, without limitations, those described in U.S. Patent Application No. US 201710073674 Al and International Publication No. WO 2017/165862 A1, the entire contents of each of which are incorporated by reference herein.

In some embodiments, a tool for designing a gRNA as disclosed herein comprises: Benchling, Broad Institute GPP, CasOFFinder, CHOPCHOP, CRISPick, CRISPOR, Deskgen, E-CRISP, Geneious, Guides, Horizon Discovery. IDT, Off-Spotter, Synthego, or TrueDesign (ThermoFisher). One of ordinary skill in the art would understand that a tool that predicts both activity and specificity (e.g., to limit off-target modification) would be useful for designing a gRNA in some instances as disclosed herein.

F. Delivery of Gene Editing Systems into a Host Cell

In some embodiments, provided are compositions comprising one or more components of a gene editing system described herein, including one or more gRNAs, a site-directed nuclease (e.g., a Cas nuclease) or a nucleotide sequence encoding a site-directed nuclease protein, and a transgene for targeted insertion. In some embodiments, the compositions are formulated for delivery into a cell.

In some embodiments, components of a gene editing system provided herein, including one or more gRNAs, a site-directed nuclease (e.g., a Cas nuclease) or a nucleotide sequence encoding a site-directed nuclease protein, and a transgene (e.g., the first transgene encoding a tolerogenic factor and/or the second transgene encoding a CAR) for targeted insertion, are delivered into a cell in the form of a delivery vector. The delivery vector is any type of vector suitable for introduction of nucleotide sequences into a cell, including, for example, plasmids, adenoviral vectors, adeno-associated viral (AAV) vectors, retroviral vectors, lentiviral vectors, phages, and HDR-based donor vectors. The different components are introduced into a cell together or separately, and, In some embodiments, are delivered In a single vector or multiple vectors.

In some embodiments, the delivery vector is introduced into a cell by any known method in the field, including, for example, viral transformation, calcium phosphate transfection, lipid-mediated transfection, DEAE-dextran, electroporation, microinjection, nucleoporation, liposomes, nanoparticles, or other methods.

In some embodiments, the present technology provides compositions comprising a delivery vector according to various embodiments disclosed herein. In some embodiments, the compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient is a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, Immunogenicity, or any other complication that excessively outweighs its therapeutic benefits. Suitable excipients include water, saline, dextrose, glycerol, or the like and combinations thereof. In some embodiments, compositions comprising cells as disclosed herein further comprise a suitable infusion media.

In some embodiments, provided are cells or compositions thereof comprising one or more components of a gene editing system described herein, including one or more gRNAs, a site-directed nuclease (e.g., a Cas nuclease) or a nucleotide sequence encoding a site-directed nuclease protein, and a transgene for targeted insertion.

### Methods of Treatment

In some aspects, the present technology provides methods for treating and/or preventing a disease in a subject in need thereof using T cells, such as immune evasive allogeneic T cells, derived from or generated by methods according to various embodiments disclosed herein. The method entails administering to the subject a therapeutically effective amount of the T cell, or a pharmaceutical composition containing the same.

In some embodiments, the T cell is an autologous cell, i.e., obtained from the subject who will receive the T cell after modification. In some embodiments, the T cell is an allogeneic T cell, i.e., obtained from someone other than the subject who will receive the T cell after modification. In either of these embodiments, the T cells is primary T cells obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the T cells is derived from ESCs or iPSCs.

In some embodiments, the T cell is a naive T cell, a helper T cell (CD4+), a cytotoxic T cell (CD8+), a regulatory T cell (Treg), a central memory T cell (TCM), an effector memory T cell (TEM), a stem cell memory T cell (TSCM), or any combination thereof. In some embodiments, the T cell expresses a tolerogenic factor (e.g., CD47, HLA-E, HLA-G, PD-L1, CTLA-4) and/or a CAR (e.g., CD19 CAR). In these embodiments, the T cell recognizes and initiates an immune response to a cell expressing the antigen the CAR is designed to target (e.g., CD19), and the T cell possesses hypoimmunity in an allogeneic recipient due to expression of the tolerogenic factor.

In some embodiments, the present technology provides methods for treating and/or preventing a disease in a subject in need thereof using viral vectors, such as a viral vector comprising a polynucleotide encoding a chimeric antigen receptor, for example, a viral vector generated by methods according to various embodiments disclosed herein. The method entails administering to the subject a therapeutically effective amount of the viral vector, or a pharmaceutical composition containing the same.

In some embodiments, the present technology provides methods for treating and/or preventing a disease in a subject in need thereof using viral vectors, such as a lipid particle comprising a polynucleotide encoding a chimeric antigen receptor, for example, a viral vector generated by methods according to various embodiments disclosed herein. The method entails administering to the subject a therapeutically effective amount of the lipid particle, or a pharmaceutical composition containing the same.

In some embodiments, the disease is cancer, for example, one associated with CD19 expression, i.e., the cancer cell expresses CD19. In these embodiments, the method comprises contacting the cancer cell with a T cell generated by methods of the present technology and expressing the corresponding CAR, such that the CAR is activated in response to the antigen expressed on the cancer cell and subsequently initiates killing of the cancer cell.

In some embodiments, the cancer is a hematologic malignancy. Non-limiting examples of hematologic malignancies include myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL). T-cell acute lymphoid leukemia (T-ALL), multiple myeloma (MM), T-cell lymphoma, and B-cell lymphoma.

In some embodiments, a cancer is solid malignancy. Non-limiting examples of solid malignancies comprise; breast cancer, ovarian cancer, colon cancer, prostate cancer, epithelial cancer, renal-cell carcinoma, pancreatic adenocarcinoma, cervical carcinoma, colorectal cancer, glioblastoma, rhabdomyosarcoma, neuroblastoma, melanoma, Ewing sarcoma, osteosarcoma, mesothelioma and adenocarcinoma.

In some embodiments, the disease is an autoimmune disease, including, for example, lupus, systemic lupus erythematosus, lupus nephritis, extrarenal lupus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, vasculitis, ANCA-vasculitis, and celiac disease.

In some embodiments, the disease is diabetes mellitus, including, for example, Type I diabetes, Type II diabetes, prediabetes, and gestational diabetes.

In some embodiments, the disease is a neurological disease, including, for example, catalepsy, epilepsy, encephalitis, meningitis, migraine, Huntington's, Alzheimer's, Parkinson's, Pelizaeus-Merzbacher disease, and multiple sclerosis.

### A. Compositions, Formulations, and Dosage Regimens

Provided herein are compositions suitable for use in a subject, including therapeutic compositions and cell therapy compositions. Provided herein are pharmaceutical compositions comprising a population of engineered cells as described herein and a pharmaceutically acceptable additive, carrier, diluent or excipient. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); salts such as sodium chloride; and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline). In some embodiments, the pharmaceutically acceptable additive, carrier, diluent or excipient comprises one or more of Plasma-Lyte A®, dextrose, dextran, sodium chloride, human serum albumin (HSA), dimethylsulfoxide (DMSO), or a combination thereof. In some embodiments, the composition further comprises a pharmaceutically acceptable buffer. In some embodiments, the pharmaceutically acceptable buffer is neutral buffer saline or phosphate buffered saline.

In some embodiments, the T cell, or a pharmaceutical composition containing the same, according to the present technology is administered in a manner appropriate to the disease, condition, or disorder to be treated as determined by persons skilled in the medical art. In any of the above embodiments, the T cell, or a pharmaceutical composition containing the same, is administered intravenously, intraperitoneally, intratumorally, into the bone marrow, into a lymph node, or into the cerebrospinal fluid, so as to encounter the target antigen or cells. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In some embodiments, the amount of the T cells in a pharmaceutical composition is typically greater than 10² cells, for example, about 1×10², 5×10², 1×10³, 5×10³, 1×10⁴, 5×10⁴, 1×10⁵, 5×10⁶, 1×10⁷, 1×10⁶, 1×10⁷, 5×10⁷, 1×10⁸, 5×10⁸, ×10⁹, 5×10⁹, 1×10¹⁰, 5×10¹⁰ cells, or more.

In some embodiments, the methods comprise administering to the subject the T cell, or a pharmaceutical composition containing the same, once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. In some embodiments, the host cells or the pharmaceutical composition containing the same is administered every day, every other day, every third day, weekly, biweekly (i.e., every other week), every third week, monthly, every other month, or every third month.

In some embodiments, the T cell, or a pharmaceutical composition containing the same, is administered over a pre-determined time period. Alternatively, the T cell, or a pharmaceutical composition containing the same, is administered until a particular therapeutic benchmark is reached. In some embodiments, the methods provided herein include a step of evaluating one or more therapeutic benchmarks in a biological sample, such as, but not limited to, the level of a cancer biomarker, to determine whether to continue administration of the host cell, or the pharmaceutical composition containing the same.

Provided herein are compositions containing the lipid particles that are derived from virus, such as viral particles or virus-like particles, including those derived from retroviruses or lentiviruses, including pseudotyped lipid particles containing a retargeted attachment protein comprising (i) a paramyxovirus envelope attachment protein; (ii) a targeting moiety directed to a first target molecule expressed on the surface of a target cell, and at least one paramyxovirus fusion protein, and (iii) a polynucleotide encoding any of the chimeric antigen receptors described herein. The pharmaceutical compositions can include any of the described lipid particles.

Also provided herein are compositions comprising any of the lipid particles described herein (e.g., lipid particles that are derived from virus, such as viral particles or virus-like particles, including those derived from retroviruses or lentiviruses).

Also provided herein are compositions containing the lipid particles herein(e.g., lipid particles that are derived from virus, such as viral particles or virus-like particles, including those derived from retroviruses or lentiviruses), including lipid particles containing a retargeted attachment protein, comprising: (a) a first paramyxovirus envelope attachment protein; and a first targeting moiety directed to a target molecule expressed on the surface of a target cell; (b) a second paramyxovirus envelope attachment protein; and a second targeting moiety directed to a target molecule expressed on the surface of a target cell; (c) at least one paramyxovirus fusion protein; and (d) and a polynucleotide encoding any of the chimeric antigen receptors described herein.

Also provided herein are compositions containing the lipid particles herein (e.g., lipid particles that are derived from virus, such as viral particles or virus-like particles, including those derived from retroviruses or lentiviruses), including lipid particles containing a retargeted attachment protein, comprising: (a) a first paramyxovirus envelope attachment protein; and a first targeting moiety directed to a target molecule expressed on the surface of a target cell; (b) a second paramyxovirus envelope attachment protein; and a second targeting moiety directed to a target molecule expressed on the surface of a target cell; (c) a third paramyxovirus envelope attachment protein, wherein the third paramyxovirus envelope attachment protein is a variant paramyxovirus envelope attachment protein comprising one or more mutations to reduce native tropism relative to the wild-type paramyxovirus envelope attachment protein not comprising the one or more mutations; (d) at least one paramyxovirus fusion protein; and (e) and a polynucleotide encoding any of the chimeric antigen receptors described herein.

Also provided herein are compositions containing the lipid particles herein(e.g., lipid particles that are derived from virus, such as viral particles or virus-like particles, including those derived from retroviruses or lentiviruses), including lipid particles containing a retargeted attachment protein comprising: (a) a first paramyxovirus envelope attachment protein; and a first targeting moiety directed to a target molecule expressed on the surface of a target cell; (b) a second paramyxovirus envelope attachment protein; and a second targeting moiety directed to a target molecule expressed on the surface of a target cell; (c) a third paramyxovirus envelope attachment protein, wherein the third paramyxovirus envelope attachment protein is a variant paramyxovirus envelope attachment protein comprising one or more mutations to reduce native tropism relative to the wild-type paramyxovirus envelope attachment protein not comprising the one or more mutations; (d) at least one paramyxovirus fusion protein; and (e) and a polynucleotide encoding any of the chimeric antigen receptors described herein, and optionally one or more additional paramyxovirus envelope attachment proteins and one or more additional targeting moieties directed to a target molecule expressed on the surface of a target cell.

The pharmaceutical compositions provided herein can include any of the described lipid particles (e.g., lipid particles that are derived from virus, such as viral particles or virus-like particles, including those derived from retroviruses or lentiviruses).

The present disclosure also provides, in some aspects, a pharmaceutical composition comprising the composition described herein and pharmaceutically acceptable carrier.

In some aspects, the choice of carrier is determined in part by the particular lipid particle (e.g., lipid particles that are derived from virus, such as viral particles or virus-like particles, including those derived from retroviruses or lentiviruses)and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA: sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the lipid particle (e.g., lipid particle that is derived from virus, such as viral particles or virus-like particles, including those derived from retroviruses or lentiviruses)meets a pharmaceutical or good manufacturing practices (GMP) standard. In some embodiments, the lipid particle is made according to good manufacturing practices (GMP). In some embodiments, the lipid particle has a pathogen level below a predetermined reference value, e.g., is substantially free of pathogens. In some embodiments, the lipid particle has a contaminant level below a predetermined reference value, e.g., is substantially free of contaminants. In some embodiments, the lipid particle has low immunogenicity.

In some embodiments, formulations of the pharmaceutical compositions described herein may be prepared by any method known or hereafter developed in the art of pharmacology. In some embodiments, preparatory methods include the step of bringing the active ingredient into association with a carrier or one or more other accessory ingredients, and then, if necessary or desirable, shaping or packaging the product into a desired single- or multi-dose unit.

In some embodiments, a “unit dose” is a discrete amount of the pharmaceutical composition comprising a predetermined amount of the active ingredient. In some embodiments, the amount of the active ingredient is generally equal to the dosage of the active ingredient that would be administered to a subject or a convenient fraction of such a dosage such as, for example, one-half or one-third of such a dosage. In some embodiments, the unit dosage form may be for a single daily dose or one of multiple daily doses (e.g., about 1 to 4 or more times per day). In some embodiments, when multiple daily doses are used, the unit dosage form may be the same or different for each dose.

In some embodiments, the lipid particle (e.g., lipid particle that is derived from virus, such as viral particles or virus-like particles, including those derived from retroviruses or lentiviruses) containing the variant NiV-G is a viral vector or virus-like particle (e.g.. Section 1II). In some embodiments, the compositions provided herein can be formulated in dosage units of genome copies (GC). Suitable method for determining GC have been described and include. e.g., qPCR or digital droplet PCR (ddPCR) as described in, e.g.. M. Lock et al, Hu Gene Therapy Methods, Hum Gene Ther Methods 25(2):115-25. 2014, which is incorporated herein by reference. In some embodiments, the dosage of administration of a viral vector or virus-like particle is from about 104 to about 1010 GC units, inclusive. In some embodiments, the dosage of administration of a viral vector or virus-like particle is from about 109 to about 1015 GC units, inclusive. In some embodiments, the dosage of administration of a viral vector or virus-like particle is from about 105 to about 109 GC units, inclusive. In some embodiments, the dosage of administration of a viral vector or virus-like particle is from about 106 to about 109 GC units, inclusive. In some embodiments, the dosage of administration of a viral vector or virus-like particle is from about 1012 to about 1014 GC units, inclusive. In some embodiments, the dosage of administration is 1.0×109 GC units, 5.0×109 GC units, 1.0×1010 GC units, 5.0×1010 GC units, 1.0×1011 GC units, 5.0×1011 GC units, 1.0×1012 GC units, 5.0×1012 GC units, or 1.0×1013 GC units, 5.0×1013 GC units, 1.0×1014 GC units, 5.0×1014 GC units, or 1.0×1015 GC units.

In some embodiments, the dosage of administration of a viral vector or virus-like particle is from about 10⁴ to about 10¹⁰ infectious units, inclusive. In some embodiments, the dosage of administration of a viral vector or virus-like particle is from about 10⁹ to about 10¹⁵ infectious units, inclusive. In some embodiments, the dosage of administration of a viral vector or virus-like particle is from about 10⁵ to about 10⁹ infectious units. In some embodiments, the dosage of administration of a viral vector or virus-like particle is from about 10⁸ to about 10⁹ infectious units. In some embodiments, the dosage of administration of a viral vector or virus-like particle is from about 10¹² to about 10¹⁴ infectious units, inclusive. In some embodiments, the dosage of administration is 1.0×10⁹ infectious units, 5.0×10⁹ infectious units, 1.0×10¹⁰ infectious units, 5.0×10¹⁰ infectious units. 1.0×10¹¹ infectious units, 5.0×10¹¹ infectious units, 1.0×10¹² infectious units, 5.0×10¹² infectious units, or 1.0×10¹3 infectious units, 5.0×10¹³ infectious units, 1.0×10′⁴ infectious units, 5.0×10¹⁴ infectious units, or 1.0×10'S infectious units. The techniques available for quantifying infectious units are routine in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodies or by plaque assay.

In some embodiments, methods that calculate the Infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID50) method, which determines the dilution of virus at which 50% of the cell cultures are infected and hence, generally, can determine the titer within a certain range, such as one log.

In some embodiments, the dosage of administration of a viral vector or virus-like particle is from about 10⁴ to about 10¹⁰ plaque forming units (pfu), inclusive. In some embodiments, the dosage of administration of a viral vector or virus-like particle is from about 10⁹ to about 10¹⁵ pfu, inclusive. In some embodiments, the dosage of administration of a viral vector or virus-like particle is from about 10⁵ to about 10⁹ pfu. In some embodiments, the dosage of administration of a viral vector or virus-like particle is from about 10⁶ to about 10⁹ pfu. In some embodiments, the dosage of administration of a viral vector or virus-like particle is from about 10¹² to about 10¹⁴ pfu, inclusive. In some embodiments, the dosage of administration is 1.0×10⁹ pfu, 5.0×10⁹ pfu, 1.0×10¹⁰ pfu, 5.0×10¹⁰ pfu, 1.0×10¹¹ pfu, 5.0×10¹¹ pfu, 10×10¹² pfu, 5.0×10¹² pfu, or 1.0×10¹³ pfu. 5.0×10¹³ pfu, 1.0×10¹⁴ pfu, 5.0×10¹⁴ pfu, or 1.0×10¹⁵ pfu.

In some embodiments, the subject will receive a single injection. In some embodiments, administration can be repeated at daily/weekly/monthly intervals for an indefinite period and/or until the efficacy of the treatment has been established. As set forth herein, the efficacy of treatment can be determined by evaluating the symptoms and clinical parameters described herein and/or by detecting a desired response.

The exact amount of vehicle provided lipid particle (e.g., lipid particles that are derived from virus, such as viral particles or virus-like particles, including those derived from retroviruses or lentiviruses) required will vary from subject to subject, depending on the species, age, weight and general condition of the subject, the particular polynucleic acid, polypeptide, or vector used, its mode of administration etc. TAn appropriate amount can be determined by one of ordinary skill in the art using only routine experimentation given the teachings herein.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the lipid particles (e.g., lipid particles that are derived from virus, such as viral particles or virus-like particles, including those derived from retroviruses or lentiviruses) in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g., films, or microcapsules.

In some embodiments, vehicle formulations may comprise cyroprotectants. As used herein, there term “cryoprotectant” refers to one or more agent that when combined with a given substance, helps to reduce or eliminate damage to that substance that occurs upon freezing. In some embodiments, cryoprotectants are combined with vector vehicles in order to stabilize them during freezing. In some aspects, Frozen storage of RNA between −20° C. and −80° C. may be advantageous for long term (e.g., 36 months) stability of polynucleotide. In some embodiments, the RNA species is mRNA. In some embodiments, cryoprotectants are included in vehicle formulations to stabilize polynucleotide through freeze/thaw cycles and under frozen storage conditions. Cryoprotectants of the provided embodiments may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

The formulations to be used for in vivo administration are generally sterile. Sterility may be readily accomplished, e.g., by filtration through sterile filtration membranes.

In some embodiments, the method further entails administering one or more other cancer therapies such as surgery, immunotherapy, radiotherapy, and/or chemotherapy to the subject, sequentially or simultaneously.

In some embodiments, the methods further comprise administering the subject a pharmaceutically effective amount of one or more additional therapeutic agents to obtain improved or synergistic therapeutic effects. In some embodiments, the one or more additional therapeutic agents are selected from the group consisting of an immunotherapy agent, a chemotherapy agent, and a biologic agent. In some embodiments, the subject was administered the one or more additional therapeutic agents before administration of the T cell, or a pharmaceutical composition containing the same. In some embodiments, the subject is co-administered the one or more additional therapeutic agents and the T cell, or a pharmaceutical composition containing the same. In some embodiments, the subject was administered the one or more additional therapeutic agents after administration of the T cell, or a pharmaceutical composition containing the same.

As one of ordinary skill in the art would understand, the one or more additional therapeutic agents and the T cell, or a pharmaceutical composition containing the same, is administered to a subject in need thereof one or more times at the same or different doses, depending on the diagnosis and prognosis of the subject. One skilled in the art would be able to combine one or more of these therapies in different orders to achieve the desired therapeutic results. In some embodiments, the combinational therapy achieves improved or synergistic effects in comparison to any of the treatments administered alone.

B. Methods for Administering Hypoimmunogenic Cells Including T Cells As is described in further detail herein, provided herein are methods for treating a patient with a condition, disorder, or disorder through administration of hypoimmunogenic cells, particularly hypoimmunogenic T cells. As will be appreciated, for all the multiple embodiments described herein related to the timing and/or combinations of therapies, the administration of the cells is accomplished by a method or route which results in at least partial localization of the introduced cells at a desired site. The cells can be infused, implanted, or transplanted directly to the desired site, or alternatively be administered by any appropriate route which results in delivery to a desired location in the subject where at least a portion of the implanted cells or components of the cells remain viable.

Provided herein are methods for treating a patient with a condition, disorder, or disorder includes administration of a population of hypoimmunogenic cells (e.g., primary T cells, T cells differentiated from hypoimmunogenic induced pluripotent stem cells, or other cells differentiated from hypoimmunogenic induced pluripotent stem cells described herein) to a subject, e.g., a human patient. For instance, a population of hypoimmunogenic primary T cells such as, but limited to, CD3+ T cells, CD4+ T cells, CD8+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells that express CD45RA (TEMRA cells), tissue-resident memory (Trm) cells, virtual memory T cells, innate memory T cells, memory stem cell (Tsc), γδ T cells, and any other subtype of T cell is administered to a patient to treat a condition, disorder, or disorder. In some embodiments, an immunosuppressive and/or immunomodulatory agent (such as, but not limited to a lymphodepletion agent) is not administered to the patient before the administration of the population of hypoimmunogenic cells. In some embodiments, an immunosuppressive and/or immunomodulatory agent is administered at least 1, 2, 3, 4. 5, 6, 7, 8, 9, 10, 11, 12, 13, 14 days or more before the administration of the cells. In some embodiments, an immunosuppressive and/or immunomodulatory agent is administered at least 1 week, 2 weeks, 3 weeks, 4 weeks, 5 weeks, 6 weeks, 7 weeks. 8 weeks, 9 weeks. 10 weeks or more before the administration of the cells. In numerous embodiments, an immunosuppressive and/or immunomodulatory agent is not administered to the patient after the administration of the cells, or is administered at least 1. 2, 3, 4, 5, 6, 7, 8, 9, 10. 11, 12, 13. 14 days or more after the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the administration of the cells. In some embodiments where an immunosuppressive and/or immunomodulatory agent is administered to the patient before or after the administration of the cells, the administration is at a lower dosage than would be required for cells with one or more MHC I and/or MHC II molecule expression and without exogenous expression of CD47.

Non-limiting examples of an immunosuppressive and/or immunomodulatory agent (such as, but not limited to a lymphodepletion agent) include cyclosporine, azathioprine, mycophenolic acid, mycophenotate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-a and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, or CD58, and antibodies binding to any of their ligands. In some embodiments, such an immunosuppressive and/or immunomodulatory agent may be selected from soluble IL-15R, IL-10, B7 molecules (e.g., B7-1, B7-2, variants thereof, and fragments thereof), ICOS, and OX40, an inhibitor of a negative T cell regulator (such as an antibody against CTLA-4) and similar agents.

In some embodiments, where an immunosuppressive and/or immunomodulatory agent is administered to the patient before or after the administration of the cells, the administration is at a lower dosage than would be required for cells with one or more MHC I and/or MHC It molecule expression, TCR expression and without exogenous expression of CD47. In some embodiments, where an immunosuppressive and/or immunomodulatory agent is administered to the patient before or after the first administration of the cells, the administration is at a lower dosage than would be required for cells with one or more MHC I and MHC II molecule expression, TCR expression and without exogenous expression of CD47.

In some embodiments, the cells described are co-administered with a therapeutic agent that that binds to and/or interacts with one or more receptors selected from the group consisting of CD94. KIR2DL4, PD-1, an inhibitory NK cell receptor, and an activating NK receptor. In some instances, the therapeutic agent binds to a receptor on the surface of an NK cell, including one or more subpopulations of NK cells. In some embodiments, the therapeutic agent is selected from the group consisting of an antibody and fragments and variants thereof, an antibody mimetic, a small molecule, a blocking peptide, and a receptor antagonist.

For therapeutic application, cells prepared according to the disclosed methods can typically be supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

In some embodiments, the cells described herein are contraindicated in patients with known Type I hypersensitivity or anaphylactic reactions to murine proteins, Chinese Hamster Ovary (CHO) cell proteins, or to any component of the compositions described herein. In some embodiments, the cells described herein are contraindicated in patients who have or have had progressive multifocal leukoencephalopathy (PML). In some embodiments, the cells described herein are not recommended for use in patients with severe, active infections.

In some embodiments, the cells described herein are administered to a subject with an autoimmune disease/disorder and/or inflammatory disease/disorder who has been previously treated with rituximab (RITUXAN®). In some embodiments, the cells described herein are administered to a subject with an autoimmune disease/disorder and/or inflammatory disease/disorder who has been previously treated with rituximab (RITUXAN®) and has failed and/or not responded to the rituximab treatment. In some embodiments, the patent has rheumatoid arthritis (RA). In some embodiments, the patient has RA and the rituximab treatment is in combination with methotrexate. In some embodiments, the patient is an adult patient that has moderately-to severely-active RA. In some embodiments, the patient is an adult patient that has moderately-to severely-active RA and the rituximab treatment is in combination with methotrexate. In some embodiments, the patient is an adult patient that has moderately-to severely-active RA who has inadequate response to one or more TNF antagonist therapies and the rituximab treatment is in combination with methotrexate. In some embodiments, the rituximab dose for RA in combination with methotrexate is two-1000 mg intravenous infusions separated by 2 weeks (one course) every 24 weeks and/or based on clinical evaluation, but not sooner than every 16 weeks. In some embodiments, the Methylprednisolone 100 mg intravenous or equivalent glucocorticoid is recommended 30 minutes prior to each infusion.

In some embodiments, the cells described herein are administered to a subject with an autoimmune disease/disorder and/or inflammatory disease/disorder who has been previously treated with rituximab (RITUXAN®). In some embodiments, the cells described herein are administered to a subject with an autoimmune disease/disorder and/or inflammatory disease/disorder who has been previously treated with rituximab (RITUXAN®) and has failed and/or not responded to the rituximab treatment. In some embodiments, the patent has granulomatosis with polyangiitis (GPA) (Wegener's Granulomatosis). In some embodiments, the patent has Microscopic polyangiitis (MPA) in adult patients in combination with glucocorticoids. In some embodiments, the rituximab dose for GPA and MPA in combination with glucocorticoids is 375 mg/m2 once weekly for 4 weeks. In some embodiments, the rituximab is administered as a 100 mg/10 mL solution in a single-use vial. In some embodiments, the rituximab is administered as a 500 mg/50 mL solution in a single-use vial.

In some embodiments, cells described herein are administered to a subject with an autoimmune disease/disorder and/or inflammatory disease/disorder as part of a combination therapy. In some embodiments, cells described herein are administered to a subject with an autoimmune disease/disorder and/or inflammatory disease/disorder as part of a combination therapy with an anti-B-lymphocyte stimulator (anti-BLyS) therapy. In some embodiments, an anti-BLyS therapy comprises belimumab.

C. Autoimmune Diseases/Disorders and/or Inflammatory Diseases/Disorders for Treatment

Autoimmune or inflammatory disorders include diseases or disorders arising from and directed against an individual's own tissues or organs or a manifestation thereof or a condition resulting therefrom. In one embodiment, it refers to a condition that results from, or is aggravated by, the production of T cells and/or B cells that are reactive with normal body tissues and antigens. In one embodiment, it refers to a condition that results from, or is aggravated by, the production by antibodies that are reactive with normal body tissues and antigens.

In some embodiments, autoimmune or inflammatory disorders include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis (such as plaque psoriasis, gutatte psoriasis, pustular psoriasis, and psoriasis of the nails), atopy (including atopic diseases such as hay fever and Job's syndrome), dermatitis (including contact dermatitis, chronic contact dermatitis, exfoliative dermatitis, exfoliative psoriatic dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis), x-linked hyper IgM syndrome, allergic intraocular inflammatory diseases, urticaria (such as chronic allergic urticaria, chronic idiopathic urticaria, chronic autoimmune urticaria), myositis, polymyositis/dermatomyositis, juvenile dermatomyositis, toxic epidermal necrolysis, scleroderma (including systemic scleroderma), sclerosis (such as systemic sclerosis; multiple sclerosis (MS), MS associated with EBV infection, spino-optical MS, primary progressive MS (PPMS), relapsing-remitting MS (RRMS), progressive relapsing MS, secondary progressive MS (SPMS), progressive systemic sclerosis, atherosclerosis, arteriosclerosis, sclerosis disseminats, and ataxic sclerosis), neuromyelitis optica spectrum disorder (NMO, also known as Devic's Disease or Devic's Syndrome), inflammatory bowel disease (IBD) including Crohn's disease; autoimmune-mediated gastrointestinal diseases: colitis such as ulcerative colitis, colitis ulcerosa, microscopic colitis, collagenous colitis, colitis polyposa, necrotizing enterocolitis, and transmural colitis; and autoimmune inflammatory bowel disease), bowel inflammation, pyoderma gangrenosum, erythema nodosum, primary sclerosing cholangitis, respiratory distress syndrome (including adult or acute respiratory distress syndrome (ARDS)), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition. IgE-mediated diseases such as anaphylaxis and allergic and atopic rhinitis, encephalitis such as Rasmussen's encephalitis and limbic and/or brainstem encephalitis, uveitis (such as anterior uveitis, acute anterior uveitis, granulomatous uveitis, nongranulomatous uveitis, phacoantigenic uveitis, posterior uveitis, or autoimmune uveitis), glomerulonephritis (GN) with and without nephrotic syndrome (such as chronic or acute glomerulonephritis, primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, or proliferative nephritis), autoimmune polyglandular endocrine failure, balanitis including balanitis circumscripta plasmacellularis, balanoposthitis, erythema annulare centrifugum, erythema dyschromicum perstans, erythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema (including allergic or atopic eczema, asteatotic eczema, dyshidrotic eczema, and vesicular palmoplantar eczema), asthma (such as asthma bronchiale, bronchial asthma, and auto-immune asthma), conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus (including lupus nephritis, lupus cerebritis, pediatric lupus, non-renal lupus, extra-renal lupus, discoid lupus and discoid lupus erythematosus, alopecia lupus, systemic lupus erythematosus (SLE), cutaneous SLE or subacute cutaneous SLE, neonatal lupus syndrome (NLE), and lupus erythematosus disseminatus, Type I diabetes. Type II diabetes, and latent autoimmune diabetes in adults (or Type 1.5 diabetes), juvenile onset (Type I) diabetes mellitus, including pediatric insulin-dependent diabetes mellitus (IDDM), adult onset diabetes mellitus (Type i1 diabetes), idiopathic diabetes, insipidus, diabetic retinopathy, diabetic nephropathy, and diabetic large-artery disorder; immune responses associated with acute and delayed hypersensitivity mediated by cytokines and T-lymphocytes, tuberculosis, sarcoidosis, granulomatosis (including lymphomatoid granulomatosis, Wegener's granulomatosis, or agranulocytosis), vasculitides (including vasculitis, large-vessel vasculitis, polymyalgia rheumatica and giant cell (Takayasu's) arteritis, medium-vessel vasculitis, Kawasakrs disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis (such as Churg-Strauss vasculitis or syndrome (CSS) and ANCA-associated small-vessel vasculitis)), temporal arteritis, aplastic anemia, autoimmune aplastic anemia, Coombs positive anemia, Diamond Blackfan anemia, hemolytic anemia, immune hemolytic anemia including autoimmune hemolytic anemia (AIHA), pemicious anemia (anemia pemiciosa), Addison's disease, pure red cell anemia or aplasia (PRCA); Factor VII1 deficiency; hemophilia A; autoimmune neutropenia, pancytopenia, leukopenia, diseases involving leukocyte diapedesis, CNS inflammatory disorders, Alzheimer's disease, Parkinson's disease, multiple organ injury syndrome (such as those secondary to septicemia, trauma, or hemorrhage), antigen-antibody complex-mediated diseases, anti-glomerular basement membrane disease, anti-phospholipid antibody syndrome, anti-phospholipid syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, autoimmune or immune-mediated thrombocytopenia such as idiopathic thrombocytopenic purpura (ITP) including chronic or acute ITP, acquired thrombocytopenic purpura, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases, including thyroiditis autoimmune thyroiditis, Hashimoto's disease, chronic thyroiditis (Hashimoto's thyroiditis), or subacute thyroiditis), autoimmune thyroid disease, idiopathic hypothyroidism, or Grave's disease), polyglandular syndromes, autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), sensory neuropathy, multifocal motor neuropathy, Sheehan's syndrome, hepatitis, including autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome. Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, DressIer's syndrome, alopecia areata, alopecia totalis, CREST syndrome (calcinosis, Raynaud's phenomenon, esophageal dysmotility, sclerodactyly, and telangiectasia), male and female autoimmune infertility (e.g., due to anti-spermatozoan antibodies) mixed connective tissue disease. Chagas' disease, rheumatic fever, recurrent abortion, farmer's lung, erythema multiforme, post-cardiotomy syndrome, post myocardial infarction cardiotomy syndrome, Cushing's syndrome, bird-fanciers lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Samter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cydtis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erytherna nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Lofflers syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy. Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, emphysema, alopecia areata, adipose tissue inflammation/diabetes type 11, obesity associated adipose tissue inflammation/insulin resistance, endometriosis, and pulmonary hemosiderosis.

In some embodiments, the autoimmune disease is multiple sclerosis. In such embodiments, the immune cells, viral vectors, and other compositions containing the antibodies, antigen binding fragments, chimeric antigen receptors, and/or chimeric antigen receptor transgenes described herein are used to treat a patient that is suspected of having multiple sclerosis or has been diagnosed with multiple sclerosis.

In some embodiments, the autoimmune disease is lupus nephritis. In such embodiments, the immune cells, viral vectors, and other compositions containing the antibodies, antigen binding fragments, chimeric antigen receptors, and/or chimeric antigen receptor transgenes described herein are used to treat a patient that is suspected of having lupus nephritis or has been diagnosed with lupus nephritis.

In some embodiments, the autoimmune disease is extrarenal lupus. In such embodiments, the immune cells, viral vectors, and other compositions containing the antibodies, antigen binding fragments, chimeric antigen receptors, and/or chimeric antigen receptor transgenes described herein are used to treat a patient that is suspected of having extrarenal lupus or has been diagnosed with extrarenal lupus.

In some embodiments, the autoimmune disease is antineutrophil cytoplasmic antibody (ANCA)-associated vasculitis. In such embodiments, the immune cells, viral vectors, and other compositions containing the antibodies, antigen binding fragments, chimeric antigen receptors, and/or chimeric antigen receptor transgenes described herein are used to treat a patient that is suspected of having antineutrophil cytoplasmic antibody (ANCA)-associated vasculitis or has been diagnosed with antineutrophil cytoplasmic antibody (ANCA)-associated vasculitis.

i. Multiple Sclerosis

Multiple Sclerosis (MS) is an inflammatory and demyelinating degenerative disease of the human central nervous system (CNS) which affects approximately 300,000 persons in the United States (see, Anderson et al. Ann Neurology 31(3):333-6 (1992); Noonan et al. Neurology 58:136-8 (2002)). MS Is a heterogeneous disorder based on clinical course, magnetic resonance imaging (MRI) scan assessment, and pathology analysis of biopsy and autopsy material (see, Lucchinetti et al. Ann Neurol 47:707-17 (2000)). The disease manifests itself in a large number of possible combinations of deficits, including spinal cord, brainstem, cranial nerve, cerebellar, cerebral, and cognitive syndromes. MS can be difficult to diagnose because of the non-specific clinical findings, which led to the development of highly structured diagnostic criteria that include several technological advances, consisting of MRI scans, evoked potentials, and cerebrospinal fluid (CSF) studies. All diagnostic criteria rely upon the general principles of scattered lesions in the central white matter occurring at different times and not explained by other etiologies such as infection, vascular disorder, or autoimmune disorder (see, McDonald et al. Ann Neurol 50:121-7 (2001)). MS has four patterns of disease: relapsing-remitting MS (RRMS; 80%-85% of cases at onset), primary progressive MS (PPMS; 10%-15% at onset), progressive relapsing MS (PRMS; 5% at onset); and secondary progressive MS (SPMS) (see, Kremenchutzky et al. Brain 122 (Pt 10):1941-50 (1999): Confavreux et al. N Engl J Med 343(20): 1430-8 (2000)). An estimated 50% of patients with RRMS will develop SPMS in 10 years, and up to 90% of RRMS patients will eventually develop SPMS (Weinshenker et al. Brain 112(Pt 1): 133-46 (1989)).

In some embodiments, the multiple sclerosis is relapsing-remitting multiple sclerosis, progressive relapsing multiple sclerosis, primary progressive multiple sclerosis, or secondary progressive multiple sclerosis. In some embodiments, the multiple sclerosis is relapsing-remitting multiple sclerosis. In some embodiments, the multiple sclerosis is progressive relapsing multiple sclerosis. In some embodiments, the multiple sclerosis is primary progressive multiple sclerosis. In some embodiments, the multiple sclerosis is secondary progressive multiple sclerosis.

ii. Lupus Nephritis

Lupus nephritis is a serious and prevalent complication of systemic lupus erythematosus (SLE), a chronic autoimmune disease. In the United States, lupus nephritis affects approximately 40% of individuals with SLE, translating to a significant number of patients given the prevalence of SLE in the general population. The disease is characterized by an autoimmune attack on the kidneys, leading to varying degrees of inflammation and damage. Diagnostic criteria for lupus nephritis primarily include laboratory tests such as urinalysis, which often shows proteinuria and hematuria, and kidney biopsy, which is considered the gold standard for diagnosis. The course of lupus nephritis can be highly variable, ranging from mild to severe forms. Some patients may experience episodic flares, while others progress to chronic kidney disease or end-stage renal disease, necessitating long-term management strategies.

In some embodiments, the lupus nephritis is: class I—minimal nesangial lupus nephritis; class II—mesangial proliferative lupus nephritis; class III— focal lupus nephritis; class IV—diffuse segmental or global lupus nephritis: It's the most severe form and involves more than 50% of the glomeruli; class V—membranous lupus nephritis; and class VI—advanced sclerosing lupus nephritis.

iii. Extrarenal Lupus

Extrarenal lupus, a manifestation of systemic lupus erythematosus (SLE) that affects organs other than the kidneys, is a complex autoimmune condition with significant prevalence in the United States. This form of lupus is characterized by a wide range of clinical manifestations, including but not limited to cutaneous involvement (such as a butterfly rash), arthritis, serositis (inflammation of the linings of the lungs or heart), neurologic symptoms, and hematologic abnormalities. The diagnostic criteria for extrarenal lupus involve a combination of clinical evaluation and laboratory tests, including antinuclear antibody (ANA) testing, and includes an evaluation of eleven criteria established by the American College of Rheumatology for a definitive diagnosis. The course of the disease is highly individualized and can range from mild to life-threatening manifestations. Some patients may experience episodic flares of symptoms interspersed with periods of remission, while others endure a more chronic, persistently active disease course.

iv. Antineutrophil cytoplasmic antibody (ANCA)-associated vasculitis

Antineutrophil cytoplasmic antibody (ANCA)-associated vasculitis is a group of rare autoimmune diseases characterized by inflammation and destruction of small blood vessels. In the United States, AAV affects approximately 3 out of every 100,000 individuals annually. This disease primarily manifests through symptoms such as kidney inflammation, lung hemorrhage, skin lesions, and nerve damage. The diagnosis of AAV is complex and typically involves a combination of clinical assessment, serological tests for anti-neutrophil cytoplasmic antibodies (ANCAs), and tissue biopsy confirming vasculitis. The most common subtypes of AAV include granulomatosis with polyangiitis (GPA), microscopic polyangiltis (MPA), and eosinophilic granulomatosis with polyangiltis (EGPA). The disease course can be highly variable; some patients may experience a single episode followed by remission, while others may have a relapsing-remitting course. Severe cases can lead to critical organ damage and require aggressive immunosuppressive therapy.

### Examples

The present disclosure may be further described by the following non-limiting examples, in which standard techniques known to the skilled artisan and techniques analogous to those described in these examples may be used where appropriate. It is understood that the skilled artisan will envision additional embodiments consistent with the disclosure provided herein.

### Example 1: In Vitro Production of CD19 CAR-T Cells

This example described methods to generate and characterize CAR expression in primary human pan-T cells isolated from donor samples.

Transduction of PanT cells was performed as follows: Primary healthy donor T cells (STEMCELL Technologies), i.e., PanT cells were thawed and activated with CD3/CD8 Dynabeads (Thermo Fisher) at a ratio of 3:1 for 24 hours in the presence of IL-2 and IL-15. Pan-T cells for this experiment came from donor PanT-004 (190981203C), PanT-009 (200380403C), PanT-010 (200381501C), and PanT-012 (2010405012). Once cells were thawed and activated, cells were plated and spinoculated with 375 viral genomes/cell of vesicular stomatitis virus (VSV-G) pseudotyped lentivirus to deliver polynucleotides encoding 4 different CD19 CAR constructs (see Table 32) and a control FMC83 CD19 construct. 72 hours after transduction, the beads were removed, and cells were washed and expanded up to a G-REX plate. Cellular expression of the CD19 CAR constructs was measured by flow cytometric analysis using FITC-labeled human CD19 (Acro Biosystems) on days 4 to 6. All experiments were normalized to live, CAR+ cells. Following confirmation of CAR expression, remaining cells were frozen at −80° C.

### Example 2: In Vitro Characterization of CD19 CAR-T Cells

This example describes methods used to characterize the cytotoxic effects of CD19 CAR-T cells and results of said characterization.

### Flow Cytotoxicity

Flow cytometry-based in vitro characterization of CD19 CAR-T cell cytotoxic effects was performed as follows: Target cells (NALM-6 iRFP713 tumor cells) were counted and plated in a 96-well round bottom plate at 20,000 cells/well. NALM-6 cells were obtained from American Type Culture Collection (ATCC) and maintained in RPMI-1840+10% FBS. For use in in vitro and in vivo functional killing assays, target cell lines were transduced with iRFP713 and flow sorted for purity. Effector cells (T cells from donor PanT-007 transduced with CD19 CAR constructs as disclosed in Table 32 or control FMC63 CD19 CAR construct) were plated in wells with target cells at effector cell:target cell ratios (E:T ratios) of 32:1, 16:1, 8:1, 4:1. 2:1. 1:1, 1:2, 1:4, 1:8, 1:16, and 1:32, normalized to CAR transduction efficiency. After a 24-hour incubation, cells were pelleted and stained for analysis by flow cytometry.

FIGS. 1A and 1B show the results of the flow cytometry-based cytotoxicity characterization of the CD19 CAR-T cells. Notably, CAR 400 (VH-VL) demonstrated more effective tumor killing at E:T ratios approximately 4-fold lower than FMC63 and the other CD19 CARs tested. The enhanced tumor cell killing for CAR 400 (VH-VL) was evident until the E:T ratio of 0.0625, where CAR 400 (VH-VL) exhibited tumor cell killing similar to the other CD19 CAR constructs and the FMC63 benchmark CD19 CAR. CAR 369 (VH-VL), CAR 399 (VL-VH), and CAR 400 (VL-VH) each performed similarly to the FMC63 benchmark CD19 CAR at each tested E:T ratio.

### Live Cell Imaging

In vitro characterization of CD19 CAR-T cytotoxic effects by live cell imaging was performed as follows: Target cells (NALM-6 iRFP713 cells) were counted and plated in a 96-well flat bottom plate at 20,000 cells/well. Effector cells (T cells from donor PanT-007 transduced with CD19 CAR constructs as disclosed in Table 32, control FMC63 CD19 CAR construct, or mock) were plated in wells with target cells at varying effector cell:target cell ratios (E:T ratio) of 1:1, 1:2, 1:4, 1:8, and 1:16. Cells were imaged every 4 hours for 6 days on a Sartorius IncuCyte instrument.

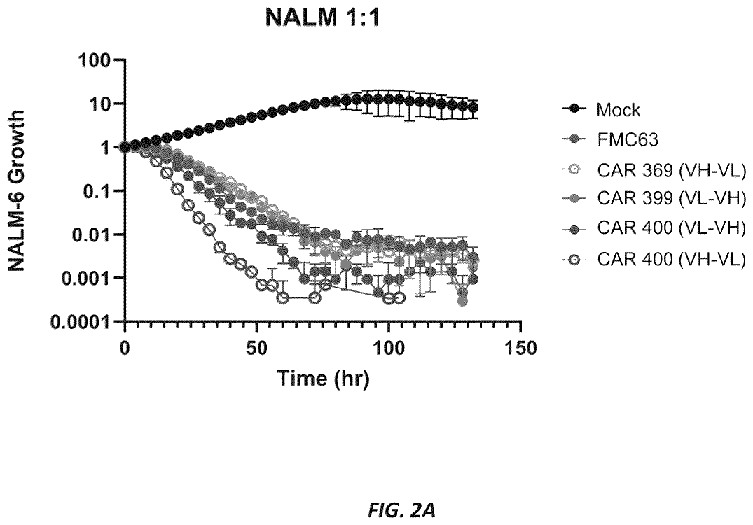

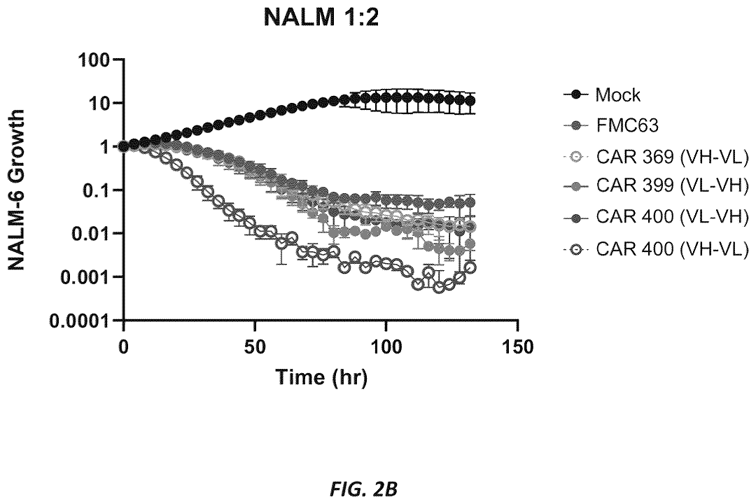

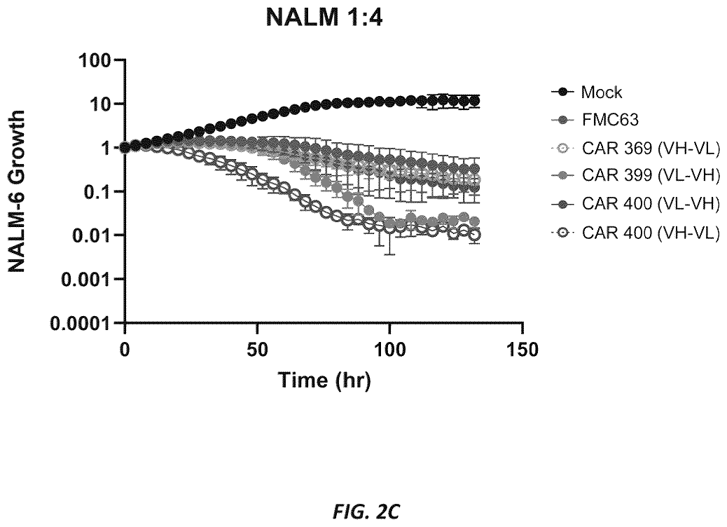

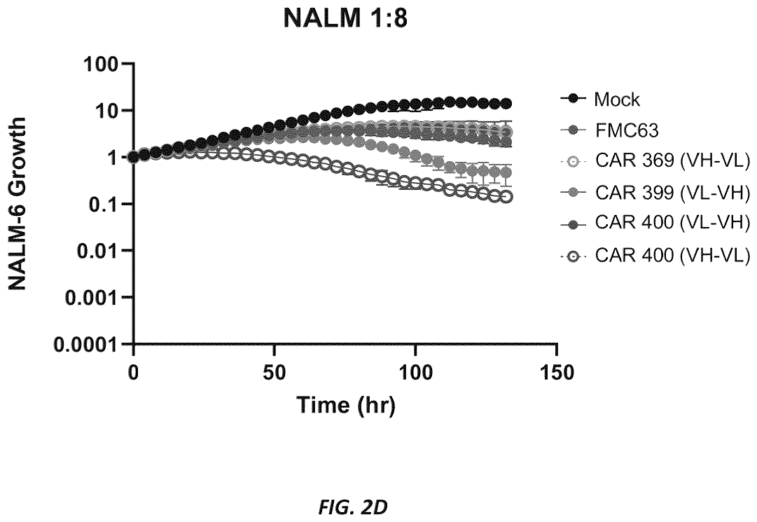

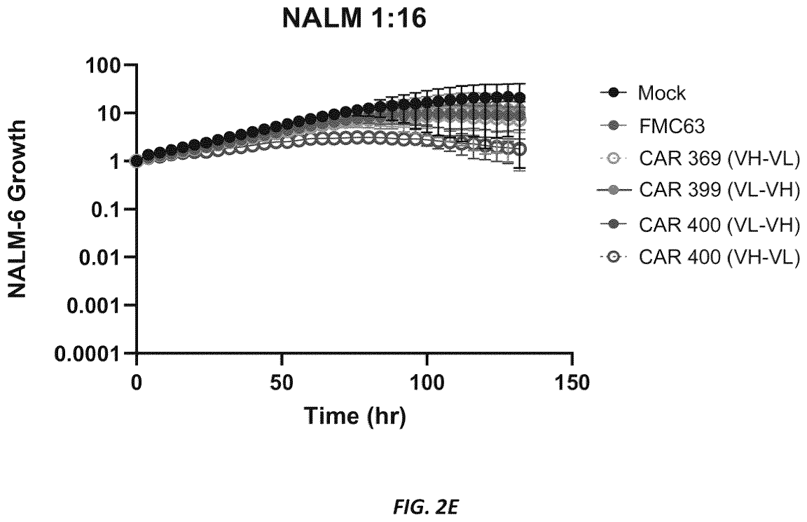

FIGS. 2A-2E show the expansion of NALM-6 iRFP713 cells after culturing with effector cells transduced by CD19 CARs over a 6-day period. Notably, CAR 400 (VH-VL) exhibited tumor cell killing at E:T ratios that were approximately 2-fold lower than the FMC63 benchmark CD19 CAR. FIG. 2A depicts the cytotoxic effect of CDI9 CARs at an E:T ratio of 1:1. At an E:T ratio of 1:1, CAR 400 (VH-VL) exhibited enhanced killing of target NALM-6 iRFP713 cells relative to the other CD19 CAR constructs and the FMC63 benchmark CD19 CAR over the six-day period. CAR 400 (VL-VH) additionally exhibited enhanced tumor cell killing relative to the FMC63 benchmark CD19 CAR. CAR 369 (VH-VL) and CAR 399 (VL-VH) performed similar to the FMC63 benchmark CD19 CAR. FIG. 28 depicts the cytotoxic effect of CD19 CARs at an E:T ratio of 1:2. CAR 400 (VH-VL) exhibited enhanced tumor cell killing relative to all other CD19 CAR constructs tested, including the FMC63 benchmark CD19 CAR over the entire six-day period. The remaining CD19 constructs, CAR 369 (VH-VL), CAR 399 (VL-VH), and CAR 400 (VL-VH) exhibited tumor cell killing similar to the FMC63 benchmark CD19 CAR. FIG. 2C depicts the cytotoxic effect of CD19 CARs at an E:T ratio of 1:4. CAR 400 (VH-VL) exhibited enhanced tumor cell killing relative to all other tested CD19 CARs over the six-day period. While initially exhibiting tumor cell killing similar to the FMC63 benchmark CD19 CAR, CAR 399 (VL-VH) exhibited enhanced tumor cell killing relative to the FMC63 benchmark CAR from approximately 75 hours until the end of the study. CAR 400 (VL-VH) and CAR 369 (VH-VL) exhibited tumor cell killing similar to the FMC63 benchmark CAR. FIG. 2D depicts the cytotoxic effect of CD19 CARs at an E:T ratio of 1:8. CAR 400 (VH-VL) exhibited tumor cell killing that was enhanced relative to all other CD19 CARs tested, including the FMC63 benchmark CD19 CAR. While initially exhibiting tumor cell killing similar to the FMC63 benchmark CD19 CAR, CAR 399 (VL-VH) exhibited enhanced tumor cell killing relative to the FMC63 benchmark CAR from approximately 75 hours until the end of the study. CAR 400 (VL-VH) and CAR 369 (VH-VL) exhibited tumor cell killing similar to the FMC63 benchmark CAR. FIG. 2E depicts the cytotoxic effect of CD19 CARs at an E:T ratio of 1:16. CAR 400 (VH-VL) exhibited tumor cell killing that was enhanced relative to all other CD19 CARs tested, including the FMC63 benchmark CD19 CAR. All other CD19 CARs tested, including the FMC63 benchmark CD19 CAR, exhibited tumor cell killing similar to mock transduced effector cells.

### CD19 Binder Binding

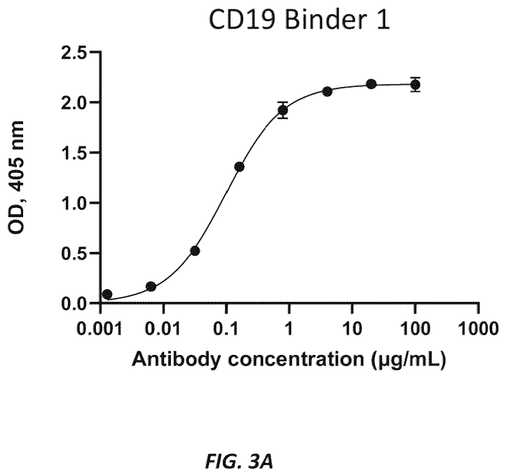

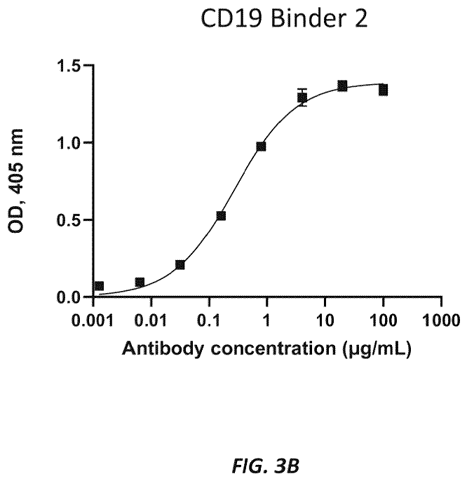

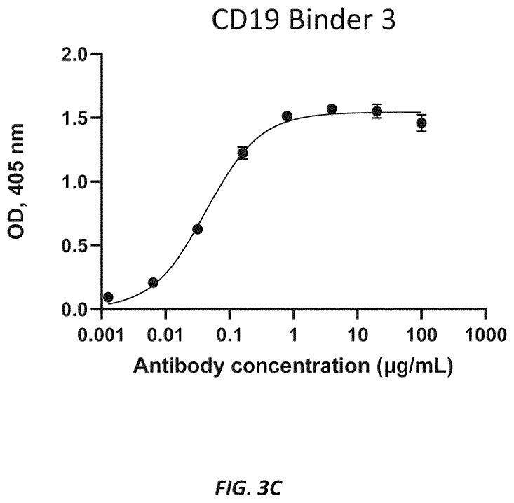

Binding of CD19 Binder 1. 2, and 3 (disclosed in Table 11) to recombinant CD19 was evaluated in an enzyme-linked immunosorbent assay (ELISA). FIGS. 3A-3C illustrate the binding of CD19 binders 1, 2, and 3 to recombinant CD19 as measured by OD at 405 nm. CD19 Binder 1 exhibited an EC₅₀ of 0.099 μg/mL (FIG. 3A). CD19 Binder 2 exhibited an EC₅₀ of 0.27 μg/mL (FIG. 38). CD19 Binder 3exhibited an EC₅₀ of 0.044 μg/mL (FIG. 3C).

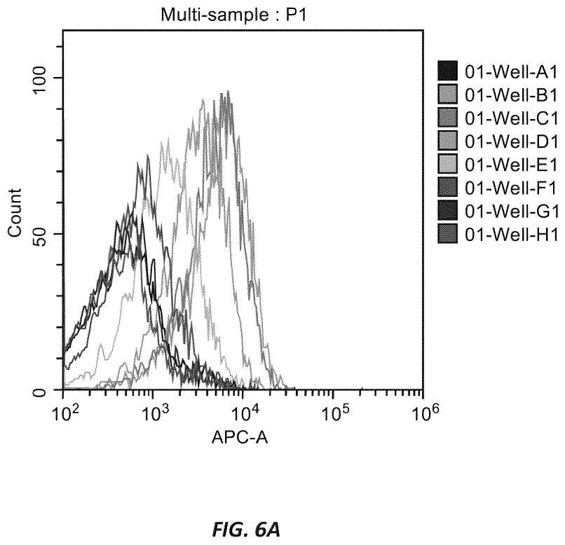

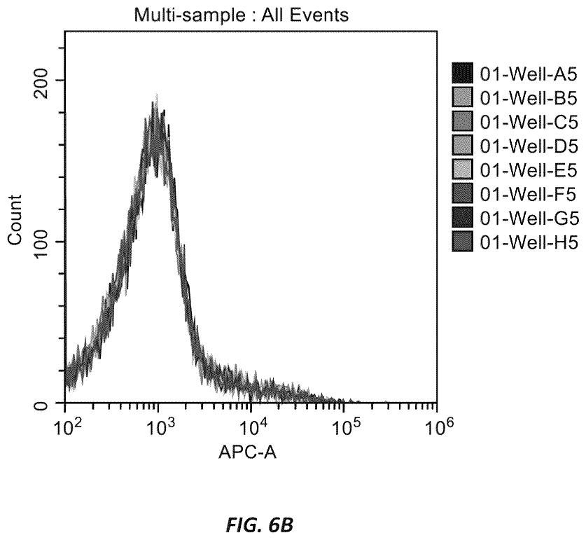

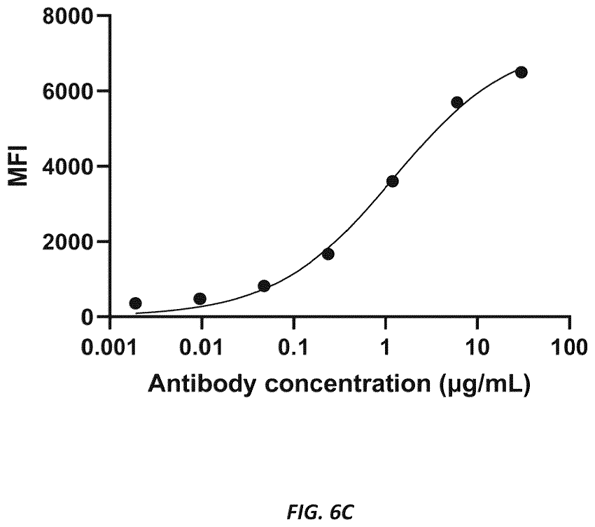

FIGS. 4-6 illustrate the specificity of the humanized CD19 binders to CD19. CD19 Binder 1, 2, and 3 comprise the CD19 binders disclosed in Table 11. CD19 Binder 1, 2, and 3 were incubated with CD19′ Raji cells and CD19⁻ 293 cells. Binding of CD19 Binders to cels was measured by flow cytometry. CD19 Binder 1 bound to CD19+ Raji cells and did not bind CD19⁻ 293 cells (FIGS. 4A, 4B). CD19 Binder 1 exhibited an EC₅₀ of 0.041 μg/mL (FIG. 4C). CD19 Binder 2 bound to CD19′ Raji cells and did not bind CD19⁻ 293 cells (FIGS. 5A, 5B). CD19 Binder 2 exhibited an EC₅₀ of 0.073 pg/mL (FIG. 5C). CD19 Binder 3 bound to CD19′ Raji cells and did not bind CD19-293 cells (FIGS. 6A, 6B). CD19 Binder 3 exhibited an EC₅₀ of 1.2 μg/mL (FIG. 6C).

### Example 3: In vivo characterization of CD19 CAR-T Cells

This example describes methods used to characterize the efficacy of CD19 CAR-T cell constructs in a B-cell tumor animal model. Table 33 provides an overview of the different experimental groups for the in vivo study.

In vivo characterization of CD19 CAR efficacy was performed as follows: Seven days prior to injection with CAR-T cells, 6-12 week old NOD.Cg-Prkdc^{scid}ll2rg^{tm1Wji1}/SzJ (NSG) mice (The Jackson Laboratory) were intravenously injected with Nalm6:Wasabi-ffLuc cells (5×10⁵ cells/mouse) according to the experimental groups outlined in Table 33. Live imaging was performed one day later. Four days after injection of the NALM-6 tumor cells, mice were intravenously injected with CAR-T cells (4×10⁶ cells/mouse) according to the experimental groups outlined in Table 33. Four days after injection of the CAR-T cells, in vivo bioluminescent imaging was performed, and was repeated at day 7, 11, 14, 18, 21, 25, 28, and 34 post-injection of CAR-T cells). In vivo five imaging was performed at the indicated timepoints using an IVIS in vivo imaging instrument (Perkin-Elmer). In vivo live imaging measured bioluminescence via intraperitoneal injection of D-luciferin substrate (Perkin-Elmer) All images were analyzed using Living Image software (Perkin-Elmer). Survival of CAR-T treated mice was measured and reported using Kaplan-Meier curves. Area under curves for treatment groups were compared using one-way ANOVA. All in vivo animal studies were conducted in compliance with Institutional Animal Care and Use Committee (IACUC) approved protocols.

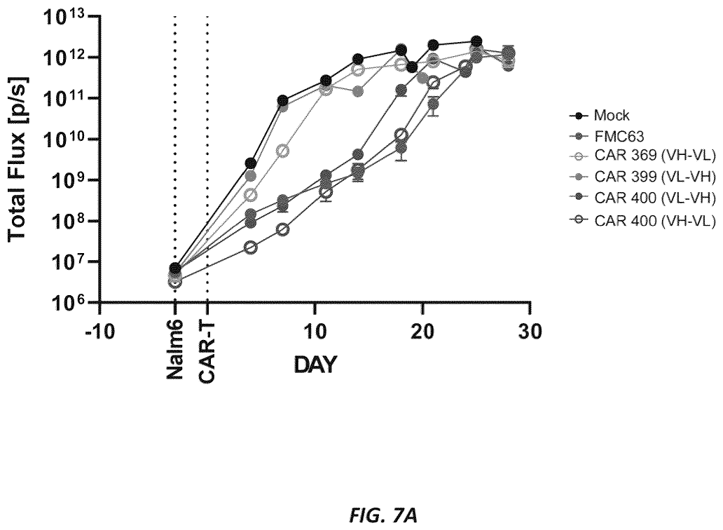

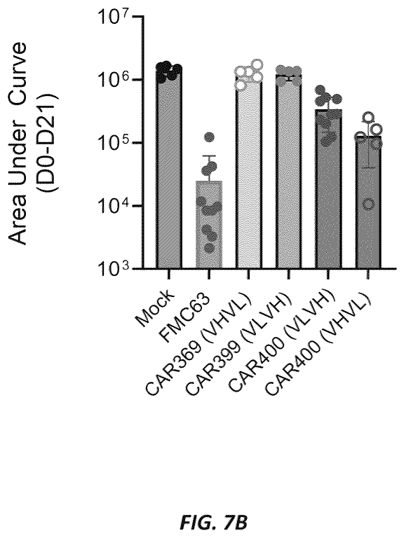

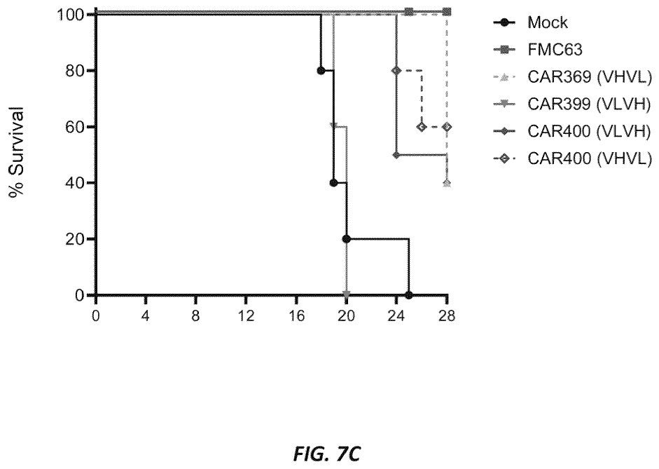

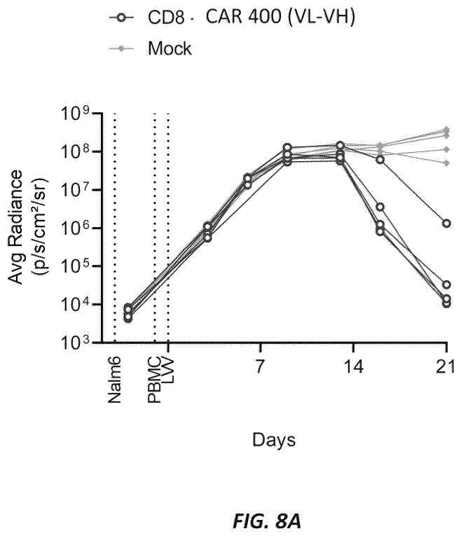

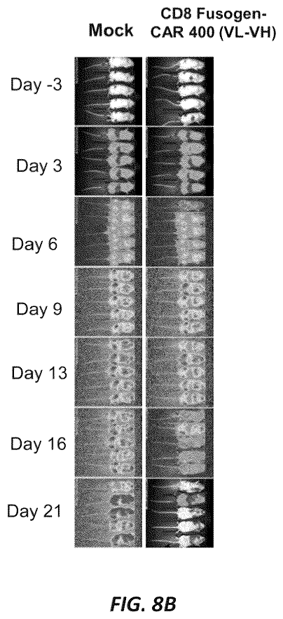

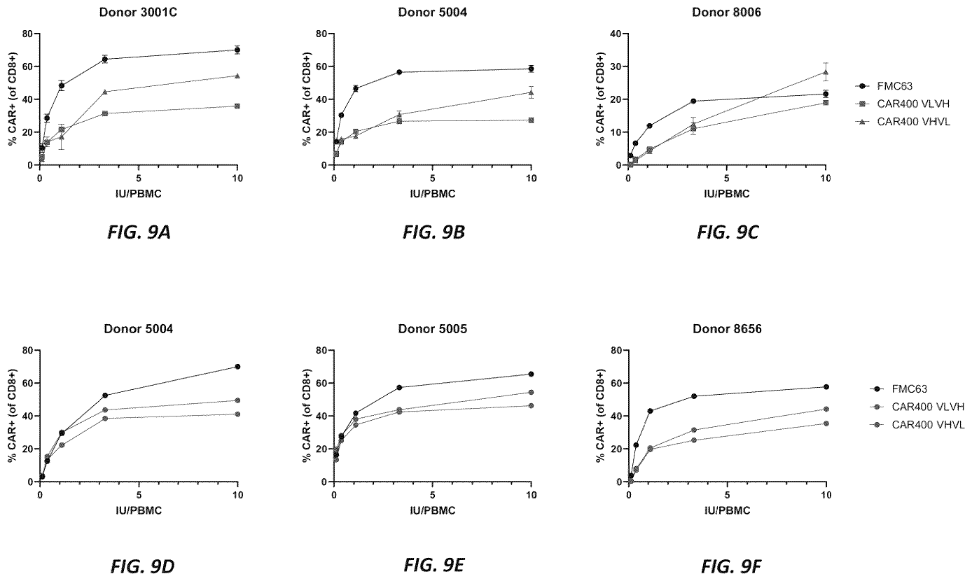

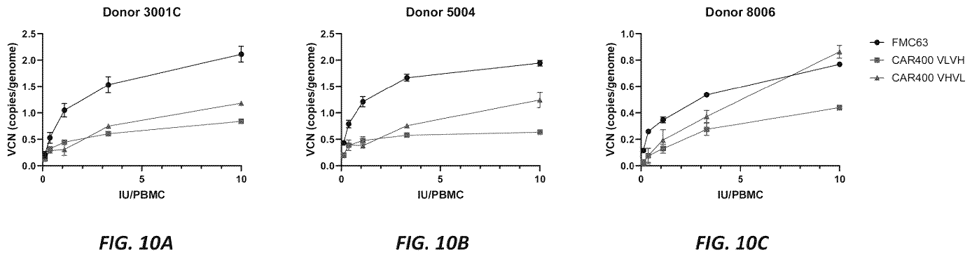

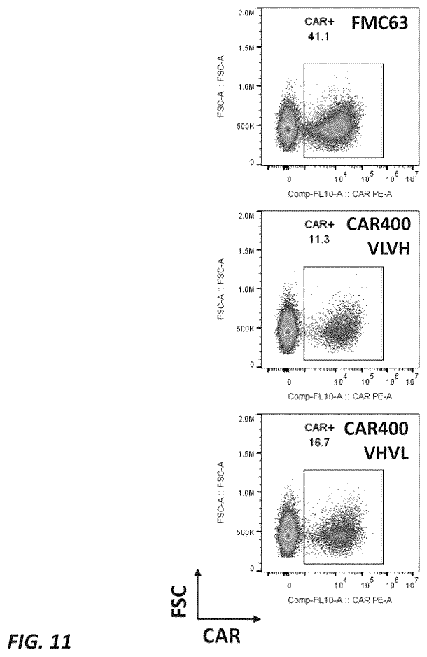

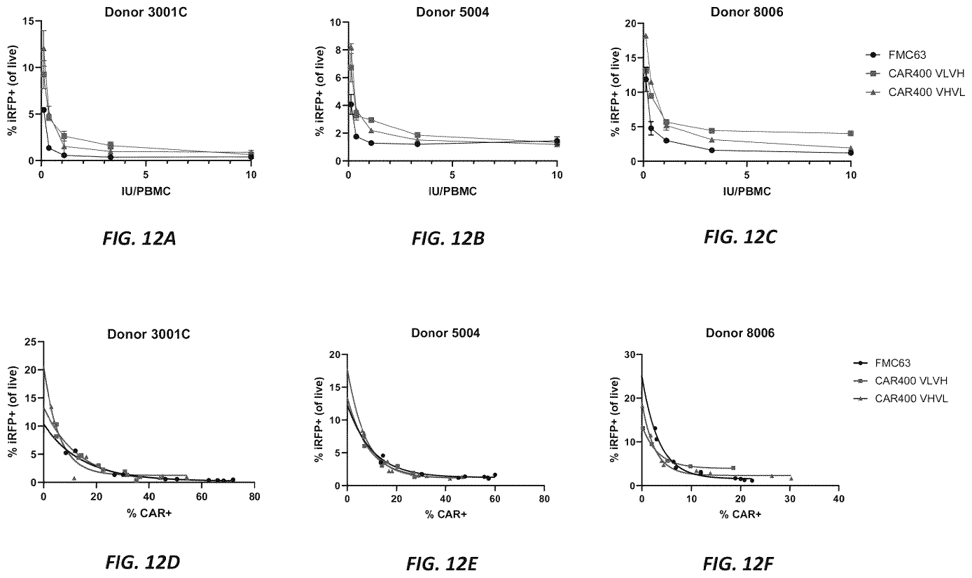

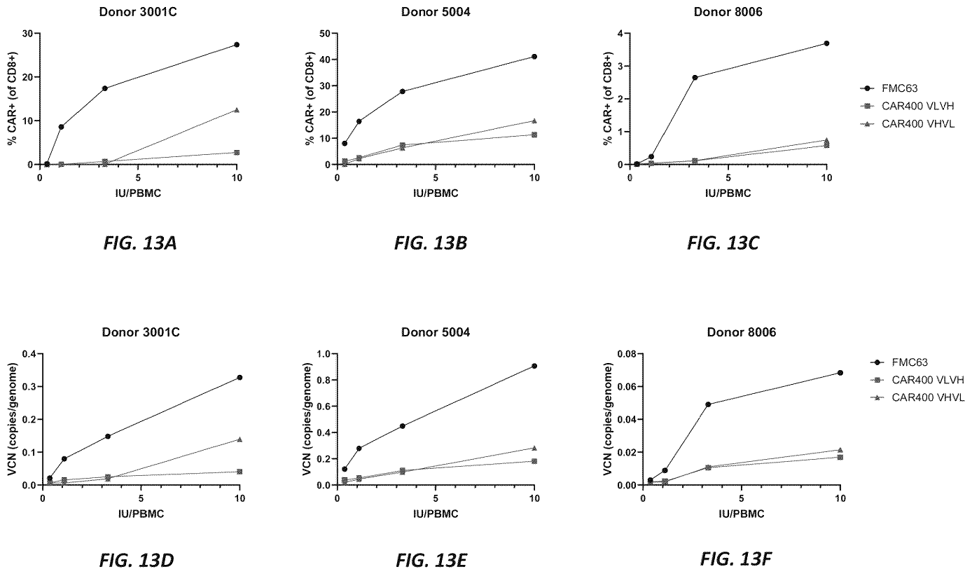

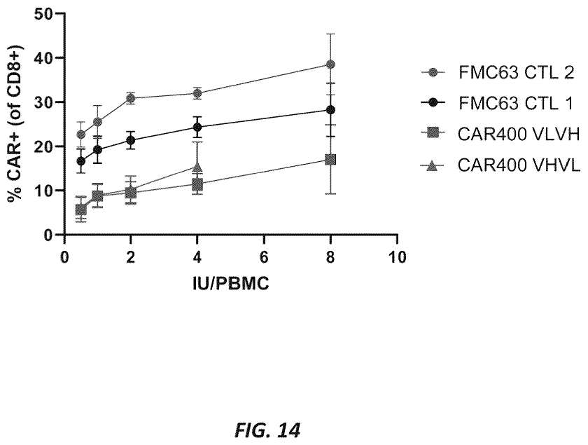

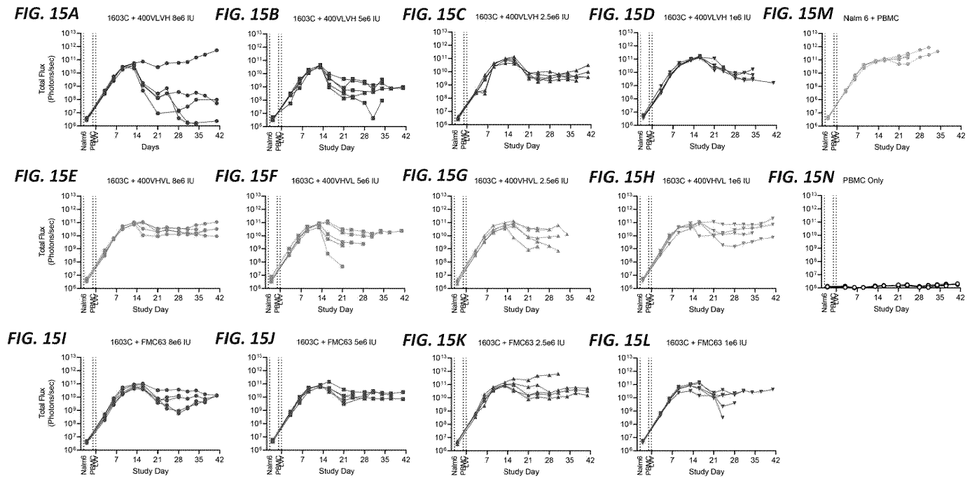

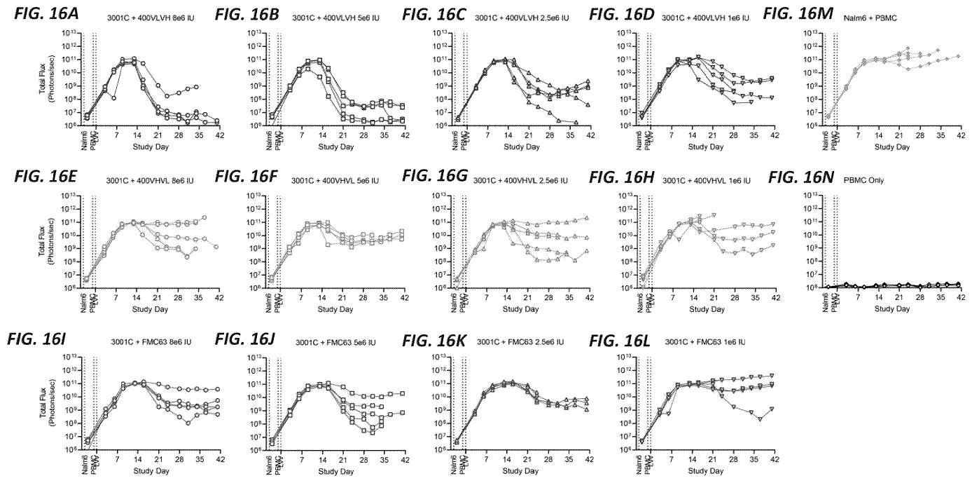

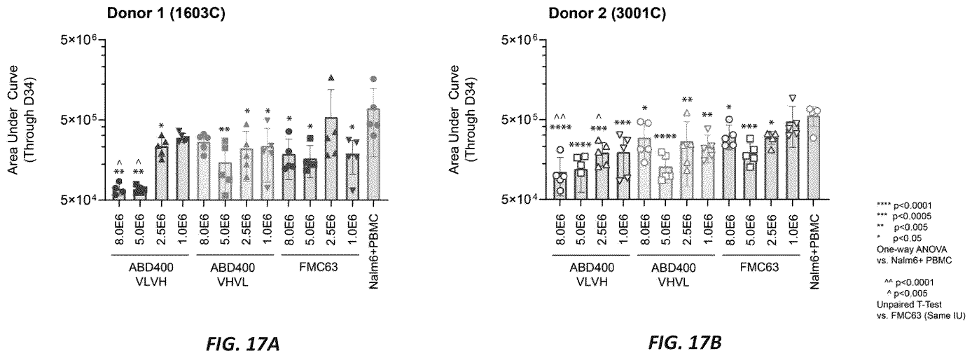

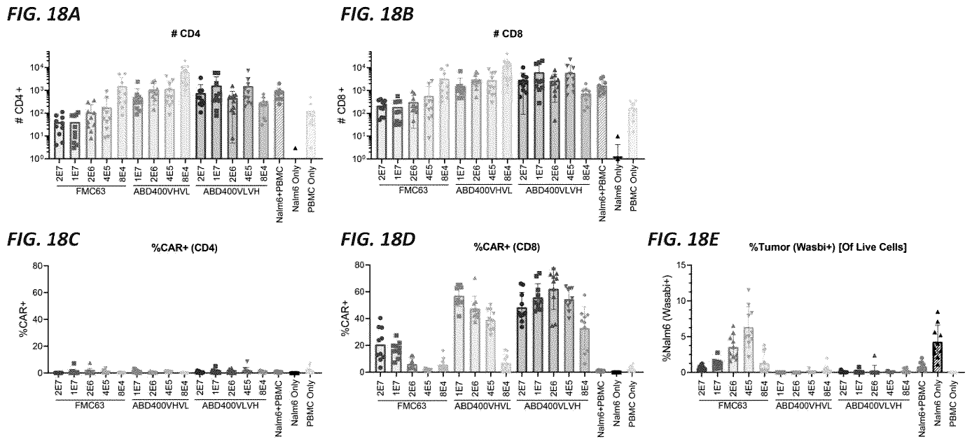

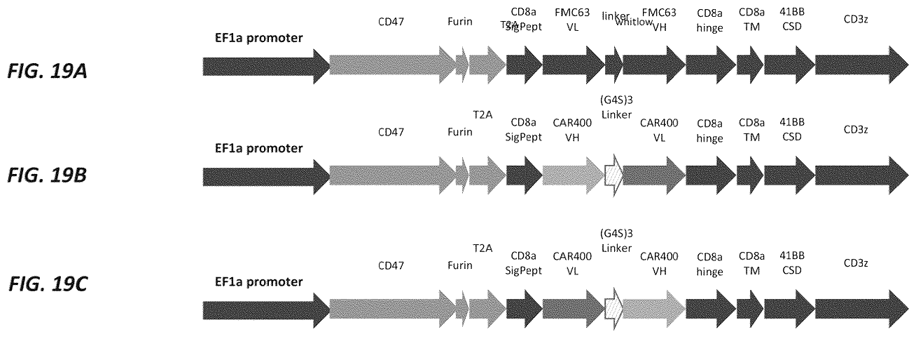

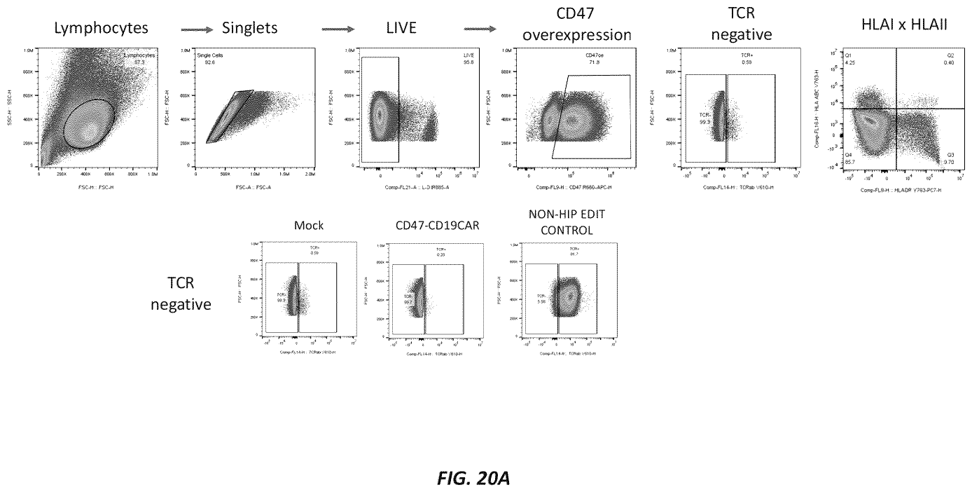

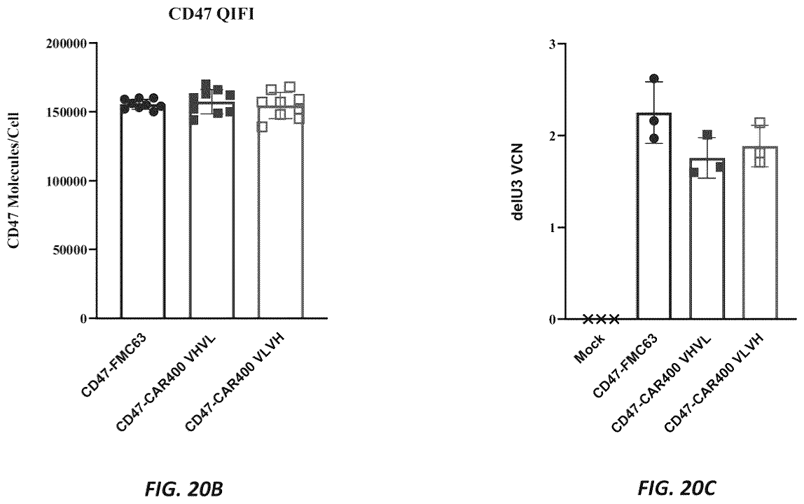

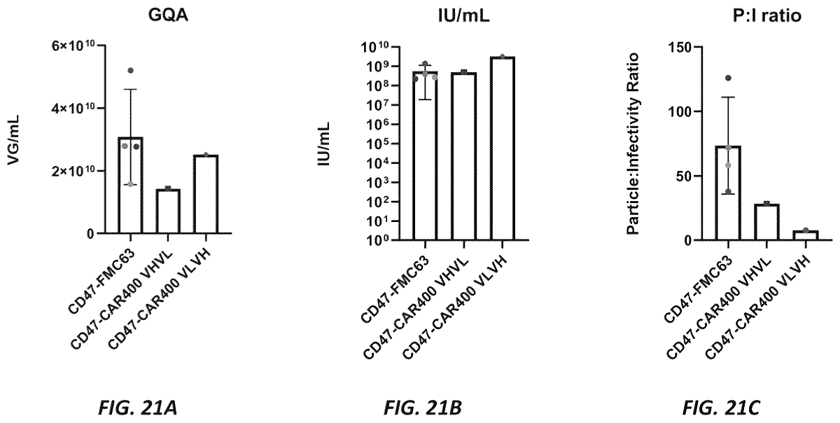

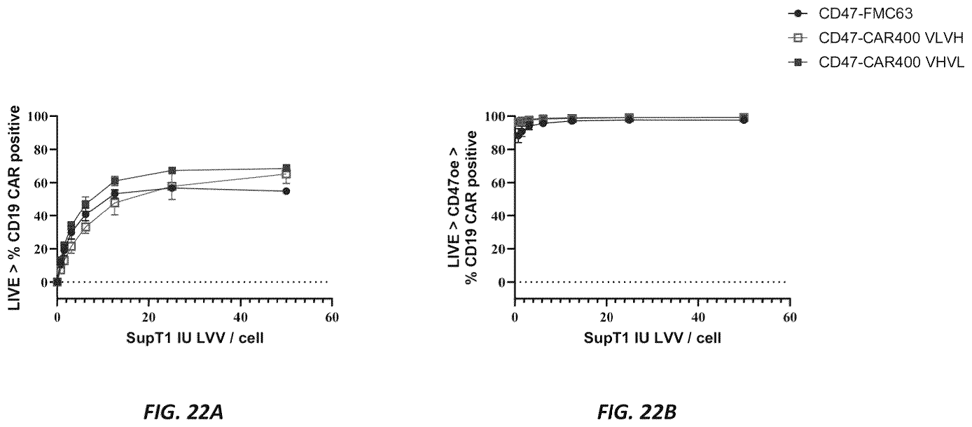

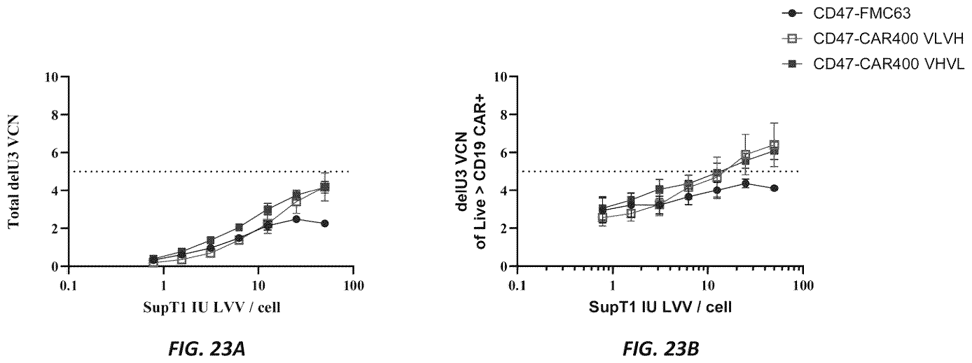

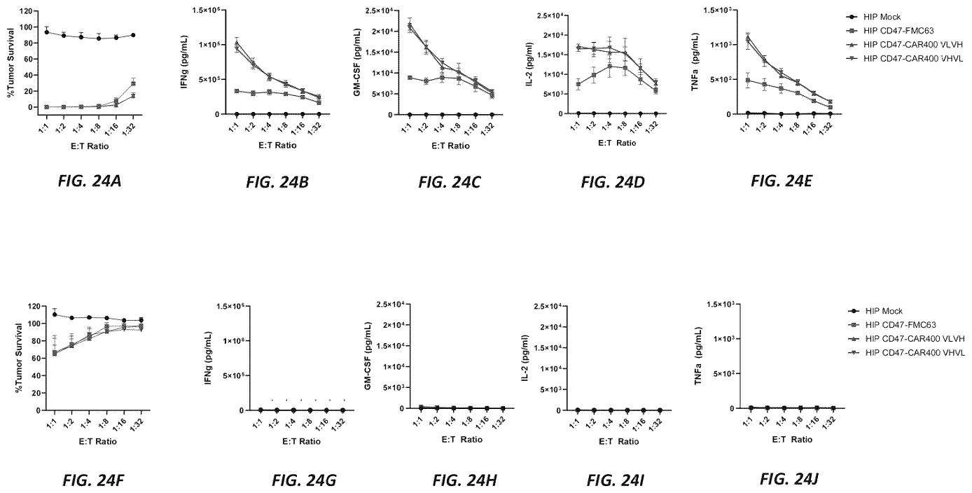

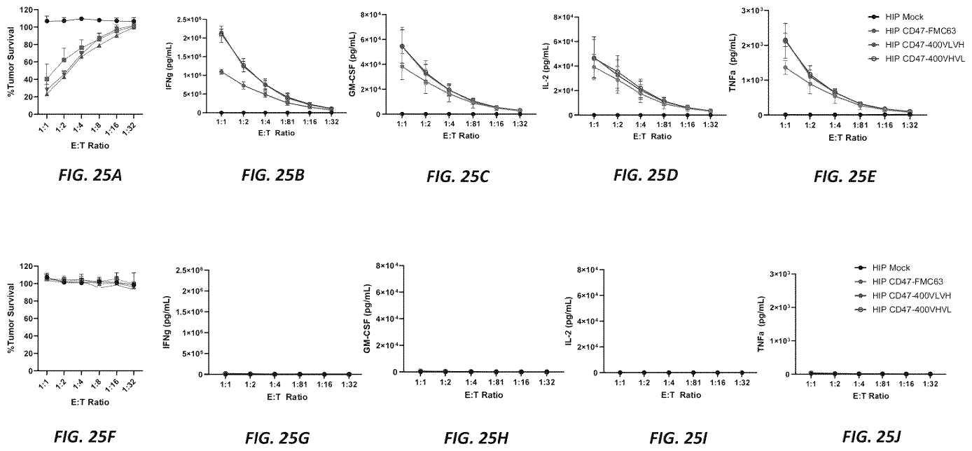

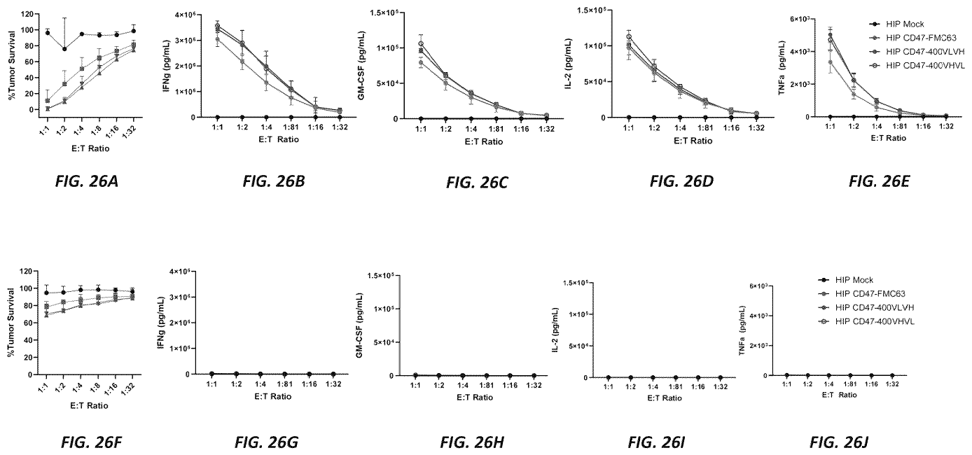

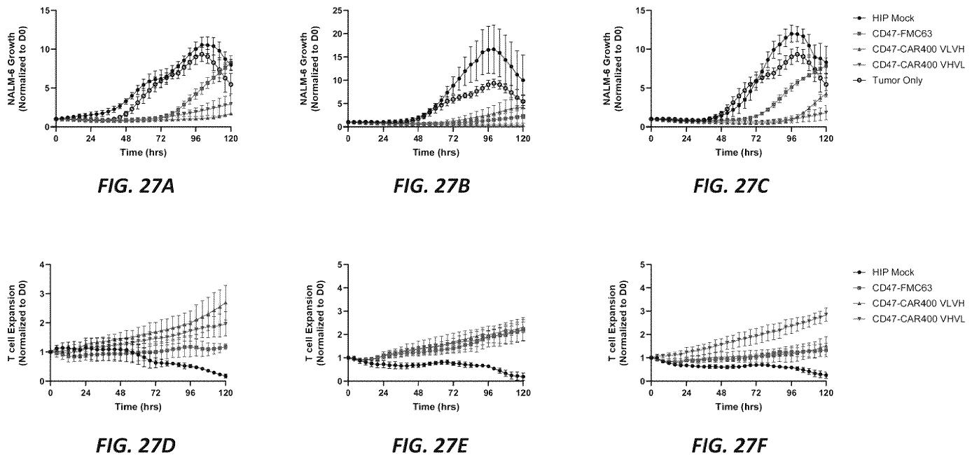

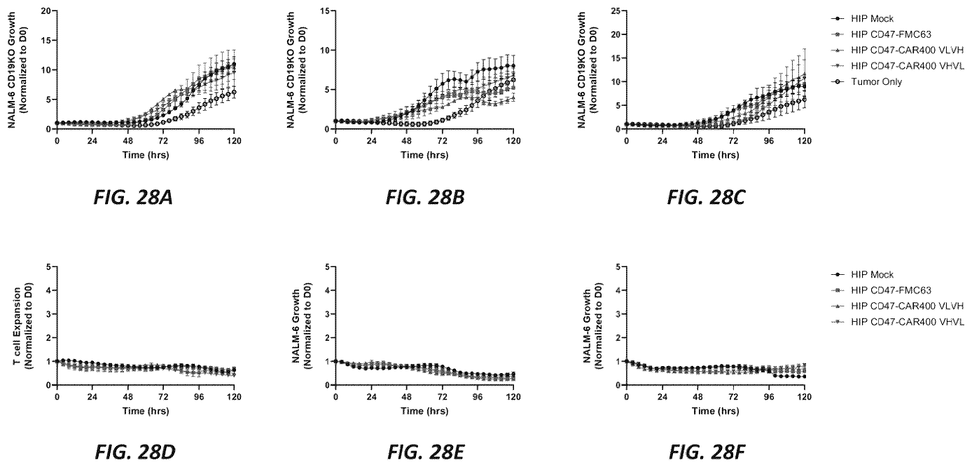

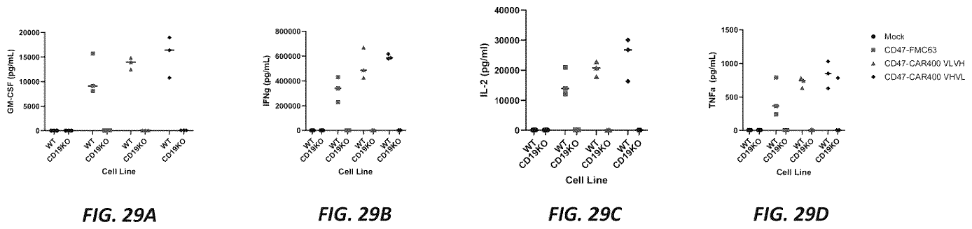

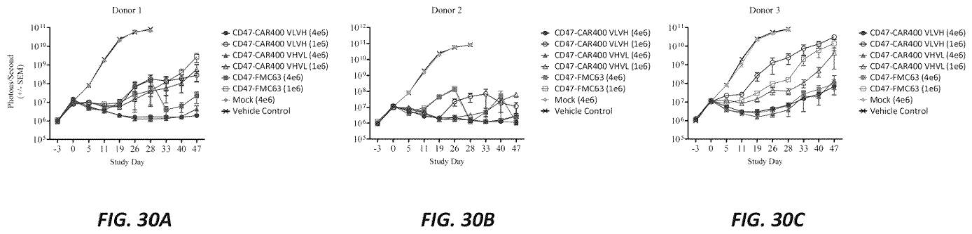

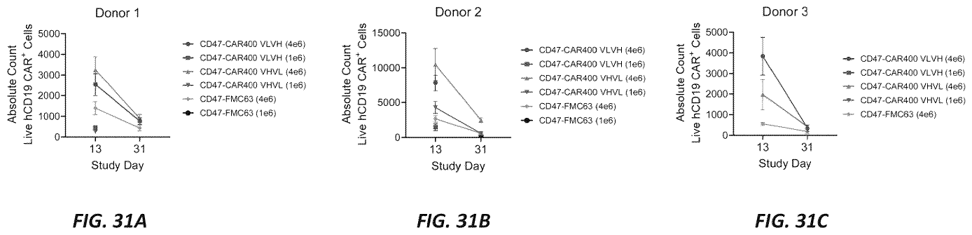

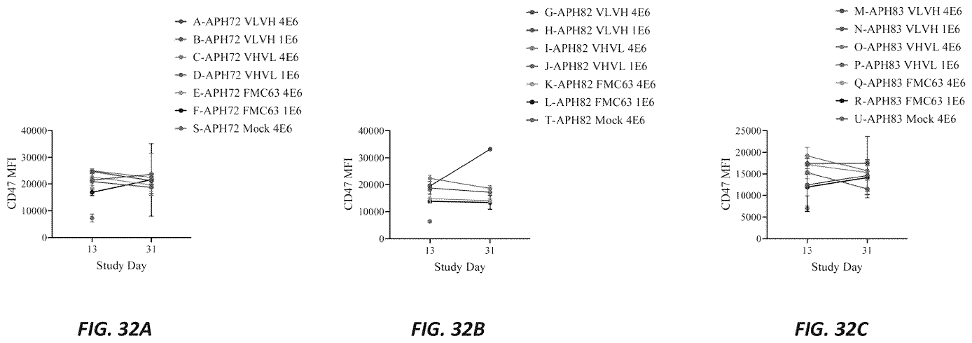

FIG. 7A depicts total flux (p/s) that illustrates tumor growth over a 28-day period after mice were injected with CD19 CAR-T cells. CAR 369 (VH-VL) and CAR 399 (VL-VH) exhibited tumor control that was similar to animals that received the tumor cell injection and mock CAR-T cells. CAR 400 (VL-VH) exhibited tumor cell control that was similar to the FMC63 benchmark C019 CAR over the first 14 days, and then exhibits tumor control that was less effective than the FMC63 benchmark CAR. CAR 400 (VL-VH) demonstrated improved tumor cell control relative to mock and CAR 369 (VH-VL) and CAR 399 (VL-VH). CAR 400 (VH-VL) demonstrated improved tumor cell control as measured by flux over the first 11 days of the study, and then demonstrated tumor cell control that was similar to the FMC63 benchmark over the remaining days of the study. CAR 400 (VH-VL) demonstrated the best in vivo tumor control of all tested CD19 CARs.

FIG. 7B shows the area under the curve (AUC) for all treatment groups. Animals receiving CAR 369 (VH-VL) and CAR 399 (VL-VH) exhibited an AUC similar to the mock treatment group. Animals receiving CAR 400 (VL-VH) exhibited a lower AUC relative to mock and CAR 369 (VH-VL) and CAR 399 (VL-VH). Animals receiving CAR 400 (VH-VL) exhibited an AUC that was not significantly different from the AUC for animals receiving the FMC63 benchmark CD19 CAR. Animals receiving the FMC63 benchmark CD19 CAR exhibited an AUC that was significantly lower (p<0.01) than all other treatment groups except for animals receiving CAR 400 (VH-VL) (p=0.8414).

FIG. 7C illustrates the survival of mice after injection with NALM-6 tumor cells followed by administration of CD19 CAR-T cells. Animals receiving CAR 399 (VL-VH) exhibited survival that was similar to animals receiving mock transduced cells. CAR 400 (VL-VH) exhibited improved survival, with 50% of animals surviving through day 24, and 40% of animals surviving through the 28-day study period. Animals receiving CAR 369 (VH-VL) also exhibited improved survival, with no animal succumbing to disease prior to day 28, when 60% of animals succumbed to disease, resulting in a 40% survival through the 28-day study period. Animals receiving CAR 400 (VH-VL) exhibited survivals closest to the FMC63 benchmark CD19 CAR. Eighty percent (80%) of animals receiving CAR 400 (VH-VL) survived through day 24, and 60% of animals receiving CAR 400 (VH-VL) survived through the 28-day study period, whereas 100% of animals receiving the FMC63 benchmark CD19 CAR survived.

### Example 4: In Vivo Characterization of Fully Humanized CD19 CARs

This example describes methods used to characterize the efficacy of fully humanized CD19 CAR constructs in a B-cell tumor animal model. Table 34 provides an overview of the different experimental groups for the in vivo study.

In vivo characterization of CD19 CAR efficacy was performed as follows: Four days prior to injection with CAR constructs, 6-12 week old NOD.Cg-Prkdc^{scid}ll2rg^{tm1Wj1}/SzJ (NSG) mice (The Jackson Laboratory) were intravenously injected with Nalm6:Wasabi-ffLuc cells (2.5×10⁵ cells/mouse) according to the experimental groups outlined in Table 34. Live imaging was performed one day later. One day prior to injection of the CD19 CAR constructs mice were intravenously injected with 1.0×10⁷ peripheral blood mononuclear cells (PBMCs). Four days after injection of the Nalm6 tumor cells and one day after injection of the PBMCs, mice were is intravenously injected with a CD8-FMC63 fusosome, a CD8-CAR 400 (VL-VH) fusosome (8×10⁶ IU/mouse, 5×10⁶ IU/mouse, 2.5×10⁶ IU/mouse, 1×10⁶ IU/mouse), or saline (mock), according to the experimental groups outlined in Table 34. In vivo bioluminescent imaging was performed on day 3, 6, 9, 13, 16, 21, 24, 28, 31, 34, 37, 41, and 44 following injection of the CD8− CD19 CAR fusosome. In viv live imaging was performed at the indicated timepoints using an IVIS In vivo imaging instrument (Perkin-Elmer). In vivo live imaging measured bioluminescence via intraperitoneal injection of D-luciferin substrate (Perkin-Elmer) All images were analyzed using Living Image software (Perkin-Elmer). Survival of CAR-T treated mice was measured and reported using Kaplan-Meier curves. All 0 vivo animal studies were conducted in compliance with Institutional Animal Care and Use Committee (IACUC) approved protocols.

FIG. 8A depicts average tumor radiance over the course of the study. Starting around day 14, animals receiving injection of the CD8-CAR 400 (VL-VH) fusosome exhibited a marked decrease in average tumor volume relative to animals receiving mock treatment. At day 21, several animals receiving the CDB-CAR 400 (VL-VH) fusosome exhibited an average radiance of approximately 104 whereas animals receiving the mock treatment exhibited an average radiance of approximately 10⁸. FIG. 8B shows tumor radiance in mock treated animals and animals receiving the CD8-CAR 400 (VL-VH) fusosome. While animals receiving the CD8-CAR 400 (VL-VH) fusosome exhibited tumor radiance similar to mock treated animals over the first 13 days, animals receiving the CD8-CAR 400 (VL-VH) fusosome exhibited reduced tumor radiance relative to mock treated animals on days 16 and 21.

### Example 5: Evaluation of CD8-Retargeted Fusogens Having an Anti-hCD19 CAR

This example describes the methods used to evaluate CD8-retargeted fusogens having an anti-hCD19 CAR (i.e., CAR400 VHVL and CAR400 VLVH) in vitro and in vivo, and the results of these evaluations.

### Integration in Transduced Cells

Digital droplet PCR (ddPCR) was used to determine the average integrated copy number of lentiviral genomes in a transduced cell population. Following transduction of cells as previously described, cells in a multi-well plate were pelleted, supernatant was removed, and cells were lysed by the addition of 200 μL of lysis buffer (20 mL warmed Lysis Buffer T1 combined with 2.6 mL of Proteinase K solution (Proteinase K with 2.6 mL of Proteinase K Buffer PB); Macherey-Nagel). Wells were mixed thoroughly with a P1000 pipette to ensure that cell pellets were homogenized in the lysis buffer. The plate was then incubated at 56° C. for 10 minutes. A 1:1 mixture of Buffer BQ1 (22 mL; Macherey-Nagel) and 96-100% ethanol (22 mL) was prepared, and 400 μL of the BQ1-ethanol mixture was added to each well and wells were mixed. 600 μL of the lysate was then transferred to a Nudeospin Tissue Binding Plate (Macherey-Nagel) and sealed with PE foil. The plate was then centrifuged at 4000 g for 16 minutes.

The silica membrane was then washed by centrifuge processing and/or vacuum processing. For centrifuge processing, 500 μL of Buffer BW (Macherey-Nagel) was then added to each well, the plate was sealed again and centrifuged at 4000 g for 4 minutes. After confirming that all buffer passed through the wells, a second wash of the wells was performed by adding 700 μL of Buffer B5 (Macherey-Nagel) to each well and the plate was again sealed and centrifuged at 4000 g for 4 minutes. For vacuum processing, 600 μL of Buffer BW (Macherey-Nagel) was added to each well and a vacuum was applied for 1-2 minutes until all buffer had passed through the wells. A second and third vacuum wash was performed by adding 900 μL of Buffer B5 (Macherey-Nagel) and a vacuum was applied for 1-2 minutes until all buffer had passed through the wells.

The silica membrane was then dried by centrifuge processing and/or vacuum processing. For centrifuge processing, the plates were incubated on a MN Square-well block for 20 minutes at 37° C. to evaporate any residual ethanol. For vacuum processing, the plate was placed on a vacuum for at least 20 minutes. For both methods, it was confirmed that the membrane was completely dry before moving on to the DNA elution step.

DNA was eluted by placing the Nucleospin plate on a semi-skirted plate, and 45-100 μL of Buffer BE (preheated to 56-70° C.; Macherey-Nagel) was added directly to the membrane of each well. The plate was then incubated for 1 minute at room temperature. DNA was then eluted by centrifuge processing and/or vacuum processing. For centrifuge processing, the plate was centrifuged at 3000 g for 2 minutes. For vacuum processing, a vacuum was applied to the plate for 1-2 minutes. Concentration of gDNA in the eluted sample was measured by nanodrop, and samples were diluted, if need, so that the concentration of gDNA was 10-50 ng/μL.

ddPCR was performed by adding 22 μL of master mix (volume per well: 12.5 μL 2× ddPCR Supermix for Probes, no dUTP, 1.25 μL 20× delU3 primer/probe, 1.25 μL 20×ARX primer/probe, 0.3 μL EcoR1 (20,000 U/mL), and 9.7 μL H₂O) into each well containing a sample for the assay. 3 μL of eluted gDNA or H₂O (control) was added to the wells and plate was sealed and centrifuged at 3000 g for 2 minutes.

An automated droplet generator was then used for droplet generation, ddPCR was then run on a C1000 Touch Thermal Cycler with a deep well block. The plate was then loaded onto a QX200 droplet reader and analyzed.

### Activated PBMC Transduction

The following experiment assessed the efficacy of fusogens carrying CARs with different CD19 binders (FMC63, CAR400 VHVL, and CAR400 VLVH) in a primary human PBMC in vitro model, and CAR expression and NALM-6 killing by CD8+ T cells were evaluated. CARs were introduced into a 3L production of lentiviral vectors pseudotyped with a CD8-retargeted fusogen. Primary human PBMCs from three donors were thawed and resuspended (40 million cells per donor in 20 mL OpTmizer medium with 100 units/ml IL-2). Five hours after the thaw, CTS beads were added at a 3:1 bead:cell ratio. The next day, PBMCs were plated (100,000 PBMCs/well) and then transduced with the CARs by spinfection (60 minutes at 1000g and 32° C.) using a three-fold dilution series ranging from 0.1 IU/PBMC to 10 IU/PBMC. Three days later, cells were de-beaded, and media was replaced with 200 μL fresh media containing 100 units/mL IL-2. 100 μL of cells were then used to assess NALM-6 cell killing, and the other 100 p1 was stained and assessed by flow cytometry.

To assess NALM-6 cell killing, 80,000 NALM6 iRFP were seeded into wells of a multi-well plate in 100 μL. 100 μL of transduced cells were then added to the NALM-6 cells. Cells were mixed and then left at room temperature for 30 minutes before being placed in an Incucyte imager where scans were taken every 4 hours (as described above).

For flow cytometric analysis, cells were stained with rec hCD19-Fc (or anti-FMC63), and CAR expression was then assessed by flow cytometry.

These experiments were additionally replicated using three additional donor cell lines.

FIGS. 9A-9F show PBMC CAR expression following transduction with spinfection in activated PBMCs. FIGS. 9A-9C and 9D-9F represent results from repeats of the study using various donor cell lines to evaluate CAR expression. Generally, the two hCD19 CAR constructs (CAR400 VHVL and CAR400 VLVH) exhibited similar expression in activated PBMCs, and the expression level (i.e., transduction rate) was slightly lower than the FMC63 CAR as the viral vector concentration increased. The different donor lines generally exhibited similar transduction rates, with the PBMCs from Donor 8656 (FIG. 9F) showing slightly lower CAR expression (i.e., transduction rate) relative to the other donor PBMCs.

FIGS. 10A-10C show the vector copy number (VCN) per genome, which shows the number of viral vector genomes that are integrated into the genome of the target cells. Similar to the CAR expression results, CAR400 VHVL and CAR400 VLVH exhibited similar VCNs, and these were at a level that was slightly lower than that of FMC63. Importantly, there was consistency between CAR expression and integration (VCN) in the transduced cells.

FIG. 11 depicts representative staining from flow cytometry analyses measuring the CAR expression in transduced cells. As can be seen, FMC63 showed the highest level of CAR expression, followed by CAR400 VHVL, and then CAR400 VLVH.

FIGS. 12A-12C show the functional activity of the CARs in killing NALM-6 cells. All three tested CARs were effective in killing NALM-6 tumor cells when the dose of CARs was approximately 1 IU/PBMC and higher. FIGS. 12D-12F show the functional activity when normalized to CAR+ cells, allowing for comparison at similar ranges of E:T ratios. Further, when normalized to CAR+ cells, functional activity of CAR400 VHVL and CAR400 VLVH was similar to FMC63, and the CARs demonstrated effective killing of NALM-6 tumor cells (FIGS. 12D-12F).

### ECD-Like State Transduction

The following experiment assessed the efficacy of fusogens carrying CARs with different CD19 binders (FMC63, CAR400 VHVL, and CAR400 VLVH) in an extracorporeal dosing (ECD)-like context (e.g., resting PBMCs) in which fusogens and cells are incubated at a high concentration for 2 hours. Primary human PBMCs from 3 donors were thawed and resuspended (14 million cells in 140 μL). 10 μL of resuspended cells (1×10⁶ live PBMCs) was added into each well of a 96-well U-bottom tissue culture-treated plate and incubated at 37° C. Lentiviral vectors were prepared at a 1:3 dilution series (10-0.37 IU/cell final), added to the appropriate wells and incubated at 22° C. for 2 hours. Cells were then washed by centrifuging at 750 g for 3 minutes, washed again with 200 μL PBS, and then resuspended in 200 μL PBS. 100 μL of transduced cells were reserved for staining and analysis by flow cytometry, and the remaining 100 μL were reserved for activation. For activation, the plate was centrifuged and cells were resuspended in 200 μL RPMI+10% FBS with 100 units/mL IL-2 and 7.5×10⁵CTS Dynabeads (3:1 PBMC:bead ratio). Cells were then incubated. After 3 days, cells were de-beaded, most wells were split 1:2, and wells were replenished with 200 μL fresh media. Two days later, cels were again split 1:2.

To assess NALM-6 killing, 15,000 NALM-6 iRFP713 cells were added per well in a multi-well plate. Two-fold serial dilutions of transduced cells (E:T ratio ranging from 1:1 to 1:16) were added, cells were mixed, and then left at room temperature for 30 minutes before placing in an Incucyte imager where scans were taken every 4 hours (as described above). Three days after the start of the assessment of NALM-6 killing, cells were transferred to a new plate, and half of the media was replaced with fresh media (no IL-2 in the media) containing 15,000 additional NALM-6 iRFP713 cells/well (restimulation). After an additional three days, the cells were again transferred to a new plate and half of the media was replaced with fresh media (no IL-2 in the media) containing 15,000 additional NALM-6 iRFP713 cells/well (restimulation).

For flow cytometric analysis, cells were stained with rec hCD19-Fc (or anti-FMC63), and CAR expression was then assessed by flow cytometry.

FIGS. 13A-13C show CAR expression following transduction of resting PBMCs in an ECD-like setting. Generally, the two hCD19 CAR constructs (CAR400 VHVL and CAR400 VLVH) exhibited similar expression in transduced resting PBMCs, and the expression level (i.e., transduction rate) was lower than the FMC83 CAR, and this observation continued as the viral vector concentration increased. Transduction rates in resting PBMCs were also lower than that of activated PBMCs. FIGS. 13D-13F show the vector copy number (VCN) per genome, which shows the number of viral vector genomes that were integrated into the genome of the target cells. Similar to the CAR expression results, CAR400 VHVL and CAR400 VLVH exhibited similar VCNs, and these were at a level that was lower than that of FMC63. Importantly, there was consistency between CAR expression and integration (VCN)

A repeat of the ECD-like state transduction experiment, with the inclusion of SBLV22015 as an additional positive control, demonstrated higher expression of CAR400 VHVL and CAR400 VLVH in resting PBMCs, but this expression was still less than FMC63 and SBLV22015 (FIG. 14). It was noted that expression of FMC63 and SBLV22015 was confirmed by both hCD19-Fc and anti-FMC63 antibodies, and that the expression of CAR400 VHVL was not tested at a vector concentration of 8 IU/PBMC due to insufficient material for the testing.

In Vivo Assessment of CD64Targeted Fusogens Having hCD19 CARs

This example describes methods used to characterize and efficacy results of CD8-targeted fusogens having human CD19 CAR constructs (i.e., CAR400 VLVH and CAR400 VHVL) in a B-cell tumor animal model. Table 34 provides an overview of the different experimental groups for the in vivo study.

In vivo characterization of CD19 CAR efficacy was performed as follows: Four days prior to injection with CAR constructs, 6-12 week old NOD.Cg-Prkdc^{scid}ll2rg^{tm1Wjl}/SzJ (NSG) mice (The Jackson Laboratory) were intravenously injected with Nalm6:Wasabi-ffLuc cells (2.5×10⁵ cells/mouse) according to the experimental groups outlined in Table 34. Live imaging was performed one day later. One day prior to injection of the CD19 CAR constructs mice were intravenously injected with 1.0×107 PBMCs. Four days after injection of the Nalm6 tumor cells and one day after injection of the PBMCs, mice were intravenously injected with a CD8-FMC63 fusogen (8×10⁶ IU/mouse, 5×10a IU/mouse, 2.5×10⁶ IU/mouse, 1×10V IU/mouse), a CD8-CAR400 (VL-VH) fusogen (8×106 IU/mouse, 5×10 ILU/mouse, 2.5×10⁸ IU/mouse, 1×10⁶ IU/mouse), a CD8-CAR400 (VH-VL) fusosome (8×10⁶ IU/mouse, 5×100 IU/mouse, 2.5×10⁶ IU/mouse. 1×106 IU/mouse),or saline (mock), according to the experimental groups outlined in Table 34. In vivo bioluminescent imaging was performed on day 3, 6, 9, 13, 16, 21, 24, 28, 31, 34, 37, 41, and 44 following injection of the fusogens. In vivo live imaging was performed at the indicated timepoints using an IVIS in vivo imaging instrument (Perkin-Elmer). In vivo live imaging measured bioluminescence via intraperitoneal injection of D-luciferin substrate (Perkin-Elmer) All images were analyzed using Living Image software (Perkin-Elmer). Survival of CAR-T treated mice was measured and reported using Kaplan-Meier curves. All in vivo animal studies were conducted in compliance with Institutional Animal Care and Use Committee (IACUC) approved protocols.

The efficacy of CD8-CD19CAR fusogens was assessed in the animal model described above. FIGS. 16A-15D show the total flux for animals receiving tumor cells and CAR400 VLVH (at doses of 8×10⁶ IU, 5×10⁶ IU, 2.5×10⁶ IU, and 1×10⁶ IU, respectively). FIGS. 16E-15H show the total flux for animals receiving tumor cells and CAR400 VHVL (at doses of 8×108 IU, 5×10⁰ IU, 2.5×10⁶ IU, and 1×10⁶ IU, respectively). FIGS. 161-15L show the total flux for animals receiving tumor cells and FMC63 (at doses of 8×10⁶ IU, 5×10⁶ IU, 2.5×10⁶ lU, and 1×10⁶ IU, respectively). FIG. 15M shows flux in animals receiving tumor cells and untransduced PBMCs that do not express a CAR, and FIG. 16N shows flux in animals receiving PBMCs alone (i.e., no tumor cells).

The efficacy of CD8-CD19CAR fusogens was also assessed in the animal model described above, using PBMCs from a second donor (3001C). FIGS. 16A-16D show the total flux for animals receiving tumor cells and CAR400 VLVH (at doses of 8×10⁶ IU, 5×10⁶ IU, 2.5×10⁶ IU, and 1×10 IU, respectively). FIGS. 16E-16H show the total flux for animals receiving tumor cells and CAR400 VHVL (at doses of 8×10⁶ IU, 5×100⁶ IU, 2.5×10⁶ IU, and 1×10⁸ IU, respectively). FIGS. 161-16L show the total flux for animals receiving tumor cells and FMC63 (at doses of 8×10⁶ IU, 5×10⁶ IU, 2.5×100⁶ IU, and 1×10⁶ IU, respectively). FIG. 16M shows flux in animals receiving tumor cells and untransduced PBMCs that do not express a CAR, and FIG. 16N shows flux in animals receiving PBMCs alone (i.e., no tumor cells).

Consistent across both PBMC donors, CAR400 VLVH demonstrated dose-dependent reductions in total flux relative to the control group (tumor cells +no CAR), with reduced flux relative to controls measured at all tested doses. CAR400 VHVL also showed reduced total flux relative to the control group (tumor cells +no CAR), albeit at a slightly lower level than CAR400 VLVH. The reductions in total flux for CAR400 VLVH and CAR400 VHVL were comparable to, or greater than the reduction in total flux in animals receiving FMC63 at all tested doses, and for both PBMC donors.

FIGS. 17A and 176 show the area under the curve (AUC) for the tumor burden through 34 days of the study for PBMCs from two different donors. Cut off date for calculation of AUC was set at first date of mortality for control groups. Notably, both CAR400 VLVH and CAR400 VHVL performed better than or similar to FMC63 across all tested doses and both PBMC donors. Both CAR400 VLVH and CAR400 VHVL demonstrated statistically significant reductions in the total tumor burden AUC relative the control group where animals did not receive a CAR. Additionally, statistically significant reductions in tumor burden were observed at doses of 2.5×10⁸ IU/mouse, 5×10° IU/mouse, and 8×10⁶ IU/mouse for CAR400 VLVH (as compared to FMC63 at the same dose).

A separate study using similar controls but with different doses is shown in FIGS. 18A-18B. For example, FIGS. 18A and 188 show numbers of CD4+ and CD8+ cells, respectively, detected in peripheral blood of animals in the study at day 14, as assessed by flow cytometry. The cell counts represent the number of cells per 100 μL blood. FIGS. 18C and 18D show the percentage of CAR positive cells in CD4+(FIG. 18C) and CD8+(FIG. 18D) cells. CD8+ CAR-T cells were detected in the peripheral blood of animals in a dose-dependent manner. FIG. 18E shows the percentage of tumor cells detected in peripheral blood as a percentage of total live cells. While animals in the FMC63 groups showed levels of tumor cells in peripheral blood that were similar to the control (tumor cells +no CAR), animals receiving CAR400 VHVL and CAR400 VLVH, had little, if any, tumor cells present in peripheral blood.

### Example 6: Evaluation of Hypoimmune T Cells Comprising an Anti-hCD19 CAR

This examples describes the methods used to evaluate hypoimmune CD47-CD19 CAR T cells comprising a hCD19 binder (i.e., CAR400 VLVH and CAR400 VHVL) and the tolerogenic factor CD47 (SEQ ID NO: 167), in vitro and in vivo, and the results of these evaluations.

### Integration in Transduced Cells

Digital droplet PCR (ddPCR) was used to determine the average integrated copy number of lentiviral genomes in a transduced cell population. Following transduction of cells as previously described, cells in a multi-well plate were pelleted, supernatant was removed, and cells were lysed by the addition of 200 μL of lysis buffer (20 mL warmed Lysis Buffer T1 combined with 2.6 mL of Proteinase K solution (Proteinase K with 2.6 mL of Proteinase K Buffer PB); Macherey-Nagel). Wells were mixed thoroughly with a P1000 pipette to ensure that cell pellets were homogenized in the lysis buffer. The plate was then Incubated at 56° C. for 10 minutes. A 1:1 mixture of Buffer BQ1 (22 mL; Macherey-Nagel) and 96-100% ethanol (22 mL) was prepared, and 400 μL of the BQ1-ethanol mixture was added to each well and wells were mixed. 600 μL of the lysate was then transferred to a Nucleospin Tissue Binding Plate (Macherey-Nagel) and sealed with PE foil. The plate was then centrifuged at 4000 g for 16 minutes.

The silica membrane was then washed by centrifuge processing. For the first wash, 500 μL of Buffer BW (Macherey-Nagel) was added to each well, the plate was sealed and centrifuged at 4000 g for 4 minutes. After confirming that all buffer passed through the wells, a second wash of the wells was performed by adding 700 μL of Buffer 85 (Macherey-Nagel) to each well and the plate was sealed and centrifuged at 4000 g for 4 minutes. The silica membrane was then dried by incubating the plate for 20 minutes at 37° C. to evaporate any residual ethanol.

DNA was eluted by placing the Nucleospin plate on a semi-skirted plate, and 100 μL of Buffer BE (preheated to 56-70° C.; Macherey-Nagel) was added directly to the membrane of each well. The plate was then incubated for 1 minute at room temperature. DNA was then eluted by centrifuging the plate at 3000 g for 2 minutes. Concentration of gDNA in the eluted sample was measured by nanodrop, and samples were diluted, if need, so that the concentration of gDNA was 10-50 ng/μL.

ddPCR was performed by adding 22 μL of master mix (volume per well: 12.5 μL 2× ddPCR Supermix for Probes, no dUTP, 1.25 μL 20× delU3 primer/probe, 1.25 μL 20× uTERT primer/probe, 0.3 μL EcoR1 (20,000 U/mL), and 9.7 μL H₂O) Into each well containing a sample for the assay. 3 μL of eluted gDNA or H₂O (control) was added to the wells and plate was sealed and centrifuged at 3000 g for 2 minutes.

An automated droplet generator was then used for droplet generation, ddPCR was then run on a C1000 Touch Thermal Cycler with a deep well block. The plate was then loaded onto a QX200 droplet reader and analyzed.

### Hypoimmune CAR T Cell Production

Hypoimmune CD47-CD19 CAR T cells having FMC63, CAR400 VLVH, or CAR400 VHVL, and that are TCR and HLA-I/II disrupted were prepared as follows.

On day 0, pan T cells were thawed in Complete CTS OpTmizer media (with 100 IU/mL IL-2) with CD4 and CD8 T cells thawed and cultured separately. That afternoon, T cells were stimulated with CTS Dynabeads at a 1:1 bead:cell ratio. The next morning (day 1), 5×10^{b} cells were added into 1 mL Complete CTS OpTmizer media (containing 100 IU/mL IL-2) in each well of a 12-well plate. Cells were then transduced by spinoculation (mock, CD47-FMC63 (FIG. 19A), CD47-CAR400 VHVL (FIG. 190), or CD47-CAR400 VLVH (FIG. 19C)) at 32° C. for 60 min at 1000×g. On day 2, 3 mL of CTS OpTmizer media (containing 100 IU/mL IL-2) was added to the transduction plates. On day 3, cells were removed from the wells and transferred to 50 mL conical tubes, and the remainder of the conical tube was filled with PBS. Cells were debeaded by placing the conical tube in a magnet for approximately 2 minutes and cells were then pipetted into a new 50 mL conical tube. This step (magnetic debeading and transfer of cells) was repeated as a secondary bead removal step to ensure all beads were removed. The cells were resuspended in 9 mL of CTS OpTmizer+IL-2, and 3 mL of cells was transferred to a 2-well G-Rex plate (non-hypoimmune edited cells). Cell counts were then performed on the debeaded cells.

For cell editing, a master mix was prepared (volume per 100 pt: 1.25 μL Cas12b mRNA (TriLink), 1.25 μL B2M gRNA (Synthego), 1.25 μL CIITA gRNA (Synthego), 1.25 μL TRAC gRNA (Synthego), and 80 μL P3 Buffer (Lonza). Debeaded cells (5×10⁶ cells) were harvested and pelleted at 90×g for 10 mins. The supernatant was carefully aspirated, and cells were resuspended in 85 μL of the master mix described above. The resuspended cells were immediately transferred to a 100 μL nucleocuvette and nucleofected using program DN-130 on a 4d Nucleofector (Lonza). Cells were recovered by slow addition of 400 μL of recovery media to the side of each well, followed by incubation at 37° C. for 10 min. Cells were then transferred from the nucleocuvette to a 24-well G-Rex plate. Three days later, cells were transferred to a 6-well G-Rex plate if confluent, and IL-2 was replenished. The following day, a subset of cells were analyzed by flow cytometry to measure marker expression, and cells were then frozen down two days later. FIG. 20A shows a representative flow cytometry gating strategy and staining from cells. Expression of CD47 in the target cells was confirmed by quantitative flow cytometry analysis (QIFI: FIG. 20B), and expression of CARs was confirmed by measurement of integration into target cells as measured by ddPCR (FIG. 20C).

Notably, production of the CD47-hCD19 CARs using bi-cistronic vectors resulted in favorable physical and functional titers for the constructs tested, as shown by genome quantification assay (FIG. 21A), functional titer (FIG. 218), and calculated particle-to-infectivity ratios (FIG. 21C).

### Transduction Titration

The threshold of lentiviral vector dosing to produce CD47-huCD19 CAR T cells was determined as follows.

On day 0, pan T cells were thawed in Complete CTS OpTmizer media (with 100 IU/mL IL-2) with CD4 and CD8 T cells thawed and cultured separately. That afternoon, T cells were stimulated with CTS Dynabeads at a 1:1 bead:cell ratio. The next morning (day 1), 5×10′ cells (50:50 ratio of CD4:CD8 cells) were added into 100 μL Complete CTS OpTmizer media (containing 100 IU/mL IL-2) in each well of a 96-well plate. Cells were then transduced (LVV comprising CD47-FMC63 (FIG. 19A), CD47-CAR400 VHVL (FIG. 19B), or CD47-CAR400 VLVH (FIG. 19C)) by spinoculation at 32° C. for 60 min at 1000×g at a range of doses (0 IU/cell, 0.781 IU/cell, 1.563 IU/cell. 3.125 IU/cell, 6.25 IU/cell, 12.5 IU/cell, 25 IU/cell, or 50 IU/cell). Two days later, cells were debeaded and split. One week after start of the experiment, cells were split for assessment of integration (VCN measured by ddPCR as described above) and flow panel analysis.

For flow panel analysis, cells were transferred to a 96-well round bottom plate and centrifuged at 750×g for 3 min. The supernatant was removed, and cells were washed with 200 μL PBS/well, and then centrifuged at 750×g for 3 min. The supernatant was removed, and 100 μL of the staining master mix (per 100 μL: 1:1000 V808 anti-L-D, 2 μL AF647 anti-CD47, 1 μL PE anti-soluble CD19) was added to each well. The plate was incubated at 4° C. for 45 min, and then centrifuged at 750×g for 3 min. The supernatant was removed, and cells were washed with 200 μl PBS. The plate was then centrifuged at 750×g for 3 min, and then the cells were washed again with 200 μL PBS. The plate was again centrifuged at 750×g for 3 min, and the supernatant was removed. The cells were then resuspended in 100 μL/well 1% paraformaldehyde in PBS, and samples were analyzed on a Cytoflex LX (Beckman Coulter).

Primary T cells were efficiently transduced with all three constructs (CD47-FMC63, CD47-CAR400 VHVL, and CD47-CAR400 VLVH) at low IU/cell concentrations, showing a dose saturation at 12.5 IU/cell (FIGS. 22A and 225). Confirmation of the transduction efficiency measured by flow cytometry was confirmed by ddPCR for bulk T cell population (FIG. 23A) or normalized to percent CD19CAR+ T cells FIG. 23B). At LVV doses under 12.5 IU/cell, the measured delU3 VCN of CAR-transduced T cells was less than 5/CAR+ cell.

In vitro characterization of hypoimmune CD47-hCD19 CAR T cells

Characterization of hypoimmune CD47-hCD19 CAR-T cell cytotoxic effects was performed as follows: Target cells (NALM-6 and NALM-6 CD19 knockout tumor cells; Raji and Raji CD19 knockout tumor cells; K562-CD19 (K562 engineered to express CD19 using LW) and parental K562 tumor cells) were counted and plated in a 96-well round bottom plate at 20,000 cells/well. NALM-6 cells were maintained in RPMI-1640+10% FBS. Target cell lines were transduced with ffluc. Effector cells (hypoimmune CAR T cells comprising CD47-FMC63, CD47-CAR400 VHVL, or CD47-CAR400 VLVH) were plated in wells with target cells at effector cell:target cell ratios (E:T ratios) of 1:1, 1:2, 1:4, 1:8, 1:16, and 1:32. After a 24-hour incubation, cells were spun down and the supernatant was collected for cytokine analysis. Cells were transferred to a 96-black well clear bottom plate and an equal volume of Bright-Glo luciferase (Promega) was added to cells and luminescence was measured on a SpectraMax reader (Molecular Devices).

All three constructs, CD47-FMC63, CD47-CAR400 VHVL, and CD47-CAR400 VLVH, demonstrated effective killing of NALM-6 tumor cells in vitro at all tested effector cell:target cell ratios (FIG. 24A). Notably, CD47-CAR400 VHVL and CD47-CAR400 VLVH hypoimmune CAR T cells demonstrated more effective NALM-6 cell killing than CD47-FMC63 hypoimmune CAR T cells at an E:T ratio of 1:32, with a NALM-6 survival percentage of less than 20% for both of the CD47-CAR400 VHVL and CD47-CAR400 VLVH hypoimmune CAR T cells. CD47-CAR400 VHVL and CD47-CAR400 VLVH hypoimmune CAR T cells also demonstrated cytokine levels that were elevated relative to the CD47-FMC63 hypoimmune CAR T cells, as well as a dose-dependent reduction in IFNγ, GM-CSF, IL-2, and TNFα (FIGS. 24B-24E, respectively). Tumor cell killing was markedly decreased when the hypoimmune CAR T cells were cultured with NALM-6 CD19 knockout cells (FIG. 24F), and levels of IFNγ, GM-CSF, IL-2, and TNFα were also comparable to levels measured in mock treated cells (FIGS. 240-24J). Similar cytotoxic effects for all three CD47-hCD19 hypoimmune CAR T cells were measured in Raji and Raji CD19 knockout tumor cells (FIGS. 25A-25J) and K562-CD19 and K562 tumor cells (FIGS. 26A-26J).

In vitro characterization of CD19 CAR-T cytotoxic effects by live cell imaging was to performed as follows: Target cells (NALM-6 and NALM-6 CD19 knockout iRFP713 cells) were counted and plated in a 96-well flat bottom plate at 20,000 cells/well. Effector cells (CD47-FMC63, CD47-CAR400 VHVL, or CD47-CAR400 VLVH hypoimmune CAR T cells) were plated in wells with target cells at an effector cell:target cell ratios (E:T ratio) of 1:1. Cells were imaged every 4 hours for 5 days on a Sartorius IncuCyte instrument. After 24 hours, 50 μL of supernatant was removed for cytokine analysis.

FIGS. 27A-27C show the cytotoxic effects of hypoimmune CAR T cells generated from three different donors. CAR T cell cytotoxicity was consistent across the different donors, with CD47-CAR400 VHVL and CD47-CAR400 VLVH showing greater cytotoxic effects than CD47-FMC63 in two of the three tested donors. Both CD47-CAR400 VHVL and CD47-CAR400 VLVH demonstrated effective control of tumor cell growth over the five-day study period. FIGS. 27D-27F show T cell expansion over the course of the study, with CAR T cells generated from three different donors. Both CD47-CAR400 VHVL and CD47-CAR400 VLVH hypoimmune CAR T cells demonstrated a steady expansion over the five-day study period. When the hypoimmune CAR T cells generated from three different donors were evaluated against NALM-6 CD19 knockout cells (FIGS. 28A-28F), little, if any, control of NALM-6 CD19 knockout tumor growth was measured (FIGS. 28A-28C), and no appreciable T cell expansion was measured (FIGS. 28D-28F).

FIGS. 29A-29D show levels of GM-CSF, IFNγ, IL-2, and TNFα, respectively, after 24 hours of incubation of the hypoimmune CAR T cells with NALM-6 and NALM-6 CD19 knockout cells. Both CD47-CAR400 VLVH and CD47-CAR400 VHVL hypoimmune CAR T cells induced slightly higher mean cytokine levels for all tested cytokines relative to the CD47-FMC63 hypoimmune CAR T cells when cultured with the wild type NALM-6 tumor cells. When the hypoimmune CAR T cells were cultured with the NALM-6 CD19 knockout cells there was little, if any, cytokine production.

### In Vivo Assessment of Hypoimmune CD47-hCD19 CARs

This example describes methods used to characterize and efficacy results of hypoimmune CD47-hCD19 CAR constructs in a B-cell tumor animal model. Table 36 provides an overview of the different experimental groups for the in vivo study.

In vivo characterization of CD19 CAR efficacy was performed as follows: Four days prior to injection with the hypoimmune CAR T cells, 6-12 week old NOD.Cg-Prkdc^{scid}ll2rg^{tm1Wjl}/SzJ (NSG) mice (The Jackson Laboratory) were intravenously injected with Nalm6:Wasabi-ffLuc cells (2.5×10⁵ cells/mouse) according to the experimental groups outlined in Table 36. Live imaging was performed one day later. Four days after injection of the Nalm6 tumor cells, mice were intravenously injected with CD47-FMC63 hypoimmune CAR T cells (at doses of 4×10s effector cells/mouse and 1×10⁶ effector cells/mouse), CD47-CAR400 (VLVH) hypoimmune CAR T Cells (at doses of 4×10⁶ effector cells/mouse and 1×10⁰ effector cells/mouse), CD47-CAR400 (VHVL) hypoimmune CAR T Cells (at doses of 4×10 effector cells/mouse and 1×10′ effector cells/mouse), or mock (untransduced PBMCs), according to the experimental groups outlined in Table 36. In vivo bioluminescent Imaging was performed on day ~3, 0, 5, 11, 19, 26, 28, 33, 40, and 47 of the study. In vivo live imaging was performed at the indicated timepoints using an IVIS in vivo imaging instrument (Perkin-Elmer). In vivo live imaging measured bioluminescence via intraperitoneal injection of D-luciferin substrate (Perkin-Elmer) All images were analyzed using Living Image software (Perkin-Elmer). Blood samples were collected on day 13 and 31 for flow cytometry to measure levels of circulating CAR+ cells and to measure maximum fluorescence intensity (MFI) of CD47. Survival of CAR-T treated mice was measured and reported using Kaplan-Meier curves. All in vivo animal studies were conducted in compliance with Institutional Animal Care and Use Committee (IACUC) approved protocols.

Dose dependent tumor suppression, as measured by flux, was observed in hypoimmune CAR T cells generated from donors 1 and 3 (FIGS. 30A and 30C), whereas there was little difference between administered doses of hypoimmune CAR T cells generated from donor 2 by day 40 (FIG. 30B). Both the VLVH and VHVL orientation of the CD47-CAR400 hypoimmune CAR T cell demonstrated increased tumor suppression relative to the CD47-FMC63 hypoimmune CAR T cells by day 40 (FIGS. 30A and 308). It was further noted that the CD47-FMC63 hypoimmune CAR T cells demonstrated delayed tumor suppression in donor 1 (high dose only) and donor 2 (both doses). All animals receiving hypoimmune CAR T cells show sustained tumor control compared to Mock or vehicle control mice. Animals who received low doses of hypoimmune CAR T cells generated from donor 3 (all three constructs) began to show high tumor burden comparable to Mock or vehicle control mice at day 47. Mock and vehicle treated animals were euthanized at day 27 due to moribund symptoms and high tumor burden.

Animals receiving CD47-CAR400 VLVH and CD47-CAR400 VHVL hypoimmune CAR T cells demonstrated increased numbers of CAR+ cells in circulating blood relative to animals receiving CD47-FMC63 hypoimmune CAR T cells (FIGS. 31A-31C). Levels of circulating CAR+ cells decreased from day 13 to 31 for all groups. Additionally, there was no significant expansion or regression of CD4+ or CD8+ T cells from day 13 to 31. CD47 overexpression, as measured by MFI, remained constant through at least 31 days following administration of the hypoimmune CAR T cells (FIGS. 32A-32C).

### Exemplary Embodiments

The different embodiments described in the sections below are suitable for use with one another (e.g., the chimeric antigen receptors described herein are suitable for use with the engineered cells described herein).

### Embodiments Related to Engineered Cells

The engineered cells described herein are suitable for use with the other embodiments described herein, including, but not limited to, gene editing embodiments and chimeric antigen receptor embodiments.
1. An engineered cell comprising one or more modifications that (i) reduce expression of one or more MHC class I molecules and/or one or more MHC class 1i molecules, and/or (ii) increase expression of one or more tolerogenic factors, wherein the reduced expression of (i) and the increased expression of (ii) is relative to a cell of the same cell type that does not comprise the modifications.
2. The engineered cell of embodiment 1, wherein the one or more modifications in (i) reduce expression of:
   a. one or more MHC class I molecules
   b. one or more MHC class II molecules; or
   c. one or more MHC class I molecules and one or more MHC class II molecules.
3. The engineered cell of embodiment 1 or embodiment 2, wherein the one or more modifications in (i) reduce expression of one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY—B, NFY-C, and any combination thereof.
4. The engineered cell of embodiment 3, wherein the engineered cell does not express one or more molecules selected from the group consisting of B2M, TAP I, NLRC5, CIITA, HLA-A, HLA-B, HLA-C, HLA-DP, HLA-DO, HLA-DR, HLA-DM, HLA-DO, RFX5, RFXANK, RFXAP, NFY-A, NFY—B, NFY-C, and combinations thereof.
5. The engineered cell of any of embodiments 1-4, wherein the one or more modifications that increase expression comprise increased cell surface expression, and/or the one or more modifications that reduce expression comprise reduced cell surface expression.
6. The engineered cell of any of embodiments 1-5, wherein the one or more modifications in (i) reduce expression of one or more MHC class I molecules.
7. The engineered cell of any of embodiments 1-6, wherein the one or more modifications in (i) reduce expression of B2M.
8. The engineered cell of any of embodiments 1-7, wherein the one or more modifications in (i) reduce expression of HLA-A, HLA-B, and/or HLA-C.
9. The engineered cell of any of embodiments 1-8, wherein the one or more modifications in (i) reduce expression of one or more MHC class II molecules.
10. The engineered cell of any of embodiments 1-9, wherein the one or more modifications in (i) reduce expression of CIITA.
11. The engineered cell of any of embodiments 1-10, wherein the one or more modifications in (i) reduce expression of HLA-DM, HLA-DO, HLA-DP, HLA-DQ, HLA-DR, RFX5, RFXANK, and/or RFXAP.
12. The engineered cell of any of embodiments 1-11, wherein the one or more tolerogenic factors comprise one or more tolerogenic factors selected from the group consisting of A20/TNFAIP3, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3, HLA-C, HLA-E, HLA-E heavy chain, HLA-F, HLA-G. ID01, IL-10, IL15-RF, IL-35, MANF, Mfge8, PD-L1, Serpinb9, and any combination thereof.
13. The engineered cell of any of embodiments 1-12, wherein the one or more tolerogenic factors comprise CD47.
14. The engineered cell of any of embodiments 1-13, wherein the one or more tolerogenic factors comprise CCL22.
15. The engineered cell of any of embodiments 1-14, wherein the one or more tolerogenic factors comprise CD16 or CD16 Fc receptor.
16. The engineered cell of any of embodiments 1-15, wherein the one or more tolerogenic factors comprise CD24.
17. The engineered cell of any of embodiments 1-16, wherein the one or more tolerogenic factors comprise CD39.
18. The engineered cell of any of embodiments 1-17, wherein the one or more tolerogenic factors comprise CR1.
19. The engineered cell of any of embodiments 1-18, wherein the one or more tolerogenic factors comprise CD52.
20. The engineered cell of any of embodiments 1-19, wherein the one or more tolerogenic factors comprise CD55.
21. The engineered cell of any of embodiments 1-20, wherein the one or more tolerogenic factors comprise CD200.
22. The engineered cell of any of embodiments 1-21, wherein the one or more tolerogenic factors comprise DUX4.
23. The engineered cell of any of embodiments 1-22, wherein the one or more tolerogenic factors comprise HLA-E.
24. The engineered cell of any of embodiments 1-23, wherein the one or more tolerogenic factors comprise HLA-G.
25. The engineered cell of any of embodiments 1-24, wherein the one or more tolerogenic factors comprise IDO1.
26. The engineered cell of any of embodiments 1-25, wherein the one or more tolerogenic factors comprise IL15-RF.
27. The engineered cell of any of embodiments 1-26, wherein the one or more tolerogenic factors comprise IL35.
28. The engineered cell of any of embodiments 1-27, wherein the one or more tolerogenic factors comprise PD-L1.
29. The engineered cell of any of embodiments 1-28, wherein the one or more tolerogenic factors comprise MANF.
30. The engineered cell of any of embodiments 1-29, wherein the one or more tolerogenic factors comprise A20/TNFAIP3.
31. The engineered cell of any of embodiments 1-30, wherein the one or more tolerogenic factors comprise HLA-E and CD47.
32. The engineered cell of any of embodiments 1-31, wherein the one or more tolerogenic factors comprise two or more tolerogenic factors selected from the group consisting of CD47, CD46, and CD59, optionally wherein the one or more tolerogenic factors comprise CD47. CD46, and CD59.
33. The engineered cell of any of embodiments 1-32, wherein the one or more tolerogenic factors comprise two or more tolerogenic factors selected from the group consisting of CD47 and CD39, optionally wherein the one or more tolerogenic factors comprise CD47 and CD39.
34. The engineered cell of any of embodiments 1-33, wherein the one or more tolerogenic factors comprise two or more tolerogenic factors selected from the group consisting of C047 and CCL22, optionally wherein the one or more tolerogenic factors comprise CD47 and CCL22.
35. The engineered cell of any of embodiments 1-34, wherein the one or more tolerogenic factors comprise two or more tolerogenic factors selected from the group consisting of CD47, HLA-G and PD-L1, optionally wherein the one or more tolerogenic factors comprise CD47 and PD-L1, and optionally wherein the one or more tolerogenic factors comprise CD47, HLA-G and PD-1.
36. The engineered cell of any of embodiments 1-35, wherein the one or more tolerogenic factors comprise two or more tolerogenic factors selected from the group consisting of CD24, CD47, and PD-L1, optionally wherein the one or more tolerogenic factors comprise CD24, CD47, and PD-1.
37. The engineered cell of any of embodiments 1-36, wherein the one or more tolerogenic factors comprise two or more tolerogenic factors selected from the group consisting of HLA-E, CD24, CD47, and PD-L1, optionally wherein the one or more tolerogenic factors comprise HLA-E, CD24, CD47, and PD-L1.
38. The engineered cell of any of embodiments 1-37, wherein the one or more tolerogenic factors comprise two or more tolerogenic factors selected from the group consisting of CD46, CD55, CD59, and CR1, optionally wherein the one or more tolerogenic factors comprise CD46. CD55, CD59, and CR1.
39. The engineered cell of any of embodiments 1-38, wherein the one or more tolerogenic factors comprise two or more tolerogenic factors selected from the group consisting of HLA-E, CD46, CD5S, CD59, and CR1, optionally wherein the one or more tolerogenic factors comprise HLA-E, CD46, CD55, CD59, and CR1.
40. The engineered cell of any of embodiments 1-39, wherein the one or more tolerogenic factors comprise two or more tolerogenic factors selected from the group consisting of HLA-E, CD24, CD47, PD-L1, CD46, CD55, CD59, and CR1, optionally wherein the one or more tolerogenic factors comprise HLA-E, CD24, CD47, PD-L1, CD46, CD55, CD59, and CR1.
41. The engineered cell of any of embodiments 1-40, wherein the one or more tolerogenic factors comprise HLA-E and PD-L1.
42. The engineered cell of any of embodiments 1-41, wherein the one or more tolerogenic factors comprise two or more tolerogenic factors selected from the group consisting of HLA-E, PD-L1, and A20/TNFAIP, optionally wherein the one or more tolerogenic factors comprise HLA-E, PD-L1, and A20/TNFAIP.
43. The engineered cell of any of embodiments 1-42, wherein the one or more tolerogenic factors comprise two or more tolerogenic factors selected from the group consisting of HLA-E, PD-L1, and MANF, optionally wherein the one or more tolerogenic factors comprise HLA-E, PD-L1, and MANF.
44. The engineered cell of any of embodiments 1-43, wherein the one or more tolerogenic factors comprise two or more tolerogenic factors selected from the group consisting of HLA-E, PD-L1, A20/TNFAIP, and MANF, optionally wherein the one or more tolerogenic factors comprise HLA-E, PD-L1, A20/TNFAIP, and MANF.
45. An engineered cell comprising one or more modifications that (i) reduce expression of one or more MHC class I molecules and one or more MHC class II molecules, and (ii) increase expression of CD47, wherein the reduced expression of (i) and the increased expression of (ii) is relative to a cell of the same cell type that does not comprise the modifications.
46. The engineered cell of embodiment 45, wherein the one or more modifications in (i) reduce expression of one or more molecules selected from the group consisting of B2M, TAP I, NLRC5, CIITA, HLA-A, HLA-B, HLA-C, HLA-DP, HLA-00, HLA-DR, HLA-DM, HLA-DO, RFX5, RFXANK, RFXAP, NFY-A, NFY—B, NFY-C, and any combination thereof.
47. The engineered cell of embodiment 45 or embodiment 46, wherein the one or more modifications in (i) reduce expression of B2M.
48. The engineered cell of any of embodiments 45-47, wherein the one or more modifications in (i) reduce expression of HLA-A, HLA-B, and/or HLA-C.
49. The engineered cell of any of embodiments 45-48, wherein the one or more modifications in (i) reduce expression of CIITA.
50. The engineered cell of any of embodiments 45-48, wherein the one or more modifications in (i) reduce expression of HLA-DP, HLA-DR, and/or HLA-DQ.
51. The engineered cell of any of embodiments 1-50, wherein the engineered cell 1i further comprises one or more modifications that increase expression of one or more additional tolerogenic factors.
52. The engineered cell embodiment 51, wherein the one or more additional tolerogenic factors comprise one or more tolerogenic factors selected from the group consisting of A20/TNFAIP3, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3, HLA-C, HLA-E, HLA-E heavy chain. HLA-F, HLA-G, IDO1, IL-10, IL15-RF, IL-35, MANF, Mfge8, PD-L1, Serpinb9, and any combination thereof.
53. The engineered cell of embodiment 52, wherein the one or more additional tolerogenic factors comprise CD47.
54. The engineered cell of any one of embodiments 1-57, wherein the engineered cell further comprises one or more modifications that reduce expression of one or more additional molecules.
55. The engineered cell of embodiment 54, wherein the one or more additional molecules comprises B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY—B, NFY-C, ABO, CADM1, CD58, CD38, CD142, CD155, CEACAMI, CTLA-4, FUT1, ICAM1, IRF1, MIC-A, MIC-B, NLGN4Y, PCODH11Y, PD-1, a protein that is involved in oxidative or ER stress, RHD, TRAC, TRB, optionally wherein the protein that is involved in oxidative or ER stress is selected from the group consisting of TXNIP. PERK, IRE1α, and DJ-1 (PARK7).
56. The engineered cell of embodiment 54 or 55, wherein the one or more additional molecules comprise one or more Y chromosome proteins, optionally Protocadherin-11 Y-linked (PCDH1IY) and/or Neuroligin-4 Y-linked (NLGN4Y).
57. The engineered cell of any of embodiments 54-57, wherein the one or more additional molecules comprise one or more NK cell ligands, optionally MIC-A and/or MIC-B.
58. The engineered cell of any of embodiments 54-57, wherein the one or more additional molecules comprise one or more proteins involved in oxidative or ER stress, optionally thioredoxin-interacting protein (TXNIP), PKR-like ER kinase (PERK), inositol-requiring enzyme 1a (IRE1a), and/or DJ-1 (PARK7).
59. The engineered cell of any of embodiments 54-58, wherein the one or more additional molecules comprise one or more blood antigen proteins, optionally ABO, FUT1 and/or RHD.
60. The engineered cell of any one of embodiments 1-59, wherein the engineered cell further comprises one or more modifications that reduce expression of 82M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY—B, NFY-C, ABO, CADM1, CD58, CD38, CD142, CD155, CEACAMI, CTLA-4, FUT1, ICAM1. IRF1, MIC-A, MIC-8, NLGN4Y, PCDH11Y, PD-1, a protein that is involved in oxidative or ER stress, RHD, TRAC, TRB, optionally wherein the protein that is involved in oxidative or ER stress is selected from the group consisting of TXNIP, PERK, IRE1α, and DJ-1 (PARK7).
61. The engineered cell of embodiment 60, wherein TRB is TRBC1, TRBC2, or TRBC1 and TRBC2.
62. The engineered cell of any of embodiments 1-61, wherein reduced expression comprises no cell surface expression or no detectable cell surface expression.
63. The engineered cell of any of embodiments 1-62, wherein reduced expression comprises reduced mRNA expression, optionally wherein reduced expression comprises no detectable mRNA expression.
64. The engineered cell of any of embodiments 1-63, wherein reduced expression comprises reduced protein expression or reduced protein activity, optionally wherein reduced expression comprises no detectable protein expression or protein activity.
65. The engineered cell of any of embodiments 1-64, wherein reduced expression comprises eliminating activity of a gene encoding or regulating the expression of i) the one or more MHC class I molecules and/or the one or more MHC class II molecules, or ii) the one or more additional molecules.
66. The engineered cell of any of embodiments 1-65, wherein reduced expression comprises inactivation or disruption of an allele of a gene encoding or regulating the expression of i) the one or more MHC class I molecules and/or the one or more MHC class II molecules, or ii) the one or more additional molecules.
67. The engineered cell of any of embodiments 1-66, wherein reduced expression comprises inactivation or disruption of both alleles of a gene encoding or regulating the expression of i) the one or more MHC class I molecules and/or the one or more MHC class II molecules, or ii) the one or more additional molecules.
68. The engineered cell of any of embodiments 1-67, wherein the one or more modifications to reduce expression comprises an indel in a gene encoding or regulating the expression of i) the one or more MHC class I molecules and/or the one or more MHC class II molecules, or ii) the one or more additional molecules.
69. The engineered cell of any of embodiments 1-68, wherein the one or more modifications to reduce expression comprises a frameshift mutation or a deletion of a contiguous stretch of genomic DNA of a gene encoding or regulating the expression of i) the one or more MHC class I molecules and/or the one or more MHC class II molecules, or ii) the one or more additional molecules.
70. The engineered cell of any of embodiments 1-69, wherein the one or more modifications to reduce expression comprises inactivation or disruption of all coding sequences of a gene encoding or regulating the expression of i) the one or more MHC class I molecules and/or the one or more MHC class II molecules, or ii) the one or more additional molecules.
71. The engineered cell of any of embodiments 1-69, wherein the one or more modifications to reduce expression comprises knocking out a gene encoding or regulating the expression of i) the one or more MHC class I molecules and/or the one or more MHC class II molecules, or ii) the one or more additional molecules.
72. The engineered cell of any of embodiments 1-71, wherein the engineered cell comprises one or more modifications that:
   a. reduce expression of MHC class I and/or MHC class II molecules;
   b. increase expression of CD47; and
   c. increase expression of CCL22.
73. The engineered cell of any of embodiments 1-71, wherein the engineered cell comprises one or more modifications that:
   a. reduce expression of MHC class I and/or MHC class II molecules;
   b. increase expression of CD47; and
   c. increase expression of CD39.
74. The engineered cell of any of embodiments 1-71, wherein the engineered cell comprises one or more modifications that:
   a. reduce expression of MHC class I and/or MHC class II molecules;
   b. increase expression of CD47; and
   c. increase expression of CD46 and CD59.
75. The engineered cell of any of embodiments 1-71, wherein the engineered cell comprises one or more modifications that:
   a. reduce expression of MHC class I and/or MHC class II molecules;
   b. increase expression of CD47; and
   c. increase expression of PD-L1.
76. The engineered cell of any of embodiments 1-71, wherein the engineered cell comprises one or more modifications that:
   a. reduce expression of MHC class I and/or MHC class II molecules;
   b. increase expression of CD47; and
   c. increase expression of HLA-G and PD-L1
77. The engineered cell of any of embodiments 1-71, wherein the engineered cell comprises one or more modifications that:
   a. reduce expression of MHC class I and/or MHC class II molecules;
   b. increase expression of CD47; and
   c. reduced expression of CD142 (TF).
78. The engineered cell of any of embodiments 1-71, wherein the engineered cell comprises one or more modifications that:
   a. reduce expression of MHC class I and/or MHC class II molecules;
   b. increase expression of CD47; and
   c. reduced expression of MIC-A and/or MIC-B.
79. The engineered cell of any of embodiments 1-71, wherein the engineered cell comprises one or more modifications that:
   a. reduce expression of MHC class I and/or MHC class II molecules; and
   b. increase expression of CD24.
80. The engineered cell of any of embodiments 1-71, wherein the engineered cell comprises one or more modifications that:
   a. reduce expression of MHC class I and/or MHC class II molecules; and
   b. increase expression of CD200.
81. The engineered cell of any of embodiments 1-71, wherein the engineered cell comprises one or more modifications that:
   a. reduce expression of MHC class I and/or MHC class II molecules; and
   b. increase expression of CD52.
82. The engineered cell of any of embodiments 1-71, wherein the engineered cell comprises one or more modifications that:
   a. reduce expression of MHC class I and/or MHC class II molecules; and
   b. increase expression of DUX4.
83. The engineered cell of any of embodiments 1-71, wherein the engineered cell comprises one or more modifications that:
   a. reduce expression of MHC class I and/or MHC class II molecules; and
   b. increase expression of IDO1.
84. The engineered cell of any of embodiments 1-71, wherein the engineered cell comprises one or more modifications that:
   a. reduce expression of MHC class I and/or MHC class II molecules; and
   b. increase expression of IL-35.
85. The engineered cell of any of embodiments 1-71, wherein the engineered cell comprises one or more modifications that
   a. reduce expression of MHC class I and/or MHC class II molecules; and
   b. increase expression of PD-1.
86. The engineered cell of any of embodiments 1-71, wherein the engineered cell comprises one or more modifications that:
   a. reduce expression of MHC class I and/or MHC class II molecules; and
   b. increase expression of HLA-E.
87. The engineered cell of any of embodiments 1-71, wherein the engineered cell comprises one or more modifications that:
   a. reduce expression of MHC class I and/or MHC class II molecules; and
   b. increase expression of HLA-G.
88. The engineered cell of any of embodiments 1-71, wherein the engineered cell comprises one or more modifications that:
   a. reduce expression of MHC class I and/or MHC class II molecules;
   b. reduce expression of CD155; and is c, increase expression of HLA-E.
89. The engineered cell of any of embodiments 1-71, wherein the engineered cell comprises one or more modifications that
   a. reduce expression of MHC class I molecules;
   b. reduce expression of RFXANK;
   c. increase expression of HLA-E.
90. The engineered cell of any of embodiments 1-71, wherein the engineered cell comprises one or more modifications that
   a. reduce expression of MHC class I and/or MHC class II molecules;
   b. reduce expression of MIC-A and/or MIC-B;
   c. increase expression of one or more of CD47. CD24 and PD-L1; and
   d. increase expression of CD46, CD55, CD59 and CR1.
91. The engineered cell of any of embodiments 1-71, wherein the engineered cell comprises one or more modifications that:
   a. reduce expression of MHC class I molecules;
   b. reduce expression of MIC-A and/or MIC-B;
   c. reduce expression of TXNIP; and
   d. increase expression of PD-L1 and HLA-E.
92. The engineered cell of embodiment 90, wherein the modifications further increase expression of A20/TNFAIP3 and MANF.
93. The engineered of any one of embodiments 1-92, wherein the one or more modifications that reduce expression of MHC class I and/or MHC class II molecules consist of one or more modifications that reduce expression of MHC class I molecules.
94. The engineered of any one of embodiments 1-92, wherein the one or more modifications that reduce expression of MHC class I and/or MHC class II molecules consist of one or more modifications that reduce expression of MHC class II molecules.
95. The engineered of any one of embodiments 1-92, wherein the one or more modifications that reduce expression of MHC class I and/or MHC class II molecules consist of one or more modifications that reduce expression of MHC class I molecules and MHC class II molecules.
96. The engineered cell of embodiment 1-95, wherein increased expression comprises increased mRNA expression.
97. The engineered cell of embodiment 1-96, wherein increased expression comprises increased protein expression or protein activity.
98. The engineered cell of any one of embodiments 1-97, wherein increased expression comprises increasing activity of a gene encoding or regulating the expression of i) the one or more tolerogenic factors, or ii) the one or more additional tolerogenic factors.
99. The engineered cell of embodiment 98, wherein the gene is an endogenous gene and the one or more modifications comprise one or more modifications of an endogenous promoter.
100. The engineered cell of embodiment 98, wherein the gene is an endogenous gene and the one or more modifications comprise introduction of a heterologous promoter.
101. The engineered cell of embodiment 100, wherein the heterologous promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.
102. The engineered cell of any of embodiments 1-94, wherein the engineered cell comprises one or more transgenes.
103. The engineered cell of embodiment 102, wherein the one or more transgenes encode at least one of the one or more tolerogenic factors or the one or more additional tolerogenic factors.
104. The engineered cell of embodiment 102 or 103, wherein the one or more transgenes encode at least one of the one or more additional tolerogenic factors.
105. The engineered cell of any one of embodiments 102-104, wherein the one or more transgenes encode one or more additional molecules.
106. The engineered cell of any of embodiments 102-105. wherein the one or more transgenes comprise one or more regulatory elements.
107. The engineered cell of any of embodiments 102-106, wherein the one or more transgenes are operably linked to the one or more regulatory elements.
108. The engineered cell of embodiment 106 or embodiment 107, wherein the one or more regulatory elements comprise one or more promoters, enhancers, introns, terminators, translation initiation signals, polyadenylation signals, replication elements, RNA processing and export elements, transposons, transposases, insulators, internal ribosome entry sites (IRES), 5′UTRs, 3′UTRs, mRNA 3′end processing sequences, boundary elements, locus control regions (LCR), matrix attachment regions (MAR), recombination or cassette exchange sequences, linker sequences, secretion signals, resistance markers, anchoring peptides, localization signals, fusion tags, affinity tags, chaperonins, and proteases.
109. The engineered cell of any of embodiments 102-109. wherein the promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.
110. The engineered cell of any of embodiments 102-109, wherein the engineered cell comprises one or more vectors encoding the one or more transgenes.
111. The engineered cell of embodiment 110, wherein at least one of the one or more vectors is a polycistronic vector.
112. The engineered cell of embodiment 111, wherein the polycistronic vector encodes at least one of the one or more tolerogenic factors or the one or more additional tolerogenic factors.
113. The engineered cell of embodiment 111 or embodiment 112, wherein the polycistronic vector further encodes at least one of the one or more tolerogenic factors or the one or more additional tolerogenic factors.
114. The engineered cell of embodiment of embodiment 112 or embodiment 113, wherein the polycistronic vector further encodes at least one of the one or more additional molecules.
115. The engineered cell of any one of embodiments 102-114, wherein the one or more transgenes are separated by one or more linker sequences.
116. The engineered cell of embodiment 115, wherein the one or more linker sequences comprise an IRES sequence or a cleavable peptide sequence 117. The engineered cell of embodiment 116, wherein the cleavable peptide sequence comprises a self-cleavable peptide, optionally a 2A peptide.
118. The engineered cell of embodiment 117, wherein the 2A peptide is selected from the group consisting of a F2A sequence, an E2A sequence, a P2A sequence, and a T2A sequence.
119. The engineered cell of any of embodiments 116-118, wherein the cleavable peptide sequence comprises a protease cleavable sequence or a chemically cleavable sequence.
120. The engineered cell of any of embodiments 112-119, wherein at least two of the one or more tolerogenic factors, the one or more additional tolerogenic factors, and/or the one or more additional molecules are operably linked to the same promoter.
121. The engineered cell of any of embodiment 120, wherein the promoter is a constitutive promoter.
122. The engineered cell of embodiment 120 or 121, wherein the promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter. tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

### Embodiments Related to CD19 CARs

123. The engineered cell of any of embodiments 105-122, wherein the one or more additional molecules comprise a chimeric antigen receptor (CAR).
124. The engineered cell of embodiment 123, wherein the CAR comprises a signal peptide, an extracellular binding domain specific to CD19, a hinge domain, a transmembrane domain, an intracellular costimulatory domain, and/or an intracellular signaling domain.
125. The engineered cell of embodiment 123 or embodiment 124, wherein the CAR is bispecific for CD19 and any one of CDS, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3. HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, G_{D2}, G_{D3}. HMW-MAA, IL-11Ra, KDR, Lewis Y, L1-cell adhesion molecule, MADE-Al, Oncofetal antigen (h5T4), TAG-72, CD19/22, Syndecan 1, or BCMA.
126. The engineered cell of embodiment 125, wherein the CAR is a CD19/CD5-bispecific CAR.
127. The engineered cell of embodiment 125, wherein the CAR is a CD19/CD19-bispecific CAR.
128. The engineered cell of embodiment 125, wherein the CAR is a CD19/CD20-bispecific CAR.
129. The engineered cell of embodiment 125, wherein the CAR is a CD19/CD22-bispecific CAR.
130. The engineered cell of embodiment 125, wherein the CAR is a CD19/CD23-bispecific CAR.
131. The engineered cell of embodiment 125, wherein the CAR is a CD19/CD30-bispecific CAR.
132. The engineered cell of embodiment 125, wherein the CAR is a CD19/CD33-bispecific CAR.
133. The engineered cell of embodiment 125. wherein the CAR Is a CD19/CD38-bispecific CAR.
134. The engineered cell of embodiment 125, wherein the CAR is a CD19/CD70-bispecific CAR.
135. The engineered cell of embodiment 125, wherein the CAR is a CD19/CD123-bispecific CAR.
136. The engineered cell of embodiment 125, wherein the CAR is a CD19/CD138-bispecific CAR.
137. The engineered cell of embodiment 125, wherein the CAR is a CD19/GPRC5D-bispecific CAR.
138. The engineered cell of embodiment 125, wherein the CAR is a CD19/LeY-bispecific CAR.
139. The engineered cell of embodiment 125, wherein the CAR is a CD19/NKG2D-bispecific CAR.
140. The engineered cell of embodiment 125, wherein the CAR is a CDI9/WT1-bispecific CAR.
141. The engineered cell of embodiment 125, wherein the CAR is a CD19/GD2-bispecific CAR.
142. The engineered cell of embodiment 125, wherein the CAR is a CD19/HER2-bispecific CAR.
143. The engineered cell of embodiment 125, wherein the CAR is a CD19/EGFR-bispecific CAR.
144. The engineered cell of embodiment 125, wherein the CAR is a CD19/EGFRvIII-bispecific CAR.
145. The engineered cell of embodiment 125, wherein the CAR is a CD19/67H3-bispecific CAR.
146. The engineered cell of embodiment 125. wherein the CAR Is a CD19/PSMA-bispecific CAR.
147. The engineered cell of embodiment 125, wherein the CAR is a CD19/PSCA-bispecific CAR.
148. The engineered cell of embodiment 125, wherein the CAR is a CD19/CAIX-bispecific CAR.
149. The engineered cell of embodiment 125, wherein the CAR is a CD19/CD171-bispecific CAR.
150. The engineered cell of embodiment 125, wherein the CAR is a CD19/CEA-bispecific CAR.
151. The engineered cell of embodiment 125, wherein the CAR is a CD19/CSPG4-bispecific CAR.
152. The engineered cell of embodiment 125, wherein the CAR is a CD19/EPHA2-bispecific CAR.
153. The engineered cell of embodiment 125, wherein the CAR is a CD19/FAP-bispecific CAR.
154. The engineered cell of embodiment 125, wherein the CAR is a CD19/FRa-bispecific CAR.
155. The engineered cell of embodiment 125, wherein the CAR is a CDI9/IL-13Rα-bispecific CAR.
156. The engineered cell of embodiment 125, wherein the CAR is a CD19/Mesothelin-bispecific CAR.
157. The engineered cell of embodiment 125, wherein the CAR is a CD19/MUC1-bispecific CAR.
158. The engineered cell of embodiment 125, wherein the CAR is a CD19/MUC16-bispecific CAR.
159. The engineered cell of embodiment 125. wherein the CAR Is a CD19/ROR1-bispecific CAR.
160. The engineered cell of embodiment 125, wherein the CAR is a CD19/C-Met-bispecific CAR.
161. The engineered cell of embodiment 125, wherein the CAR is a CD19/CD133-bispecific CAR.
162. The engineered cell of embodiment 125, wherein the CAR is a CD19/Ep-CAM-bispecific CAR. is 163. The engineered cell of embodiment 125, wherein the CAR is a CD19/GPC3-bispecific CAR.
164. The engineered cell of embodiment 125, wherein the CAR is a CD19/HPV16-bispecific CAR.
165. The engineered cell of embodiment 125, wherein the CAR is a CD19/IL13Ra2-bispecific CAR.
166. The engineered cell of embodiment 125, wherein the CAR is a CD19/MAGEA3-bispecific CAR.
167. The engineered cell of embodiment 125, wherein the CAR is a CD19/MAGEA4-bispecific CAR.
168. The engineered cell of embodiment 125, wherein the CAR is a CD19/MART1-bispecific CAR.
169. The engineered cell of embodiment 125, wherein the CAR is a CD19/NY-ESO-bispecific CAR.
170. The engineered cell of embodiment 125, wherein the CAR is a CD19/VEGFR2-bispecific CAR.
171. The engineered cell of embodiment 125, wherein the CAR is a CD19/α-Folate-bispecific CAR.
172. The engineered cell of embodiment 125, wherein the CAR is a CD19/CD24-bispecific CAR.
173. The engineered cell of embodiment 125, wherein the CAR is a CD19/CD44v7/8-bispecific CAR.
174. The engineered cell of embodiment 125, wherein the CAR is a CD19/EGP-2-bispecific CAR.
175. The engineered cell of embodiment 125, wherein the CAR is a CD19/EGP-40-bispecific CAR.
176. The engineered cell of embodiment 125, wherein the CAR is a CD19/erb-82-bispecific CAR.
177. The engineered cell of embodiment 125, wherein the CAR is a CD19/erb-8-bispecific CAR.
178. The engineered cell of embodiment 125, wherein the CAR is a CD19/FBP-bispecific CAR.
179. The engineered cell of embodiment 125, wherein the CAR is a CD19/Fetal acetylcholine e receptor-bispecific CAR.
180. The engineered cell of embodiment 125. wherein the CAR is a CD19/G_{D2}-bispecific CAR.
181. The engineered cell of embodiment 125, wherein the CAR is a CD19/G_{D3}-bispecific CAR.
182. The engineered cell of embodiment 125, wherein the CAR is a CD19/HMW-MAA-bispecific CAR.
183. The engineered cell of embodiment 125, wherein the CAR is a CD19/IL-11Rat-bispecific CAR.
184. The engineered cell of embodiment 125, wherein the CAR is a CD19/KDR-bispecific CAR.
185. The engineered cell of embodiment 125. wherein the CAR Is a CD19/Lewis Y-bispecific CAR.
186. The engineered cell of embodiment 125, wherein the CAR is a CD19/L1-cell adhesion molecule-bispecific CAR.
187. The engineered cell of embodiment 125, wherein the CAR is a CD19/MADE-A1-bispecific CAR.
188. The engineered cell of embodiment 125, wherein the CAR is a CD19/Oncofetal antigen (h5T4)-bispecific CAR.
189. The engineered cell of embodiment 125, wherein the CAR is a CD19/TAG-72-bispecific CAR.
190. The engineered cell of embodiment 125, wherein the CAR is a CD19/CD19/22-bispecific CAR.
191. The engineered cell of embodiment 125, wherein the CAR is a CD19/Syndecan-1-bispecific CAR.
192. The engineered cell of embodiment 125. wherein the CAR is a CD19/BCMA-bispecific CAR.
193. The engineered cell of any of embodiments 105-192. wherein the one or more additional molecules comprise one or more safety switches.
194. The engineered cell of embodiment 193, wherein the one or more safety switches are capable of controlled killing of the engineered cell.
195. The engineered cell of embodiment 193 or 194, wherein the one or more safety switches induce controlled cell death in the presence of a drug or prodrug, or upon activation by a selective exogenous compound.
196. The engineered cell of any of embodiments 193-195, wherein the one or more safety switches comprise is an inducible protein capable of inducing apoptosis of the engineered cell.
197. The engineered cell of embodiment 196, wherein the inducible protein capable of inducing apoptosis of the engineered cell is a caspase protein.
198. The engineered cell of embodiment 196, wherein the caspase protein is caspase 9.
199. The engineered cell of any of embodiments 193-198, wherein the one or more safety switches comprise one or more suicide genes.
200. The engineered cell of embodiment 199, wherein the one or more suicide genes are selected from the group consisting of cytosine deaminase (CyD), herpesvirus thymidine kinase (HSV-Tk), an inducible caspase 9 (iCaspase9), and rapamycin-activated caspase 9 (rapaCasp9).

### Embodiments Related to Gene Editing

201. The engineered cell of any of embodiments 102-200, wherein at least one of the one or more transgenes are integrated into the genome of the engineered cell.
202. The engineered cell of embodiment 201, wherein integration is by non-targeted insertion into the genome of the engineered cell.
203. The engineered cell of embodiment 202, wherein integration is by non-targeted insertion into the genome of the engineered cell using a lentiviral vector.
204. The engineered cell of embodiment 201, wherein integration is by targeted insertion into a target genomic locus of the engineered cell.
205. The engineered cell of embodiment 204, wherein targeted insertion is homology-directed repair.
206. The engineered cell of embodiment 204 or 205. wherein the target genomic locus is selected from the group consisting of an albumin gene locus, an ABO gene locus, a B2M gene locus, a CIITA gene locus, a CCR5 gene locus, a CD142 gene locus. a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPP1R12C (also known as AAVS1) gene locus, an RHO gene locus. a ROSA26 gene locus, a safe harbor gene locus. a SHS231 locus, a TAP1 gene locus, a TRAC gene locus, and a TRBC gene locus.
207. The engineered cell of any of embodiments 1-206, wherein the genome of the engineered cell comprises one or more gene edits in one or more genes encoding the one or more molecules of any of embodiments 1-206 having reduced expression.
208. The engineered cell of any of embodiments 1-207, wherein the engineered cell comprises a genome editing complex.
209. The engineered cell of embodiment 208, wherein the genome editing complex comprises ay and a genome modifying entity.
210. The engineered cell of embodiment 209, wherein the genome targeting entity localizes the genome editing complex to the target locus, optionally wherein the genome targeting entity is a nucleic acid-guided targeting entity.
211. The engineered cell of embodiment 209 or embodiment 210, wherein the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.
212. The engineered cell of any of embodiments 209-211. wherein the genome targeting entity is selected from the group consisting of Cas1. Cas2, Cas3, Cas4, Cas5, Cas6, Cas7, Cas8a, Cas8b, Cas8c, Cas9, Cas10, Cas12, Cas12a (Cpf1). Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5. Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2. Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.
213. The engineered cell of embodiment 209, wherein the genome modifying entity cleaves, deaminates, nicks, polymerizes, interrogates, integrates, cuts, unwinds, breaks, alters, methylates, demethylates, or otherwise destabilizes the target locus.
214. The engineered cell of embodiment 213, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.
215. The engineered cell of embodiment 213 or embodiment 214, wherein the genome modifying entity selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.
216. The engineered cell of any of embodiments 213-215, wherein the genome modifying entity is selected from the group consisting of Cas1, Cas2, Cas3, Cas4. Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4. Cas5e, Csf1, Csm1, Csm2. Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TETICD, histone acetyltransferase p300, histone methyltransferase SMYD3, histone methyltransferase PRDM9, H3K79 methyltransferase DOTIL, transcriptional repressor, or a functional portion thereof.
217. The engineered cell of any of embodiments 209-216, wherein the genome targeting entity and the genome modifying entity are different domains of a single polypeptide.
218. The engineered cell of any of embodiments 209-216, wherein the genome targeting entity and genome modifying entity are different polypeptides that are operably linked together.
219. The engineered cell of any of embodiments 209-216. wherein the genome targeting entity and genome modifying entity are different polypeptides that are not linked together.
220. The engineered cell of any of embodiments 209-216, wherein the genome editing complex comprises a guide nucleic acid having a targeting domain that is complementary to at least one target locus, optionally wherein the guide nucleic acid is a guide RNA (gRNA).
221. The engineered cell of any of embodiments 209-220, wherein the one or more modifications are made by the genome editing complex.
222. The engineered cell of embodiment 221, wherein the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the is gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).
223. The engineered cell of embodiment 221 or embodiment 222, wherein the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Cay1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).
224. The engineered cell of any of embodiments 221-223, wherein the modifications made by the genome editing complex are made using a guide RNA (gRNA) having a targeting domain that is complementary to at least one target site.

### Embodiments Related to Types of Cells for Making Engineered Cells

225. The engineered cell of any of embodiments 1-224. wherein the engineered cell is a human cell or an animal cell.
226. The engineered cell of embodiment 225, wherein the animal cell is a porcine cell, a bovine cell, or an ovine cell.
227. The engineered cell of embodiment 225, wherein the engineered cell is a human cell.
228. The engineered cell of any of embodiments 1-227, wherein the engineered cell is a stem cell or progenitor cell.
229. The engineered cell of embodiment 228, wherein the engineered cell is a differentiated cell derived from the stem cell or progenitor cell.
230. The engineered cell of embodiment 228 or 229, wherein the stem cell or progenitor cell is selected from the group consisting of an induced pluripotent stem cell, an embryonic stem cell, a hematopoietic stem cell, a mesenchymal stem cell, an endothelial stem cell, an epithelial stem cell, an adipose stem cell, a germline stem cell, a lung stem cell, a cord blood stem cell, a pluripotent stem cell (PSC), and a multipotent stem cell.
231. The engineered cell of any of embodiments 1-227, wherein the engineered cell is a differentiated cell derived from a pluripotent stem cell or a progeny thereof.
232. The engineered cell of embodiment 231, wherein the pluripotent stem cell is an induced pluripotent stem cell.
233. The engineered cell of any of embodiments 1-227, wherein the engineered cell is a primary cell isolated from a donor subject.
234. The engineered cell of embodiment 223, wherein the donor subject is healthy or is not suspected of having a disease or condition at the time the donor sample is obtained from the individual donor.
235. The engineered cell of any of embodiments 1-234, wherein the engineered cell is selected from the group consisting of an islet cell, a beta islet cell, a pancreatic islet cell, an immune cell, a B cell, a T cell, a natural killer (NK) cell, a natural killer T (NKT) cell, a macrophage cel, an endothelial cell, a muscle cell, a cardiac muscle cell, a smooth muscle cell, a skeletal muscle cell, a dopaminergic neuron, a retinal pigmented epithelium cell, an optic cell, a hepatocyte, a thyroid cell, a skin cell, a glial progenitor cell, a neural cell, a cardiac cell, a stem cell, a hematopoietic stem cell, an induced pluripotent stem cell (iPSC), a mesenchymal stem cell (MSC), an embryonic stem cell (ESC), a pluripotent stem cell (PSC), and a blood cell.
236. The engineered cell of any of embodiments 1-235, wherein the cell is ABO blood group type O.
237. The engineered cell of any of embodiments 1-238, wherein the cell comprises a functional ABO A allele and/or a functional ASO B allele.
238. The engineered cell of any of embodiments 1-237, wherein the cell is Rhesus factor negative (Rh—).
239. The engineered cell of any of embodiments 1-237, wherein the cel is Rhesus factor positive (Rh+).
240. A method of generating the engineered cell of any of embodiments 1-239 comprising
   a. obtaining a cell; and
   b. introducing the one or more modifications of any of embodiments 1-239 into the cell.
241. The method of embodiment 240, wherein the method further comprises selecting the engineered cell from a population of cells based on the presence of and/or level of one or more of the modifications.
242. The method of embodiment 240 or 241, wherein the cell is a stem cell or a progenitor cel and the method further comprises differentiating the stem cell or the progenitor ceN.
243. The method of embodiment 240 or 241, wherein the cell is a pluripotent stem cell or a progeny thereof and the method comprises differentiating the pluripotent stem cell or progeny thereof.
244. The method of embodiment 240 or 241 wherein the cell is a primary cell.

### Embodiments Relating to Engineered Cells II

245. The method of any of embodiments 240-244, wherein the method comprises introducing one or more gene edits into the genome of the cell.
246. The method of embodiment 245, wherein the one or more gene edits are introduced into the genome of the cell by non-targeted insertion.
247. The method of embodiment 245, wherein the one or more gene edits are introduced into the genome of the cell by targeted insertion.
248. The method of embodiment 245 or 247, wherein the one or more gene edits are introduced into one or more genes encoding the one or more molecules of any of embodiments 1-238.
249. The method of embodiment 248, wherein the engineered cell has increased expression of the one or more molecules encoded by the one or more edited genes.
250. The method of embodiment 248 or 249, wherein the engineered cell has reduced expression of the one or more molecules encoded by the one or more edited genes.
251. The method of any of embodiments 245-250, wherein the one or more gene edits are Introduced into the genome of cell using at least one of the genome editing complexes of any of embodiments 208-224.
252. The method of any of embodiments 245-251, wherein the one or more gene edits are introduced into the genome of cell at one or more target genomic loci selected from the group consisting of an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDMSD gene locus, an LRP1 gene locus, a MIC-A gene locus, a MIC-B gene locus, a PPPIR12C (also known as AAVS1) gene locus, an RHD gene locus, a ROSA26 gene locus, a safe harbor gene locus, a SHS231 locus, a TAP1 gene locus, a TRAC gene locus, and a TRBC gene locus.
253. An engineered cell produced according to the method of any of embodiments 240-252.
254. The engineered cell of any of embodiments 1-239 and 253, wherein the engineered cell, or progeny or differentiated cells have increased capability to evade NK cell mediated cytotoxicity upon administration to a subject as compared to a cell of the same type that does not comprise the one or more modifications.
255. The engineered cell of any of embodiments 1-239, 253 and 254, wherein the engineered cell, or progeny or differentiated cells derived from the engineered cell undergo reduced cell lysis by mature NK cells upon administration to a subject as compared to a cell of the same type that does not comprise the one or more modifications.
256. The engineered cell of any of embodiments 1-239 and 253-255, wherein the engineered cell, or progeny or differentiated cells derived from the engineered cell induce a reduced immune response upon administration to a subject as compared to a cell of the same type that does not comprise the one or more modifications.
257. The engineered cell of any of embodiments 1-239 and 253-256, wherein the engineered cell, or progeny or differentiated cells derived from the engineered cell induce a reduced systemic inflammatory response upon administration to a subject as compared to a cell of the same type that does not comprise the one or more modifications.
258. The engineered cell of any of embodiments 1-239 and 253-257, wherein the engineered cell, or progeny or differentiated cells derived from the engineered cell induce a reduced local inflammatory response upon administration to a subject as compared to a cell of the same type that does not comprise the one or more modifications.
259. The engineered cell of any of embodiments 1-239 and 253-258, wherein the engineered cell, or progeny or differentiated cells derived from the engineered cell induce reduced complement pathway activation upon administration to a subject as compared to a cell of the same type that does not comprise the one or more modifications.
260. The engineered cell of any of embodiments 1-239 and 253-259, wherein the engineered cell, or progeny or differentiated cells derived from the engineered cell retain the ability to engraft and function upon administration to a subject.
261. The engineered cell of any of embodiments 1-239 and 253-260, wherein the engineered cell, or progeny or differentiated cells derived from the engineered cell has increased ability to engraft and function upon administration to a subject as compared to a cell of the same type that does not comprise the one or more modifications.
262. A population of engineered cells comprising a plurality of the engineered cells of any of embodiments 1-239 and 253-261.
263. The population of engineered cells of embodiment 262, wherein at least about 30% of cells in the population comprise the plurality of the engineered cells.
264. The population of engineered cells of embodiment 262 or embodiment 263, wherein the plurality of the engineered cells are primary cells isolated from more than one donor subject.
265. The population of engineered cells of embodiment 264, wherein each donor subject is healthy or is not suspected of having a disease or condition at the time the donor sample is obtained from the individual donor.
266. A method of producing a composition comprising the engineered cell of any of embodiments 1-239 and 253-261 or the population of engineered cells of any of embodiments 197-200 comprising
   a. obtaining the cell of any of embodiments 225-239;
   b. introducing the one or more modifications of any of embodiments 1-239 into the cell;
   c. selecting the engineered cell or selecting the population of engineered cells from a population of cells based on a level of the one or more of the modifications; and
   d. formulating the composition comprising the selected engineered cell or the selected population of engineered cells.
267. The method of embodiment 266, wherein method comprises selecting the engineered cell or the population of engineered cells based on the level of cell surface expression of the one or more modified molecules in any of embodiments 1-238.
268. The method of embodiment 266 or embodiment 267, wherein the engineered cell or the population of engineered cells are selected based on a level of the one or is more modified molecules having reduced expression in the engineered cell or the population of engineered cells.
269. The method of any of embodiments 266-268, wherein the engineered cell or the population of engineered cells are selected based on a level of the one or more modified molecules having increased expression in the engineered cell or the population of engineered cells.
270. The method of any of embodiments 266-270, wherein the method comprises formulating the composition in a pharmaceutically acceptable additive, carrier, diluent, or excipient.
271. The method of embodiment 270, wherein the pharmaceutically acceptable additive, carrier, diluent, or excipient comprises a pharmaceutically acceptable buffer.
272. The method of embodiment 271, wherein the pharmaceutically acceptable buffer comprises neutral buffer saline or phosphate buffered saline.
273. The method of any of embodiments 266-272, wherein the method comprises formulating the composition with Plasma-Lyte A*, dextrose. dextran, sodium chloride, human serum albumin (HSA), dimethylsulfoxide (DMSO), or a combination thereof.
274. The method of any of embodiments 266-273, wherein the method comprises formulating the composition with a cryoprotectant.
275. The method of any of embodiments 266-274, wherein the method comprises formulating the composition in a serum-free cryopreservation medium comprising a cryoprotectant.
276. The method of embodiment 274 or embodiment 275, wherein the cryoprotectant comprises DMSO.
277. The method of embodiment 274 or embodiment 275, wherein the serum-free cryopreservation medium comprises about 5% to about 10% DMSO (v/v).
278. The method of any of embodiments 275-277, wherein the serum-free cryopreservation medium comprises about 10% DMSO (v/v).
279. The method of any of embodiments 266-278, wherein the method further comprises storing the composition in a container.
280. The method of any of embodiments 266-279, wherein the method further comprises thawing the cel before step (b).
281. The method of any of embodiments 266-280, wherein the method further comprises freezing the engineered cell, the population of engineered cells, or the composition.
282. The method of embodiment 281, wherein the engineered cell or the population of engineered cells are frozen after step (b).
283. The method of embodiment 282, wherein the engineered cell or the population of engineered cells are thawed before step (c).
284. The method of embodiment 281, wherein the engineered cell or the population of engineered cells are frozen after step (c).
285. The method of embodiment 284, wherein the engineered cell or the population of engineered cells are thawed before step (d).
286. The method of embodiment 281, wherein the engineered cell or the population of engineered cells are frozen after step (c).
287. The method of any of embodiments 266-286, wherein the composition is frozen after step (d).
288. A composition comprising the engineered cell of any of embodiments 1-239 and 253-261 or the population of engineered cells of any of embodiments 262-265.
289. A composition produced by the method of any one of embodiments 266-287.
290. The composition of embodiment 288 or embodiment 289, wherein the composition comprises a pharmaceutically acceptable additive, carrier, diluent, or excipient.
291. The composition of any of embodiments 288-290, wherein the composition is sterile.
292. A container comprising the composition of any of embodiments 289-291.
293. The container of embodiment 292, wherein the container is a sterile bag.
294. The container of embodiment 293, wherein the sterile bag is a cryopreservation-compatible bag.
295. A kit comprising the composition of any of embodiments 289-291 or the container of any of embodiments 292-294.
296. The kit of embodiment 295, wherein the kit further comprises instructions for using the engineered cells or the population of engineered cells.

### Embodiments Relating to Uses of Engineered Cells

297. A method of treating a condition or disease in a subject in need thereof comprising administering to the subject an effective amount of the engineered cell of any of embodiments 1-239 and 253-261, the population of engineered cells of any of embodiments 262-265, or the composition of any of embodiments 288-290, optionally wherein the disease or condition is a cellular deficiency.
298. The method of embodiment 297, wherein the condition or disease is selected from the group consisting of diabetes, cancer, vascularization disorders, ocular disease, thyroid disease, skin diseases, and liver diseases.
299. The method of embodiment 297 or 298, wherein the condition or disease is associated with diabetes or is diabetes, optionally wherein the diabetes is Type I diabetes.
300. The method of embodiment 299, wherein the population of engineered cells is a population of islet cells, including beta islet cells.
301. The method of embodiment 300, wherein the islet cells are selected from the group consisting of an islet progenitor cell, an immature islet cell, and a mature islet cell.
302. The method of embodiment 297, wherein the condition or disease is associated with a vascular condition or disease or is a vascular condition or disease.
303. The method of embodiment 302, wherein the engineered cell or the population of engineered cells comprises an endothelial cell.
304. The method of embodiment 297, wherein the condition or disease is associated with autoimmune thyroiditis or is autoimmune thyroiditis.
305. The method of embodiment 304, wherein the engineered cell or the population of engineered cells comprise a thyroid progenitor cell.
306. The method of embodiment 297, wherein the condition or disease is associated with a liver disease or is liver disease.
307. The method of embodiment 306, wherein the liver disease comprises cirrhosis of the liver.
308. The method of embodiment 306 or 307, wherein the engineered cell or the population of engineered cells comprise a hepatocyte or a hepatic progenitor cell.
309. The method of embodiment 297, wherein the condition or disease is associated with a corneal disease or is corneal disease.
310. The method of embodiment 309, wherein the comeal disease is Fuchs dystrophy or congenital hereditary endothelial dystrophy.
311. The method of embodiment 309 or 310, wherein engineered cell or the population of engineered cells comprise a comeal endothelial progenitor cell or a corneal endothelial cell.
312. The method of embodiment 297, wherein the condition or disease is associated with a kidney disease or is kidney disease.
313. The method of embodiment 312, wherein the engineered cell or the population of engineered cells comprise a renal precursor cell or a renal cell.
314. The method of embodiment 297, wherein the condition or disease is associated with a cancer or is cancer.
315. The method of embodiment 314, wherein the cancer is selected from the group consisting of B cell acute lymphoblastic leukemia (B-ALL), diffuse large B-cell lymphoma, liver cancer, pancreatic cancer, breast cancer, ovarian cancer. colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.
316. The method of embodiment 314 or 315, wherein the engineered cell or the population of engineered cells comprise a T cell, an NK cell, or an NKT cell.
317. The method of embodiment 297, wherein the condition or disease is associated with a hematopoietic disease or disorder or is a hematopoietic disease or disorder.
318. The method of embodiment 317, wherein the hematopoietic disease or disorder is myelodysplasia, aplastic anemia, Fanconi anemia, paroxysmal nocturnal hemoglobinuria, Sickle cell disease, Diamond Blackfan anemia, Schachman Diamond disorder. Kostmann's syndrome, chronic granulomatous disease, adrenoleukodystrophy, leukocyte adhesion deficiency, hemophilia, thalassemia, beta-thalassemia, leukaemia such as acute lymphocytic leukemia (ALL), acute myelogenous (myeloid) leukemia (AML). adult lymphoblastic leukaemia, chronic lymphocytic leukemia (CLL), B-cell chronic lymphocytic leukemia (B-CLL), chronic myeloid leukemia (CML). juvenile chronic myelogenous leukemia (CML), and juvenile myelomonocytic leukemia (JMML), severe combined immunodeficiency disease (SCID), X-linked severe combined immunodeficiency, Wiskott-Aldrich syndrome (WAS), adenosine-deaminase (ADA) deficiency, chronic granulomatous disease, Chediak-Higashi syndrome, Hodgkin's lymphoma, non-Hodgkin's lymphoma (NHL) or AIDS.
319. The method of embodiment 297, wherein the condition or disease is associated with leukemia or myeloma or is leukemia or myeloma.
320. The method of embodiment 297, wherein the condition or disease is associated with an autoimmune disease or condition or is an autoimmune disease or condition.
321. The method of embodiment 320, wherein the autoimmune disease or condition is acute disseminated encephalomyelitis, acute hemorrhagic leukoencephalitis, Addison's disease, Agammaglobulinemia, Alopecia areata, amyotrophic lateral sclerosis, ankylosing spondylitis, antiphospholipid syndrome. antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease, Balo concentric sclerosis, Bechets syndrome, Berger's disease, Bickerstaff's encephalitis, Blau syndrome, bullous pemphigoid, cancer. Castleman's disease, celiac disease, chronic inflammatory demyelinating polyneuropathy, chronic recurrent multifocal osteomyelitis, Churg-Strauss syndrome, cicatricial pemphigoid, Cogan syndrome, cold agglutinin disease, complement component 2 deficiency, cranial arteritis, CREST syndrome, Crohn's disease. Cushing's syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, discoid lupus erythematosus, eczema, enthesitis-related arthritis. eosinophilic fasciitis, eosinophilic gastroenteritis. epidermolysis bullosa acquisita, erythema nodosum, essential mixed cryoglobulinemia, Evan's syndrome, firodysplasia ossificans progressiva, fibrosing aveolitis, gastritis, gastrointestinal pemphigoid, giant cell arteritis, glomerulonephritis, goodpasture's syndrome, Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopenic purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD). Lou Gehrig's disease, lupoid hepatitis, lupus erythematosus, Majeed syndrome, Meniere's disease, microscopic polyangiitis. Miller-Fisher syndrome, mixed connective tissue disease, morphea, Mucha-Habermann disease, multiple sclerosis, myasthenia gravis, myositis, neuropyelitis optica, neuromyotonia, ocular cicatricial pemphigoid, opsoclonus myoclonus syndrome, ord thyroiditis, palindromic rheumatism, paraneoplastic cerebellar degeneration, paroxysmal noctumal hemoglobinuria (PNH), Parry Romberg syndrome, Parsonnage-Tumer syndrome, pars planitis, pemphigus, pemphigus vulgaris, permicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia, Takayasu's arteritis, temporal arteritis, Tolosa-Hunt syndrome, transverse myelitis, ulcerative colitis, undifferentiated connective tissue disease, undifferentiated spondylarthropathy, vasculitis, vitiligo or Wegener's granulomatosis.
322. The method of any of embodiments 317-321, wherein engineered cell or the population of engineered cells comprises a hematopoietic stem cell (HSC) or a derivative thereof.
323. The method of embodiment 297, wherein the condition or disease is associated with Parkinson's disease, Huntington disease, multiple sclerosis, a neurodegenerative disease or condition, attention deficit hyperactivity disorder (ADHD), Tourette Syndrome (TS), schizophrenia, psychosis, depression, a neuropsychiatric disorder stroke, or amyotrophic lateral sclerosis (ALS), or wherein the disease or condition is Parkinson's disease, Huntington disease, multiple sclerosis, a neurodegenerative disease or condition, attention deficit hyperactivity disorder (ADHD), Tourette Syndrome (TS), schizophrenia, psychosis, depression, a neuropsychiatric disorder stroke, or amyotrophic lateral sclerosis (ALS).
324. The method of embodiment 323, wherein the engineered cell or the population of engineered cells comprise a neural cell or a glial cell.
325. The method of any of embodiments 297-324, wherein the engineered cell or the population of engineered cells are expanded and cryopreserved prior to administration.
326. The method of any of embodiments 297-325, wherein the method comprises intravenous injection, intramuscular injection, intravascular injection, or transplantation of the engineered cell, the population of engineered cells, or the composition.
327. The method of embodiment 326, wherein transplantation comprises intravascular injection or intramuscular injection.
328. The method of any of embodiments 297-327, wherein the method further comprises administering one or more immunosuppressive agents to the subject.
329. The method of any of embodiments 297-328, wherein the subject has been administered one or more immunosuppressive agents.
330. The method of embodiment 328 or embodiment 329, wherein the one or more immunosuppressive agents are a small molecule or an antibody.
331. The method of any of embodiments 328-330, wherein the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizorbine, 15-deoxyspergualine, 6-mercaptopurine, cyclophosphamide, rapamycin, tacrolimus (FK-506), OKT3, anti-thymocyte globulin, thymopentin (thymosin-a), an immunomodulatory agent, and an immunosuppressive antibody.
332. The method of any of embodiments 328-331, wherein the one or more immunosuppressive agents comprise cyclosporine.
333. The method of any of embodiments 328-331, wherein the one or more immunosuppressive agents comprise mycophenolate rnofetil.
334. The method of any of embodiments 328-331, wherein the one or more immunosuppressive agents comprise a corticosteroid.
335. The method of any of embodiments 328-331, wherein the one or more immunosuppressive agents comprise cyclophosphamide.
336. The method of any of embodiments 328-331, wherein the one or more immunosuppressive agents comprise rapamycin.
337. The method of any of embodiments 328-331, wherein the one or more immunosuppressive agents comprise tacrolimus (FK-506).
338. The method of any of embodiments 328-331, wherein the one or more immunosuppressive agents comprise anti-thymocyte globulin.
339. The method of any of embodiments 328-331, wherein the one or more immunosuppressive agents are one or more immunomodulatory agents.
340. The method of embodiment 339, wherein the one or more immunomodulatory agents are a small molecule or an antibody.
341. The method of embodiment 339 or embodiment 340, wherein the antibody binds to one or more receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.
342. The method of any of embodiments 328-341, wherein the one or more immunosuppressive agents are or have been administered to the subject prior to administration of the engineered cell, the population of engineered cells, or the composition.
343. The method of any of embodiments 328-342, wherein the one or more immunosuppressive agents are or have been administered to the subject at least 1, 2, 3, 4, 5, 6, 7, 8, 9, 10, 11, 12, 13, or 14 days prior to administration of the engineered cell, the population of engineered cells, or the composition.
344. The method of any of embodiments 328-343, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, weeks or more prior to administration of the engineered cell, the population of engineered cells, or the composition.
345. The method of any of embodiments 328-341, wherein the one or more immunosuppressive agents are or have been administered to the subject after administration of the engineered cell, the population of engineered cells, or the composition.
346. The method of any of embodiments 328-341 and 345, wherein the one or more immunosuppressive agents are or have been administered to the subject at least 1, 2, 3, 4, 5, 6, 7, 8, 9, 10, 11, 12, 13, or 14 days after administration of the engineered cell, the population of engineered cells, or the composition.
347. The method of any of embodiments 328-341, 345 and 348, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of the engineered cell, the population of engineered cells, or the composition.
348. The method of any of embodiments 328-341, wherein the one or more immunosuppressive agents are or have been administered to the subject on the same day as the first administration of the engineered cell, the population of engineered cells, or the composition.
349. The method of any of embodiments 328-341, wherein the one or more immunosuppressive agents are or have been administered to the subject after administration of a first and/or second administration of the engineered cell, the population of engineered cells, or the composition.
350. The method of any of embodiments 328-341, wherein the one or more immunosuppressive agents are or have been administered to the subject prior to administration of a first and/or second administration of the engineered cell, the population of engineered cells, or the composition.
351. The method of any of embodiments 328-341, wherein the one or more immunosuppressive agents are or have been administered to the subject at least 1, 2, 3, 4, 5, 6, 7, 8, 9, 10, 11, 12, 13, or 14 days prior to administration of a first and/or second administration of the engineered cell, the population of engineered cells, or the composition.
352. The method of any of embodiments 328-341, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, weeks or more prior to administration of a first and/or second administration of the engineered cell, the population of engineered cells, or the composition.
353. The method of any of embodiments 328-341, wherein the one or more immunosuppressive agents are or have been administered to the subject at least 1, 2, 3, 4, 5, 6, 7, 8, 9, 10, 11, 12, 13, or 14 days after administration of a first and/or second administration of the engineered cell, the population of engineered cells, or the composition.
354. The method of any of embodiments 328-341, wherein the one or more immunosuppressive agents are or have been administered to the subject at least 1 week. 2 weeks, 3 weeks, 4 weeks, 5 weeks, 6 weeks, 7 weeks, 8 weeks, 9 weeks, weeks, or more, after administration of a first and/or second administration of the engineered cell, the population of engineered cells, or the composition.
355. The method of any of embodiments 328-354, wherein the one or more immunosuppressive agents are administered at a lower dosage as compared to the dosage administered to reduce immune rejection of a cell that does not comprise the one or more modifications of the engineered cell or the population of engineered cells.
356. The method of any of embodiments 297-355, wherein the method further comprises activating the safety switch to induce controlled cell death after the administration of the engineered cell, the population of engineered cells, or the composition to the subject.
357. The method of any of embodiments 297-356, wherein the suicide gene or the suicide switch is activated to induce controlled cell death after the administration of the one or more immunosuppressive agents to the subject.
358. The method of any of embodiments 297-356, wherein the suicide gene or the suicide switch is activated to induce controlled cell death prior to the administration of the one or more immunosuppressive agents to the subject.
359. The method of any of embodiments 297-358, wherein the safety switch is activated to induce controlled cell death in the event of cytotoxicity or other negative consequences to the subject.
360. The method of any of embodiments 297-359, wherein the method comprises administering an agent that allows for depletion of the engineered cell, the population of engineered cells, or the composition.
361. The method of embodiment 360, wherein the agent that allows for depletion of the engineered cell is an antibody that recognizes a protein expressed on the cell surface.
362. The method of embodiment 361, wherein the antibody is selected from the group consisting of an antibody that recognizes CCR4, CD16, CD19, CD20, CD30, EGFR, GD2, HER1, HER2, MUC1, PSMA, and RQR8. 363. The method of embodiment 361 or embodiment 362, wherein the antibody is selected from the group consisting of mogamulizumab, AFM13, MOR208, obinutuzumab, ublituximab, ocaratuzumab, rituximab, rituximab-RIIb, tomuzotuximab, R05083945 (GA201), cetuximeb, Hul4.18K322A, Hul4.18-IL2, Hu3F8, dinituximab, c.60C3-Rllc, and biosimilars thereof.
364. The method of any of embodiments 297-361, wherein the method comprises administering an agent that recognizes the one or more tolerogenic factors or the one or more additional tolerogenic factors on the cell surface.
365. The method of any of embodiments 297-364, wherein the method further comprises administering one or more additional therapeutic agents to the subject.
366. The method of any of embodiments 297-364, wherein the subject has been administered one or more additional therapeutic agents.
367. The method of any of embodiments 297-366, wherein the method further comprises monitoring the therapeutic efficacy of the method.
368. The method of any of embodiments 297-367, further comprising monitoring the prophylactic efficacy of the method.
369. The method of embodiment 367 or embodiment 368, wherein the method is repeated until a desired suppression of one or more disease symptoms occurs.

### Embodiments Relating to Lipid Particles. Viral-Like Particles, or Viral Vectors

370. A lipid particle, comprising:
   (a) a retargeted attachment protein comprising (i) a first paramyxovirus envelope attachment protein; and (ii) a first targeting moiety directed to CD8, wherein the first paramyxovirus envelope attachment protein is a variant paramyxovirus envelope attachment protein comprising one or more mutations that reduces the native tropism relative to the wild-type paramyxovirus envelope attachment protein not comprising the one or more mutations;
   (b) at least one paramyxovirus fusion (F) protein; and
   (c) a nucleic acid sequence encoding a chimeric antigen receptor comprising an antibody or antigen binding fragment thereof that specifically binds human Cluster of Differentiation 19 (CD19);
wherein the protein in (a) and (b) are exposed on the outside of the lipid bilayer.
371. A lipid particle, comprising:
   (a) a retargeted attachment protein comprising (i) a first paramyxovirus envelope attachment protein; and (ii) a first targeting moiety directed to CD8, wherein the first paramyxovirus envelope attachment protein is a variant paramyxovirus envelope attachment protein comprising one or more mutations that reduces the native tropism relative to the wild-type paramyxovirus envelope attachment protein not comprising the one or more mutations; and
   (b) at least one paramyxovirus fusion (F) protein; and
   (c) a nucleic acid sequence encoding a chimeric antigen receptor comprising an antibody or antigen binding fragment thereof that specifically binds human Cluster of Differentiation 19 (CD19);
wherein the protein in (a) and (b) are exposed on the outside of the lipid bilayer.
372. The lipid particle of embodiment 370 or 371, further comprising a second paramyxovirus envelope attachment protein that is a variant paramyxovirus envelope attachment protein comprising one or more mutations that reduces the native tropism relative to the wild-type paramyxovirus envelope attachment protein not comprising the one or more mutations.
373. The lipid particle of any of embodiments 370-372, wherein the first and second target molecule are the same target molecule.
374. The lipid particle of embodiment 373, wherein the first and second targeting moiety bind distinct epitopes of the same target molecule.
375. The lipid particle of embodiments 370-374, wherein the first paramyxovirus envelope attachment protein is a variant paramyxovirus envelope attachment protein.
376. The lipid particle of embodiment 375, wherein the variant paramyxovirus envelope attachment protein comprises one or more mutations that reduces native tropism relative to the wild-type paramyxovirus envelope attachment protein not comprising the one or more mutations.
377. The lipid particle of any of embodiments 370-376, wherein the second paramyxovirus envelope attachment protein is a variant paramyxovirus envelope attachment protein.
378. The lipid particle of embodiment 377, wherein the variant paramyxovirus envelope attachment protein comprises one or more mutations that reduces native tropism relative to the wild-type paramyxovirus envelope attachment protein not comprising the one or more mutations.
379. The lipid particle of any of embodiments 370-378, wherein the first targeting moiety is selected from the group consisting of a single domain antibody or a single chain variable fragment (scFv).
380. The lipid particle of embodiment 379, wherein the single domain antibody is a VHH.
381. The lipid particle of any of embodiments 370-380, wherein the first variant paramyxovirus envelope attachment protein and the second variant paramyxovirus envelope attachment protein are the same.
382. The lipid particle of any of embodiments 370-381, wherein the first paramyxovirus envelope attachment protein and the second paramyxovirus envelope attachment protein are different.
383. The lipid particle of any of embodiments 370-382, wherein the first paramyxovirus envelope attachment protein is an envelope attachment protein from a Nipah virus, Hendra virus, or Measles virus, or is a variant or biologically active portion thereof of any of the foregoing.
384. The lipid particle of any of embodiments 370-383, wherein the first paramyxovirus envelope attachment protein is a wild-type paramyxovirus G protein, H protein or HN protein or is a variant or biologically active portion of any of the foregoing.
385. The lipid particle of embodiment 383 or embodiment 384, wherein the first paramyxovirus envelope attachment protein is a wild-type Nipah virus G (NiV-G) protein or is a variant or biologically active portion of a NiV-G.
386. The lipid particle of any of embodiments 383-385, wherein the first paramyxovirus envelope attachment protein is a variant NiV-G that is a variant or a biologically active portion of a wild-type NiV-G.
387. The lipid particle of any of embodiments 370-388, wherein the second paramyxovirus envelope attachment protein is an envelope attachment protein from a Nipah virus, Hendra virus, or Measles virus, or is a variant or biologically active portion of any of the foregoing.
388. The lipid particle of any of embodiments 370-387, wherein the second paramyxovirus envelope attachment protein is a wild-type paramyxovirus G protein, H protein or HN protein or is a variant or biologically active portion of any of the foregoing.
389. The lipid particle of embodiment 387 or embodiment 388, wherein the second paramyxovirus envelope attachment protein is a wild-type Nipah virus G (NiV-G) protein or is a variant or a biologically active portion of a NIV-G.
390. The lipid particle of any of embodiments 370-388, wherein the second paramyxovirus envelope attachment protein is a variant NiV-G that is a variant or a biologically active portion of a wild-type NiV-G.
391. The lipid particle of any of embodiments 370-390, wherein the second paramyxovirus envelope attachment protein is a variant paramyxovirus envelope glycoprotein from a Nipah virus, Hendra virus, or Measles virus or a biologically active portion thereof.
392. The lipid particle of any of embodiments 370-391, wherein the second paramyxovirus envelope attachment protein is a variant of a wild-type paramyxovirus G protein, H protein or HN protein or a biologically active portion thereof.
393. The lipid particle of any of embodiments 370-392, wherein the third paramyxovirus envelope attachment protein is an envelope attachment protein from a Nipah virus, Hendra virus, or Measles virus, or is a variant or biologically active portion of any of the foregoing.
394. The lipid particle of any of embodiments 370-393, wherein the third paramyxovirus envelope attachment protein is a wild-type paramyxovirus G protein, H protein or HN protein or is a variant or biologically active portion of any of the foregoing.
395. The lipid particle of embodiment 393 or embodiment 394, wherein the third paramyxovirus envelope attachment protein is a wild-type Nipah virus G (NiV-G) protein or is a variant or a biologically active portion of a NiV-G.
396. The lipid particle of any of embodiments 370-395, wherein the third paramyxovirus envelope attachment protein is a variant NiV-G that is a variant or a biologically active portion of a wild-type NiV-G.
397. The lipid particle of any one of embodiments 390, 391. 395, or 396, wherein the variant is a variant NiV-G that is a variant of a wild-type Nipah virus G (NiV-G) protein or a biologically active portion thereof.
398. The lipid particle of any of embodiments 370-397, wherein the at least one paramyxovirus fusion (F) protein is an F protein from a henipavirus or is a biologically active portion thereof or variant thereof.
399. The lipid particle of embodiment 398, wherein the henipavirus is a Hendra virus.
400. The lipid particle of embodiment 399, wherein the henipavirus is a Nipah virus.
401. The lipid particle of any of embodiments 370400, wherein the paramyxovirus F protein is a wild-type NiV-F protein or a variant or a biologically active portion thereof.
402. The lipid particle of any of embodiments 370-401, wherein the paramyxovirus F protein is a variant NiV-F that is a variant or a biologically active portion of a wild-type NiV-F protein.
403. The lipid particle of any of embodiments 370-402, wherein the paramyxovirus F protein is an F0 precursor or is a proteolytically cleaved form thereof comprising F1 and F2 subunits.
404. The lipid particle of embodiment 403, wherein the proteolytically cleaved form is a cathepsin L cleavage product.
405. The lipid particle of any of embodiments 370-404, wherein the first targeting moiety and the first paramyxovirus envelope attachment protein or biologically active portion thereof is attached via a linker.
406. The lipid particle of any of embodiments 370-409, wherein the second targeting moiety and the second paramyxovirus envelope attachment protein or biologically active portion thereof is attached via a linker.
407. The lipid particle of embodiment 405 or embodiment 406, wherein the linker is a peptide linker.
408. The lipid particle of embodiment 407, wherein the peptide linker is 2 to 65 amino acids in length.
409. The lipid particle of embodiment 407 or embodiment 408, wherein the peptide linker is a flexible linker that comprises GS, GGS, GGGGS, GGGGGS or combinations thereof.
410. The lipid particle of any of embodiments 407-409, wherein the peptide linker is selected from: (GGS)n, wherein n is 1 to 10; (GGGGS)n, wherein n is 1 to 10; or (GGGGGS)n, wherein n is 1 to 6.
411. The lipid particle of any of embodiments 370-410, wherein the lipid particle further comprises one or more additional paramyxovirus envelope attachment glycoproteins embedded in the lipid bilayer.
412. The lipid particle of embodiment 411, wherein the one or more additional paramyxovirus envelope attachment glycoproteins is a retargeted attachment protein comprising a paramyxovirus envelope attachment protein and a further targeting moiety.
413. The lipid particle of any of embodiments 412, wherein the at least one paramyxovirus fusion (F) protein exhibits fusogenic activity with a target cell upon binding of at least one paramyxovirus envelope attachment protein o to the target molecule on the target cell.
414. The lipid particle of any of embodiments 370-413, wherein the lipid particle comprises a viral nucleic acid.
415. The lipid particle of embodiment 414, wherein the viral nucleic acid comprises one or more of (e.g., all of) the following nucleic acid sequences: 5′ LTR (e.g.. comprising U5 and lacking a functional U3 domain), Psi packaging element (Psi), Central polypurine tract (cPPT)central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).
416. The lipid particle of any of embodiments 370-415, wherein the lipid particle is a viral vector.
417. The lipid particle of any of embodiments 370-416, that is a retroviral vector.
418. The lipid particle of any of embodiments 370-417, that is a lentiviral vector.
419. The lipid particle of any of embodiments 370-418, wherein the lipid particle is devoid of viral genomic DNA.
420. The lipid particle of any of embodiments 370-419, that is a viral-like particle.
421. The lipid particle of any of embodiments 370-420, that is a retroviral-like particle.
422. The lipid particle of any of embodiments 370-421, that is a lentiviral-like particle.
423. The lipid particle of embodiment 422, wherein the lentiviral-like particle is a HIV-like particle or is an MLV-like particle.
424. The lipid particle of any of embodiments 370-423, wherein the lipid particle is produced as a preparation with increased titer compared to a reference lipid particle preparation that is similarly produced but with only the first retargeted attachment protein.
425. A producer cell comprising (a) a nucleic acid encoding a retargeted attachment protein comprising a first paramyxovirus envelope attachment protein; and (i) a first targeting moiety directed to a first target molecule expressed on the surface of a target cell, (b) a nucleic acid encoding a second paramyxovirus attachment protein, (c) a nucleic acid encoding at least one paramyxovirus F protein, and (d) a nucleic acid sequence encoding a chimeric antigen receptor comprising an antibody or antigen binding fragment thereof that specifically binds human Cluster of Differentiation 19 (CD19); wherein the second paramyxovirus attachment protein is: (1) a second retargeted attachment protein comprising (i) a second paramyxovirus envelope attachment protein; and (ii) a second targeting moiety directed to a second target molecule expressed on the surface of the target cell; or (2) a variant paramyxovirus envelope attachment protein comprising one or more mutations to reduce native tropism relative to the wild-type paramyxovirus envelope attachment protein or the biologically active portion thereof not comprising the one or more mutations, wherein targeting one or both of the first target molecule and the second target molecule does not modulate or induce a signal in the target cell.
426. A producer cell comprising: (a) a nucleic acid encoding a first retargeted attachment protein comprising (i) a first paramyxovirus envelope attachment protein; and (ii) a first targeting moiety directed to a first target molecule expressed on the surface of a target cell, and (b) a nucleic acid encoding a second retargeted attachment protein comprising (i) a second paramyxovirus envelope attachment protein; and (ii) a second targeting moiety directed to a second target molecule expressed on the surface of a target cell, (c) a nucleic acid encoding at least one paramyxovirus (F) protein, and (d) a nucleic acid sequence encoding a chimeric antigen receptor comprising an antibody or antigen binding fragment thereof that specifically binds human Cluster of Differentiation 19 (CD19); wherein each of the first targeting moiety and the second targeting moiety are independently selected from the group consisting of an antibody or antigen-binding fragment, a DARPin, and a targeting peptide.
427. A producer cell comprising (a) a nucleic acid encoding a retargeted is attachment protein comprising (i) a first paramyxovirus envelope attachment protein; and (ii) a first targeting moiety directed to a first target molecule expressed on the surface of a target cell, (b) a nucleic acid encoding a second paramyxovirus envelope attachment protein that is a variant paramyxovirus envelope attachment protein comprising one or more mutations to reduce the native tropism relative to the wild-type paramyxovirus envelope attachment protein or the biologically active portion thereof not comprising the one or more mutations: (c) a nucleic acid encoding at least one paramyxovirus fusion (F) protein; and (d) a nucleic acid sequence encoding a chimeric antigen receptor comprising an antibody or antigen binding fragment thereof that specifically binds human Cluster of Differentiation 19 (CD19);.
428. A producer cell comprising (a) a nucleic acid encoding a first retargeted attachment protein comprising (i) a first paramyxovirus envelope attachment protein; and (ii) a first targeting moiety directed to a first target molecule expressed on the surface of a target cell, and (b) a nucleic acid encoding a second retargeted attachment protein comprising (i) a second paramyxovirus envelope attachment protein; and (ii) a second targeting moiety directed to a second target molecule expressed on the surface of a target cell, (c) a nucleic acid encoding a second paramyxovirus envelope attachment protein that is a variant paramyxovirus envelope attachment protein comprising one or more mutations to reduce the native tropism relative to the wild-type paramyxovirus envelope attachment protein or the biologically active portion thereof not comprising the one or more mutations; and (d) a nucleic acid encoding at least one paramyxovirus (F) protein, wherein each of the first targeting moiety and the second targeting moiety are independently selected from the group consisting of an antibody or antigen-binding fragment, a DARPin, and a targeting peptide.
429. The producer cell of any of embodiments 425-428, wherein the cell further comprises a viral nucleic acid(s).
430. The producer cell of embodiment 429, wherein the viral nucleic acid(s) are lentiviral nucleic acids.
431. The producer cell of any of embodiments 425-430, wherein the cell is a mammalian cell.
432. The producer cell of any of embodiments 425-431, wherein the producer cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells. BSC 40 cells, BMT 10 cells, VERO cells, W138 cells. MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.
433. The producer cell of any of embodiments 425-432, wherein the producer cell comprises 293T cells.
434. The producer cell of any of embodiments 425-433, wherein the viral nucleic acid(s) lacks one or more genes involved in viral replication.
435. The producer cell of any of embodiments 425-434, wherein the viral nucleic acid comprises a nucleic add encoding a viral packaging protein selected from one or more of Gag, Pol, Rev and Tat.
438. The producer cell of any of embodiments 425-435, wherein the viral nucleic acid comprises: one or more of (e.g., all of) the following nucleic acid sequences: 5′ LTR (e.g., comprising US and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).
437. The producer cell of any of embodiments 425-436, wherein the target molecule is selected from the group consisting of CD3, CD4, CD7 CD8, ASCT2, CD105, CD110, CD146. CD164, CD34, CD46, CD49f, CD90. EPCR, and ITGA3.
438. A method of transducing a cell comprising contacting a cell with a lipid particle of any of embodiments 370-424 or a composition comprising the lipid particle of any embodiments 370-424.
439. A method of delivering an exogenous agent into a target cell (e.g., a CD8* T cell), the method comprising contacting a lipid particle of any of embodiments 370-424 or a composition comprising the lipid particle of any embodiments 370-424 with a target cell.
440. The method of any of embodiment 442 or 443, wherein the contacting is in vitro or ex vivo.
441. The method of any of embodiment 442 or 443, wherein the contacting is in vivo in a subject.
442. A method of delivering an exogenous agent to a cell in a subject, the method comprising administering to the subject a lipid particle of any of embodiments 370-424 or a composition comprising the lipid particle of any embodiments 370-424.
443. The method of embodiment 446, wherein the exogenous agent is or encodes a therapeutic agent for treating a disease or condition in the subject.
445. A method of treatment, the method comprising administering to a subject a lipid particle of any of embodiments 370-424 or a composition comprising the lipid particle of any embodiments 370-424.
446. The method of any of embodiments 441-449, wherein the exogenous agent is a chimeric antigen receptor for targeting an antigen associated with a disease or condition in the subject, where the disease is cancer or an autoimmune disease.
447. The method of embodiment 446, wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
448. The method of any of embodiments 441-447, wherein the exogenous agent is for use in gene therapy to correct a genetic deficiency or replaces a deficient or missing gene in the subject.
449. The method of any of embodiments 441-448, wherein the subject is a human subject.

### Embodiments Relating to Using Engineered T Cells for Treating a Subject 450

A method of treating cancer in a patient that is suspected of having cancer or has been diagnosed with cancer comprising administering a population of engineered T cells to the patient, wherein the engineered T cells comprise reduced expression of one or more MHC class I and/or class It HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, a hinge domain, a transmembrane domain, a co-stimulatory domain, and an intracellular signaling domain.

451. A method of treating cancer in a patient that is suspected of having cancer or has been diagnosed with cancer comprising administering a population of engineered T cells to the patient, wherein the engineered T cells comprise reduced expression of beta-2-microglobulin (12M) relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
452. A method of treating cancer in a patient that is suspected of having cancer or has been diagnosed with cancer comprising administering a population of engineered T cells to the patient, wherein the engineered T cels comprise reduced expression of B2M and MHC class II transactivator (CIITA) relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
453. A method of treating cancer in a patient that is suspected of having cancer or has been diagnosed with cancer comprising administering a population of engineered T cells to the patient, wherein the engineered T cells comprise reduced expression of B2M and MHC class II transactivator (CIITA) relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
454. A method of treating cancer in a patient that is suspected of having cancer or has been diagnosed with cancer comprising administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
455. A method of treating cancer in a patient that is suspected of having cancer or has been diagnosed with cancer comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
456. A method of treating cancer in a patient that is suspected of having cancer or has been diagnosed with cancer comprising administering a population of engineered T cells to the patient, wherein the engineered T cells comprise reduced expression of one or more MHC class I and/or class II HLA molecules relative to an unaltered or unmodified wild-type or control cell, reduced expression of TCR-alpha (TRAC) and/or TCR-beta (TRB) relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, a hinge domain, a transmembrane domain, a co-stimulatory domain, and an intracellular signaling domain.
457. A method of treating cancer in a patient that is suspected of having cancer or has been diagnosed with cancer comprising administering a population of engineered T cells to the patient, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
458. A method of treating cancer in a patient that is suspected of having cancer or has been diagnosed with cancer comprising administering a population of engineered T cells to the patient, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
459. A method of treating cancer in a patient that is suspected of having cancer or has been diagnosed with cancer comprising administering a population of engineered T cells to the patient, wherein the engineered T cells comprise reduced expression of B2M relative to an unaltered or unmodified wild-type or control cell. reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
460. A method of treating cancer in a patient that is suspected of having cancer or has been diagnosed with cancer comprising administering a population of engineered T cells to the patient, wherein the engineered T cels comprise reduced expression of 82M and CIITA relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
461. A method of treating cancer in a patient that is suspected of having cancer or has been diagnosed with cancer comprising administering a population of engineered T cells to the patient, wherein the engineered T cels comprise reduced expression of one or more MHC class I and/or class II HLA molecules relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
462. A method of treating cancer in a patient that is suspected of having cancer or has been diagnosed with cancer comprising evaluating the patient for and/or diagnosing the patient with EBV infection and optionally cancer, and administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, a hinge domain, a transmembrane domain, a co-stimulatory domain, and an intracellular signaling domain.
463. The method according to any one of embodiments 450-462, wherein the one or more CARs comprise a CD8a hinge domain, a CD28 hinge domain, or an IgG4 hinge domain.
464. The method according to embodiment 463, wherein the one or more CARs comprise a CD8a hinge domain having the amino acid sequence of SEQ ID NO: 34.
465. The method according to embodiment 463, wherein the one or more CARs comprise a CD28 hinge domain having the amino acid sequence of SEQ ID NO: 35 or 36.
466. The method according to embodiment 463, wherein the one or more CARs comprise a IgG4 hinge domain having the amino acid sequence of SEQ ID NO: 37, 38 or 39.
467. The method according to any one of embodiments 450-466, wherein the one or more CARs comprise a CD8a transmembrane domain or a CD28 transmembrane domain.
488. The method according to embodiment 467, wherein the one or more CARs comprise a CD8a transmembrane domain having the amino acid sequence of SEQ ID NO: 49.
469. The method according to embodiment 488, wherein the one or more CARs comprise a CD28 transmembrane domain having the amino acid sequence of SEQ ID NO: 41 or 42.
470. The method according to any one of embodiments 450-469, wherein the one or more CARs comprise a 4-18B costimulatory domain, a CD28 costimulatory domain, or a CD3(signaling domain.
471. The method according to embodiment 470, wherein the one or more CARs comprise a 4-1BB costimulatory domain having the amino acid sequence of SEQ ID NO: 43.
472. The method according to embodiment 470, wherein the one or more CARs comprise a CD28 costimulatory domain having the amino acid sequence of SEQ ID NO: 44.
473. The method according to embodiment 470, wherein the one or more CARs comprise a CD34 signaling domain having the amino acid sequence of SEQ ID NO: 46 or 47.
474. The method according to any one of embodiments 450-473, wherein the one or more CARs comprise an extracellular ligand-binding domain comprising an scFv sequence of any one of SEQ ID NOs: 25, 26. 27, 228.229, or 230, or wherein the CARs have an scFv sequence comprising the heavy and light chain sequences of any one of SEQ ID NOs: 19-21 or 22-24.
475. The method according to any one of embodiments 450-474, wherein the one or more CARs have a sequence of any one of SEQ ID NOs: 232, 234, 236, 238, 240, or 242.
476. A method of treating multiple sclerosis in a patient that is suspected of having multiple sclerosis or has been diagnosed with multiple sclerosis comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, a hinge domain, a transmembrane domain, a co-stimulatory domain, and an intracellular signaling domain.
477. A method of treating multiple sclerosis in a patient that is suspected of having multiple sclerosis or has been diagnosed with multiple sclerosis comprising administering a population of engineered T cells to the patient, wherein the engineered T cells comprise reduced expression of beta-2-microglobulin (B2M) relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
478. A method of treating multiple sclerosis in a patient that is suspected of having multiple sclerosis or has been diagnosed with multiple sclerosis comprising administering a population of engineered T cells to the patient, wherein the engineered T cells comprise reduced expression of 82M and MHC class II transactivator (CIITA) relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
479. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and MHC class II transactivator (CIITA) relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
480. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
481. A method of treating multiple sclerosis in a patient that is suspected of having multiple sclerosis or has been diagnosed with multiple sclerosis comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
482. A method of treating multiple sclerosis in a patient that is suspected of having multiple sclerosis or has been diagnosed with multiple sclerosis comprising administering a population of engineered T cells to the patient, wherein the engineered T cells comprise reduced expression of one or more MHC class I and/or class II HLA molecules relative to an unaltered or unmodified wild-type or control cell, reduced expression of TCR-alpha (TRAC) and/or TCR-beta (TRB) relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, a hinge domain, a transmembrane domain, a co-stimulatory domain, and an intracellular signaling domain.
483. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
484. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
485. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
486. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
487. A method of treating multiple sclerosis in a patient that is suspected of having multiple sclerosis or has been diagnosed with multiple sclerosis comprising administering a population of engineered T cells to the patient, wherein the engineered T cells comprise reduced expression of one or more MHC class I and/or class II HLA molecules relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
488. A method of treating multiple sclerosis in a patient that is suspected of having multiple sclerosis or has been diagnosed with multiple sclerosis comprising evaluating the patient for and/or diagnosing the patient with EBV infection and optionally multiple sclerosis, and administering a population of engineered T cells to the patient, wherein the engineered T cells comprise reduced expression of one or more MHC class I and/or class It HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, a hinge domain, a transmembrane domain, a co-stimulatory domain, and an intracellular signaling domain.
489. The method according to any one of embodiments 476-488, wherein the one or more CARs comprise a CD8a hinge domain, a CD28 hinge domain, or an IgG4 hinge domain.
490. The method according to embodiment 489, wherein the one or more CARs comprise a CD8a hinge domain having the amino acid sequence of SEQ ID NO: 34.
491. The method according to embodiment 489, wherein the one or more CARs comprise a CD28 hinge domain having the amino acid sequence of SEQ ID NO: 35 or 36.
492. The method according to embodiment 489, wherein the one or more CARs comprise a IgG4 hinge domain having the amino acid sequence of SEQ ID NO: 37, 38 or 39.
493. The method according to any one of embodiments 478-492, wherein the one or more CARs comprise a CD8a transmembrane domain or a CD28 transmembrane domain.
494. The method according to embodiment 493, wherein the one or more CARs comprise a CD8a transmembrane domain having the amino acid sequence of SEQ ID NO: 40.
495. The method according to embodiment 494, wherein the one or more CARs comprise a CD28 transmembrane domain having the amino acid sequence of SEQ ID NO: 41 or 42.
496. The method according to any one of embodiments 476-495, wherein the one or more CARs comprise a 4-18B costimulatory domain, a CD28 costimulatory domain, or a CD3(signaling domain.
497. The method according to embodiment 496, wherein the one or more CARs comprise a 4-1BB costimulatory domain having the amino acd sequence of SEQ ID NO: 43.
498. The method according to embodiment 496, wherein the one or more CARs comprise a CD28 costimulatory domain having the amino acid sequence of SEQ ID NO: 44 or 45.
499. The method according to embodiment 496, wherein the one or more CARs comprise a CD34 signaling domain having the amino acid sequence of SEQ ID NO: 46 or 47.
500. The method according to any one of embodiments 476-499, wherein the one or more CARs comprise an extracellular ligand-binding domain comprising an scFv sequence of any one of SEQ ID NOs: 25, 26, 27, 228, 229, or 230, or wherein the CARs have an scFv sequence comprising the heavy and light chain sequences of any one of SEQ ID NOs: 19-21 or 22-24.
501. The method according to any one of embodiments 476-500, wherein the one or more CARs have a sequence of any one of SEQ ID NOs: 232, 234, 236, 238, 240, or 242.
502. A method of treating multiple sclerosis in a patient that is suspected of having multiple sclerosis or has been diagnosed with multiple sclerosis and administering a population of engineered T cells to the patient, wherein the engineered T cells comprise reduced expression of B2M, CIITA, and TRAC and/or TRB, relative to an unaltered or unmodified wild-type or control ceil, a first exogenous polynucleotide encoding CD47, and a second exogenous polynucleotide encoding one or more CARs, wherein the one or more CARs comprise a CD19 CAR having the HCDR sequences of Table 7 or Table 9 and LCDR sequences of Table 8 or Table 10.
503. A method of treating multiple sclerosis in a patient that is suspected of having multiple sclerosis or has been diagnosed with multiple sclerosis comprising administering a population of engineered T cells to the patient, wherein the engineered T cells comprise an exogenous polynucleotide encoding one or more CARs wherein the one or more CARs have a sequence of any one of SEQ ID NOs: 232, 234, 236. 238, 240, or 242, or wherein the one or more CARs have an scFv sequence of any one of SEQ ID NOs: 25, 26, 27, 228. 229, or 230, or wherein the CARs have an scFv sequence comprising the heavy and light chain sequences of any one of SEQ ID NOs: 19-21 or 22-24.
504. The method of any one of embodiments 450-503, further comprising evaluating the patient for and/or diagnosing the patient with EBV infection and optionally multiple sclerosis prior to administering the population of engineered T cells to the patient.
505. The method of embodiment 504, wherein the diagnosis comprises evaluating the patient for EBV infection.
506. The method of embodiment 504 or 505, wherein the diagnosis comprises evaluating the patient for multiple sclerosis.
507. The method according to any one of embodiments 476-506, wherein the treatment prevents multiple sclerosis.
508. The method according to any one of embodiments 476-507, wherein the treatment treats multiple sclerosis.
509. The method according to any one of embodiments 476-508, wherein the patient with the EBV infection has been diagnosed with multiple sclerosis.
510. The method according to anyone of embodiments 476-509, wherein the multiple sclerosis is relapsing-remitting multiple sclerosis, progressive relapsing multiple sclerosis, primary progressive multiple sclerosis, or secondary progressive multiple sclerosis.
511. The method according to anyone of embodiments 476-510, wherein the patient undergoes remission of multiple sclerosis following administration of the engineered T cells.
512. The method according to any one of embodiments 476-511. wherein the patient with the EBV infection is undergoing treatment for the EBV infection.
513. The method according to any one of embodiments 476-512, wherein the patient with the EBV infection has an active EBV infection.
514. The method according to any one of embodiments 476-513, wherein the patient with the EBV infection has an inactive EBV infection.
515. The method according to any one of embodiments 476-514, wherein the patient undergoes a reduced EBV infection following administration of the engineered T cells, optionally wherein the reduced EBV infection is characterized by reduced viral load.
516. The method according to anyone of embodiments 476-515, wherein the treatment prevents an EBV infection change from an inactive to an active EBV infection.
517. The method according to anyone of embodiments 476-516, wherein the method results in B cell depletion.
518. The method according to anyone of embodiments 476-517, wherein the engineered T cells comprise one or more of a CD19-specific CAR, a CD20-specific CAR, a CD22-specific CAR, a BCMA-specific CAR, a GPRCSD-specific CAR, a CD38-specific CAR, a CD70-specific CAR, a CD79b-specifc CAR, and an EBV antigen-specific CAR.
519. A method of treating an autoimmune disease in a patient that is suspected of having the autoimmune disease or has been diagnosed with the autoimmune disease comprising administering a population of engineered T cells to the patient, wherein the engineered T cells comprise an exogenous polynucleotide encoding one or more CARs, wherein the one or more CARs comprise an extracellular ligand-binding domain having specificity for CD19, a hinge domain, a transmembrane domain. a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.
520. A method of treating an autoimmune disease in a patient that is suspected of having the autoimmune disease or has been diagnosed with the autoimmune disease comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
521. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
522. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for C019, a hinge domain, a transmembrane domain, a co-stimulatory domain, and an intracellular signaling domain, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease. Myasthenia Gravis, Stiff-Person syndrome, or a pulmonary condition.
523. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
524. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for a human CD19 antigen, a hinge domain, a transmembrane domain, a co-stimulatory domain, and an intracellular signaling domain, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
525. The method according to any one of embodiments 519-524, wherein the one or more CARs comprise a CD8a hinge domain, a CD28 hinge domain, or an IgG4 hinge domain.
526. The method according to embodiment 525, wherein the one or more CARs comprise a CD8a hinge domain having the amino acid sequence of SEQ ID NO: 34.
527. The method according to embodiment 525, wherein the one or more CARs comprise a CD28 hinge domain having the amino acid sequence of SEQ ID NO: 35 or 36.
528. The method according to embodiment 525, wherein the one or more CARs comprise a IgG4 hinge domain having the amino acid sequence of SEQ ID NO: 37, 38, or 39.
529. The method according to any one of embodiments 519-528, wherein the one or more CARs comprise a CD8a transmembrane domain or a CD28 transmembrane domain.
530. The method according to embodiment 529, wherein the one or more CARs comprise a CD8a transmembrane domain having the amino acid sequence of SEQ ID NO: 40.
531. The method according to embodiment 529, wherein the one or more CARs comprise a CD28 transmembrane domain having the amino acid sequence of SEQ ID NO: 41 or 42.
532. The method according to any one of embodiments 519-528, wherein the one or more CARs comprise a 4-1BB costimulatory domain, a CD28 costimulatory domain, or a CD34 signaling domain.
533. The method according to embodiment 532, wherein the one or more CARs comprise a 4-1BB costimulatory domain having the amino acid sequence of SEQ ID NO: 43.
534. The method according to embodiment 532, wherein the one or more CARs comprise a CD28 costimulatory domain having the amino acid sequence of SEQ ID NO: 44 or 45.
535. The method according to embodiment 532, wherein the one or more CARs comprise a CD3(signaling domain having the amino acid sequence of SEQ ID NO: 46 or 47.
536. The method according to any one of embodiments 519-535, wherein the one or more CARs comprise an extracellular ligand-binding domain comprising an scFv sequence of any one of SEQ ID NOs: 25, 26, 27, 228, 229, or 230, or wherein the CARs have an scFv sequence comprising the heavy and light chain sequences of any one of SEQ ID NOs: 19-21 or 22-24.
537. The method according to any one of embodiments 519-537, wherein the one or more CARs have a sequence of any one of SEQ ID NOs: 232, 234, 236, 238, 240, or 242.
538. A method of treating an autoimmune disease in a patient that is suspected of having the autoimmune disease or has been diagnosed with the autoimmune disease comprising: administering a population of engineered T cells to the patient, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the HCDR sequences of Table 7 or Table 9 and LCDR sequences of Table 8 or Table 10, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
539. The method according to embodiment 538, further comprising evaluating the patient for and/or diagnosing the patient with the autoimmune disease.
540. A method of treating an autoimmune disease in a patient that is suspected of having the autoimmune disease or has been diagnosed with the autoimmune disease comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 232, 234, 236, 238, 240, or 242, or wherein the one or more CARs have an scFv sequence of any one of SEQ ID NOs: 25, 26. 27, 228, 229, or 230.
541. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M relative to an unaltered or unmodified wild-type or control cel, a first exogenous polynucleotide encoding a tolerogenic factor, and a second exogenous polynucleotide encoding one or more CARs, wherein the one or more CARs have a sequence of any one of SEQ ID NOs: 232, 234, 236, 238, 240, or 242, or wherein the one or more CARs have an scFv sequence of any one of SEQ ID NOs: 25, 26, 27, 228, 229, or 230.
542. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 232, 234, 236, 238, 240, or 242, or wherein the one or more CARs have an scFv sequence of any one of SEQ ID NOs: 25, 26, 27, 228, 229, or 230.
543. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 232, 234, 236, 238, 240, or 242, or wherein the one or more CARs have an scFv sequence of any one of SEQ ID NOs: 25, 26, 27, 228, 229, or 230.
544. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 232, 234, 236, 238, 240, or 242, or wherein the one or more CARs have an scFv sequence of any one of SEQ ID NOs: 25, 26, 27, 228, 229, or 230.
545. A method of treating an autoimmune disease in a patient that is suspected of having the autoimmune disease or has been diagnosed with the autoimmune disease comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs wherein the one or more CARs have a sequence of any one of SEQ ID NOs: 232, 234, 236, 238, 240, or 242, or wherein the one or more CARs have an scFv sequence of any one of SEQ ID NOs: 25, 26, 27, 228, 229, or 230.
546. The method of any one of embodiments 538-545, further comprising evaluating the patient for and/or diagnosing the patient with the autoimmune disease prior to administering the population of engineered T cells to the patient.
547. The method of any one of embodiments 538-546, wherein the autoimmune disease is selected from the group consisting of lupus, systemic lupus erythematosus, multiple sclerosis, systemic sclerosis, vasculitis, Crohn's disease, Myasthenia Gravis, Stiff-Person syndrome, and a pulmonary condition.
548. The method of any one of embodiments 538-547, wherein the patient is suspected of having multiple sclerosis or has been diagnosed with multiple sclerosis.
549. The method of any one of embodiments 450-548, further comprising administering a second, third, fourth, fifth, or sixth dose of the engineered T cells to the patient.
550. The method of embodiment 549, wherein the CAR is the same in one or more of the first, second, third, fourth, fifth, and/or sixth dose of engineered T cells.
551. The method of embodiment 549, wherein the CAR is different in one or more of the first, second, third, fourth, fifth, and/or sixth dose of engineered T cells.
552. The method of any one of embodiments 450-551, wherein the CAR has an scFv sequence of any one of SEQ ID NOs: 25, 26, 27, 228, 229, or 230, or wherein the CARs have an scFv sequence comprising the heavy and light chain sequences of any one of SEQ ID NOs: 19-21 or 22-24.
553. The method of any one of embodiments 450-552, wherein the CAR has a sequence of any one of SEQ ID NOs: 232, 234, 236, 238, 240, or 242.
554. The method of any one of embodiments 450-553, wherein the engineered T cells comprise a CD19-specific CAR and a CD20-specific CAR.
555. The method of embodiment 554, wherein the CD19-specific CAR and the CD20-specific CAR are encoded by a single bicistronic polynucleotide.
556. The method of embodiment 554, wherein the CD19-specific CAR and the CD20-specific CAR are encoded by a single bispecific CAR.
557. The method of embodiment 554, wherein the CD19-specific CAR and the CD20-specific CAR are encoded by two separate polynucleotides.
558. The method of anyone of embodiments 554-557, wherein the CD19 CAR T cells and CD20 CAR T cells are administered concomitantly.
559. The method of anyone of embodiments 554-558, wherein the CD19 CAR+ T cells and CD20 CAR+ T cells are administered sequentially.
560. The method of embodiment 559, wherein the CD19 CAR+ T cells are administered prior to administration of the CD20 CAR+ T cells.
561. The method of embodiment 559, wherein the CD20 CAR+ T cells are administered prior to administration of the CD19 CAR+ T cells.
562. The method of any one of embodiments 554-561, wherein the number of cells administered as a therapeutically effective amount of the CD19 and/or CD20 CAR T cells is greater than the number of cells administered as a therapeutically effective amount of CD19 CAR T cells or CD20 CAR T cells alone.
563. The method of any one of embodiments 554-562, wherein the number of cells administered to as a therapeutically effective amount of the CD19 and/or CD20 CAR T cells is less than the number of cells administered as a therapeutically effective amount of C019 CAR T cells or CD20 CAR T cells alone.
564. The method of any one of embodiments 450-563, wherein the engineered T cells comprise an EBV antigen-specific CAR and a CD19-specific CAR.
565. The method of embodiment 564, wherein the EBV antigen-specific CAR and the CD19-specific CAR are encoded by a single bicistronic polynucleotide.
566. The method of embodiment 565, wherein the EBV antigen-specific CAR and the CD19-specific CAR are encoded by a single bispecific CAR.
567. The method of embodiment 565, wherein the EBV antigen-specific CAR and the CD19-specific CAR are encoded by two separate polynucleotides.
568. The method of any one of embodiments 565-567, wherein the EBV antigen CAR T cells and CD19 CAR T cells are administered concomitantly.
569. The method of any one of embodiments 565-567, wherein the EBV antigen CAR+ T cells and CD19 CAR+ T cells are administered sequentially.
570. The method of embodiment 569, wherein the EBV antigen CAR+ T cells are administered prior to administration of the CD19 CAR+ T cells.
571. The method of embodiment 569, wherein the CD19 CAR+ T cells are administered prior to administration of the EBV antigen CAR+ T cells.
572. The method of any one of embodiments 564-571, wherein the number of cells administered as a therapeutically effective amount of the EBV antigen and/or CD19 CAR T cells is greater than the number of cells administered as a therapeutically effective amount of EBV antigen CAR T cells or CD19 CAR T cells alone.
573. The method of any one of embodiments 564-571, wherein the number of cells administered to as a therapeutically effective amount of the EBV antigen and/or CD19 CAR T cells is less than the number of cells administered as a therapeutically effective amount of EBV antigen CAR T cells or CD19 CAR T cells alone.
574. The method of any one of embodiments 450-573, wherein the engineered T cells are primary T cells, are propagated from a primary T cell or a progeny thereof, or are derived from a T cell differentiated from an iPSC or a progeny thereof.
575. The method of any one of embodiments 450-574, wherein the engineered T cells are differentiated cells derived from an induced pluripotent stem cell or a progeny thereof.
576. The method of embodiment 258, wherein the differentiated cells are a T cells or natural killer (NK) cells.
577. The method of any one of embodiments 450-576, wherein the engineered T cells are primary T cells or are progeny of primary immune cells, optionally wherein the progeny of primary immune cells are T cells or NK cells.
578. The method of any one of embodiments 450-577, wherein the engineered T cells comprise reduced expression of one or more MHC HLA class I molecules relative to an unaltered or unmodified wild-type or control cell.
579. The method of any one of embodiments 450-578, wherein the engineered T cells comprise reduced expression of one or more MHC HLA class II molecules relative to an unaltered or unmodified wild-type or control cell.
580. The method of any one of embodiments 450-579, wherein the engineered T cells comprise reduced expression of one or more MHC HLA class I molecules and of one or more MHC HLA class II molecules relative to an unaltered or unmodified wild-type or control cell.
581. The method of any one of embodiments 450-580, wherein the engineered T cells comprise reduced expression of B2M and/or CIITA relative to an unaltered or unmodified wild-type or control cell.
582. The method of embodiment 581, wherein the engineered T cells do not express B2M and/or CIITA.
583. The method of any one of embodiments 450-582, wherein the engineered T cells comprise reduced expression of TRAC and/or TRB.
584. The method of embodiment 583, wherein the engineered T cells do not express TRAC and/or TRB.
585. The method of any one of embodiments 450-584, wherein the engineered T cells comprise reduced expression of TRAC.
586. The method of embodiment 585, wherein the engineered T cells do not express TRAC.
587. The method of any one of embodiments 450-586, wherein the engineered T cells comprise reduced expression of TRB.
588. The method of embodiment 587, wherein the engineered T cells do not express TRB.
589. The method of any one of embodiments 450-588, wherein the engineered T cells comprise reduced expression of TRAC and TRB.
590. The method of any one of embodiments 450-589, wherein the one or more tolerogenic factors are selected from the group consisting of CD47, CD24. CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, and A20/TNFAIP3, CD39, CR1, HLA-F, IL15-RF, MANF, Serpinb9, optionally wherein the one or more tolerogenic factors comprise CD47.
591. The method of any one of embodiments 450-590, wherein the CD19-specific CAR and the one or more tolerogenic factors are encoded by a single bicistronic polynucleotide.
592. The method of anyone of embodiments 450-590, wherein the CD19-specific CAR and the CD47 are encoded by a single bicistronic polynucleotide.
593. The method of any one of embodiments 450-590, wherein the CD20-specific CAR and the one or more tolerogenic factors are encoded by a single bicistronic polynucleotide.
594. The method of any one of embodiments 450-590, wherein the CD20-specific CAR and the CD47 are encoded by a single bicistronic polynucleotide.
595. The method of any one of embodiments 450-594, wherein one or more of the first, second, and/or third exogenous polynucleotides or the bicistronic polynucleotide is inserted into a first, second, and/or third specific locus of at least one allele of the cell.
596. The method of embodiment 595, wherein the first, second and/or third specific loci are selected from the group consisting of a safe harbor locus, a target locus, an RHD locus, a 12M locus, a CIITA locus, a TRAC locus, and a TRB locus.
597. The method of embodiment 596, wherein the safe harbor locus is selected from the group consisting of a CCR5 locus, a PPPIR12C locus, a CLYBL locus, and a Rosa locus.
598. The method of embodiment 596, wherein the target locus is selected from the group consisting of a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, and a KDM5D locus.
599. The method of any one of embodiments 450-598, wherein the first, second, and/or third exogenous polynucleotide or the bicistronic polynucleotide is introduced into the engineered T cells using a gene therapy vector or a transposase system selected from the group consisting of transposases, PiggyBac transposons, Sleeping Beauty (SB11) transposons, Mos1 transposons, and To12 transposons.
600. The method of embodiments 599, wherein the gene therapy vector is a retrovirus or a fusosome.
601. The method of any one of embodiments 450-600, wherein the first, second. and/or third exogenous polynucleotide or the bicistronic polynucleotide is introduced into the engineered T cells using CRISPR/Cas gene editing.
602. The method of embodiment 601, wherein the CRISPR/Cas system comprises a Cas effector protein selected from the group consisting of Cas9, Cas12a, and Cas12b.
603. The method of embodiment 602, wherein the CRISPR/Cas system comprises a Cas effector protein selected from the group consisting of:
   (a) optionally selected from the group consisting of Cas3, Cas8a, Cas5, Cas8b, Cas8c, Cas10d, Cse1, Cse2, Csy1, Csy2, Csy3, and GSU0054;
   (b) optionally selected from the group consisting of Cas9, Csn2, and Cas4;
   (c) optionally selected from the group consisting of Cas10, Csm2, Cmr5, Cas10, Csx11, and Csx10;
   (d) optionally Csf1;
   (e) optionally selected from the group consisting of Cas12a, Cas12b, Cas12c, C2c4, C2c8, C2c5, C2c10, C2c9, CasX (Cas12e), and CasY (Cas12d); and
   (f) optionally selected from the group consisting of Cas13, Cas13a, C2c2, Cas13b, Cas13c, and Cas13d.
604. The method of any one of embodiments 601-603, wherein the CRISPR/Cas gene editing is carried out ex vivo from a donor subject.
605. The method of any one of embodiments 601-604, wherein the first, second, and/or third exogenous polynucleotide or the bicistronic polynucleotide is introduced into the engineered T cells using a lentiviral vector.
606. The method of any one of embodiments 450-605, wherein the engineered T cells evade NK cell mediated cytotoxicity upon administration to the patient.
607. The method of any one of embodiments 450-606, wherein the engineered T cells are protected from cell lysis by mature NK cells upon administration to the patient.
608. The method of any one of embodiments 450-607, wherein the engineered T cells evade macrophage-mediated cytotoxicity, optionally wherein the macrophage-mediated cytotoxicity involves phagocytosis and/or reactive oxygen species.
609. The method of any one of embodiments 450-608, wherein the engineered T cells do not induce an immune response to the cell upon administration to the patient.
610. The method of anyone of embodiments 450-609, wherein the administration is selected from the group consisting of intravenous injection, intramuscular injection, intravascular injection, and transplantation.
611. The method of anyone of embodiments 450-610, wherein the engineered T cells are administered before, during or after starting a different treatment regimen for the patient.
612. The method of embodiment 611, wherein the different treatment regimen is selected from the group consisting of re-dosing of the same or different cells, and pre-treatment, concurrent treatment, or subsequent treatment with an additional agent.
613. The method of embodiment 612, wherein the different cells are autologous T or NK cells or CAR-T cells expressing a first CAR that is different from a second CAR expressed by the engineered CAR-T cells.
614. The method of any one of embodiments 450-613, wherein the patient was treated with an immunodepleting therapy prior to administering the engineered T cells.
615. The method of embodiment 614, wherein the immunodepleting therapy comprises administration of fludarabine and/or cyclophosphamide.
616. The method of any one of embodiments 450-615, wherein the patient has undergone a prior antibody therapy.
617. The method of embodiment 616, wherein the antibody therapy is rituximab.
618. The method of embodiment 614, wherein the immunodepleting therapy comprises IV infusion of about 1-50 mg/m2 of fludarabine for about 1-7 days.
619. The method of embodiment 614, wherein the immunodepleting therapy comprises IV infusion of about 1, about 5, about 10, about 20, about 30, about 40, or about 50 mg/m2 of fludarabine for about 1, about 2, about 3, about 4, about 5, about 6, or about 7 days. is 620. The method of embodiment 614, wherein the immunodepleting therapy comprises IV infusion of about 30 mg/m2 of fludarabine for about 4 days.
621. The method of embodiment 614, wherein the immunodepleting therapy comprises IV infusion of about 100-1000 mg/m2 of cyclophosphamide for about 1-7 days.
622. The method of embodiment 621, wherein the immunodepleting therapy comprises IV infusion of about 100, about 200, about 300, about 400, about 500, about 600, about 700, about 800, about 900, about 1000 mg/m2 of cyclophosphamide for about 1, about 2, about 3, about 4, about 5, about 6, or about 7 days.
623. The method of embodiment 621 or 622, wherein the immunodepleting therapy comprises IV infusion of about 500 mg/m2 of cyclophosphamide for about 2 days.
624. The method of any one of embodiments 450-623, wherein at least about 40×104 engineered T cells are administered to the patient.
625. The method of any one of embodiments 450-624, wherein at least about 40×105 engineered T cells are administered to the patient.
626. The method of any one of embodiments 450-625, wherein the engineered T cells persist in the subject for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.
627. The method of any one of embodiments 450-626, wherein the therapeutic effect of the engineered T cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.
628. The method of any one of embodiments 450-627, wherein the wild type cell or the control cell is a starting material.
629. Use of a population of engineered T cells for treating cancer in a patient that is suspected of having cancer or has been diagnosed with cancer, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
630. Use of a population of engineered T cells for treating cancer in a patient that is suspected of having cancer or has been diagnosed with cancer, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
631. Use of a population of engineered T cells for treating cancer in a patient that is suspected of having cancer or has been diagnosed with cancer, wherein the engineered T cells comprise reduced expression of B2M relative to an unaltered or unmodified wild-type or control cell, a first exogenous polynucleotide encoding CD47, and a second exogenous polynucleotide encoding one or more CARs. wherein the one or more CARs comprise an extracellular ligand-binding domain having specificity for CD19, a hinge domain, a transmembrane domain, a co-stimulatory domain, and an intracellular signaling domain.
632. Use of a population of engineered T cells for treating cancer in a patient that is suspected of having cancer or has been diagnosed with cancer, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
633. Use of a population of engineered T cells for treating cancer in a patient that is suspected of having cancer or has been diagnosed with cancer, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
634. Use of a population of engineered T cells for treating cancer in a patient that is suspected of having cancer or has been diagnosed with cancer, wherein the engineered T cells comprise reduced expression of one or more MHC class I and/or class II HLA molecules relative to an unaltered or unmodified wild-type or control cell, reduced expression of TCR-alpha (TRAC) and/or TCR-beta (TRB) relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, a hinge domain, a transmembrane domain, a co-stimulatory domain, and an intracellular signaling domain.
635. Use of a population of engineered T cells for treating cancer in a patient that is suspected of having cancer or has been diagnosed with cancer, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous to polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
636. Use of a population of engineered T cells for treating cancer in a patient that is suspected of having cancer or has been diagnosed with cancer, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
637. Use of a population of engineered T cells for treating cancer in a patient that is suspected of having cancer or has been diagnosed with cancer, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular hgand-binding domain having specificity for CD19, a hinge domain, a transmembrane domain, a co-stimulatory domain, and an intracellular signaling domain.
638. Use of a population of engineered T cells for treating cancer in a patient that is suspected of having cancer or has been diagnosed with cancer, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
639. Use of a population of engineered T cells for treating cancer in a patient that is suspected of having cancer or has been diagnosed with cancer, wherein the engineered T cells comprise reduced expression of one or more MHC class I and/or class II HLA molecules relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
640. Use of a population of engineered T cells for treating cancer in a patient that is suspected of having cancer or has been diagnosed with cancer, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, a hinge domain, a transmembrane domain, a co-stimulatory domain, and an intracellular signaling domain.
641. Use of a population of engineered T cells for treating cancer in a patient that is suspected of having cancer or has been diagnosed with cancer, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, a hinge domain, a transmembrane domain, a co-stimulatory domain, and an intracellular signaling domain.
642. Use of a population of engineered T cells for treating multiple sclerosis in a patient that is suspected of having multiple sclerosis or has been diagnosed with multiple sclerosis, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
643. Use of a population of engineered T cells for treating multiple sclerosis in a patient that is suspected of having multiple sclerosis or has been diagnosed with multiple sclerosis, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
644. Use of a population of engineered T cells for treating multiple sclerosis in a patient that is suspected of having multiple sclerosis or has been diagnosed with multiple sclerosis, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
645. Use of a population of engineered T cells for treating multiple sclerosis in a patient that is suspected of having multiple sclerosis or has been diagnosed with multiple sclerosis, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
646. Use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
647. Use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, reduced expression of TCR-alpha (TRAC) and/or TCR-beta (TRB) relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, a hinge domain, a transmembrane domain, a co-stimulatory domain, and an intracellular signaling domain.
648. Use of a population of engineered T cells for treating multiple sclerosis in a patient that is suspected of having multiple sclerosis or has been diagnosed with multiple sclerosis, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
649. Use of a population of engineered T cells for treating multiple sclerosis in a patient that is suspected of having multiple sclerosis or has been diagnosed with multiple sclerosis, wherein the engineered T cells comprise reduced expression of B2M and CIITA relative to an unaltered or unmodified wild-type or control cel, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
650. Use of a population of engineered T cells for treating multiple sclerosis in a patient that is suspected of having multiple sclerosis or has been diagnosed with multiple sclerosis, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
651. Use of a population of engineered T cells for treating multiple sclerosis in a patient that is suspected of having multiple sclerosis or has been diagnosed with multiple sclerosis, wherein the engineered T cells comprise reduced expression of B2M and CIITA relative to an unaltered or unmodified wild-type or control cel, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
652. Use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain.
653. Use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, a hinge domain, a transmembrane domain, a co-stimulatory domain, and an intracellular signaling domain.
654. Use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, a hinge domain, a transmembrane domain, a co-stimulatory domain, and an intracellular signaling domain.
655. The use according to any one of embodiments 629-654, wherein the one or more CARs comprise a CD8a hinge domain, a CD28 hinge domain, or an IgG4 hinge domain.
656. The use according to embodiment 655, wherein the one or more CARs comprise a CD8a hinge domain having the amino acid sequence of SEQ ID NO: 34.
657. The use according to embodiment 655, wherein the one or more CARs comprise a CD28 hinge domain having the amino acid sequence of SEQ ID NO: 35 or 36.
658. The use according to embodiment 655, wherein the one or more CARs comprise a IgG4 hinge domain having the amino acid sequence of SEQ ID NO: 37, 38, or 39.
659. The use according to any one of embodiments 629-655, wherein the one or more CARs comprise a CD8a transmembrane domain or a CD28 transmembrane domain.
660. The use according to embodiment 659, wherein the one or more CARs comprise a CD8a transmembrane domain having the amino acid sequence of SEQ ID NO: 40.
661. The use according to embodiment 659, wherein the one or more CARs comprise a CD28 transmembrane domain having the amino acid sequence of SEQ ID NO: 41 or 42.
662. The use according to any one of embodiments 629-661, wherein the one or more CARs comprise a 4-168 costimulatory domain, a CD28 costimulatory domain, or a CD3C signaling domain.
663. The use according to embodiment 662, wherein the one or more CARs comprise a 4-1BB costimulatory domain having the amino acid sequence of SEQ ID NO: 43.
664. The use according to embodiment 662, wherein the one or more CARs comprise a CD28 costimulatory domain having the amino acid sequence of SEQ ID NO: 44 or 45.
665. The use according to embodiment 662, wherein the one or more CARs comprise a CD34 signaling domain having the amino acid sequence of SEQ ID NO: 46 or 47.
666. The use according to any one of embodiments 629-665, wherein the one or more CARs comprise an extracellular ligand-binding domain comprising an scFv sequence of any one of SEQ ID NOs: 25, 26, 27, 228, 229, or 230, or wherein the CARs have an scFv sequence comprising the heavy and light chain sequences of any one of SEQ ID NOs: 19-21 or 22-24.
667. The use according to any one of embodiments 629-666, wherein the one or more CARs have a sequence of any one of SEQ ID NOs: 232, 234, 236, 238, 240, or 242.
668. Use of a population of engineered T cells for treating multiple sclerosis in a patient that is suspected of having multiple sclerosis or has been diagnosed with multiple sclerosis, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD19 CAR having the HCDR sequences of Table 7 or Table 9 and LCDR sequences of Table 8 or Table 10.
669. Use of a population of engineered T cells for treating multiple sclerosis in a patient that is suspected of having multiple sclerosis or has been diagnosed with multiple sclerosis, wherein the engineered T cells comprise an exogenous polynucleotide encoding one or more CARs wherein the one or more CARs have a sequence of any one of SEO ID NOs: 232, 234, 236, 238, 240, or 242, or wherein the one or more CARs have an scFv sequence of any one of SEQ ID NOs: 25, 26, 27, 228, 229, or 230, or wherein the CARs have an scFv sequence comprising the heavy and light chain sequences of any one of SEQ ID NOs: 19-21 or 22-24.
670. The use of any one of embodiments 629-669, further comprising evaluating the patient for and/or diagnosing the patient with EBV infection and optionally multiple sclerosis prior to administering the population of engineered T cells to the patient.
671. The use of embodiment 870, wherein the diagnosis comprises evaluating the patient for EBV infection.
672. The use of embodiment 670 or 671, wherein the diagnosis comprises evaluating the patient for multiple sclerosis.
673. The use of any one of embodiments 629-672, wherein the treatment prevents multiple sclerosis.
674. The use of any one of embodiments 629-672, wherein the treatment treats multiple sclerosis.
675. The use of any one of embodiments 629-674, wherein the patient with the EBV infection has been diagnosed with multiple sclerosis.
676. The use of any one of embodiments 629-675, wherein the multiple sclerosis is relapsing-remitting multiple sclerosis, progressive relapsing multiple sclerosis, primary progressive multiple sclerosis, or secondary progressive multiple sclerosis.
677. The use of any one of embodiments 629-676, wherein the patient undergoes remission of multiple sclerosis following administration of the engineered T cells.
678. The use of any one of embodiments 629-677, wherein the patient with the EBV infection is undergoing treatment for the EBV infection.
679. The use of any one of embodiments 629-678, wherein the patient with the EBV infection has an active EBV infection.
680. The use of any one of embodiments 629-679, wherein the patient with the EBV infection has an inactive EBV infection.
681. The use of any one of embodiments 629-680, wherein the patient undergoes a reduced EBV infection following administration of the engineered T cells, optionally wherein the reduced EBV infection is characterized by reduced viral load.
682. The use of any one of embodiments 629-680, wherein the treatment prevents an EBV infection change from an inactive to an active EBV infection.
683. The use of any one of embodiments 629-682, wherein the use results in B cell depletion.
684. The use of any one of embodiments 629-683, wherein the engineered T cells comprise one or more of a CD19-specific CAR, a CD20-specific CAR, a CD22-specific CAR, a BCMA-specific CAR, a GPRCSD-specific CAR, a CD38-specific CAR, a CD70-specific CAR, a CD79b-specific CAR, and an EBV antigen-specific CAR.
685. Use of a population of engineered T cells for treating an autoimmune disease in a patient that is suspected of having the autoimmune disease or has been diagnosed with the autoimmune disease, wherein the engineered T cells comprise an exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
686. Use of a population of engineered T cells for treating an autoimmune disease in a patient that is suspected of having the autoimmune disease or has been diagnosed with the autoimmune disease, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
687. Use of a population of engineered T cells for treating an autoimmune disease in a patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
688. Use of a population of engineered T cells for treating an autoimmune disease in a patient that is suspected of having the autoimmune disease or has been diagnosed with the autoimmune disease, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
689. Use of a population of engineered T cells for treating an autoimmune disease in a patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
690. Use of a population of engineered T cells for treating an autoimmune disease in a patient that is suspected of having the autoimmune disease or has been diagnosed with the autoimmune disease, wherein the engineered T cells comprise reduced expression of 82M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, a hinge domain, a transmembrane domain, a co-stimulatory domain, and an intracellular signaling domain, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
691. The use according to any one of embodiments 629-690, wherein the one or more CARs comprise a CD8a hinge domain, a CD28 hinge domain, or an IgG4 hinge domain.
692. The use according to embodiment 691, wherein the one or more CARs comprise a CD8c hinge domain having the amino acid sequence of SEQ ID NO: 34.
693. The use according to embodiment 691, wherein the one or more CARs comprise a CD28 hinge domain having the amino acid sequence of SEQ ID NO: 35 or 36.
694. The use according to embodiment 691, wherein the one or more CARs comprise a IgG4 hinge domain having the amino acid sequence of SEQ ID NO: 37, 38, or 39.
695. The use according to any one of embodiments 629-694, wherein the one or more CARs comprise a CD8a transmembrane domain or a CD28 transmembrane domain.
696. The use according to embodiment 695, wherein the one or more CARs comprise a CD8a transmembrane domain having the amino acid sequence of SEQ ID NO: 40.
697. The use according to embodiment 695, wherein the one or more CARs comprise a CD28 transmembrane domain having the amino acid sequence of SEQ ID NO: 41 or 42.
698. The use according to any one of embodiments 629-697, wherein the one or more CARs comprise a 4-1BB costimulatory domain, a CD28 costimulatory domain, or a CD3(signaling domain.
699. The use according to embodiment 698, wherein the one or more CARs comprise a 4-1BB costimulatory domain having the amino acid sequence of SEQ ID NO: 43.
700. The use according to embodiment 698, wherein the one or more CARs comprise a CD28 costimulatory domain having the amino acid sequence of SEQ ID NO: 44 or 45.
701. The use according to embodiment 698, wherein the one or more CARs comprise a CD3C signaling domain having the amino acid sequence of SEQ ID NO: 46 or 47.
702. The use according to any one of embodiments 629-701, wherein the one or more CARs comprise an extracellular ligand-binding domain comprising an scFv sequence of any one of SEQ ID NOs: 25, 26, 27, 228.229, or 230, or wherein the CARs have an scFv sequence comprising the heavy and light chain sequences of any one of SEQ ID NOs: 19-21 or 22-24.
703. The use according to any one of embodiments 629-702, wherein the one or more CARs have a sequence of any one of SEQ ID NOs: 232, 234, 236, 238, 240, or 242.
704. Use of a population of engineered T cells for treating an autoimmune disease in a patient that is suspected of having the autoimmune disease or has been diagnosed with the autoimmune disease, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the HCDR sequences of Table 7 or Table 9 and LCDR sequences of Table 8 or Table 10, and wherein the autoimmune disease is selected from group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
705. The use according to embodiment 704, further comprising evaluating the patient for and/or diagnosing the patient with the autoimmune disease.
706. The use of any one of embodiments 704 or 705, wherein the encoded CD19 CAR has a corresponding amino acid sequence set forth in SEQ ID NOs: 232, 234, 236, 238, 240, or 242 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 232, 234, 236, 238, 240, or 242, with the following components: CD8α signal peptide, FMC63 scFv (VL-Whitlow linker-VH), CD8a hinge domain, CD8a transmembrane domain, 4-188 costimulatory domain, and CD3C signaling domain.
707. Use of a population of engineered T cells for treating an autoimmune disease in a patient that is suspected of having the autoimmune disease or has been diagnosed with the autoimmune disease, wherein the engineered T cells comprise an exogenous polynucleotide encoding one or more CARs wherein the one or more CARs have a sequence of any one of SEQ ID NOs: 232, 234, 236, 238, 240, or 242, or wherein the one or more CARs have an scFv sequence of any one of SEQ ID NOs: 25, 28, 27, 228, 229, or 230.
708. Use of a population of engineered T cells for treating an autoimmune disease in a patient that is suspected of having the autoimmune disease or has been diagnosed with the autoimmune disease, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 232, 234, 236, 238, 240, or 242, or wherein the one or more CARs have an scFv sequence of any one of SEQ ID NOs: 25. 26, 27, 228, 229, or 230.
709. Use of a population of engineered T cells for treating an autoimmune disease in a patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 232, 234, 236, 238, 240, or 242, or wherein the one or more CARs have an scFv sequence of any one of SEQ ID NOs: 25, 26, 27, 228, 229, or 230.
710. Use of a population of engineered T cells for treating an autoimmune disease in a patient that is suspected of having the autoimmune disease or has been diagnosed with the autoimmune disease, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 232, 234, 236, 238, 240, or 242, or wherein the one or more CARs have an scFv sequence of any one of SEQ ID NOs: 25, 26, 27, 228. 229, or 230.
711. Use of a population of engineered T cells for treating an autoimmune disease in a patient that is suspected of having the autoimmune disease or has been diagnosed with the autoimmune disease, wherein the engineered T cells comprise reduced expression of 82M relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 232, 234, 236, 238, 240, or 242, or wherein the one or more CARs have an scFv sequence of any one of SEQ ID NOs: 25, 26, 27, 228, 229, or 230.
712. Use of a population of engineered T cells for treating an autoimmune disease in a patient that is suspected of having the autoimmune disease or has been diagnosed with the autoimmune disease, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 232, 234, 236, 238, 240, or 242, or wherein the one or more CARs have an scFv sequence of any one of SEQ ID NOs: 25, 26, 27, 228, 229, or 230.
713. Use of a population of engineered T cells for treating an autoimmune disease in a patient that is suspected of having the autoimmune disease or has been diagnosed with the autoimmune disease, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs wherein the one or more CARs have a sequence of any one of SEQ ID NOs: 232. 234. 236, 238, 240, or 242, or wherein the one or more CARs have an scFv sequence of any one of SEQ ID NOs: 25, 26, 27, 228, 229, or 230.
714. The use of any one of embodiments 629-713, further comprising evaluating the patient for and/or diagnosing the patient with the autoimmune disease prior to administering the population of engineered T cells to the patient.
715. The use of any one of embodiments 629-714, wherein the autoimmune disease is selected from the group consisting of lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
716. The use of any one of embodiments 629-715, wherein the patient is suspected of having an EBV infection or has been diagnosed as having an EBV infection.
717. The use of any one of embodiments 629-716, wherein the patient is suspected of having multiple sclerosis or has been diagnosed with multiple sclerosis.
718. The use of any one of embodiments 629-717, further comprising administering a second, third, fourth, fifth, or sixth dose of the engineered T cells to the patient.
719. The use of embodiment 718, wherein the CAR is the same in one or more of the first, second, third, fourth, fifth, and/or sixth dose of engineered T cells.
720. The use of embodiment 718, wherein the CAR is different in one or more of the first, second, third, fourth, fifth, and/or sixth dose of engineered T cells.
721. The use of any one of embodiments 629-720, wherein the CAR has an scFv sequence of any one of SEQ ID NOs: 25, 26, 27, 228, 229, or 230, or wherein the CARs have an scFv sequence comprising the heavy and light chain sequences of any one of SEQ ID NOs: 19-21 or 22-24.
722. The use of any one of embodiments 629-721, wherein the CAR has a sequence of any one of SEQ ID Nos: 232, 234, 236, 238, 240, or 242.
723. The use of any one of embodiments 629-722, wherein the engineered T cells comprise a CD19-specific CAR and a CD20-specific CAR.
724. The use of embodiment 723, wherein the CD19-specific CAR and the CD20-specific CAR are encoded by a single bicistronic polynucleotide.
725. The use of embodiment 723, wherein the CD19-specific CAR and the CD20-specific CAR are encoded by a single bispecific CAR.
726. The use of embodiment 723, wherein the CD19-specific CAR and the CD20-specific CAR are encoded by two separate polynucleotides.
727. The use of any one of embodiments 723-726, wherein the CD19 CAR T cells and CD20 CAR T cells are administered concomitantly.
728. The use of anyone of embodiments 723-726, wherein the CD19 CAR+ T cells and CD20 CAR+ T cells are administered sequentially.
729. The use of embodiment 728, wherein the CD19 CAR+ T cells are administered prior to administration of the CD20 CAR+ T cells.
730. The use of embodiment 728, wherein the CD20 CAR+ T cells are administered prior to administration of the CD19 CAR+ T cells.
731. The use of any one of embodiments 723-730, wherein the number of cells administered as a therapeutically effective amount of the CD19 and/or CD20 CAR T cells is greater than the number of cells administered as a therapeutically effective amount of CD19 CAR T cells or CD20 CAR T cells alone.
732. The use of any one of embodiments 723-730, wherein the number of cells administered to as a therapeutically effective amount of the CD19 and/or CD20 CAR T cells is less than the number of cells administered as a therapeutically effective amount of CD19 CAR T cells or CD20 CAR T cells alone.
733. The use of any one of embodiments 829-733, wherein the engineered T cells comprise an EBV antigen-specific CAR and a CD20-specific CAR.
734. The use of embodiment 733, wherein the EBV antigen-specific CAR and the CD20-specific CAR are encoded by a single bicistronic polynucleotide.
735. The use of embodiment 733, wherein the EBV antigen-specific CAR and the CD20-specific CAR are encoded by a single bispecific CAR.
736. The use of embodiment 733, wherein the EBV antigen-specific CAR and the CD20-specific CAR are encoded by two separate polynucleotides.
737. The use of any one of embodiments 733-736, wherein the EBV antigen CAR T cells and CD20 CAR T cells are administered concomitantly.
738. The use of any one of embodiments 733-736, wherein the EBV antigen CAR+ T cells and CD20 CAR+ T cells are administered sequentially.
739. The use of embodiment 738, wherein the EBV antigen CAR+ T cells are administered prior to administration of the CD20 CAR+ T cells.
740. The use of embodiment 738, wherein the CD20 CAR+ T cells are administered prior to administration of the EBV antigen CAR+ T cells.
741. The use of any one of embodiments 733-740, wherein the number of cells administered as a therapeutically effective amount of the EBV antigen and/or CD20 CAR T cells is greater than the number of cells administered as a therapeutically effective amount of EBV antigen CAR T cells or CD20 CAR T cells alone.
742. The use of any one of embodiments 733-741, wherein the number of cells administered to as a therapeutically effective amount of the EBV antigen and/or CD20 CAR T cells is less than the number of cells administered as a therapeutically effective amount of EBV antigen CAR T cells or CD20 CAR T cells alone.
743. The use of any one of embodiments 629-742, wherein the engineered T cells comprise an EBV antigen-specific CAR and a CD19-specific CAR.
744. The use of embodiment 743, wherein the EBV antigen-specific CAR and the CD19-specific CAR are encoded by a single bicistronic polynucleotide.
745. The use of embodiment 743, wherein the EBV antigen-specific CAR and the CD19-specific CAR are encoded by a single bispecific CAR.
746. The use of embodiment 743, wherein the EBV antigen-specific CAR and the CD19-specific CAR are encoded by two separate polynucleotides.
747. The use of any one of embodiments 743-746, wherein the EBV antigen CAR T cells and CD19 CAR T cells are administered concomitantly.
748. The use of any one of embodiments 743-746, wherein the EBV antigen CAR+ T cells and CD19 CAR+ T cells are administered sequentially.
749. The use of embodiment 748, wherein the EBV antigen CAR+ T cells are administered prior to administration of the CD19 CAR+ T cells.
750. The use of embodiment 748, wherein the CD19 CAR+ T cells are administered prior to administration of the EBV antigen CAR+ T cells.
751. The use of any one of embodiments 743-750. wherein the number of cells administered as a therapeutically effective amount of the EBV antigen and/or CD19 CAR T cells is greater than the number of cells administered as a therapeutically effective amount of EBV antigen CAR T cells or CD19 CAR T cells alone.
752. The use of any one of embodiments 743-750, wherein the number of cells administered to as a therapeutically effective amount of the EBV antigen and/or CD19 CAR T cells is less than the number of cells administered as a therapeutically effective amount of EBV antigen CAR T cells or CD19 CAR T cells alone.
753. The use of any one of embodiments 629-752, wherein the engineered T cells comprise an EBV antigen-specific CAR and a CD22-specific CAR.
754. The use of embodiment 754, wherein the EBV antigen-specific CAR and the CD22-specific CAR are encoded by a single bicistronic polynucleotide.
755. The use of embodiment 754, wherein the EBV antigen-specific CAR and the CD22-specific CAR are encoded by a single bispecific CAR.
756. The use of embodiment 754, wherein the EBV antigen-specific CAR and the CD22-specific CAR are encoded by two separate polynucleotides.
757. The use of any one of embodiments 754-756, wherein the EBV antigen CAR T cells and CD22 CAR T cells are administered concomitantly.
758. The use of any one of embodiments 754-756, wherein the EBV antigen CAR+ T cells and CD22 CAR+ T cells are administered sequentially.
759. The use of embodiment 758, wherein the EBV antigen CAR+ T cells are administered prior to administration of the CD22 CAR+ T cells.
760. The use of embodiment 759, wherein the CD22 CAR+ T cells are administered prior to administration of the EBV antigen CAR+ T cells.
761. The use of any one of embodiments 754-760, wherein the number of cells administered as a therapeutically effective amount of the EBV antigen and/or CD22 CAR T cells is greater than the number of cells administered as a therapeutically effective amount of EBV antigen CAR T cells or CD22 CAR T cells alone.
762. The use of any one of embodiments 754-760, wherein the number of cells administered to as a therapeutically effective amount of the EBV antigen and/or CD22 CAR T cells is less than the number of cells administered as a therapeutically effective amount of EBV antigen CAR T cells or CD22 CAR T cells alone.
763. The use of any one of embodiments 629-762, wherein the engineered T cells are primary T cells, are propagated from a primary T cell or a progeny thereof, or are derived from a T cell differentiated from an iPSC or a progeny thereof.
764. The use of any one of embodiments 629-762, wherein the engineered T cells are differentiated cells derived from an induced pluripotent stem cell or a progeny thereof.
765. The use of embodiment 764, wherein the differentiated cells are a T cells or natural killer (NK) cells.
766. The use of any one of embodiments 629-765, wherein the engineered T cells are primary T cells, are progeny of primary immune cells, optionally wherein the progeny of primary immune cells are T cells or NK cells.
767. The use of any one of embodiments 629-766, wherein the engineered T cells comprise reduced expression of one or more MHC HLA class I molecules relative to an unaltered or unmodified wild-type or control cell.
768. The use of any one of embodiments 629-767. wherein the engineered T cells comprise reduced expression of one or more MHC HLA class II molecules relative to an unaltered or unmodified wild-type or control cell.
769. The use of any one of embodiments 629-768, wherein the engineered T cells comprise reduced expression of one or more MHC HLA class I molecules and of one or more MHC HLA class II molecules relative to an unaltered or unmodified wild-type or control cell.
770. The use of any one of embodiments 629-769, wherein the engineered T cells comprise reduced expression of B2M and/or CIITA relative to an unaltered or unmodified wild-type or control cell.
771. The use of embodiment 770, wherein the engineered T cells do not express B2M and/or CIITA.
772. The use of any one of embodiments 629-771, wherein the engineered T cells comprise reduced expression of TRAC and/or TRB.
773. The use of embodiment 772, wherein the engineered T cells do not express TRAC and/or TRB.
774. The use of any one of embodiments 629-773, wherein the engineered T cells comprise reduced expression of TRAC.
775. The use of embodiment 774, wherein the engineered T cells do not express TRAC.
776. The use of any one of embodiments 629-775, wherein the engineered T cells comprise reduced expression of TRB.
777. The use of embodiment 776, wherein the engineered T cells do not express TRB.
778. The use of any one of embodiments 629-777, wherein the engineered T cells comprise reduced expression of TRAC and TRB.
779. The use of any one of embodiments 629-778, wherein the one or more tolerogenic factors are selected from the group consisting of CD47, CD24, CD27, C035, CD46, CD55, CD59, CD200, HLA-C, HLA-E, HLA-E heavy chain, HLA-G, PD-1, IDO1, CTLA4-Ig, C1-Inhibitor, IL-10, IL-35, FasL, CCL21, CCL22, Mfge8, A20/TNFAIP3, CD39, CR1, HLA-F, IL15-RF, MANF, and Serpinb9, optionally wherein the one or more tolerogenic factors comprise CD47.
780. The use of any one of embodiments 629-779, wherein the CD19-specific CAR and the one or more tolerogenic factors are encoded by a single bicistronic polynucleotide.
781. The use of any one of embodiments 629-779, wherein the CD19-specific CAR and the CD47 are encoded by a single bicistronic polynucleotide.
782. The use of any one of embodiments 629-779, wherein the CD20-specific CAR and the one or more tolerogenic factors are encoded by a single bicistronic polynucleotide.
783. The use of any one of embodiments 629-779, wherein the CD20-specific CAR and the CD47 are encoded by a single bicistronic polynucleotide.
784. The use of any one of embodiments 629-783, wherein one or more of the first, second, and/or third exogenous polynucleotides or the bicistronic polynucleotide is inserted into a first, second, and/or third specific locus of at least one allele of the cell.
785. The use of embodiment 784, wherein the first, second and/or third specific loci are selected from the group consisting of a safe harbor locus, a target locus, an RHD locus, a B2M locus, a CIITA locus, a TRAC locus, and a TRB locus.
786. The use of embodiment 785, wherein the safe harbor locus is selected from the group consisting of a CCR5 locus, a PPP1RI2C locus, a CLYBL locus, and a Rosa locus.
787. The use of embodiment 785, wherein the target locus is selected from the group consisting of a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, and a KDM5D locus.
788. The use of any one of embodiments 629-787, wherein the first, second, and/or third exogenous polynucleotide or the bicistronic polynucleotide is introduced into the engineered T cells using a gene therapy vector or a transposase system selected from the group consisting of transposases, PiggyBac transposons, Sleeping Beauty (SB11) transposons, Mos1 transposons, and Tol2 transposons.
789. The use of embodiments 788, wherein the gene therapy vector is a retrovirus or a fusosome.
790. The use of any one of embodiments 629-789, wherein the first, second, and/or third exogenous polynucleotide or the bicistronic polynucleotide is introduced into the engineered T cells using CRISPR/Cas gene editing.
791. The use of embodiment 790, wherein the CRISPR/Cas system comprises a Cas effector protein selected from the group consisting of Cas9. Cas12a, and Cas12b.
792. The use or dosage regimen of embodiment 791, wherein the CRISPR/Cas system compises a Cas effector protein selected from the group consisting of:
   (a) optionally selected from the group consisting of Cas3, Cas8a, Cas5, Cas8b, Cas8c, Cas10d, Cse1, Cse2, Csy1, Csy2, Csy3, and GSU0054;
   (b) optionally selected from the group consisting of Cas9, Csn2, and Cas4;
   (c) optionally selected from the group consisting of Cas10, Csm2, Cmr5, Cas10, Csx11, and Csx10;
   (d) optionally Csf1;
   (e) optionally selected from the group consisting of Cas12a, Cas12b. Cas12c, C2c4, C2c8, C2c5, C2c10, C2c9, CasX (Cas12e), and CasY (Cas12d); and
   (f) optionally selected from the group consisting of Cas13, Cas13a, C2c2, Cas13b, Cas13c, and Cas13d.
793. The use of any one of embodiments 790-792, wherein the CRISPR/Cas gene editing is caried out ex vivo from a donor subject.
794. The use of any one of embodiments 790-793, wherein the first, second, and/or third exogenous polynucleotide or the bicistronic polynucleotide is introduced into the engineered T cells using a lentiviral vector.
795. The use of any one of embodiments 629-794. wherein the engineered T cells evade NK cell mediated cytotoxicity upon administration to the patient.
796. The use of any one of embodiments 629-795, wherein the engineered T cells are protected from cell lysis by mature NK cells upon administration to the patient.
797. The use of any one of embodiments 629-796, wherein the engineered T cells evade macrophage-mediated cytotoxicity, optionally wherein the macrophage-mediated cytotoxicity involves phagocytosis and/or reactive oxygen species.
798. The use of any one of embodiments 629-797, wherein the engineered T cells do not induce an immune response to the cell upon administration to the patient.
799. The use of any one of embodiments 629-798, wherein the administration is selected from the group consisting of intravenous injection, intramuscular injection, intravascular injection, and transplantation.
800. The use of any one of embodiments 629-799, wherein the engineered T cells are administered before, during or after starting a different treatment regimen for the patient.
801. The use of embodiment 800, wherein the different treatment regimen is selected from the group consisting of re-dosing of the same or different cells, and pre-treatment, concurrent treatment, or subsequent treatment with an additional agent.
802. The use of embodiment 801, wherein the different cells are autologous T or NK cells or CAR-T cells expressing a first CAR that is different from a second CAR expressed by the engineered CAR-T cells.
803. The use of any one of embodiments 629-802, wherein the patient was treated with an immunodepleting therapy prior to administering the engineered T cells.
804. The use of embodiment 803, wherein the immunodepleting therapy comprises administration of fludarabine and/or cyclophosphamide.
805. The use of any one of embodiments 629-804. wherein the patient has undergone a prior antibody therapy.
806. The use of embodiment 805, wherein the antibody therapy is rituximab.
807. The use of embodiment 806, wherein the immunodepleting therapy comprises IV infusion of about 1-50 mg/m2 of fludarabine for about 1-7 days.
808. The use of embodiment 807, wherein the immunodepleting therapy comprises IV infusion of about 1, about 5, about 10, about 20, about 30, about 40, or about 50 mg/m2 of fludarabine for about 1, about 2, about 3, about 4, about 5, about 6, or about 7 days.
809. The use of embodiment 807 or 808, wherein the immunodepleting therapy comprises IV infusion of about 30 mg/m2 of fludarabine for about 4 days.
810. The use of embodiment 804, wherein the immunodepleting therapy comprises IV infusion of about 100-1000 mg/m2 of cyclophosphamide for about 1-7 days.
811. The use of embodiment 804, wherein the immunodepleting therapy comprises IV infusion of about 100, about 200, about 300, about 400, about 500, about 600, about 700, about 800, about 900, about 1000 mg/m2 of cyclophosphamide for about 1, about 2, about 3, about 4, about 5, about 6, or about 7 days.
812. The use of embodiment 810 or 811. wherein the immunodepleting therapy comprises IV infusion of about 500 mg/m2 of cyclophosphamide for about 2 days.
813. The use of any one of embodiments 629-812, wherein at least about 40×104 engineered T cells are administered to the patient.
814. The use of any one of embodiments 629-813, wherein at least about 40×105 engineered T cells are administered to the patient.
815. The use of any one of embodiments 629-814, wherein the engineered T cells persist in the subject for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.
816. The use of any one of embodiments 629-815, wherein the therapeutic effect of the engineered T cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.
817. The use of any one of embodiments 629-816, wherein the wild type cell or the control cell is a starting material.

The method of any one of embodiments 450-628 or the use of any one of embodiments 629-817, wherein the population of engineered T cells comprises an engineered T cell comprising one or more modifications that (i) reduce expression of one or more MHC class I molecules and/or one or more MHC class II molecules, and/or (ii) increase expression of one or more tolerogenic factors, wherein the reduced expression of (i) and the increased expression of (ii) is relative to a comparable T cell that does not comprise the modifications.
819. An engineered T cell comprising one or more modifications that (i) reduce expression of one or more MHC class I molecules and/or one or more MHC class II molecules, and/or (ii) increase expression of one or more tolerogenic factors, wherein the reduced expression of (i) and the increased expression of (ii) is relative to a comparable T cell that does not comprise the modifications.
820. A method comprising administering a population of engineered cells to the patient, wherein the population of engineered cells comprises an engineered cell comprising one or more modifications that (i) disrupt one or more MHC class I molecules and/or one or more MHC class II molecules, and/or (ii) increase expression of one or more tolerogenic factors, wherein the increased expression of (ii) is relative to a comparable cell that does not comprise the modifications.
821. The method of embodiment 820, wherein the method is a method of treating a patient who is suspected of having an autoimmune disease or who has been diagnosed with an autoimmune disease.
822. The method of embodiment 821, wherein the autoimmune disease is selected from the group consisting of Epstein Barr Virus (EBV), multiple sclerosis, lupus, systemic lupus erythematosus, lupus nephritis, extrarenal lupus, multiple sclerosis, systemic sclerosis, vasculitis, ANCA-vasculitis, Crohn's disease, Myasthenia Gravis, Stiff-Person syndrome, or a pulmonary condition.
823. The method of embodiment any one of embodiments 820-822, wherein the engineered cell is a T cell or an NK cell, optionally wherein the T cell is a cytotoxic T cells, helper T cells, memory T cells, central memory T cells, effector memory T cells, effector memory RA T cells, regulatory T cells, or a tissue infiltrating lymphocytes.
824. The method of any one of embodiments 820-823, wherein the engineered cell is a primary cell or a differentiated cell.
825. The method of any one of embodiments 820-824, wherein the one or more modifications that disrupt the one or more MHC class I molecules and/or one or more MHC class II molecules reduce expression of the one or more MHC class I molecules and/or one or more MHC class II molecules.
826. The method of any one of embodiments 820-825, wherein the one or more tolerogenic factors are selected from the group consisting of A20/TNFAIP3, C1-Inhibitor, CCL21, CCL22, CD16, CD16 Fc receptor, CD24, CD27, CD35, CD39, CD46, CD47, CD52, CD55, CD59. CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, MANF, Mfge8, PD-1, Serpinb9, and any combination thereof.
827. The method of any one of embodiments 820-828, wherein the engineered cells further comprise an exogenous polynucleotide encoding one or more chimeric antigen receptors (CARs), chimeric autoantibody receptors (CAARs), or chimeric B-cell autoantibody receptors (BARs).
828. The method of embodiment 827, wherein the one or more CARs comprise an extracellular ligand-binding domain having specificity for CD19, CD22, CD20, BCMA, an EBV antigen, CD27, CD30, CD19 and CD20, CD19 and CD22, CD19 and CD27, EBNA1, EBNA3A, BRLF1, BALF4, EBNA3C, LMP1, LMP2, LMP2A, LMP2B, BZLF1, BMLF1, gp350, gH/gL, EBNA1 and LMP1, EBNA1 and LMP2A, EBNA1 and LMP1 and LMP2A, LMP and BARF1 and EBNA1, CD19 and an EBV antigen, CD20 and an EBV antigen, or CD22 and an EBV antigen.
829. The method of embodiment 827 or 828, wherein the one or more CAARs comprise an antigen selected from the group consisting of a pancreatic β-cell antigen, synovial joint antigen, myelin basic protein, proteolipid protein, myelin oligodendritic glycoprotein, MuSK, keratinocyte adhesion protien desmoglein 3 (Dsg3), Ro-RNP complex, La antigen, myeloperoxidase, proteinase 3, cardiolipin, citrullinated proteins, carbamylated proteins, and a3 chain of basement membrane collagen.
830. The method of any one of embodiments 827-829, wherein the one or more BARs comprise an FVIII antigen.
831. The method of embodiment 827 or 828, wherein the autoimmune disease is EBV and wherein the one or more CARs comprise an extracellular ligand-binding domain having specificity for CD19, a hinge domain, a transmembrane domain, a co-stimulatory domain, and an intracellular signaling domain.
832. The method of embodiment 827 or 828, wherein the autoimmune disease is EBV and wherein the one or more CARs comprise an extracellular ligand-binding domain having specificity for CD19 or CD22, a hinge domain, a transmembrane domain, a co-stimulatory domain, and an intracellular signaling domain.
833. The method of embodiment 827 or 828, wherein the autoimmune disease is multiple sclerosis and wherein the one or more CARs comprise an extracellular ligand-binding domain having specificity for CD19, a hinge domain, a transmembrane domain, a co-stimulatory domain, and an intracellular signaling domain.
834. The method of embodiment 827 or 828, wherein the autoimmune disease is lupus nephritis and wherein the one or more CARs comprise an extracellular ligand-binding domain having specificity for CD19, a hinge domain, a transmembrane domain, a co-stimulatory domain, and an intracellular signaling domain.
835. The method of embodiment 827 or 828, wherein the autoimmune disease is extrarenal lupus and wherein the one or more CARs comprise an extracellular ligand-binding domain having specificity for CD19, a hinge domain, a transmembrane domain, a co-stimulatory domain, and an intracellular signaling domain.
836. The method of embodiment 827 or 828, wherein the autoimmune disease is ANCA-vasculitis and wherein the one or more CARs comprise an extracellular ligand-binding domain having specificity for CD19, a hinge domain, a transmembrane domain, a co-stimulatory domain, and an intracellular signaling domain.
837. The method of any one of embodiments 820-836, further comprising evaluating the patient for and/or diagnosing the patient with EBV, multiple sclerosis, lupus, systemic lupus erythematosus, multiple sclerosis, systemic sclerosis, vasculitis, Crohn's disease, Myasthenia Gravis, Stiff-Person syndrome, or a pulmonary condition.
838. The method of any one of embodiments 820-837, wherein the engineered cells further comprise one or more modifications that disrupt TCR-alpha (TRAC) and/or TCR-beta (TRB).
839. The method of embodiment 838, wherein the one or more modifications that disrupt TCR-alpha (TRAC) and/or TCR-beta (TRB) reduce expression of TCR-alpha (TRAC) and/or TCR-beta (TRB).
840. The method of any one of embodiments 820-839, further comprising administering a second therapeutic agent to the patient.
841. The method of embodiment 840, wherein the second therapeutic agent is an anti-BLyS therapy.
842. An engineered cell comprising one or more modifications that (i) disrupt one or more MHC class I molecules and/or one or more MHC class II molecules, and/or (ii) increase expression of one or more tolerogenic factors, wherein the increased expression of (ii) is relative to a comparable cell that does not comprise the modifications.
843. The engineered cell of embodiment 842, wherein the engineered cell is a T cell or an NK cell, optionally wherein the T cell is a cytotoxic T cells, helper T cells, memory T cels, central memory T cells, effector memory T cells, effector memory RA T cells, regulatory T cells, or a tissue infiltrating lymphocytes.
844. The engineered cell of embodiment 842 or embodiment 843, wherein the engineered cell is a primary cell or a differentiated cell.
845. The engineered cell of any one of embodiments 842-844, wherein the one or more modifications that disrupt the one or more MHC class I molecules and/or one or more MHC class II molecules reduce expression of the one or more MHC class I molecules and/or one or more MHC class II molecules.
848. The engineered cell of any one of embodiments 842-845, wherein the one or more tolerogenic factors are selected from the group consisting of A20/TNFAIP3, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, MANF, Mfge8, PD-L1, Serpinb9, and any combination thereof.
847. The engineered cell of any one of embodiments 842-846, wherein the engineered cell further comprises an exogenous polynucleotide encoding one or more chimeric antigen receptors (CARs), chimeric autoantibody receptors (CAARs), or chimeric B-cell autoantibody receptors (BARs).
848. The engineered cell of embodiment 847, wherein the one or more CARs comprise an extracellular ligand-binding domain having specificity for CD19, CD22, CD20, BCMA, an EBV antigen, CD27, CD30, CD19 and CD20, CD19 and CD22, CD19 and CD27, EBNA1, EBNA3A, BRLF1, BALF4, EBNA3C, LMP1, LMP2, LMP2A, LMP2B, BZLF1, BMLF1, gp350, gH/gL, EBNA1 and LMP1, EBNA1 and LMP2A, EBNA1 and LMP1 and LMP2A, LMP and BARF1 and EBNA1, CD19 and an EBV antigen, CD20 and an EBV antigen, or CD22 and an EBV antigen.
849. The engineered cell of embodiment 847 or 848, wherein the one or more CAARs comprise an antigen selected from the group consisting of a pancreatic β-cell antigen, synovial joint antigen, myelin basic protein, proteolipid protein, myelin oligodendritic glycoprotein, MuSK, keratinocyte adhesion protein desmoglein 3 (Dsg3), Ro-RNP complex, La antigen, myeloperoxidase, proteinase 3, cardiolipin, citrullinated proteins, carbamylated proteins, and a3 chain of basement membrane collagen.
850. The engineered cell of any one of embodiments 847-849, wherein the one or more BARs comprise an FVIII antigen.
851. The engineered cell of any one of embodiments 842-850, wherein the engineered cell further comprises one or more modifications that disrupt TCR-alpha (TRAC) and/or TCR-beta (TRB).
852. The engineered cell of embodiment 851, wherein the one or more modifications that disrupt TCR-alpha (TRAC) and/or TCR-beta (TRB) reduce expression of TCR-alpha (TRAC) and/or TCR-beta (TRB).

### Tables

TABLE 1
Boundaries of CDRs according to various numbering schemes

| CDR | Kabat | Chothia | AbM | Contact |
|---|---|---|---|---|
| CDR-H1 | H31—H35B | H26—H32 . . . 34 | H26—H35B | H30—H35B |
| (Kabat | | | | |
| Numbering¹) | | | | |
| CDR-H1 | H31—H35 | H26—H32 | H26—H35 | H30—H35 |
| (Chothia | | | | |
| Numbering²) | | | | |
| CDR-H2 | H50—H65 | H52—H56 | H50—H58 | H47—H58 |
| CDR-H3 | H95—H102 | H95—H102 | H95—H102 | H93—H101 |

¹Kabat et al. (1991), “Sequences of Proteins of Immunological Interest,” 5^{th} Ed. Public Health Service, National Institutes of Health, Bethesda, MD
²Al-Lazikani et al., (1997) JMB 273,927-948

TABLE 2
Amino Acid Exemplary Substitutions

| Original | |
|---|---|
| Residue | Exemplary Substitution |
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; |
| | Norleucine |
| Leu (L) | Norleucine; Ile; Val; Met; Ala; |
| | Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; |
| | Norleucine |

TABLE 3
HCDRS in Kabat Numbering Scheme

| CD19 | H-CDR1 | | H-CDR2 | | H-CDR3 | |
|---|---|---|---|---|---|---|
| Binder | Sequence | SEQ ID NO: | Sequence | SEQ ID NO: | Sequence | SEQ ID NO: |
| 1 | SNYMS | 1 | VIYSGGSTYYADSVKG | 4 | DGGLDTLYGMDV | 7 |
| 2 | DYYMS | 2 | YISSSGSTTYYADSVKG | 5 | VEGWSLLDY | 8 |
| 3 | SYWIG | 3 | IIYPGDSDTRYSPSFQG | 6 | LPFVGATTGGFDY | 9 |

TABLE 4
LCDRS in Kabat Numbering Scheme

| CD19 | L-CDR1 | | L-CDR2 | | L-CDR3 | |
|---|---|---|---|---|---|---|
| Binder | Sequence | SEQ ID NO: | Sequence | SEQ ID NO: | Sequence | SEQ ID NO: |
| 1 | TGSSSNIGAGYDVH | 10 | GNSNRPS | 13 | QSYDSSLSGSYV | 16 |
| 2 | SGSSSNIGNNYVS | 11 | DNNKRPS | 14 | GTWDSSLGAVV | 17 |
| 3 | TGTSSDVGGYNYVS | 12 | EVSNRPS | 15 | SSYTSSSTLV | 18 |

TABLE 5
Sequences

| CD19 | | |
|---|---|---|
| Binder | Sequence | SEQ ID NO: |
| 1 | EVQLVESGGGLIQPGGSLRLSCAASGFTVSSNYMSWVRQAPGKGLEWVSVIY | 19 |
| | SGGSTYYADSVKGRFTISRDNSKNTLYLQMNSLRAEDTAVYYCARDGGLDTLY | |
| | GMDVWGQGTTVTVSS | |
| 2 | EVHLVESGGGLVQPGGSLRLSCAASGFTFSDYYMSWIRQAPGKGLEWVSYISS | 20 |
| | SGSTTYYADSVKGRFTISRDNAKNSLYLQMNSLRAEDTAVYYCARVEGWSLLD | |
| | YWGQGTLVTVSS | |
| 3 | EVQLVQSGAEVKKPGESLKISCKGSGYSFTSYWIGWVRQMPGKGLEWM | 21 |
| | GHIYPGDSDTRYSPSFQGQVTISADKSISTAYLQWSSLKASDTAMYYCARL | |
| | PFVGATTGGFDYWGQGTLVTVSS | |

TABLE 6
VL Sequences

| CD19 | | |
|---|---|---|
| Binder | Sequence | SEQ ID NO: |
| 1 | QSVVTQPPSVSGAPGQRVTISCTGSSSNIGAGYDVHWYQQLPGTAPKLLI | 22 |
| | YGNSNRPSGVPDRFSGSKSGTSASLAITGLQAEDEADYYCQSYDSSLSGSY | |
| | VFGTGTKVTVL | |
| 2 | QSVLTQPPSVSAAPGQKVTMSCSGSSSNIGNNYVSWYQQLPGTAPKLLIYDN | 23 |
| | NKRPSGIPDRFSGSKSGTSATLGITGLQTGDEADYYCGTWDSSLGAVVFGGGT | |
| | KVTVL | |
| 3 | QSVVTQPASVSGSPGQSITISCTGTSSDVGGYNYVSWYQQHPGKAPKLVIYEV | 24 |
| | SNRPSGVSNRFSGSKSGNTASLTISGLQAEDEADYYCSSYTSSSTLVFGGGTKLT | |
| | VL | |

TABLE 7
HCDRS in Chothia Numbering Scheme

| CD19 | H-CDR1 | | H-CDR2 | | H-CDR3 | |
|---|---|---|---|---|---|---|
| Binder | Sequence | SEQ ID NO: | Sequence | SEQ ID NO: | Sequence | SEQ ID NO: |
| 1 | GFTVSSN | 195 | YSGGS | 198 | ARDGGLDTLYGMDV | 201 |
| 2 | GFTFSDY | 196 | SSSGST | 199 | ARVEGWSLLDY | 202 |
| 3 | GYSFTSY | 197 | YPGDSD | 200 | ARLPFVGATTGGFDY | 203 |

TABLE 8
LCDRS in Chothia Numbering Scheme

| CD19 | L-CDR1 | | L-CDR2 | | L-CDR3 | |
|---|---|---|---|---|---|---|
| Binder | Sequence | SEQ ID NO: | Sequence | SEQ ID NO: | Sequence | SEQ ID NO: |
| 1 | TGSSSNIGAGYDVH | 204 | KLLIYGNSNRPS | 207 | QSYDSSLSGSYV | 210 |
| 2 | SGSSSNIGNNYVS | 205 | KLLIYDNNKRPS | 208 | GTWDSSLGAVV | 211 |
| 3 | TGTSSDVGGYNYVS | 206 | KLVIYEVSNRPS | 209 | SSYTSSSTLV | 212 |

TABLE 9
HCDRS in IMGT Numbering Scheme

| CD19 | H-CDR1 | | H-CDR2 | | H-CDR3 | |
|---|---|---|---|---|---|---|
| Binder | Sequence | SEQ ID NO: | Sequence | SEQ ID NO: | Sequence | SEQ ID NO: |
| 1 | GFTVSSNY | 213 | IYSGGST | 216 | ARDGGLDTLYGMDV | 201 |
| 2 | GFTFSDYY | 214 | SSSGSTT | 217 | ARVEGWSLLDY | 202 |
| 3 | GYSFTSYW | 215 | IYPGDSDT | 218 | ARLPFVGATTGGFDY | 203 |

TABLE 10
LCDRS in IMGT Numbering Scheme

| CD19 | L-CDR1 | | L-CDR2 | | L-CDR3 | |
|---|---|---|---|---|---|---|
| Binder | Sequence | SEQ ID NO: | Sequence | SEQ ID NO: | Sequence | SEQ ID NO: |
| 1 | SSNIGAGYD | 219 | GNS | | QSYDSSLSGSYV | 210 |
| 2 | SSNIGNNY | 220 | DNN | | GTWDSSLGAVV | 211 |
| 3 | SSDVGGYNY | 221 | EVS | | SSYTSSSTLV | 212 |

TABLE 11
Full CD19 Binder scFv Sequences

| CD19 | | |
|---|---|---|
| Binder | | SEQ ID NO: |
| 1 (VH-VL) | EVQLVESGGGLIQPGGSLRLSCAASGFTVSSNYMSWVRQAPGKGLEWVSVI | 25 |
| | YSGGSTYYADSVKGRFTISRDNSKNTLYLQMNSLRAEDTAVYYCARDGGLDT | |
| | LYGMDVWGQGTTVTVSSGGGGSGGGGSGGGGSQSVVTQPPSVSGAPGQ | |
| | RVTISCTGSSSNIGAGYDVHWYQQLPGTAPKLLIYGNSNRPSGVPDRFSGSK | |
| | SGTSASLAITGLQAEDEADYYCQSYDSSLSGSYVFGTGTKVTVL | |
| 2 (VH-VL) | EVHLVESGGGLVQPGGSLRLSCAASGFTFSDYYMSWIRQAPGKGLEWVSYI | 26 |
| | SSSGSTTYYADSVKGRFTISRDNAKNSLYLQMNSLRAEDTAVYYCARVEGWS | |
| | LLDYWGQGTLVTVSSGGGGSGGGGSGGGGSQSVLTQPPSVSAAPGQKVT | |
| | MSCSGSSSNIGNNYVSWYQQLPGTAPKLLIYDNNKRPSGIPDRFSGSKSGTS | |
| | ATLGITGLQTGDEADYYCGTWDSSLGAVVFGGGTKVTVL | |
| 3 (VH-VL) | EVQLVQSGAEVKKPGESLKISCKGSGYSFTSYWIGWVRQMPGKGLEWMGII | 27 |
| | YPGDSDTRYSPSFQGQVTISADKSISTAYLOWSSLKASDTAMYYCARLPFVG | |
| | ATTGGFDYWGQGTLVTVSSGGGGSGGGGSGGGGSQSVVTQPASVSGSPG | |
| | QSITISCTGTSSDVGGYNYVSWYQQHPGKAPKLVIYEVSNRPSGVSNRFSGS | |
| | KSGNTÅSLTISGLQAEDEADYYCSSYTSSSTLVFGGGTKLTVL | |
| 1 (VL-VH) | QSVVTQPPSVSGÅPGQRVTISCTGSSSNIGAGYDVHWYQQLPGTAPKLLIYG | 228 |
| | NSNRPSGVPDRFSGSKSGTSASLAITGLQAEDEADYYCQSYDSSLSGSYVFGT | |
| | GTKVTVLGGGGSGGGGSGGGGSEVQLVESGGGLIQPGGSLRLSCAASGFTV | |
| | SSNYMSWVRQAPGKGLEWVSVIYSGGSTYYADSVKGRFTISRDNSKNTLYL | |
| | QMNSLRAEDTAVYYCARDGGLDTLYGMDVWGQGTTVTVSS | |
| 2 (VL-VH) | QSVLTQPPSVSAAPGQKVTMSCSGSSSNIGNNYVSWYQQLPGTAPKLLIYD | 229 |
| | NNKRPSGIPDRFSGSKSGTSATLGITGLQTGDEADYYCGTWDSSLGAVVFG | |
| | GGTKVTVLGGGGSGGGGSGGGGSEVHLVESGGGLVQPGGSLRLSCAASGF | |
| | TFSDYYMSWIRQAPGKGLEWVSYISSSGSTTYYADSVKGRFTISRDNAKNSL | |
| | YLOMNSLRAEDTAVYYCARVEGWSLLDYWGQGTLVTVSS | |
| 3 (VL-VH) | QSVVTQPASVSGSPGQSITISCTGTSSDVGGYNYVSWYQQHPGKAPKLVIYE | 230 |
| | VSNRPSGVSNRFSGSKSGNTASLTISGLQAEDEADYYCSSYTSSSTLVFGGGT | |
| | KLTVLGGGGSGGGGSGGGGSEVQLVQSGAEVKKPGESLKISCKGSGYSFTSY | |
| | WIGWVRQMPGKGLEWMGIIYPGDSDTRYSPSFQGQVTISADKSISTAYLQ | |
| | WSSLKASDTAMYYCARLPFVGATTGGFDYWGQGTLVTVSS | |

TABLE 12
Exemplary sequences of signal peptides

| SEQ | | |
|---|---|---|
| ID NO: | Sequence | Description |
| 28 | MALPVTALLLP | CD8α signal peptide |
| | LALLLHAARP | |
| 29 | METDTLLLWVL | IgK signal peptide |
| | LLWVPGSTG | |
| 30 | MLLLVTSLLLC | GMCSFR-α (CSF2RA) |
| | ELPHPAFLLIP | signal peptide |
| 31 | MEFGLSWLFLV | Immunoglobulin heavy |
| | AILKGVQCSR | chain signal peptide |

TABLE 13
Exemplary sequences of linkers

| SEQ | | |
|---|---|---|
| ID NO: | Sequence | Description |
| 32 | GGGGSGGGGSGGGGS | (G4S)3 linker |
| 33 | GSTSGSGKPGSGEGSTKG | Whitlow linker |

TABLE 14
Exemplary sequences of hinge domains

| SEQ | | |
|---|---|---|
| ID NO: | Sequence | Description |
| 34 | TTTPAPRPPTPAPTIASQPLS | CD8α hinge |
| | LRPEACRPAAGGAVHTRGLDF | domain |
| | ACD | |
| 35 | IEVMYPPPYLDNEKSNGTIIH | CD28 hinge |
| | VKGKHLCPSPLFPGPSKP | domain |
| 36 | AAAIEVMYPPPYLDNEKSNGT | CD28 hinge |
| | IIHVKGKHLCPSPLFPGPSKP | domain |
| 37 | ESKYGPPCPPCP | IgG4 hinge |
| | | domain |
| 38 | ESKYGPPCPSCP | IgG4 hinge |
| | | domain |
| 39 | ESKYGPPCPPCPAPEFLGGPS | IgG4 hinge- |
| | VFLFPPKPKDTLMISRTPEVT | CH2-CH3 |
| | CVVVDVSQEDPEVQFNWYVDG | domain |
| | VEVHNAKTKPREEQFNSTYRV | |
| | VSVLTVLHQDWLNGKEYKCKV | |
| | SNKGLPSSIEKTISKAKGQPR | |
| | EPQVYTLPPSQEEMTKNQVSL | |
| | TCLVKGFYPSDIAVEWESNGQ | |
| | PENNYKTTPPVLDSDGSFFLY | |
| | SRLTVDKSRWQEGNVFSCSVM | |
| | HEALHNHYTQKSLSLSLGK | |

TABLE 15
Exemplary sequences of transmembrane domains

| SEQ | | |
|---|---|---|
| ID NO: | Sequence | Description |
| 40 | IYIWAPLAGTCGVL | CD8α transmembrane |
| | LLSLVITLYC | domain |
| 41 | FWVLVVVGGVLACY | CD28 transmembrane |
| | SLLVTVAFIIFWV | domain |
| 42 | MFWVLVVVGGVLAC | CD28 transmembrane |
| | YSLLVTVAFIIFWV | domain |

TABLE 16
Exemplary sequences of intracellular
costimulatory and/or signaling domains

| SEQ | | |
|---|---|---|
| ID NO: | Sequence | Description |
| 43 | KRGRKKLLYIFKQPFMRPV | 4-1BB |
| | QTTQEEDGCSCRFPEEEEG | costimulatory |
| | GCEL | domain |
| 44 | RSKRSRLLHSDYMNMTPRR | CD28 |
| | PGPTRKHYQPYAPPRDFAA | costimulatory |
| | YRS | domain |
| 45 | RSKRSRGGHSDYMNMTPRR | CD28 |
| | PGPTRKHYQPYAPPRDFAA | costimulatory |
| | YRS | domain |
| | | (LL > GG mutant) |
| 46 | RVKFSRSADAPAYQQGQNQ | CD3ζ signaling |
| | LYNELNLGRREEYDVLDKR | domain |
| | RGRDPEMGGKPRRKNPQEG | |
| | LYNELQKDKMAEAYSEIGM | |
| | KGERRRGKGHDGLYQGLST | |
| | ATKDTYDALHMQALPPR | |
| 47 | RVKFSRSADAPAYKQGQNQ | CD3ζ signaling |
| | LYNELNLGRREEYDVLDKR | domain |
| | RGRDPEMGGKPRRKNPQEG | (with Q to K |
| | LYNELQKDKMAEAYSEIGM | mutation at |
| | KGERRRGKGHDGLYQGLST | position 14) |
| | ATKDTYDALHMQALPPR | |

TABLE 17
Exemplary sequences of CAR components

| | | SEQ |
|---|---|---|
| Component | Sequence | ID NO: |
| Extracellular | | |
| binding | | |
| domain | | |
| CD19 Binder 1 | EVQLVESGGGLIQPGGSLRLSCAASGFTVSSNYMSWVRQAPGK | 25 |
| (VH-VL) | GLEWVSVIYSGGSTYYADSVKGRFTISRDNSKNTLYLQMNSLR | |
| | AEDTAVYYCARDGGLDTLYGMDVWGQGTTVTVSSGGGGSGGGG | |
| | SGGGGSQSVVTQPPSVSGAPGQRVTISCTGSSSNIGAGYDVHW | |
| | YQQLPGTAPKLLIYGNSNRPSGVPDRFSGSKSGTSASLAITGL | |
| | QAEDEADYYCQSYDSSLSGSYVFGTGTKVTVL | |
| CD19 Binder 2 | EVHLVESGGGLVQPGGSLRLSCAASGFTFSDYYMSWIRQAPGK | 26 |
| (VH-VL) | GLEWVSYISSSGSTTYYADSVKGRFTISRDNAKNSLYLQMNSL | |
| | RAEDTAVYYCARVEGWSLLDYWGQGTLVTVSSGGGGSGGGGSG | |
| | GGGSQSVLTQPPSVSAAPGQKVTMSCSGSSSNIGNNYVSWYQQ | |
| | LPGTAPKLLIYDNNKRPSGIPDRFSGSKSGTSATLGITGLQTG | |
| | DEADYYCGTWDSSLGAVVFGGGTKVTVL | |
| CD19 Binder 3 | EVQLVQSGAEVKKPGESLKISCKGSGYSFTSYWIGWVRQMPGK | 27 |
| (VH-VL) | GLEWMGIIYPGDSDTRYSPSFQGQVTISADKSISTAYLQWSSL | |
| | KASDTAMYYCARLPFVGATTGGFDYWGQGTLVTVSSGGGGSGG | |
| | GGSGGGGSQSVVTQPASVSGSPGQSITISCTGTSSDVGGYNYV | |
| | SWYQQHPGKAPKLVIYEVSNRPSGVSNRFSGSKSGNTASLTIS | |
| | GLQAEDEADYYCSSYTSSSTLVFGGGTKLTVL | |
| CD19 Binder 1 | QSVVTQPPSVSGAPGQRVTISCTGSSSNIGAGYDVHWYQQLPG | 228 |
| (VL-VH) | TAPKLLIYGNSNRPSGVPDRFSGSKSGTSASLAITGLQAEDEA | |
| | DYYCQSYDSSLSGSYVFGTGTKVTVLGGGGSGGGGSGGGGSEV | |
| | QLVESGGGLIQPGGSLRLSCAASGFTVSSNYMSWVRQAPGKGL | |
| | EWVSVIYSGGSTYYADSVKGRFTISRDNSKNTLYLQMNSLRAE | |
| | DTAVYYCARDGGLDTLYGMDVWGQGTTVTVSS | |
| CD19 Binder 2 | QSVLTQPPSVSAAPGQKVTMSCSGSSSNIGNNYVSWYQQLPGT | 229 |
| (VL-VH) | APKLLIYDNNKRPSGIPDRFSGSKSGTSATLGITGLQTGDEAD | |
| | YYCGTWDSSLGAVVFGGGTKVTVLGGGGSGGGGSGGGGSEVHL | |
| | VESGGGLVQPGGSLRLSCAASGFTFSDYYMSWIRQAPGKGLEW | |
| | VSYISSSGSTTYYADSVKGRFTISRDNAKNSLYLQMNSLRAED | |
| | TAVYYCARVEGWSLLDYWGQGTLVTVSS | |
| CD19 Binder 3 | QSVVTQPASVSGSPGQSITISCTGTSSDVGGYNYVSWYQQHPG | 230 |
| (VL-VH) | KAPKLVIYEVSNRPSGVSNRFSGSKSGNTASLTISGLQAEDEA | |
| | DYYCSSYTSSSTLVFGGGTKLTVLGGGGSGGGGSGGGGSEVQL | |
| | VQSGAEVKKPGESLKISCKGSGYSFTSYWIGWVRQMPGKGLEW | |
| | MGIIYPGDSDTRYSPSFQGQVTISADKSISTAYLQWSSLKASD | |
| | TAMYYCARLPFVGATTGGFDYWGQGTLVTVSS | |
| Spacer | | |
| (e.g., hinge) | | |
| IgG4 Hinge | ESKYGPPCPPCP | 37 |
| CD8 Hinge | TTTPAPRPPTPAPTIASQPLSLRPE | 48 |
| CD28 | IEVMYPPPYLDNEKSNGTIIHVKGKHLCPSPLFPGPSKP | 35 |
| Transmembrane | | |
| CD8 | ACRPAAGGAVHTRGLDFACDIYIWAPLAGTCGVLLLSLVITLY | 49 |
| | C | |
| CD28 | FWVLVVVGGVLACYSLLVTVAFIIFWV | 41 |
| CD28 | MFWVLVVVGGVLACYSLLVTVAFIIFWV | 42 |
| Costimulatory | | |
| domain | | |
| CD28 | RSKRSRLLHSDYMNMTPRRPGPTRKHYQPYAPPRDFAAYRS | 44 |
| 4-1BB | KRGRKKLLYIFKQPFMRPVQTTQEEDGCSCRFPEEEEGGCEL | 43 |
| Primary | | |
| Signaling | | |
| Domain | | |
| CD3zeta | RVKFSRSADAPAYQQGQNQLYNELNLGRREEYDVLDKRRGRDP | 46 |
| | EMGGKPRRKNPQEGLYNELQKDKMAEAYSEIGMKGERRRGKGH | |
| | DGLYQGLSTATKDTYDALHMQALPPR | |
| CD3zeta | RVKFSRSADAPAYKQGQNQLYNELNLGRREEYDVLDKRRGRDP | 47 |
| (Q > K) | EMGGKPRRKNPQEGLYNELQKDKMAEAYSEIGMKGERRRGKGH | |
| | DGLYQGLSTATKDTYDALHMQALPPR | |

TABLE 18
HCDRS In Kabat Numbering Scheme

| | H-CDR1 | | H-CDR2 | | H-CDR3 | |
|---|---|---|---|---|---|---|
| | | SEQ | | SEQ | | SEQ |
| CD4 | | ID | | ID | | ID |
| Binder | Sequence | NO: | Sequence | NO: | Sequence | NO: |
| 1 | SYWIE | 50 | EILPGSGST | 54 | RGYGYDEG | 58 |
| | | | SYNEKFKG | | FDY | |
| 2 | DYVIS | 51 | EIYPGSGSS | 55 | PGDLGFAY | 59 |
| | | | YYNEKFKG | | | |
| 3 | THWMH | 52 | MINPSDGVT | 56 | EYYGEGFD | 60 |
| | | | YYAQTFQG | | Y | |
| 4 | GYWMY | 53 | AISPGGGST | 57 | SLTATHTY | 61 |
| | | | YYPDSVKG | | EYDY | |

TABLE 19
LCDRS in Kabat Numbering Scheme

| | L-CDR1 | | L-CDR2 | | L-CDR3 | |
|---|---|---|---|---|---|---|
| | | SEQ | | SEQ | | SEQ |
| CD4 | | ID | | ID | | ID |
| Binder | Sequence | NO: | Sequence | NO: | Sequence | NO: |
| 1 | ASQDINSY | 62 | RANRLVD | 65 | LQYDEFPPT | 68 |
| | LS | | | | | |
| 2 | ASQSVDYD | 63 | AASNLES | 66 | QQSNKDPFT | 69 |
| | GDSYMN | | | | | |
| 3 | RASQGISN | 64 | SASNLQS | 67 | QQSYSTPLT | 70 |
| | YLA | | | | | |

TABLE 20
VH Sequences

| CD4 | | SEQ |
|---|---|---|
| Binder | VH Sequence | ID NO: |
| 1 | QVQLQQSGAELMKPGASVKISCKATGYTFSS | 71 |
| | YWIEWVKQRPGHGLEWIGEILPGSGSTSYNE | |
| | KFKGKATFTADTSSNTAYMQLSSLTSEDSAV | |
| | YYCARRGYGYDEGFDYWGQGTTLTVSS | |
| 2 | QVQLQQSGPELVKPGASVKMSCKASGYTFTD | 72 |
| | YVISWVRQAPGQGLEWIGEIYPGSGSSYYNE | |
| | KFKGRATLTADKSSNTAYMQLSSLRSEDSAV | |
| | YFCARPGDLGFAYWGQGTLVTVSS | |
| 3 | QVQLVQSGAEVKKPGASVKVSCKASGYSLIT | 73 |
| | HWMHWVRQAPGQGLEWMGMINPSDGVTYYAQ | |
| | TFQGRVTMTRDTSTSTVYMELSSLRSEDTAV | |
| | YYCAREYYGEGFDYWGQGTLVTVSS | |
| 4 | EVQLVESGGGLVQSGGSLRLSCAASGFTFSG | 74 |
| | YWMYWVRQAPGKGLEWVSAISPGGGSTYYPD | |
| | SVKGRFTISRDNAKNTLYLQMNSLEPEDTAL | |
| | YYCASSLTATHTYEYDYWGQGTQVTVSS | |

TABLE 21
VL Sequences

| CD4 | | SEQ |
|---|---|---|
| Binder | VH Sequence | ID NO: |
| 1 | DIKMTQSPSSMYASLGERVTITCKASQDINS | 75 |
| | YLSWFQQKPGKSPKTLIYRANRLVDGVPSRF | |
| | SGSGSGQDYSLTISSLEYEDMGIYYCLQYDE | |
| | FPPTFGAGTKLELKR | |
| 2 | DIVLTQSPSSLAVSLGQRATISCKASQSVDY | 76 |
| | DGDSYMNWYQQKPGQPPKLLIYAASNLESGI | |
| | PARFSGSGSGTDFTLTIHPVEEEDAATYYCQ | |
| | QSNKDPFTFGGGTKLELKR | |
| 3 | DIQMTQSPSSLSASVGDRVTITCRASQGISN | 77 |
| | YLAWYQQKPGKAPKLLIYSASNLQSGVPSRF | |
| | SGSGSGTDFTLTISSLQPEDFATYYCQQSYS | |
| | TPLTFGGGTKVEIKR* | |

TABLE 22
HCDRS in Kabat Numbering Scheme

| | H-CDR1 | | H-CDR2 | | H-CDR3 | |
|---|---|---|---|---|---|---|
| | | SEQ | | SEQ | | SEQ |
| CD8 | | ID | | ID | | ID |
| Binder | Sequence | NO: | Sequence | NO: | Sequence | NO: |
| 1 | SYAIS | 78 | IIDPSDGNT | 82 | ERAAAGYY | 86 |
| | | | NYAQNFQG | | YYMDV | |
| 2 | DYYIQ | 79 | WINPNSGGT | 83 | EGDYYYGM | 87 |
| | | | SYAQKFQG | | DA | |
| 3 | SYYMH | 80 | GFDPEDGET | 84 | DQGWGMDV | 88 |
| | | | IYAQKFQG | | | |
| 4 | NHYMH | 81 | WMNPNSGNT | 85 | SESGSDLD | 89 |
| | | | GYAQKFQG | | Y | |

TABLE 23
LCDRS in Kabat Numbering Scheme

| | L-CDR1 | | L-CDR2 | | L-CDR3 | |
|---|---|---|---|---|---|---|
| | | SEQ | | SEQ | | SEQ |
| CD8 | | ID | | ID | | ID |
| Binder | Sequence | NO: | Sequence | NO: | Sequence | NO: |
| 1 | RASQSISS | 90 | AASSLQS | 94 | QQSYSTPLT | 98 |
| | YLN | | | | | |
| 2 | RSSQSLLH | 91 | LGSNRAS | 95 | MQGLQTPHT | 99 |
| | SNGYNYLD | | | | | |
| 3 | RASQSISS | 92 | AASSLQS | 96 | QQTYSTPYT | 100 |
| | YLN | | | | | |
| 4 | RASQTIGN | 93 | GASNLHT | 97 | QQTYSAPLT | 101 |
| | YVN | | | | | |

TABLE 24
VH Sequences

| | | SEQ |
|---|---|---|
| CD8 | | ID |
| Binder | VH Sequence | NO: |
| 1 | QVQLVQSGAEVKKPGASVKVSCKASGGTFSSY | 102 |
| | AISWVRQAPGQGLEWMGIIDPSDGNTNYAQNF | |
| | QGRVTMTRDTSTSTVYMELSSLRSEDTAVYYC | |
| | AKERAAAGYYYYMDVWGQGTTVTVSS | |
| 2 | QVQLVQSGAEVKKPGASVKVSCKASGYTFTDY | 103 |
| | YIQWVRQAPGQGLEWMGWINPNSGGTSYAQKF | |
| | QGRVTMTRDTSTSTVYMELSSLRSEDTAVYYC | |
| | AKEGDYYYGMDAWGQGTMVTVSS | |
| 3 | QVQLVQSGAEVKKPGASVKVSCKASGYTFTSY | 104 |
| | YMHWVRQAPGQGLEWMGGFDPEDGETIYAQKF | |
| | QGRVTMTRDTSTSTVYMELSSLRSEDTAVYYC | |
| | ARDQGWGMDVWGQGTTVTVSS | |
| 4 | QVQLVQSGAEVKKPGASVKVSCKASGYTFTNH | 105 |
| | YMHWVRQAPGQGLEWMGWMNPNSGNTGYAQKF | |
| | QGRVTMTRDTSTSTVYMELSSLRSEDTAVYYC | |
| | ASSESGSDLDYWGQGTLVTVSS | |

TABLE 25
VL Sequences

| | | SEQ |
|---|---|---|
| CD8 | | ID |
| Binder | VH Sequence | NO: |
| 1 | DIQMTQSPSSLSASVGDRVTITCRASQSISSY | 106 |
| | LNWYQQKPGKAPKLLIYAASSLQSGVPSRFSG | |
| | SGSGTDFTLTISSLQPEDFATYYCQQSYSTPL | |
| | TFGGGTKVEIKR | |
| 2 | DIVMTQSPLSLPVTPGEPASISCRSSQSLLHS | 107 |
| | NGYNYLDWYLQKPGQSPQLLIYLGSNRASGVP | |
| | DRFSGSGSGTDFTLKISRVEAEDVGVYYCMQG | |
| | LQTPHTFGQGTKVEIKR | |
| 3 | DIQMTQSPSSLSASVGDRVTITCRASQSISSY | 108 |
| | LNWYQQKPGKAPKLLIYAASSLQSGVPSRFSG | |
| | SGSGTDFTLTISSLQPEDFATYYCQQTYSTPY | |
| | TFGQGTKLEIKR | |
| 4 | DIQMTQSPSSLSASVGDRVTITCRASQTIGNY | 109 |
| | VNWYQQKPGKAPKLLIYGASNLHTGVPSRFSG | |
| | SGSGTDFTLTISSLQPEDFATYYCQQTYSAPL | |
| | TFGGGTKVEIKR | |

TABLE 26
Exemplary G and F Protein Sequences

| SEQ | | |
|---|---|---|
| ID | | |
| NO: | SEQUENCE | ANNOTATION |
| 110 | MVVILDKRCY CNLLILILMI SECSVGILHY EKLSKIGLVK | Nipah virus |
| | GVTRKYKIKS NPLTKDIVIK MIPNVSNMSQ CTGSVMENYK | NiV-F with |
| | TRLNGILTPI KGALEIYKNN THDLVGDVRL AGVIMAGVAI | signal sequence |
| | GIATAAQITA GVALYEAMKN ADNINKLKSS IESTNEAVVK | (aa 1-546) |
| | LQETAEKTVY VLTALQDYIN TNLVPTIDKI SCKQTELSLD | Uniprot Q9IH63 |
| | LALSKYLSDL LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK | |
| | KRNTYSRLED RRVRPTSSGD LYYIGT | |
| 111 | ILHY EKLSKIGLVK GVTRKYKIKS NPLTKDIVIK MIPNVSNMSQ | Nipah virus |
| | CTGSVMENYK TRLNGILTPI KGALEIYKNN THDLVGDVRL | NiV-F F0 |
| | AGVIMAGVAI GIATAAQITA GVALYEAMKN ADNINKLKSS | (aa 27-546) |
| | IESTNEAVVK LQETAEKTVY VLTALQDYIN TNLVPTIDKI | |
| | SCKQTELSLD LALSKYLSDL LFVFGPNLQD PVSNSMTIQA | |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRNTYSRLED RRVRPTSSGD LYYIGT | |
| 112 | ILHYEKLSKIGLVKGVTRKYKIKSNPLTKDIVIKMIPNVSNMSQC | Nipah virus |
| | TGSVMENYKTRLNGILTPIKGALEIYKNNTHDLVGDVR | NiV-F F2 |
| | | (aa 27-109) |
| 113 | LAGVIMAGVAIGIATAAQITAGVALYEAMKNADNINKLKSSIES | Nipah virus |
| | TNEAVVKLQETAEKTVYVLTALQDYINTNLVPTIDKISCKQTELS | NiV F F1 |
| | LDLALSKYLSDLLFVFGPNLQDPVSNSMTIQAISQAFGGNYETL | (aa 110-546) |
| | LRTLGYATEDFDDLLESDSITGQIIYVDLSSYYIIVRVYFPILTEIQQ | |
| | AYIQELLPVSFNNDNSEWISIVPNFILVRNTLISNIEIGFCLITKRS | |
| | VICNQDYATPMTNNMRECLTGSTEKCPRELVVSSHVPRFALSN | |
| | GVLFANCISVTCQCQTTGRAISQSGEQTLLMIDNTTCPTAVLG | |
| | NVIISLGKYLGSVNYNSEGIAIGPPVFTDKVDISSQISSMNQSLQ | |
| | QSKDYIKEAQRLLDTVNPSLISMLSMIILYVLSIASLCIGLITFISFII | |
| | VEKKRNTYSRLEDRRVRPTSSGDLYYIGT | |
| 114 | ILHY EKLSKIGLVK GVTRKYKIKS NPLTKDIVIK MIPNVSNMSQ | Nipah virus |
| | CTGSVMENYK TRLNGILTPI KGALEIYKNN THDLVGDVRL | NiV-F F0 |
| | AGVIMAGVAI GIATAAQITA GVALYEAMKN ADNINKLKSS | truncation |
| | IESTNEAVVK LQETAEKTVY VLTALQDYIN TNLVPTIDKI | (aa 525-544) |
| | SCKQTELSLD LALSKYLSDL LFVFGPNLQD PVSNSMTIQA | |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRNTGT | |
| 115 | LAGVIMAGVAIGIATAAQITAGVALYEAMKNADNINKLKSSIES | Nipah virus |
| | TNEAVVKLQETAEKTVYVLTALQDYINTNLVPTIDKISCKQTELS | NiV F F1 |
| | LDLALSKYLSDLLFVFGPNLQDPVSNSMTIQAISQAFGGNYETL | (aa 110-546) |
| | LRTLGYATEDFDDLLESDSITGQIIYVDLSSYYIIVRVYFPILTEIQQ | truncation |
| | AYIQELLPVSFNNDNSEWISIVPNFILVRNTLISNIEIGFCLITKRS | (aa 525-544) |
| | VICNQDYATPMTNNMRECLTGSTEKCPRELVVSSHVPRFALSN | |
| | GVLFANCISVTCQCQTTGRAISQSGEQTLLMIDNTTCPTAVLG | |
| | NVIISLGKYLGSVNYNSEGIAIGPPVFTDKVDISSQISSMNQSLQ | |
| | QSKDYIKEAQRLLDTVNPSLISMLSMIILYVLSIASLCIGLITFISFII | |
| | VEKKRNTGT | |
| 116 | ILHY EKLSKIGLVK GVTRKYKIKS NPLTKDIVIK MIPNVSNMSQ | Nipah virus NiV-F F0 |
| | CTGSVMENYK TRLNGILTPI KGALEIYKNQ THDLVGDVRL | truncation (aa 525- |
| | AGVIMAGVAI GIATAAQITA GVALYEAMKN ADNINKLKSS | 544) AND mutation on |
| | IESTNEAVVK LQETAEKTVY VLTALQDYIN TNLVPTIDKI | N-linked glycosylation |
| | SCKQTELSLD LALSKYLSDL LFVFGPNLQD PVSNSMTIQA | site |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRNTGT | |
| 117 | MVVILDKRCY CNLLILILMI SECSVGILHY EKLSKIGLVK | Truncated NiV fusion |
| | GVTRKYKIKS NPLTKDIVIK MIPNVSNMSQ CTGSVMENYK | glycoprotein |
| | TRLNGILTPI KGALEIYKNN THDLVGDVRL AGVIMAGVAI | (FcDelta22) at |
| | GIATAAQITA GVALYEAMKN ADNINKLKSS IESTNEAVVK | cytoplasmic tail |
| | LQETAEKTVY VLTALQDYIN TNLVPTIDKI SCKQTELSLD | (with signal sequence) |
| | LALSKYLSDL LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNT | |
| 118 | ILHY EKLSKIGLVK GVTRKYKIKS NPLTKDIVIK | Truncated NiV fusion |
| | MIPNVSNMSQ CTGSVMENYK TRLNGILTPI KGALEIYKNN | glycoprotein |
| | THDLVGDVRL AGVIMAGVAI GIATAAQITA GVALYEAMKN | (FcDelta22) F0 |
| | ADNINKLKSS IESTNEAVVK LQETAEKTVY VLTALQDYIN | |
| | TNLVPTIDKI SCKQTELSLD LALSKYLSDL LFVFGPNLQD | |
| | PVSNSMTIQA ISQAFGGNYE TLLRTLGYAT EDFDDLLESD | |
| | SITGQIIYVD LSSYYIIVRV YFPILTEIQQ AYIQELLPVS | |
| | FNNDNSEWIS IVPNFILVRN TLISNIEIGF CLITKRSVIC | |
| | NQDYATPMTN NMRECLTGST EKCPRELVVS SHVPRFALSN | |
| | GVLFANCISV TCQCQTTGRA ISQSGEQTLL MIDNTTCPTA | |
| | VLGNVIISLG KYLGSVNYNS EGIAIGPPVF TDKVDISSQI | |
| | SSMNQSLQQS KDYIKEAQRL LDTVNPSLIS MLSMIILYVL | |
| | SIASLCIGLI TFISFIIVEK KRNT | |
| 119 | LAGVIMAGVAI GIATAAQITA GVALYEAMKN ADNINKLKSS | Truncated NiV fusion |
| | IESTNEAVVK LQETAEKTVY VLTALQDYIN TNLVPTIDKI | glycoprotein |
| | SCKQTELSLD LALSKYLSDL LFVFGPNLQD PVSNSMTIQA | (FcDelta22) F1 |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRNT | |
| 120 | MGPAENKKVR FENTTSDKGK IPSKVIKSYY GTMDIKKINE | NiVG protein |
| | GLLDSKILSA FNTVIALLGS IVIIVMNIMI IQNYTRSTDN | attachment |
| | QAVIKDALQG IQQQIKGLAD KIGTEIGPKV SLIDTSSTIT | glycoprotein (602 aa) |
| | IPANIGLLGS KISQSTASIN ENVNEKCKFT LPPLKIHECN | |
| | ISCPNPLPFR EYRPQTEGVS NLVGLPNNIC LQKTSNQILK | |
| | PKLISYTLPV VGQSGTCITD PLLAMDEGYF AYSHLERIGS | |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRFNTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QC | |
| 121 | MGKVR FENTTSDKGK IPSKVIKSYY GTMDIKKINE | NiVG protein |
| | GLLDSKILSA FNTVIALLGS IVIIVMNIMI IQNYTRSTDN | attachment |
| | QAVIKDALQG IQQQIKGLAD KIGTEIGPKV SLIDTSSTIT | glycoprotein |
| | IPANIGLLGS KISQSTASIN ENVNEKCKFT LPPLKIHECN | Truncated Δ5 |
| | ISCPNPLPFR EYRPQTEGVS NLVGLPNNIC LQKTSNQILK | |
| | PKLISYTLPV VGQSGTCITD PLLAMDEGYF AYSHLERIGS | |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRFNTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QC | |
| 122 | MGNTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS | glycoprotein |
| | KISQSTASIN ENVNEKCKFT LPPLKIHECN ISCPNPLPFR | Truncated Δ10 |
| | EYRPQTEGVS NLVGLPNNIC LQKTSNQILK PKLISYTLPV | |
| | VGQSGTCITD PLLAMDEGYF AYSHLERIGS CSRGVSKQRI | |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 123 | MGKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS | glycoprotein |
| | KISQSTASIN ENVNEKCKFT LPPLKIHECN ISCPNPLPFR | Truncated Δ15 |
| | EYRPQTEGVS NLVGLPNNIC LQKTSNQILK PKLISYTLPV | |
| | VGQSGTCITD PLLAMDEGYF AYSHLERIGS CSRGVSKQRI | |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 124 | MGSKVIKSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ20 |
| | NLVGLPNNIC LQKTSNQILK PKLISYTLPV VGQSGTCITD | |
| | PLLAMDEGYF AYSHLERIGS CSRGVSKQRI IGVGEVLDRG | |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 125 | MGSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ25 |
| | NLVGLPNNIC LQKTSNQILK PKLISYTLPV VGQSGTCITD | |
| | PLLAMDEGYF AYSHLERIGS CSRGVSKQRI IGVGEVLDRG | |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 126 | MGTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ30 |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST VGDPILNSTY | |
| | WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 127 | MKKINEGLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated and |
| | LQKTSNQILK PKLISYTLPV VGQSGTCITD PLLAMDEGYF | mutated |
| | AYSHLERIGS CSRGVSKQRI IGVGEVLDRG DEVPSLFMTN | (E501A, W504A, |
| | VWTPPNPNTV YHCSAVYNNE FYYVLCAVST VGDPILNSTY | Q530A, E533A) NIV G |
| | WSGSLMMTRL AVKPKSNGGG YNQHQLALRS | protein (Gc Δ 34) |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PAICAEGVYN DAFLIDRINW ISAGVFLDSN ATAANPVFTV | |
| | FKDNEILYRA QLASEDTNAQ KTITNCFLLK NKIWCISLVE | |
| | IYDTGDNVIR PKLFAVKIPE QCT | |
| 128 | MATQEVRLKC LLCGIIVLVL SLEGLGILHY EKLSKIGLVK | Hendra virus F protein |
| | GITRKYKIKS NPLTKDIVIK MIPNVSNVSK CTGTVMENYK | Uniprot O89342 (with |
| | SRLTGILSPI KGAIELYNNN THDLVGDVKL AGVVMAGIAI | signal sequence) |
| | GIATAAQITA GVALYEAMKN ADNINKLKSS IESTNEAVVK | |
| | LQETAEKTVY VLTALQDYIN TNLVPTIDQI SCKQTELALD | |
| | LALSKYLSDL LFVFGPNLQD PVSNSMTIQA ISQAFGGNYE | |
| | TLLRTLGYAT EDFDDLLESD SIAGQIVYVD LSSYYIIVRV | |
| | YFPILTEIQQ AYVQELLPVS FNNDNSEWIS IVPNFVLIRN | |
| | TLISNIEVKY CLITKKSVIC NQDYATPMTA SVRECLTGST | |
| | DKCPRELVVS SHVPRFALSG GVLFANCISV TCQCQTTGRA | |
| | ISQSGEQTLL MIDNTTCTTV VLGNIIISLG KYLGSINYNS | |
| | ESIAVGPPVY TDKVDISSQI SSMNQSLQQS KDYIKEAQKI | |
| | LDTVNPSLIS MLSMIILYVL SIAALCIGLI TFISFVIVEK | |
| | KRGNYSRLDD RQVRPVSNGD LYYIGT | |
| 129 | MMADSKLVSL NNNLSGKIKD QGKVIKNYYG TMDIKKINDG | Hendra virus G protein |
| | LLDSKILGAF NTVIALLGSI IIIVMNIMII QNYTRTTDNQ | Uniprot O89343 |
| | ALIKESLQSV QQQIKALTDK IGTEIGPKVS LIDTSSTITI | |
| | PANIGLLGSK ISQSTSSINE NVNDKCKFTL PPLKIHECNI | |
| | SCPNPLPFRE YRPISQGVSD LVGLPNQICL QKTTSTILKP | |
| | RLISYTLPIN TREGVCITDP LLAVQNGFFA YSHLEKIGSC | |
| | TRGIAKQRII GVGEVLDRGD KVPSMFMTNV WTPPNPSTIH | |
| | HCSSTYHEDF YYTLCAVSHV GDPILNSTSW TESLSLIRLA | |
| | VRPKSDSGDY NQKYIAITKV ERGKYDKVMP YGPSGIKQGD | |
| | TLYFPAVGFL PRTEFQYNDS NCPIIHCKYS KAENCRLSMG | |
| | VNSKSHYILR SGLLKYNLSL GGDIILQFIE IADNRLTIGS | |
| | PSKIYNSLGQ PVFYQASYSW DTMIKLGDVD TVDPLRVQWR | |
| | NNSVISRPGQ SQCPRFNVCP EVCWEGTYND AFLIDRLNWV | |
| | SAGVYLNSNQ TAENPVFAVF KDNEILYQVP LAEDDTNAQK | |
| | TITDCFLLEN VIWCISLVEI YDTGDSVIRP KLFAVKIPAQ CSES | |
| 130 | MVVILDKRCY CNLLILILMI SECSVGILHY EKLSKIGLVK | Nipah virus NiV-F F0 |
| | GVTRKYKIKS NPLTKDIVIK MIPNVSNMSQ CTGSVMENYK | truncation (aa 525- |
| | TRLNGILTPI KGALEIYKNN THDLVGDVRL AGVIMAGVAI | 544) (with signal |
| | GIATAAQITA GVALYEAMKN ADNINKLKSS IESTNEAVVK | sequence) |
| | LQETAEKTVY VLTALQDYIN TNLVPTIDKI SCKQTELSLD | |
| | LALSKYLSDL LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNTGT | |
| 131 | MVVILDKRCY CNLLILILMI SECSVGILHY EKLSKIGLVK | Nipah virus NiV-F F0 |
| | GVTRKYKIKS NPLTKDIVIK MIPNVSNMSQ CTGSVMENYK | truncation (aa 525- |
| | TRLNGILTPI KGALEIYKNQ THDLVGDVRL AGVIMAGVAI | 544) AND mutation on |
| | GIATAAQITA GVALYEAMKN ADNINKLKSS IESTNEAVVK | N-linked glycosylation |
| | LQETAEKTVY VLTALQDYIN TNLVPTIDKI SCKQTELSLD | site (with signal |
| | LALSKYLSDL LFVFGPNLQD PVSNSMTIQA ISQAFGGNYE | sequence) |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNTGT | |
| 132 | MKKINEGLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated (Gc Δ 34) |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST VGDPILNSTY | |
| | WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QCT | |
| 133 | ILHY EKLSKIGLVK GVTRKYKIKS NPLTKDIVIK MIPNVSNMSQ | Truncated mature NiV |
| | CTGSVMENYK TRLNGILTPI KGALEIYKNN THDLVGDVRL | fusion glycoprotein |
| | AGVIMAGVAI GIATAAQITA GVALYEAMKN ADNINKLKSS | (FcDelta22) at |
| | IESTNEAVVK LQETAEKTVY VLTALQDYIN TNLVPTIDKI | cytoplasmic tail |
| | SCKQTELSLD LALSKYLSDL LFVFGPNLQD PVSNSMTIQA | |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRNT | |
| 134 | MSNKRTTVLIIISYTLFYLNNAAIVGFDFDKINKIGVVQGRVLNY | gb:JQ001776:6129- |
| | KIKGDPMTKDLVLKFIPNIVNITECVREPLSRYNETVRRLLLPIHN | 8166\|Organism:Cedar |
| | MLGLYLNNTNAKMTGLMIAGVIMGGIAIGIATAAQITAGFALY | virus\|Strain |
| | EAKKNTENIQKLTDSIMKTQDSIDKLTDSVGTSILILNKLQTYIN | Name:CG1a\|Protein |
| | NQLVPNLELLSCRQNKIEFDLMLTKYLVDLMTVIGPNINNPVN | Name:fusion |
| | KDMTIQSLSLLFDGNYDIMMSELGYTPQDFLDLIESKSITGQIIY | glycoprotein\|Gene |
| | VDMENLYVVIRTYLPTLIEVPDAQIYEFNKITMSSNGGEYLSTIP | Symbol:F (with signal |
| | NFILIRGNYMSNIDVATCYMTKASVICNQDYSLPMSQNLRSCY | sequence) |
| | QGETEYCPVEAVIASHSPRFALTNGVIFANCINTICRCQDNGKTI | |
| | TQNINQFVSMIDNSTCNDVMVDKFTIKVGKYMGRKDINNINI | |
| | QIGPQIIIDKVDLSNEINKMNQSLKDSIFYLREAKRILDSVNISLIS | |
| | PSVQLFLIIISVLSFIILLIIIVYLYCKSKHSYKYNKFIDDPDYYNDYK | |
| | RERINGKASKSNNIYYVGD | |
| 135 | MALNKNMFSSLFLGYLLVYATTVQSSIHYDSLSKVGVIKGLTYN | gb:NC_025352:5950- |
| | YKIKGSPSTKLMVVKLIPNIDSVKNCTQKQYDEYKNLVRKALEP | 8712\|Organism:Mojiang |
| | VKMAIDTMLNNVKSGNNKYRFAGAIMAGVALGVATAATVTA | virus\|Strain |
| | GIALHRSNENAQAIANMKSAIQNTNEAVKQLQLANKQTLAVI | Name:Tongguan1\| |
| | DTIRGEINNNIIPVINQLSCDTIGLSVGIRLTQYYSEIITAFGPALQ | Protein Name:fusion |
| | NPVNTRITIQAISSVFNGNFDELLKIMGYTSGDLYEILHSELIRGN | protein\|Gene |
| | IIDVDVDAGYIALEIEFPNLTLVPNAVVQELMPISYNIDGDEWV | Symbol:F (with signal |
| | TLVPRFVLTRTTLLSNIDTSRCTITDSSVICDNDYALPMSHELIGC | sequence) |
| | LQGDTSKCAREKVVSSYVPKFALSDGLVYANCLNTICRCMDTD | |
| | TPISQSLGATVSLLDNKRCSVYQVGDVLISVGSYLGDGEYNAD | |
| | NVELGPPIVIDKIDIGNQLAGINQTLQEAEDYIEKSEEFLKGVNP | |
| | SIITLGSMVVLYIFMILIAIVSVIALVLSIKLTVKGNVVRQQFTYTQ | |
| | HVPSMENINYVSH | |
| 136 | MKKKTDNPTISKRGHNHSRGIKSRALLRETDNYSNGLIVENLVR | gb:NC_025256:6865- |
| | NCHHPSKNNLNYTKTQKRDSTIPYRVEERKGHYPKIKHLIDKSY | 8853\|Organism:Bat |
| | KHIKRGKRRNGHNGNIITIILLLILILKTQMSEGAIHYETLSKIGLIK | Paramyxovirus |
| | GITREYKVKGTPSSKDIVIKLIPNVTGLNKCTNISMENYKEQLDKI | Eid_hel/GH- |
| | LIPINNIIELYANSTKSAPGNARFAGVIIAGVALGVAAAAQITAGI | M74a/GHA/2009\|Strain |
| | ALHEARQNAERINLLKDSISATNNAVAELQEATGGIVNVITGM | Name:BatPV/Eid_hel/GH- |
| | QDYINTNLVPQIDKLQCSQIKTALDISLSQYYSEILTVFGPNLQN | M74a/GHA/2009\| |
| | PVTTSMSIQAISQSFGGNIDLLLNLLGYTANDLLDLLESKSITGQI | Protein Name:fusion |
| | TYINLEHYFMVIRVYYPIMTTISNAYVQELIKISFNVDGSEWVSL | protein\|Gene |
| | VPSYILIRNSYLSNIDISECLITKNSVICRHDFAMPMSYTLKECLT | Symbol:F (with signal |
| | GDTEKCPREAVVTSYVPRFAISGGVIYANCLSTTCQCYQTGKVI | sequence) |
| | AQDGSQTLMMIDNQTCSIVRIEEILISTGKYLGSQEYNTMHVS | |
| | VGNPVFTDKLDITSQISNINQSIEQSKFYLDKSKAILDKINLNLIG | |
| | SVPISILFIIAILSLILSIITFVIVMIIVRRYNKYTPLINSDPSSRRSTIQ | |
| | DVYIIPNPGEHSIRSAARSIDRDRD | |
| 137 | (GGGGGS)n wherein n is 1 to 6 | Peptide Linker |
| 138 | MPAENKKVRFENTTSDKGKIPSKVIKSYYGTMDIKKINEGLLDS | gb:AF212302\|Organism: |
| | KILSAFNTVIALLGSIVIIVMNIMIIQNYTRSTDNQAVIKDALQGI | Nipah virus\|Strain |
| | QQQIKGLADKIGTEIGPKVSLIDTSSTITIPANIGLLGSKISQSTASI | Name:UNKNOWN- |
| | NENVNEKCKFTLPPLKIHECNISCPNPLPFREYRPQTEGVSNLV | AF212302\|Protein |
| | GLPNNICLQKTSNQILKPKLISYTLPVVGQSGTCITDPLLAMDEG | Name:attachment |
| | YFAYSHLERIGSCSRGVSKQRIIGVGEVLDRGDEVPSLFMTNV | glycoprotein\|Gene |
| | WTPPNPNTVYHCSAVYNNEFYYVLCAVSTVGDPILNSTYWSG | Symbol:G |
| | SLMMTRLAVKPKSNGGGYNQHQLALRSIEKGRYDKVMPYGP | (Uniprot Q9IH62) |
| | SGIKQGDTLYFPAVGFLVRTEFKYNDSNCPITKCQYSKPENCRL | |
| | SMGIRPNSHYILRSGLLKYNLSDGENPKVVFIEISDQRLSIGSPSK | |
| | IYDSLGQPVFYQASFSWDTMIKFGDVLTVNPLVVNWRNNTVI | |
| | SRPGQSQCPRFNTCPEICWEGVYNDAFLIDRINWISAGVFLDS | |
| | NQTAENPVFTVFKDNEILYRAQLASEDTNAQKTITNCFLLKNKI | |
| | WCISLVEIYDTGDNVIRPKLFAVKIPEQCT | |
| 139 | MLSQLQKNYLDNSNQQGDKMNNPDKKLSVNFNPLELDKGQ | gb:JQ001776:8170- |
| | KDLNKSYYVKNKNYNVSNLLNESLHDIKFCIYCIFSLLIIITIINIITIS | 10275\|Organism:Cedar |
| | IVITRLKVHEENNGMESPNLQSIQDSLSSLTNMINTEITPRIGILV | virus\|Strain |
| | TATSVTLSSSINYVGTKTNQLVNELKDYITKSCGFKVPELKLHEC | Name:CG1a\|Protein |
| | NISCADPKISKSAMYSTNAYAELAGPPKIFCKSVSKDPDFRLKQI | Name:attachment |
| | DYVIPVQQDRSICMNNPLLDISDGFFTYIHYEGINSCKKSDSFKV | glycoprotein\|Gene |
| | LLSHGEIVDRGDYRPSLYLLSSHYHPYSMQVINCVPVTCNQSSF | Symbol:G |
| | VFCHISNNTKTLDNSDYSSDEYYITYFNGIDRPKTKKIPINNMTA | |
| | DNRYIHFTFSGGGGVCLGEEFIIPVTTVINTDVFTHDYCESFNCS | |
| | VQTGKSLKEICSESLRSPTNSSRYNLNGIMIISQNNMTDFKIQL | |
| | NGITYNKLSFGSPGRLSKTLGQVLYYQSSMSWDTYLKAGFVEK | |
| | WKPFTPNWMNNTVISRPNQGNCPRYHKCPEICYGGTYNDIA | |
| | PLDLGKDMYVSVILDSDQLAENPEITVFNSTTILYKERVSKDELN | |
| | TRSTTTSCFLFLDEPWCISVLETNRFNGKSIRPEIYSYKIPKYC | |
| 140 | MPQKTVEFINMNSPLERGVSTLSDKKTLNQSKITKQGYFGLGS | gb:NC_025256:9117- |
| | HSERNWKKQKNQNDHYMTVSTMILEILVVLGIMFNLIVLTMV | 11015\|Organism:Bat |
| | YYQNDNINQRMAELTSNITVLNLNLNQLTNKIQREIIPRITLIDT | Paramyxovirus |
| | ATTITIPSAITYILATLTTRISELLPSINQKCEFKTPTLVLNDCRINCT | Eid_hel/GH- |
| | PPLNPSDGVKMSSLATNLVAHGPSPCRNFSSVPTIYYYRIPGLY | M74a/GHA/2009\|Strain |
| | NRTALDERCILNPRLTISSTKFAYVHSEYDKNCTRGFKYYELMTF | Name:BatPV/Eid_hel/GH- |
| | GEILEGPEKEPRMFSRSFYSPTNAVNYHSCTPIVTVNEGYFLCLE | M74a/GHA/2009\|Protein |
| | CTSSDPLYKANLSNSTFHLVILRHNKDEKIVSMPSFNLSTDQEY | Name:glycoprotein\| |
| | VQIIPAEGGGTAESGNLYFPCIGRLLHKRVTHPLCKKSNCSRTD | Gene Symbol:G |
| | DESCLKSYYNQGSPQHQVVNCLIRIRNAQRDNPTWDVITVDL | |
| | TNTYPGSRSRIFGSFSKPMLYQSSVSWHTLLQVAEITDLDKYQL | |
| | DWLDTPYISRPGGSECPFGNYCPTVCWEGTYNDVYSLTPNND | |
| | LFVTVYLKSEQVAENPYFAIFSRDQILKEFPLDAWISSARTTTISC | |
| | FMFNNEIWCIAALEITRENDDIIRPIYYSFWLPTDCRTPYPHTGK | |
| | MTRVPLRSTYNY | |
| 141 | MATNRDNTITSAEVSQEDKVKKYYGVETAEKVADSISGNKVFIL | gb:NC_025352:8716- |
| | MNTLLILTGAIITITLNITNLTAAKSQQNMLKIIQDDVNAKLEMF | 11257\|Organism:Mojiang |
| | VNLDQLVKGEIKPKVSLINTAVSVSIPGQISNLQTKFLQKYVYLE | virus\|Strain |
| | ESITKQCTCNPLSGIFPTSGPTYPPTDKPDDDTTDDDKVDTTIKP | Name:Tongguan1\| |
| | IEYPKPDGCNRTGDHFTMEPGANFYTVPNLGPASSNSDECYT | Protein Name: |
| | NPSFSIGSSIYMFSQEIRKTDCTAGEILSIQIVLGRIVDKGQQGP | attachment |
| | QASPLLVWAVPNPKIINSCAVAAGDEMGWVLCSVTLTAASGE | glycoprotein\|Gene |
| | PIPHMFDGFWLYKLEPDTEVVSYRITGYAYLLDKQYDSVFIGKG | Symbol:G |
| | GGIQKGNDLYFQMYGLSRNRQSFKALCEHGSCLGTGGGGYQ | |
| | VLCDRAVMSFGSEESLITNAYLKVNDLASGKPVIIGQTFPPSDSY | |
| | KGSNGRMYTIGDKYGLYLAPSSWNRYLRFGITPDISVRSTTWL | |
| | KSQDPIMKILSTCTNTDRDMCPEICNTRGYQDIFPLSEDSEYYT | |
| | YIGITPNNGGTKNFVAVRDSDGHIASIDILQNYYSITSATISCFM | |
| | YKDEIWCIAITEGKKQKDNPQRIYAHSYKIRQMCYNMKSATVT | |
| | VGNAKNITIRRY | |
| 142 | FNTVIALLGS IVIIVMNIMI IQNYTRSTDN QAVIKDALQG | NivG protein |
| | IQQQIKGLAD KIGTEIGPKV SLIDTSSTIT IPANIGLLGS | attachment |
| | KISQSTASIN ENVNEKCKFT LPPLKIHECN ISCPNPLPFR | glycoprotein |
| | EYRPQTEGVS NLVGLPNNIC LQKTSNQILK PKLISYTLPV | Without cytoplasmic |
| | VGQSGTCITD PLLAMDEGYF AYSHLERIGS CSRGVSKQRI | tail |
| | IGVGEVLDRG DEVPSLFMTN VWTPPNPNTV YHCSAVYNNE | Uniprot Q9IH62 |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 143 | FNTVIALLGSI IIIVMNIMII QNYTRTTDNQ ALIKESLQSV | Hendra virus G protein |
| | QQQIKALTDK IGTEIGPKVS LIDTSSTITI PANIGLLGSK | Uniprot O89343 |
| | ISQSTSSINE NVNDKCKFTL PPLKIHECNI SCPNPLPFRE | Without cytoplasmic |
| | YRPISQGVSD LVGLPNQICL QKTTSTILKP RLISYTLPIN | tail |
| | TREGVCITDP LLAVDNGFFA YSHLEKIGSC TRGIAKQRII | |
| | GVGEVLDRGD KVPSMFMTNV WTPPNPSTIH HCSSTYHEDF | |
| | YYTLCAVSHV GDPILNSTSW TESLSLIRLA VRPKSDSGDY | |
| | NQKYIAITKV ERGKYDKVMP YGPSGIKQGD TLYFPAVGFL | |
| | PRTEFQYNDS NCPIIHCKYS KAENCRLSMG VNSKSHYILR | |
| | SGLLKYNLSL GGDIILQFIE IADNRLTIGS PSKIYNSLGQ | |
| | PVFYQASYSW DTMIKLGDVD TVDPLRVQWR NNSVISRPGQ | |
| | SQCPRFNVCP EVCWEGTYND AFLIDRLNWV SAGVYLNSNQ | |
| | TAENPVFAVF KDNEILYQVP LAEDDTNAQK TITDCFLLEN | |
| | VIWCISLVEI YDTGDSVIRP KLFAVKIPAQ CSES | |
| 144 | MVVILDKRCY CNLLILILMI SECSVG | Signal sequence |
| 145 | GGGGGS | Peptide linker |
| 146 | (GGGGS)n wherein n is 1 to 10 | Peptide linker |
| 147 | GGGGS | Peptide linker |
| 148 | PAENKKVR FENTTSDKGK IPSKVIKSYY GTMDIKKINE | NiVG protein |
| | GLLDSKILSA FNTVIALLGS IVIIVMNIMI IQNYTRSTDN | attachment |
| | QAVIKDALQG IQQQIKGLAD KIGTEIGPKV SLIDTSSTIT | glycoprotein (602 aa) |
| | IPANIGLLGS KISQSTASIN ENVNEKCKFT LPPLKIHECN | Without N-terminal |
| | ISCPNPLPFR EYRPQTEGVS NLVGLPNNIC LQKTSNQILK | methionine |
| | PKLISYTLPV VGQSGTCITD PLLAMDEGYF AYSHLERIGS | |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRFNTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QC | |
| 149 | KVR FENTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS | glycoprotein |
| | KISQSTASIN ENVNEKCKFT LPPLKIHECN ISCPNPLPFR | Truncated Δ5 Without |
| | EYRPQTEGVS NLVGLPNNIC LQKTSNQILK PKLISYTLPV | N-terminal |
| | VGQSGTCITD PLLAMDEGYF AYSHLERIGS CSRGVSKQRI | methionine |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 150 | NTTSDKGK IPSKVIKSYY GTMDIKKINE GLLDSKILSA | NiVG protein |
| | FNTVIALLGS IVIIVMNIMI IQNYTRSTDN QAVIKDALQG | attachment |
| | IQQQIKGLAD KIGTEIGPKV SLIDTSSTIT IPANIGLLGS | glycoprotein |
| | KISQSTASIN ENVNEKCKFT LPPLKIHECN ISCPNPLPFR | Truncated Δ10 |
| | EYRPQTEGVS NLVGLPNNIC LQKTSNQILK PKLISYTLPV | Without N-terminal |
| | VGQSGTCITD PLLAMDEGYF AYSHLERIGS CSRGVSKQRI | methionine |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 151 | KGK IPSKVIKSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ15 |
| | NLVGLPNNIC LQKTSNQILK PKLISYTLPV VGQSGTCITD | Without N-terminal |
| | PLLAMDEGYF AYSHLERIGS CSRGVSKQRI IGVGEVLDRG | methionine |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 152 | SKVIKSYY GTMDIKKINE GLLDSKILSA FNTVIALLGS | NiVG protein |
| | IVIIVMNIMI IQNYTRSTDN QAVIKDALQG IQQQIKGLAD | attachment |
| | KIGTEIGPKV SLIDTSSTIT IPANIGLLGS KISQSTASIN | glycoprotein |
| | ENVNEKCKFT LPPLKIHECN ISCPNPLPFR EYRPQTEGVS | Truncated Δ20 |
| | NLVGLPNNIC LQKTSNQILK PKLISYTLPV VGQSGTCITD | Without N-terminal |
| | PLLAMDEGYF AYSHLERIGS CSRGVSKQRI IGVGEVLDRG | methionine |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 153 | SYY GTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ25 |
| | LQKTSNQILK PKLISYTLPV VGQSGTCITD PLLAMDEGYF | Without N-terminal |
| | AYSHLERIGS CSRGVSKQRI IGVGEVLDRG DEVPSLFMTN | methionine |
| | VWTPPNPNTV YHCSAVYNNE FYYVLCAVST VGDPILNSTY | |
| | WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 154 | TMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN ISCPNPLPFR EYRPQTEGVS NLVGLPNNIC | Truncated Δ30 |
| | LQKTSNQILK PKLISYTLPV VGQSGTCITD PLLAMDEGYF | Without N-terminal |
| | AYSHLERIGS CSRGVSKQRI IGVGEVLDRG DEVPSLFMTN | methionine |
| | VWTPPNPNTV YHCSAVYNNE FYYVLCAVST VGDPILNSTY | |
| | WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 155 | KKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT | attachment |
| | IPANIGLLGS KISQSTASIN ENVNEKCKFT LPPLKIHECN | glycoprotein |
| | ISCPNPLPFR EYRPQTEGVS NLVGLPNNIC LQKTSNQILK | Truncated and |
| | PKLISYTLPV VGQSGTCITD PLLAMDEGYF AYSHLERIGS | mutated |
| | CSRGVSKQRI IGVGEVLDRG DEVPSLFMTN VWTPPNPNTV | (E501 A, W504A, |
| | YHCSAVYNNE FYYVLCAVST VGDPILNSTY WSGSLMMTRL | Q530A, E533A) NiV G |
| | AVKPKSNGGG YNQHQLALRS IEKGRYDKVM PYGPSGIKQG | protein (Gc Δ 34) |
| | DTLYFPAVGF LVRTEFKYND SNCPITKCQY SKPENCRLSM | Without N-terminal |
| | GIRPNSHYIL RSGLLKYNLS DGENPKVVFI EISDQRLSIG | methionine |
| | SPSKIYDSLG QPVFYQASFS WDTMIKFGDV LTVNPLVVNW | |
| | RNNTVISRPG QSQCPRFNTC PAICAEGVYN DAFLIDRINW | |
| | ISAGVFLDSN ATAANPVFTV FKDNEILYRA QLASEDTNAQ | |
| | KTITNCFLLK NKIWCISLVE IYDTGDNVIR PKLFAVKIPE QCT | |
| 156 | MADSKLVSL NNNLSGKIKD QGKVIKNYYG TMDIKKINDG | Hendra virus G protein |
| | LLDSKILGAF NTVIALLGSI IIIVMNIMII QNYTRTTDNQ | Uniprot O89343 |
| | ALIKESLQSV QQQIKALTDK IGTEIGPKVS LIDTSSTITI | Without N-terminal |
| | PANIGLLGSK ISQSTSSINE NVNDKCKFTL PPLKIHECNI | methionine |
| | SCPNPLPFRE YRPISQGVSD LVGLPNQICL QKTTSTILKP | |
| | RLISYTLPIN TREGVCITDP LLAVQNGFFA YSHLEKIGSC | |
| | TRGIAKQRII GVGEVLDRGD KVPSMFMTNV WTPPNPSTIH | |
| | HCSSTYHEDF YYTLCAVSHV GDPILNSTSW TESLSLIRLA | |
| | VRPKSDSGDY NQKYIAITKV ERGKYDKVMP YGPSGIKQGD | |
| | TLYFPAVGFL PRTEFQYNDS NCPIIHCKYS KAENCRLSMG | |
| | VNSKSHYILR SGLLKYNLSL GGDIILQFIE IADNRLTIGS | |
| | PSKIYNSLGQ PVFYQASYSW DTMIKLGDVD TVDPLRVQWR | |
| | NNSVISRPGQ SQCPRFNVCP EVCWEGTYND AFLIDRLNWV | |
| | SAGVYLNSNQ TAENPVFAVF KDNEILYQVP LAEDDTNAQK | |
| | TITDCFLLEN VIWCISLVEI YDTGDSVIRP KLFAVKIPAQ CSES | |
| 157 | KKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT | attachment |
| | IPANIGLLGS KISQSTASIN ENVNEKCKFT LPPLKIHECN | glycoprotein |
| | ISCPNPLPFR EYRPQTEGVS NLVGLPNNIC LQKTSNQILK | Truncated (Gc Δ 34) |
| | PKLISYTLPV VGQSGTCITD PLLAMDEGYF AYSHLERIGS | Without N-terminal |
| | CSRGVSKQRI IGVGEVLDRG DEVPSLFMTN VWTPPNPNTV | methionine |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRFNTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QCT | |
| 158 | LSQLQKNYLDNSNQQGDKMNNPDKKLSVNFNPLELDKGQKD | gb:JQ001776:8170- |
| | LNKSYYVKNKNYNVSNLLNESLHDIKFCIYCIFSLLIIITIINIITISIVI | 10275\|Organism:Cedar |
| | TRLKVHEENNGMESPNLQSIQDSLSSLTNMINTEITPRIGILVTA | virus\|Strain |
| | TSVTLSSSINYVGTKTNQLVNELKDYITKSCGFKVPELKLHECNIS | Name:CG1a\|Protein |
| | CADPKISKSAMYSTNAYAELAGPPKIFCKSVSKDPDFRLKQIDY | Name:attachment |
| | VIPVQQDRSICMNNPLLDISDGFFTYIHYEGINSCKKSDSFKVLL | glycoprotein\|Gene |
| | SHGEIVDRGDYRPSLYLLSSHYHPYSMQVINCVPVTCNQSSFVF | Symbol:G Without N- |
| | CHISNNTKTLDNSDYSSDEYYITYFNGIDRPKTKKIPINNMTAD | terminal methionine |
| | NRYIHFTFSGGGGVCLGEEFIIPVTTVINTDVFTHDYCESFNCSV | |
| | QTGKSLKEICSESLRSPTNSSRYNLNGIMIISQNNMTDFKIQLN | |
| | GITYNKLSFGSPGRLSKTLGQVLYYQSSMSWDTYLKAGFVEKW | |
| | KPFTPNWMNNTVISRPNQGNCPRYHKCPEICYGGTYNDIAPL | |
| | DLGKDMYVSVILDSDQLAENPEITVFNSTTILYKERVSKDELNTR | |
| | STTTSCFLFLDEPWCISVLETNRFNGKSIRPEIYSYKIPKYC | |
| 159 | PQKTVEFINMNSPLERGVSTLSDKKTLNQSKITKQGYFGLGSHS | gb:NC_025256:9117- |
| | ERNWKKQKNQNDHYMTVSTMILEILVVLGIMFNLIVLTMVYY | 11015\|Organism:Bat |
| | QNDNINQRMAELTSNITVLNLNLNQLTNKIQREIIPRITLIDTAT | Paramyxovirus |
| | TITIPSAITYILATLTTRISELLPSINQKCEFKTPTLVLNDCRINCTPP | Eid_hel/GH- |
| | LNPSDGVKMSSLATNLVAHGPSPCRNFSSVPTIYYYRIPGLYNR | M74a/GHA/2009\|Strain |
| | TALDERCILNPRLTISSTKFAYVHSEYDKNCTRGFKYYELMTFGE | Name:BatPV/Eid_hel/GH- |
| | ILEGPEKEPRMFSRSFYSPTNAVNYHSCTPIVTVNEGYFLCLECT | M74a/GHA/2009\|Protein |
| | SSDPLYKANLSNSTFHLVILRHNKDEKIVSMPSFNLSTDQEYVQI | Name:glycoprotein\| |
| | IPAEGGGTAESGNLYFPCIGRILHKRVTHPLCKKSNCSRTDDES | Gene Symbol:G Without |
| | CLKSYYNQGSPQHQVVNCLIRIRNAQRDNPTWDVITVDLTNT | N-terminal |
| | YPGSRSRIFGSFSKPMLYQSSVSWHTLLQVAEITDLDKYQLDW | methionine |
| | LDTPYISRPGGSECPFGNYCPTVCWEGTYNDVYSLTPNNDLFV | |
| | TVYLKSEQVAENPYFAIFSRDQILKEFPLDAWISSARTTTISCFM | |
| | FNNEIWCIAALEITRLNDDIIRPIYYSFWLPTDCRTPYPHTGKMT | |
| | RVPLRSTYNY | |
| 160 | ATNRDNTITSAEVSQEDKVKKYYGVETAEKVADSISGNKVFILM | gb:NC_025352:8716- |
| | NTLLILTGAIITITLNITNLTAAKSQQNMLKIIQDDVNAKLEMFV | 11257\|Organism:Mojiang |
| | NLDQLVKGEIKPKVSLINTAVSVSIPGQISNLQTKFLQKYVYLEES | virus\|Strain |
| | ITKQCTCNPLSGIFPTSGPTYPPTDKPDDDTTDDDKVDTTIKPIE | Name:Tongguan1\|Protein |
| | YPKPDGCNRTGDHFTMEPGANFYTVPNLGPASSNSDECYTNP | Name:attachment |
| | SFSIGSSIYMFSQEIRKTDCTAGEILSIQIVLGRIVDKGQQGPQAS | glycoprotein\|Gene |
| | PLLVWAVPNPKIINSCAVAAGDEMGWVLCSVTLTAASGEPIP | Symbol:G Without N- |
| | HMFDGFWLYKLEPDTEVVSYRITGYAYLLDKQYDSVFIGKGGG | terminal methionine |
| | IQKGNDLYFQMYGLSRNRQSFKALCEHGSCLGTGGGGYQVLC | |
| | DRAVMSFGSEESLITNAYLKVNDLASGKPVIIGQTFPPSDSYKG | |
| | SNGRMYTIGDKYGLYLAPSSWNRYLRFGITPDISVRSTTWLKS | |
| | QDPIMKILSTCTNTDRDMCPEICNTRGYQDIFPLSEDSEYYTYIG | |
| | ITPNNGGTKNFVAVRDSDGHIASIDILQNYYSITSATISCFMYKD | |
| | EIWCIAITEGKKQKDNPQRIYAHSYKIRQMCYNMKSATVTVG | |
| | NAKNITIRRY | |
| 161 | DFDKLNKIGVVQGRVLNYKIKGDPMTKDLVLKFIPNIVNITECV | gb:JQ001776:6129- |
| | REPLSRYNETVRRLLLPIHNMLGLYLNNTNAKMTGLMIAGVIM | 8166\|Organism:Cedar |
| | GGIAIGIATAAQITAGFALYEAKKNTENIQKLTDSIMKTQDSIDK | virus\|Strain |
| | LTDSVGTSILILNKLQTYINNQLVPNLELLSCRQNKIEFDLMLTKY | Name:CG1a\|Protein |
| | LVDLMTVIGPNINNPVNKDMTIQSLSLLFDGNYDIMMSELGYT | Name:fusion |
| | PQDFLDLIESKSITGQIIYVDMENLYVVIRTYLPTLIEVPDAQIYEF | glycoprotein\|Gene |
| | NKITMSSNGGEYLSTIPNFILIRGNYMSNIDVATCYMTKASVIC | Symbol:F (without |
| | NQDYSLPMSQNLRSCYQGETEYCPVEAVIASHSPRFALTNGVI | signal sequence) |
| | FANCINTICRCQDNGKTITQNINQFVSMIDNSTCNDVMVDKF | |
| | TIKVGKYMGRKDINNINIQIGPQIIIDKVDLSNEINKMNQSLKDS | |
| | IFYLREAKRILDSVNISLISPSVQLFLIIISVLSFIILLIIIVYLYCKSKHS | |
| | YKYNKFIDDPDYYNDYKRERINGKASKSNNIYYVGD | |
| 162 | SRALLRETDNYSNGLIVENLVRNCHHPSKNNLNYTKTQKRDSTI | gb:NC_025256:6865- |
| | PYRVEERKGHYPKIKHLIDKSYKHIKRGKRRNGHNGNIITHILLLIL | 8853\|Organism:Bat |
| | ILKTQMSEGAIHYETLSKIGLIKGITREYKVKGTPSSKDIVIKLIPN | Paramyxovirus |
| | VTGLNKCTNISMENYKEQLDKILIPINNIIELYANSTKSAPGNAR | Eid_hel/GH- |
| | FAGVIIAGVALGVAAAAQITAGIALHEARQNAERINLLKDSISAT | M74a/GHA/2009\|Strain |
| | NNAVAELQEATGGIVNVITGMQDYINTNLVPQIDKLQCSQIKT | Name:BatPV/Eid_hel/GH- |
| | ALDISLSQYYSEILTVFGPNLQNPVTTSMSIQAISQSFGGNIDLLL | M74a/GHA/2009\|Protein |
| | NLLGYTANDLLDLLESKSITGQITYINLEHYFMVIRVYYPIMTTIS | Name:fusion |
| | NAYVQELIKISFNVDGSEWVSLVPSYILIRNSYLSNIDISECLITKN | protein\|Gene |
| | SVICRHDFAMPMSYTLKECLTGDTEKCPREAVVTSYVPRFAISG | Symbol:F (without |
| | GVIYANCLSTTCQCYQTGKVIAQDGSQTLMMIDNQTCSIVRIE | signal sequence) |
| | EILISTGKYLGSQEYNTMHVSVGNPVFTDKLDITSQISNINQSIE | |
| | QSKFYLDKSKAILDKINLNLIGSVPISILFIIAILSLILSIITFVIVMIIVR | |
| | RYNKYTPLINSDPSSRRSTIQDVYIIPNPGEHSIRSAARSIDRDRD | |
| 163 | ILHY EKLSKIGLVK GITRKYKIKS NPLTKDIVIK MIPNVSNVSK | Hendra virus F protein |
| | CTGTVMENYK SRLTGILSPI KGAIELYNNN THDLVGDVKL | Uniprot O89342 |
| | AGVVMAGIAI GIATAAQITA GVALYEAMKN ADNINKLKSS | (without signal |
| | IESTNEAVVK LQETAEKTVY VLTALQDYIN TNLVPTIDQI | sequence) |
| | SCKQTELALD LALSKYLSDL LFVFGPNLQD PVSNSMTIQA | |
| | ISQAFGGNYE TLLRTLGYAT EDFDDLLESD SIAGQIVYVD | |
| | LSSYYIIVRV YFPILTEIQQ AYVQELLPVS FNNDNSEWIS | |
| | IVPNFVLIRN TLISNIEVKY CLITKKSVIC NQDYATPMTA | |
| | SVRECLTGST DKCPRELVVS SHVPRFALSG GVLFANCISV | |
| | TCQCQTTGRA ISQSGEQTLL MIDNTTCTTV VLGNIIISLG | |
| | KYLGSINYNS ESIAVGPPVY TDKVDISSQI SSMNQSLQQS | |
| | KDYIKEAQKI LDTVNPSLIS MLSMIILYVL SIAALCIGLI | |
| | TFISFVIVEK KRGNYSRLDD RQVRPVSNGD LYYIGT | |
| 164 | IHYDSLSKVGVIKGLTYNYKIKGSPSTKLMVVKLIPNIDSVKNCT | gb:NC_025352:5950- |
| | QKQYDEYKNLVRKALEPVKMAIDTMLNNVKSGNNKYRFAGAI | 8712\|Organism:Mojiang |
| | MAGVALGVATAATVTAGIALHRSNENAQAIANMKSAIQNTN | virus\|Strain |
| | EAVKQLQLANKQTLAVIDTIRGEINNNIIPVINQLSCDTIGLSVGI | Name:Tongguan1\| |
| | RLTQYYSEIITAFGPALQNPVNTRITIQAISSVFNGNFDELLKIM | Protein Name:fusion |
| | GYTSGDLYEILHSELIRGNIIDVDVDAGYIALEIEFPNLTLVPNAV | protein\|Gene |
| | VQELMPISYNIDGDEWVTLVPRFVLTRTTLLSNIDTSRCTITDSS | Symbol:F (without |
| | VICDNDYALPMSHELIGCLQGDTSKCAREKVVSSYVPKFALSDG | signal sequence) |
| | LVYANCLNTICRCMDTDTPISQSLGATVSLLDNKRCSVYQVGD | |
| | VLISVGSYLGDGEYNADNVELGPPIVIDKIDIGNQLAGINQTLQ | |
| | EAEDYIEKSEEFLKGVNPSIITLGSMVVLYIFMILIAIVSVIALVLSI | |
| | KLTVKGNVVRQQFTYTQHVPSMENINYVSH | |
| 165 | (GGGS)n wherein n is 1 to 10 | Peptide linker |
| 32 | GGGGSGGGGSGGGGS | Peptide linker |
| 166 | TTAASGSSGGSSSGA | Peptide linker |
| 33 | GSTSGSGKPGSGEGSTKG | Peptide linker |
| | GS | Peptide linker |
| | GGS | Peptide linker |
| | GGGS | Peptide linker |

TABLE 27
Exemplary sequences of CD47

| SEQ ID | | |
|---|---|---|
| NO: | Sequence | Description |
| 167 | MWPLVAALLLGSACCGSAQLLFNKTKSVEFTFCNDTVVIPCFVTNM | Amino acid |
| | EAQNTTEVYVKWKFKGRDIYTFDGALNKSTVPTDESSAKIEVSQLLK | sequence |
| | GDASLKMDKSDAVSHTGNYTCEVTELTREGETIIELKYRVVSWFSPN | encoded by |
| | ENILIVIFPIFAILLFWGQFGIKTLKYRSGGMDEKTIALLVAGLVITVIVI | CDS of |
| | VGAILFVPGEYSLKNATGLGLIVTSTGILILLHYYVFSTAIGLTSFVIAILV | NM_001777.4 |
| | IQVIAYILAVVGLSLCIAACIPMHGPLLISGLSILALAQLLGLVYMKFVA | |
| | SNQKTIQPPRKAVEEPLNAFKESKGMMNDE | |
| 168 | MWPLVAALLLGSACCGSAQLLFNKTKSVEFTFCNDTVVIPCFVTNM | Amino acid |
| | EAQNTTEVYVKWKFKGRDIYTFDGALNKSTVPTDFSSÅKIEVSQLLK | sequence |
| | GDASLKMDKSDAVSHTGNYTCEVTELTREGETIIELKYRVVSWFSPN | encoded by |
| | ENILIVIFPIFAILLFWGQFGIKTLKYRSGGMDEKTIALLVAGLVITVIVI | CDS of |
| | VGAILFVPGEYSLKNATGLGLIVTSTGILILLHYYVFSTAIGLTSFVIAILV | NM_198793.2 |
| | IQVIAYILAVVGLSLCIAACIPMHGPLLISGLSILALAQLLGLVYMKFVA | |
| | SNQKTIOPPRNN | |
| 169 | atgtggcccctggtagcggcgctgttgctgggctcggcgtgctgcggatcagctcagc | Nucleotide |
| | tactatttaataaaacaaaatctgtagaattcacgttttgtaatgacactgtcgtcattc | sequence of |
| | catgctttgttactaatatggaggcacaaaacactactgaagtatacgtaaagtgga | NM_001777.4 |
| | aatttaaaggaagagatatttacacctttgatggagctctaaacaagtccactgtccc | CDS (nts 124- |
| | cactgactttagtagtgcaaaaattgaagtctcacaattactaaaaggagatgcctct | 1095) |
| | ttgaagatggataagagtgatgctgtctcacacacaggaaactacacttgtgaagta | |
| | acagaattaaccagagaaggtgaaacgatcatcgagctaaaatatcgtgttgtttca | |
| | tggttttctccaaatgaaaatattcttattgttattttcccaatttttgctatactcctgtt | |
| | ctggggacagtttggtattaaaacacttaaatatagatccggtggtatggatgagaa | |
| | aacaattgctttacttgttgctggactagtgatcactgtcattgtcattgttggagccat | |
| | tcttttcgtcccaggtgaatattcattaaagaatgctactggccttggtttaattgtgac | |
| | ttctacagggatattaatattacttcactactatgtgtttagtacagcgattggattaac | |
| | ctccttcgtcattgccatattggttattcaggtgatagcctatatcctcgctgtggttgg | |
| | actgagtctctgtattgcggcgtgtataccaatgcatggccctcttctgatttcaggttt | |
| | gagtatcttagctctagcacaattacttggactagtttatatgaaatttgtggcttccaa | |
| | tcagaagactatacaacctcctaggaaagctgtagaggaaccccttaatgcattcaa | |
| | agaatcaaaaggaatgatgaatgatgaataa | |
| 170 | atgtggcccctggtagcggcgctgttgctgggctcggcgtgctgcggatcagctcagc | Nucleotide |
| | tactatttaataaaacaaaatctgtagaattcacgttttgtaatgacactgtcgtcattc | sequence of |
| | catgctttgttactaatatggaggcacaaaacactactgaagtatacgtaaagtgga | NM 198793.2 |
| | aatttaaaggaagagatatttacacctttgatggagctctaaacaagtccactgtccc | CDS (nts 181- |
| | cactgactttagtagtgcaaaaattgaagtctcacaattactaaaaggagatgcctct | 1098) |
| | ttgaagatggataagagtgatgctgtctcacacacaggaaactacacttgtgaagta | |
| | acagaattaaccagagaaggtgaaacgatcatcgagctaaaatatcgtgttgtttca | |
| | tggttttctccaaatgaaaatattcttattgttattttcccaatttttgctatactcctg | |
| | ttctggggacagtttggtattaaaacacttaaatatagatccggtggtatggatgagaa | |
| | aacaattgctttacttgttgctggactagtgatcactgtcattgtcattgttggagccat | |
| | tcttttcgtcccaggtgaatattcattaaagaatgctactggccttggtttaattgtgac | |
| | ttctacagggatattaatattacttcactactatgtgtttagtacagcgattggattaac | |
| | ctccttcgtcattgccatattggttattcaggtgatagcctatatcctcgctgtggttgg | |
| | actgagtctctgtattgcggcgtgtataccaatgcatggccctcttctgatttcaggttt | |
| | gagtatcttagctctagcacaattacttggactagtttatatgaaatttgtggcttccaa | |
| | tcagaagactatacaacctcctaggaataactga | |
| 171 | atgtggcccctggtcgccgccctgttgctgggctcggcatgctgcggatcagctcagct | |
| | actgtttaataaaacaaaatctgtagaattcacgttttgtaacgacactgtcgtgatcc | Codon- |
| | catgctttgttactaatatggaggcacaaaacaccactgaagtgtacgtgaagtgga | optimized |
| | aattcaaaggcagagacatttacacctttgacggcgccctcaacaagtccaccgtgc | nucleotide |
| | ccactgactttagtagcgcaaaaattgaggtcagccaattactaaaaggagatgcct | sequence |
| | ctttgaagatggacaagagcgatgctgtcagccacacagggaactacacttgtgaag | encoding |
| | taacagagttaacccgcgaaggtgaaacgatcatcgagctgaagtatcgagtggtgt | CD47 |
| | cctggttttctccgaacgagaatatccttatcgtaattttcccaattttcgctatcctcct | |
| | gttctggggccagtttggtatcaagacactcaaatatcggtccggtgggatggatgag | |
| | aagacaattgccctgcttgttgctggactcgtgatcaccgtcatcgtgattgttggggc | |
| | catccttttcgtcccaggggagtacagcctgaagaatgctacgggcctgggattaatt | |
| | gtgacctctacagggatactcatcctgcttcactactatgtgttcagtaccgcgattgg | |
| | actgacctccttcgtcattgccatattggtgattcaggtgatagcctacatcctcgccgt | |
| | ggttggcctgagtctctgtatcgcggcgtgcatacccatgcatggccctcttctgatttc | |
| | agggttgagtatcctcgcactagcacagttgctgggactggtttatatgaaatttgtgg | |
| | cctccaaccagaagactatacagcctcctaggaaggctgtagaggagcccctgaatg | |
| | cattcaaggaatcaaaaggcatgatgaatgatgaa | |

TABLE 28
Sequences of 2A peptides

| SEQ ID NO: | Amino Acid Sequence | 2A Peptide |
|---|---|---|
| 172 | (GSG) EGRGSLLTCGDVEENPGP | T2A |
| 173 | (GSG) ATNFSLLKQAGDVEENPGP | P2A |
| 174 | (GSG) QCTNYALLKLAGDVESNPGP | E2A |
| 175 | (GSG) VKQTLNFDLLKLAGDVESNPGP | F2A |

TABLE 29
Sequences of furin sites

| SEQ ID | | Furin |
|---|---|---|
| NO: | Amino Acid Sequence | site |
| 176 | RRRR (GSG) | FC1 |
| 177 | RKRR (GSG) | FC2 |
| 178 | RKRR (GSG) TPDPW (GSG) | FC3 |

TABLE 30
Exemplary Cas nuclease variants and
their PAM sequences

| | | PAM |
|---|---|---|
| CRISPR | | Sequence |
| Nuclease | Source Organism | (5′-3′) |
| SpCas9 | Streptococcus pyogenes | ngg or nag |
| SaCas9 | Staphylococcus aureus | ngrrt or |
| | | ngrn |
| NmeCas9 | Neisseria meningitidis | nnnngatt |
| CjCas9 | Campylobacter jejuni | nnnnryac |
| StCas9 | Streptococcus | nnagaaw |
| | thermophilus | |
| TdCas9 | Treponema denticola | naaaac |
| LbCas12a | Lachnospiraceae | tttv |
| (Cpf1) | bacterium | |
| AsCas12a | Acidaminococcus sp. | tttv |
| (Cpf1) | | |
| AacCas12b | Alicyclobacillus | ttn |
| | acidiphilus | |
| BhCas12bv4 | Bacillus hisashil | attn, tttn, |
| | | or gttn |

r = a org; y = cort; w = a ort; v = a or c org; n = any base

TABLE 31
Exemplary gRNA structure and sequence for CRISPR/Cas

| SEQ ID | | |
|---|---|---|
| NO | Sequence (5′-3′) | Description |
| | nnnnnnnnnnnnnnnnnnnn | Exemplary |
| | | spCas9 1 |
| | | Complementary |
| | | region (spacer) |
| 180 | guuuuagagcua | Exemplary |
| | | spCas9 1 crRNA |
| | | repeat region |
| | gaaa | Exemplary |
| | | spCas9 t |
| | | tetraloop |
| 182 | Uagcaaguuaaaauaaggcuaguccguuaucaacuugaaaaaguggca | Exemplary |
| | | spCas9 1 |
| | | tracrRNA |
| | | Exemplary |
| | | spCas9 2 |
| | | Complementary |
| | | region (spacer) |
| 184 | guuusagagcuaugcug | Exemplary |
| | | spCas9 2 crRNA |
| | | repeat region |
| | gaaa | Exemplary |
| | | spCas9 2 |
| | | tetraloop |
| 186 | cagcauagcaaguusaaauaaggcuaguccguuaucaacuugaaaaagu | Exemplary |
| | ggcaccgagucggugcuuuuuu | spCas9 2 |
| | | tracrRNA |
| | | Exemplary |
| | | saCas9 |
| | | Complementary |
| | | region |
| 188 | guuuuaguacucug | Exemplary |
| | | saCas9 crRNA |
| | | repeat region |
| | gaaa | Exemplary |
| | | saCas9 tetraloop |
| 190 | cagaaucuacuaaaacaaggcaaaaugccguguuuaucucgucaacuug | Exemplary |
| | uuggcgagauuuuuu | saCas9 tracrRNA |
| 191 | gucgucuauaggacggcgaggacaacgggaagugccaaugugcucuuuc | Exemplary |
| | caagagcaaacaccccguuggcuucaagaugaccgcucg | AkCas12b |
| | | tracrRNA |
| | aaaa | Exemplary |
| | | AkCas12b |
| | | tetraloop |
| 193 | cgagcggucugagaaguggcacu | Exemplary |
| | | AkCas12b crDNA |
| | | repeat region |
| | | Exemplary |
| | | AkCas12b |
| | | Complementary |
| | | region (spacer) |

s = corg; n = any base

TABLE 32
CD19 CARs Used in Experimental Groups

| Experimental | | VH SEQ | VL SEQ |
|---|---|---|---|
| Group | Construct | ID NO: | ID NO: |
| Mock | GFP | — | — |
| Control | FMC63 | — | — |
| scFv | CAR 369 (VH-VL) | 19 | 22 |
| | CAR 399 (VL-VH) | 20 | 23 |
| | CAR 400 (VL-VH) | 21 | 24 |
| | CAR 400 (VH-VL) | 21 | 24 |

TABLE 33
Experimental Groups for in vivo tumor challenge in Example 3.

| | Mouse | Tumor | Tumor | CAR-T | CAR-T cell | |
|---|---|---|---|---|---|---|
| Group | Strain | Challenge | cells/Animal | cells | dose | Mice/Group |
| 1 | NSG | Nalm6:Wasabi- | 5.00E+05 | CAR 369 | 4.00E+06 | 5 |
| | | ffLuc | | (VH-VL) | | |
| 2 | NSG | Nalm6:Wasabi- | 5.00E+05 | CAR 400 | 4.00E+06 | 5 |
| | | ffLuc | | (VH-VL) | | |
| 3 | NSG | Nalm6:Wasabi- | 5.00E+05 | CAR 399 | 4.00E+06 | 5 |
| | | ffLuc | | (VL-VH) | | |
| 4 | NSG | Nalm6:Wasabi- | 5.00E+05 | CAR 400 | 4.00E+06 | 10 |
| | | ffLuc | | (VL-VH) | | |
| | | | | CAR-T | | |
| 5 | NSG | Nalm6:Wasabi- | 5.00E+05 | FMC63 | 4.00E+06 | 10 |
| | | ffLuc | | | | |
| 6 | NSG | Nalm6:Wasabi- | 5.00E+05 | Mock | 4.00E+06 | 5 |
| | | ffLuc | | | | |
| 7 | NSG | — | — | Mock | 4.00E+06 | 5 |
| 8 | NSG | Nalm6:Wasabi- | 5.00E+05 | — | — | 5 |
| | | ffLuc | | | | |
| 9 | NSG | nn | — | — | — | 3 |

TABLE 34
Experimental Groups in Examples 4 and 5.

| | Mice/ | | Tumor | PBMC/ | | |
|---|---|---|---|---|---|---|
| Group | Group | Target Cell | dose | mouse | Fusosome | IU/Mouse |
| 1 | 5 | Nalm6: | 2.5e+5 | 1.0e+7 | CD8-CAR 400 (VL- | 8.0e+6 |
| | | Wasabi- | cells/mouse | | VH) | |
| | | ffLuc | | | | |
| 2 | 5 | Nalm6: | 2.5e+5 | 1.0e+7 | CD8-CAR 400 (VL- | 5.0e+6 |
| | | Wasabi- | cells/mouse | | VH | |
| | | ffLuc | | | | |
| 3 | 5 | Nalm6: | 2.5e+5 | 1.0e+7 | CD8-CAR 400 (VL- | 2.5e+6 |
| | | Wasabi- | cells/mouse | | VH) | |
| | | ffLuc | | | | |
| 4 | 5 | Nalm6: | 2.5e+5 | 1.0e+7 | CD8-CAR 400 (VL- | 1.0e+6 |
| | | Wasabi- | cells/mouse | | VH | |
| | | ffLuc | | | | |
| 5 | 5 | Nalm6: | 2.5e+5 | 1.0e+7 | CD8-CAR 400 (VL- | 8.0e+6 |
| | | Wasabi- | cells/mouse | | VH | |
| | | ffLuc | | | | |
| 6 | 5 | Nalm6: | 2.5e+5 | 1.0e+7 | CD8-CAR 400 (VL- | 5.0e+6 |
| | | Wasabi- | cells/mouse | | VH) | |
| | | ffLuc | | | | |
| 7 | 5 | Nalm6: | 2.5e+5 | 1.0e+7 | CD8-CAR 400 (VL- | 2.5e+6 |
| | | Wasabi- | cells/mouse | | VH) | |
| | | ffLuc | | | | |
| 8 | 5 | Nalm6: | 2.5e+5 | 1.0e+7 | CD8-CAR 400 (VL- | 1.0e+6 |
| | | Wasabi- | cells/mouse | | VH | |
| | | ffLuc | | | | |
| 9 | 5 | Nalm6: | 2.5e+5 | 1.0e+7 | CD8-CAR 400 (VL- | 8.0e+6 |
| | | Wasabi- | cells/mouse | | VH | |
| | | ffLuc | | | | |
| 10 | 5 | Nalm6: | 2.5e+5 | 1.0e+7 | CD8-CAR 400 (VL- | 5.0e+6 |
| | | Wasabi- | cells/mouse | | VH | |
| | | ffLuc | | | | |
| 11 | 5 | Nalm6: | 2.5e+5 | 1.0e+7 | CD8-CAR 400 (VL- | 2.5e+6 |
| | | Wasabi- | cells/mouse | | VH | |
| | | ffLuc | | | | |
| 12 | 5 | Nalm6: | 2.5e+5 | 1.0e+7 | CD8-CAR 400 (VL- | 1.0e+6 |
| | | Wasabi- | cells/mouse | | VH | |
| | | ffLuc | | | | |
| 13 | 5 | Nalm6: | 2.5e+5 | 1.0e+7 | CD8-CAR 400 (VL- | 8.0e+6 |
| | | Wasabi- | cells/mouse | | VH) | |
| | | ffLuc | | | | |
| 14 | 5 | Nalm6: | 2.5e+5 | 1.0e+7 | CD8-CAR 400 (VL- | 5.0e+6 |
| | | Wasabi- | cells/mouse | | VH) | |
| | | ffLuc | | | | |
| 15 | 5 | Nalm6: | 2.5e+5 | 1.0e+7 | CD8-CAR 400 (VL- | 2.5e+6 |
| | | Wasabi- | cells/mouse | | VH) | |
| | | ffLuc | | | | |
| 16 | 5 | Nalm6: | 2.5e+5 | 1.0e+7 | CD8-CAR 400 (VL- | 1.0e+6 |
| | | Wasabi- | cells/mouse | | VH) | |
| | | ffLuc | | | | |
| 17 | 5 | Nalm6: | 2.5e+5 | 1.0e+7 | CD8-CAR 400 (VL- | 8.0e+6 |
| | | Wasabi- | cells/mouse | | VH) | |
| | | ffLuc | | | | |
| 18 | 5 | Nalm6: | 2.5e+5 | 1.0e+7 | CD8-CAR 400 (VL- | 5.0e+6 |
| | | Wasabi- | cells/mouse | | VH) | |
| | | ffLuc | | | | |
| 19 | 5 | Nalm6: | 2.5e+5 | 1.0e+7 | CD8-CAR 400 (VL- | 2.5e+6 |
| | | Wasabi- | cells/mouse | | VH) | |
| | | ffLuc | | | | |
| 20 | 5 | Nalm6: | 2.5e+5 | 1.0e+7 | CD8-CAR 400 (VL- | 1.0e+6 |
| | | Wasabi- | cells/mouse | | VH) | |
| | | ffLuc | | | | |
| 21 | 5 | Nalm6: | 2.5e+5 | 1.0e+7 | CD8-FMC63 | 8.0e+6 |
| | | Wasabi- | cells/mouse | | | |
| | | ffLuc | | | | |
| 22 | 5 | Nalm6: | 2.5e+5 | 1.0e+7 | CD8-FMC63 | 5.0e+6 |
| | | Wasabi- | cells/mouse | | | |
| | | ffLuc | | | | |
| 23 | 5 | Nalm6: | 2.5e+5 | 1.0e+7 | CD8-FMC63 | 2.5e+6 |
| | | Wasabi- | cells/mouse | | | |
| | | ffLuc | | | | |
| 24 | 5 | Nalm6: | 2.5e+5 | 1.0e+7 | CD8-FMC63 | 1.0e+6 |
| | | Wasabi- | cells/mouse | | | |
| | | ffLuc | | | | |
| 25 | 5 | Nalm6: | 2.5e+5 | 1.0e+7 | saline | — |
| | | Wasabi- | cells/mouse | | | |
| | | ffLuc | | | | |
| 26 | 5 | | — | 1.0e+7 | — | — |
| 27 | 5 | Nalm6: | 2.5e+5 | 1.0e+7 | Saline | |
| | | Wasabi- | cells/mouse | | | |
| | | ffLuc | | | | |
| 28 | 5 | | — | 1.0e+7 | — | — |

TABLE 35
CD19 CAR sequences

| | | SEQ |
|---|---|---|
| CD19 | | ID |
| CAR | VH Sequence | NO: |
| 1 (369 | EVQLVESGGGLIQPGGSLRLSCAASGFTVSSNYMSWVRQAPGKGLEWVSVIYSG | 232 |
| VH-VL) | GSTYYADSVKGRFTISRDNSKNTLYLQMNSLRAEDTAVYYCARDGGLDTLYGMD | |
| AA | VWGQGTTVTVSSGGGGSGGGGSGGGGSQSVVTQPPSVSGAPGQRVTISCTGS | |
| | SSNIGAGYDVHWYQQLPGTAPKLLIYGNSNRPSGVPDRFSGSKSGTSASLAITGL | |
| | QAEDEADYYCQSYDSSLSGSYVFGTGTKVTVLTTTPAPRPPTPÅPTIASQPLSLRP | |
| | EACRPAAGGAVHTRGLDFACDIYIWAPLAGTCGVLLLSLVITLYCKRGRKKLLYIFK | |
| | QPFMRPVQTTQEEDGCSCRFPEEEEGGCELRVKFSRSADAPAYQQGQNQLYNE | |
| | LNLGRREEYDVLDKRRGRDPEMGGKPRRKNPQEGLYNELQKDKMAEAYSEIGM | |
| | KGERRRGKGHDGLYQGLSTATKDTYDALHMQALPPR | |
| 1 (369 | GAAGTGCAGCTGGTGGAGTCTGGAGGAGGCTTGATCCAGCCTGGGGGGTCC | 233 |
| VH-VL) | CTGAGACTCTCCTGTGCAGCCTCTGGGTTCACCGTCAGTAGCAACTACATGAG | |
| Nt | CTGGGTTCGCCAGGCTCCAGGGAAGGGGCTGGAGTGGGTCTCÅGTTATTTAT | |
| | AGCGGTGGTAGCACATACTACGCAGACTCCGTGAAGGGCCGATTCACCATCT | |
| | CCAGAGACAATTCCAAGAACACGCTGTATCTTCAAATGAACAGCCTGAGAGC | |
| | CGAGGACACGGCCGTGTATTACTGTGCGAGAGATGGAGGTCTCGATACTCTC | |
| | TACGGTATGGACGTCTGGGGCCAAGGGACCACGGTCACCGTCTCCTCAGGTG | |
| | GAGGCGGTTCCGGAGGCGGTGGCAGCGGCGGTGGCGGTTCGCAGTCTGTC | |
| | GTGACGCAGCCGCCCTCAGTGTCTGGGGCCCCAGGGCAGAGGGTCACCATC | |
| | TCCTGCACTGGGAGCAGCTCCAACATCGGGGCAGGTTATGATGTACACTGGT | |
| | ACCAGCAGCTTCCAGGAACAGCCCCCAAACTCCTCATCTATGGTAACAGCAAT | |
| | CGGCCCTCAGGGGTCCCTGACCGATTCTCTGGCTCCAAGTCTGGCACCTCAGC | |
| | CTCCCTGGCCATCACTGGGCTCCAGGCTGAGGATGAGGCTGATTATTACTGC | |
| | CAGTCCTATGACAGCAGCCTGAGTGGTTCGTATGTCTTCGGAACTGGGACCA | |
| | AGGTGACCGTCCTTaccactacaccagcccctaggcctccaacaccggcgcccaccatcgcgtcg | |
| | cagcccctgtccctgcgcccagaggcgtgccggccagcggggggggcgcagtgcacacgagggggct | |
| | ggacttcgcctgtgatatctacatctgggcgcccttggccgggacttgtggggtccttctcctgtca | |
| | ctggttatcaccctttactgcaaacggggcagaaagaaactcctgtatatattcaaacaaccattta | |
| | tgagaccagtacaaactactcaagaggaagatggctgtagctgccgatttccagaagaagaagaagg | |
| | aggatgtgaactgagagtgaagttcagcaggagcgcagacgcccccgcgtaccagcagggccagaac | |
| | cagctctataacgagctcaatctaggacgaagagaggagtacgatgttttggacaagagacgtggcc | |
| | gggaccctgagatggggggaaagccgagaaggaagaaccctcaggaaggcctgtacaatgaactg | |
| | cagaaagataagatggcggaggcctacagtgagattgggatgaaaggcgagcgccggaggggcaa | |
| | ggggcacgatggcctttaccagggtctcagtacagccaccaaggacacctacgacgcccttcacatg | |
| | caggccctgccccctcgc | |
| 2 (399 | EVHLVESGGGLVQPGGSLRLSCAASGFTFSDYYMSWIRQAPGKGLEWVSYI | 234 |
| VH-VL) | SSSGSTTYYADSVKGRFTISRDNAKNSLYLQMNSLRAEDTAVYYCARVEGW | |
| AA | SLLDYWGQGTLVTVSSGGGGGGGGSGGGGSQSVLTQPPSVSAAPGQKV | |
| | TMSCSGSSSNIGNNYVSWYQQLPGTAPKLLIYDNNKRPSGIPDRFSGSKSG | |
| | TSATLGITGLQTGDEADYYCGTWDSSLGÅVVFGGGTKVTVLTTTPAPRPPT | |
| | PAPTIASQPLSLRPEACRPAAGGAVHTRGLDFACDIYIWAPLAGTCGVLLLSL | |
| | VITLYCKRGRKKLLYIFKQPFMRPVQTTQEEDGCSCRFPEEEEGGCELRVKFS | |
| | RSADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTYDAL | |
| | HMQALPPR | |
| 2 (399 | GAGGTGCATCTGGTGGAGTCTGGGGGAGGCTTGGTACAGCCGGGGGG | 235 |
| VH-VL) | GTCCCTGAGACTCTCCTGTGCAGCCTCTGGATTCACCTTCAGTGACTACT | |
| Nt | ACATGAGCTGGATCCGCCAGGCTCCAGGGAAGGGGCTGGAGTGGGTTT | |
| | CATACATTAGTAGTAGTGGTAGTACCACATACTACGCAGACTCCGTGAA | |
| | GGGCCGGTTCACCATCTCCAGAGACAACGCCAAGAACTCACTGTATCTG | |
| | CAAATGAACAGCCTGAGAGCCGAGGACACGGCCGTGTATTACTGTGCG | |
| | AGAGTAGAAGGCTGGAGTCTTTTGGACTACTGGGGCCAGGGAACCCTG | |
| | GTCACTGTCTCCTCAGGTGGAGGCGGTTCCGGAGGCGGTGGCAGCGGC | |
| | GGTGGCGGTTCGCAGTCTGTGTTGACGCAGCCGCCCTCAGTGTCTGCGG | |
| | CCCCAGGACAGAAGGTCACCATGTCCTGCTCTGGAAGCAGCTCCAACAT | |
| | TGGGAATAATTATGTATCCTGGTACCAGCAGCTCCCAGGAACAGCCCCC | |
| | AAACTCCTCATTTATGACAATAATAAGCGACCCTCAGGGATTCCTGACCG | |
| | ATTCTCTGGCTCCAAGTCTGGCACGTCAGCCACCCTGGGCATCACCGGA | |
| | CTCCAGACTGGGGACGAGGCCGATTATTATTGCGGAACATGGGATAGT | |
| | AGCCTGGGTGCTGTGGTATTCGGCGGGGGGACCAAGGTGACCGTCCTC | |
| | accactacaccagcccctaggcctccaacaccggcgcccaccatcgcgtcgcagcccctgtccc | |
| | tgcgcccagaggcgtgccggccagcggggggggcgcagtgcacacgagggggctggacttc | |
| | gcctgtgatatctacatctgggcgcccttggccgggacttgtggggtccttctcctgtcactggtt | |
| | atcaccctttactgcaaacggggcagaaagaaactcctgtatatattcaaacaaccatttatga | |
| | gaccagtacaaactactcaagaggaagatggctgtagctgccgatttccagaagaagaagaa | |
| | ggaggatgtgaactgagagtgaagttcagcaggagcgcagacgcccccgcgtaccagcaggg | |
| | ccagaaccagctctataacgagctcaatctaggacgaagagaggagtacgatgttttggacaa | |
| | gagacgtggccgggaccctgagatggggggaaagccgagaaggaagaaccctcaggaaggc | |
| | ctgtacaatgaactgcagaaagataagatggcggaggcctacagtgagattgggatgaaagg | |
| | cgagcgccggaggggcaaggggcacgatggcctttaccagggtctcagtacagccaccaagg | |
| | acacctacgacgcccttcacatgcaggccctgccccctcgc | |
| 3 (400 | EVQLVQSGAEVKKPGESLKISCKGSGYSFTSYWIGWVRQMPGKGLEWMGI | 236 |
| VH-VL) | IYPGDSDTRYSPSFQGQVTISADKSISTAYLQWSSLKASDTAMYYCARLPFV | |
| AA | GATTGGFDYWGQGTLVTVSSGGGGSGGGGSGGGGSQSVVTQPASVSGS | |
| | PGQSITISCTGTSSDVGGYNYVSWYQQHPGKAPKLVIYEVSNRPSGVSNRFS | |
| | GSKSGNTASLTISGLQAEDEADYYCSSYTSSSTLVFGGGTKLTVLTTTPAPRP | |
| | PTPAPTIASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEEGGCELRV | |
| | KFSRSADAPAYQQGQNQLYNELNLGRREEYDVLDKRRGRDPEMGGKPRR | |
| | KNPQEGLYNELQKDKMAEAYSEIGMKGERRRGKGHDGLYQGLSTATKDTY | |
| | DALHMQALPPR | |
| 3 (400 | GAAGTGCAGCTGGTGCAGTCTGGAGCAGAGGTGAAAAAGCCCGGGGA | 237 |
| VH-VL) | GTCTCTGAAGATCTCCTGTAAGGGTTCTGGATACAGCTTTACCAGCTACT | |
| | GGATCGGCTGGGTGCGCCAGATGCCCGGGAAAGGCCTGGAGTGGATG | |
| | GGGATCATCTATCCTGGTGACTCTGATACCAGATACAGCCCGTCCTTCCA | |
| | AGGCCAGGTCACCATCTCAGCCGACAAGTCCATCAGCACCGCCTACCTG | |
| | CAGTGGAGCAGCCTGAAGGCCTCGGACACCGCCATGTATTACTGTGCGA | |
| | GACTTCCTTTTGTGGGAGCTACCACGGGGGGCTTTGACTACTGGGGCCA | |
| | GGGAACCCTGGTCACCGTCTCCTCAGGTGGAGGCGGTTCCGGAGGCGG | |
| | TGGCAGCGGCGGTGGCGGTTCGCAGTCTGTCGTGACGCAGCCTGCCTCC | |
| | GTGTCTGGGTCTCCTGGACAGTCGATCACCATCTCCTGCACTGGAACCA | |
| | GCAGTGACGTTGGTGGTTATAACTATGTCTCCTGGTACCAACAGCACCC | |
| | AGGCAAAGCCCCCAAACTCGTGATTTATGAGGTCAGTAATCGGCCCTCA | |
| | GGGGTTTCTAATCGCTTCTCTGGCTCCAAGTCTGGCAACACGGCCTCCCT | |
| | GACCATCTCTGGGCTCCAGGCTGAGGACGAGGCTGATTATTACTGCAGC | |
| | TCATATACAAGCAGCAGCACTCTGGTGTTCGGCGGAGGGACCAAGCTG | |
| | ACCGTCCTAaccactacaccagcccctaggcctccaacaccggcgcccaccatcgcgtcgc | |
| | agcccctgtccctgcgcccagaggcgtgccggccagcggggggggcgcagtgcacacgagg | |
| | gggctggacttcgcctgtgatatctacatctgggcgcccttggccgggacttgtggggtccttctc | |
| | ctgtcactggttatcaccctttactgcaaacggggcagaaagaaactcctgtatatattcaaaca | |
| | accatttatgagaccagtacaaactactcaagaggaagatggctgtagctgccgatttccaga | |
| | agaagaagaaggaggatgtgaactgagagtgaagttcagcaggagcgcagacgcccccgcg | |
| | taccagcagggccagaaccagctctataacgagctcaatctaggacgaagagaggagtacga | |
| | tgttttggacaagagacgtggccgggaccctgagatggggggaaagccgagaaggaagaac | |
| | cctcaggaaggcctgtacaatgaactgcagaaagataagatggcggaggcctacagtgagat | |
| | tgggatgaaaggcgagcgccggaggggcaaggggcacgatggcctttaccagggtctcagta | |
| | cagccaccaaggacacctacgacgcccttcacatgcaggccctgccccctcgc | |
| 1 (369 | QSVVTQPPSVSGAPGQRVTISCTGSSSNIGAGYDVHWYQQLPGTAPKLLIY | 238 |
| VL-VH) | GNSNRPSGVPDRFSGSKSGTSASLAITGLQAEDEADYYCQSYDSSLSGSYVF | |
| AA | GTGTKVTVLGGGGSGGGGSGGGGSEVQLVESGGGLIQPGGSLRLSCAASG | |
| | FTVSSNYMSWVRQAPGKGLEWVSVIYSGGSTYYADSVKGRFTISRDNSKNT | |
| | LYLQMNSLRAEDTAVYYCARDGGLDTLYGMDVWGQGTTVTVSSTTTPAP | |
| | RPPTPAPTIASQPLSLRPEACRPAAGGAVHTRGLDFACDIYIWAPLAGTCGV | |
| | LLLSLVITLYCKRGRKKLLYIFKQPFMRPVQTTQEEDGCSCRFPEEEEGGCELR | |
| | VKFSRSADAPAYQQGQNQLYNELNLGRREEYDVLDKRRGRDPEMGGKPR | |
| | RKNPQEGLYNELQKDKMAEAYSEIGMKGERRRGKGHDGLYQGLSTATKDT | |
| | YDALHMQALPPR | |
| 1 (369 | CAGTCTGTCGTGACGCAGCCGCCCTCAGTGTCTGGGGCCCCAGGGCAG | 239 |
| VL-VH) | AGGGTCACCATCTCCTGCACTGGGAGCAGCTCCAACATCGGGGCAGGTT | |
| Nt | ATGATGTACACTGGTACCAGCAGCTTCCAGGAACAGCCCCCAAACTCCT | |
| | CATCTATGGTAACAGCAATCGGCCCTCAGGGGTCCCTGACCGATTCTCT | |
| | GGCTCCAAGTCTGGCACCTCAGCCTCCCTGGCCATCACTGGGCTCCAGG | |
| | CTGAGGATGAGGCTGATTATTACTGCCAGTCCTATGACAGCAGCCTGAG | |
| | TGGTTCGTATGTCTTCGGAACTGGGACCAAGGTGACCGTCCTTGGTGGA | |
| | GGCGGTTCCGGAGGCGGTGGCAGCGGCGGTGGCGGTTCGGAAGTGCA | |
| | GCTGGTGGAGTCTGGAGGAGGCTTGATCCAGCCTGGGGGGTCCCTGAG | |
| | ACTCTCCTGTGCAGCCTCTGGGTTCACCGTCAGTAGCAACTACATGAGCT | |
| | GGGTTCGCCAGGCTCCAGGGAAGGGGCTGGAGTGGGTCTCAGTTATTT | |
| | ATAGCGGTGGTAGCACATACTACGCAGACTCCGTGAAGGGCCGATTCAC | |
| | CATCTCCAGAGACAATTCCAAGAACACGCTGTATCTTCAAATGAACAGCC | |
| | TGAGAGCCGAGGACACGGCCGTGTATTACTGTGCGAGAGATGGAGGTC | |
| | TCGATACTCTCTACGGTATGGACGTCTGGGGCCAAGGGACCACGGTCAC | |
| | CGTCTCCTCAaccactacaccagcccctaggcctccaacaccggcgcccaccatcgcgtcg | |
| | cagcccctgtccctgcgcccagaggcgtgccggccagcggggggggcgcagtgcacacgag | |
| | ggggctggacttcgcctgtgatatctacatctgggcgcccttggccgggacttgtggggtccttct | |
| | cctgtcactggttatcaccctttactgcaaacggggcagaaagaaactcctgtatatattcaaac | |
| | aaccatttatgagaccagtacaaactactcaagaggaagatggctgtagctgccgatttccag | |
| | aagaagaagaaggaggatgtgaactgagagtgaagttcagcaggagcgcagacgcccccgc | |
| | gtaccagcagggccagaaccagctctataacgagctcaatctaggacgaagagaggagtacg | |
| | atgttttggacaagagacgtggccgggaccctgagatggggggaaagccgagaaggaagaa | |
| | ccctcaggaaggcctgtacaatgaactgcagaaagataagatggcggaggcctacagtgaga | |
| | ttgggatgaaaggcgagcgccggaggggcaaggggcacgatggcctttaccagggtctcagt | |
| | acagccaccaaggacacctacgacgcccttcacatgcaggccctgccccctcgc | |
| 2 (399 | QSVLTQPPSVSAAPGQKVTMSCSGSSSNIGNNYVSWYQQLPGTAPKLLIYD | 240 |
| VL-VH) | NNKRPSGIPDRFSGSKSGTSATLGITGLQTGDEADYYCGTWDSSLGAVVFG | |
| AA | GGTKVTVLGGGGSGGGGSGGGGSEVHLVESGGGLVQPGGSLRLSCAASG | |
| | FTFSDYYMSWIRQAPGKGLEWVSYISSSGSTTYYADSVKGRFTISRDNAKNS | |
| | LYLQMNSLRAEDTAVYYCARVEGWSLLDYWGQGTLVTVSSTTTPAPRPPT | |
| | PAPTIASQPLSLRPEACRPAAGGAVHTRGLDFACDIYIWAPLAGTCGVLLLSL | |
| | VITLYCKRGRKKLLYIFKQPFMRPVQTTQEEDGCSCRFPEEEEGGCELRVKFS | |
| | RSADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTYDAL | |
| | HMQALPPR | |
| 2 (399 | CAGTCTGTGTTGACGCAGCCGCCCTCAGTGTCTGCGGCCCCAGGACAGA | 241 |
| VL-VH) | AGGTCACCATGTCCTGCTCTGGAAGCAGCTCCAACATTGGGAATAATTA | |
| Nt | TGTATCCTGGTACCAGCAGCTCCCAGGAACAGCCCCCAAACTCCTCATTT | |
| | ATGACAATAATAAGCGACCCTCAGGGATTCCTGACCGATTCTCTGGCTCC | |
| | AAGTCTGGCACGTCAGCCACCCTGGGCATCACCGGACTCCAGACTGGG | |
| | GACGAGGCCGATTATTATTGCGGAACATGGGATAGTAGCCTGGGTGCT | |
| | GTGGTATTCGGCGGGGGGACCAAGGTGACCGTCCTCGGTGGAGGCGGT | |
| | TCCGGAGGCGGTGGCAGCGGCGGTGGCGGTTCGGAGGTGCATCTGGT | |
| | GGAGTCTGGGGGAGGCTTGGTACAGCCGGGGGGGTCCCTGAGACTCTC | |
| | CTGTGCAGCCTCTGGATTCACCTTCAGTGACTACTACATGAGCTGGATCC | |
| | GCCAGGCTCCAGGGAAGGGGCTGGAGTGGGTTTCATACATTAGTAGTA | |
| | GTGGTAGTACCACATACTACGCAGACTCCGTGAAGGGCCGGTTCACCAT | |
| | CTCCAGAGACAACGCCAAGAACTCACTGTATCTGCAAATGAACAGCCTG | |
| | AGAGCCGAGGACACGGCCGTGTATTACTGTGCGAGAGTAGAAGGCTGG | |
| | AGTCTTTTGGACTACTGGGGCCAGGGAACCCTGGTCACTGTCTCCTCAac | |
| | cactacaccagcccctaggcctccaacaccggcgcccaccatcgcgtcgcagcccctgtccctg | |
| | cgcccagaggcgtgccggccagcggggggggcgcagtgcacacgagggggctggacttcgc | |
| | ctgtgatatctacatctgggcgcccttggccgggacttgtggggtccttctcctgtcactggttatc | |
| | accctttactgcaaacggggcagaaagaaactcctgtatatattcaaacaaccatttatgagac | |
| | cagtacaaactactcaagaggaagatggctgtagctgccgatttccagaagaagaagaagga | |
| | ggatgtgaactgagagtgaagttcagcaggagcgcagacgcccccgcgtaccagcagggcca | |
| | gaaccagctctataacgagctcaatctaggacgaagagaggagtacgatgttttggacaagag | |
| | acgtggccgggaccctgagatggggggaaagccgagaaggaagaaccctcaggaaggcctg | |
| | tacaatgaactgcagaaagataagatggcggaggcctacagtgagattgggatgaaaggcga | |
| | gcgccggaggggcaaggggcacgatggcctttaccagggtctcagtacagccaccaaggaca | |
| | cctacgacgcccttcacatgcaggccctgccccctcgc | |
| 3 (400 | QSVVTQPASVSGSPGQSITISCTGTSSDVGGYNYVSWYQQHPGKAPKLVIY | 242 |
| VL-VH) | EVSNRPSGVSNRFSGSKSGNTÅSLTISGLQAEDEADYYCSSYTSSSTLVFGGG | |
| AA | TKLTVLGGGGGGGGSGGGGSEVQLVQSGAEVKKPGESLKISCKGSGYSFT | |
| | SYWIGWVRQMPGKGLEWMGIIYPGDSDTRYSPSFQGQVTISADKSISTAYL | |
| | QWSSLKASDTAMYYCARLPFVGATTGGFDYWGQGTLVTVSSTTTPAPRPP | |
| | TPAPTIASQPLSLRPEACRPAAGGAVHTRGLDFACDIYIWAPLAGTCGVLLLS | |
| | LVITLYCKRGRKKLLYIFKQPFMRPVQTTQEEDGCSCRFPEEEEGGCELRVKF | |
| | SRSADAPAYQQGQNQLYNELNLGRREEYDVLDKRRGRDPEMGGKPRRKN | |
| | PQEGLYNELQKDKMAEAYSEIGMKGERRRGKGHDGLYQGLSTATKDTYDA | |
| | LHMQALPPR | |
| 3 (400 | CAGTCTGTCGTGACGCAGCCTGCCTCCGTGTCTGGGTCTCCTGGACAGT | 243 |
| VL-VH) | CGATCACCATCTCCTGCACTGGAACCAGTAGTGACGTTGGTGGTTATAA | |
| | CTATGTCTCCTGGTACCAACAGCACCCAGGCAAAGCCCCCAAACTCGTG | |
| | ATTTATGAGGTCAGTAATCGGCCCTCAGGGGTTTCTAATCGCTTCTCTGG | |
| | CTCCAAGTCTGGCAACACGGCCTCCCTGACCATCTCTGGGCTCCAGGCT | |
| | GAGGACGAGGCTGATTATTACTGCAGCTCATATACAAGCAGCAGCACTC | |
| | TGGTGTTCGGCGGAGGGACCAAGCTGACCGTCCTAGGTGGAGGCGGTT | |
| | CCGGAGGCGGTGGCAGCGGCGGTGGCGGTTCGGAAGTGCAGCTGGTG | |
| | CAGTCTGGAGCAGAGGTGAAAAAGCCCGGGGAGTCTCTGAAGATCTCC | |
| | TGTAAGGGTTCTGGATACAGCTTTACCAGCTACTGGATCGGCTGGGTGC | |
| | GCCAGATGCCCGGGAAAGGCCTGGAGTGGATGGGGATCATCTATCCTG | |
| | GTGACTCTGATACCAGATACAGCCCGTCCTTCCAAGGCCAGGTCACCATC | |
| | TCAGCCGACAAGTCCATCAGCACCGCCTACCTGCAGTGGAGCAGCCTGA | |
| | AGGCCTCGGACACCGCCATGTATTACTGTGCGAGACTTCCTTTTGTGGG | |
| | AGCTACCACGGGGGGCTTTGACTACTGGGGCCAGGGAACCCTGGTCAC | |
| | TGTCTCCTCAaccactacaccagcccctaggcctccaacaccggcgcccaccatcgcgtcg | |
| | cagcccctgtccctgcgcccagaggcgtgccggccagcggggggggcgcagtgcacacgag | |
| | ggggctggacttcgcctgtgatatctacatctgggcgcccttggccgggacttgtggggtccttct | |
| | cctgtcactggttatcaccctttactgcaaacggggcagaaagaaactcctgtatatattcaaac | |
| | aaccatttatgagaccagtacaaactactcaagaggaagatggctgtagctgccgatttccag | |
| | aagaagaagaaggaggatgtgaactgagagtgaagttcagcaggagcgcagacgcccccgc | |
| | gtaccagcagggccagaaccagctctataacgagctcaatctaggacgaagagaggagtacg | |
| | atgttttggacaagagacgtggccgggaccctgagatggggggaaagccgagaaggaagaa | |
| | ccctcaggaaggcctgtacaatgaactgcagaaagataagatggcggaggcctacagtgaga | |
| | ttgggatgaaaggcgagcgccggaggggcaaggggcacgatggcctttaccagggtctcagt | |
| | acagccaccaaggacacctacgacgcccttcacatgcaggccctgccccctcgc | |

TABLE 36
Experimental Groups in Example 6.

| | | | | | Effector |
|---|---|---|---|---|---|
| | | Tumor | | | Cells/ |
| Group | Target Cell | dose | Donor | Effector Cells | Mouse |
| 1. | Nalm6: | 2.5e+5 | 1 | CD47-CAR400 | 4.0e+6 |
| | Wasabi-ffLuc | cells/mouse | | (VL-VH) | |
| 2 | Nalm6: | 2.5e+5 | 1 | CD47-CAR400 | 1.0e+6 |
| | Wasabi-ffLuc | cells/mouse | | (VL-VH) | |
| 3 | Nalm6: | 2.5e+5 | 1 | CD47-CAR400 | 4.0e+6 |
| | Wasabi-ffLuc | cells/mouse | | (VH-VL) | |
| 4 | Nalm6: | 2.5e+5 | 1 | CD47-CAR400 | 1.0e+6 |
| | Wasabi-ffLuc | cells/mouse | | (VH-VL) | |
| 5 | Nalm6: | 2.5e+5 | 1 | CD47-FMC63 | 4.0e+6 |
| | Wasabi-ffLuc | cells/mouse | | | |
| 6 | Nalm6: | 2.5e+5 | 1 | CD47-FMC63 | 1.0e+6 |
| | Wasabi-ffLuc | cells/mouse | | | |
| 7 | Nalm6: | 2.5e+5 | 2 | CD47-CAR400 | 4.0e+6 |
| | Wasabi-ffLuc | cells/mouse | | (VL-VH) | |
| 8 | Nalm6: | 2.5e+5 | 2 | CD47-CAR400 | 1.0e+6 |
| | Wasabi-ffLuc | cells/mouse | | (VL-VH) | |
| 9 | Nalm6: | 2.5e+5 | 2 | CD47-CAR400 | 4.0e+6 |
| | Wasabi-ffLuc | cells/mouse | | (VH-VL) | |
| 10 | Nalm6: | 2.5e+5 | 2 | CD47-CAR400 | 1.0e+6 |
| | Wasabi-ffLuc | cells/mouse | | (VH-VL) | |
| 11 | Nalm6: | 2.5e+5 | 2 | CD47-FMC63 | 4.0e+6 |
| | Wasabi-ffLuc | cells/mouse | | | |
| 12 | Nalm6: | 2.5e+5 | 2 | CD47-FMC63 | 1.0e+6 |
| | Wasabi-ffLuc | cells/mouse | | | |
| 13 | Nalm6: | 2.5e+5 | 3 | CD47-CAR400 | 4.0e+6 |
| | Wasabi-ffLuc | cells/mouse | | (VL-VH) | |
| 14 | Nalm6: | 2.5e+5 | 3 | CD47-CAR400 | 1.0e+6 |
| | Wasabi-ffLuc | cells/mouse | | (VL-VH) | |
| 15 | Nalm6: | 2.5e+5 | 3 | CD47-CAR400 | 4.0e+6 |
| | Wasabi-ffLuc | cells/mouse | | (VH-VL) | |
| 16 | Nalm6: | 2.5e+5 | 3 | CD47-CAR400 | 1.0e+6 |
| | Wasabi-ffLuc | cells/mouse | | (VH-VL) | |
| 17 | Nalm6: | 2.5e+5 | 3 | CD47-FMC63 | 4.0e+6 |
| | Wasabi-ffLuc | cells/mouse | | | |
| 18 | Nalm6: | 2.5e+5 | 3 | CD47-FMC63 | 1.0e+6 |
| | Wasabi-ffLuc | cells/mouse | | | |
| 19 | Nalm6: | 2.5e+5 | 1 | mock | 4.0e+6 |
| | Wasabi-ffLuc | cells/mouse | | | |
| 20 | Nalm6: | 2.5e+5 | 2 | mock | 4.0e+6 |
| | Wasabi-ffLuc | cells/mouse | | | |
| 21 | Nalm6: | 2.5e+5 | 3 | mock | 4.0e+6 |
| | Wasabi-ffLuc | cells/mouse | | | |
| 22 | Naim6: | 2.5e+5 | — | — | — |
| | Wasabi-ffLuc | cells/mouse | | | |

1. An isolated polypeptide comprising an amino acid sequence selected from SEQ ID NOs: 19-21.

2. An isolated polypeptide comprising an amino acid sequence having at least 80%, 85%, 90%, 95%, 96%, 97%, 98%, or 99% identity to a sequence selected from SEQ ID NOs: 19-21.

3. An isolated polypeptide comprising an amino acid sequence selected from SEQ ID NOs: 22-24.

4. An isolated polypeptide comprising an amino acid sequence having at least 80%, 85%, 90%, 95%, 96%, 97%, 98%, or 99% identity to a sequence selected from SEQ ID NOs: 22-24.

5. An isolated protein, comprising the isolated polypeptide of claim 1 and the isolated polypeptide of claim 3.

6. An isolated protein, comprising the isolated polypeptide of claim 2 and the isolated polypeptide of claim 4.

7. An isolated polypeptide comprising an amino acid sequence selected from:
a) SEQ ID NOs: 1, 4, and 7;
b) SEQ ID NOs: 2, 5, and 8; and
c) SEQ ID NOs: 3, 6, and 9.

8. An isolated polypeptide comprising an amino acid sequence having at least 80%, 85%, 90%, 95%, 96%, 97%, 98%, or 99% identity to:
a) SEQ ID NOs: 1, 4, and 7;
b) SEQ ID NOs: 2, 4, and 8; or
c) SEQ ID NOs: 3, 6, and 9.

9. An isolated polypeptide comprising an amino acid sequence selected from:
a) SEQ ID NOs: 10, 13, and 16;
b) SEQ ID NOs: 11, 14, and 17; or
c) SEQ ID NOs: 12, 15, and 18.

10. An isolated polypeptide comprising an amino acid sequence having at least 80%, 85%, 90%, 95%, 96%, 97%, 98%, or 99% identity to:
a) SEQ ID NOs: 10, 13, and 16;
b) SEQ ID NOs: 11, 14, and 17; or
S c) SEQ ID NOs: 12, 15, and 18.

11. An isolated protein, comprising the isolated polypeptide of claim 7 and the isolated polypeptide of claim 9.

12. An isolated protein, comprising the isolated polypeptide of claim 8 and the isolated polypeptide of claim 10.

13. The isolated polypeptide or protein of any one of claims 1-12, wherein the isolated polypeptide or protein is an antibody or an antigen binding fragment thereof.

14. An antibody or antigen binding fragment thereof that specifically binds human Cluster of Differentiation 19 (CD19), comprising a heavy chain variable region (VH) comprising an amino acid sequence selected from SEQ ID NOs: 28-32.

15. An antibody or antigen binding fragment thereof that specifically binds human Cluster of Differentiation 19 (CD19), comprising a heavy chain variable region (VH) comprising an amino acid sequence having at least 80%, 85%, 90%, 95%, 96%, 97%, 98%, or 99% identity to a sequence selected from SEQ ID NOs: 19-21.

16. An antibody or antigen binding fragment thereof that specifically binds human Cluster of Differentiation 19 (CD19), comprising a light chain variable region (VL) comprising an amino acid sequence selected from SEQ ID NOs: 22-24.

17. An antibody or antigen binding fragment thereof that specifically binds human Cluster of Differentiation 19 (CD19), comprising a heavy chain variable region (VL) comprising an amino acid sequence having at least 80%, 85%, 90%, 95%, 96%, 97%, 98%, or 99% identity to a sequence selected from SEQ ID NOs: 22-24.

18. An antibody or antigen binding fragment thereof that specifically binds human Cluster of Differentiation 19 (CD19), comprising i) the heavy chain variable region (VH) of claim 14, and ii) the light chain variable region (VL) of claim 16.

19. An antibody or antigen binding fragment thereof that specifically binds human Cluster of Differentiation 19 (CD19), comprising i) the heavy chain variable region (VH) of claim 15, and ii) the light chain variable region (VL) of claim 17.

20. An antibody or antigen binding fragment thereof that specifically binds human Cluster of Differentiation 19 (CD19), comprising a heavy chain variable region (VH) comprising the sequence of SEQ ID NO: 19, and a light chain variable region (VL) comprising the sequence of SEQ ID NO: 22.

21. An antibody or antigen binding fragment thereof that specifically binds human Cluster of Differentiation 19 (CD19), comprising a heavy chain variable region (VH) comprising the sequence of SEQ ID NO: 20, and a light chain variable region (VL) comprising the sequence of SEQ ID NO: 23.

22. An antibody or antigen binding fragment thereof that specifically binds human Cluster of Differentiation 19 (CD19), comprising a heavy chain variable region (VH) comprising the sequence of SEQ ID NO: 21, and a light chain variable region (VL) comprising the sequence of SEQ ID NO: 24.

23. An antibody or antigen binding fragment thereof that specifically binds human Cluster of Differentiation 19 (CD19), comprising three heavy chain complementarity determining regions (HCDR1, HCDR2, and HCDR3) and three light chain complementarity determining regions (LCDR1, LCDR2, and LCDR3), wherein HCDR1, HCDR2, HCDR3, LCDR1, LCDR2, and LCDR3, respectively, comprise SEQ 1D NOs: 1, 4, 7, 10, 13, and 16.

24. An antibody or antigen binding fragment thereof that specifically binds human Cluster of Differentiation 19 (CD19), comprising three heavy chain complementarity determining regions (HCDR1, HCDR2, and HCDR3) and three light chain complementarity determining regions (LCDR1, LCDR2, and LCDR3), wherein HCDR1, HCDR2, HCDR3, LCDR1, LCDR2, and LCDR3, respectively, comprise SEQ ID NOs: 2, 5, 8, 11, 14, and 17.

25. An antibody or antigen binding fragment thereof that specifically binds human Cluster of Differentiation 19 (CD19), comprising three heavy chain complementarity determining regions (HCDR1, HCDR2, and HCDR3) and three light chain 4% complementarity determining regions (LCDR1, LCDR2, and LCDR3), wherein HCDR1, HCDR2, HCDR3, LCDR1, LCDR2, and LCDR3, respectively, comprise SEQ ID NOs: 3, 6, 9, 12, 15, and 18.

26. The antibody or antigen binding fragment thereof of any one of claims 14-25, wherein the antibody or antigen binding fragment thereof is a Fab, Fab′, F(ab′)2, Fd, scFv, (scFv)2, scFv-Fc, sdAb, VHH, or Fv fragment.

27. The antibody or antigen binding fragment thereof of claim 14-26, wherein the antibody or antigen binding fragment thereof is a scFv.

28. The antibody or antigen binding fragment thereof of claim 14-27, wherein the VH is N-terminal to the VL.

29. The antibody or antigen binding fragment thereof of claim 14-27, wherein the VL is N-terminal to the VH.

30. The antibody or antigen binding fragment thereof of any one of claims 26-29, wherein the scFv comprises a linker connecting the VH and VL.

31. The antibody or antigen binding fragment thereof of claim 30, wherein the linker connecting the VH and VL is a Whitlow linker.

32. The antibody or antigen binding fragment thereof of claim 30, wherein the linker connecting the VH and VL is a (G4S)3 linker (SEQ ID NO:32).

33. The antibody or antigen binding fragment of any one of claims 30-32, wherein the linker comprises an amino acid sequence selected from SEQ 10 NOs: 32-33, 145-147, and 165-166.

34. The antibody or antigen binding fragment thereof of any one of claims 14-33, wherein the antibody or antigen binding fragment thereof comprises a CD8a hinge domain.

35. The antibody or antigen binding fragment thereof of any one of claims 14-34, wherein the antibody or antigen binding fragment comprises a CD8a transmembrane domain.

36. The antibody or antigen binding fragment thereof of any one of claims 14-35, wherein the antibody or antigen binding fragment thereof binds to human CD19 with a EC₅₀ of less than 2 μg/mL.

37. A bispecific antibody or antigen binding fragment thereof, comprising the antibody or antigen binding fragment of any one of claims 14-36 and an antibody or antigen binding fragment thereof that specifically binds at least one additional cell surface molecule.

38. The bispecific antibody or antigen binding fragment thereof of claim 37, wherein the at least one additional cell surface molecule comprises CD3, 4-1BB, IL-6, NKG2D, Fc-gamma-RIIIA (CD16), APRIL, CD38, TACI, Fc-gamma-RIIIA (CD16) and NKG2D, CD3 and serum albumin, CD47 and TACI, or CD3 and GPRCSD.

39. The antibody or antigen binding fragment thereof of any one of claims 14-36 or the bispecific antibody of claim 37 or 38, wherein the antibody or antigen binding fragment comprises a conjugate.

40. An antibody or antigen binding fragment of claim 39, wherein the conjugate is a therapeutic agent, a tag for detection, a conjugate that enhances antibody stability, a nucleic acid, a cleavable linker, or a nanoparticle.

41. The antibody or antigen binding fragment thereof of any one of claims 14-36 or 39-40 or the bispecific antibody of claim 37 or 38, wherein the antibody or antigen binding fragment thereof is a monoclonal antibody.

42. The antibody or antigen binding fragment of any one of claims 14-36 or 39-41 or the bispecific antibody of claim 37 or 38, wherein the antibody or antigen binding fragment thereof is humanized.

43. An isolated polynucleotide encoding the antibody or antigen binding fragment thereof of any of claims 14-36 or 39-42 or the bispecific antibody of claim 37 or 38.

44. An isolated vector comprising the polynucleotide of claim 43.

45. The isolated vector of claim 44, wherein the vector is a polycistronic vector.

46. The isolated vector of claim 44 or 45, wherein the vector comprises nucleic acid encoding one or more additional molecules.

47. The isolated vector of claim 46, wherein the one or more additional molecules is selected from a tolerogenic factor, a suicide switch, a regulatory element, or an antibody or antigen binding fragment thereof.

48. The isolated vector of claim 46 or 47, wherein the one or more additional molecules comprises a tolerogenic factor.

49. The isolated vector of any one of claims 46-48, wherein the one or more additional molecules comprises a suicide switch.

50. The isolated vector of any one of claims 46-49, wherein the one or more additional molecules comprises a regulatory element.

51. An isolated host cell comprising the polynucleotide of claim 43, and/or the vector of any one of claims 44-50.

52. A chimeric antigen receptor (CAR) comprising an extracellular binding domain is that specifically binds human Cluster of Differentiation 19 (CD19), wherein the extracellular binding domain comprises an antigen binding domain that comprises the antibody or antigen binding fragment of any one of claims 14-36 or 39-42 or the bispecific antibody of claim 37 or 38.

53. The CAR of claim 52, wherein the CAR comprises one or more of a signal peptide, an extracellular binding domain, and a signaling domain.

54. The CAR of claim 52 or 53, wherein the CAR comprises one or more of a signal peptide, an extracellular binding domain, a hinge domain, a transmembrane domain, an intracellular costimulatory domain, and an intracellular signaling domain.

55. The CAR of any one of claims 52-54, wherein the CAR comprises a CD8a signal peptide.

56. The CAR of any one of claims 52-55, wherein the CAR comprises one or more intracellular domains selected from a CD137 (4-1BB) signaling domain, a CD28 signaling domain, and a CD3zeta signaling domain.

57. The CAR of any of claims 52-56, wherein the CAR comprises a second antigen binding domain that specifically binds CD5, CD19, C020, CD22, CD23, C030, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, C024, CD44v7/8, EGP-2, EGP-40, erb-B2, erb-B, FBP, Fetal acetylcholine e receptor, G_{D2}, G_{D3}, HMW-MAA, IL-11Rα, KDR, Lewis Y, L1-cell adhesion molecule, MADE-A1, Oncofetal antigen (h5T4), TAG-72, CD19/22, Syndecan 1, or BCMA.

58. An isolated polynucleotide encoding the CAR of any one of claims 52-57.

59. An isolated vector comprising the polynucleotide of claim 58.

60. The isolated vector of claim 59, wherein the vector is a polycistronic vector.

61. The isolated vector of claim 59 or 60, wherein the vector comprises nucleic acid encoding one or more additional molecules.

62. The isolated vector of claim 61, wherein the one or more additional molecules is selected from a tolerogenic factor, a suicide switch, a regulatory element, an antibody or antigen binding fragment thereof, or a CAR.

63. The isolated vector of claim 61 or 62, wherein the one or more additional molecules comprises a tolerogenic factor.

64. The isolated vector of any one of claims 61-63, wherein the one or more additional molecules comprises a suicide switch.

65. The isolated vector of any one of claims 61-64, wherein the one or more additional molecules comprises a regulatory element.

66. The isolated vector of any one of claims 61-65, wherein the one or more additional molecules comprises an antibody or antigen binding fragment thereof.

67. The isolated vector of any one of claims 61-66, wherein one or more additional molecules comprises a CAR.

68. A method of producing the CAR of any one of claims 52-57, comprising delivering the polynucleotide of claim 58 or the vector of any one of claims 59-67 to a host cell.

69. An isolated host cell comprising the polynucleotide of claim 58, and/or the vector of any one of claims 59-67.

70. A viral vector targeting an immune cell, the viral vector comprising:
a) an antibody or antigen binding fragment thereof that binds to a cell surface molecule on the immune cell, wherein the antibody or antigen binding fragment thereof is attached to a membrane-bound protein in the viral vector envelope or to a fusogen on the outer surface of the viral vector; and
b) at least one polynucleotide encoding the chimeric antigen receptor (CAR) of any one of claims 52-69.

71. The viral vector of claim 70, wherein the immune cell is a T cell, B cell, natural killer cell, macrophage, or monocyte.

72. The viral vector of claim 70 or 71, wherein the immune cell is a T cell.

73. The viral vector of any one of claims 70-72, wherein the antibody or antigen binding fragment thereof binds to CD3, CD4, CD7, or CD8.

74. The viral vector of any one of claims 70-73, wherein the viral vector comprises a henipavirus envelope glycoprotein G (G protein) or a biologically active portion thereof.

75. The viral vector of any one of claims 70-74, wherein the viral vector comprises a henipavirus F protein molecule or a biologically active portion thereof.

76. The viral vector of any one of claims 70-75, wherein the viral vector comprises a henipavirus envelope glycoprotein G (G protein) or a biologically active portion thereof attached to the antibody or antigen binding fragment thereof.

77. The viral vector of any one of claims 70-76, wherein the antibody or antigen binding fragment thereof binds CD8 and comprises three heavy chain complementarity determining regions (HCDR1, HCDR2, and HCDR3), and three light chain complementarity determining regions (LCDR1, LCDR2, and LCDR3), wherein HCDR1, HCDR2, HCDR3, LCDR1, LCDR2, and LCDR3, respectively, comprise:
S a) SEQ ID NOs: 78, 82, 86, 90, 94, and 98;
b) SEQ ID NOs: 79, 83, 87, 91, 95, and 99;
c) SEQ ID NOs: 80, 84, 88, 92, 96, and 100; or
d) SEQ ID NOs: 81, 85, 89, 93, 97, and 101.

78. The viral vector of any one of claims 70-77, wherein the antibody or antigen binding fragment thereof comprises a heavy chain variable region (VH) having an amino acid sequence with at least 90%, 95%, 96%, 97%, 98%, 99%, or 100% identity to a sequence selected from SEQ ID NOs: 102-105.

79. The viral vector of any one of claims 70-78, wherein the antibody or antigen binding fragment thereof comprises a light chain variable region (VL) having an amino acid sequence with at least 90%, 95%, 96%, 97%, 98%, 99%, or 100% identity to a sequence selected from SEQ ID NOs: 106-109.

80. The viral vector of any one of claims 70-79, wherein the antibody or antigen binding fragment thereof comprises a VH having an amino acid sequence with at least 90%, 95%, 96%, 97%, 98%, 99%, or 100% identity to a sequence selected from SEQ ID NOs: 102-105 and a VL having an amino acid sequence with at least 90%, 95%, 96%, 97%, 98%, 99%, or 100% identity to a sequence selected from SEQ ID NOs: 106-109.

81. The viral vector of any one of claims 70-76, wherein the antibody or antigen binding fragment thereof binds CD4, and comprises three heavy chain complementarity determining regions (HCDR1, HCDR2, and HCDR3), and three light chain complementarity determining regions (LCDR1, LCDR2, and LCDR3), wherein HCDR1, HCDR2, HCDR3, LCOR1, LCDR2, and LCDR3, respectively, comprise:
a) SEQ ID NOs: 50, 54, 58, 62, 65, and 68;
b) SEQ ID NOs: 51, 55, 59, 63, 66, and 69; or
c) SEQ ID NOs: 52, 56, 60, 64, 67, and 70;
or wherein the HCDR1, HCDR2, and HCDR3, respectively, comprise:
d) SEQ ID NOs: 53, 57, and 61.

82. The viral vector of claim 70-76 or 81, wherein the antibody or antigen binding fragment thereof comprises a heavy chain variable region (VH) having an amino acid sequence with at least 90%, 95%, 96%, 97%, 98%, 99%, or 100% identity to a sequence selected from SEQ ID NOs: 71-74.

83. The viral vector of claim 70-76 or 81-82, wherein the antibody or antigen binding fragment thereof comprises a light chain variable region (VL) having an amino acid sequence with at least 90%, 95%, 96%, 97%, 98%, 99%, or 100% identity to a sequence selected from SEQ ID NOs: 75-77.

84. The viral vector of any one of claims 70-76 or 81-83, wherein the antibody or antigen binding fragment thereof comprises a VH having an amino acid sequence with at least 90%, 95%, 96%, 97%, 98%, 99%, or 100% identity to a sequence selected from SEQ ID NOs: 71-74 and a VL having an amino acid sequence with at least 90%, 95%, 96%, 97%, 98%, 99%, or 100% identity to a sequence selected from SEQ ID NOs: 75-77.

85. The viral vector of any one of claims 70-84, wherein the G protein is a wild-type Nipah virus G glycoprotein (NiV-G) or a functionally active variant or a biologically active portion thereof.

86. The viral vector of claim 85, wherein the NiV-G variant or biologically active portion thereof comprises an amino acid sequence having at least 90%, 95%, 96%, 97%, 98%, 99%, or 100% sequence identity to SEQ ID NO: 120, SEQ ID NO: 138, or SEQ ID NO: 148.

87. The viral vector of claim 85 or 86, wherein the NiV-G variant or biologically active portion thereof comprises one or more amino acid substitutions corresponding to amino acid substitutions selected from E501A, W504A, Q530A, and E533A with reference to the numbering set forth in SEQ ID NO: 138.

88. The viral vector of any one of claims 85-87, wherein the NiV-G variant comprises SEQ ID NO: 127 or 155.

89. The viral vector of any one of claims 71-88, wherein the F protein is a wild-type Nipah virus F (NiV-F) protein or a functionally active variant or a biologically active portion thereof.

90. The viral vector of claim 89, wherein the NiV-F comprises an amino acid sequence having at least 90%, 95%, 96%, 97%, 98%, 99%, or 100% sequence identity to SEQ ID NO: 111, SEQ ID NO: 112, SEQ ID NO: 114, SEQ ID NO: 115, SEQ ID NO: 117, SEQ ID NO: 118, or SEQ ID NO: 119.

91. A fusion protein comprising a henipavirus envelope glycoprotein G (G protein) or a biologically active portion thereof and the antibody or antigen binding fragment thereof of any one of claims 14-42.

92. The fusion protein of claim 91, wherein the antibody or antigen binding fragment thereof is fused to the G protein via a peptide linker.

93. The fusion protein of claim 91 or 92, wherein the peptide linker comprises (GGGGS)n, wherein n is 3 (SEQ ID NO: 32).

94. The fusion protein of any one of claims 91-93, wherein the antigen binding fragment is a scFv.

95. The fusion protein of any one of claims 91-94, wherein the G protein or a biologically active portion thereof is a wild-type Nipah virus G glycoprotein (NiV-G) or a functionally active variant or a biologically active portion thereof.

96. The fusion protein of claim 95, wherein the NiV-G variant or biologically active portion thereof comprises an amino acid sequence having at least 90%, 95%, 96%, 97%, 98%, 99%, or 100% sequence identity to SEQ ID NO: 120, SEQ ID NO: 138, or SEQ ID NO: 148.

97. The fusion protein of claim 95 or 96, wherein the NiV-G variant or biologically active portion thereof comprises one or more amino acid substitutions corresponding to amino acid substitutions selected from E501A, W504A, Q530A and E533A with reference to the numbering set forth in SEQ ID NO: 138.

98. The fusion protein of any one of claims 95-97, wherein the NiV-G variant comprises SEQ ID NO: 127 or 155.

99. The fusion protein of any one of claims 91-98, in which the protein is pseudotyped onto a lentiviral particle.

100. A method for selectively modulating the activity of an immune cell, comprising delivering to the immune cell an effective amount of a viral vector comprising a polynucleotide encoding a chimeric antigen receptor (CAR), wherein the viral vector is the viral vector of any one of claims 70-90.

101. A method for making a CAR immune cell, comprising delivering to the immune cell an effective amount of a viral vector comprising a polynucleotide encoding a chimeric antigen receptor (CAR), wherein the viral vector is the viral vector of any one of claims 70-90.

102. The method of claim 100 or 101, wherein the immune cell is a T cell, B cell, natural killer cell, macrophage, or monocyte.

103. The method of anyone of claims 100-102, wherein the immune cell is a T cell.

104. The method of claim 102 or 103, wherein the T cell is a CD3+ T cell, a CD4+ T cell, a CDS+ T cell, a naive T cell, a regulatory T (Treg) cell, a non-regulatory T cell, a Th cell, a Th2 cell, a Th9 cell, a Th17 cell, a T-follicular helper (Tfh) cell, a cytotoxic T lymphocyte (CTL), an effector T (Teff) cell, a central memory T cell, an effector memory T cell, an effector memory T cell expressing CD45RA (TEMRA cell), a tissue-resident memory (Trm) cell, a virtual memory T cell, an innate memory T cell, a memory stem cell (Tse), or a γδ T cell.

105. The method of any one of claims 102-104, wherein the T cell is a cytotoxic T cell, a helper T cell, a memory T cell, a regulatory T cell, or a tumor infiltrating lymphocyte.

106. The method of any one of claims 102-105, wherein the T cell is a human T cell.

107. The method of any one of claims 102-106, wherein the T cell is an autologous T cell.

108. The method of anyone of claims 102-107, wherein the T cell is an allogeneic T cell.

109. The method of claim 108, wherein the allogeneic T cell is a primary T cell.

110. The method of claim 109, wherein the primary T cell has been collected from a sample comprising cells from a single donor.

111. The method of claim 109, wherein the primary T cell has been collected from a sample comprising cells multiple donors.

112. The method of anyone of claims 108-111, wherein the allogeneic T cell has been differentiated from an embryonic stem cell (ESC) or an induced pluripotent stem cell (iPSC).

113. The method of anyone of claims 100-112, wherein after delivering to the immune cell, the polynucleotide encoding the CAR is inserted into a site-specific locus.

114. The method of claim 113, wherein the site-specific locus is a safe harbor locus.

115. The method of claim 114, wherein the site-specific locus is selected from TRAC, TRBC1, TRBC2, B2M, CIITA, MICA, MICB, AAVS1, ABO, CCR5, CLYBL, CXCR4, F3, FUT1, HMGB1, KDMSD, LRP1, RHD, ROSA26, or SHS23.

116. The method of anyone of claims 113-115, wherein the polynucleotide encoding the CAR is inserted by homology-directed repair (HDR).

117. The method of claim 116, wherein the polynucleotide encoding the CAR is inserted by a CRISPR-associated transposase, prime editing, a TnpB polypeptide, or Programmable Addition via Site-specific Targeting Elements (PASTE).

118. The method of claim 116 or 117, wherein the polynucleotide encoding the CAR is inserted by a site-directed nuclease.

119. The method of claim 118, wherein the site-directed nuclease is selected from a zinc finger nuclease (ZFN), a TAL-effector nuclease (TALEN), and a CRISPR-Cas combination.

120. The method of claim 118 or 119, wherein the site-directed nuclease is selected from the group consisting of: Cas3, Cas4, Cas5, Cassa, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, and a TnpB polypeptide.

121. The method of anyone of claims 116-120, wherein the polynucleotide encoding the CAR is inserted using a guide RNA (gRNA) and a CRISPR-associated (Cas) nuclease.

122. The method of claim 121, wherein the gRNA comprises a complementary region, wherein the complementary region comprises a nucleic acid sequence that is complementary to a target nucleic acid sequence within the locus, and wherein the target nucleic acid sequence comprises an insertion site.

123. An immune cell comprising the CAR of any one of claims 52-57.

124. The immune cell of claim 123, wherein the immune cell further comprises a second CAR that specifically binds CD5, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRα, IL-13Ra, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, G_{D2}, G_{D3}, HMW-MAA, IL-11RG, KDR, Lewis Y, L1-cell adhesion molecule, MADE-A1, Oncofetal antigen (h5T4), TAG-72, CD19/22, Syndecan 1, or BCMA.

125. The cell of claim 123 or 124, wherein the cell is a T cell, B cell, natural killer cell, macrophage, or monocyte.

126. The cell of claim 125, wherein the cell is a T cell.

127. The cell of claim 125 or 126, wherein the T cell is a CD3+ T cell, a CD4+ T cell, a CDS+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T cell, an effector memory T cell, an effector memory T cell expressing CD45RA (TEMRA cell), a tissue-resident memory (Trm) cell, a virtual memory T cell, an innate memory T cell, a memory stem cell (Tse), or a yb T cell.

128. The cell of any one of claims 125-127, wherein the T cell is a cytotoxic T cell, a helper T cel, a memory T cell, a regulatory T cell, or a tumor infiltrating lymphocyte.

129. The cell of any one of claims 125-128, wherein the T cell is a human T cell.

130. The cell of any one of claims 125-129, wherein the T cell is an autologous T cell.

131. The cell of any one of claims 125-129, wherein the T cell is an allogeneic T cell.

132. The cell of claim 131, wherein the allogeneic T cell is a primary T cell.

133. The cell of claim 132, wherein the primary T cell has been collected from a sample comprising cells from a single donor.

134. The cell of claim 132, wherein the primary T cell has been collected from a sample comprising cells from multiple donors.

135. The cell of any one of claims 131-134, wherein the allogeneic T cell has been differentiated from an embryonic stem cell (ESC) or an induced pluripotent stem cell (iPSC).

136. An engineered cell comprising:
a) the CAR from any one of claims 52-57; and
b) one or more modifications that (i) reduce expression of one or more MHC class I molecules and/or one or more MHC class II molecules, and/or (ii) increase expression of one of more tolerogenic factors, wherein the reduced expression of (i) and the increase expression of (ii) is relative to a cell of the same cell type that does not comprise the modifications.

137. The engineered cell of claim 136, wherein the one or more modifications that increase expression comprise increased cell surface expression, and/or the one or more modifications that reduce expression comprise reduced cell surface expression.

138. The engineered cell of claim 136 or 137, wherein the one or more modifications in (i) reduce expression of:
a) one or more MHC class I molecules;
b) one or more MHC class II molecules; or
c) one or more MHC class 1 molecules and one or more MHC class II molecules.

139. The engineered cell of anyone of claims 138-138, wherein the one or more modifications in (i) reduce expression of one or more molecules selected from 82M, TAP I, NLRC5, CIITA, HLA-A, HLA-B, HLA-C, HLA-DP, HLA-DO, HLA-DR, HLA-DM, HLA-DO, RFX5, RFXANK, RFXAP, NFY-A, NFY-B, and NFY-C.

140. The engineered cell of claim 139, wherein the engineered cell does not express one or more molecules selected from B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, and NFY-C.

141. The engineered cell of any one of claims 136-140, wherein reduced expression comprises inactivation, disruption, or knocking out of one or both alleles of a gene encoding or regulating expression of the one or more MHC class I molecules and/or the one or more MHC class II molecules.

142. The engineered cell of any one of claims 136-141, wherein the one or more tolerogenic factors comprise one or more tolerogenic factor selected from A20/TNFAIP3, C1-Inhibitor, CCL21, CCL22, CD16, CD16 Fc receptor, CD24, CD27, CD35, CD39, CD46, CD47, CD52, CD55, CD59, CD200, CR1, CTLA4-4g, DUX4, FasL, H2-M3, HLA-C, HLA-E, HLA-E heavy chain, HLA-F, HLA-G, IDO1, IL-10, IL-15RF, IL-35, MANF, Mfge8, PD-L1, and Serpinb9.

143. The engineered cell of any one of claims 136-142, wherein increased expression comprises a modification that increases activity of a gene encoding or regulating expression of the one or more tolerogenic factors.

144. A method comprising administering to a subject in need thereof an effective amount of the CAR cells of any one of claims 123-143.

145. The method of claim 1244, wherein the method is for treating a disease in the subject.

146. A population of immune cells expressing the CARs of any one of claims 52-57 or the cells of any one of claims 123-143, for use in treating a disease in a subject.

147. A composition of immune cells expressing the CARs of any one of claims 52-57 or the cells of any one of claims 123-143, for use in treating a disease in a subject.

148. A pharmaceutical composition of immune cells expressing the CARs of any one of claims 52-57 or the cells of any one of claims 123-143 for use in treating a disease in a subject.

149. Use of the population of cells of claim 146, the composition of cells of claim 147, or the pharmaceutical composition of claim 148 for use In treating a disease in a subject.

150. Use of the population of cells of claim 146, the composition of cells of claim 147, or the pharmaceutical composition of claim 148 in the manufacture of a medicament for the treatment of a disease.

151. The method of claim 144 or 145, the population of cells of claim 146, the composition of claim 147, the pharmaceutical composition of claim 148, or the use of claim 149 or 150, wherein the disease is cancer.

152. The method, the population of cells, the composition, the pharmaceutical composition, or use of any of claims 144-151, wherein the cancer is associated with CD5, CD19, CD20, CD22, CD23, CD30, CD33, CD70, Kappa, Lambda, B cell maturation agent (BCMA), G-protein coupled receptor family C group 5 member D (GPRCSD), CD123, LeY, NKG2D ligand, WT1, GD2, HER2, EGFR, EGFRvIll, 87H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3, HPV16-E6, IL13Ra2, MAGEA3, MAGEA4, MART1, NY-ESO-1, VEGFR2, α-Folate receptor, CD24, CD44v718, EGP-2, EGP-40, erb-B2, erb-B 2,3,4, FBP, Fetal acetylcholine e receptor, G_{D2}, G_{D3}, HMW-MAA, IL-11Ra, KDR, Lewis Y, L1-cell adhesion molecule, MAGE-A1, Oncofetal antigen (h5T4), and/or TAG-72 expression.

153. The method, the population of cells, the composition, the pharmaceutical composition, or use of any of claims 144-152, wherein the cancer is a hematologic malignancy.

154. The method, the population of cells, the composition, the pharmaceutical composition, or use of any one of claims 144-153, wherein the hematologic malignancy is selected from myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), multiple myeloma (MM), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

155. The method, the population of cells, the composition, the pharmaceutical composition, or use of any one of claims 144-154, wherein the cancer is a solid malignancy.

156. The method, the population of cells, the composition, the pharmaceutical composition, or use of any one of claims 144-155, wherein the solid malignancy is selected from breast cancer, ovarian cancer, colon cancer, prostate cancer, epithelial cancer, renal-cell carcinoma, pancreatic adenocarcinoma, cervical carcinoma, colorectal cancer, glioblastoma, rhabdomyosarcoma, neuroblastoma, melanoma, Ewing sarcoma, osteosarcoma, mesothelioma, and adenocarcinoma.


SEQUENCE LISTING (243 sequences)

SEQ ID NO: 1 - protein, 5 aa, synthetic construct
SNYMS

SEQ ID NO: 2 - protein, 5 aa, synthetic construct
DYYMS

SEQ ID NO: 3 - protein, 5 aa, synthetic construct
SYWIG

SEQ ID NO: 4 - protein, 16 aa, synthetic construct
VIYSGGSTYYADSVKG

SEQ ID NO: 5 - protein, 17 aa, synthetic construct
YISSSGSTTYYADSVKG

SEQ ID NO: 6 - protein, 17 aa, synthetic construct
IIYPGDSDTRYSPSFQG

SEQ ID NO: 7 - protein, 12 aa, synthetic construct
DGGLDTLYGMDV

SEQ ID NO: 8 - protein, 9 aa, synthetic construct
VEGWSLLDY

SEQ ID NO: 9 - protein, 13 aa, synthetic construct
LPFVGATTGGFDY

SEQ ID NO: 10 - protein, 14 aa, synthetic construct
TGSSSNIGAGYDVH

SEQ ID NO: 11 - protein, 13 aa, synthetic construct
SGSSSNIGNNYVS

SEQ ID NO: 12 - protein, 14 aa, synthetic construct
TGTSSDVGGYNYVS

SEQ ID NO: 13 - protein, 7 aa, synthetic construct
GNSNRPS

SEQ ID NO: 14 - protein, 7 aa, synthetic construct
DNNKRPS

SEQ ID NO: 15 - protein, 7 aa, synthetic construct
EVSNRPS

SEQ ID NO: 16 - protein, 12 aa, synthetic construct
QSYDSSLSGSYV

SEQ ID NO: 17 - protein, 11 aa, synthetic construct
GTWDSSLGAVV

SEQ ID NO: 18 - protein, 10 aa, synthetic construct
SSYTSSSTLV

SEQ ID NO: 19 - protein, 120 aa, synthetic construct
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDGGLDTLYGMDVWGQGTTVTVSS

SEQ ID NO: 20 - protein, 118 aa, synthetic construct
EVHLVESGGGLVQPGGSLRLSCAASGFTFSDYYMSWIRQAPGKGLEWVSYISSSGSTTYYADSVKGRFTISRDNAKNSLYLQMNSLRAEDTAVYYCARVEGWSLLDYWGQGTLVTVSS

SEQ ID NO: 21 - protein, 122 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLPFVGATTGGFDYWGQGTLVTVSS

SEQ ID NO: 22 - protein, 112 aa, synthetic construct
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKVTVL

SEQ ID NO: 23 - protein, 110 aa, synthetic construct
QSVLTQPPSVSAAPGQKVTMSCSGSSSNIGNNYVSWYQQLPGTAPKLLIYDNNKRPSGIPDRFSGSKSGTSATLGITGLQTGDEADYYCGTWDSSLGAVVFGGGTKVTVL

SEQ ID NO: 24 - protein, 110 aa, synthetic construct
QSVVTQPASVSGSPGQSITISCTGTSSDVGGYNYVSWYQQHPGKAPKLVIYEVSNRPSGVSNRFSGSKSGNTASLTISGLQAEDEADYYCSSYTSSSTLVFGGGTKLTVL

SEQ ID NO: 25 - protein, 247 aa, synthetic construct
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDGGLDTLYGMDVWGQGTTVTVSSGGGGSGGGGSGGGGSQSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKVTVL

SEQ ID NO: 26 - protein, 243 aa, synthetic construct
EVHLVESGGGLVQPGGSLRLSCAASGFTFSDYYMSWIRQAPGKGLEWVSYISSSGSTTYYADSVKGRFTISRDNAKNSLYLQMNSLRAEDTAVYYCARVEGWSLLDYWGQGTLVTVSSGGGGSGGGGSGGGGSQSVLTQPPSVSAAPGQKVTMSCSGSSSNIGNNYVSWYQQLPGTAPKLLIYDNNKRPSGIPDRFSGSKSGTSATLGITGLQTGDEADYYCGTWDSSLGAVVFGGGTKVTVL

SEQ ID NO: 27 - protein, 247 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLPFVGATTGGFDYWGQGTLVTVSSGGGGSGGGGSGGGGSQSVVTQPASVSGSPGQSITISCTGTSSDVGGYNYVSWYQQHPGKAPKLVIYEVSNRPSGVSNRFSGSKSGNTASLTISGLQAEDEADYYCSSYTSSSTLVFGGGTKLTVL

SEQ ID NO: 28 - protein, 21 aa, Homo sapiens
MALPVTALLLPLALLLHAARP

SEQ ID NO: 29 - protein, 20 aa, Homo sapiens
METDTLLLWVLLLWVPGSTG

SEQ ID NO: 30 - protein, 22 aa, Homo sapiens
MLLLVTSLLLCELPHPAFLLIP

SEQ ID NO: 31 - protein, 21 aa, Homo sapiens
MEFGLSWLFLVAILKGVQCSR

SEQ ID NO: 32 - protein, 15 aa, synthetic construct
GGGGSGGGGSGGGGS

SEQ ID NO: 33 - protein, 18 aa, synthetic construct
GSTSGSGKPGSGEGSTKG

SEQ ID NO: 34 - protein, 45 aa, Homo sapiens
TTTPAPRPPTPAPTIASQPLSLRPEACRPAAGGAVHTRGLDFACD

SEQ ID NO: 35 - protein, 39 aa, Homo sapiens
IEVMYPPPYLDNEKSNGTIIHVKGKHLCPSPLFPGPSKP

SEQ ID NO: 36 - protein, 42 aa, Homo sapiens
AAAIEVMYPPPYLDNEKSNGTIIHVKGKHLCPSPLFPGPSKP

SEQ ID NO: 37 - protein, 12 aa, Homo sapiens
ESKYGPPCPPCP

SEQ ID NO: 38 - protein, 12 aa, Homo sapiens
ESKYGPPCPSCP

SEQ ID NO: 39 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 24 aa, Homo sapiens
IYIWAPLAGTCGVLLLSLVITLYC

SEQ ID NO: 41 - protein, 27 aa, Homo sapiens
FWVLVVVGGVLACYSLLVTVAFIIFWV

SEQ ID NO: 42 - protein, 28 aa, Homo sapiens
MFWVLVVVGGVLACYSLLVTVAFIIFWV

SEQ ID NO: 43 - protein, 42 aa, Homo sapiens
KRGRKKLLYIFKQPFMRPVQTTQEEDGCSCRFPEEEEGGCEL

SEQ ID NO: 44 - protein, 41 aa, Homo sapiens
RSKRSRLLHSDYMNMTPRRPGPTRKHYQPYAPPRDFAAYRS

SEQ ID NO: 45 - protein, 41 aa, Homo sapiens
RSKRSRGGHSDYMNMTPRRPGPTRKHYQPYAPPRDFAAYRS

SEQ ID NO: 46 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 25 aa, synthetic construct
TTTPAPRPPTPAPTIASQPLSLRPE

SEQ ID NO: 49 - protein, 44 aa, synthetic construct
ACRPAAGGAVHTRGLDFACDIYIWAPLAGTCGVLLLSLVITLYC

SEQ ID NO: 50 - protein, 5 aa, synthetic construct
SYWIE

SEQ ID NO: 51 - protein, 5 aa, synthetic construct
DYVIS

SEQ ID NO: 52 - protein, 5 aa, synthetic construct
THWMH

SEQ ID NO: 53 - protein, 5 aa, synthetic construct
GYWMY

SEQ ID NO: 54 - protein, 17 aa, synthetic construct
EILPGSGSTSYNEKFKG

SEQ ID NO: 55 - protein, 17 aa, synthetic construct
EIYPGSGSSYYNEKFKG

SEQ ID NO: 56 - protein, 17 aa, synthetic construct
MINPSDGVTYYAQTFQG

SEQ ID NO: 57 - protein, 17 aa, synthetic construct
AISPGGGSTYYPDSVKG

SEQ ID NO: 58 - protein, 11 aa, synthetic construct
RGYGYDEGFDY

SEQ ID NO: 59 - protein, 8 aa, synthetic construct
PGDLGFAY

SEQ ID NO: 60 - protein, 9 aa, synthetic construct
EYYGEGFDY

SEQ ID NO: 61 - protein, 12 aa, synthetic construct
SLTATHTYEYDY

SEQ ID NO: 62 - protein, 10 aa, synthetic construct
ASQDINSYLS

SEQ ID NO: 63 - protein, 14 aa, synthetic construct
ASQSVDYDGDSYMN

SEQ ID NO: 64 - protein, 11 aa, synthetic construct
RASQGISNYLA

SEQ ID NO: 65 - protein, 7 aa, synthetic construct
RANRLVD

SEQ ID NO: 66 - protein, 7 aa, synthetic construct
AASNLES

SEQ ID NO: 67 - protein, 7 aa, synthetic construct
SASNLQS

SEQ ID NO: 68 - protein, 9 aa, synthetic construct
LQYDEFPPT

SEQ ID NO: 69 - protein, 9 aa, synthetic construct
QQSNKDPFT

SEQ ID NO: 70 - protein, 9 aa, synthetic construct
QQSYSTPLT

SEQ ID NO: 71 - protein, 120 aa, synthetic construct
QVQLQQSGAELMKPGASVKISCKATGYTFSSYWIEWVKQRPGHGLEWIGEILPGSGSTSYNEKFKGKATFTADTSSNTAYMQLSSLTSEDSAVYYCARRGYGYDEGFDYWGQGTTLTVSS

SEQ ID NO: 72 - protein, 117 aa, synthetic construct
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSS

SEQ ID NO: 73 - protein, 118 aa, synthetic construct
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSS

SEQ ID NO: 74 - protein, 121 aa, synthetic construct
EVQLVESGGGLVQSGGSLRLSCAASGFTFSGYWMYWVRQAPGKGLEWVSAISPGGGSTYYPDSVKGRFTISRDNAKNTLYLQMNSLEPEDTALYYCASSLTATHTYEYDYWGQGTQVTVSS

SEQ ID NO: 75 - protein, 108 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 76 - protein, 112 aa, synthetic construct
DIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 77 - protein, 108 aa, synthetic construct
DIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 78 - protein, 5 aa, synthetic construct
SYAIS

SEQ ID NO: 79 - protein, 5 aa, synthetic construct
DYYIQ

SEQ ID NO: 80 - protein, 5 aa, synthetic construct
SYYMH

SEQ ID NO: 81 - protein, 5 aa, synthetic construct
NHYMH

SEQ ID NO: 82 - protein, 17 aa, synthetic construct
IIDPSDGNTNYAQNFQG

SEQ ID NO: 83 - protein, 17 aa, synthetic construct
WINPNSGGTSYAQKFQG

SEQ ID NO: 84 - protein, 17 aa, synthetic construct
GFDPEDGETIYAQKFQG

SEQ ID NO: 85 - protein, 17 aa, synthetic construct
WMNPNSGNTGYAQKFQG

SEQ ID NO: 86 - protein, 13 aa, synthetic construct
ERAAAGYYYYMDV

SEQ ID NO: 87 - protein, 10 aa, synthetic construct
EGDYYYGMDA

SEQ ID NO: 88 - protein, 8 aa, synthetic construct
DQGWGMDV

SEQ ID NO: 89 - protein, 9 aa, synthetic construct
SESGSDLDY

SEQ ID NO: 90 - protein, 11 aa, synthetic construct
RASQSISSYLN

SEQ ID NO: 91 - protein, 16 aa, synthetic construct
RSSQSLLHSNGYNYLD

SEQ ID NO: 92 - protein, 11 aa, synthetic construct
RASQSISSYLN

SEQ ID NO: 93 - protein, 11 aa, synthetic construct
RASQTIGNYVN

SEQ ID NO: 94 - protein, 7 aa, synthetic construct
AASSLQS

SEQ ID NO: 95 - protein, 7 aa, synthetic construct
LGSNRAS

SEQ ID NO: 96 - protein, 7 aa, synthetic construct
AASSLQS

SEQ ID NO: 97 - protein, 7 aa, synthetic construct
GASNLHT

SEQ ID NO: 98 - protein, 9 aa, synthetic construct
QQSYSTPLT

SEQ ID NO: 99 - protein, 9 aa, synthetic construct
MQGLQTPHT

SEQ ID NO: 100 - protein, 9 aa, synthetic construct
QQTYSTPYT

SEQ ID NO: 101 - protein, 9 aa, synthetic construct
QQTYSAPLT

SEQ ID NO: 102 - protein, 122 aa, synthetic construct
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSS

SEQ ID NO: 103 - protein, 119 aa, synthetic construct
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSS

SEQ ID NO: 104 - protein, 117 aa, synthetic construct
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSS

SEQ ID NO: 105 - protein, 118 aa, synthetic construct
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSS

SEQ ID NO: 106 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 107 - protein, 113 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 108 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 109 - protein, 108 aa, synthetic construct
DIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 110 - protein, 546 aa, Nipah virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 111 - protein, 520 aa, Nipah virus
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 112 - protein, 83 aa, Nipah virus
ILHYEKLSKIGLVKGVTRKYKIKSNPLTKDIVIKMIPNVSNMSQCTGSVMENYKTRLNGILTPIKGALEIYKNNTHDLVGDVR

SEQ ID NO: 113 - protein, 437 aa, Nipah virus
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 114 - protein, 500 aa, Nipah virus
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 115 - protein, 417 aa, Nipah virus
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 116 - protein, 500 aa, Nipah virus
ILHYEKLSKIGLVKGVTRKYKIKSNPLTKDIVIKMIPNVSNMSQCTGSVMENYKTRLNGILTPIKGALEIYKNQTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 117 - protein, 524 aa, Nipah virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 118 - protein, 498 aa, Nipah virus
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 119 - protein, 415 aa, Nipah virus
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 120 - protein, 602 aa, Nipah virus
MGPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 121 - protein, 597 aa, Nipah virus
MGKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 122 - protein, 592 aa, Nipah virus
MGNTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 123 - protein, 587 aa, Nipah virus
MGKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 124 - protein, 582 aa, Nipah virus
MGSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 125 - protein, 577 aa, Nipah virus
MGSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 126 - protein, 573 aa, Nipah virus
MGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 127 - protein, 569 aa, Nipah virus
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 128 - protein, 546 aa, Henipavirus hendraense
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 129 - protein, 604 aa, Henipavirus hendraense
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 130 - protein, 526 aa, Nipah virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 131 - protein, 526 aa, Nipah virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNQTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 132 - protein, 569 aa, Nipah virus
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 133 - protein, 498 aa, Nipah virus; identical to SEQ ID NO: 118

SEQ ID NO: 134 - protein, 557 aa, unidentified
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 135 - protein, 545 aa, unidentified
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 136 - protein, 662 aa, unidentified
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 137 - protein, 36 aa, synthetic construct
  VARIANT 1..36: note = This sequence may encompass 1-6 GGGGGS repeating units
GGGGGSGGGGGSGGGGGSGGGGGSGGGGGSGGGGGS

SEQ ID NO: 138 - protein, 602 aa, Nipah virus
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 139 - protein, 622 aa, unidentified
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 140 - protein, 632 aa, unidentified
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 141 - protein, 625 aa, unidentified
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 142 - protein, 552 aa, Nipah virus
FNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 143 - protein, 555 aa, Henipavirus hendraense
FNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 144 - protein, 26 aa, synthetic construct
MVVILDKRCYCNLLILILMISECSVG

SEQ ID NO: 145 - protein, 6 aa, synthetic construct
GGGGGS

SEQ ID NO: 146 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 147 - protein, 5 aa, synthetic construct
GGGGS

SEQ ID NO: 148 - protein, 600 aa, Nipah virus
PAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 149 - protein, 595 aa, Nipah virus
KVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 150 - protein, 590 aa, Nipah virus
NTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 151 - protein, 585 aa, Nipah virus
KGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 152 - protein, 580 aa, Nipah virus
SKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 153 - protein, 575 aa, Nipah virus
SYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 154 - protein, 571 aa, Nipah virus
TMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 155 - protein, 568 aa, Nipah virus
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 156 - protein, 603 aa, Henipavirus hendraense
MADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 157 - protein, 568 aa, Nipah virus
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 158 - protein, 621 aa, unidentified
LSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 159 - protein, 631 aa, unidentified
PQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 160 - protein, 624 aa, unidentified
ATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 161 - protein, 530 aa, unidentified
DFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 162 - protein, 640 aa, unidentified
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 163 - protein, 520 aa, Henipavirus hendraense
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 164 - protein, 519 aa, unidentified
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 165 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 166 - protein, 15 aa, synthetic construct
TTAASGSSGGSSSGA

SEQ ID NO: 167 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 168 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 169 - DNA, 972 bp, Homo sapiens
  source 1..972: mol_type = genomic DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 170 - DNA, 918 bp, Homo sapiens
  source 1..918: mol_type = genomic DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 171 - DNA, 969 bp, synthetic construct
  source 1..969: mol_type = other DNA
atgtggcccctggtcgccgccctgttgctgggctcggcatgctgcggatcagctcagctactgtttaataaaacaaaatctgtagaattcacgttttgtaacgacactgtcgtgatcccatgctttgttactaatatggaggcacaaaacaccactgaagtgtacgtgaagtggaaattcaaaggcagagacatttacacctttgacggcgccctcaacaagtccaccgtgcccactgactttagtagcgcaaaaattgaggtcagccaattactaaaaggagatgcctctttgaagatggacaagagcgatgctgtcagccacacagggaactacacttgtgaagtaacagagttaacccgcgaaggtgaaacgatcatcgagctgaagtatcgagtggtgtcctggttttctccgaacgagaatatccttatcgtaattttcccaattttcgctatcctcctgttctggggccagtttggtatcaagacactcaaatatcggtccggtgggatggatgagaagacaattgccctgcttgttgctggactcgtgatcaccgtcatcgtgattgttggggccatccttttcgtcccaggggagtacagcctgaagaatgctacgggcctgggattaattgtgacctctacagggatactcatcctgcttcactactatgtgttcagtaccgcgattggactgacctccttcgtcattgccatattggtgattcaggtgatagcctacatcctcgccgtggttggcctgagtctctgtatcgcggcgtgcatacccatgcatggccctcttctgatttcagggttgagtatcctcgcactagcacagttgctgggactggtttatatgaaatttgtggcctccaaccagaagactatacagcctcctaggaaggctgtagaggagcccctgaatgcattcaaggaatcaaaaggcatgatgaatgatgaa

SEQ ID NO: 172 - protein, 21 aa, synthetic construct
  VARIANT 1..3: note = May be absent
GSGEGRGSLLTCGDVEENPGP

SEQ ID NO: 173 - protein, 22 aa, synthetic construct
  VARIANT 1..3: note = May be absent
GSGATNFSLLKQAGDVEENPGP

SEQ ID NO: 174 - protein, 23 aa, synthetic construct
  VARIANT 1..3: note = May be absent
GSGQCTNYALLKLAGDVESNPGP

SEQ ID NO: 175 - protein, 25 aa, synthetic construct
  VARIANT 1..3: note = May be absent
GSGVKQTLNFDLLKLAGDVESNPGP

SEQ ID NO: 176 - protein, 7 aa, Homo sapiens
  VARIANT 5..7: note = May be absent
RRRRGSG

SEQ ID NO: 177 - protein, 7 aa, Homo sapiens
  VARIANT 5..7: note = May be absent
RKRRGSG

SEQ ID NO: 178 - protein, 15 aa, Homo sapiens
  VARIANT 5..7: note = May be absent
  VARIANT 13..15: note = May be absent
RKRRGSGTPDPWGSG

SEQ ID NO: 179 - skipped

SEQ ID NO: 180 - RNA, 12 nt, synthetic construct
  source 1..12: mol_type = other RNA
gttttagagcta

SEQ ID NO: 181 - skipped

SEQ ID NO: 182 - RNA, 66 nt, synthetic construct
  source 1..66: mol_type = other RNA
tagcaagttaaaataaggctagtccgttatcaacttgaaaaagtggcaccgagtcggtgctttttt

SEQ ID NO: 183 - skipped

SEQ ID NO: 184 - RNA, 17 nt, synthetic construct
  source 1..17: mol_type = other RNA
gtttsagagctatgctg

SEQ ID NO: 185 - skipped

SEQ ID NO: 186 - RNA, 71 nt, synthetic construct
  source 1..71: mol_type = other RNA
cagcatagcaagttsaaataaggctagtccgttatcaacttgaaaaagtggcaccgagtcggtgctttttt

SEQ ID NO: 187 - skipped

SEQ ID NO: 188 - RNA, 14 nt, synthetic construct
  source 1..14: mol_type = other RNA
gttttagtactctg

SEQ ID NO: 189 - skipped

SEQ ID NO: 190 - RNA, 64 nt, synthetic construct
  source 1..64: mol_type = other RNA
cagaatctactaaaacaaggcaaaatgccgtgtttatctcgtcaacttgttggcgagatttttt

SEQ ID NO: 191 - RNA, 88 nt, synthetic construct
  source 1..88: mol_type = other RNA
gtcgtctataggacggcgaggacaacgggaagtgccaatgtgctctttccaagagcaaacaccccgttggcttcaagatgaccgctcg

SEQ ID NO: 192 - skipped

SEQ ID NO: 193 - RNA, 23 nt, synthetic construct
  source 1..23: mol_type = other RNA
cgagcggtctgagaagtggcact

SEQ ID NO: 194 - skipped

SEQ ID NO: 195 - protein, 7 aa, synthetic construct
GFTVSSN

SEQ ID NO: 196 - protein, 7 aa, synthetic construct
GFTFSDY

SEQ ID NO: 197 - protein, 7 aa, synthetic construct
GYSFTSY

SEQ ID NO: 198 - protein, 5 aa, synthetic construct
YSGGS

SEQ ID NO: 199 - protein, 6 aa, synthetic construct
SSSGST

SEQ ID NO: 200 - protein, 6 aa, synthetic construct
YPGDSD

SEQ ID NO: 201 - protein, 14 aa, synthetic construct
ARDGGLDTLYGMDV

SEQ ID NO: 202 - protein, 11 aa, synthetic construct
ARVEGWSLLDY

SEQ ID NO: 203 - protein, 15 aa, synthetic construct
ARLPFVGATTGGFDY

SEQ ID NO: 204 - protein, 14 aa, synthetic construct
TGSSSNIGAGYDVH

SEQ ID NO: 205 - protein, 13 aa, synthetic construct
SGSSSNIGNNYVS

SEQ ID NO: 206 - protein, 14 aa, synthetic construct
TGTSSDVGGYNYVS

SEQ ID NO: 207 - protein, 12 aa, synthetic construct
KLLIYGNSNRPS

SEQ ID NO: 208 - protein, 12 aa, synthetic construct
KLLIYDNNKRPS

SEQ ID NO: 209 - protein, 12 aa, synthetic construct
KLVIYEVSNRPS

SEQ ID NO: 210 - protein, 12 aa, synthetic construct
QSYDSSLSGSYV

SEQ ID NO: 211 - protein, 11 aa, synthetic construct
GTWDSSLGAVV

SEQ ID NO: 212 - protein, 10 aa, synthetic construct
SSYTSSSTLV

SEQ ID NO: 213 - protein, 8 aa, synthetic construct
GFTVSSNY

SEQ ID NO: 214 - protein, 8 aa, synthetic construct
GFTFSDYY

SEQ ID NO: 215 - protein, 8 aa, synthetic construct
GYSFTSYW

SEQ ID NO: 216 - protein, 7 aa, synthetic construct
IYSGGST

SEQ ID NO: 217 - protein, 8 aa, synthetic construct
ISSSGSTT

SEQ ID NO: 218 - protein, 8 aa, synthetic construct
IYPGDSDT

SEQ ID NO: 219 - protein, 9 aa, synthetic construct
SSNIGAGYD

SEQ ID NO: 220 - protein, 8 aa, synthetic construct
SSNIGNNY

SEQ ID NO: 221 - protein, 9 aa, synthetic construct
SSDVGGYNY

SEQ ID NO: 222-226 - skipped (5 entries)

SEQ ID NO: 227 - protein, 4 aa, synthetic construct
GGGS

SEQ ID NO: 228 - protein, 247 aa, synthetic construct
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKVTVLGGGGSGGGGSGGGGSEVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDGGLDTLYGMDVWGQGTTVTVSS

SEQ ID NO: 229 - protein, 243 aa, synthetic construct
QSVLTQPPSVSAAPGQKVTMSCSGSSSNIGNNYVSWYQQLPGTAPKLLIYDNNKRPSGIPDRFSGSKSGTSATLGITGLQTGDEADYYCGTWDSSLGAVVFGGGTKVTVLGGGGSGGGGSGGGGSEVHLVESGGGLVQPGGSLRLSCAASGFTFSDYYMSWIRQAPGKGLEWVSYISSSGSTTYYADSVKGRFTISRDNAKNSLYLQMNSLRAEDTAVYYCARVEGWSLLDYWGQGTLVTVSS

SEQ ID NO: 230 - protein, 247 aa, synthetic construct
QSVVTQPASVSGSPGQSITISCTGTSSDVGGYNYVSWYQQHPGKAPKLVIYEVSNRPSGVSNRFSGSKSGNTASLTISGLQAEDEADYYCSSYTSSSTLVFGGGTKLTVLGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLPFVGATTGGFDYWGQGTLVTVSS

SEQ ID NO: 231 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 232 - protein, 470 aa, synthetic construct
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDGGLDTLYGMDVWGQGTTVTVSSGGGGSGGGGSGGGGSQSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 233 - DNA, 1410 bp, synthetic construct
  source 1..1410: mol_type = other DNA
gaagtgcagctggtggagtctggaggaggcttgatccagcctggggggtccctgagactctcctgtgcagcctctgggttcaccgtcagtagcaactacatgagctgggttcgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagagatggaggtctcgatactctctacggtatggacgtctggggccaagggaccacggtcaccgtctcctcaggtggaggcggttccggaggcggtggcagcggcggtggcggttcgcagtctgtcgtgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttcgtatgtcttcggaactgggaccaaggtgaccgtccttaccactacaccagcccctaggcctccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 234 - protein, 466 aa, synthetic construct
EVHLVESGGGLVQPGGSLRLSCAASGFTFSDYYMSWIRQAPGKGLEWVSYISSSGSTTYYADSVKGRFTISRDNAKNSLYLQMNSLRAEDTAVYYCARVEGWSLLDYWGQGTLVTVSSGGGGSGGGGSGGGGSQSVLTQPPSVSAAPGQKVTMSCSGSSSNIGNNYVSWYQQLPGTAPKLLIYDNNKRPSGIPDRFSGSKSGTSATLGITGLQTGDEADYYCGTWDSSLGAVVFGGGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 235 - DNA, 1398 bp, synthetic construct
  source 1..1398: mol_type = other DNA
gaggtgcatctggtggagtctgggggaggcttggtacagccgggggggtccctgagactctcctgtgcagcctctggattcaccttcagtgactactacatgagctggatccgccaggctccagggaaggggctggagtgggtttcatacattagtagtagtggtagtaccacatactacgcagactccgtgaagggccggttcaccatctccagagacaacgccaagaactcactgtatctgcaaatgaacagcctgagagccgaggacacggccgtgtattactgtgcgagagtagaaggctggagtcttttggactactggggccagggaaccctggtcactgtctcctcaggtggaggcggttccggaggcggtggcagcggcggtggcggttcgcagtctgtgttgacgcagccgccctcagtgtctgcggccccaggacagaaggtcaccatgtcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattattgcggaacatgggatagtagcctgggtgctgtggtattcggcggggggaccaaggtgaccgtcctcaccactacaccagcccctaggcctccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 236 - protein, 470 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLPFVGATTGGFDYWGQGTLVTVSSGGGGSGGGGSGGGGSQSVVTQPASVSGSPGQSITISCTGTSSDVGGYNYVSWYQQHPGKAPKLVIYEVSNRPSGVSNRFSGSKSGNTASLTISGLQAEDEADYYCSSYTSSSTLVFGGGTKLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 237 - DNA, 1410 bp, synthetic construct
  source 1..1410: mol_type = other DNA
gaagtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacttccttttgtgggagctaccacggggggctttgactactggggccagggaaccctggtcaccgtctcctcaggtggaggcggttccggaggcggtggcagcggcggtggcggttcgcagtctgtcgtgacgcagcctgcctccgtgtctgggtctcctggacagtcgatcaccatctcctgcactggaaccagcagtgacgttggtggttataactatgtctcctggtaccaacagcacccaggcaaagcccccaaactcgtgatttatgaggtcagtaatcggccctcaggggtttctaatcgcttctctggctccaagtctggcaacacggcctccctgaccatctctgggctccaggctgaggacgaggctgattattactgcagctcatatacaagcagcagcactctggtgttcggcggagggaccaagctgaccgtcctaaccactacaccagcccctaggcctccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 238 - protein, 470 aa, synthetic construct
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKVTVLGGGGSGGGGSGGGGSEVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDGGLDTLYGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 239 - DNA, 1410 bp, synthetic construct
  source 1..1410: mol_type = other DNA
cagtctgtcgtgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttcgtatgtcttcggaactgggaccaaggtgaccgtccttggtggaggcggttccggaggcggtggcagcggcggtggcggttcggaagtgcagctggtggagtctggaggaggcttgatccagcctggggggtccctgagactctcctgtgcagcctctgggttcaccgtcagtagcaactacatgagctgggttcgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagagatggaggtctcgatactctctacggtatggacgtctggggccaagggaccacggtcaccgtctcctcaaccactacaccagcccctaggcctccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 240 - protein, 466 aa, synthetic construct
QSVLTQPPSVSAAPGQKVTMSCSGSSSNIGNNYVSWYQQLPGTAPKLLIYDNNKRPSGIPDRFSGSKSGTSATLGITGLQTGDEADYYCGTWDSSLGAVVFGGGTKVTVLGGGGSGGGGSGGGGSEVHLVESGGGLVQPGGSLRLSCAASGFTFSDYYMSWIRQAPGKGLEWVSYISSSGSTTYYADSVKGRFTISRDNAKNSLYLQMNSLRAEDTAVYYCARVEGWSLLDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 241 - DNA, 1398 bp, synthetic construct
  source 1..1398: mol_type = other DNA
cagtctgtgttgacgcagccgccctcagtgtctgcggccccaggacagaaggtcaccatgtcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattattgcggaacatgggatagtagcctgggtgctgtggtattcggcggggggaccaaggtgaccgtcctcggtggaggcggttccggaggcggtggcagcggcggtggcggttcggaggtgcatctggtggagtctgggggaggcttggtacagccgggggggtccctgagactctcctgtgcagcctctggattcaccttcagtgactactacatgagctggatccgccaggctccagggaaggggctggagtgggtttcatacattagtagtagtggtagtaccacatactacgcagactccgtgaagggccggttcaccatctccagagacaacgccaagaactcactgtatctgcaaatgaacagcctgagagccgaggacacggccgtgtattactgtgcgagagtagaaggctggagtcttttggactactggggccagggaaccctggtcactgtctcctcaaccactacaccagcccctaggcctccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 242 - protein, 470 aa, synthetic construct
QSVVTQPASVSGSPGQSITISCTGTSSDVGGYNYVSWYQQHPGKAPKLVIYEVSNRPSGVSNRFSGSKSGNTASLTISGLQAEDEADYYCSSYTSSSTLVFGGGTKLTVLGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLPFVGATTGGFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 243 - DNA, 1410 bp, synthetic construct
  source 1..1410: mol_type = other DNA
cagtctgtcgtgacgcagcctgcctccgtgtctgggtctcctggacagtcgatcaccatctcctgcactggaaccagtagtgacgttggtggttataactatgtctcctggtaccaacagcacccaggcaaagcccccaaactcgtgatttatgaggtcagtaatcggccctcaggggtttctaatcgcttctctggctccaagtctggcaacacggcctccctgaccatctctgggctccaggctgaggacgaggctgattattactgcagctcatatacaagcagcagcactctggtgttcggcggagggaccaagctgaccgtcctaggtggaggcggttccggaggcggtggcagcggcggtggcggttcggaagtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacttccttttgtgggagctaccacggggggctttgactactggggccagggaaccctggtcactgtctcctcaaccactacaccagcccctaggcctccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc